(19) United States
(12) Patent Application Publication
(10) Pub. No.: US 2017/0012216 A1
(43) Pub. Date: Jan. 12, 2017

(54) CONDENSED CYCLIC COMPOUND AND ORGANIC LIGHT-EMITTING DEVICE INCLUDING THE SAME

(71) Applicants: SAMSUNG SDI CO., LTD., Gyeonggi-do (KR); SAMSUNG ELECTRONICS CO., LTD., Gyeonggi-do (KR)

(72) Inventors: Byung-Ku KIM, Suwon-si, Gyeonggi-do (KR); Jin-Hyun LUI, Suwon-si, Gyeonggi-do (KR); Byoung-Kwan LEE, Suwon-si, Gyeonggi-do (KR); O Hyun KWON, Yongin-si, Gyeonggi-do (KR); Young-Kwon KIM, Suwon-si, Gyeonggi-do (KR); Chang-Woo KIM, Suwon-si, Gyeonggi-do (KR); Hyung-Sun KIM, Suwon-si, Gyeonggi-do (KR); Joo-Hee SEO, Suwon-si, Gyeonggi-do (KR); Chang-Ju SHIN, Suwon-si, Gyeonggi-do (KR); Eun-Sun YU, Suwon-si, Gyeonggi-do (KR); Seung-Jae LEE, Suwon-si, Gyeonggi-do (KR); Byoung Ki CHOI, Hwaseong-si, Gyeonggi-do (KR); Kyu Young HWANG, Ansan-si, Gyeonggi-do (KR)

(21) Appl. No.: 15/107,349

(22) PCT Filed: Jan. 6, 2015
(86) PCT No.: PCT/KR2015/000112

§ 371 Date: Jun. 22, 2016

(30) Foreign Application Priority Data
Jan. 10, 2014 (KR) 10-2014-0003604
Jan. 10, 2014 (KR) 10-2014-0003605

(51) Int. Cl. H01L 51/00; C07D 495/04; C07D 491/048; C07D 409/14; C07D 487/04; C07D 209/86; C07D 401/14; C07D 405/14; C09K 11/02; C07F 7/08

(52) U.S. Cl. CPC H01L 51/0071; C09K 11/025; C07D 495/04; H01L 51/0054; H01L 51/0052; C07D 491/048; C07F 7/0816; H01L 51/0094; C07D 487/04; H01L 51/0072; C07D 209/86; C07D 401/14; H01L 51/0067; C07D 405/14; H01L 51/0073; C07D 409/14; H01L 51/0074; H01L 51/508

(57) ABSTRACT

A condensed cyclic compound and an organic light-emitting device including the condensed cyclic compound are provided.

### TECHNICAL FIELD

One or more embodiments of the present disclosure relate to a condensed cyclic compound, and an organic light-emitting device including the same.

### BACKGROUND ART

Organic light-emitting devices (OLEDs), which are self-emitting devices, have advantages such as wide viewing angles, excellent contrast, quick response, high brightness, excellent driving voltage characteristics, and can provide multicolored images.

An organic light-emitting device may include an anode, a cathode, and an organic layer including an emission layer and disposed between the anode and the cathode. The organic light-emitting device may include a hole transport region between the anode and the emission layer, and an electron transport region between the emission layer and the cathode. Holes injected from the anode move to the emission layer via the hole transport region, while electrons injected from the cathode move to the emission layer via the electron transport region. Carriers such as the holes and electrons recombine in the emission layer to generate excitons. When the excitons drop from an excited state to a ground state, light is emitted.

### DISCLOSURE

### Technical Problem

One or more embodiments of the present disclosure include a novel condensed cyclic compound, and an organic light-emitting device including the same.

The light-emitting device includes compounds different from each other, for example as hosts, and thus has a lower driving voltage, high efficiency, high luminance and long life-span characteristics.

The compound is used in an electron transport auxiliary layer to provide a light-emitting device having a lower driving voltage, high efficiency, high luminance and long life-span characteristics.

### Technical Solution

According to one or more embodiments of the present disclosure, there is provided a condensed cyclic compound represented by Formula 1:

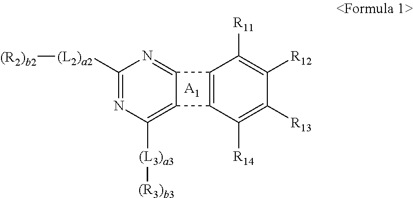

wherein, in Formula 1, ring A₁ is represented by Formula 1A,

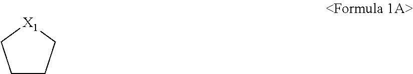

where X₁ is N-[(L₁)ₐ₁-(R₁)_{b1}], S, O, or Si(R₄)(R₅);

L₁ to L₃ are each independently selected from a substituted or unsubstituted C₆-C₆₀ arylene group a1 to a3 are each independently an integer selected from 0 to 5,

R₁ to R₅ are each independently selected from a hydrogen, a deuterium, a fluoro group (—F), a chloro group (—Cl), a bromo group (—Br), an iodo group (—I), a hydroxyl group, a substituted or unsubstituted C₁-C₆₀ alkyl group, a substituted or unsubstituted C₁-C₆₀ alkoxy group, a substituted or unsubstituted C₃-C₁₀ cycloalkyl group, a substituted or unsubstituted C₆-C₆₀ aryl group, a substituted or unsubstituted C₆-C₆₀ aryloxy group, a substituted or unsubstituted C₆-C₆₀ arylthio group, and a substituted or unsubstituted monovalent non-aromatic condensed polycyclic group, wherein at least one of R₂ and R₃ is selected from a substituted or unsubstituted C₆-C₆₀ aryl group, and a substituted or unsubstituted monovalent non-aromatic condensed polycyclic group,

R₁₁ to R₁₄ are each independently selected from a hydrogen, a deuterium, —F, —Cl, —Br, —I, a hydroxyl group, a substituted or unsubstituted C₁-C₆₀ alkyl group, a substituted or unsubstituted C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, and

b1 to b3 are each independently an integer selected from 1 to 3,

when R₂ is a substituted or unsubstituted phenyl group, R₃ is selected from a hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted fluoranthenyl group, a substituted or unsubstituted triphenylenyl group, a substituted or unsubstituted pyrenyl group, a substituted or unsubstituted phenanthrenyl group, a substituted or unsubstituted fluorenyl group, and a substituted or unsubstituted chrysenyl group;

at least one of substituents of the substituted C₁-C₆₀ alkyl group, the substituted C₁-C₆₀ alkoxy group, the substituted C₃-C₁₀ cycloalkyl group, the substituted C₆-C₆₀ aryl group, the substituted C₆-C₆₀ aryloxy group, the substituted C₆-C₆₀ arylthio group, and the substituted monovalent non-aromatic condensed polycyclic group is selected from

a deuterium, —F, —Cl, —Br, —I, a hydroxyl group, a C₁-C₆₀ alkyl group, and a C₁-C₆₀ alkoxy group,

a C₁-C₆₀ alkyl group, and a C₁-C₆₀ alkoxy group, each substituted with at least one of a deuterium, —F, —Cl, —Br, —I, a hydroxyl group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group,

a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group,

a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, each substituted with at least one of a deuterium, —F, —Cl, —Br, —I, a hydroxyl group, a C₁-C₆₀ alkyl group, a C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, and

a substituent of R₂ and R₃ is selected from a deuterium, —F, —Cl, —Br, —I, a hydroxyl group, a C₁-C₆₀ alkyl group, a C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group.

According to one or more embodiments of the present disclosure, an organic light-emitting device includes a first electrode, a second electrode, and an organic layer disposed between the first electrode and the second electrode and the organic layer includes the condensed cyclic compounds of Formula 1 defined above.

The condensed cyclic compounds of Formula 1 may be included in the emission layer or electron transport auxiliary layer of the organic layer, and the emission layer may further include a dopant. The condensed cyclic compounds of Formula 1 in the emission layer may serve as a host.

According to one or more embodiments of the present disclosure, an organic light-emitting device includes an organic layer including i) the condensed cyclic compound represented by the following Formula 1 and at least one of ii) a first compound represented by Formula 41 and a second compound represented by the following Formula 61.

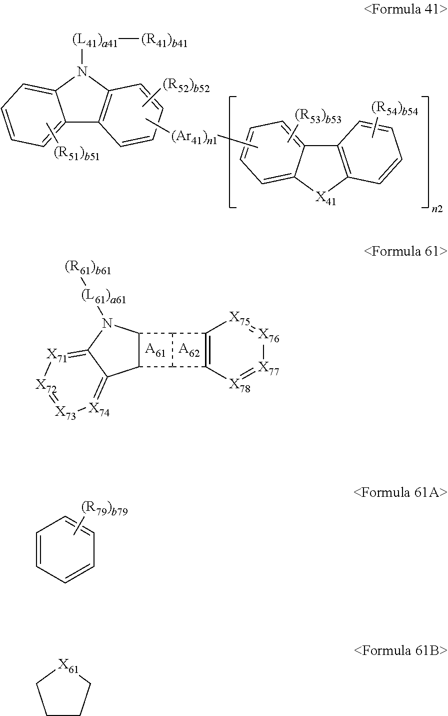

In Formula 41, X₄₁ is N-[(L₄₂)ₐ₄₂-(R₄₂)_{b42}], S, O, S(═O), S(═O)₂, C(═O), C(R₄₃)(R₄₄), Si(R₄₃)(R₄₄), P(R₄₃), P(═O)(R₄₃) or C═N(R₄₃);

in Formula 61, the ring A₆₁ is represented by Formula 61A;

in Formula 61, the ring A₆₂ is represented by Formula 61B;

X₆₁ is N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)(R₆₃) or C═N(R₆₃);

X₇₁ is C(R₇₁) or N, X₇₂ is C(R₇₂) or N, X₇₃ is C(R₇₃) or N, X₇₄ is C(R₇₄) or N, X₇₅ is C(R₇₅) or N, X₇₆ is C(R₇₆) or N, X₇₇ is C(R₇₇) or N, and X₇₈ is C(R₇₈) or N;

Ar₄₁, L₄₁, L₄₂, L₆₁ and L₆₂ are each independently a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ hetero cycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group or a substituted or unsubstituted divalent non-aromatic heterocondensed polycyclic group;

n1 and n2 are each independently an integer selected from 0 to 3;

a41, a42, a61 and a62 are each independently an integer selected from 0 to 5;

R₄₁ to R₄₄, R₅₁ to R₅₄, R₆₁ to R₆₄ and R₇₁ to R₇₉ are each independently hydrogen, deuterium, —F (a fluoro group), —Cl (a chloro group), —Br (a bromo group), —I (an iodo group), a hydroxyl group, a cyano group, an amino group, an amidino group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅) or —B(Q₆)(Q₇);

b41, b42, b51 to b54, b61, b62 and b79 are each independently an integer selected from 1 to 3.

According to another aspect, an organic light-emitting device that includes the condensed cyclic compound in an electron transport auxiliary layer of an organic layer, and further includes a hole transport auxiliary layer including a compound represented by the following Formula 2.

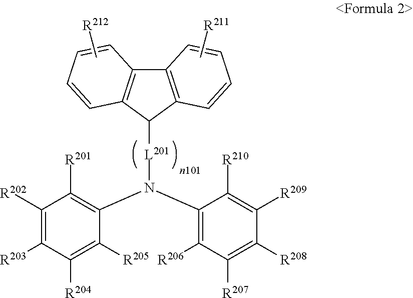

In Formula 2, L²⁰¹ is a substituted or unsubstituted C6 to C30 arylene group, or a substituted or unsubstituted C2 to C30 heteroarylene group, n101 is an integer selected from 1 to 5, R²⁰¹ to R²¹² are each independently hydrogen, a deuterium, a substituted or unsubstituted C1 to C20 alkyl group, a substituted or unsubstituted C6 to C50 aryl group, a substituted or unsubstituted C2 to C50 heteroaryl group or a combination thereof, and R²⁰¹ to R²¹² are each independently present or are fused to each other to form a ring.

### Advantageous Effects

The condensed cyclic compound has a good electrical characteristics and a thermal stability, and thus the organic layer including the condensed cyclic compound of Formula 1 described above, the organic light-emitting device may have a low driving voltage, a high efficiency, and a long lifetime.

### DESCRIPTION OF THE DRAWINGS

FIGS. 1 to 3 are a schematic view of an organic light-emitting device according to an embodiment of the present disclosure.

### DESCRIPTION OF SYMBOLS

10: organic photoelectric device
11: the first electrode
15: organic layer
19: the second electrode
31: hole transport layer (HTL)
32: emission layer
33: hole transport auxiliary layer
34: electron transport layer (ETL)
35: electron transport auxiliary layer
36: electron injection layer (EIL)
37: hole injection layer (HIL)

### MODE FOR INVENTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an embodiment of the present disclosure, there is provided a condensed cyclic compound represented by Formula 1 below:

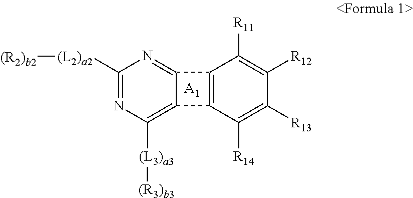

In Formula 1, ring A₁ may be represented by Formula 1A:

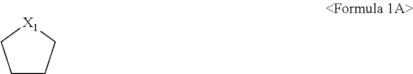

In Formula 1A, X₁ may be N-[(L₁)ₐ₁-(R₁)_{b1}], S, O, or Si(R₄)(R₅).

L₁ to L₃ are each independently selected from a substituted or unsubstituted C₆-C₆₀ arylene group a1 to a3 are each independently an integer selected from 0 to 5,

R₁ to R₅ are each independently selected from a hydrogen, a deuterium, a fluoro group (—F), a chloro group (—Cl), a bromo group (—Br), an iodo group (—I), a hydroxyl group, a substituted or unsubstituted C₁-C₆₀ alkyl group, a substituted or unsubstituted C₁-C₆₀ alkoxy group, a substituted or unsubstituted C₃-C₁₀ cycloalkyl group, a substituted or unsubstituted C₆-C₆₀ aryl group, a substituted or unsubstituted C₆-C₆₀ aryloxy group, a substituted or unsubstituted C₆-C₆₀ arylthio group, and a substituted or unsubstituted monovalent non-aromatic condensed polycyclic group, wherein at least one of R₂ and R₃ is selected from a substituted or unsubstituted C₆-C₆₀ aryl group, and a substituted or unsubstituted monovalent non-aromatic condensed polycyclic group,

R₁₁ to R₁₄ are each independently selected from a hydrogen, a deuterium, —F, —Cl, —Br, —I, a hydroxyl group, a substituted or unsubstituted C₁-C₆₀ alkyl group, a substituted or unsubstituted C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, and

b1 to b3 are each independently an integer selected from 1 to 3,

when R₂ is a substituted or unsubstituted phenyl group, R₃ is selected from a hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted fluoranthenyl group, a substituted or unsubstituted triphenylenyl group, a substituted or unsubstituted pyrenyl group, a substituted or unsubstituted phenanthrenyl group, a substituted or unsubstituted fluorenyl group, and a substituted or unsubstituted chrysenyl group;

The definitions of L₁, a1, R₁, b1, R₄ and R₅ may be the same as those of Formula 1 defined below.

In some embodiments, X₁ may be S, O, or Si(R₄)(R₅), but is not limited thereto. In some other embodiments, X₁ may be S or O, but is not limited thereto.

The ring A₁ may be fused to adjacent two 6-membered rings with shared carbon atoms. Accordingly, the condensed cyclic compound of Formula 1 above may be represented by one of Formulae 1-1 and 1-2:

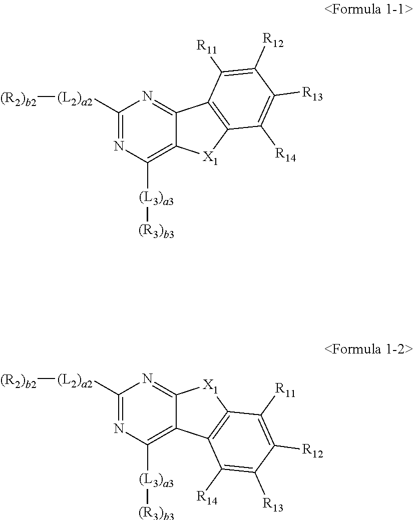

In Formulae 1-1 to 1-2, X₁, L₂, L₃, a2, a3, R₂, R₃, R₁₁ to R₁₄, b2 and b3 may be the same as those of Formula 1 defined below.

In the above Formulae, L₁ to L₃ may be each independently selected from a substituted or unsubstituted C₆-C₆₀ arylene group.

For example, L₁ to L₃ may be each independently selected from

a phenylene group, a biphenylene group, a terphenylene group, a quaterphenylene group, a naphthylene group, a fluorenylene group, a spiro-fluorenylene group, a phenanthrenylene group, an anthracenylene group, a fluoranthrenylene group, a triphenylenylene group, a pyrenylene group, a chrysenylene group, and a naphthacenylene group; and

a phenylene group, a biphenylene group, a terphenylene group, a quaterphenylene group, a naphthylene group, a fluorenylene group, a spiro-fluorenylene group, a phenanthrenylene group, an anthracenylene group, a fluoranthrenylene group, a triphenylenylene group, a pyrenylene group, a chrysenylene group, and a naphthacenylene group, each substituted with at least one of a deuterium atom, —F, —Cl, —Br, —I, a hydroxyl group, C₁-C₂₀ alkyl group, a C₁-C₂₀ alkoxy group, a C₆-C₂₀ aryl group, and a monovalent non-aromatic condensed polycyclic group,

In some other embodiments, in above Formulae, L₁ to L₃ may be each independently represented by one of Formulae 2-1 to 2-15:

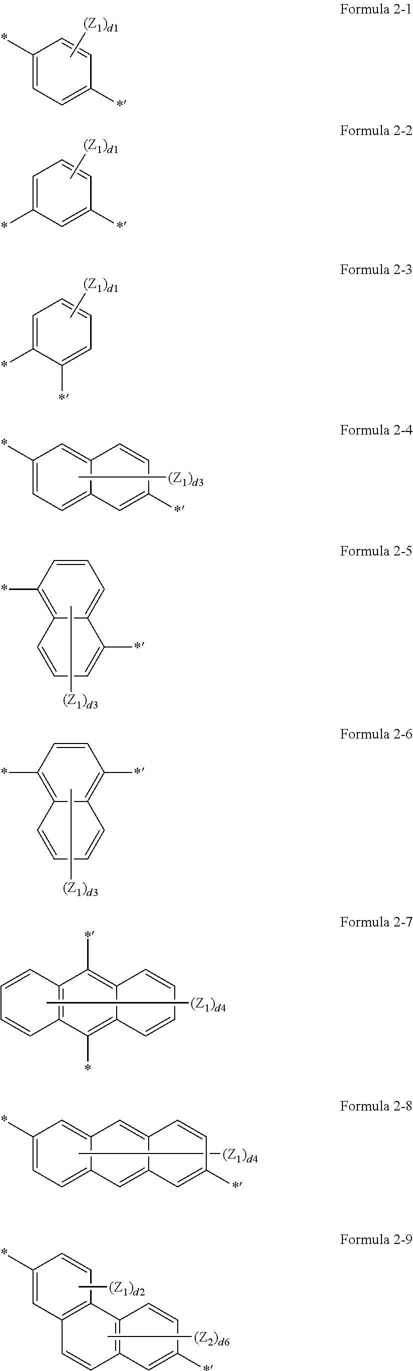

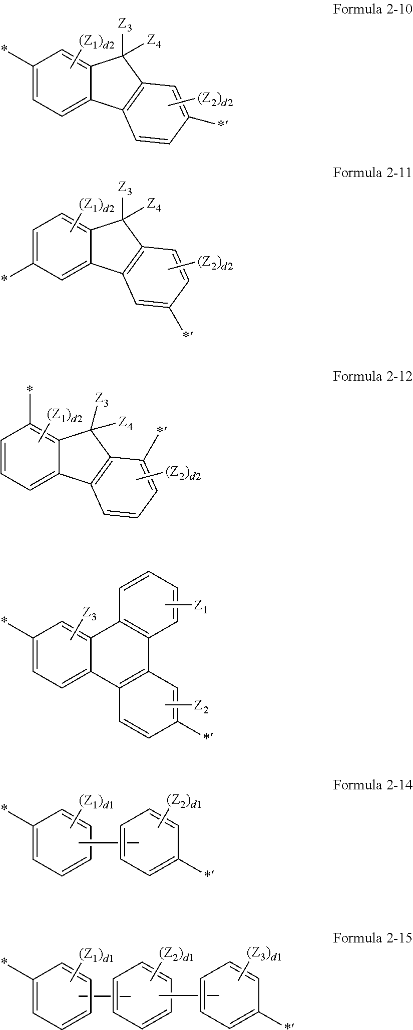

In Formulae 2-1 to 2-15,

Z₁ to Z₄ may be each independently selected from a hydrogen, a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, an anthracenyl group, a fluoranthenyl group, a triphenylenyl group, a pyrenyl group, a phenanthrenyl group, a fluorenyl group, and a chrysenyl group,

d1 may be an integer selected from 1 to 4; d2 may be an integer selected from 1 to 3; d3 may be an integer selected from 1 to 6; d4 may be an integer selected from 1 to 8; d6 may be an integer selected from 1 to 5; and * and *′ may be each independently a binding site with an adjacent atom.

In some other embodiments, in above Formulae, L₁ to L₃ may be each independently represented by one of Formulae 3-1 to 3-37, but are not limited thereto:

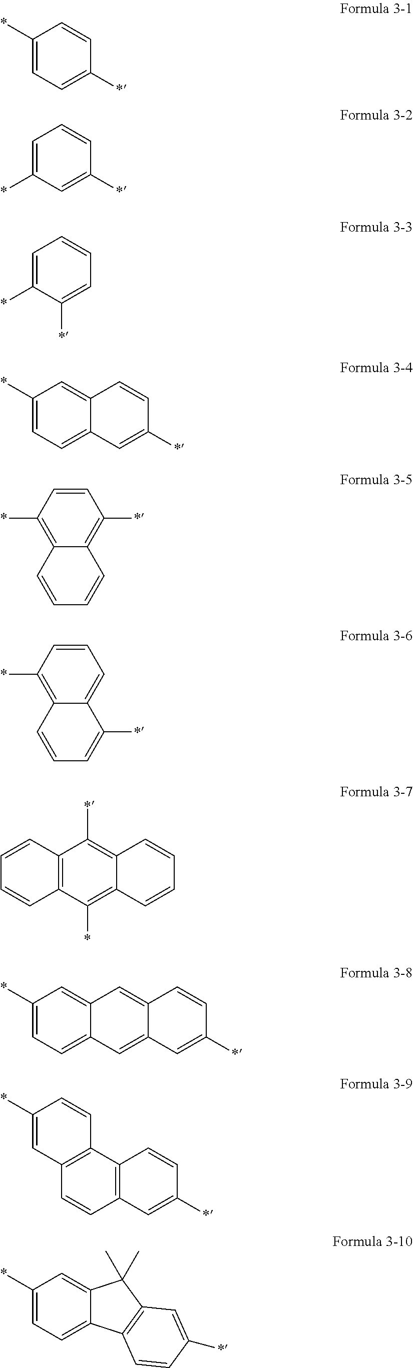

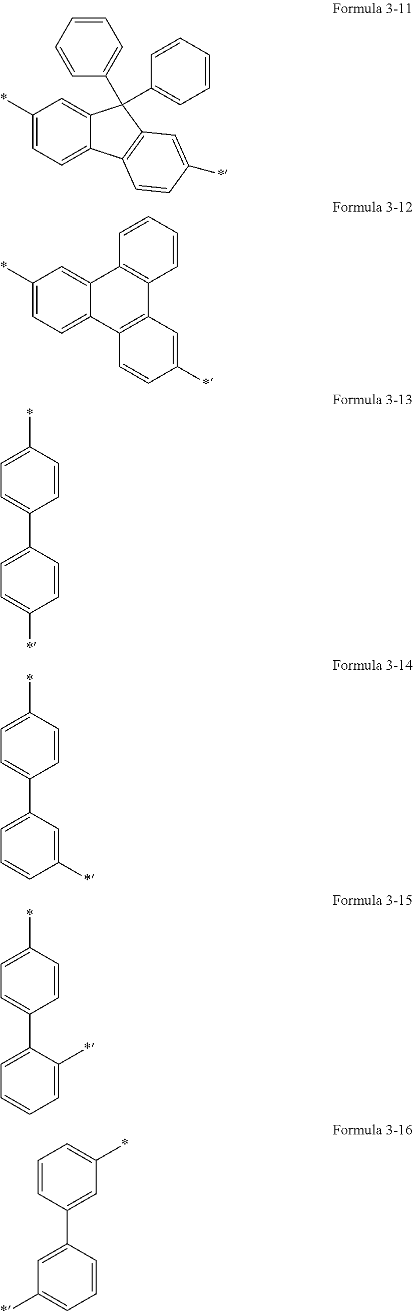

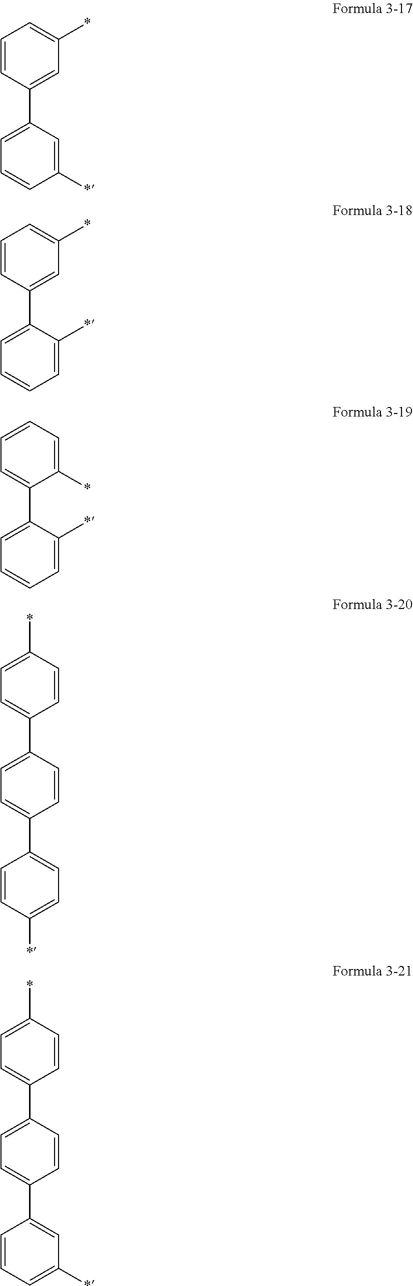

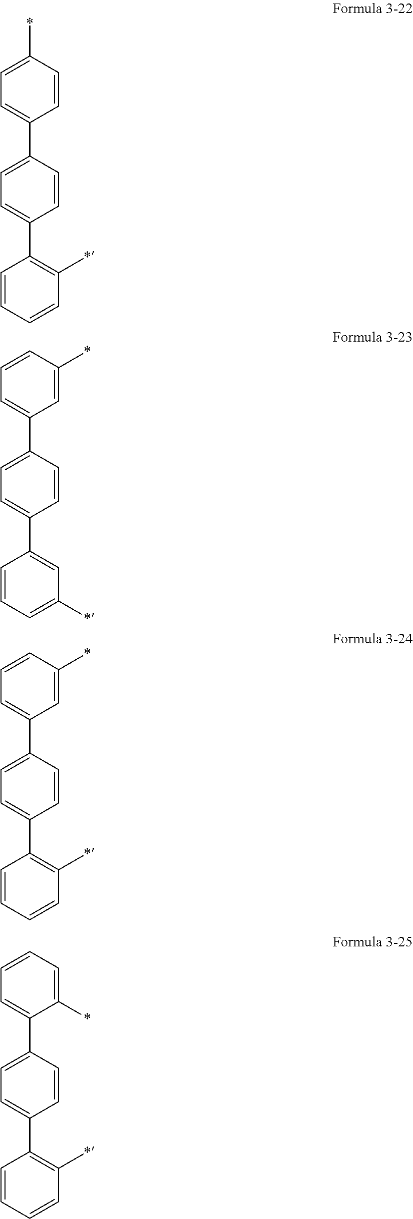

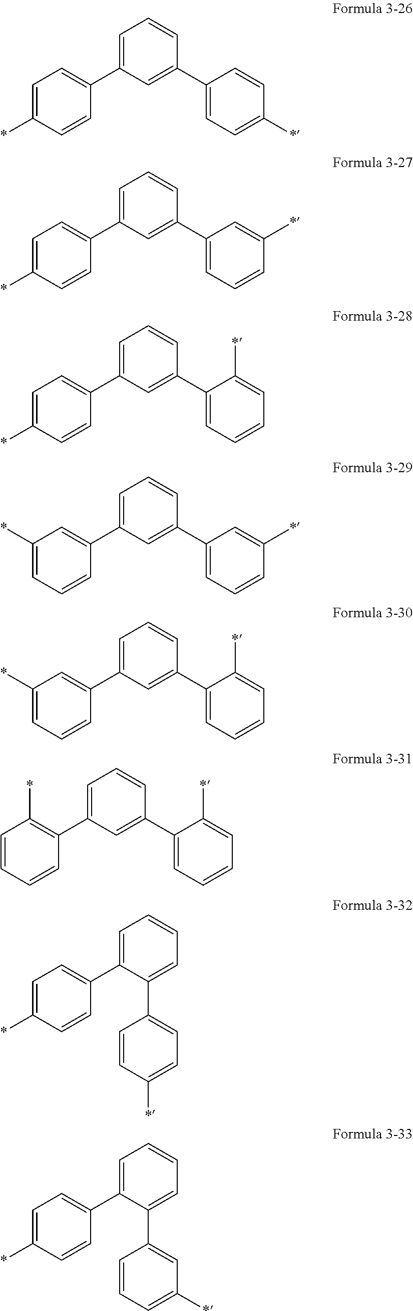

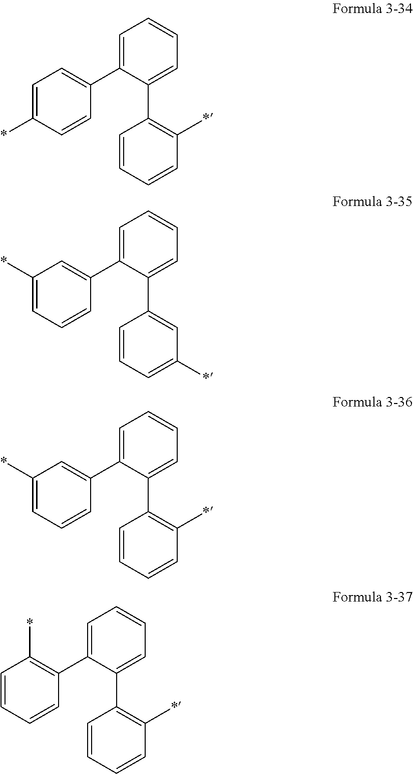

In Formula 1 above, a1, which indicates the number of L₁s, may be 0, 1, 2, 3, 4, or 5, and in some embodiments, 0, 1, or 2, and in some other embodiments, 0 or 1. When a1 is 0, *-(L₁)ₐ₁-*′ may be a single bond. When a1 is 2 or greater, the at least two L₁s may be identical to or different from each other. a2 and a3 in Formula 1 may be may be understood based on the description of a1 and the structure of Formula 1.

In some embodiments, a1, a2, and a3 may be each independently 0, 1, or 2. In above Formulae, R₁ to R₅ may be each independently selected from a hydrogen, a deuterium, a fluoro group (—F), a chloro group (—Cl), a bromo group (—Br), an iodo group (—I), a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₆-C₆₀ aryl group, a substituted or unsubstituted C₆-C₆₀ aryloxy group, a substituted or unsubstituted C₆-C₆₀ arylthio group, and a substituted or unsubstituted monovalent non-aromatic condensed polycyclic group, wherein at least one of R₂ and R₃ is selected from a substituted or unsubstituted C₆-C₆₀ aryl group, and a substituted or unsubstituted monovalent non-aromatic condensed polycyclic group.

In some embodiments, in above Formulae, R₁ to R₅ may be each independently selected from

a hydrogen, a deuterium, —F, —Cl, —Br, —I, a hydroxyl group, a C₁-C₂₀ alkyl group, and a C₁-C₂₀ alkoxy group,

a C₁-C₂₀ alkyl group and a C₁-C₂₀ alkoxy group, each substituted with at least one of a deuterium atom, —F, —Cl, —Br, —I, and a hydroxyl group,

a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, and a ovalenyl group,

a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluorantenyl group, a triphenylenyl group, a pyrenyl group, a chrysenyl group, a naphthacenyl group, a pycenyl group, a perylenyl group, a pentaphenyl group, a hexacenyl group, a pentacenyl group, a rubicenyl group, a coronenyl group, and a ovalenyl group, each substituted with at least one selected from a deuterium, —F, —Cl, —Br, —I, a hydroxyl group, a C₁-C₂₀ alkyl group, a C₁-C₂₀ alkoxy group, —Si(Q₃₃)(Q₃₄)(Q₃₅), a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluorantenyl group, a triphenylenyl group, a pyrenyl group, a chrysenyl group, a naphthacenyl group, a pycenyl group, a perylenyl group, a pentaphenyl group, a hexacenyl group, a pentacenyl group, a rubicenyl group, a coronenyl group, and a ovalenyl group,

wherein i) at least one of R₂ and R₃, and ii) R₁ may be each independently selected from

a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, and a ovalenyl group;

a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluorantenyl group, a triphenylenyl group, a pyrenyl group, a chrysenyl group, a naphthacenyl group, a pycenyl group, a perylenyl group, a pentaphenyl group, a hexacenyl group, a pentacenyl group, a rubicenyl group, a coronenyl group, and a ovalenyl group, each substituted with at least one selected from a deuterium, —F, —Cl, —Br, a hydroxyl group, a C₁-C₂₀ alkyl group, a C₁-C₂₀ alkoxy group, -a phenyl group, a biphenyl group, a terphenyl group, a quaterphenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluorantenyl group, a triphenylenyl group, a pyrenyl group, a chrysenyl group, a naphthacenyl group, a pycenyl group, a perylenyl group, a pentaphenyl group, a hexacenyl group, a pentacenyl group, a rubicenyl group, a coronenyl group, and a ovalenyl group;

In some other embodiments, in Formula 1, 1-1, and 1-2, R₁ to R₅ may be each independently selected from

a hydrogen, a deuterium, —F, —Cl, —Br, —I, a hydroxyl group, a C₁-C₂₀ alkyl group, and a C₁-C₂₀ alkoxy group;

a C₁-C₂₀ alkyl group and a C₁-C₂₀ alkoxy group, each substituted with at least one of a deuterium, —F, —Cl, —Br, or a hydroxyl group;

a phenyl group, a biphenyl group, a terphenyl group, a quaterphenyl group, a naphthyl group, a phenalenyl group, a phenanthrenyl group, an anthracenyl group, a fluorantenyl group, a triphenylenyl group, a pyrenyl group, a chrysenyl group, fluorenyl group, and a perylenyl group;

a phenyl group, a biphenyl group, a terphenyl group, a quaterphenyl group, a naphthyl group, a phenalenyl group, a phenanthrenyl group, an anthracenyl group, a fluorantenyl group, a triphenylenyl group, a pyrenyl group, a chrysenyl group, fluorenyl group, and a perylenyl group, each substituted with at least one selected from a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, a phenalenyl group, a phenanthrenyl group, an anthracenyl group, a fluorantenyl group, a triphenylenyl group, a pyrenyl group, a chrysenyl group, a fluorenyl group, and a perylenyl group; and

i) at least one of R₂ and R₃, and ii) R₁ may be each independently selected from

a phenyl group, a biphenyl group, a terphenyl group, a quaterphenyl group, a naphthyl group, a phenalenyl group, a phenanthrenyl group, an anthracenyl group, a fluoranthenyl group, a triphenylenyl group, a pyrenyl group, a chrysenyl group, a fluorenyl group, and a perylenyl group; or

a phenyl group, a biphenyl group, a terphenyl group, a quaterphenyl group, a naphthyl group, a phenalenyl group, a phenanthrenyl group, an anthracenyl group, a fluorantenyl group, a triphenylenyl group, a pyrenyl group, a chrysenyl group, a fluorenyl group, and a perylenyl group, each substituted with at least one selected from a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, a phenalenyl group, a phenanthrenyl group, an anthracenyl group, a fluorantenyl group, a triphenylenyl group, a pyrenyl group, a chrysenyl group, a fluorenyl group, and a perylenyl group.

In some other embodiments, in Formulae 1, 1-1, and 1-2, R₁ to R₅ may be each independently selected from

a hydrogen, a deuterium, —F, —Cl, —Br, —I, a hydroxyl group, a C₁-C₂₀ alkyl group, and a C₁-C₂₀ alkoxy group;

a C₁-C₂₀ alkyl group and a C₁-C₂₀ alkoxy group, each substituted with at least one of a deuterium, —F, —Cl, —Br, —I, or a hydroxyl group; and

a group represented by one of Formulae 4-1 to 4-5, and 4-34 to 4-37; and

i) at least one of R₂ and R₃, and ii) R₁ may be each independently a group represented by one of Formulae 4-1 to 4-5, and 4-34 to 4-37,

According to another embodiment, in the condensed cyclic compound of the present disclosure, X₁ is S or O,

R₁ to R₅ are each independently hydrogen, a deuterium, —F, —Cl, —Br, —I, a hydroxyl group, a C₁-C₂₀ alkyl group or a C₁-C₂₀ alkoxy group;

a C₁-C₂₀ alkyl group and a C₁-C₂₀ alkoxy group, each substituted with at least one of a deuterium atom, —F, —Cl, —Br, —I, or a hydroxyl group; or

one of the following Formulae 4-1 to 4-5, and 4-34 to 4-37;

at least one of R₂ and R₃ is each independently represented by one of the following Formulae 4-1 to 4-5, and 4-34 to 4-37:

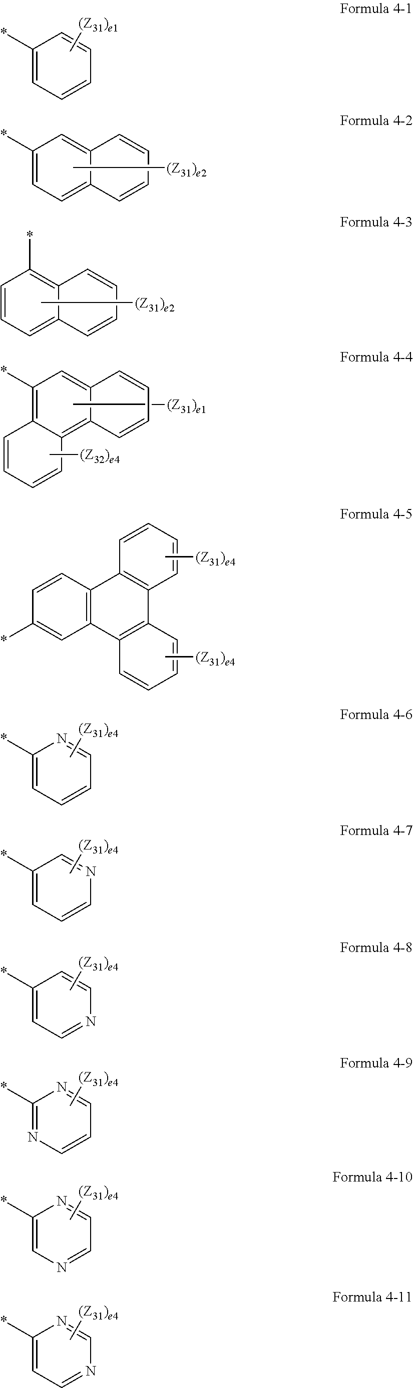

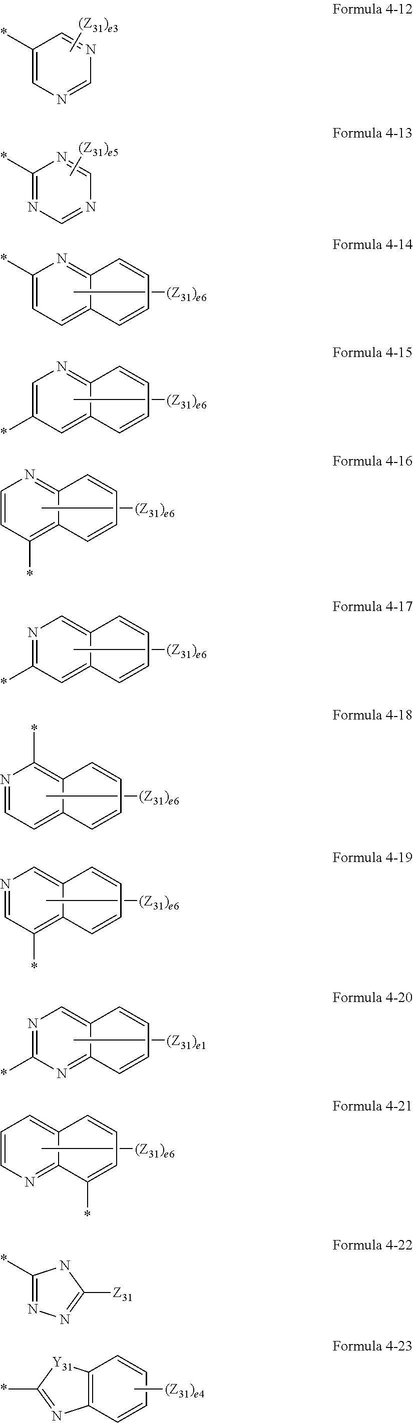

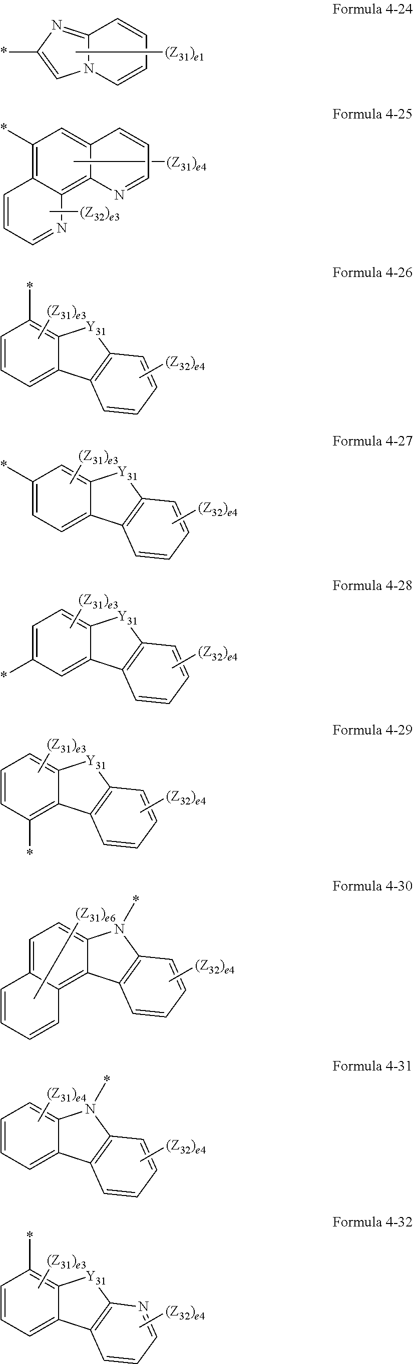

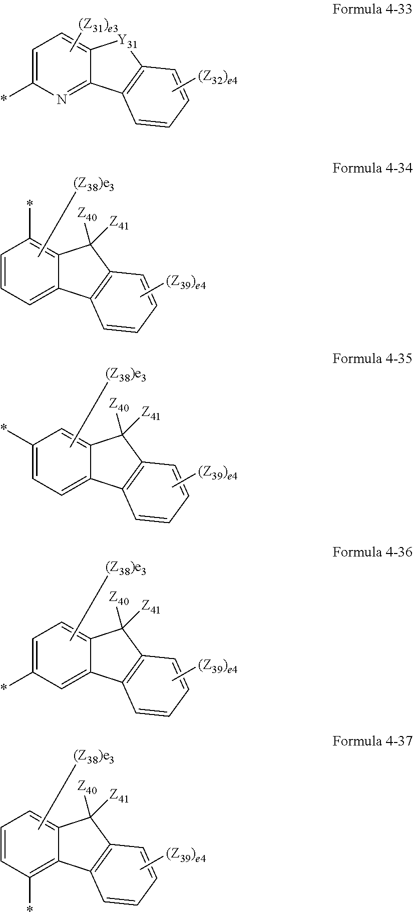

In Formulae 4-1 to 4-37,

Y₃₁ may be 0, S, C(Z₃₃)(Z₃₄), N(Z₃₅), or Si(Z₃₆)(Z₃₇), where Y₃₁ in Formula 4-23 may be not NH,

Z₃₁ to Z₃₇ may be each independently selected from a hydrogen, a deuterium, —F, —Cl, —Br, —I, a hydroxyl group, a cyano group, an amino group, an amidino groups, a C₁-C₂₀ alkyl group, a C₁-C₂₀ alkoxy group, a phenyl group, a naphthyl group, an anthracenyl group, a phenanthrenyl group, a fluorenyl group, a chrysenyl group, a benzocarbazolyl group, a dibenzocarbazolyl group, a dibenzofuranyl group, a dibenzothiophenyl group, a pyridinyl group, a pyrimidinyl group, a carbazolyl group, a triazinyl group, a quinolinyl group, an isoquinolinyl group, a quinazolinyl group, a quinoxalinyl group, a biphenyl group, a terphenyl group, and a quaterphenyl group,

Z₃₈ to Z₄₁ may be each independently selected from a hydrogen, a deuterium, —F, —Cl, —Br, —I, a hydroxyl group, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a chrysenyl group, a biphenyl group, a terphenyl group, and a quaterphenyl group,

e1 may be an integer selected from 1 to 5, e2 may be an integer selected from 1 to 7, e3 may be an integer selected from 1 to 3, e4 may be an integer selected from 1 to 4, e6 may be an integer selected from 1 to 6, and * may be a binding site with an adjacent atom.

In some other embodiments, in Formulae 1, 1-1, and 1-2, R₁ may be selected from

a phenyl group, a biphenyl group, a terphenyl group, a quaterphenyl group, a naphthyl group, a phenalenyl group, a phenanthrenyl group, an anthracenyl group, a fluoranthenyl group, a fluorenyl group, a triphenylenyl group, a pyrenyl group, a chrysenyl group, and a perylenyl group, and

a phenyl group, a biphenyl group, a terphenyl group, a quaterphenyl group, a naphthyl group, a phenalenyl group, a phenanthrenyl group, an anthracenyl group, a fluorantenyl group, a fluorenyl group, a triphenylenyl group, a pyrenyl group, a chrysenyl group, and a perylenyl group, each substituted with at least one selected from a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, a phenalenyl group, a phenanthrenyl group, an anthracenyl group, a fluorantenyl group, a fluorenyl group, a triphenylenyl group, a pyrenyl group, a chrysenyl group, and a perylenyl group.

In some other embodiments, at least one of R₂ and R₃ in above Formulae may be selected from

a phenyl group, a biphenyl group, a terphenyl group, a quaterphenyl group, a naphthyl group, a phenanthrenyl group, an anthracenyl group, a fluoranthenyl group, a fluorenyl group, and a triphenylenyl group, and

a phenyl group, a biphenyl group, a terphenyl group, a quaterphenyl group, a naphthyl group, a phenanthrenyl group, an anthracenyl group, a fluoranthenyl group, a fluorenyl group, and a triphenylenyl group, each substituted with at least one selected from a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, a phenalenyl group, a phenanthrenyl group, an anthracenyl group, a fluorantenyl group, and a triphenylenyl group.

In above Formulae, R₁₁ to R₁₄ may be each independently selected from a hydrogen, a deuterium, —F, —Cl, —Br, —I, a hydroxyl group, a substituted or unsubstituted C₁-C₆₀ alkyl group, a substituted or unsubstituted C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group,

In some embodiments, R₁₁ to R₁₄ in above Formulae may be each independently selected from

a hydrogen, a deuterium, —F, —Cl, —Br, —I, a hydroxyl group, a C₁-C₂₀ alkyl group, and a C₁-C₂₀ alkoxy group,

a C₁-C₂₀ alkyl group and a C₁-C₂₀ alkoxy group, each substituted with at least one of a deuterium atom, —F, —Cl, —Br, —I, or a hydroxyl group,

a phenyl group, a biphenyl group, a terphenyl group, a quaterphenyl group, a naphthyl group, a fluorenyl group, a spiro-fluorenyl group, a benzofluorenyl group, a dibenzofluorenyl group, a phenanthrenyl group, an anthracenyl group, a fluoranthenyl group, a triphenylenyl group, a pyrenyl group, and a chrysenyl group.

In some other embodiments, R₁₁ to R₁₄ in above Formulae may be each independently selected from

a hydrogen, a deuterium, —F, —Cl, —Br, —I, a hydroxyl group, a C₁-C₂₀ alkyl group, and a C₁-C₂₀ alkoxy group,

a phenyl group, a biphenyl group, terphenyl group, quaterphenyl group, a naphthyl group, a fluorenyl group, a spiro-fluorenyl group, a phenanthrenyl group, an anthracenyl group, a fluoranthenyl group, a triphenylenyl group, a pyrenyl group, and a chrysenyl group.

In some other embodiments, in above Formulae, R₁₁ to R₁₄ may be each independently selected from a hydrogen, a deuterium, —F, —Cl, —Br, —I, a hydroxyl group, a C₁-C₂₀ alkyl group, and a C₁-C₂₀ alkoxy group, but are not limited thereto.

In some other embodiments, R₁₁ to R₁₄ in above Formulae may be all hydrogens.

In some other embodiments, R₁ to R₅ in above Formulae may be each independently selected from

a hydrogen, a deuterium, —F, —Cl, —Br, —I, a hydroxyl group, a C₁-C₂₀ alkyl group, and a C₁-C₂₀ alkoxy group,

a C₁-C₂₀ alkyl group and a C₁-C₂₀ alkoxy group, each substituted with at least one of a deuterium atom, —F, —Cl, —Br, —I, or a hydroxyl group, and

a group represented by one of Formulae 5-1 to 5-9, 5-18 to 5-21, and 5-45 to 5-66, and

i) at least one of R₂ and R₃, and ii) R₁ are each independently selected from a group represented by one of Formulae 5-1 to 5-9, 5-18 to 5-21, and 5-45 to 5-66; and

R₁₁ to R₁₄ may be each independently selected from

a hydrogen, a deuterium, —F, —Cl, —Br, —I, a hydroxyl group, a C₁-C₂₀ alkyl group, and a C₁-C₂₀ alkoxy group,

a group represented by one of Formulae 5-1 to 5-9, 5-18 to 5-21, and 5-45 to 5-66, but are not limited thereto,

According to another embodiment, X₁ is S or O, R₁ to R₅ are each independently

hydrogen, a deuterium, —F, —Cl, —Br, —I, a hydroxyl group, a C₁-C₂₀ alkyl group or C₁-C₂₀ alkoxy group;

a C₁-C₂₀ alkyl group and a C₁-C₂₀ alkoxy group, each substituted with at least one of a deuterium atom, —F, —Cl, —Br, —I, or a hydroxyl group; or

one of the following Formulae 5-1 to 5-9, 5-18 to 5-21, and 5-45 to 5-66;

at least one of R₂ and R₃ is each independently represented by one of the following Formulae 5-1 to 5-9, 5-18 to 5-21, and 5-45 to 5-66,

R₁₁ to R₁₄ are each independently

hydrogen, a deuterium, —F, —Cl, —Br, —I, a hydroxyl group, C₁-C₂₀ alkyl group or C₁-C₂₀ alkoxy group; or

one of the following Formulae 5-1 to 5-9, 5-18 to 5-21, and 5-45 to 5-66:

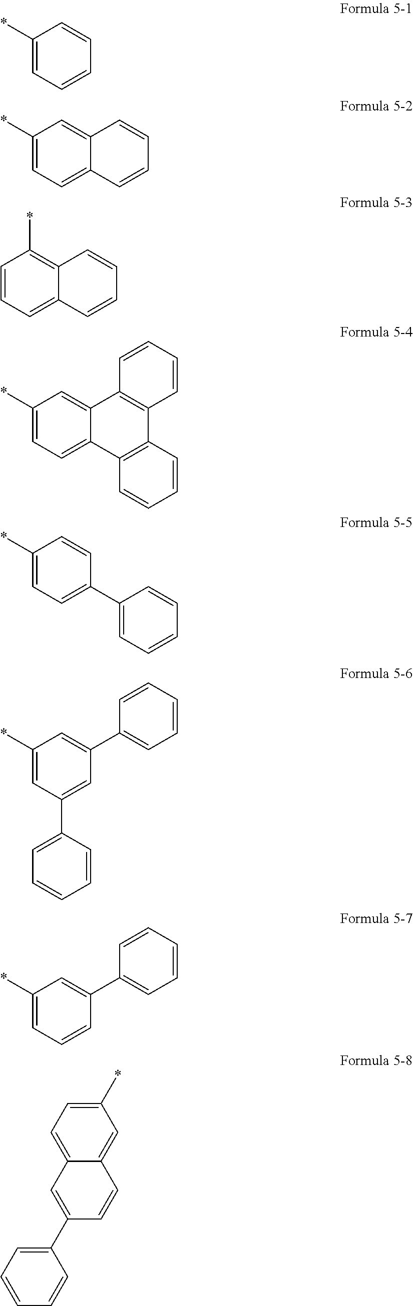

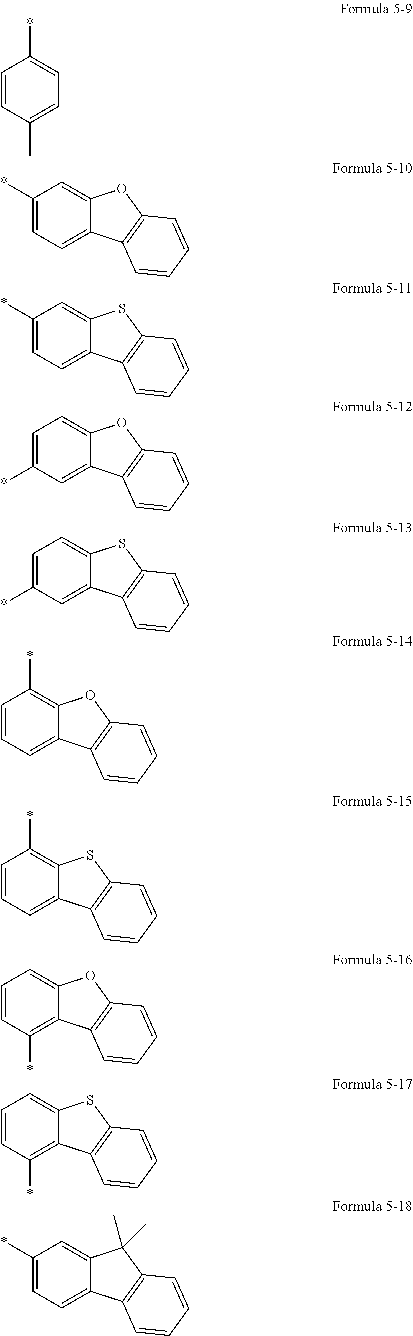

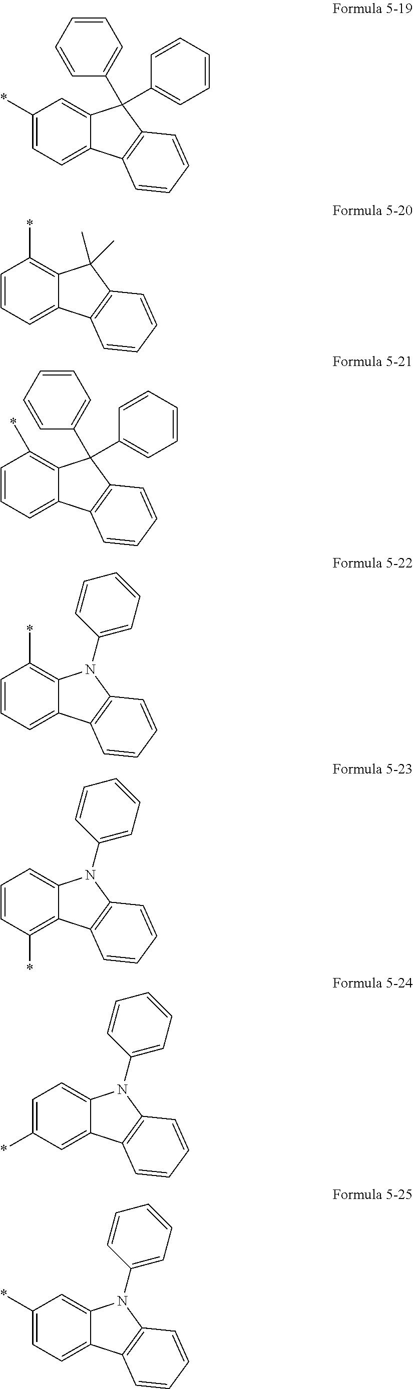

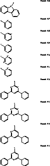

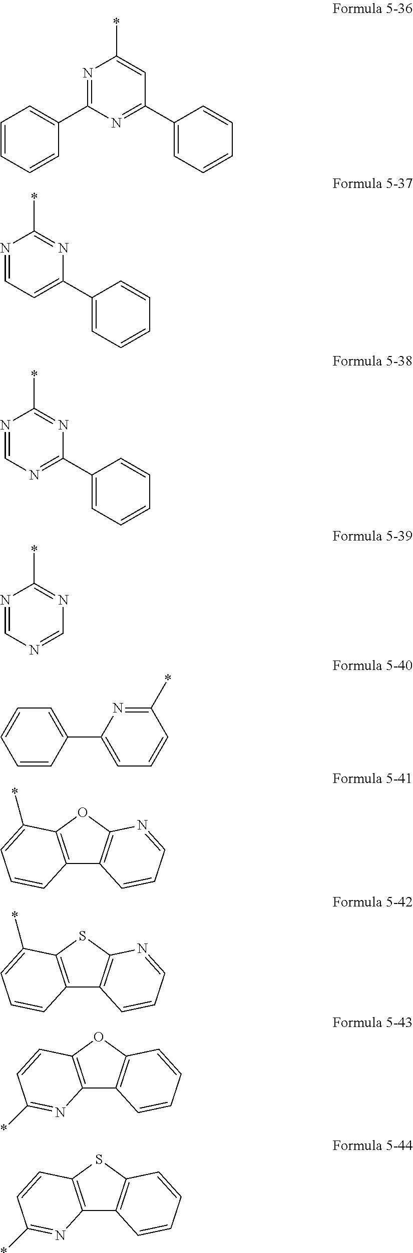

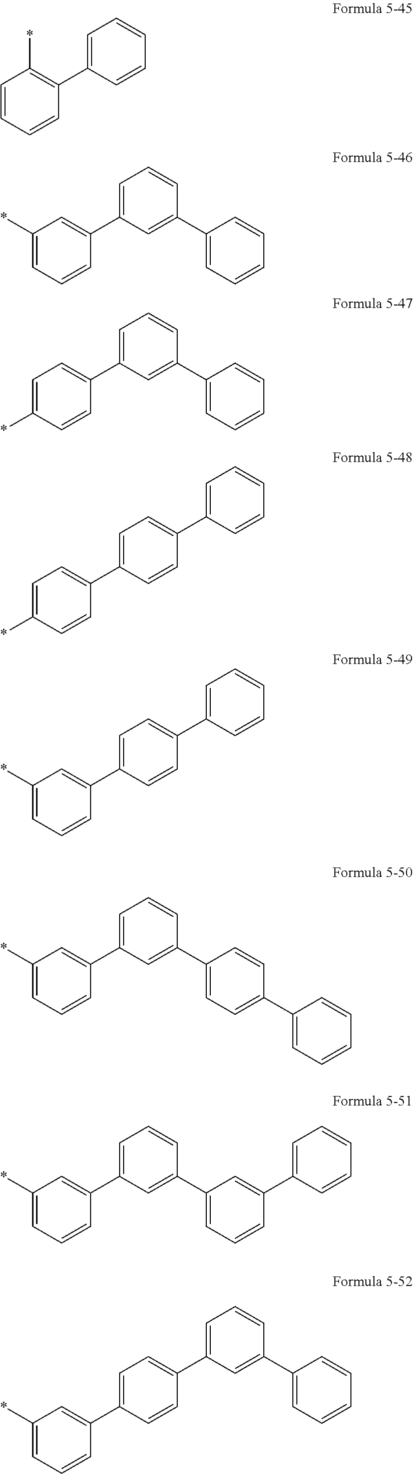

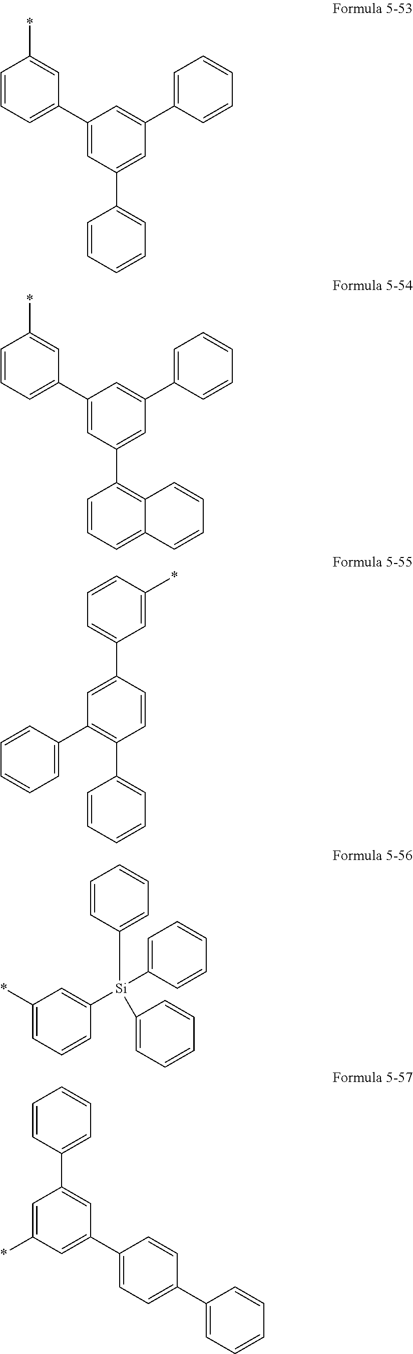

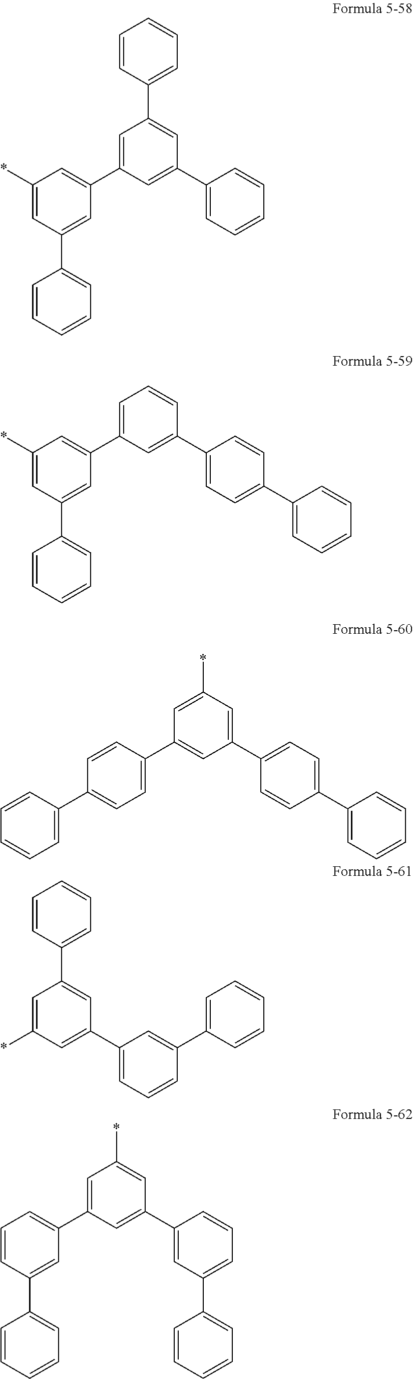

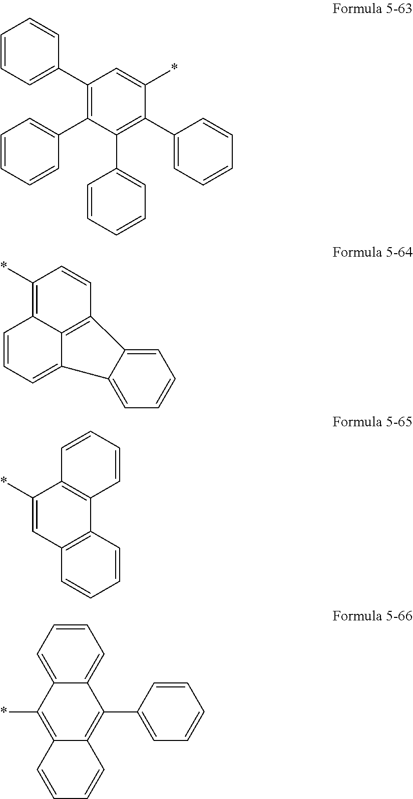

When R₂ in Formula 1 may be a substituted or unsubstituted phenyl group, R₃ is selected from a hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted fluoranthenyl group, a substituted or unsubstituted triphenylenyl group, a substituted or unsubstituted pyrenyl group, a substituted or unsubstituted phenanthrenyl group, a substituted or unsubstituted fluorenyl group, and a substituted or unsubstituted chrysenyl group.

In Formula 1 above, b1, which indicates the number of R₁s, may be an integer of 1 to 3, and in some embodiments, may be 1 or 2. For example, b1 may be 1. When b1 is 2 or greater, the at least two R₁ may be identical to or different from each other. b2 and b3 in Formula 1 may be may be understood based on the description of b1 and the structure of Formula 1.

In some embodiments, in any of the formulae herein, at least one of substituents of the substituted C₆-C₆₀ arylene group may be selected from

a deuterium, —F, —Cl, —Br, —I, a hydroxyl group, a C₁-C₆₀ alkyl group, and a C₁-C₆₀ alkoxy group,

a C₁-C₆₀ alkyl group, and a C₁-C₆₀ alkoxy group, each substituted with at least one of a deuterium, —F, —Cl, —Br, —I, and a hydroxyl group,

a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group,

a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, each substituted with at least one of a deuterium, —F, —Cl, —Br, —I, a hydroxyl group, a C₁-C₆₀ alkyl group, a C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group.

In some other embodiments, in any of the formulae herein, at least one of substituents of the substituted C₆-C₆₀ arylene group may be selected from

a C₁-C₆₀ alkyl group, and a C₁-C₆₀ alkoxy group, each substituted with at least one of a deuterium, —F, —Cl, —Br, —I, a hydroxyl group, a naphthyl group, a fluorenyl group, a spiro-fluorenyl group, a benzofluorenyl group, a dibenzofluorenyl group, a phenanthrenyl group, an anthracenyl group, a fluoranthenyl group, a triphenylenyl group, a pyrenyl group, and a chrysenyl group,

a phenyl group, a biphenyl group, a terphenyl group, a quaterphenyl group, a naphthyl group, a fluorenyl group, a spiro-fluorenyl group, a benzofluorenyl group, a dibenzofluorenyl group, a phenanthrenyl group, an anthracenyl group, a fluoranthenyl group, a triphenylenyl group, a pyrenyl group, and a chrysenyl group,

a phenyl group, a biphenyl group, a terphenyl group, a quaterphenyl group, a naphthyl group, a fluorenyl group, a spiro-fluorenyl group, a benzofluorenyl group, a dibenzofluorenyl group, a phenanthrenyl group, an anthracenyl group, a fluoranthenyl group, a triphenylenyl group, a pyrenyl group, and a chrysenyl group, each substituted with at least one of a deuterium, —F, —Cl, —Br, —I, a hydroxyl group, a C₁-C₆₀ alkyl group, a C₁-C₆₀ alkoxy group, a phenyl group, a biphenyl group, a terphenyl group, a quaterphenyl group, a naphthyl group, a fluorenyl group, a spiro-fluorenyl group, a benzofluorenyl group, a dibenzofluorenyl group, a phenanthrenyl group, an anthracenyl group, a fluoranthenyl group, a triphenylenyl group, a pyrenyl group, and a chrysenyl group,

In some embodiments, the condensed cyclic compound of Formula 1 above may be one of Compounds below, but is not limited thereto:

### [Group □]

Group of X₁=S in Formula 1-1

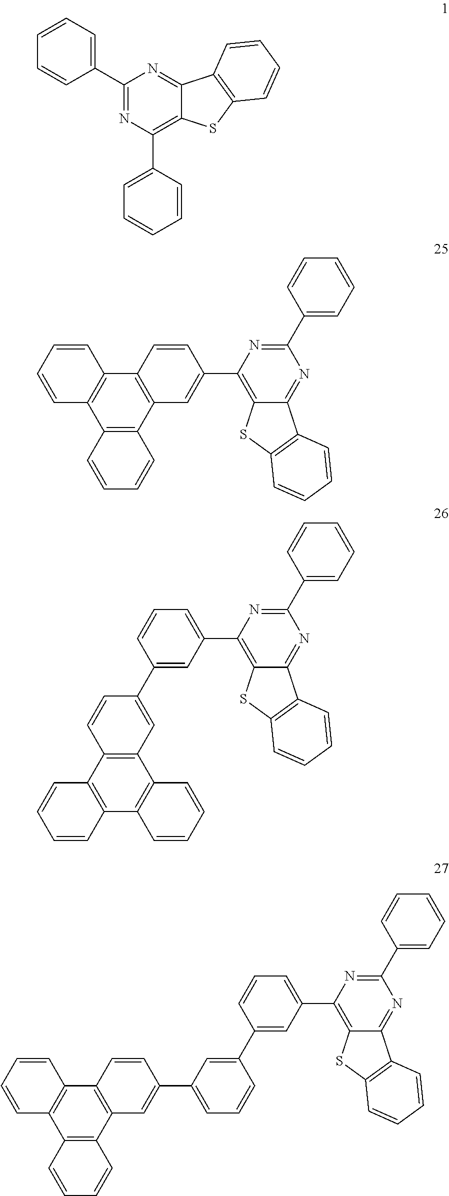

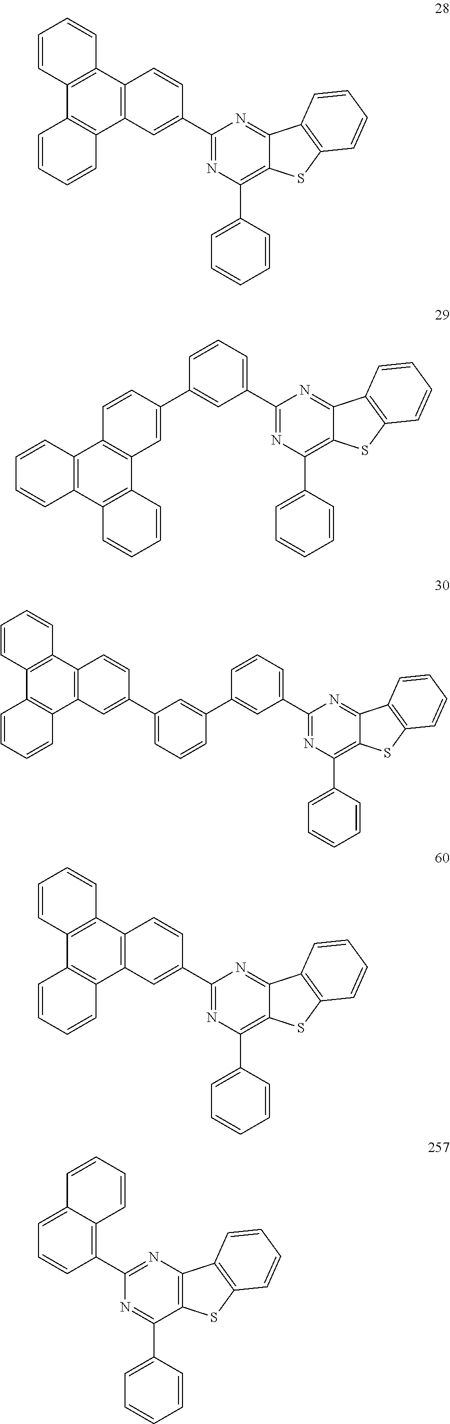 [SMILES: c1ccc(-c2nc(-c3ccc4c5ccccc5c5ccccc5c4c3)nc3c2sc2ccccc23)cc1 ; c1ccc(-c2nc(-c3cccc(-c4ccc5c6ccccc6c6ccccc6c5c4)c3)nc3c2sc2ccccc23)cc1 ; c1ccc(-c2nc(-c3cccc(-c4cccc(-c5ccc6c7ccccc7c7ccccc7c6c5)c4)c3)nc3c2sc2ccccc23)cc1 ; c1ccc(-c2nc(-c3ccc4c5ccccc5c5ccccc5c4c3)nc3c2sc2ccccc23)cc1 ; c1ccc(-c2nc(-c3cccc4ccccc34)nc3c2sc2ccccc23)cc1]

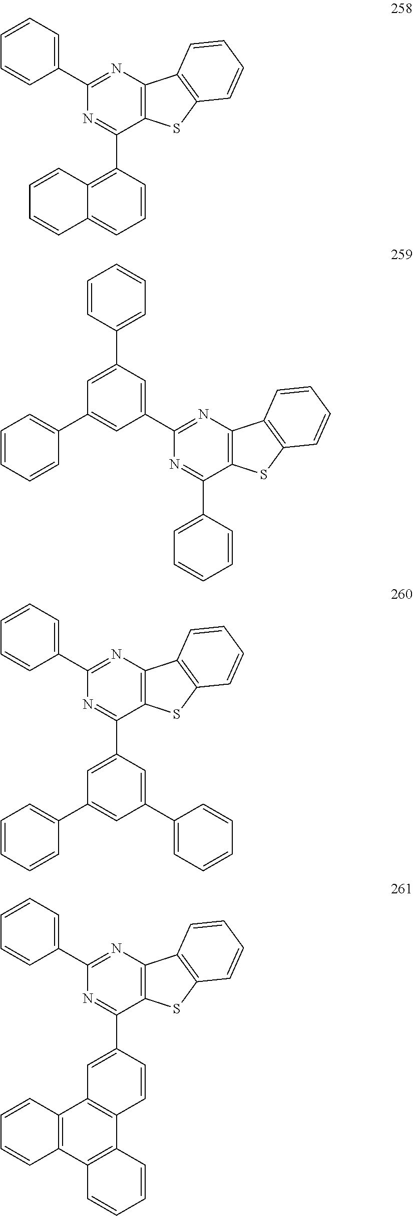

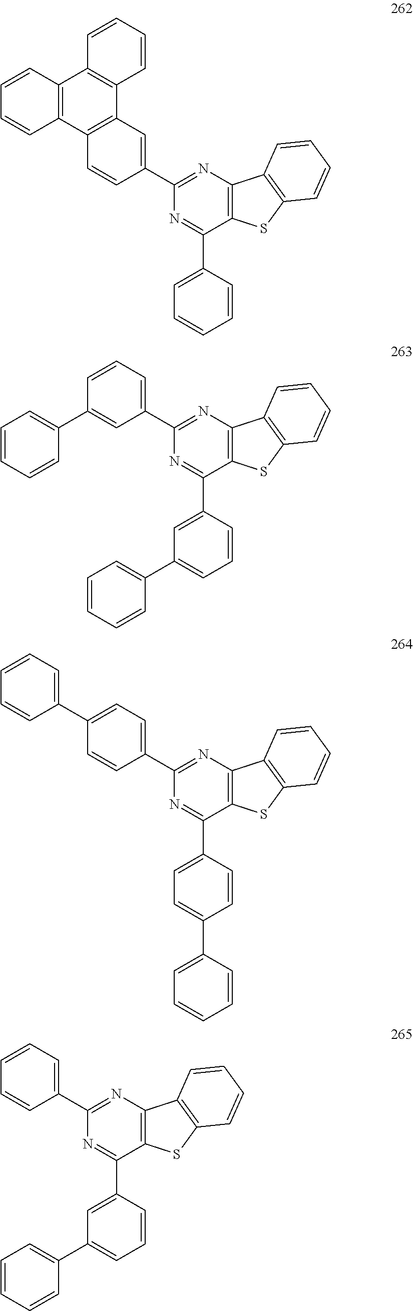 [SMILES: c1ccc(-c2nc(-c3ccc4c5ccccc5c5ccccc5c4c3)nc3c2sc2ccccc23)cc1 ; c1ccc(-c2cccc(-c3nc(-c4cccc(-c5ccccc5)c4)c4sc5ccccc5c4n3)c2)cc1 ; c1ccc(-c2ccc(-c3nc(-c4ccc(-c5ccccc5)cc4)c4sc5ccccc5c4n3)cc2)cc1 ; c1ccc(-c2cccc(-c3nc(-c4ccccc4)nc4c3sc3ccccc34)c2)cc1]

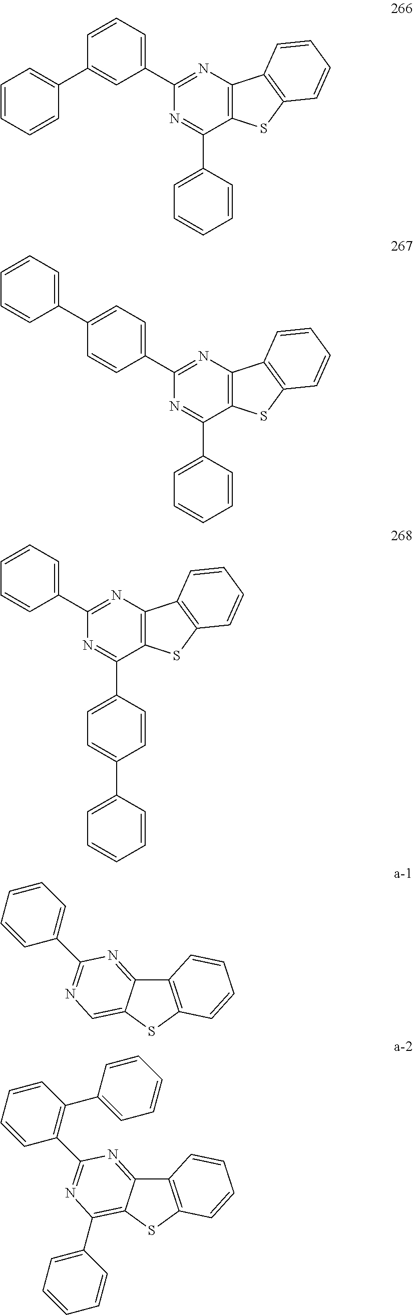

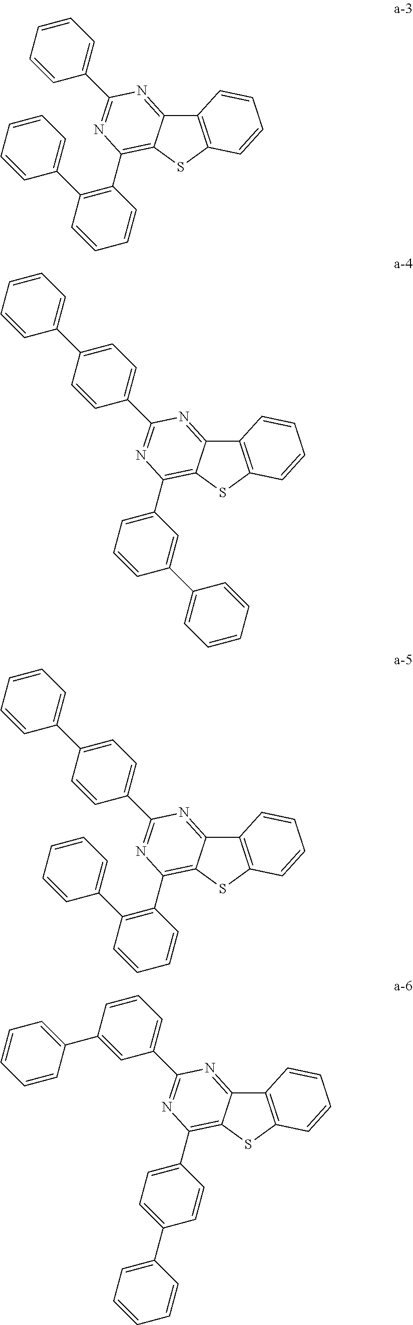

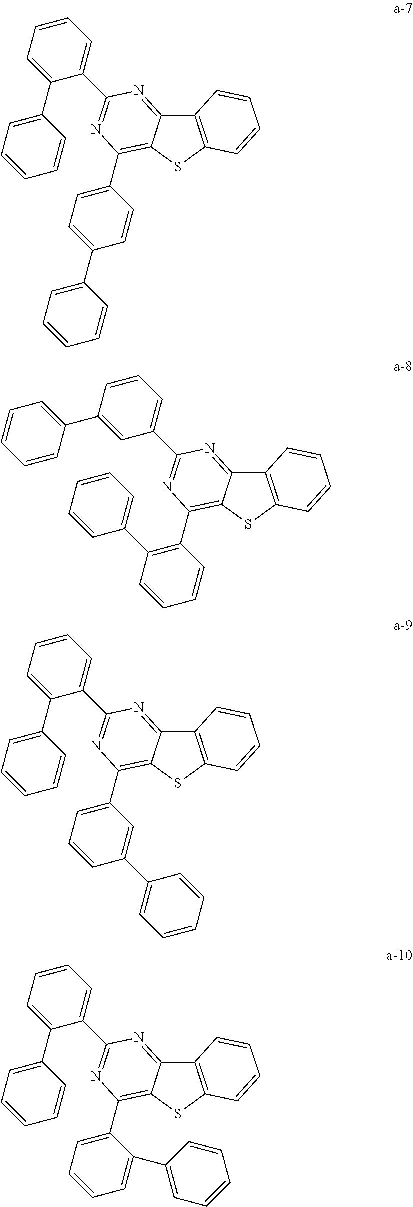

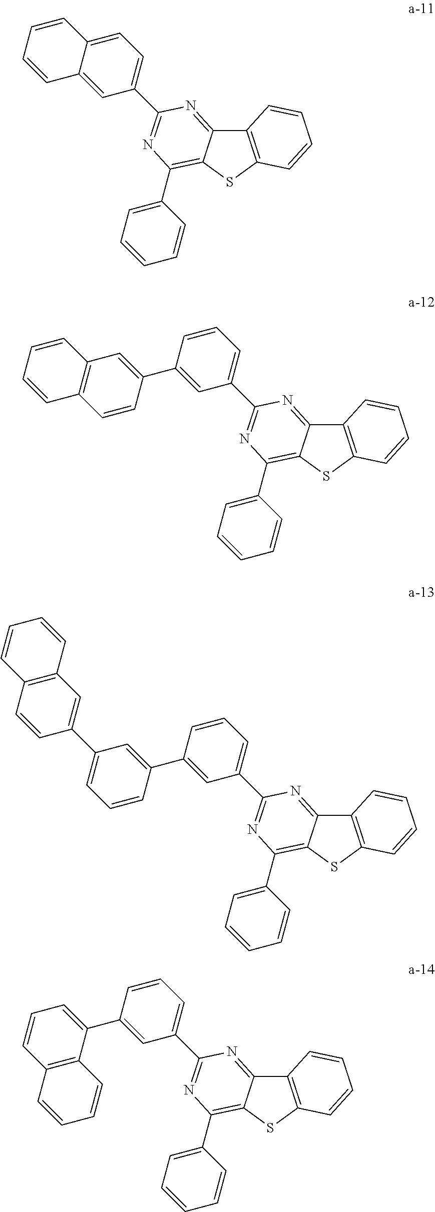 [SMILES: c1ccc(-c2nc(-c3ccc4ccccc4c3)nc3c2sc2ccccc23)cc1 ; c1ccc(-c2nc(-c3cccc(-c4ccc5ccccc5c4)c3)nc3c2sc2ccccc23)cc1 ; c1ccc(-c2nc(-c3cccc(-c4cccc(-c5ccc6ccccc6c5)c4)c3)nc3c2sc2ccccc23)cc1 ; c1ccc(-c2nc(-c3cccc(-c4cccc5ccccc45)c3)nc3c2sc2ccccc23)cc1]

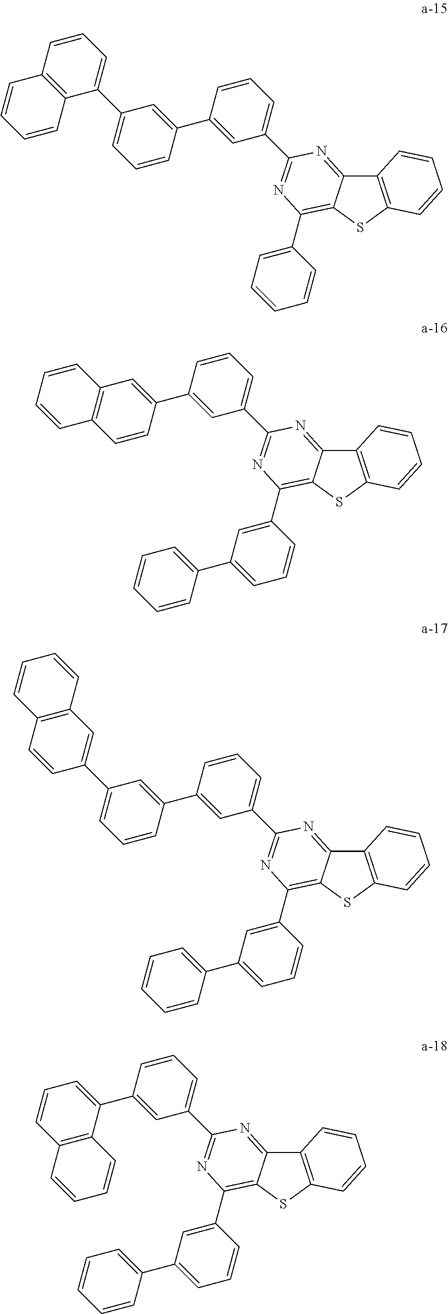

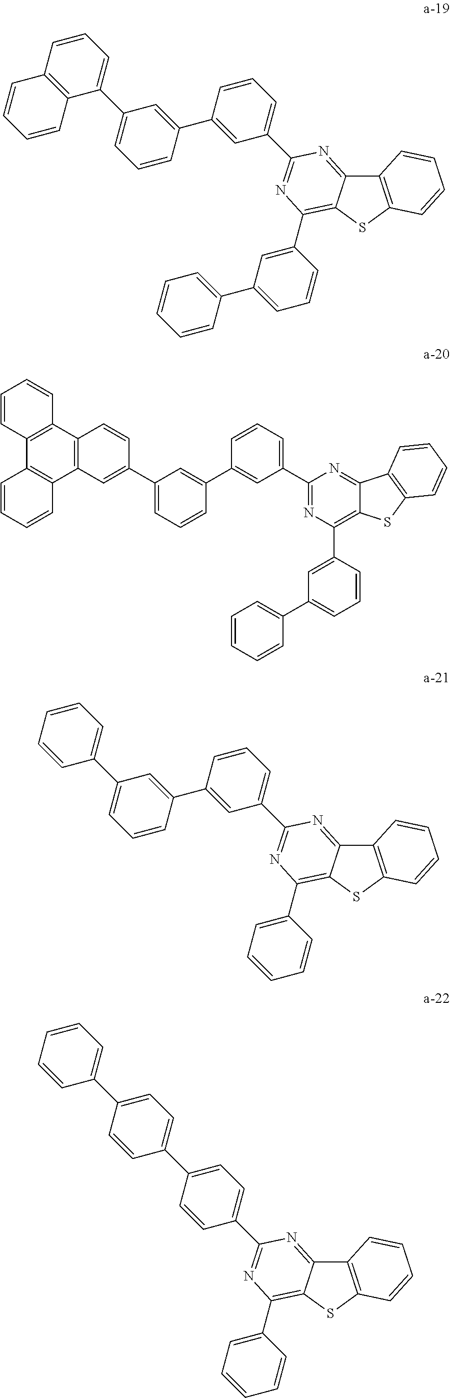 [SMILES: c1ccc(-c2cccc(-c3nc(-c4cccc(-c5cccc(-c6cccc7ccccc67)c5)c4)nc4c3sc3ccccc34)c2)cc1 ; c1ccc(-c2cccc(-c3nc(-c4cccc(-c5cccc(-c6ccc7c8ccccc8c8ccccc8c7c6)c5)c4)nc4c3sc3ccccc34)c2)cc1 ; c1ccc(-c2cccc(-c3cccc(-c4nc(-c5ccccc5)c5sc6ccccc6c5n4)c3)c2)cc1 ; c1ccc(-c2ccc(-c3ccc(-c4nc(-c5ccccc5)c5sc6ccccc6c5n4)cc3)cc2)cc1]

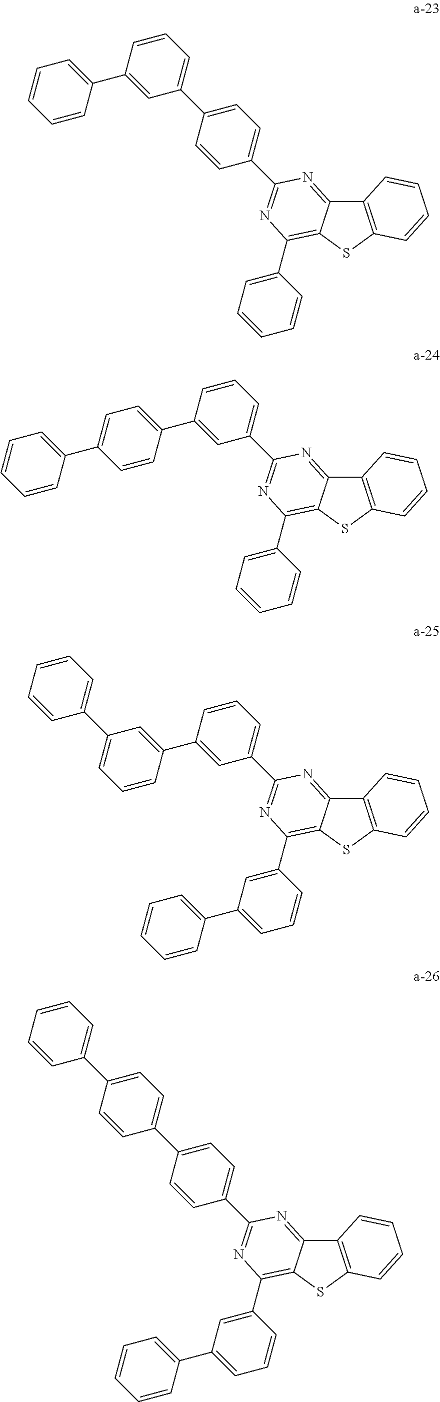 [SMILES: c1ccc(-c2cccc(-c3ccc(-c4nc(-c5ccccc5)c5sc6ccccc6c5n4)cc3)c2)cc1 ; c1ccc(-c2ccc(-c3cccc(-c4nc(-c5ccccc5)c5sc6ccccc6c5n4)c3)cc2)cc1 ; c1ccc(-c2cccc(-c3cccc(-c4nc(-c5cccc(-c6ccccc6)c5)c5sc6ccccc6c5n4)c3)c2)cc1 ; c1ccc(-c2ccc(-c3ccc(-c4nc(-c5cccc(-c6ccccc6)c5)c5sc6ccccc6c5n4)cc3)cc2)cc1]

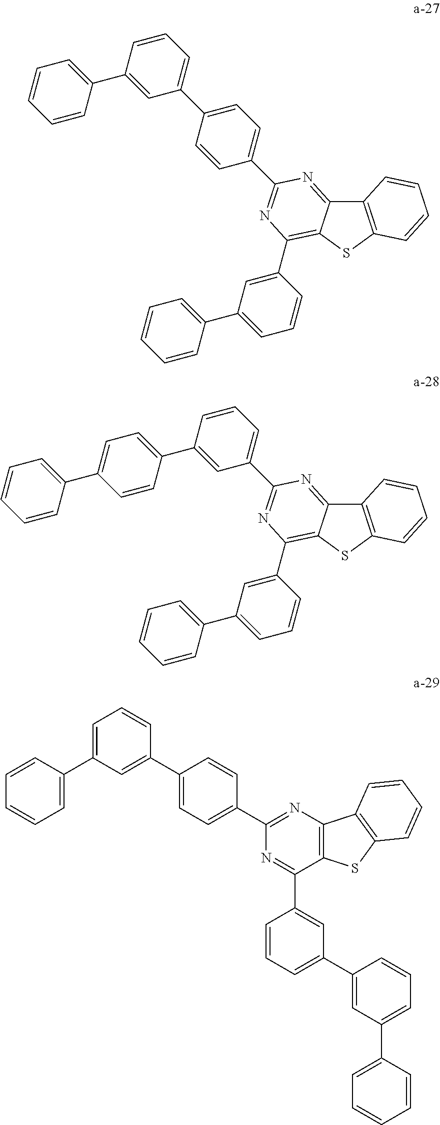 [SMILES: c1ccc(-c2cccc(-c3ccc(-c4nc(-c5cccc(-c6ccccc6)c5)c5sc6ccccc6c5n4)cc3)c2)cc1 ; c1ccc(-c2ccc(-c3cccc(-c4nc(-c5cccc(-c6ccccc6)c5)c5sc6ccccc6c5n4)c3)cc2)cc1 ; c1ccc(-c2cccc(-c3ccc(-c4nc(-c5cccc(-c6cccc(-c7ccccc7)c6)c5)c5sc6ccccc6c5n4)cc3)c2)cc1]

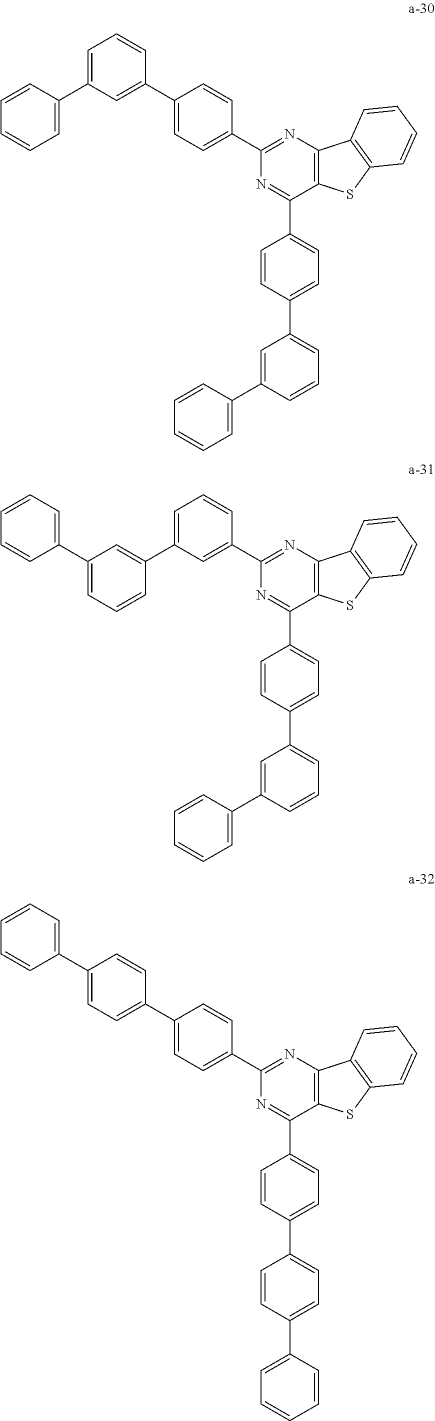

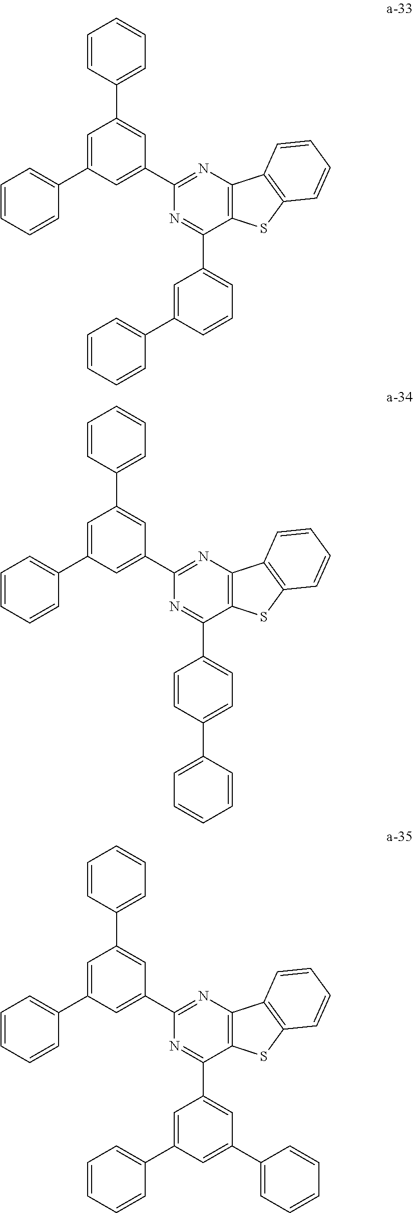

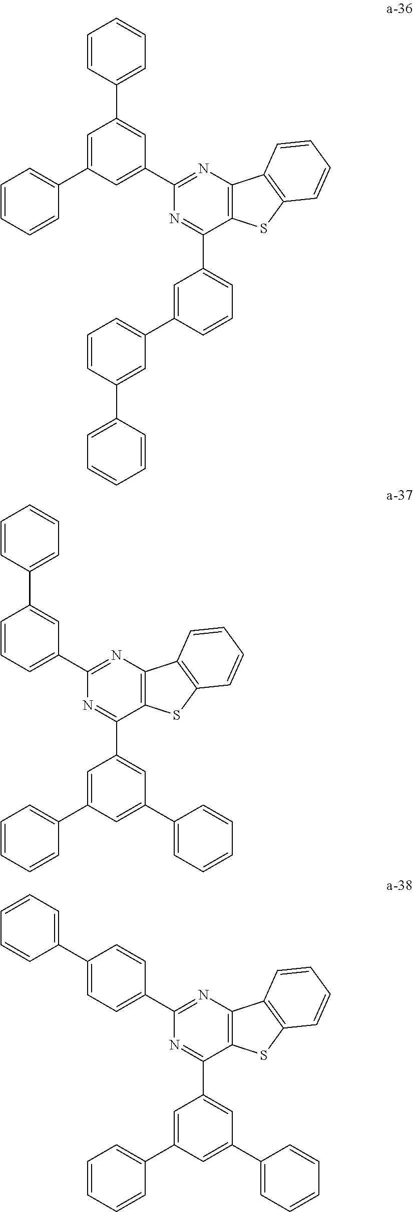

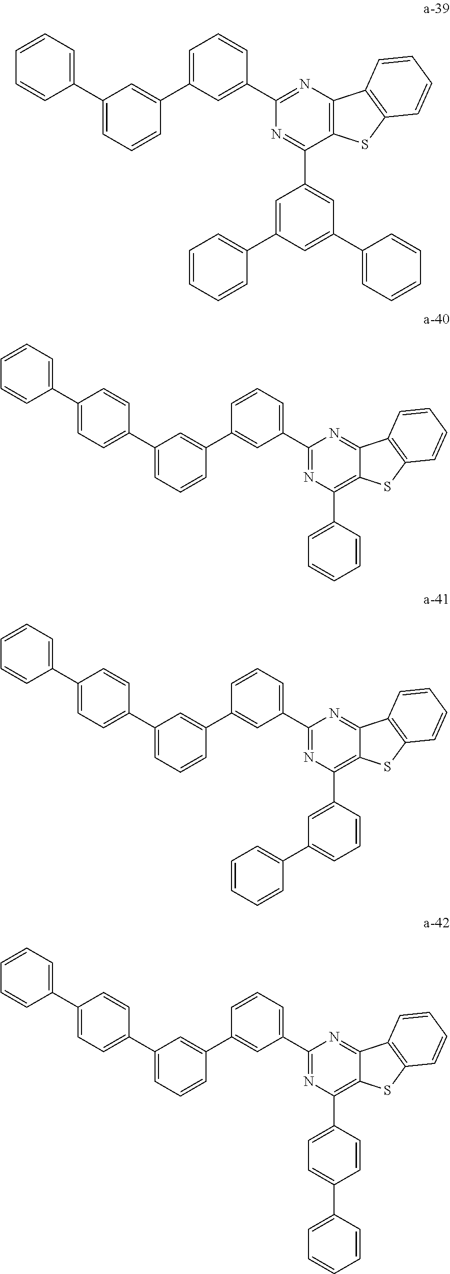 [SMILES: c1ccc(-c2cccc(-c3cccc(-c4nc(-c5cc(-c6ccccc6)cc(-c6ccccc6)c5)c5sc6ccccc6c5n4)c3)c2)cc1 ; c1ccc(-c2ccc(-c3cccc(-c4cccc(-c5nc(-c6ccccc6)c6sc7ccccc7c6n5)c4)c3)cc2)cc1 ; c1ccc(-c2ccc(-c3cccc(-c4cccc(-c5nc(-c6cccc(-c7ccccc7)c6)c6sc7ccccc7c6n5)c4)c3)cc2)cc1 ; c1ccc(-c2ccc(-c3cccc(-c4cccc(-c5nc(-c6ccc(-c7ccccc7)cc6)c6sc7ccccc7c6n5)c4)c3)cc2)cc1]

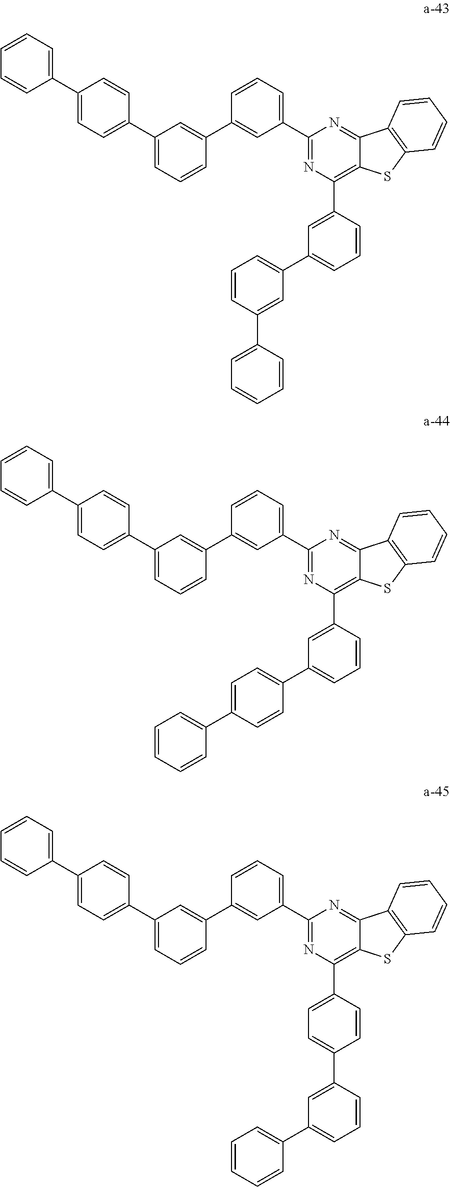 [SMILES: c1ccc(-c2ccc(-c3cccc(-c4cccc(-c5nc(-c6cccc(-c7cccc(-c8ccccc8)c7)c6)c6sc7ccccc7c6n5)c4)c3)cc2)cc1 ; c1ccc(-c2ccc(-c3cccc(-c4cccc(-c5nc(-c6cccc(-c7ccc(-c8ccccc8)cc7)c6)c6sc7ccccc7c6n5)c4)c3)cc2)cc1 ; c1ccc(-c2ccc(-c3cccc(-c4cccc(-c5nc(-c6ccc(-c7cccc(-c8ccccc8)c7)cc6)c6sc7ccccc7c6n5)c4)c3)cc2)cc1]

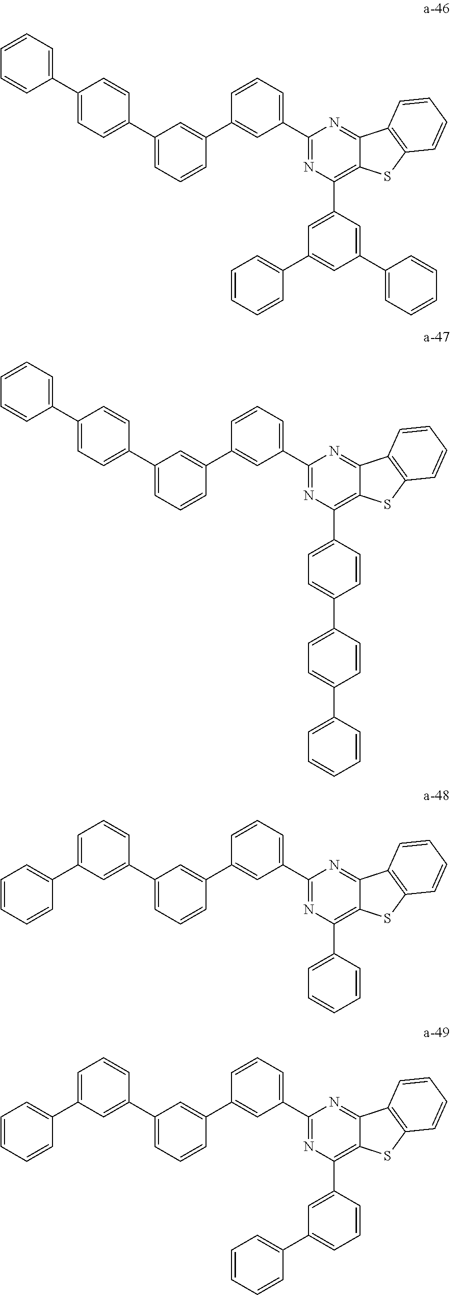 [SMILES: c1ccc(-c2ccc(-c3cccc(-c4cccc(-c5nc(-c6cc(-c7ccccc7)cc(-c7ccccc7)c6)c6sc7ccccc7c6n5)c4)c3)cc2)cc1 ; c1ccc(-c2ccc(-c3ccc(-c4nc(-c5cccc(-c6cccc(-c7ccc(-c8ccccc8)cc7)c6)c5)nc5c4sc4ccccc45)cc3)cc2)cc1 ; c1ccc(-c2cccc(-c3cccc(-c4cccc(-c5nc(-c6ccccc6)c6sc7ccccc7c6n5)c4)c3)c2)cc1 ; c1ccc(-c2cccc(-c3cccc(-c4cccc(-c5nc(-c6cccc(-c7ccccc7)c6)c6sc7ccccc7c6n5)c4)c3)c2)cc1]

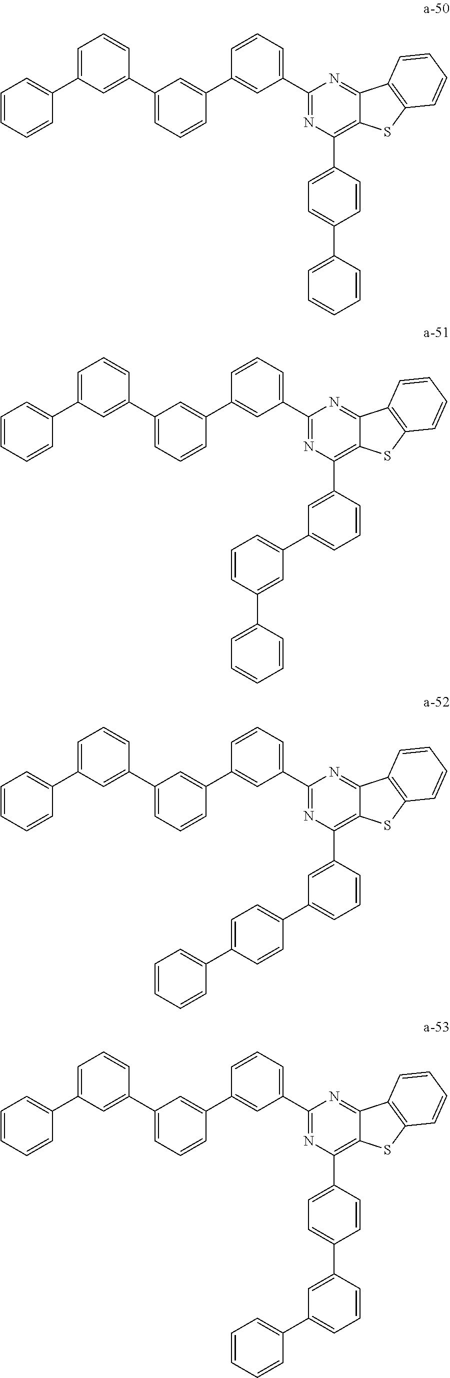 [SMILES: c1ccc(-c2ccc(-c3nc(-c4cccc(-c5cccc(-c6cccc(-c7ccccc7)c6)c5)c4)nc4c3sc3ccccc34)cc2)cc1 ; c1ccc(-c2cccc(-c3cccc(-c4cccc(-c5nc(-c6cccc(-c7cccc(-c8ccccc8)c7)c6)c6sc7ccccc7c6n5)c4)c3)c2)cc1 ; c1ccc(-c2ccc(-c3cccc(-c4nc(-c5cccc(-c6cccc(-c7cccc(-c8ccccc8)c7)c6)c5)nc5c4sc4ccccc45)c3)cc2)cc1 ; c1ccc(-c2cccc(-c3ccc(-c4nc(-c5cccc(-c6cccc(-c7cccc(-c8ccccc8)c7)c6)c5)nc5c4sc4ccccc45)cc3)c2)cc1]

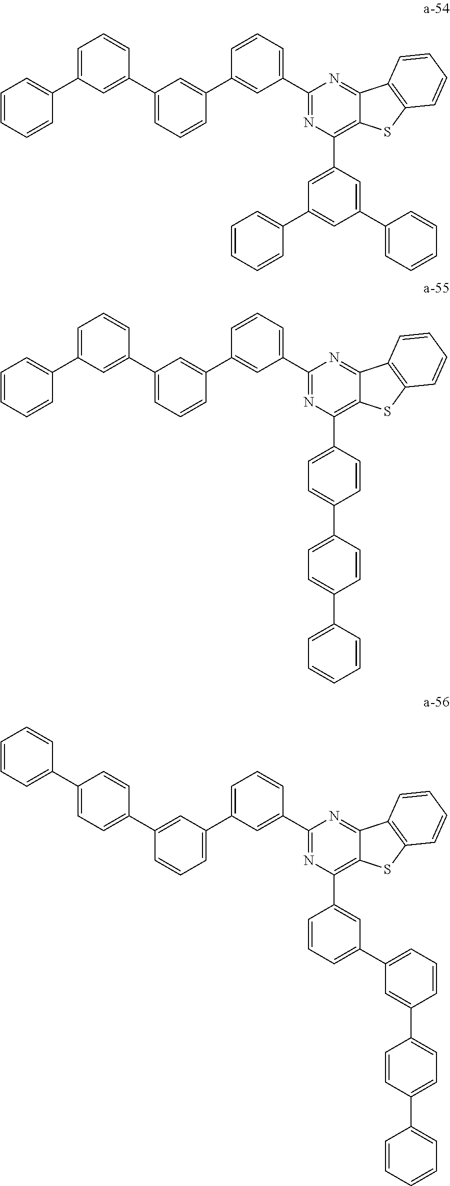 [SMILES: c1ccc(-c2cccc(-c3cccc(-c4cccc(-c5nc(-c6cc(-c7ccccc7)cc(-c7ccccc7)c6)c6sc7ccccc7c6n5)c4)c3)c2)cc1 ; c1ccc(-c2ccc(-c3ccc(-c4nc(-c5cccc(-c6cccc(-c7cccc(-c8ccccc8)c7)c6)c5)nc5c4sc4ccccc45)cc3)cc2)cc1 ; c1ccc(-c2ccc(-c3cccc(-c4cccc(-c5nc(-c6cccc(-c7cccc(-c8ccc(-c9ccccc9)cc8)c7)c6)c6sc7ccccc7c6n5)c4)c3)cc2)cc1]

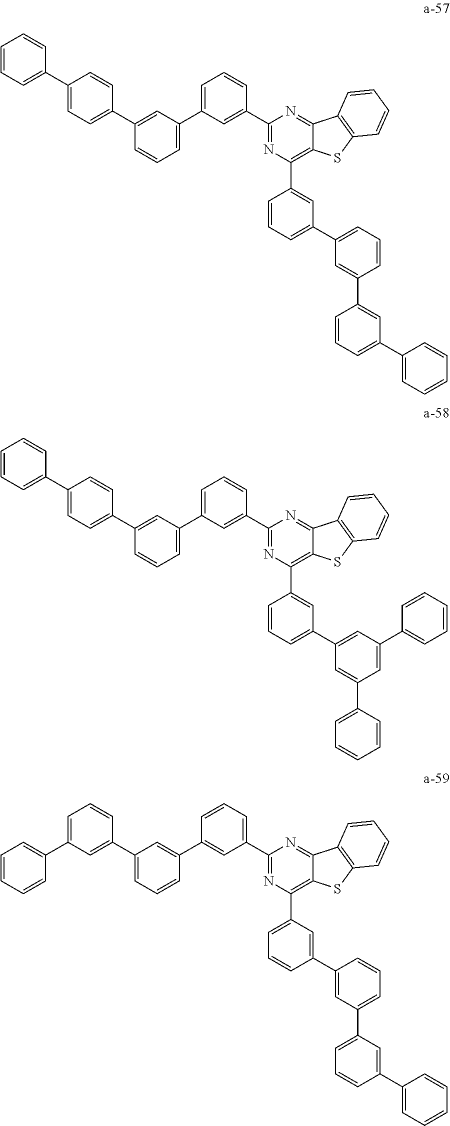 [SMILES: c1ccc(-c2ccc(-c3cccc(-c4cccc(-c5nc(-c6cccc(-c7cccc(-c8cccc(-c9ccccc9)c8)c7)c6)c6sc7ccccc7c6n5)c4)c3)cc2)cc1 ; c1ccc(-c2ccc(-c3cccc(-c4cccc(-c5nc(-c6cccc(-c7cc(-c8ccccc8)cc(-c8ccccc8)c7)c6)c6sc7ccccc7c6n5)c4)c3)cc2)cc1 ; c1ccc(-c2cccc(-c3cccc(-c4cccc(-c5nc(-c6cccc(-c7cccc(-c8cccc(-c9ccccc9)c8)c7)c6)c6sc7ccccc7c6n5)c4)c3)c2)cc1]

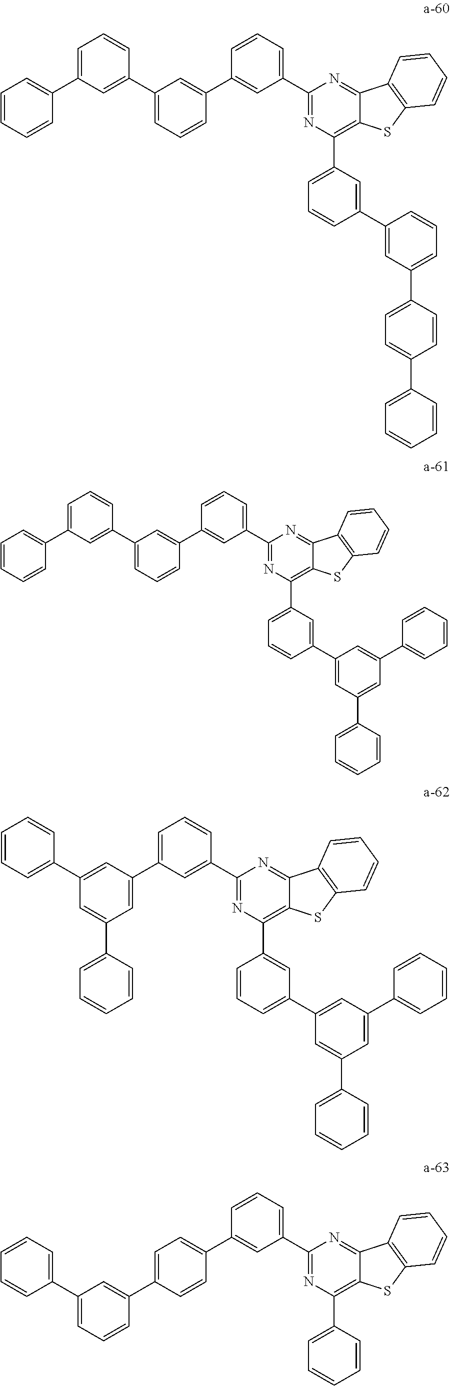 [SMILES: c1ccc(-c2ccc(-c3cccc(-c4cccc(-c5nc(-c6cccc(-c7cccc(-c8cccc(-c9ccccc9)c8)c7)c6)nc6c5sc5ccccc56)c4)c3)cc2)cc1 ; c1ccc(-c2cccc(-c3cccc(-c4cccc(-c5nc(-c6cccc(-c7cc(-c8ccccc8)cc(-c8ccccc8)c7)c6)c6sc7ccccc7c6n5)c4)c3)c2)cc1 ; c1ccc(-c2cc(-c3ccccc3)cc(-c3cccc(-c4nc(-c5cccc(-c6cc(-c7ccccc7)cc(-c7ccccc7)c6)c5)c5sc6ccccc6c5n4)c3)c2)cc1 ; c1ccc(-c2cccc(-c3ccc(-c4cccc(-c5nc(-c6ccccc6)c6sc7ccccc7c6n5)c4)cc3)c2)cc1]

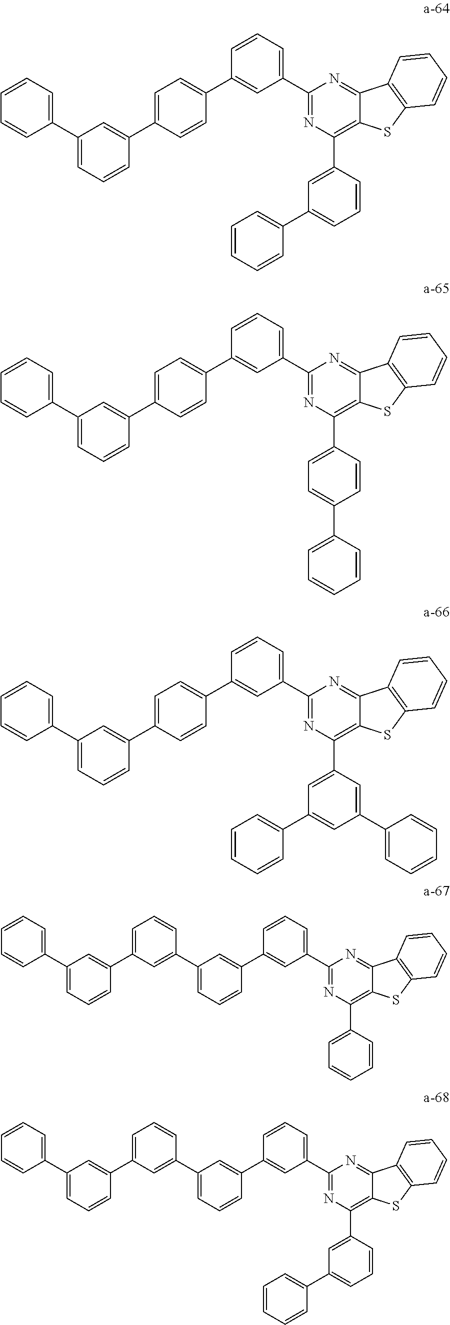 [SMILES: c1ccc(-c2cccc(-c3ccc(-c4cccc(-c5nc(-c6cccc(-c7ccccc7)c6)c6sc7ccccc7c6n5)c4)cc3)c2)cc1 ; c1ccc(-c2ccc(-c3nc(-c4cccc(-c5ccc(-c6cccc(-c7ccccc7)c6)cc5)c4)nc4c3sc3ccccc34)cc2)cc1 ; c1ccc(-c2cccc(-c3ccc(-c4cccc(-c5nc(-c6cc(-c7ccccc7)cc(-c7ccccc7)c6)c6sc7ccccc7c6n5)c4)cc3)c2)cc1 ; c1ccc(-c2cccc(-c3cccc(-c4cccc(-c5cccc(-c6nc(-c7ccccc7)c7sc8ccccc8c7n6)c5)c4)c3)c2)cc1 ; c1ccc(-c2cccc(-c3cccc(-c4cccc(-c5cccc(-c6nc(-c7cccc(-c8ccccc8)c7)c7sc8ccccc8c7n6)c5)c4)c3)c2)cc1]

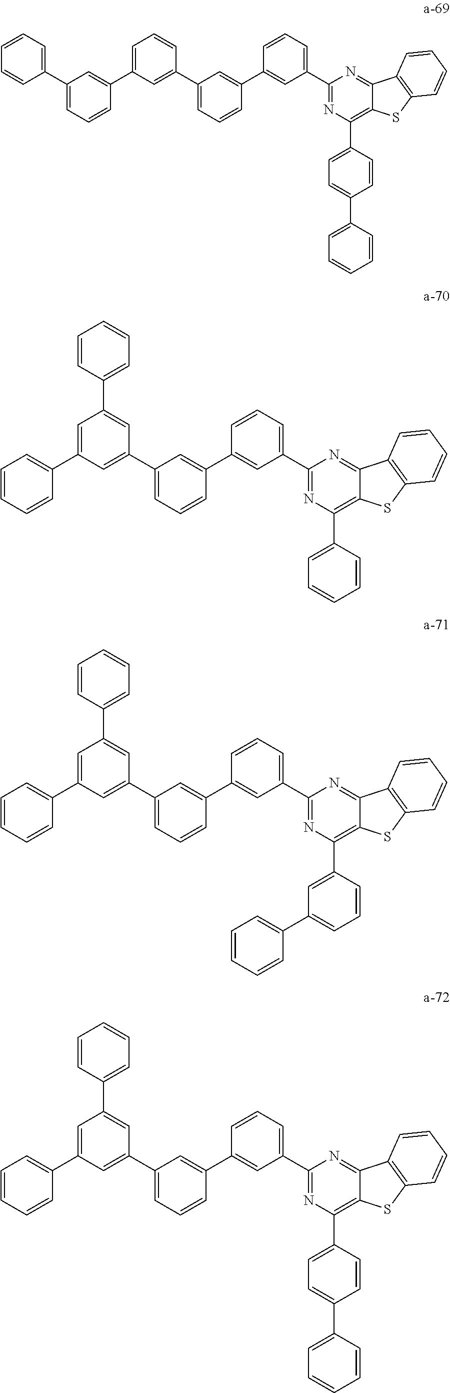 [SMILES: c1ccc(-c2ccc(-c3nc(-c4cccc(-c5cccc(-c6cccc(-c7cccc(-c8ccccc8)c7)c6)c5)c4)nc4c3sc3ccccc34)cc2)cc1 ; c1ccc(-c2cc(-c3ccccc3)cc(-c3cccc(-c4cccc(-c5nc(-c6ccccc6)c6sc7ccccc7c6n5)c4)c3)c2)cc1 ; c1ccc(-c2cc(-c3ccccc3)cc(-c3cccc(-c4cccc(-c5nc(-c6cccc(-c7ccccc7)c6)c6sc7ccccc7c6n5)c4)c3)c2)cc1 ; c1ccc(-c2ccc(-c3nc(-c4cccc(-c5cccc(-c6cc(-c7ccccc7)cc(-c7ccccc7)c6)c5)c4)nc4c3sc3ccccc34)cc2)cc1]

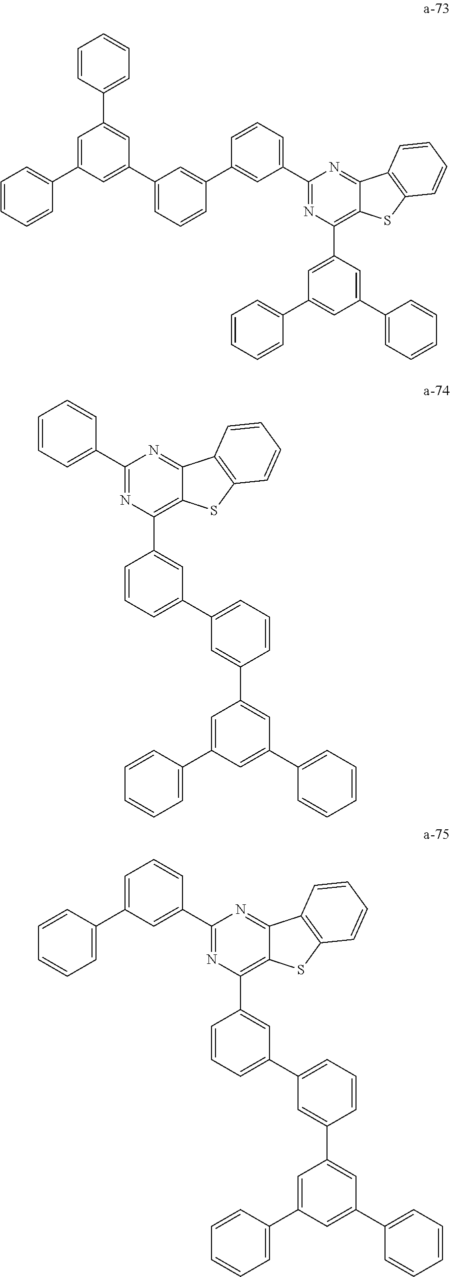

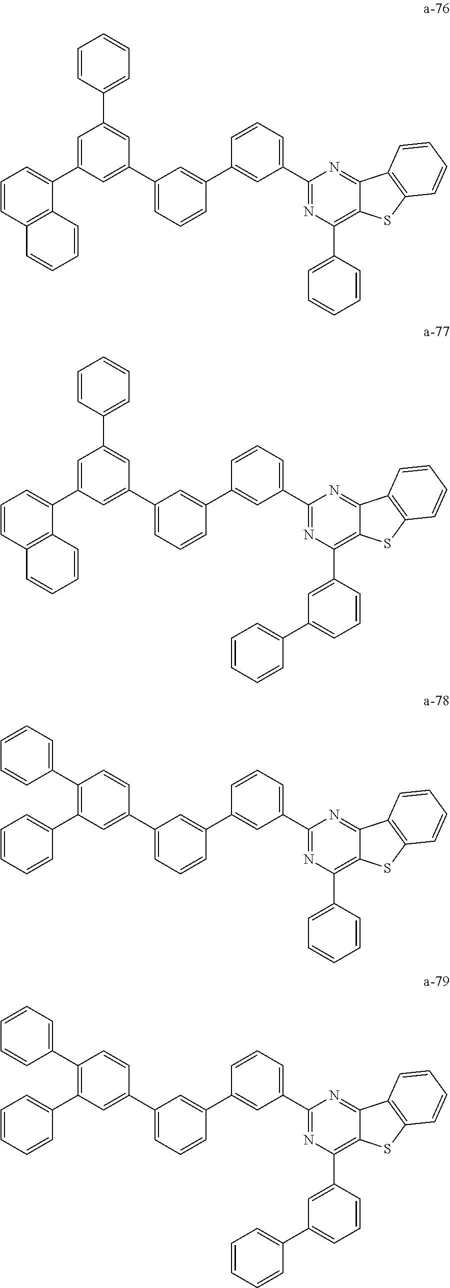 [SMILES: c1ccc(-c2cc(-c3cccc(-c4cccc(-c5nc(-c6ccccc6)c6sc7ccccc7c6n5)c4)c3)cc(-c3cccc4ccccc34)c2)cc1 ; c1ccc(-c2cc(-c3cccc(-c4cccc(-c5nc(-c6cccc(-c7ccccc7)c6)c6sc7ccccc7c6n5)c4)c3)cc(-c3cccc4ccccc34)c2)cc1 ; c1ccc(-c2ccc(-c3cccc(-c4cccc(-c5nc(-c6ccccc6)c6sc7ccccc7c6n5)c4)c3)cc2-c2ccccc2)cc1 ; c1ccc(-c2cccc(-c3nc(-c4cccc(-c5cccc(-c6ccc(-c7ccccc7)c(-c7ccccc7)c6)c5)c4)nc4c3sc3ccccc34)c2)cc1]

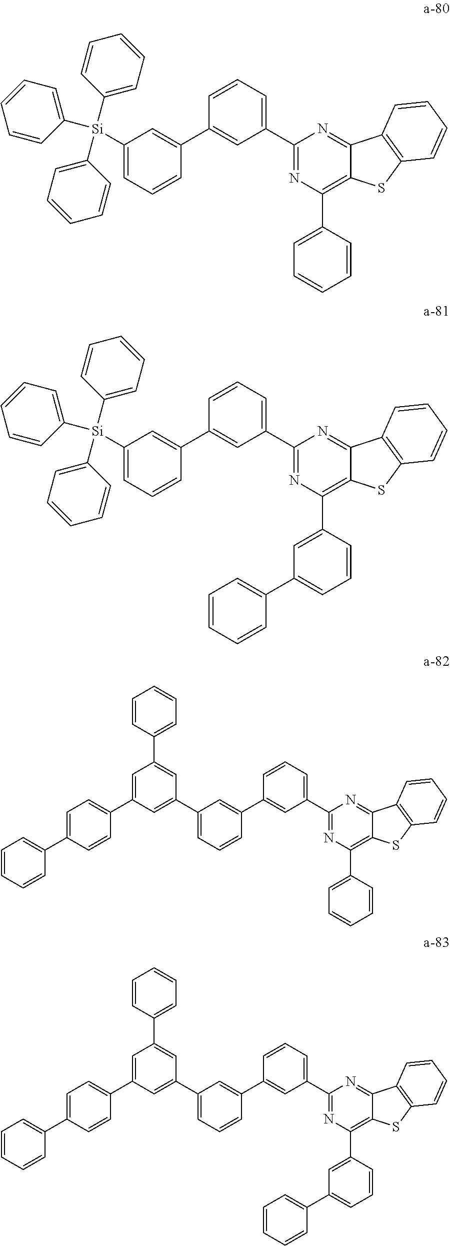 [SMILES: c1ccc(-c2nc(-c3cccc(-c4cccc([Si](c5ccccc5)(c5ccccc5)c5ccccc5)c4)c3)nc3c2sc2ccccc23)cc1 ; c1ccc(-c2cccc(-c3nc(-c4cccc(-c5cccc([Si](c6ccccc6)(c6ccccc6)c6ccccc6)c5)c4)nc4c3sc3ccccc34)c2)cc1 ; c1ccc(-c2ccc(-c3cc(-c4ccccc4)cc(-c4cccc(-c5cccc(-c6nc(-c7ccccc7)c7sc8ccccc8c7n6)c5)c4)c3)cc2)cc1 ; c1ccc(-c2ccc(-c3cc(-c4ccccc4)cc(-c4cccc(-c5cccc(-c6nc(-c7cccc(-c8ccccc8)c7)c7sc8ccccc8c7n6)c5)c4)c3)cc2)cc1]

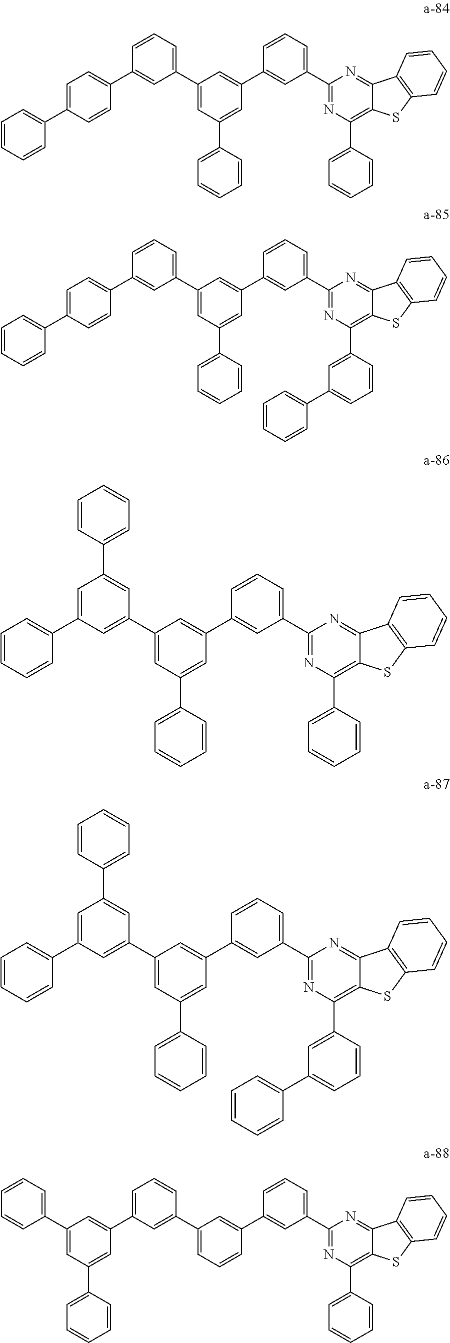

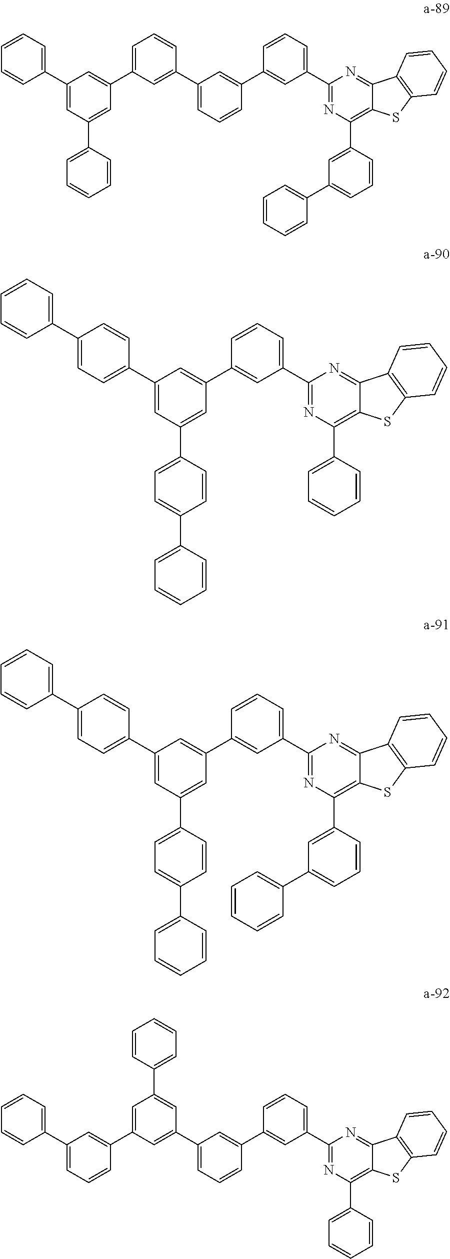 [SMILES: c1ccc(-c2cc(-c3ccccc3)cc(-c3cccc(-c4cccc(-c5cccc(-c6nc(-c7cccc(-c8ccccc8)c7)c7sc8ccccc8c7n6)c5)c4)c3)c2)cc1 ; c1ccc(-c2ccc(-c3cc(-c4ccc(-c5ccccc5)cc4)cc(-c4cccc(-c5nc(-c6ccccc6)c6sc7ccccc7c6n5)c4)c3)cc2)cc1 ; c1ccc(-c2ccc(-c3cc(-c4ccc(-c5ccccc5)cc4)cc(-c4cccc(-c5nc(-c6cccc(-c7ccccc7)c6)c6sc7ccccc7c6n5)c4)c3)cc2)cc1 ; c1ccc(-c2cccc(-c3cc(-c4ccccc4)cc(-c4cccc(-c5cccc(-c6nc(-c7ccccc7)c7sc8ccccc8c7n6)c5)c4)c3)c2)cc1]

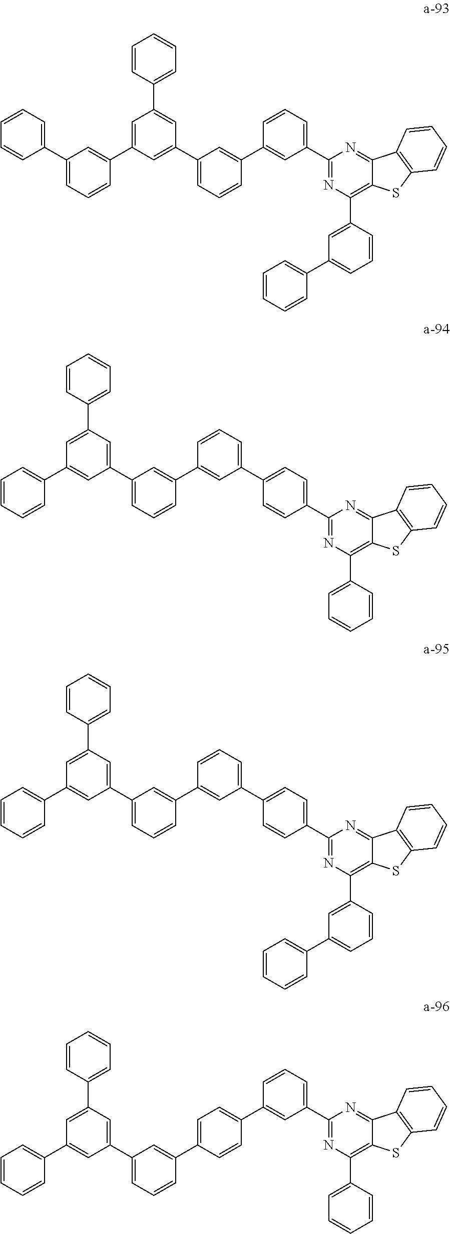 [SMILES: c1ccc(-c2cccc(-c3cc(-c4ccccc4)cc(-c4cccc(-c5cccc(-c6nc(-c7cccc(-c8ccccc8)c7)c7sc8ccccc8c7n6)c5)c4)c3)c2)cc1 ; c1ccc(-c2cc(-c3ccccc3)cc(-c3cccc(-c4cccc(-c5ccc(-c6nc(-c7ccccc7)c7sc8ccccc8c7n6)cc5)c4)c3)c2)cc1 ; c1ccc(-c2cc(-c3ccccc3)cc(-c3cccc(-c4cccc(-c5ccc(-c6nc(-c7cccc(-c8ccccc8)c7)c7sc8ccccc8c7n6)cc5)c4)c3)c2)cc1 ; c1ccc(-c2cc(-c3ccccc3)cc(-c3cccc(-c4ccc(-c5cccc(-c6nc(-c7ccccc7)c7sc8ccccc8c7n6)c5)cc4)c3)c2)cc1]

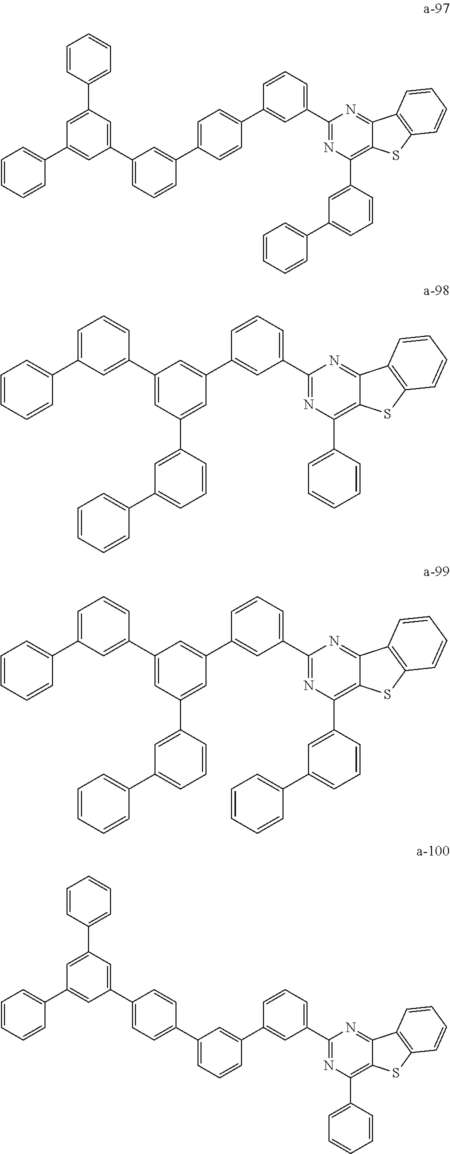 [SMILES: c1ccc(-c2cc(-c3ccccc3)cc(-c3cccc(-c4ccc(-c5cccc(-c6nc(-c7cccc(-c8ccccc8)c7)c7sc8ccccc8c7n6)c5)cc4)c3)c2)cc1 ; c1ccc(-c2cccc(-c3cc(-c4cccc(-c5ccccc5)c4)cc(-c4cccc(-c5nc(-c6ccccc6)c6sc7ccccc7c6n5)c4)c3)c2)cc1 ; c1ccc(-c2cccc(-c3cc(-c4cccc(-c5ccccc5)c4)cc(-c4cccc(-c5nc(-c6cccc(-c7ccccc7)c6)c6sc7ccccc7c6n5)c4)c3)c2)cc1 ; c1ccc(-c2cc(-c3ccccc3)cc(-c3ccc(-c4cccc(-c5cccc(-c6nc(-c7ccccc7)c7sc8ccccc8c7n6)c5)c4)cc3)c2)cc1]

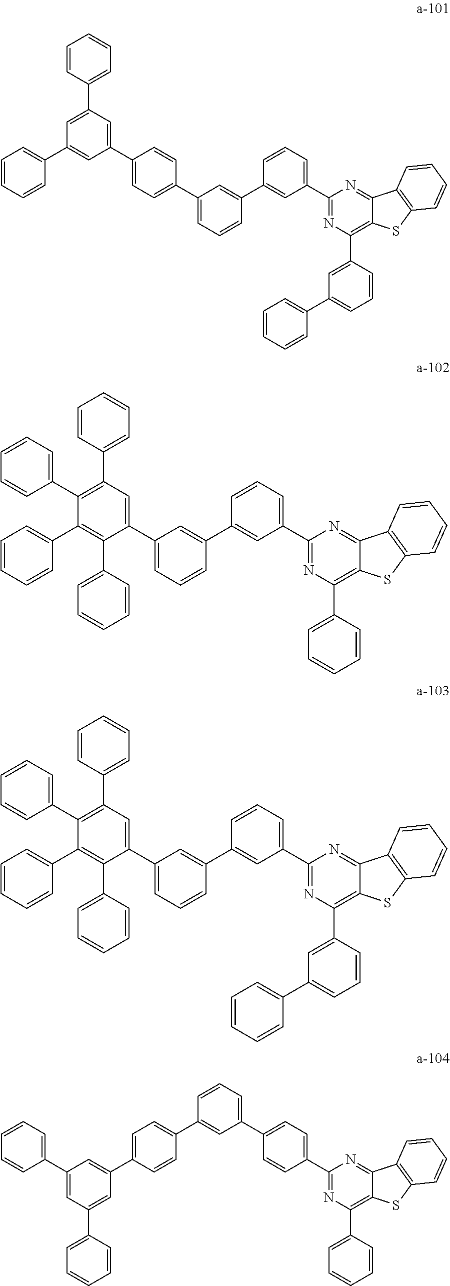 [SMILES: c1ccc(-c2cc(-c3ccccc3)cc(-c3ccc(-c4cccc(-c5cccc(-c6nc(-c7cccc(-c8ccccc8)c7)c7sc8ccccc8c7n6)c5)c4)cc3)c2)cc1 ; c1ccc(-c2cc(-c3cccc(-c4cccc(-c5nc(-c6ccccc6)c6sc7ccccc7c6n5)c4)c3)c(-c3ccccc3)c(-c3ccccc3)c2-c2ccccc2)cc1 ; c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4c3sc3ccccc34)c2)cc1 ; c1ccc(-c2cc(-c3ccccc3)cc(-c3ccc(-c4cccc(-c5ccc(-c6nc(-c7ccccc7)c7sc8ccccc8c7n6)cc5)c4)cc3)c2)cc1]

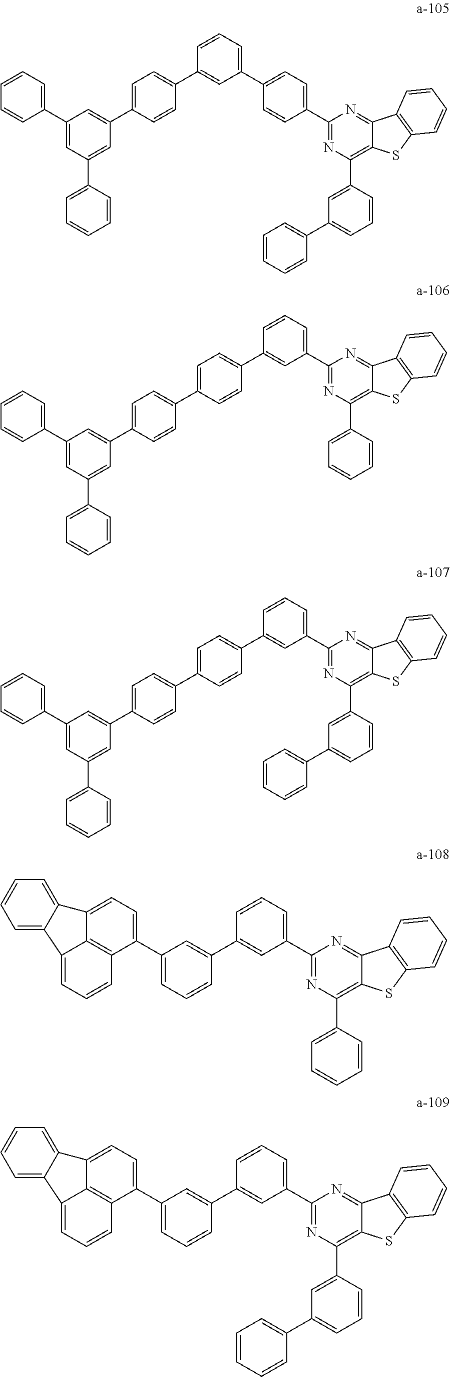 [SMILES: c1ccc(-c2cc(-c3ccccc3)cc(-c3ccc(-c4cccc(-c5ccc(-c6nc(-c7cccc(-c8ccccc8)c7)c7sc8ccccc8c7n6)cc5)c4)cc3)c2)cc1 ; c1ccc(-c2cc(-c3ccccc3)cc(-c3ccc(-c4ccc(-c5cccc(-c6nc(-c7ccccc7)c7sc8ccccc8c7n6)c5)cc4)cc3)c2)cc1 ; c1ccc(-c2cc(-c3ccccc3)cc(-c3ccc(-c4ccc(-c5cccc(-c6nc(-c7cccc(-c8ccccc8)c7)c7sc8ccccc8c7n6)c5)cc4)cc3)c2)cc1 ; c1ccc(-c2nc(-c3cccc(-c4cccc(-c5ccc6c7c(cccc57)-c5ccccc5-6)c4)c3)nc3c2sc2ccccc23)cc1 ; c1ccc(-c2cccc(-c3nc(-c4cccc(-c5cccc(-c6ccc7c8c(cccc68)-c6ccccc6-7)c5)c4)nc4c3sc3ccccc34)c2)cc1]

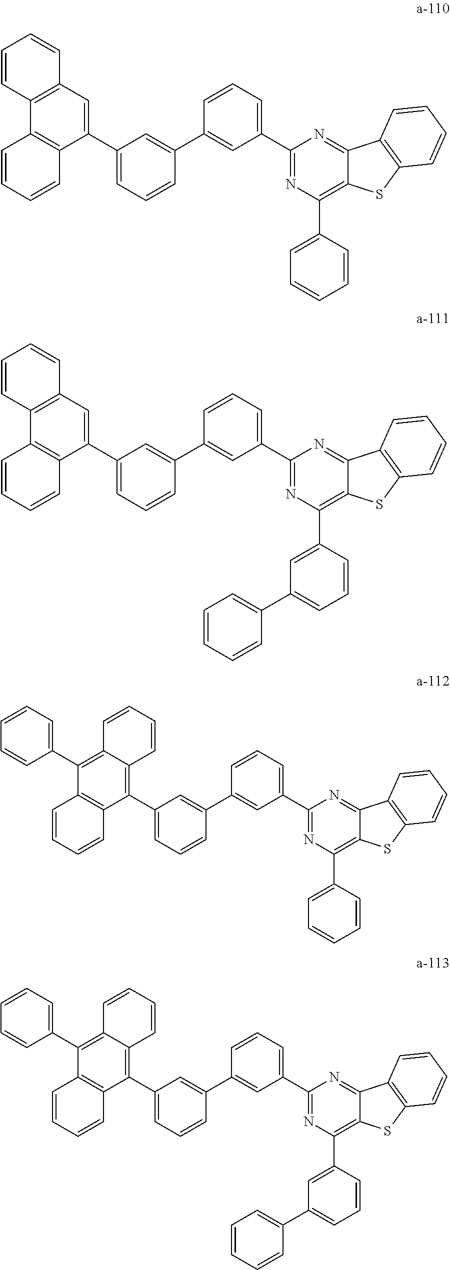 [SMILES: c1ccc(-c2nc(-c3cccc(-c4cccc(-c5cc6ccccc6c6ccccc56)c4)c3)nc3c2sc2ccccc23)cc1 ; c1ccc(-c2cccc(-c3nc(-c4cccc(-c5cccc(-c6cc7ccccc7c7ccccc67)c5)c4)nc4c3sc3ccccc34)c2)cc1 ; c1ccc(-c2c3ccccc3c(-c3cccc(-c4cccc(-c5nc(-c6ccccc6)c6sc7ccccc7c6n5)c4)c3)c3ccccc23)cc1 ; c1ccc(-c2cccc(-c3nc(-c4cccc(-c5cccc(-c6c7ccccc7c(-c7ccccc7)c7ccccc67)c5)c4)nc4c3sc3ccccc34)c2)cc1]

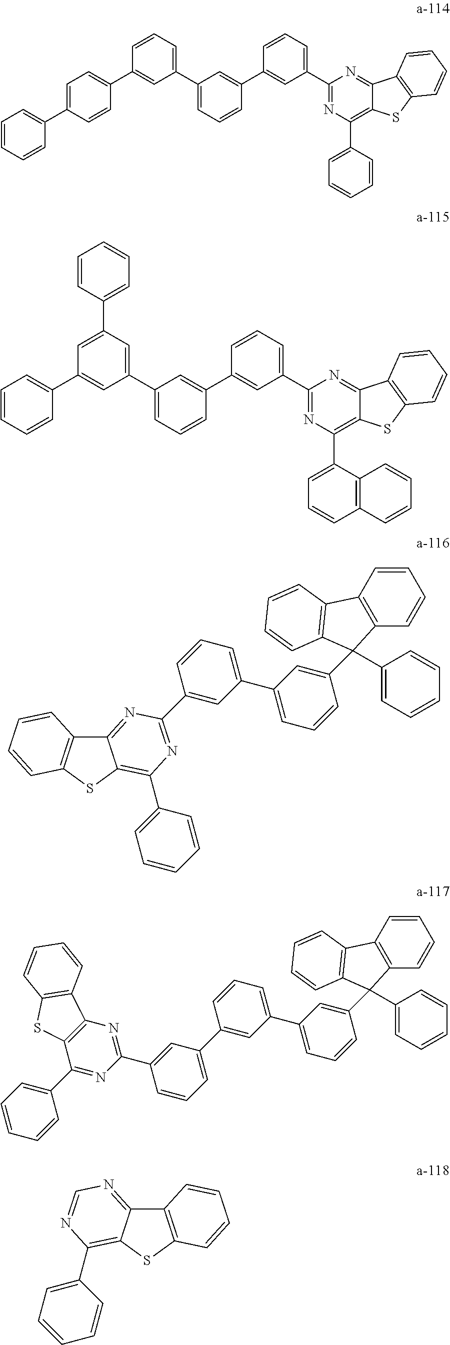 [SMILES: c1ccc(-c2ccc(-c3cccc(-c4cccc(-c5cccc(-c6nc(-c7ccccc7)c7sc8ccccc8c7n6)c5)c4)c3)cc2)cc1 ; c1ccc(-c2cc(-c3ccccc3)cc(-c3cccc(-c4cccc(-c5nc(-c6cccc7ccccc67)c6sc7ccccc7c6n5)c4)c3)c2)cc1 ; c1ccc(-c2nc(-c3cccc(-c4cccc(C5(c6ccccc6)c6ccccc6-c6ccccc65)c4)c3)nc3c2sc2ccccc23)cc1 ; c1ccc(-c2nc(-c3cccc(-c4cccc(-c5cccc(C6(c7ccccc7)c7ccccc7-c7ccccc76)c5)c4)c3)nc3c2sc2ccccc23)cc1 ; c1ccc(-c2ncnc3c2sc2ccccc23)cc1]

Group of X₁=O in Formula 1-1

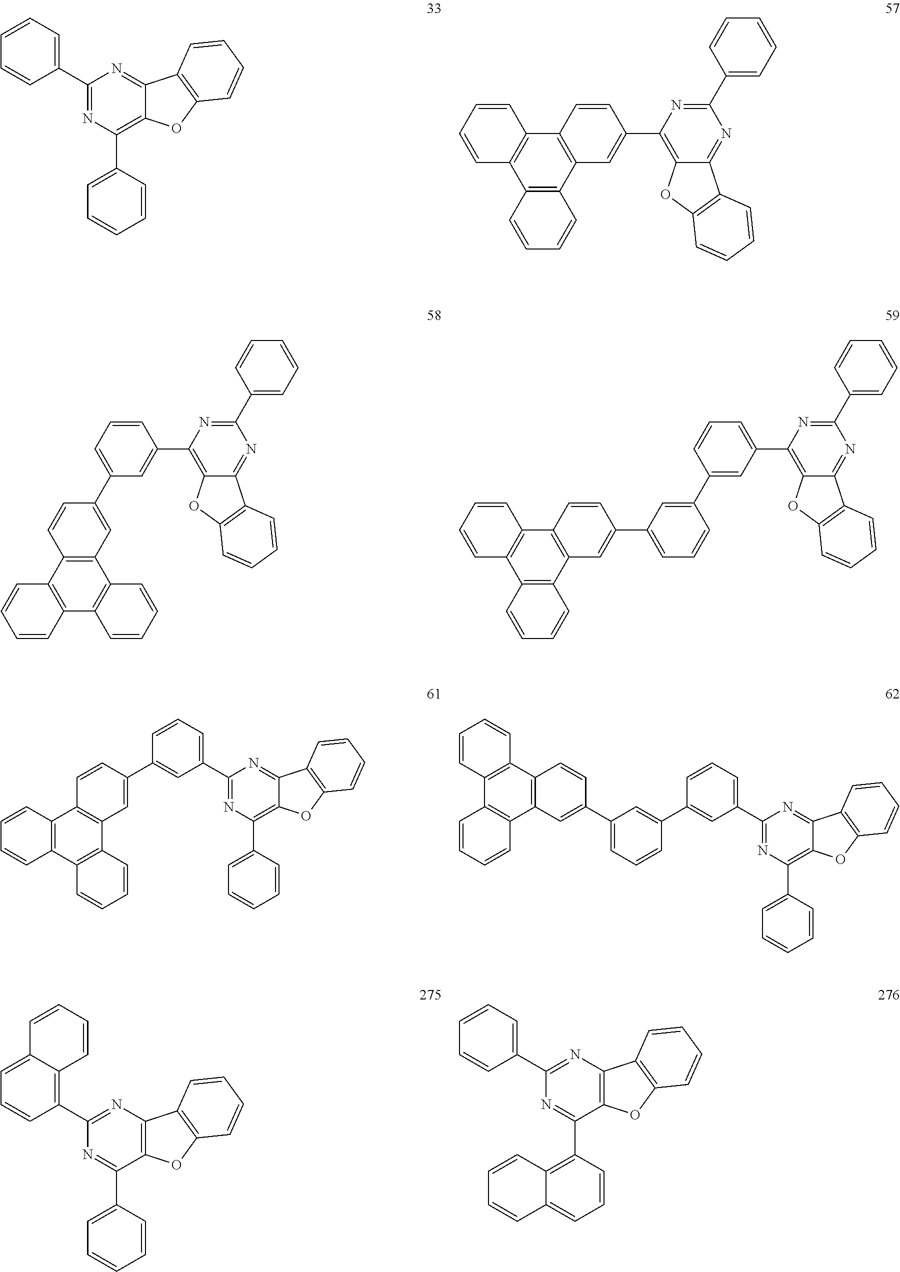

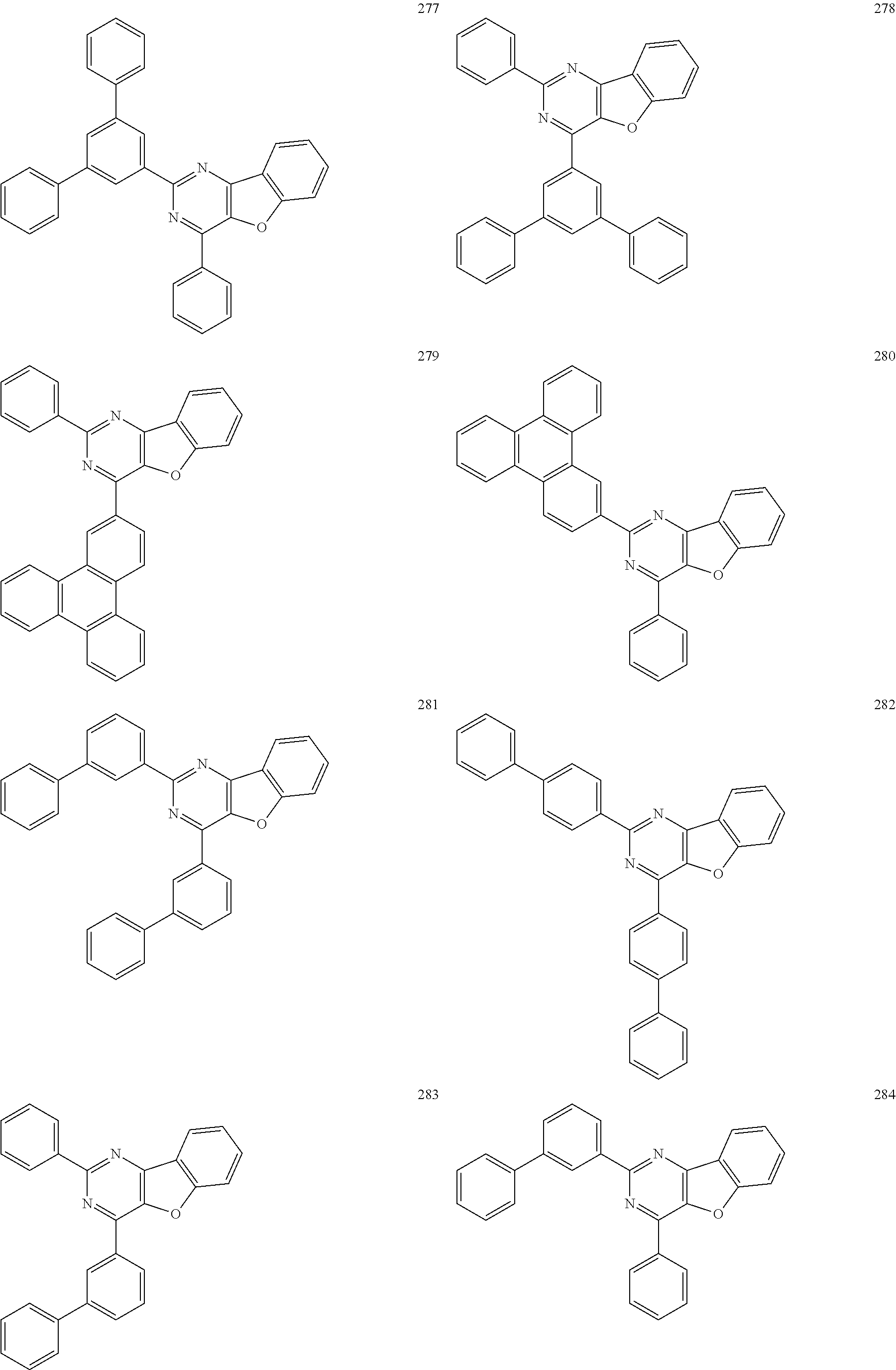

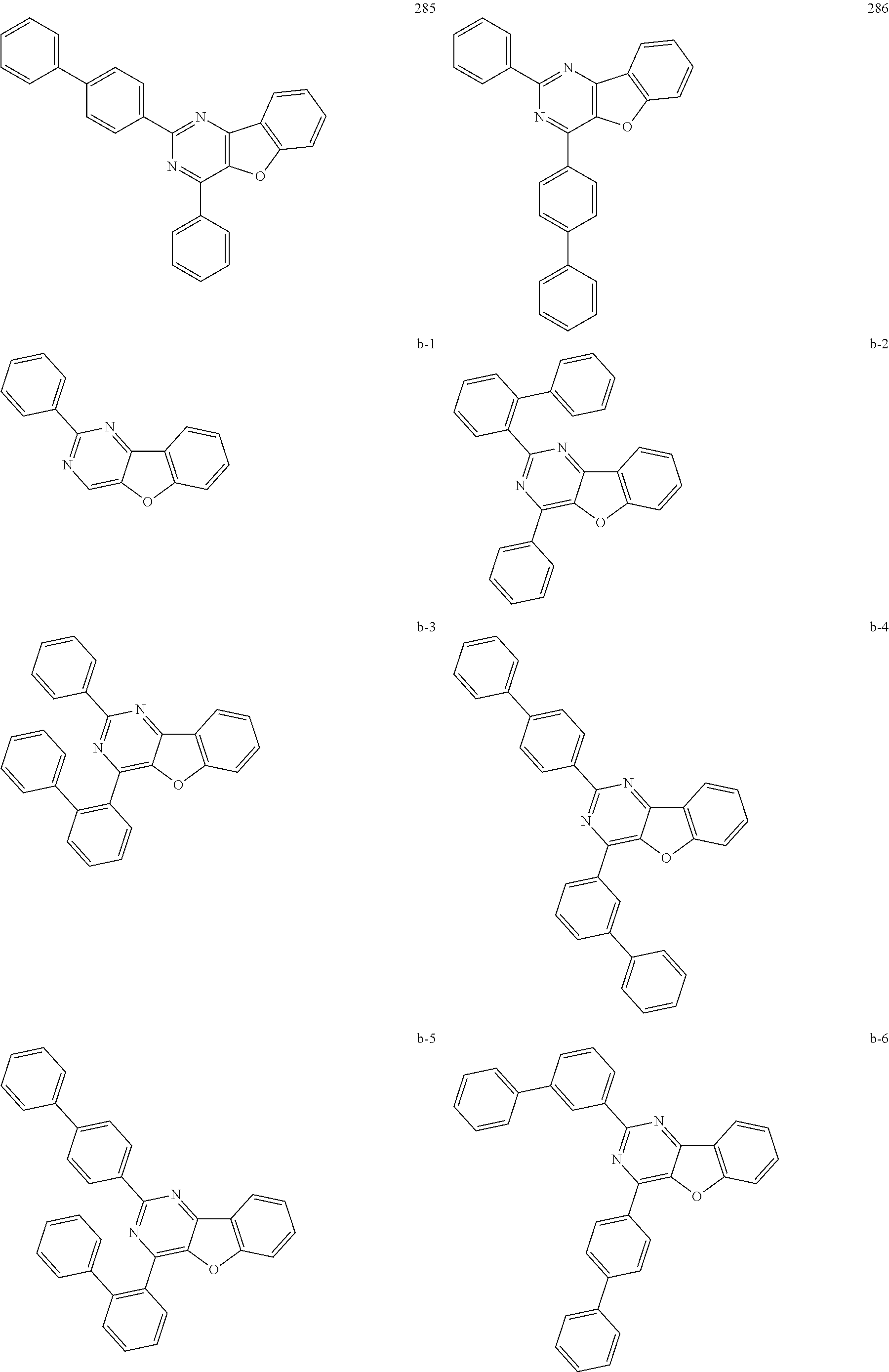

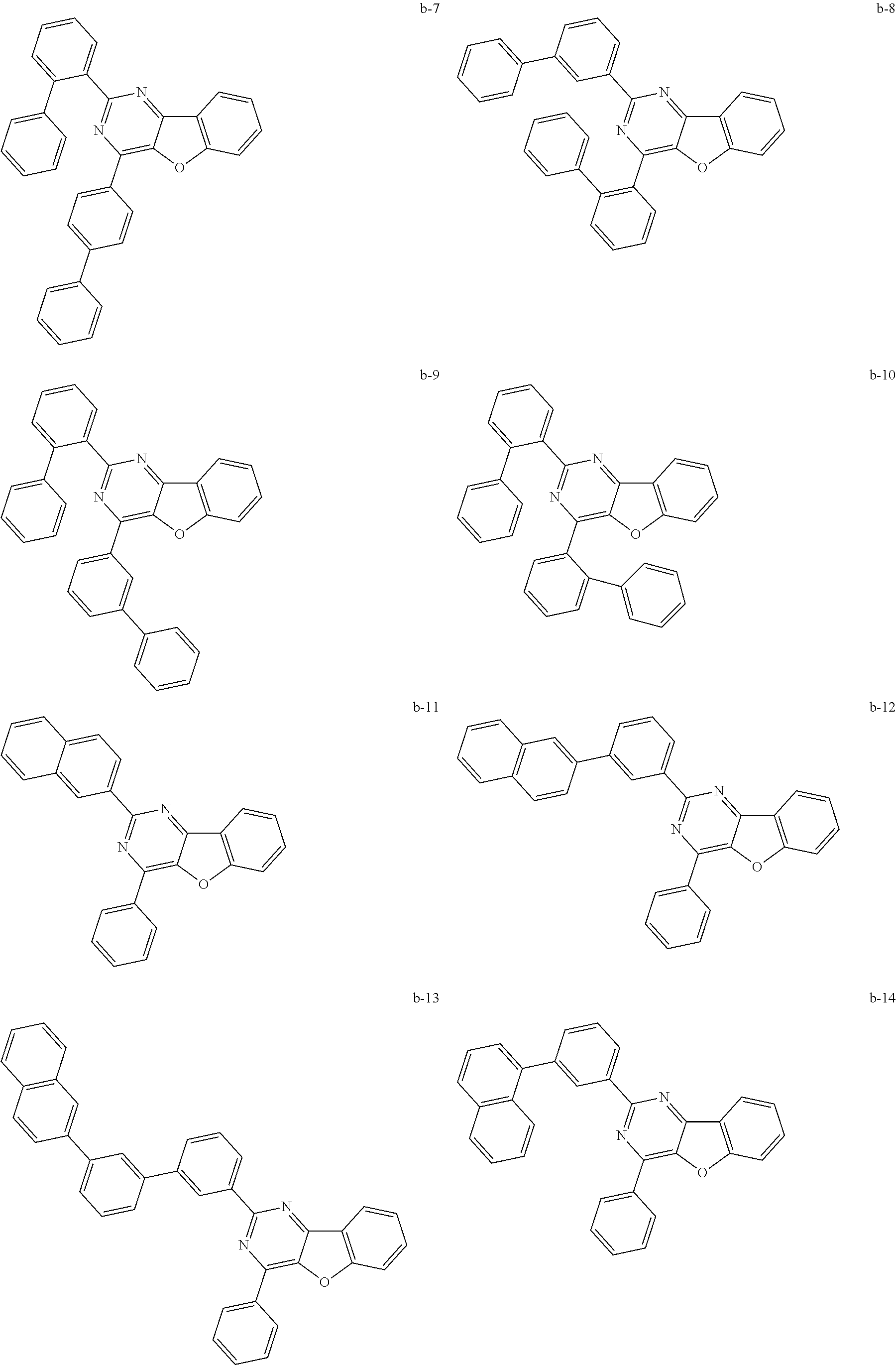

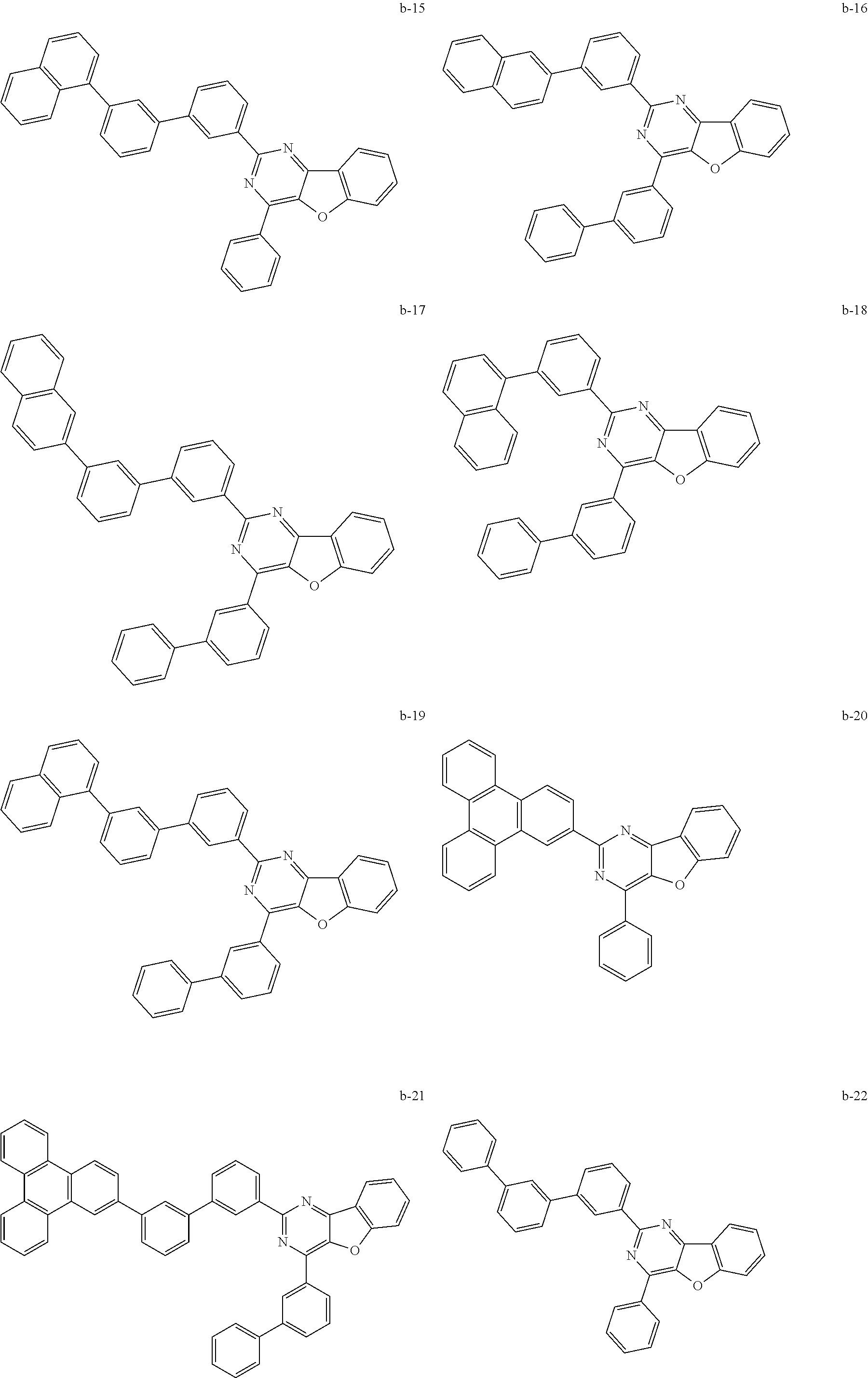

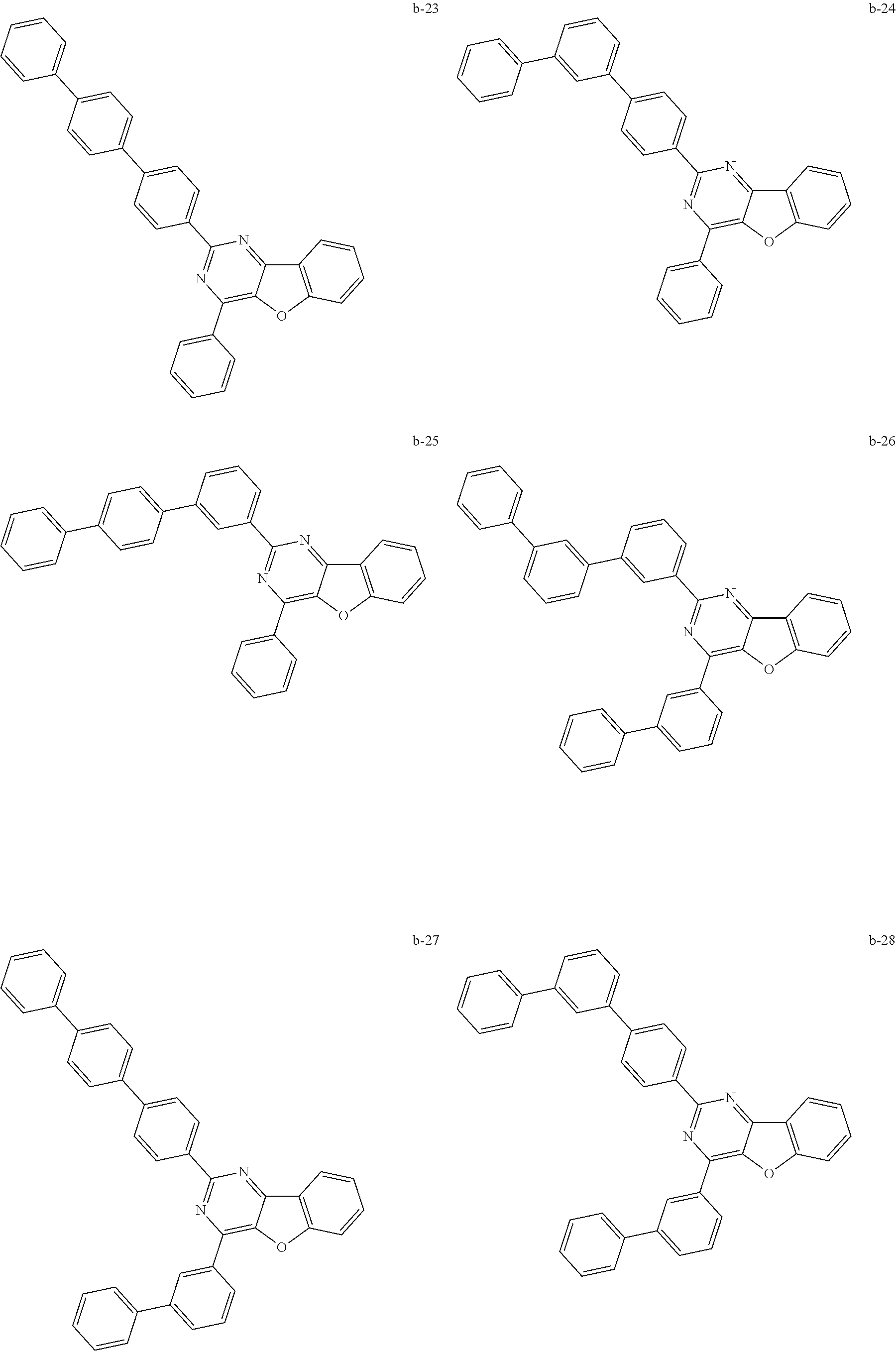 [SMILES: c1ccc(-c2ccc(-c3ccc(-c4nc(-c5ccccc5)c5oc6ccccc6c5n4)cc3)cc2)cc1 ; c1ccc(-c2cccc(-c3ccc(-c4nc(-c5ccccc5)c5oc6ccccc6c5n4)cc3)c2)cc1 ; c1ccc(-c2ccc(-c3cccc(-c4nc(-c5ccccc5)c5oc6ccccc6c5n4)c3)cc2)cc1 ; c1ccc(-c2cccc(-c3cccc(-c4nc(-c5cccc(-c6ccccc6)c5)c5oc6ccccc6c5n4)c3)c2)cc1 ; c1ccc(-c2ccc(-c3ccc(-c4nc(-c5cccc(-c6ccccc6)c5)c5oc6ccccc6c5n4)cc3)cc2)cc1 ; c1ccc(-c2cccc(-c3ccc(-c4nc(-c5cccc(-c6ccccc6)c5)c5oc6ccccc6c5n4)cc3)c2)cc1]

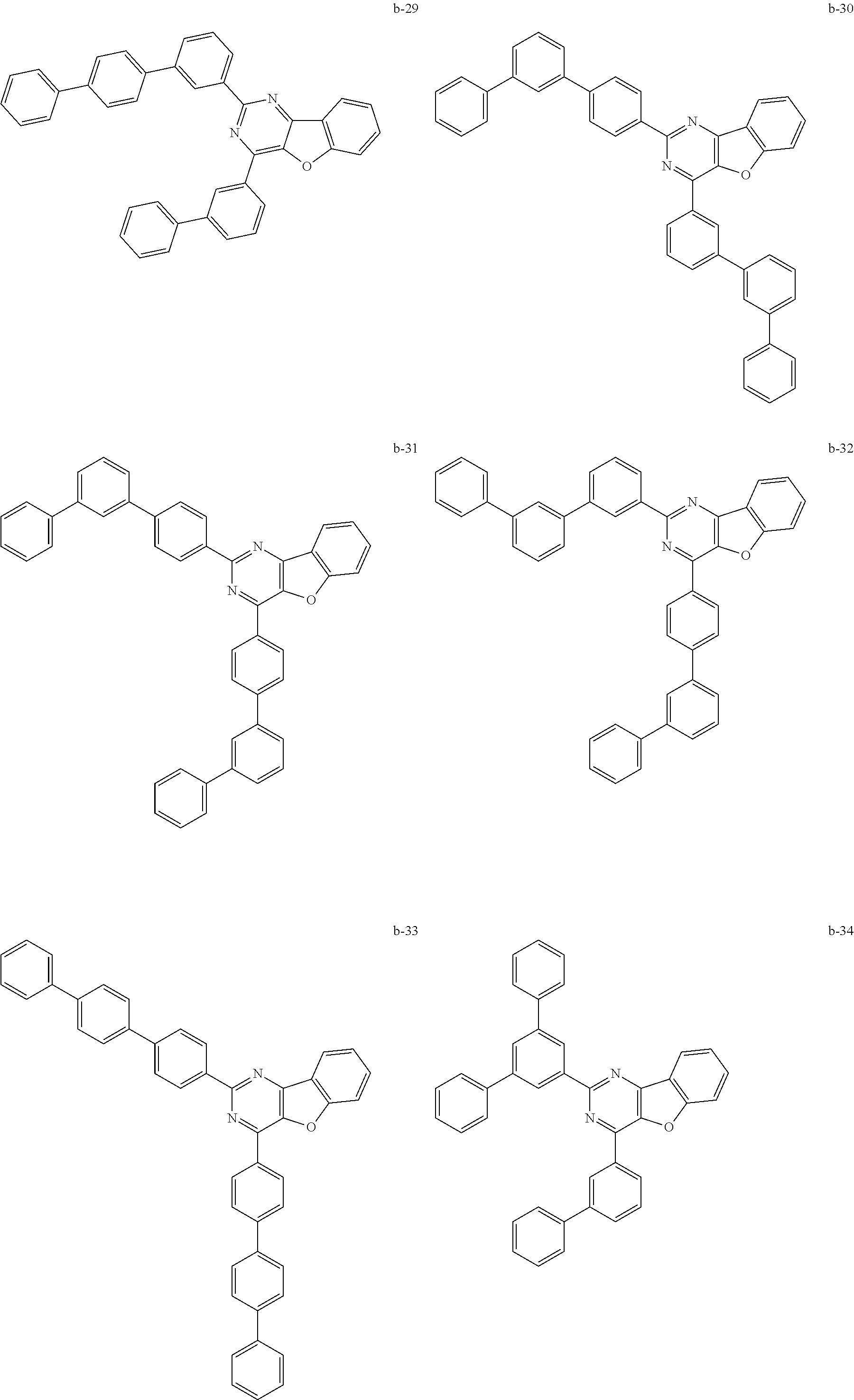

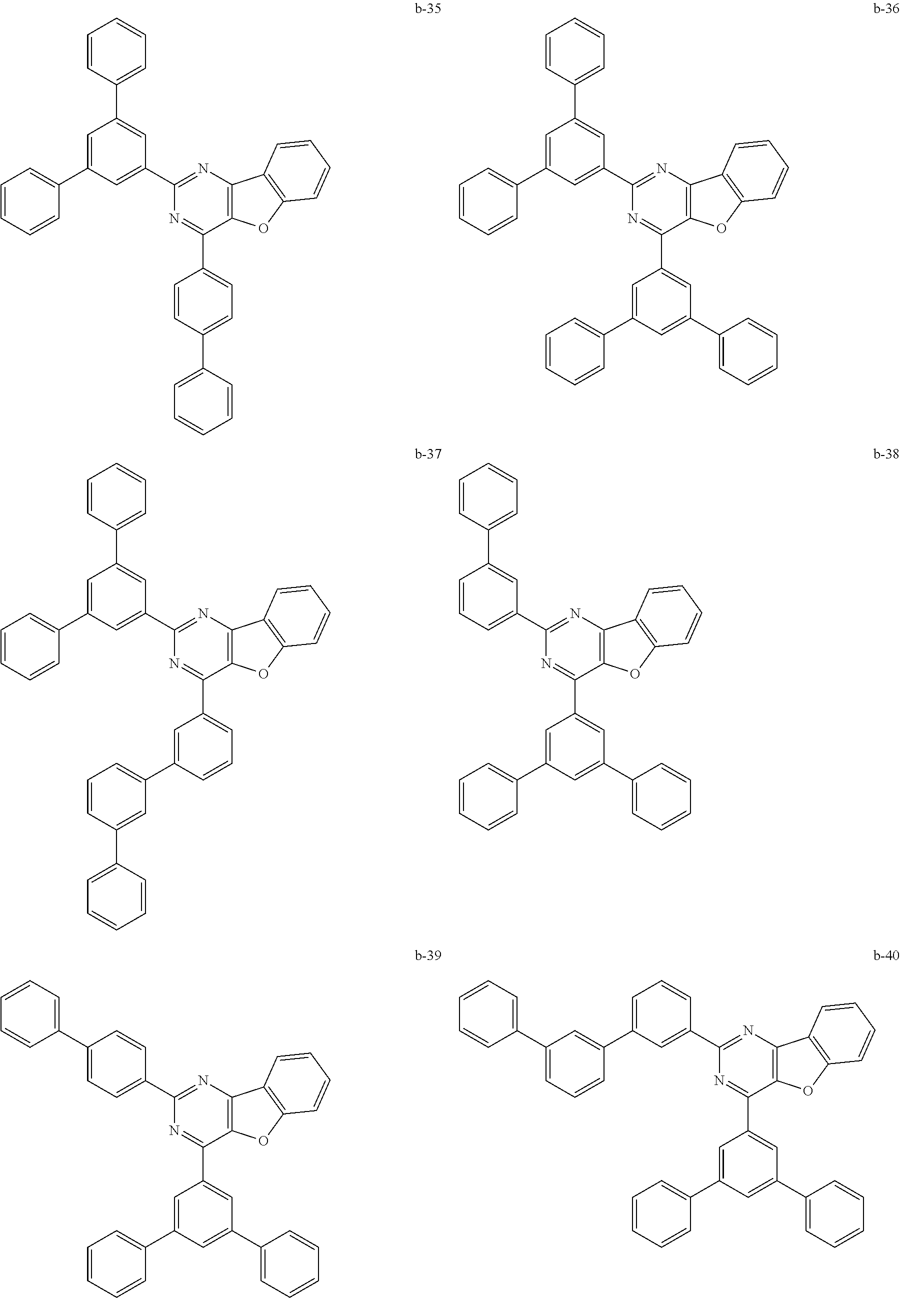 [SMILES: c1ccc(-c2ccc(-c3nc(-c4cc(-c5ccccc5)cc(-c5ccccc5)c4)nc4c3oc3ccccc34)cc2)cc1 ; c1ccc(-c2cc(-c3ccccc3)cc(-c3nc(-c4cc(-c5ccccc5)cc(-c5ccccc5)c4)c4oc5ccccc5c4n3)c2)cc1 ; c1ccc(-c2cccc(-c3cccc(-c4nc(-c5cc(-c6ccccc6)cc(-c6ccccc6)c5)nc5c4oc4ccccc45)c3)c2)cc1 ; c1ccc(-c2cccc(-c3nc(-c4cc(-c5ccccc5)cc(-c5ccccc5)c4)c4oc5ccccc5c4n3)c2)cc1 ; c1ccc(-c2ccc(-c3nc(-c4cc(-c5ccccc5)cc(-c5ccccc5)c4)c4oc5ccccc5c4n3)cc2)cc1 ; c1ccc(-c2cccc(-c3cccc(-c4nc(-c5cc(-c6ccccc6)cc(-c6ccccc6)c5)c5oc6ccccc6c5n4)c3)c2)cc1]

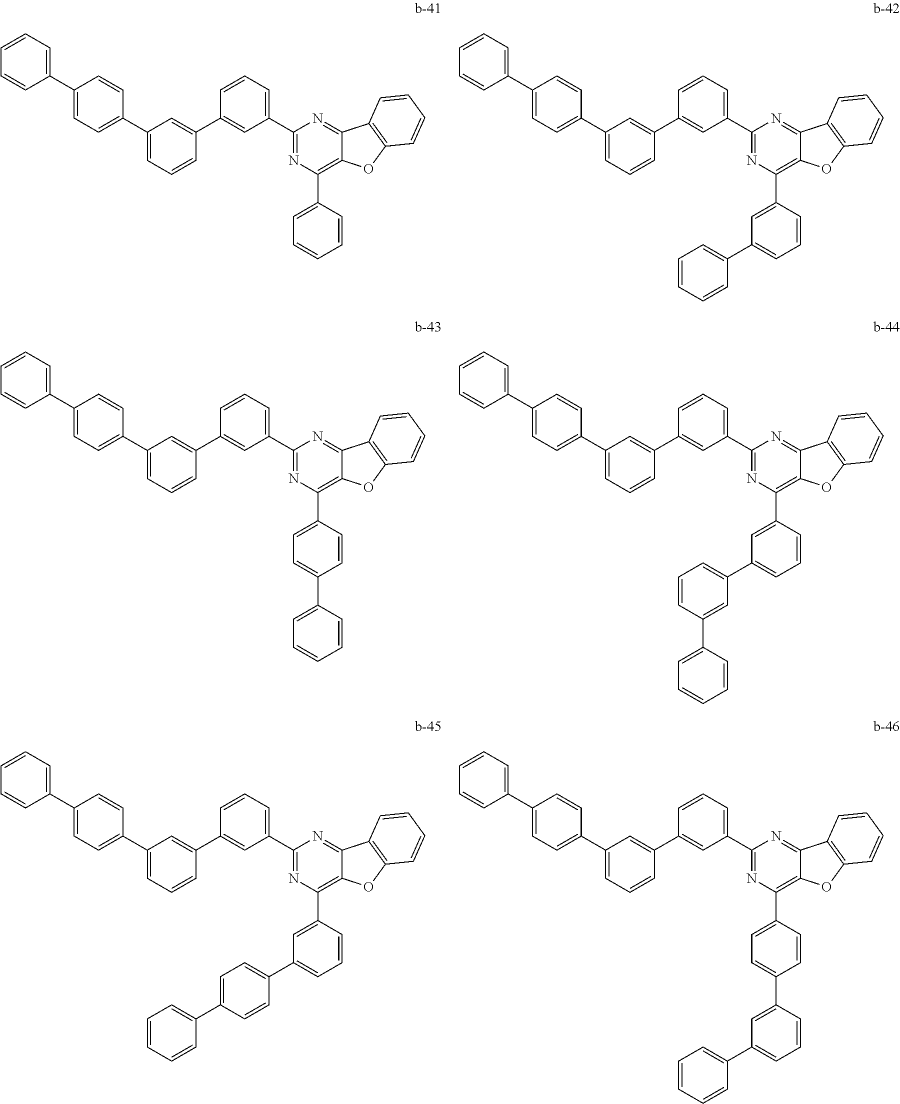

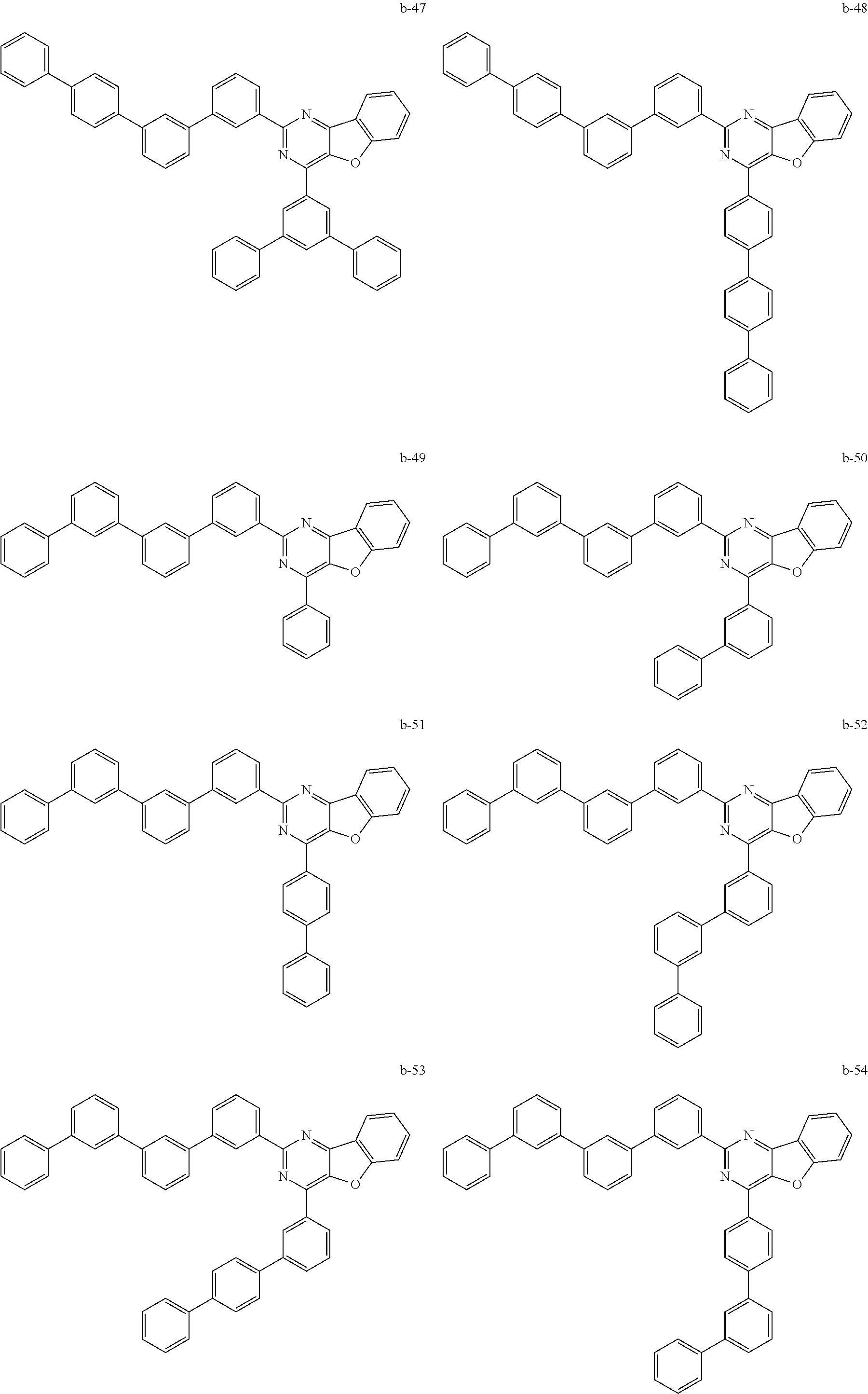

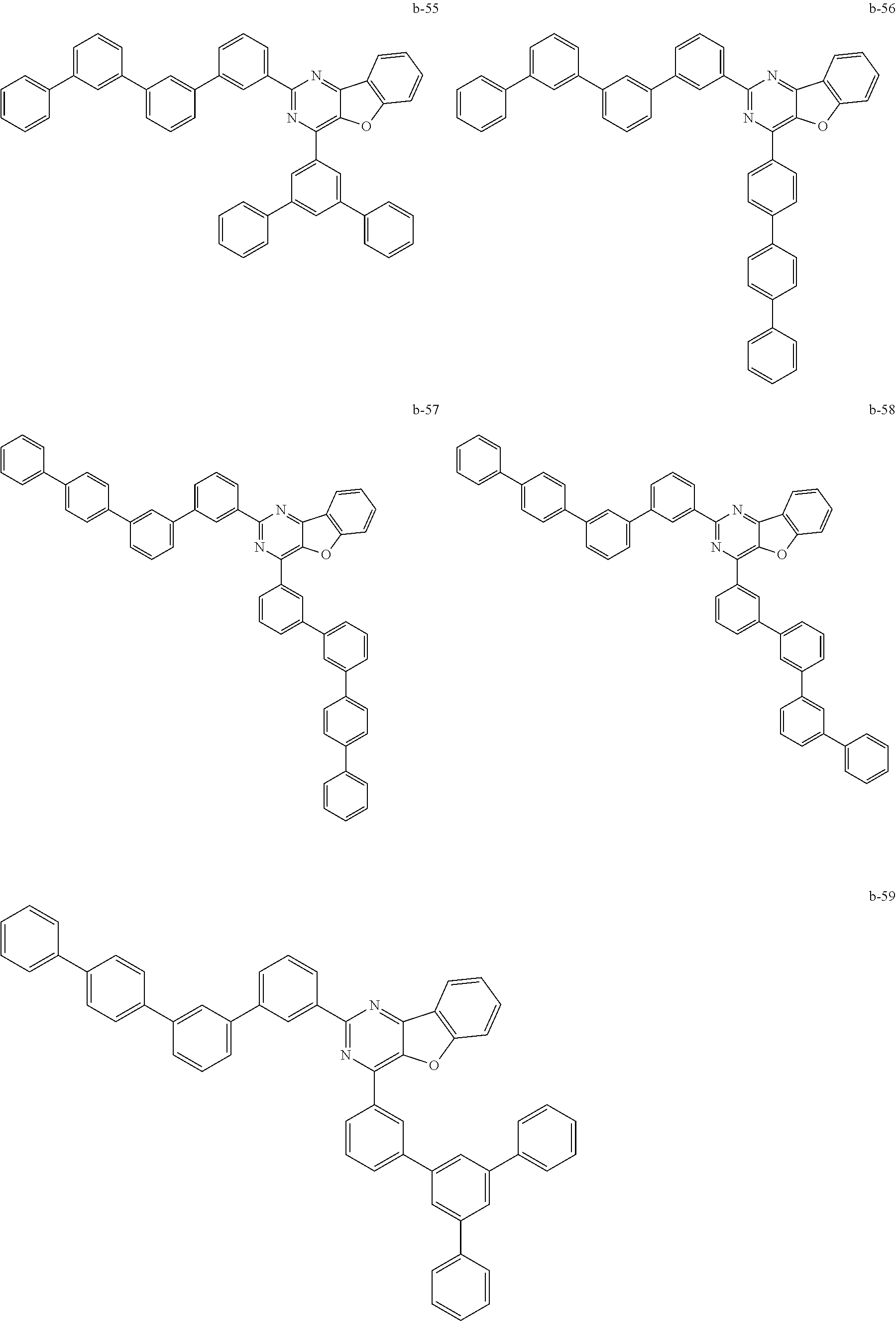

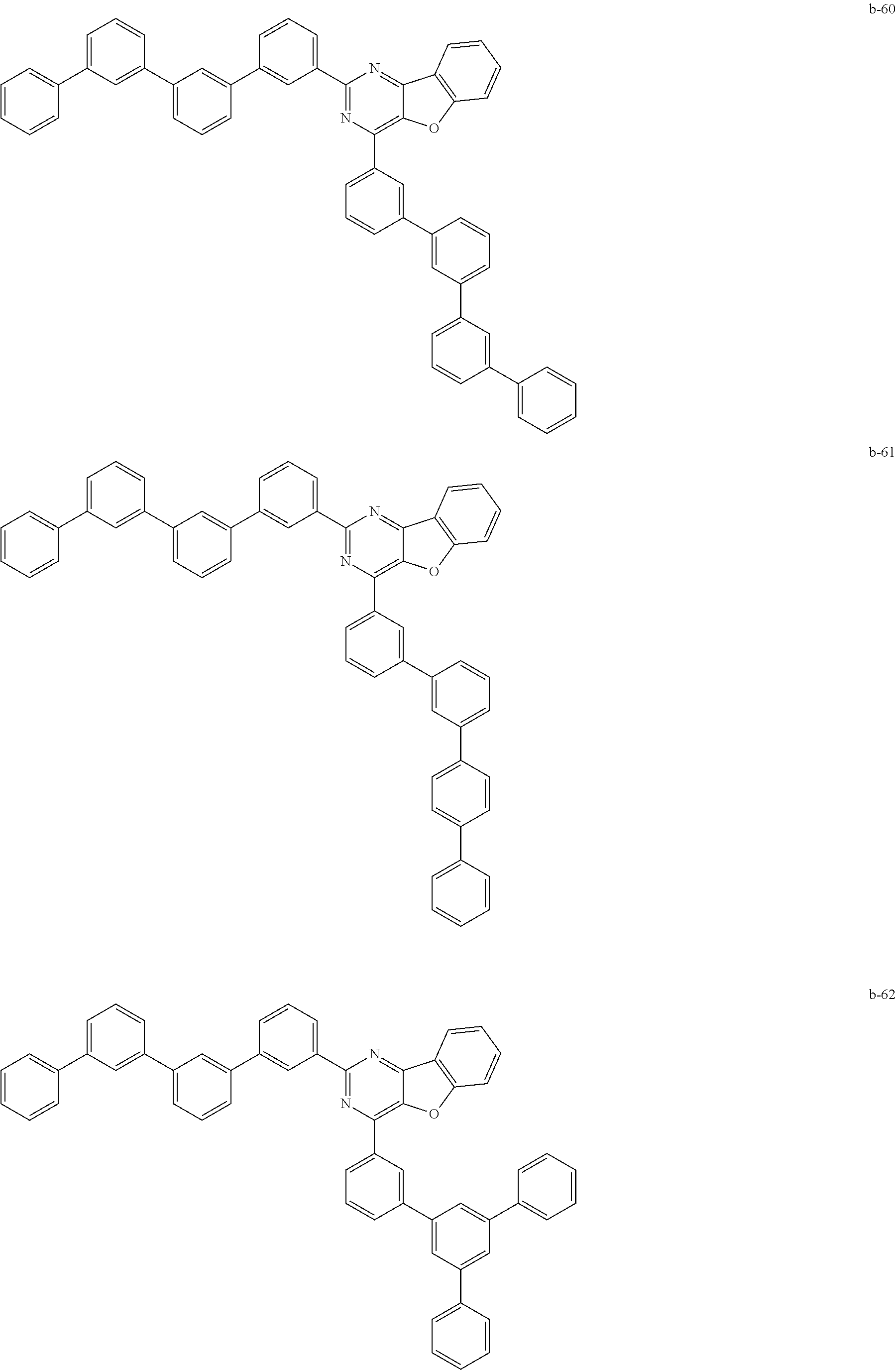 [SMILES: c1ccc(-c2cccc(-c3cccc(-c4cccc(-c5nc(-c6cccc(-c7cccc(-c8cccc(-c9ccccc9)c8)c7)c6)c6oc7ccccc7c6n5)c4)c3)c2)cc1 ; c1ccc(-c2ccc(-c3cccc(-c4cccc(-c5nc(-c6cccc(-c7cccc(-c8cccc(-c9ccccc9)c8)c7)c6)nc6c5oc5ccccc56)c4)c3)cc2)cc1 ; c1ccc(-c2cccc(-c3cccc(-c4cccc(-c5nc(-c6cccc(-c7cc(-c8ccccc8)cc(-c8ccccc8)c7)c6)c6oc7ccccc7c6n5)c4)c3)c2)cc1]

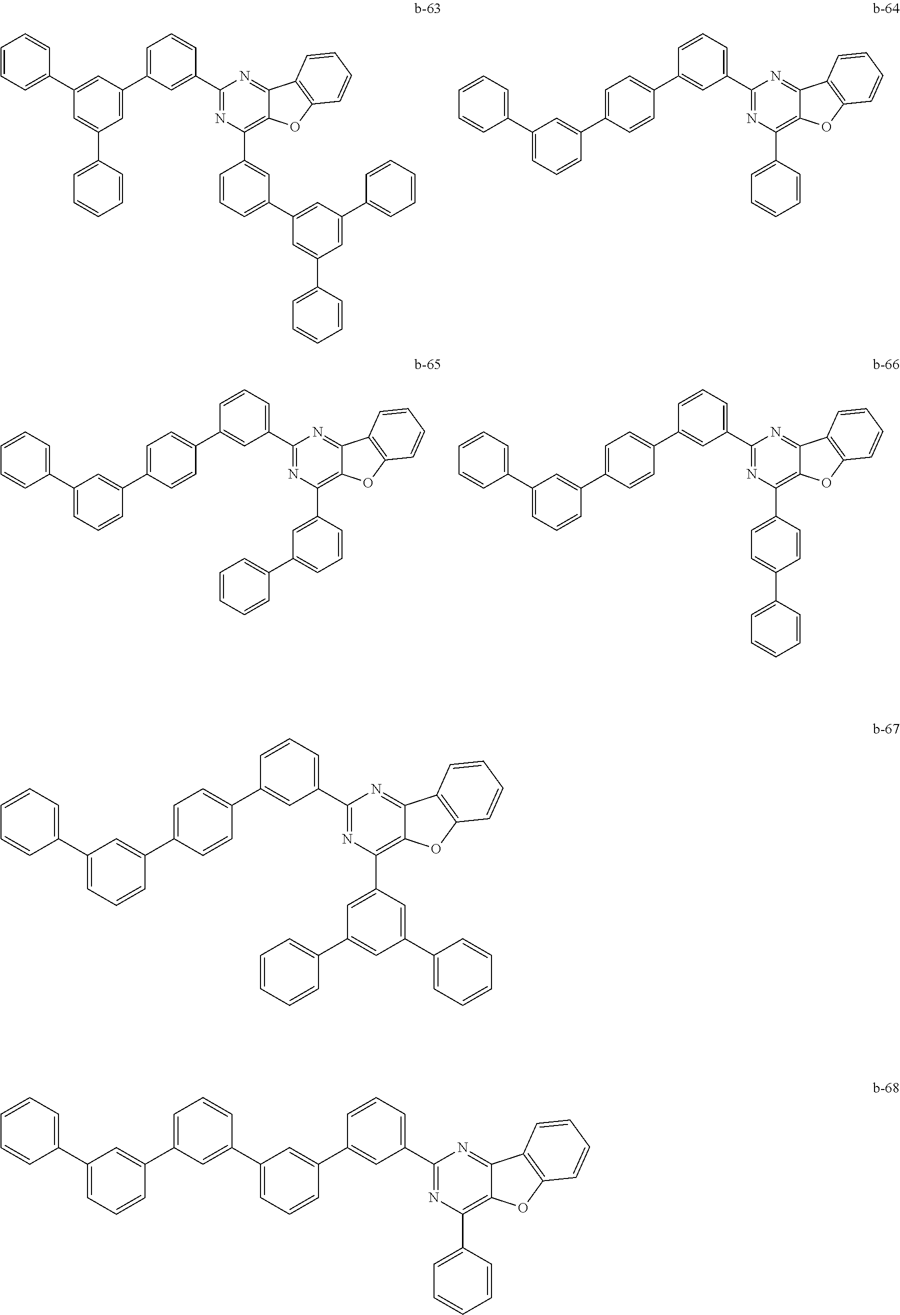

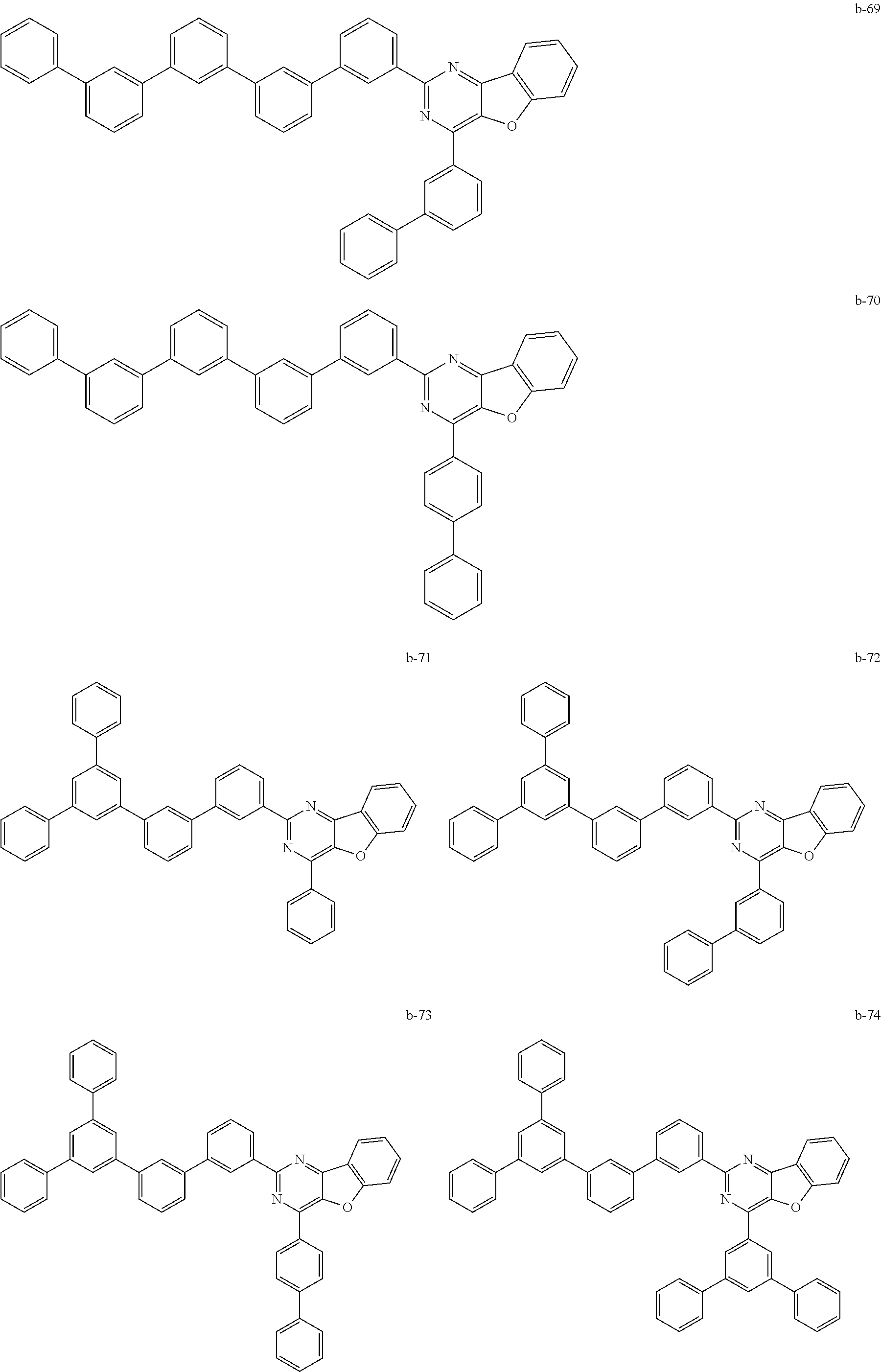

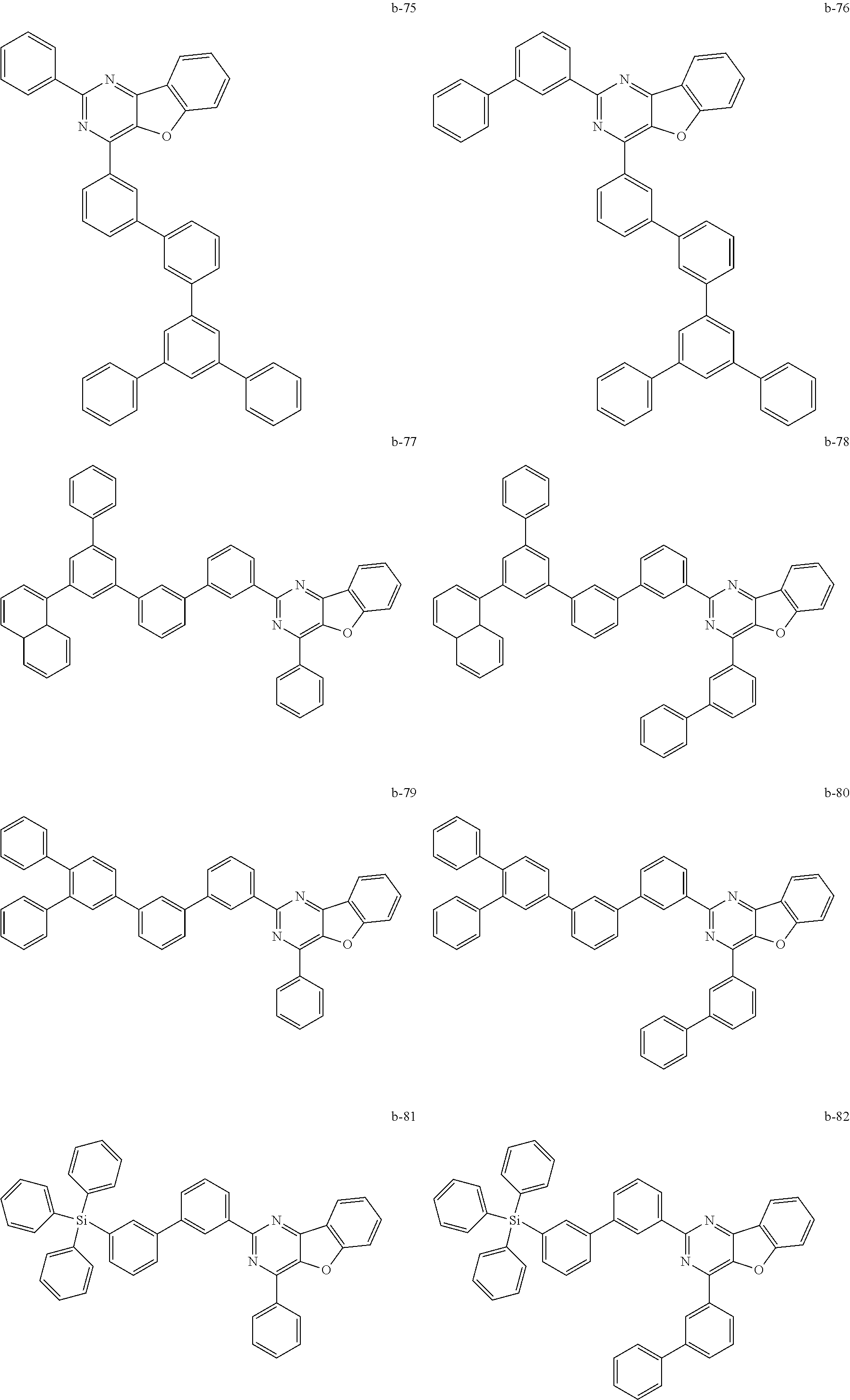

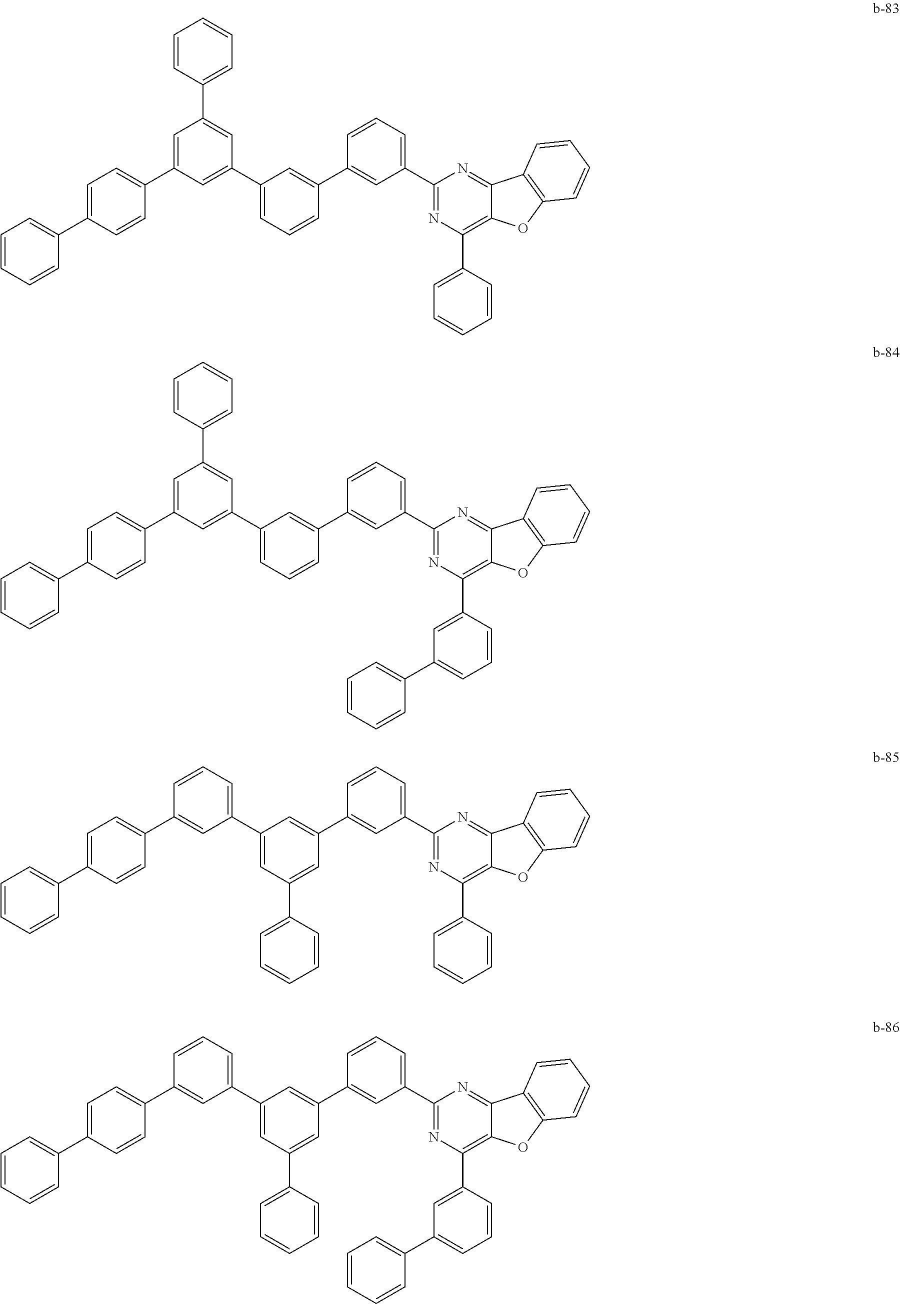

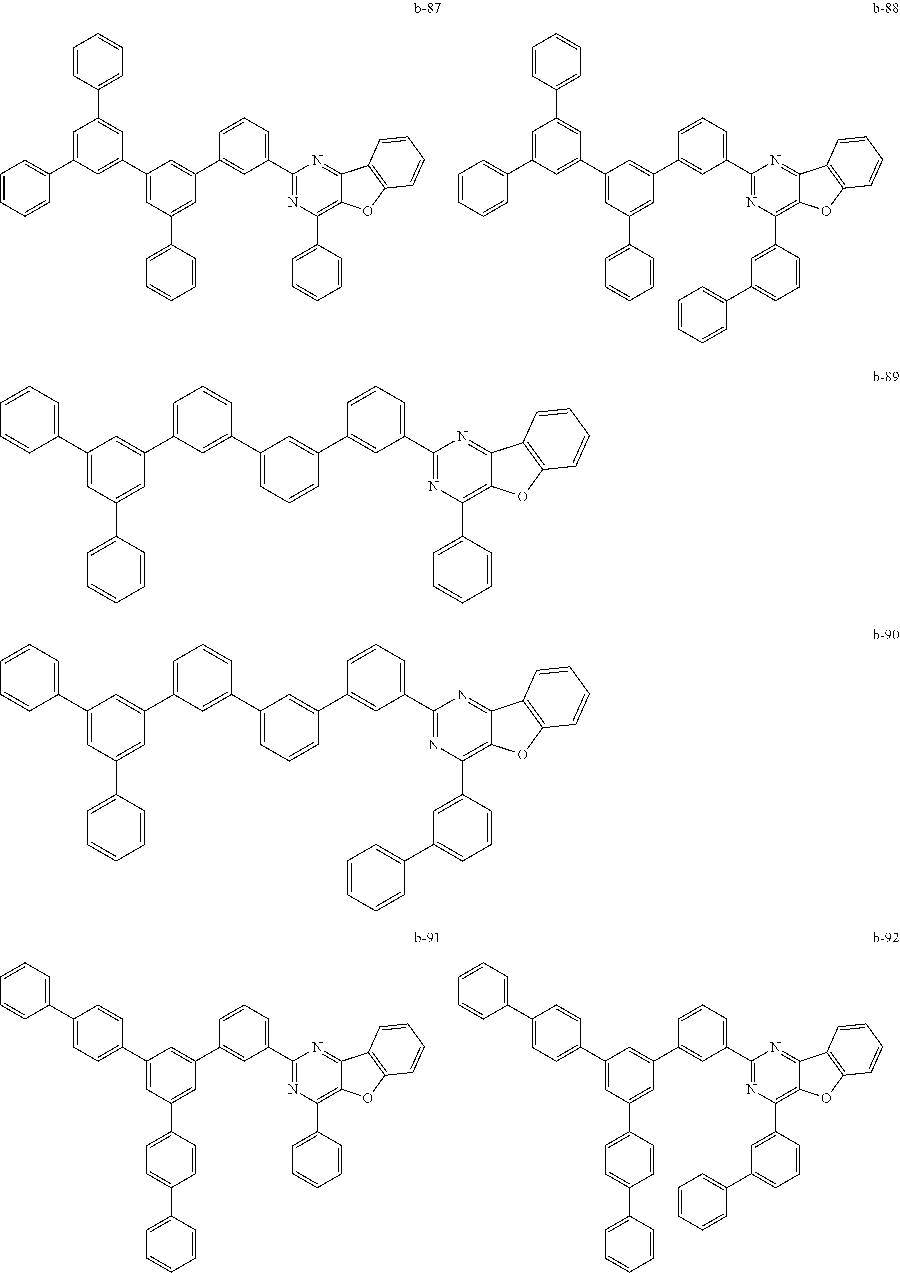

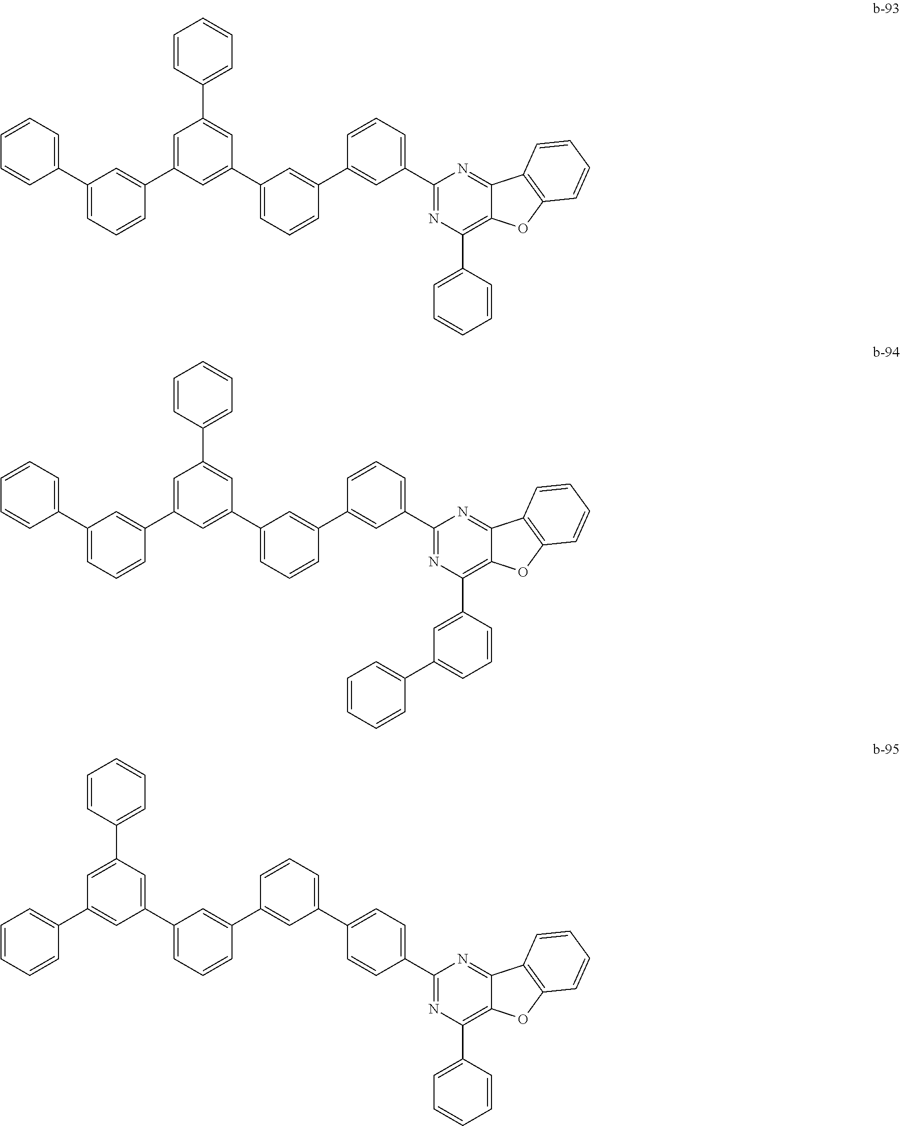 [SMILES: c1ccc(-c2cccc(-c3cc(-c4ccccc4)cc(-c4cccc(-c5cccc(-c6nc(-c7ccccc7)c7oc8ccccc8c7n6)c5)c4)c3)c2)cc1 ; c1ccc(-c2cccc(-c3cc(-c4ccccc4)cc(-c4cccc(-c5cccc(-c6nc(-c7cccc(-c8ccccc8)c7)c7oc8ccccc8c7n6)c5)c4)c3)c2)cc1 ; c1ccc(-c2cc(-c3ccccc3)cc(-c3cccc(-c4cccc(-c5ccc(-c6nc(-c7ccccc7)c7oc8ccccc8c7n6)cc5)c4)c3)c2)cc1]

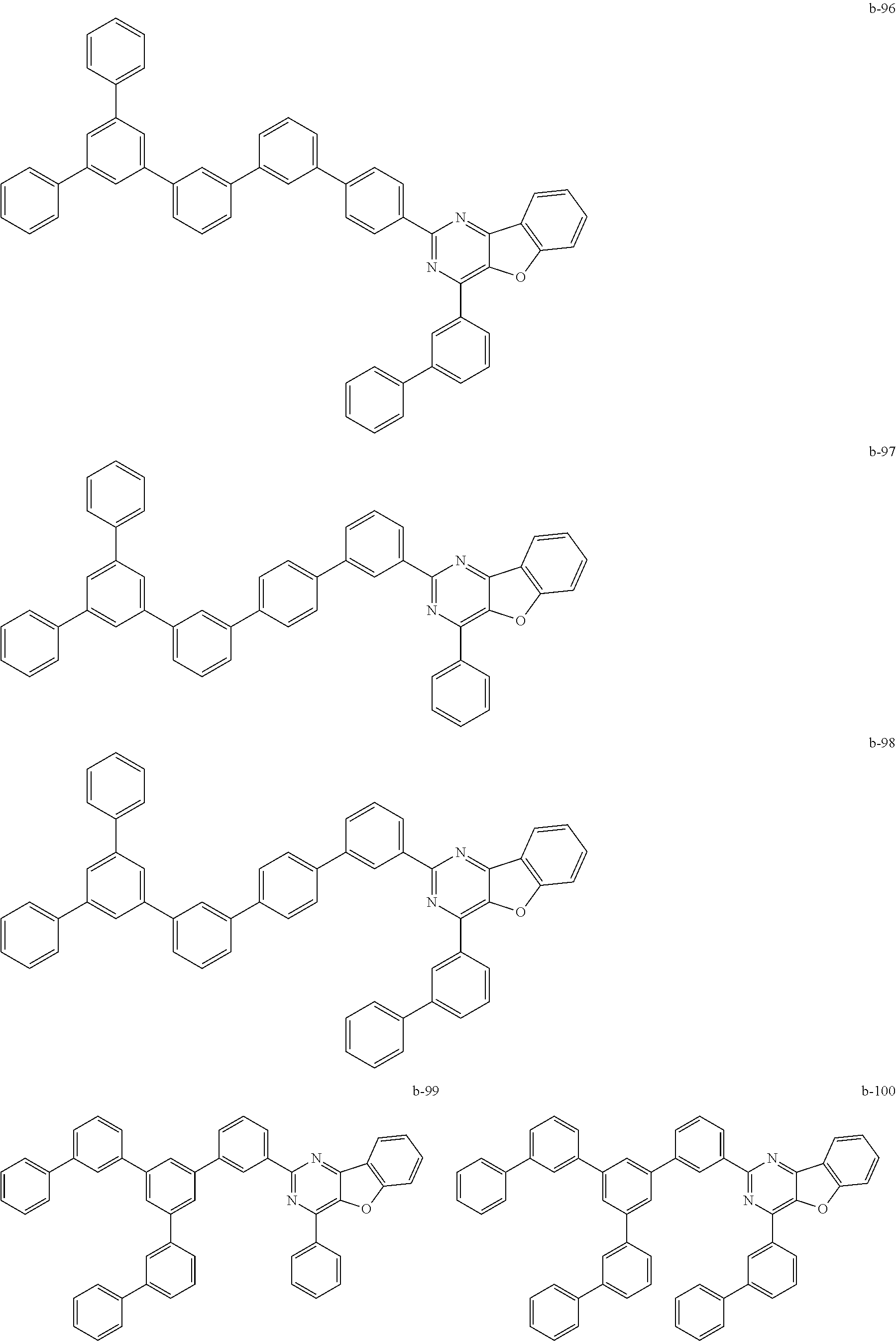

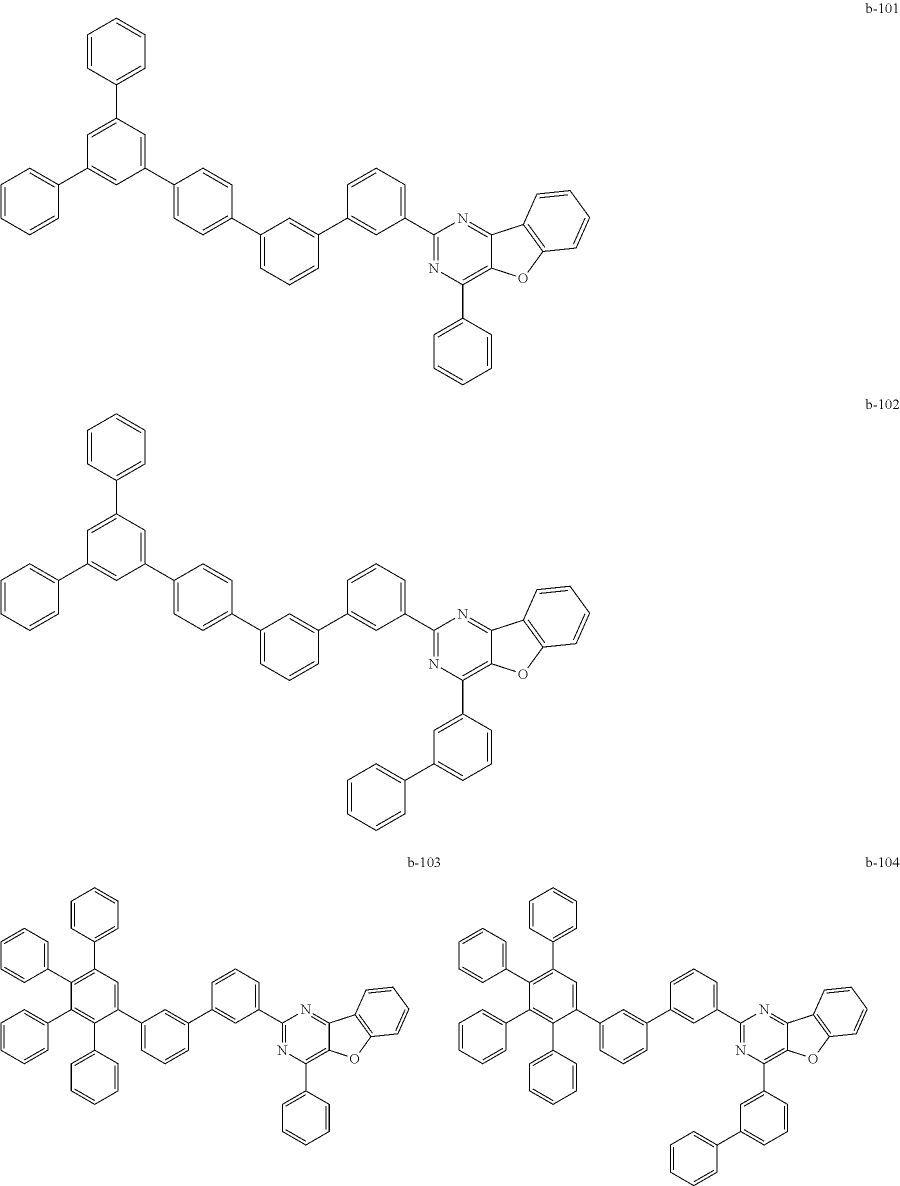 [SMILES: c1ccc(-c2cc(-c3ccccc3)cc(-c3ccc(-c4cccc(-c5cccc(-c6nc(-c7ccccc7)c7oc8ccccc8c7n6)c5)c4)cc3)c2)cc1 ; c1ccc(-c2cc(-c3ccccc3)cc(-c3ccc(-c4cccc(-c5cccc(-c6nc(-c7cccc(-c8ccccc8)c7)c7oc8ccccc8c7n6)c5)c4)cc3)c2)cc1 ; c1ccc(-c2cc(-c3cccc(-c4cccc(-c5nc(-c6ccccc6)c6oc7ccccc7c6n5)c4)c3)c(-c3ccccc3)c(-c3ccccc3)c2-c2ccccc2)cc1 ; c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4c3oc3ccccc34)c2)cc1]

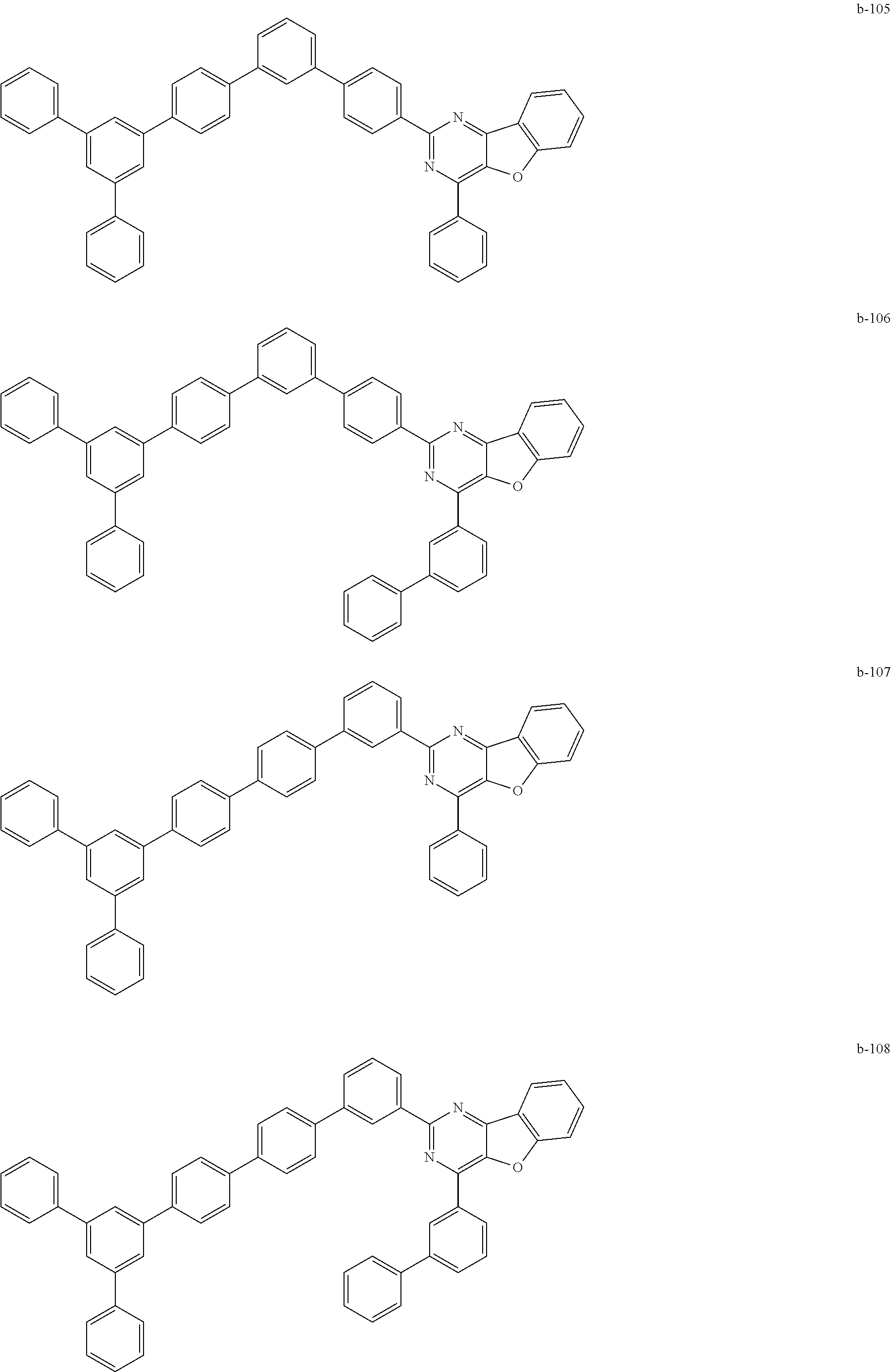

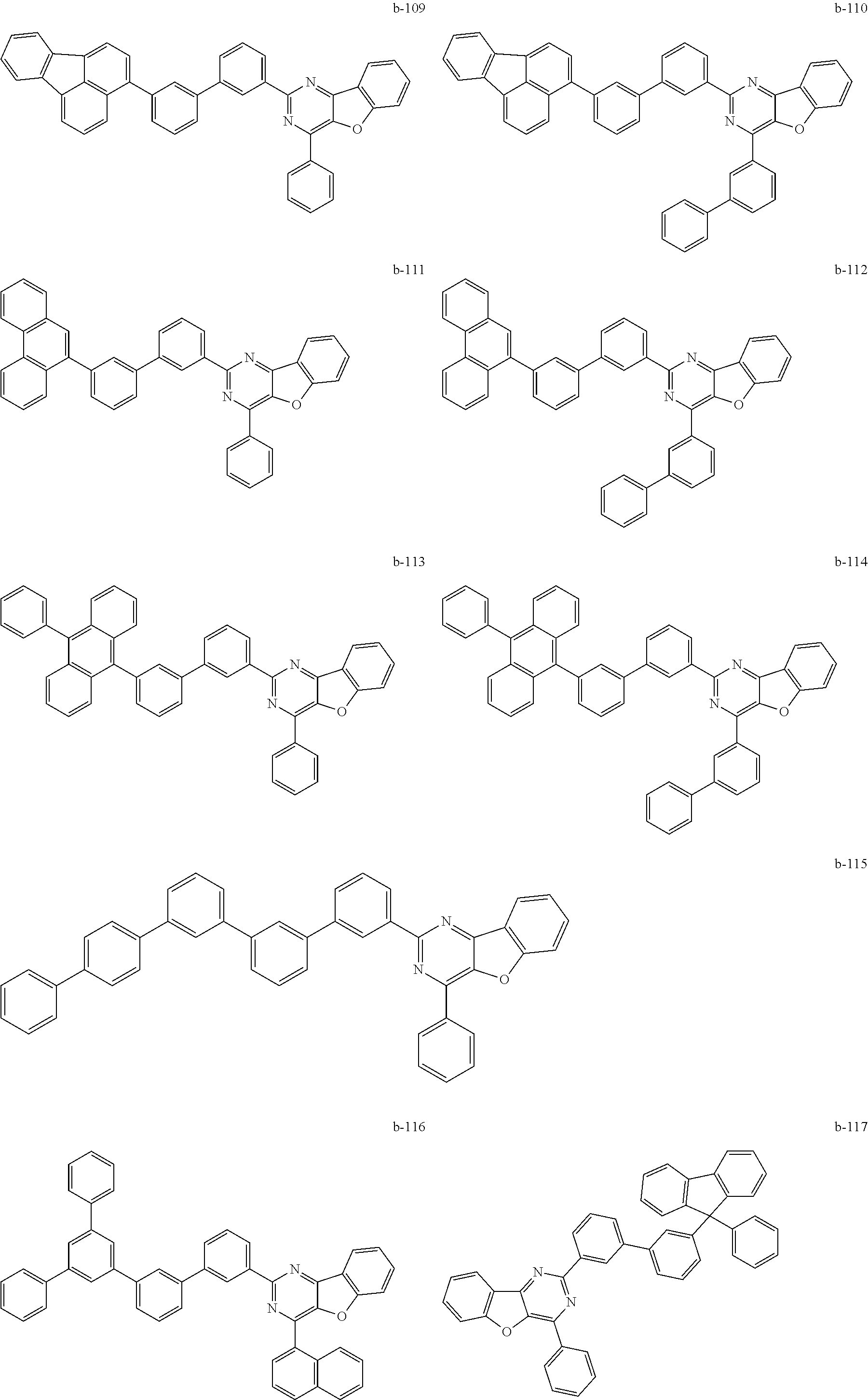

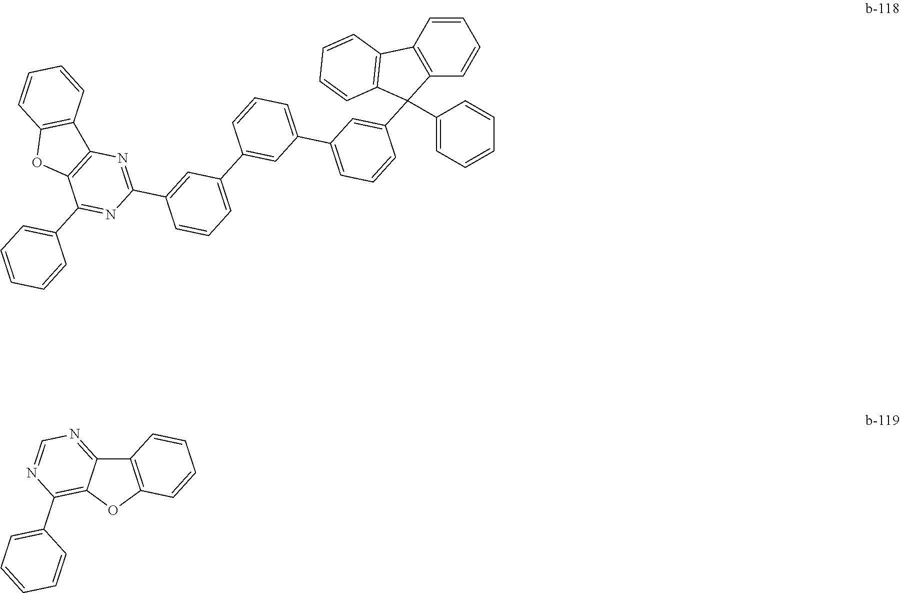 [SMILES: c1ccc(-c2nc(-c3cccc(-c4cccc(-c5cccc(C6(c7ccccc7)c7ccccc7-c7ccccc76)c5)c4)c3)nc3c2oc2ccccc23)cc1 ; c1ccc(-c2ncnc3c2oc2ccccc23)cc1]

Group of X₁=Si(R₄)(R₅) in Formula 1-1

(R₄ and R₅ are described in the present specification)

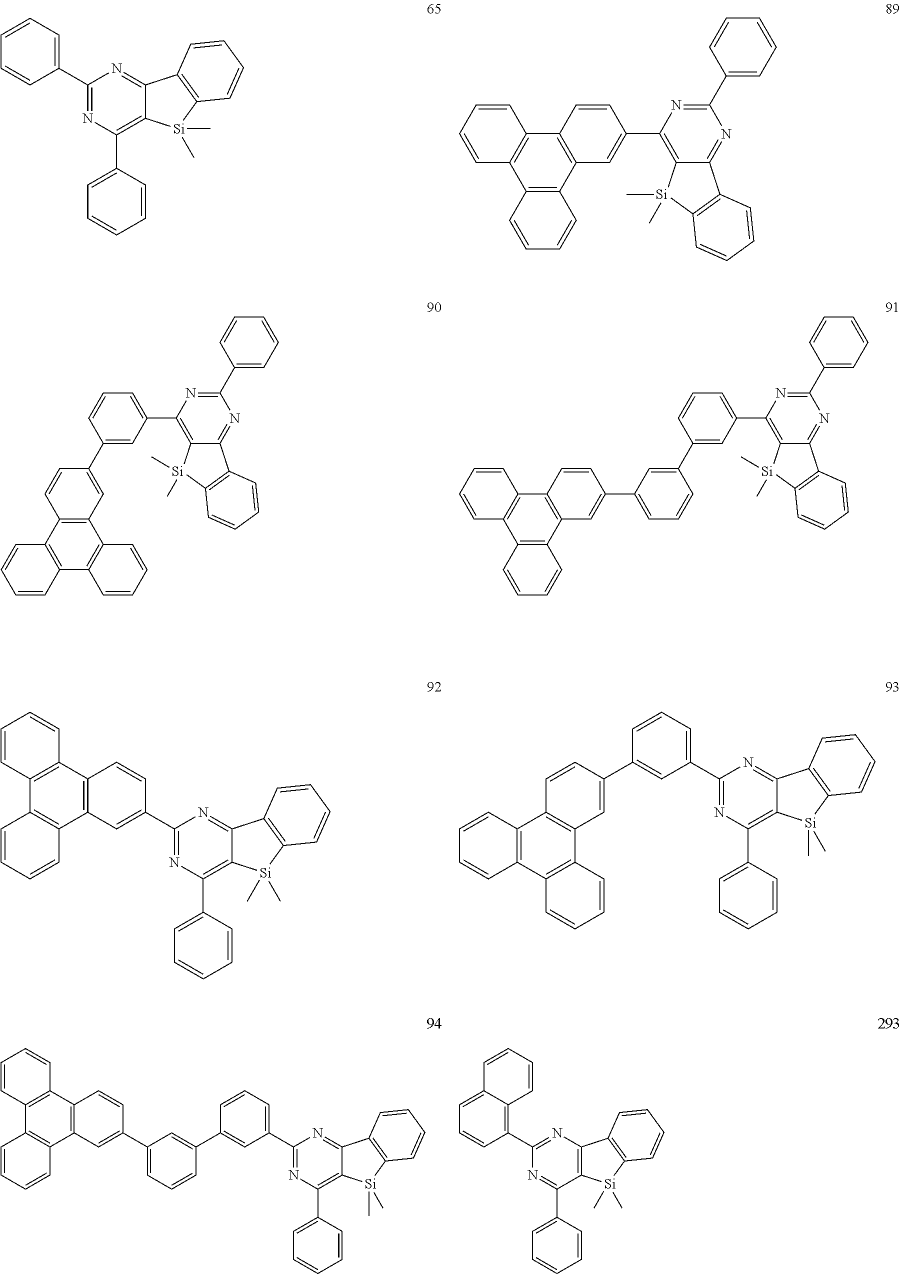

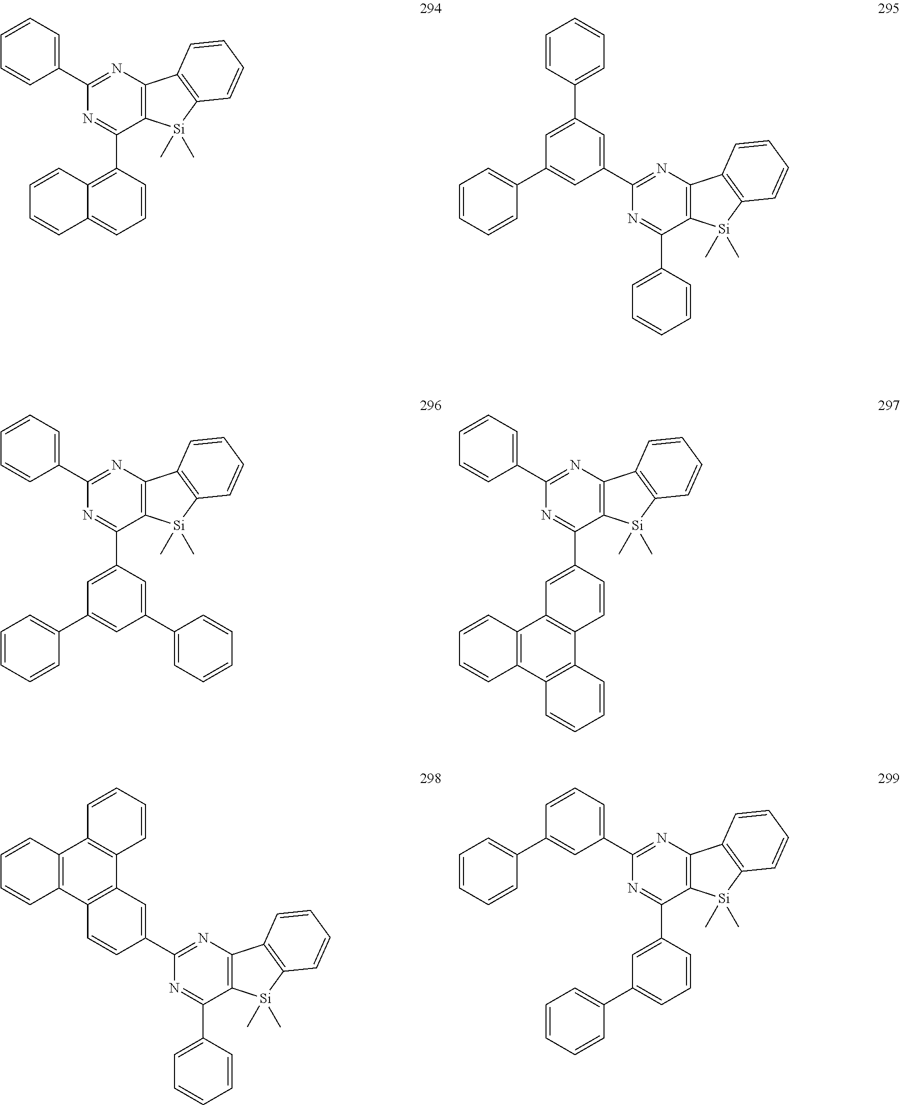 [SMILES: C[Si]1(C)c2ccccc2-c2nc(-c3ccccc3)nc(-c3cccc4ccccc34)c21 ; C[Si]1(C)c2ccccc2-c2nc(-c3cc(-c4ccccc4)cc(-c4ccccc4)c3)nc(-c3ccccc3)c21 ; C[Si]1(C)c2ccccc2-c2nc(-c3ccccc3)nc(-c3cc(-c4ccccc4)cc(-c4ccccc4)c3)c21 ; C[Si]1(C)c2ccccc2-c2nc(-c3ccccc3)nc(-c3ccc4c5ccccc5c5ccccc5c4c3)c21 ; C[Si]1(C)c2ccccc2-c2nc(-c3ccc4c5ccccc5c5ccccc5c4c3)nc(-c3ccccc3)c21 ; C[Si]1(C)c2ccccc2-c2nc(-c3cccc(-c4ccccc4)c3)nc(-c3cccc(-c4ccccc4)c3)c21]

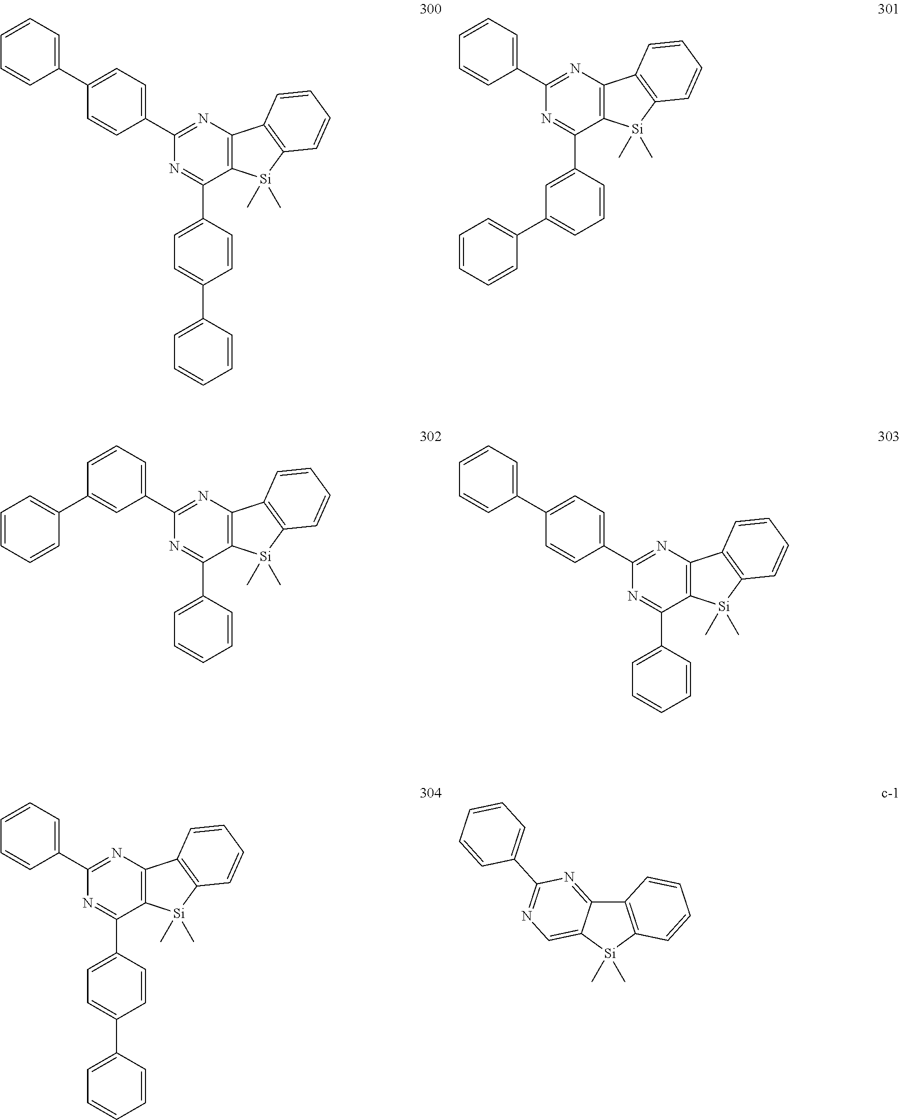 [SMILES: C[Si]1(C)c2ccccc2-c2nc(-c3ccc(-c4ccccc4)cc3)nc(-c3ccc(-c4ccccc4)cc3)c21 ; C[Si]1(C)c2ccccc2-c2nc(-c3ccccc3)nc(-c3cccc(-c4ccccc4)c3)c21 ; C[Si]1(C)c2ccccc2-c2nc(-c3cccc(-c4ccccc4)c3)nc(-c3ccccc3)c21 ; C[Si]1(C)c2ccccc2-c2nc(-c3ccc(-c4ccccc4)cc3)nc(-c3ccccc3)c21 ; C[Si]1(C)c2ccccc2-c2nc(-c3ccccc3)nc(-c3ccc(-c4ccccc4)cc3)c21 ; C[Si]1(C)c2ccccc2-c2nc(-c3ccccc3)ncc21]

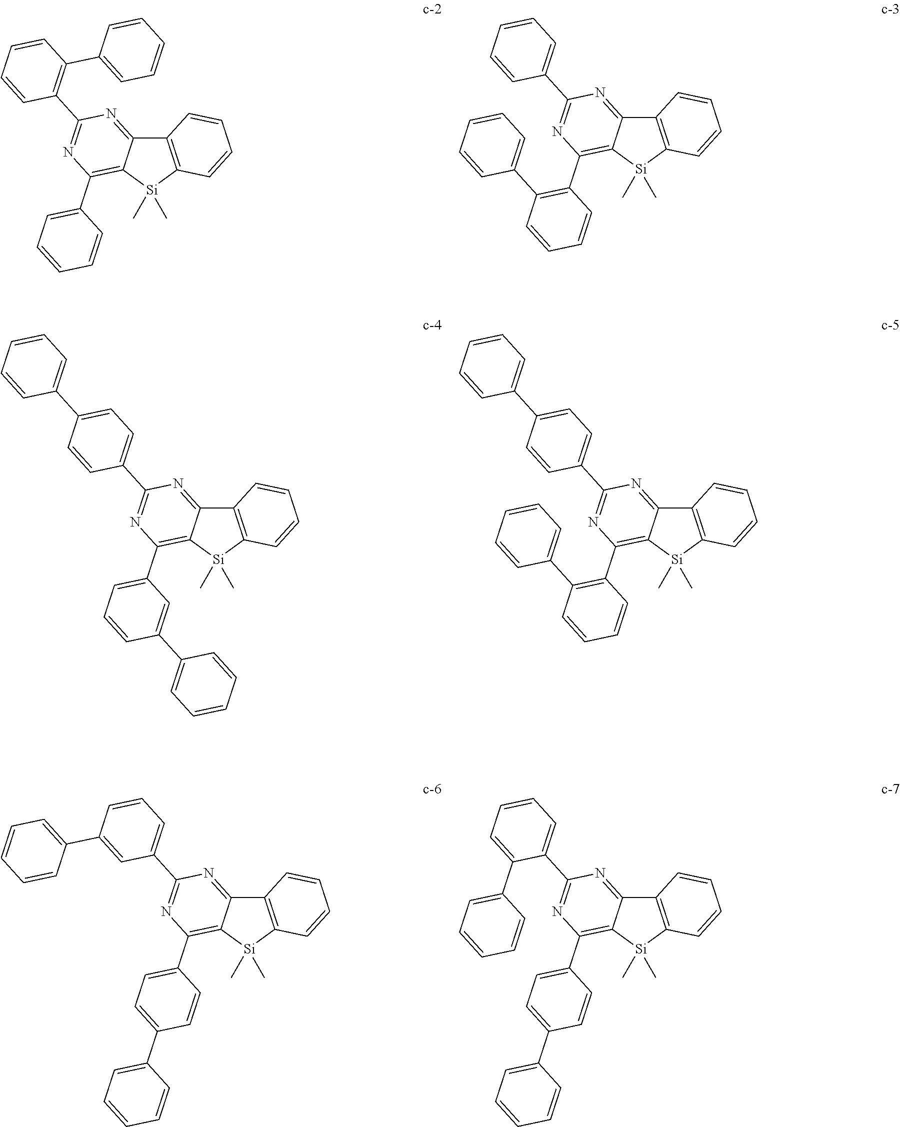 [SMILES: C[Si]1(C)c2ccccc2-c2nc(-c3ccccc3-c3ccccc3)nc(-c3ccccc3)c21 ; C[Si]1(C)c2ccccc2-c2nc(-c3ccccc3)nc(-c3ccccc3-c3ccccc3)c21 ; C[Si]1(C)c2ccccc2-c2nc(-c3ccc(-c4ccccc4)cc3)nc(-c3cccc(-c4ccccc4)c3)c21 ; C[Si]1(C)c2ccccc2-c2nc(-c3ccc(-c4ccccc4)cc3)nc(-c3ccccc3-c3ccccc3)c21 ; C[Si]1(C)c2ccccc2-c2nc(-c3cccc(-c4ccccc4)c3)nc(-c3ccc(-c4ccccc4)cc3)c21 ; C[Si]1(C)c2ccccc2-c2nc(-c3ccccc3-c3ccccc3)nc(-c3ccc(-c4ccccc4)cc3)c21]

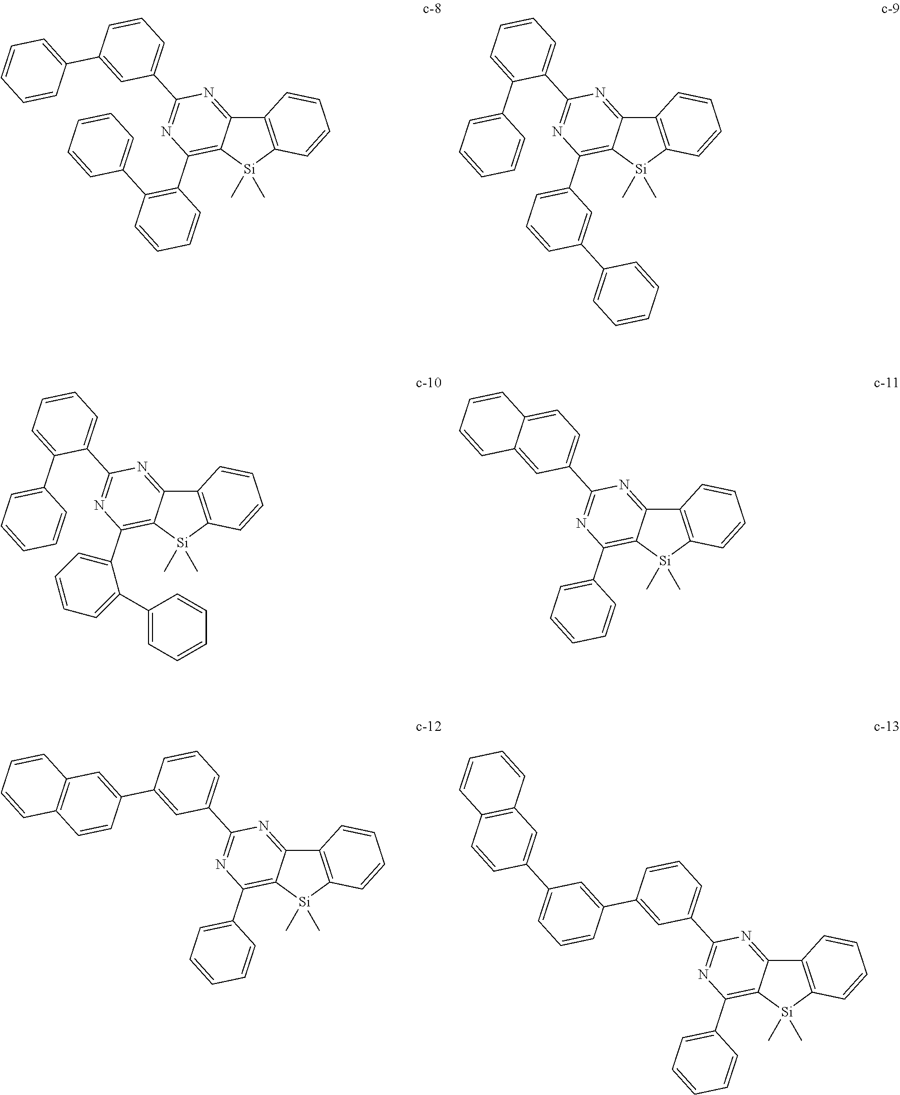 [SMILES: C[Si]1(C)c2ccccc2-c2nc(-c3cccc(-c4ccccc4)c3)nc(-c3ccccc3-c3ccccc3)c21 ; C[Si]1(C)c2ccccc2-c2nc(-c3ccccc3-c3ccccc3)nc(-c3cccc(-c4ccccc4)c3)c21 ; C[Si]1(C)c2ccccc2-c2nc(-c3ccccc3-c3ccccc3)nc(-c3ccccc3-c3ccccc3)c21 ; C[Si]1(C)c2ccccc2-c2nc(-c3ccc4ccccc4c3)nc(-c3ccccc3)c21 ; C[Si]1(C)c2ccccc2-c2nc(-c3cccc(-c4ccc5ccccc5c4)c3)nc(-c3ccccc3)c21 ; C[Si]1(C)c2ccccc2-c2nc(-c3cccc(-c4cccc(-c5ccc6ccccc6c5)c4)c3)nc(-c3ccccc3)c21]

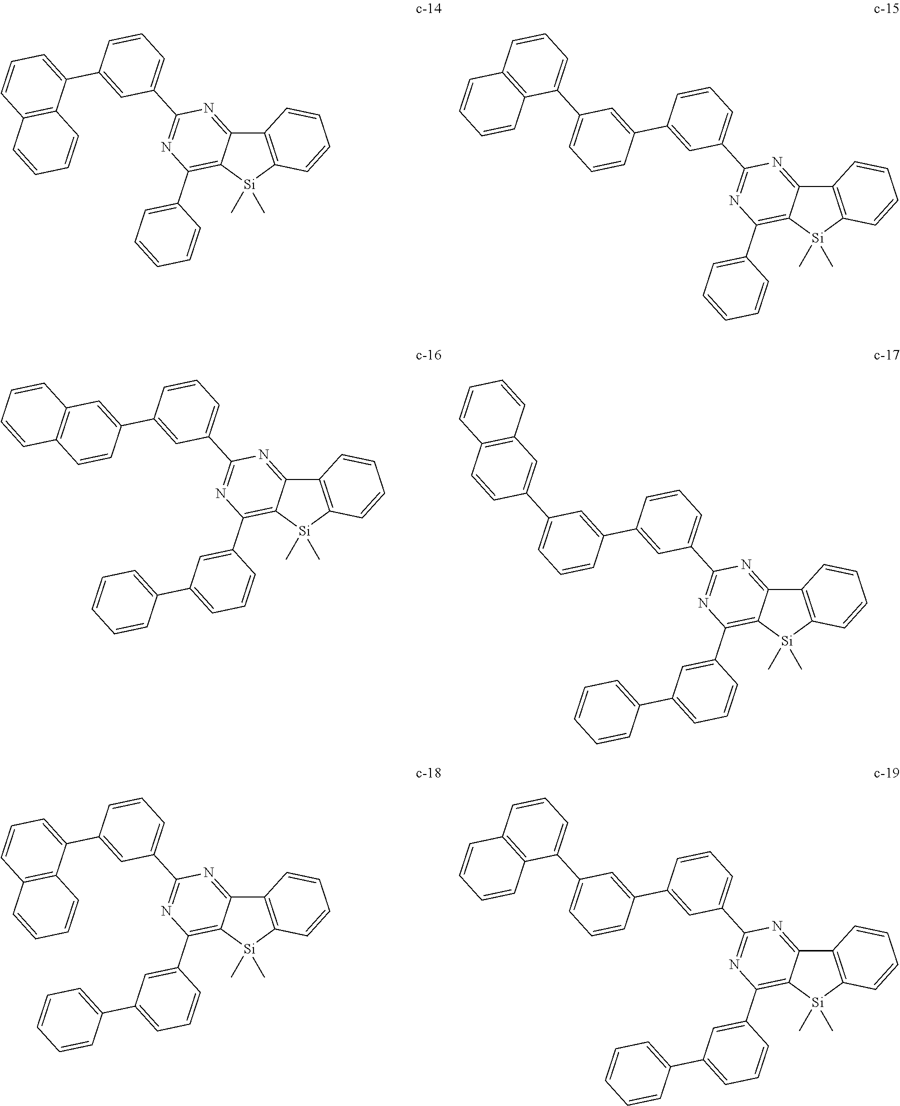 [SMILES: C[Si]1(C)c2ccccc2-c2nc(-c3cccc(-c4cccc5ccccc45)c3)nc(-c3ccccc3)c21 ; C[Si]1(C)c2ccccc2-c2nc(-c3cccc(-c4cccc(-c5cccc6ccccc56)c4)c3)nc(-c3ccccc3)c21 ; C[Si]1(C)c2ccccc2-c2nc(-c3cccc(-c4ccc5ccccc5c4)c3)nc(-c3cccc(-c4ccccc4)c3)c21 ; C[Si]1(C)c2ccccc2-c2nc(-c3cccc(-c4cccc(-c5ccc6ccccc6c5)c4)c3)nc(-c3cccc(-c4ccccc4)c3)c21 ; C[Si]1(C)c2ccccc2-c2nc(-c3cccc(-c4cccc5ccccc45)c3)nc(-c3cccc(-c4ccccc4)c3)c21 ; C[Si]1(C)c2ccccc2-c2nc(-c3cccc(-c4cccc(-c5cccc6ccccc56)c4)c3)nc(-c3cccc(-c4ccccc4)c3)c21]

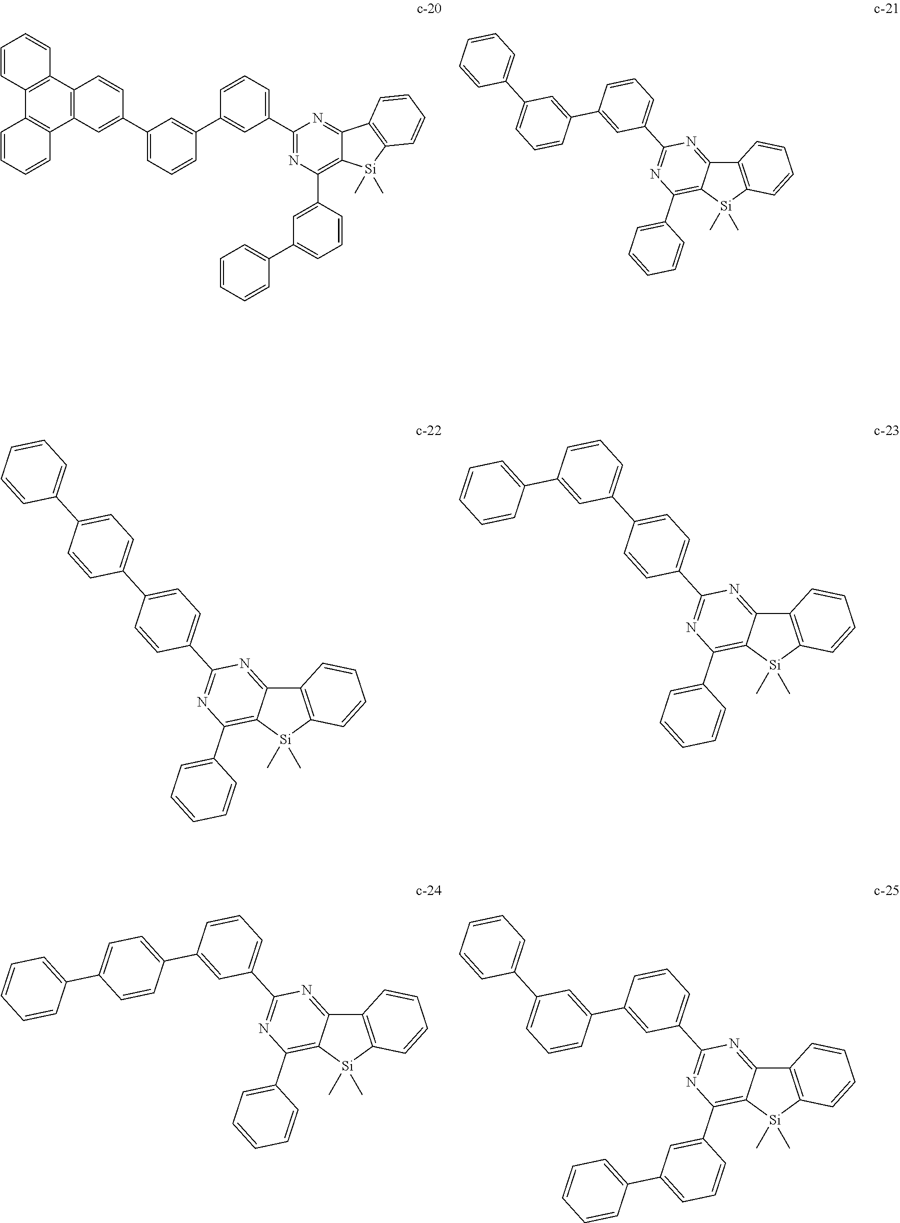 [SMILES: C[Si]1(C)c2ccccc2-c2nc(-c3cccc(-c4cccc(-c5ccc6c7ccccc7c7ccccc7c6c5)c4)c3)nc(-c3cccc(-c4ccccc4)c3)c21 ; C[Si]1(C)c2ccccc2-c2nc(-c3cccc(-c4cccc(-c5ccccc5)c4)c3)nc(-c3ccccc3)c21 ; C[Si]1(C)c2ccccc2-c2nc(-c3ccc(-c4ccc(-c5ccccc5)cc4)cc3)nc(-c3ccccc3)c21 ; C[Si]1(C)c2ccccc2-c2nc(-c3ccc(-c4cccc(-c5ccccc5)c4)cc3)nc(-c3ccccc3)c21 ; C[Si]1(C)c2ccccc2-c2nc(-c3cccc(-c4ccc(-c5ccccc5)cc4)c3)nc(-c3ccccc3)c21 ; C[Si]1(C)c2ccccc2-c2nc(-c3cccc(-c4cccc(-c5ccccc5)c4)c3)nc(-c3cccc(-c4ccccc4)c3)c21]

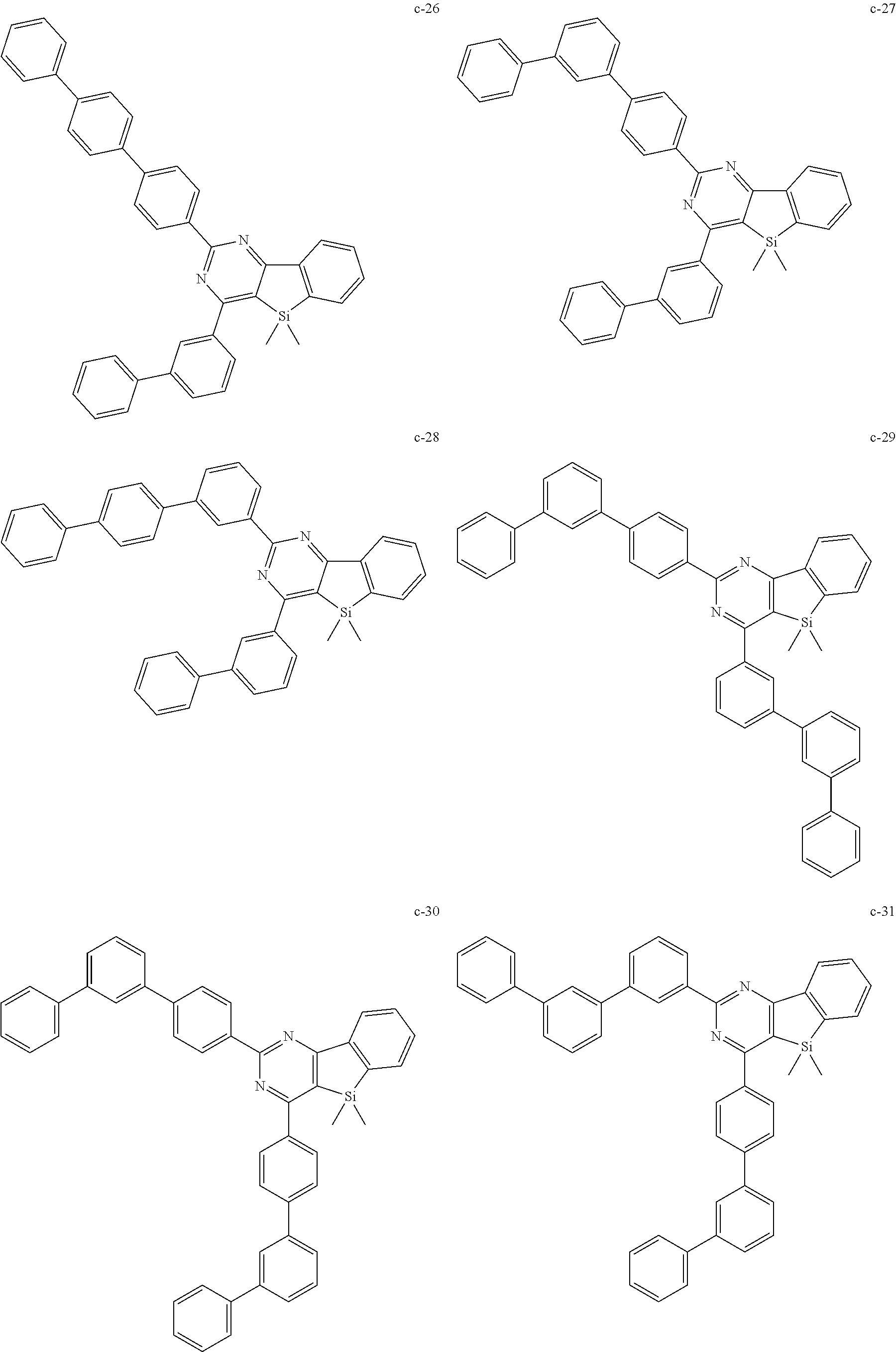

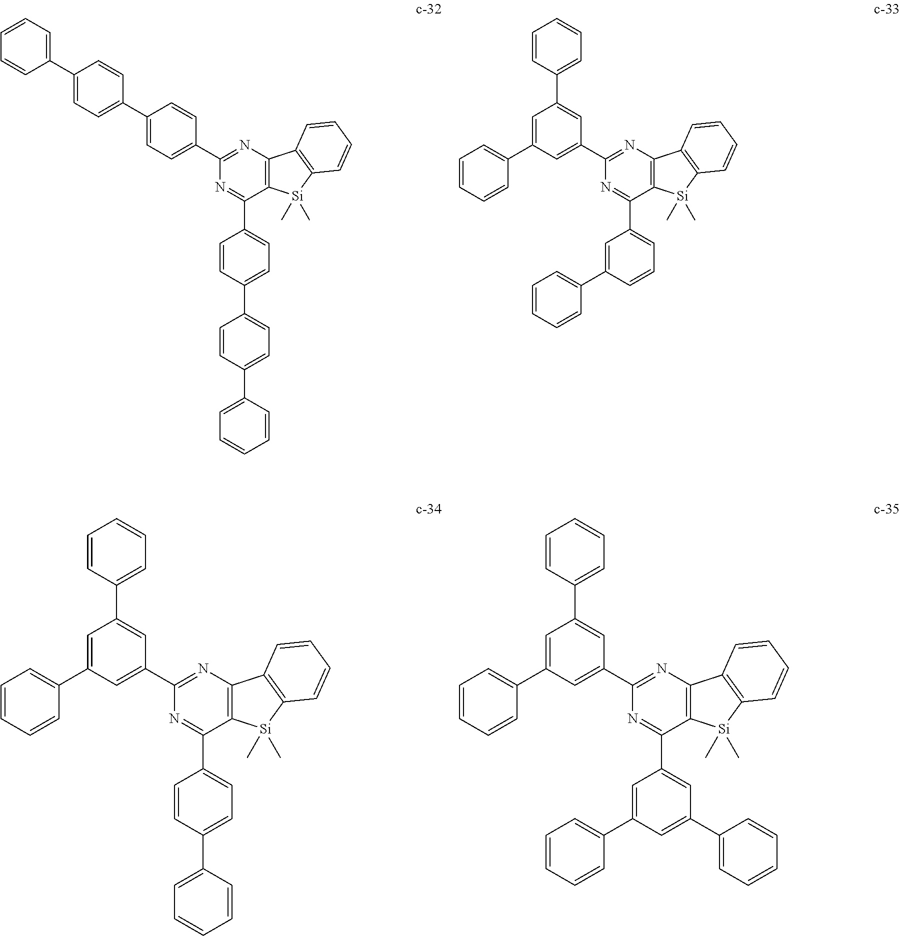 [SMILES: C[Si]1(C)c2ccccc2-c2nc(-c3ccc(-c4ccc(-c5ccccc5)cc4)cc3)nc(-c3ccc(-c4ccc(-c5ccccc5)cc4)cc3)c21 ; C[Si]1(C)c2ccccc2-c2nc(-c3cc(-c4ccccc4)cc(-c4ccccc4)c3)nc(-c3cccc(-c4ccccc4)c3)c21 ; C[Si]1(C)c2ccccc2-c2nc(-c3cc(-c4ccccc4)cc(-c4ccccc4)c3)nc(-c3ccc(-c4ccccc4)cc3)c21 ; C[Si]1(C)c2ccccc2-c2nc(-c3cc(-c4ccccc4)cc(-c4ccccc4)c3)nc(-c3cc(-c4ccccc4)cc(-c4ccccc4)c3)c21]

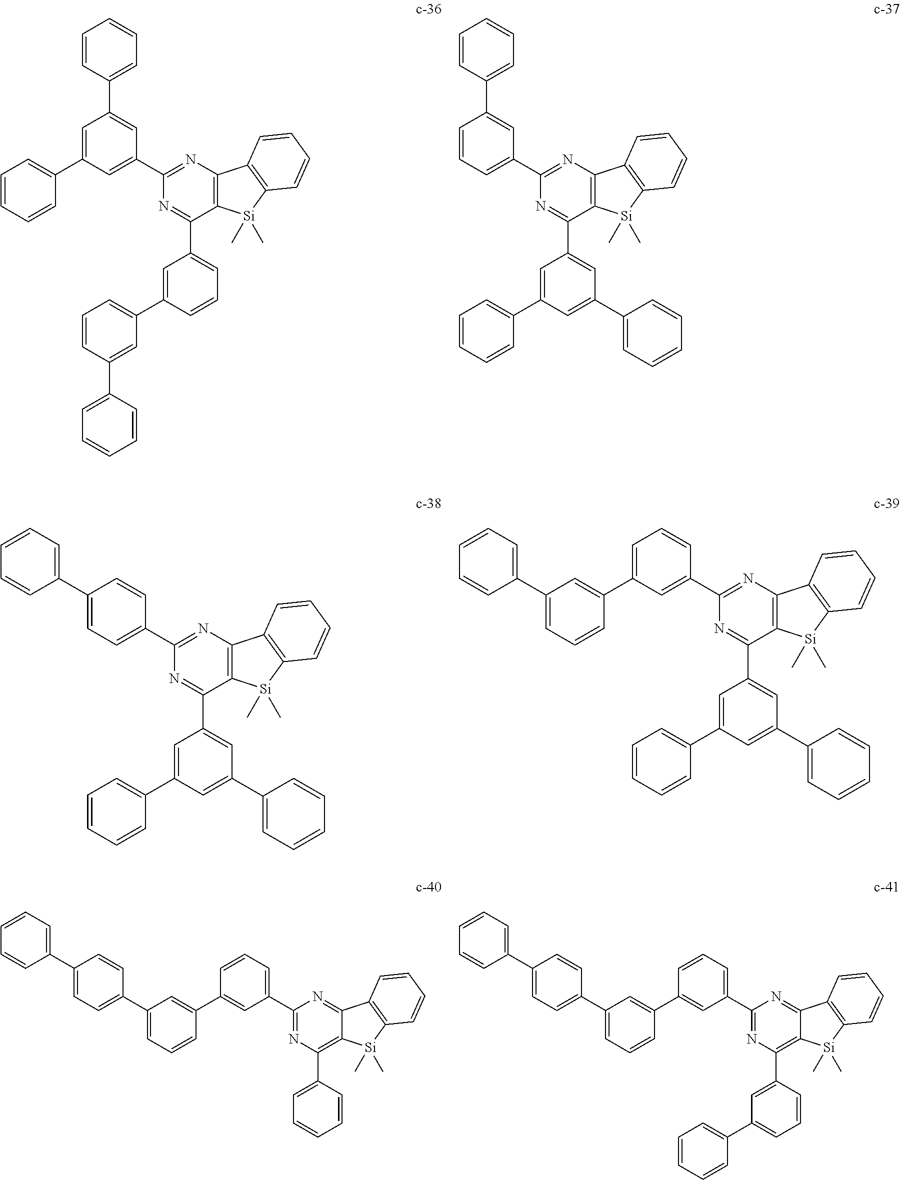

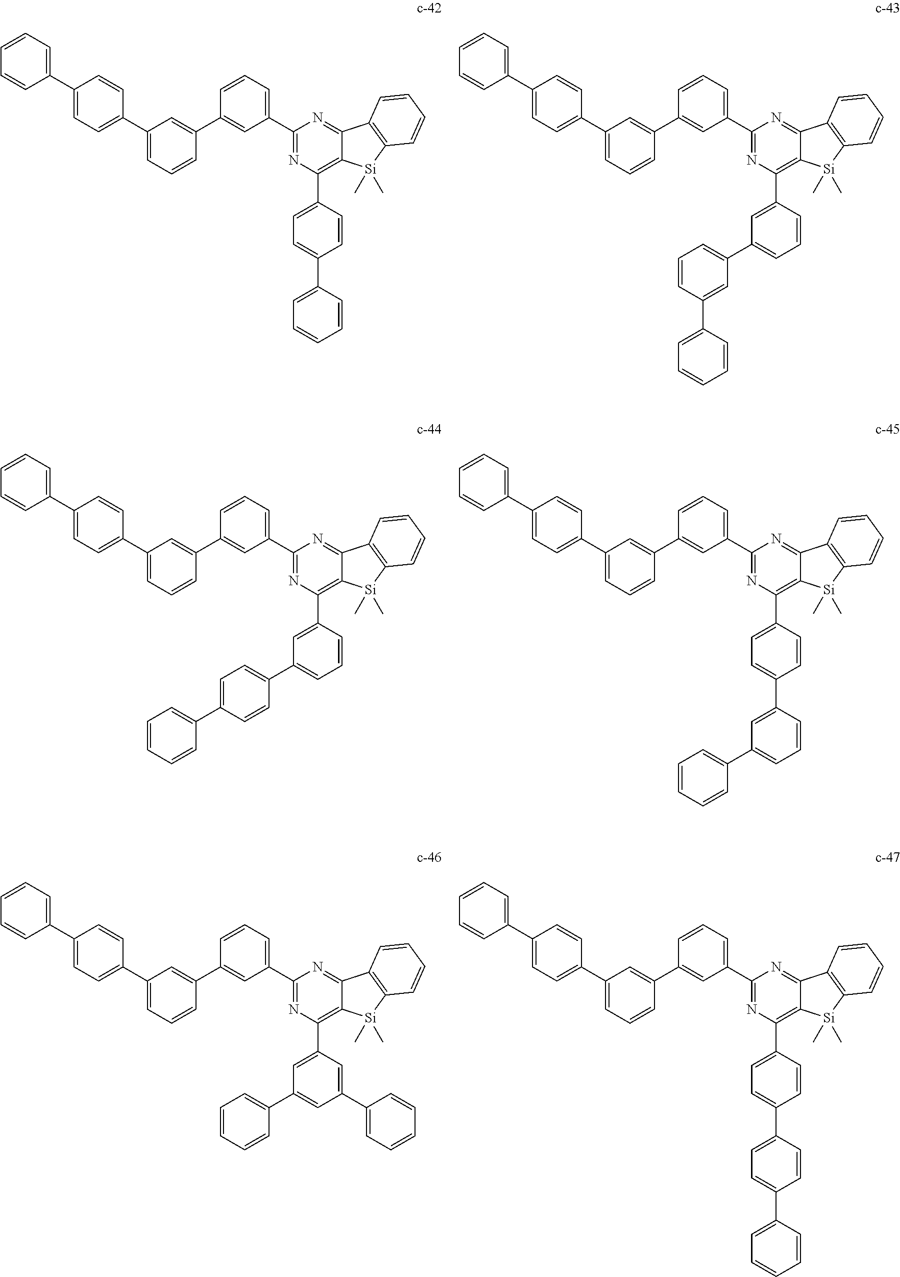

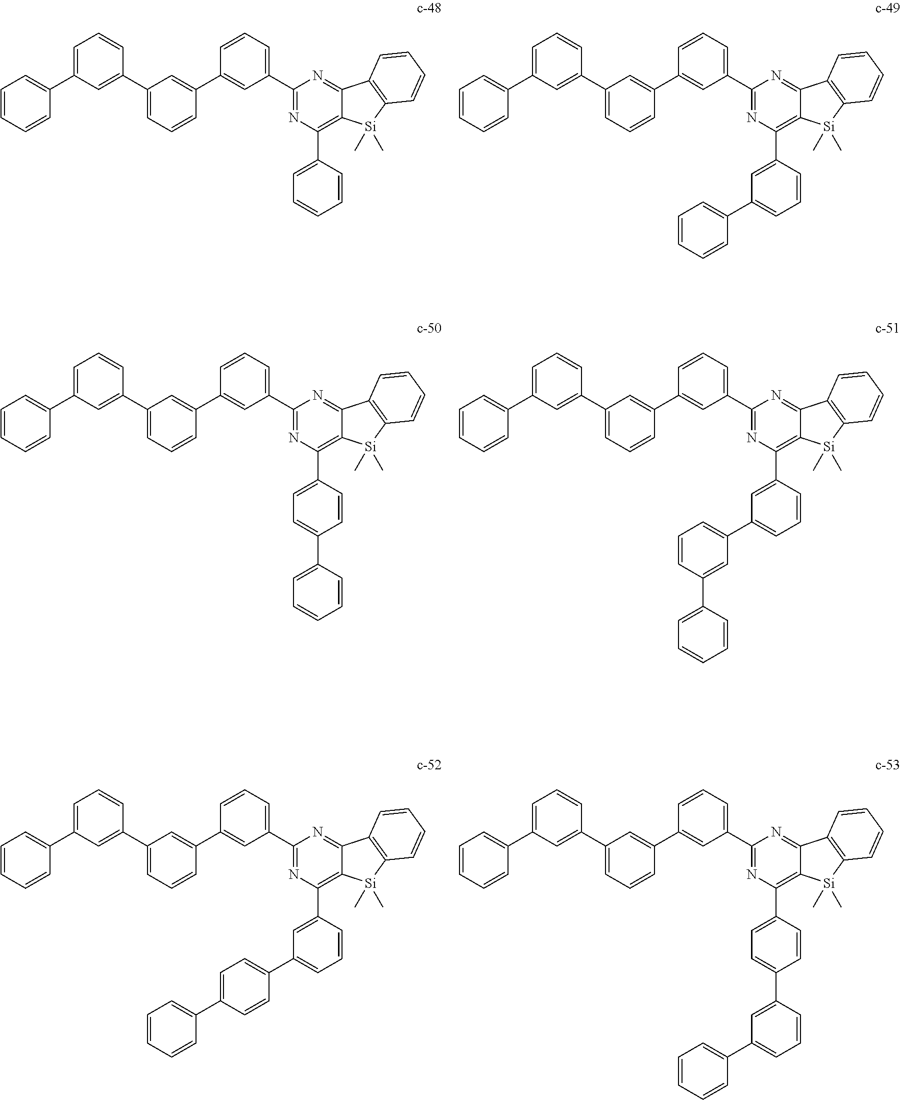

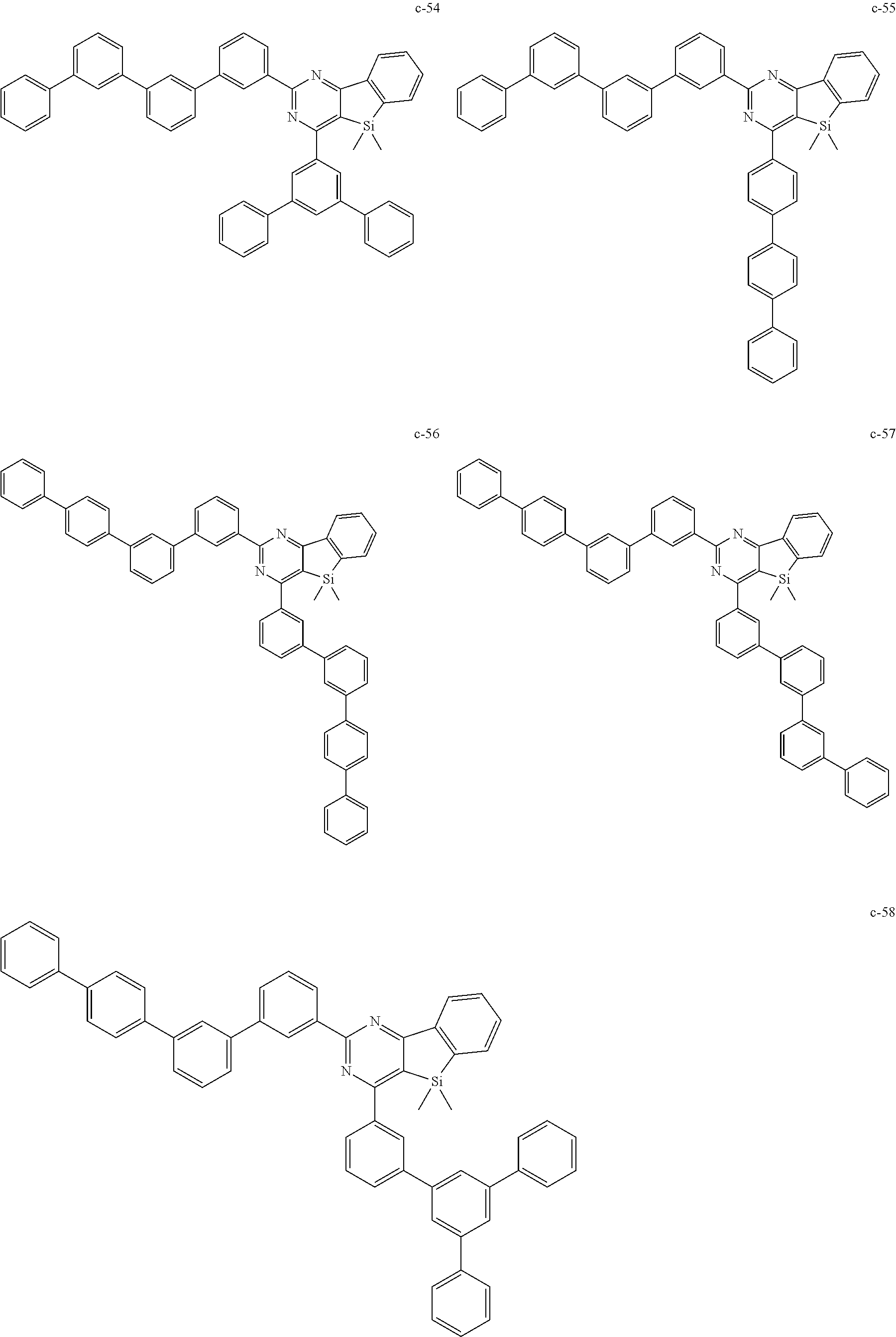

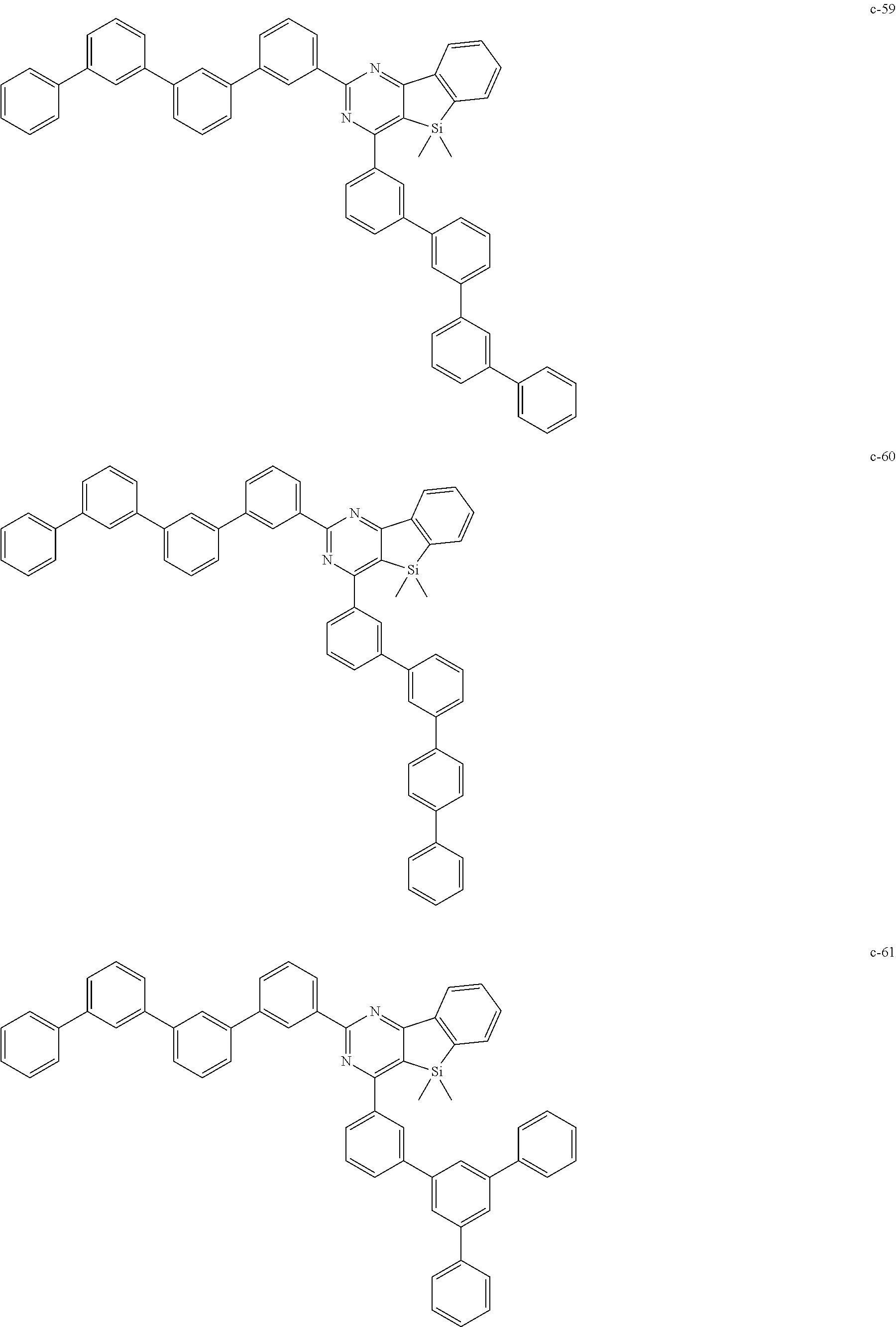 [SMILES: C[Si]1(C)c2ccccc2-c2nc(-c3cccc(-c4cccc(-c5cccc(-c6ccccc6)c5)c4)c3)nc(-c3cccc(-c4cccc(-c5cccc(-c6ccccc6)c5)c4)c3)c21 ; C[Si]1(C)c2ccccc2-c2nc(-c3cccc(-c4cccc(-c5cccc(-c6ccccc6)c5)c4)c3)nc(-c3cccc(-c4cccc(-c5ccc(-c6ccccc6)cc5)c4)c3)c21 ; C[Si]1(C)c2ccccc2-c2nc(-c3cccc(-c4cccc(-c5cccc(-c6ccccc6)c5)c4)c3)nc(-c3cccc(-c4cc(-c5ccccc5)cc(-c5ccccc5)c4)c3)c21]

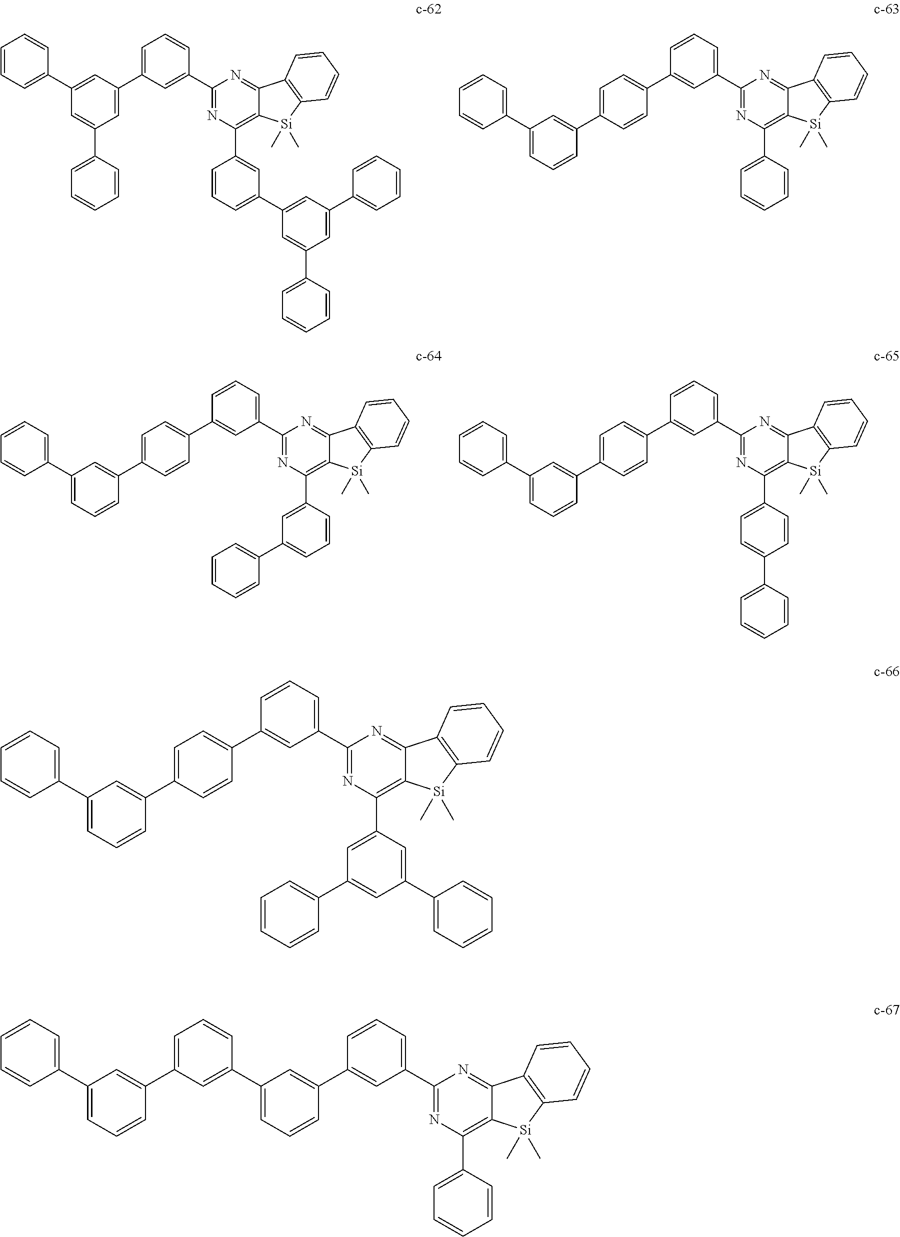

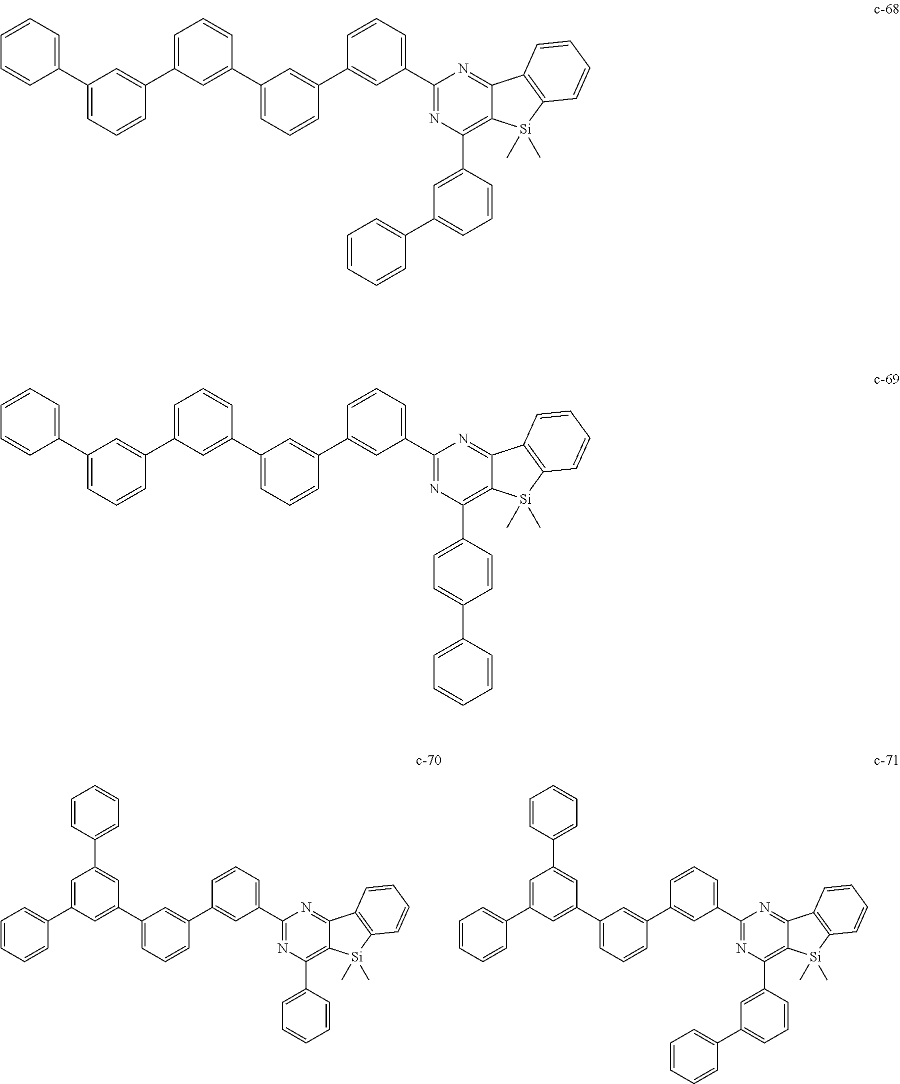 [SMILES: C[Si]1(C)c2ccccc2-c2nc(-c3cccc(-c4cccc(-c5cccc(-c6cccc(-c7ccccc7)c6)c5)c4)c3)nc(-c3cccc(-c4ccccc4)c3)c21 ; C[Si]1(C)c2ccccc2-c2nc(-c3cccc(-c4cccc(-c5cccc(-c6cccc(-c7ccccc7)c6)c5)c4)c3)nc(-c3ccc(-c4ccccc4)cc3)c21 ; C[Si]1(C)c2ccccc2-c2nc(-c3cccc(-c4cccc(-c5cc(-c6ccccc6)cc(-c6ccccc6)c5)c4)c3)nc(-c3ccccc3)c21 ; C[Si]1(C)c2ccccc2-c2nc(-c3cccc(-c4cccc(-c5cc(-c6ccccc6)cc(-c6ccccc6)c5)c4)c3)nc(-c3cccc(-c4ccccc4)c3)c21]

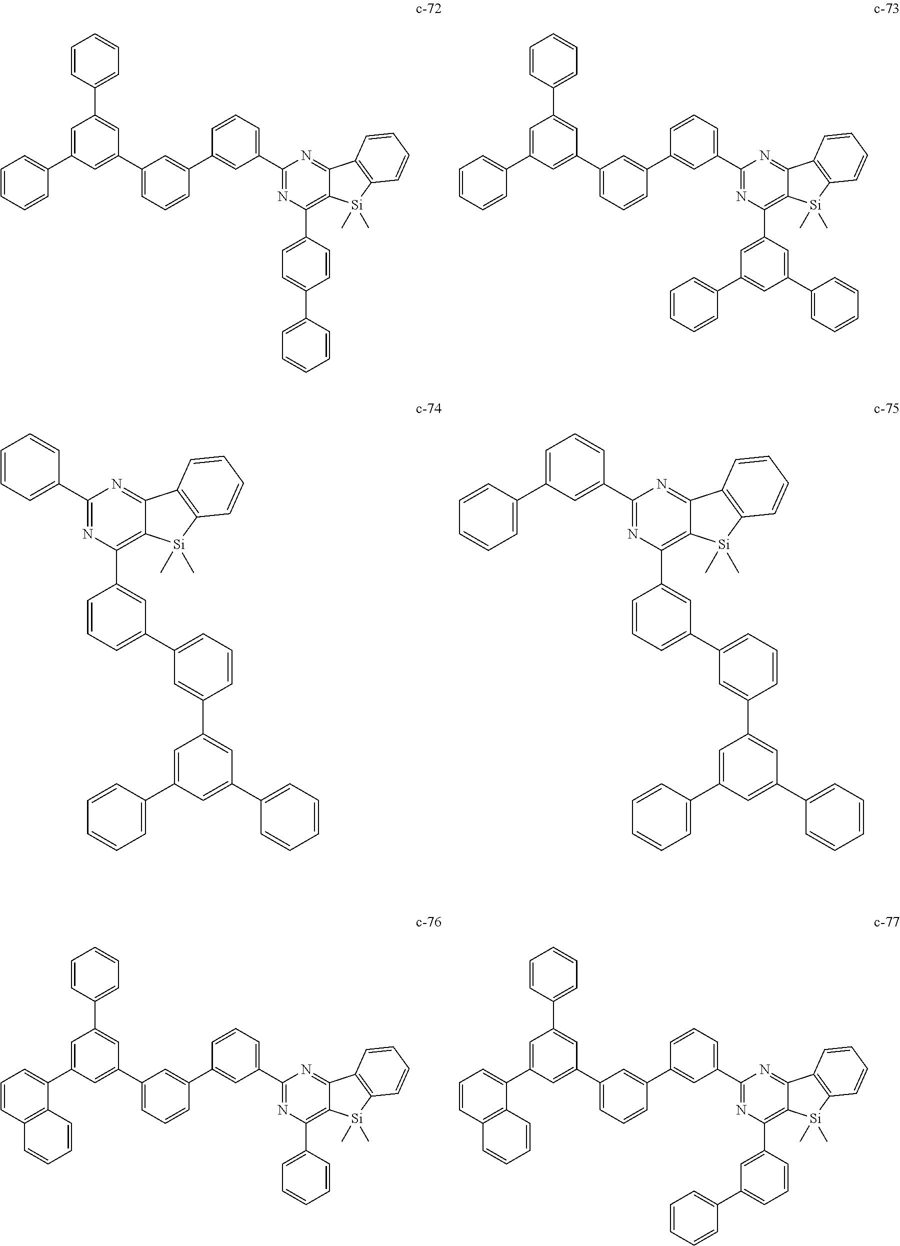

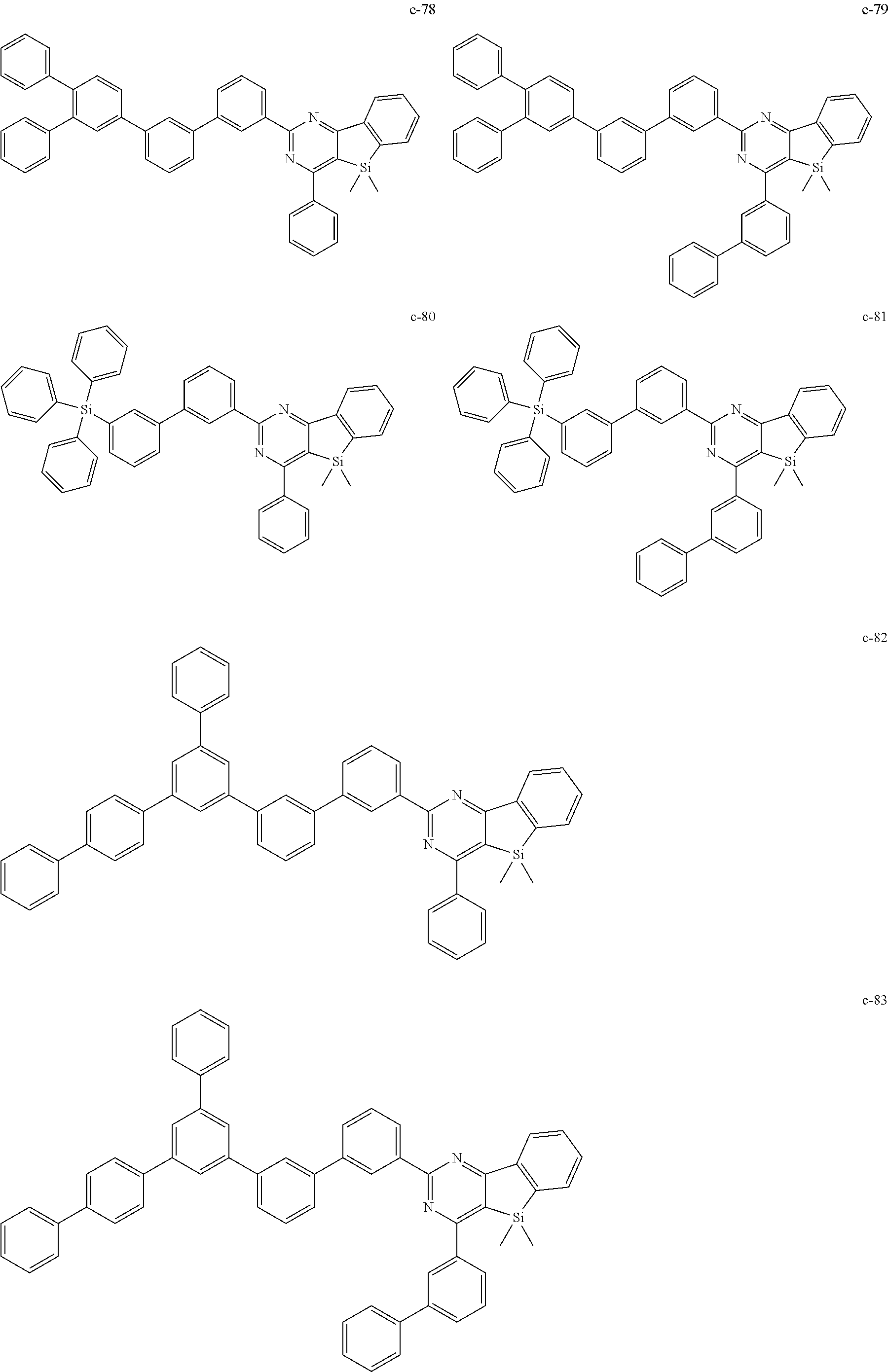

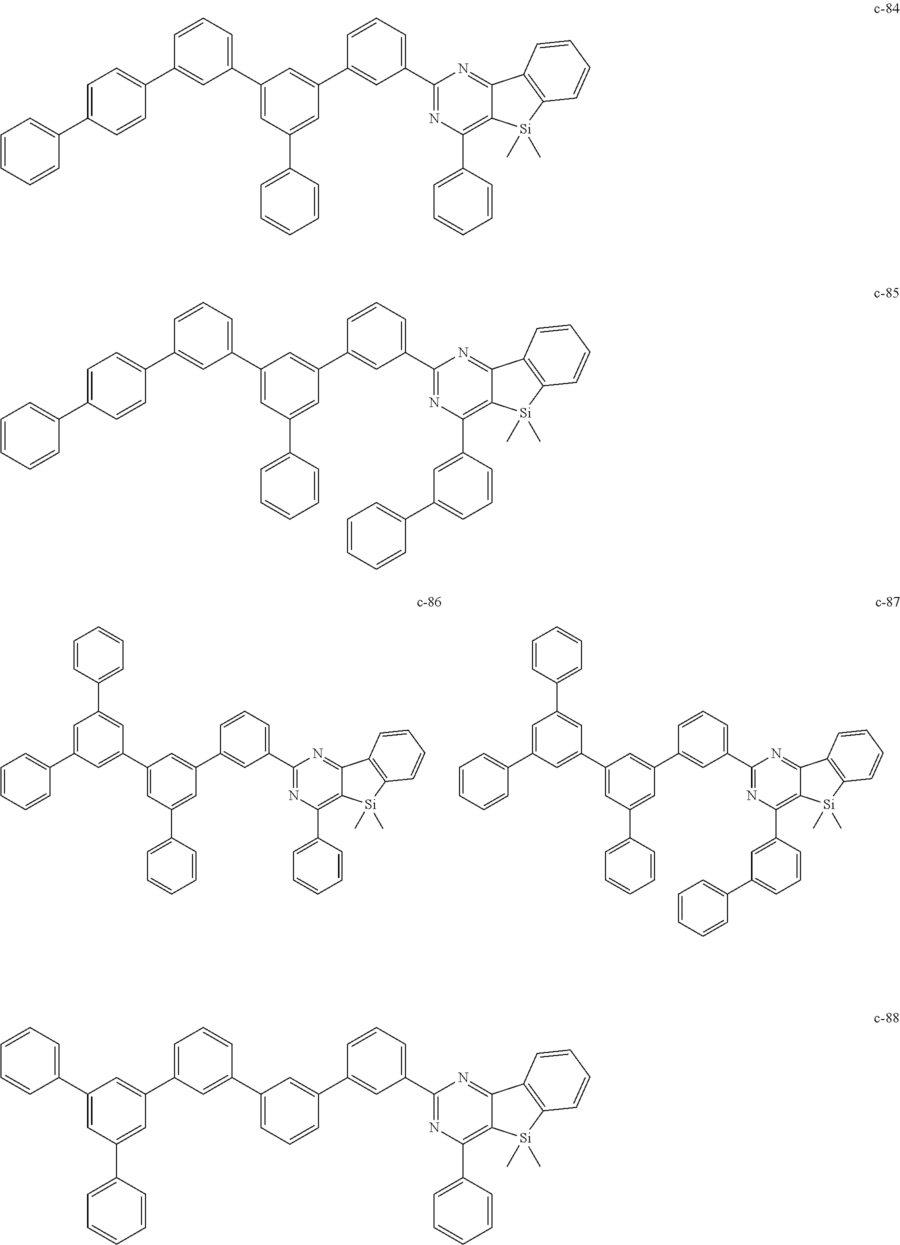

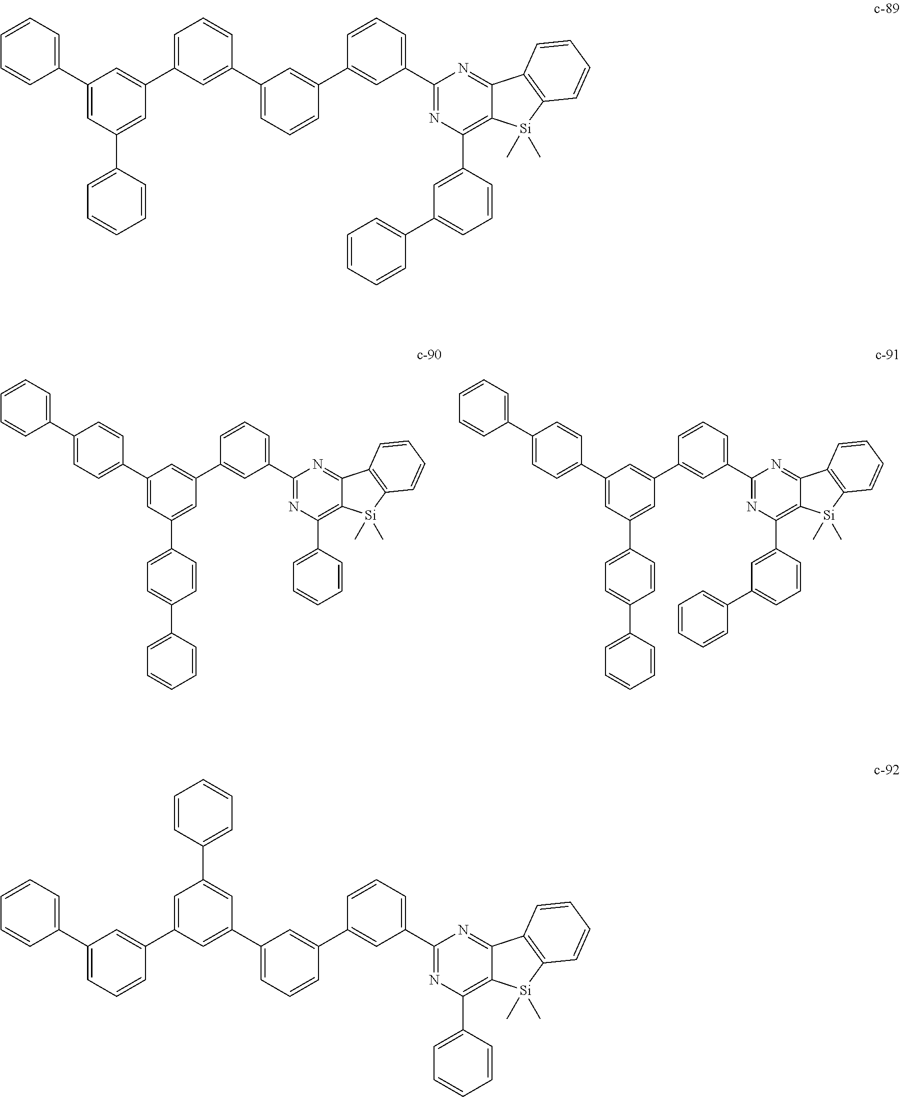 [SMILES: C[Si]1(C)c2ccccc2-c2nc(-c3cccc(-c4cccc(-c5cccc(-c6cc(-c7ccccc7)cc(-c7ccccc7)c6)c5)c4)c3)nc(-c3cccc(-c4ccccc4)c3)c21 ; C[Si]1(C)c2ccccc2-c2nc(-c3cccc(-c4cc(-c5ccc(-c6ccccc6)cc5)cc(-c5ccc(-c6ccccc6)cc5)c4)c3)nc(-c3ccccc3)c21 ; C[Si]1(C)c2ccccc2-c2nc(-c3cccc(-c4cc(-c5ccc(-c6ccccc6)cc5)cc(-c5ccc(-c6ccccc6)cc5)c4)c3)nc(-c3cccc(-c4ccccc4)c3)c21 ; C[Si]1(C)c2ccccc2-c2nc(-c3cccc(-c4cccc(-c5cc(-c6ccccc6)cc(-c6cccc(-c7ccccc7)c6)c5)c4)c3)nc(-c3ccccc3)c21]

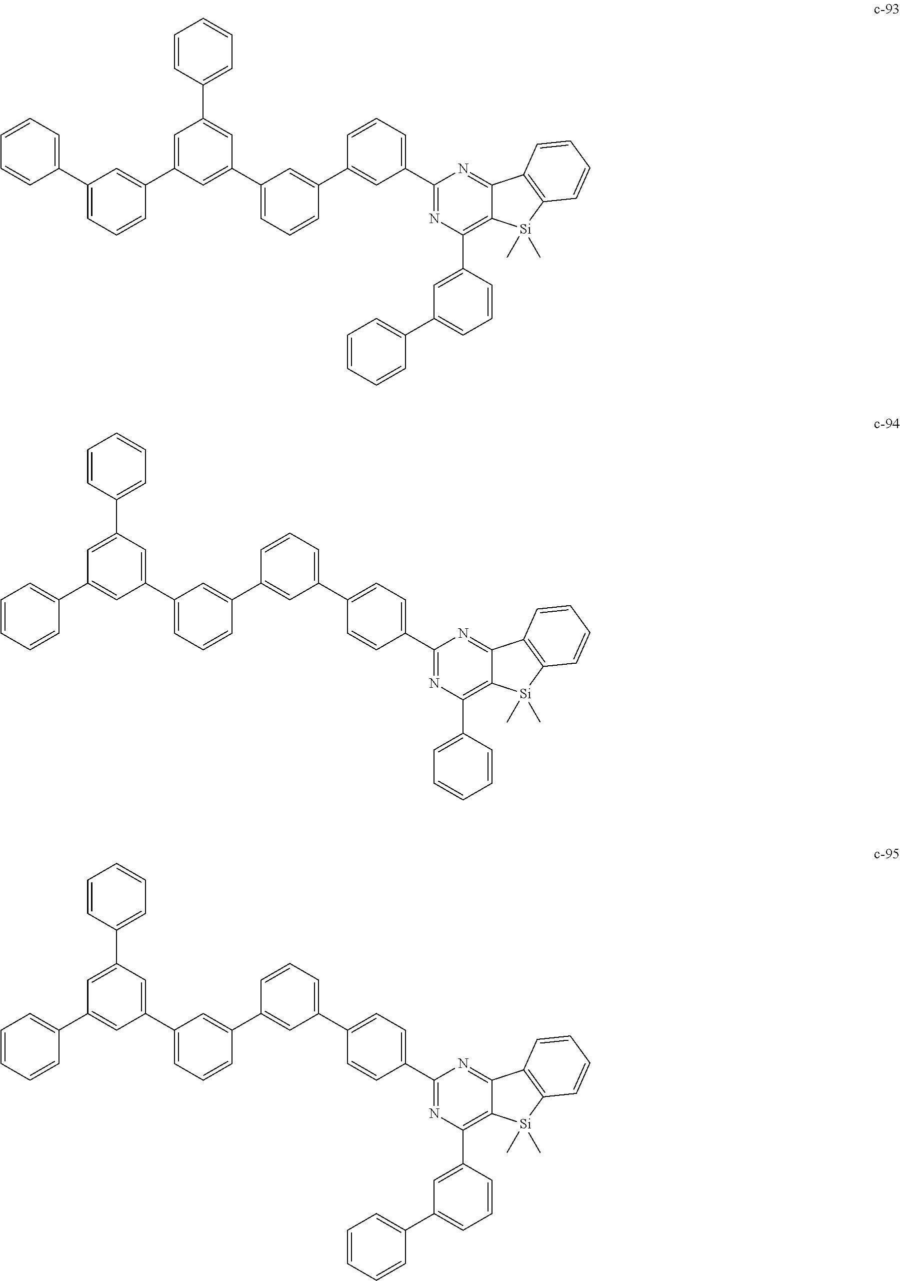 [SMILES: C[Si]1(C)c2ccccc2-c2nc(-c3cccc(-c4cccc(-c5cc(-c6ccccc6)cc(-c6cccc(-c7ccccc7)c6)c5)c4)c3)nc(-c3cccc(-c4ccccc4)c3)c21 ; C[Si]1(C)c2ccccc2-c2nc(-c3ccc(-c4cccc(-c5cccc(-c6cc(-c7ccccc7)cc(-c7ccccc7)c6)c5)c4)cc3)nc(-c3ccccc3)c21 ; C[Si]1(C)c2ccccc2-c2nc(-c3ccc(-c4cccc(-c5cccc(-c6cc(-c7ccccc7)cc(-c7ccccc7)c6)c5)c4)cc3)nc(-c3cccc(-c4ccccc4)c3)c21]

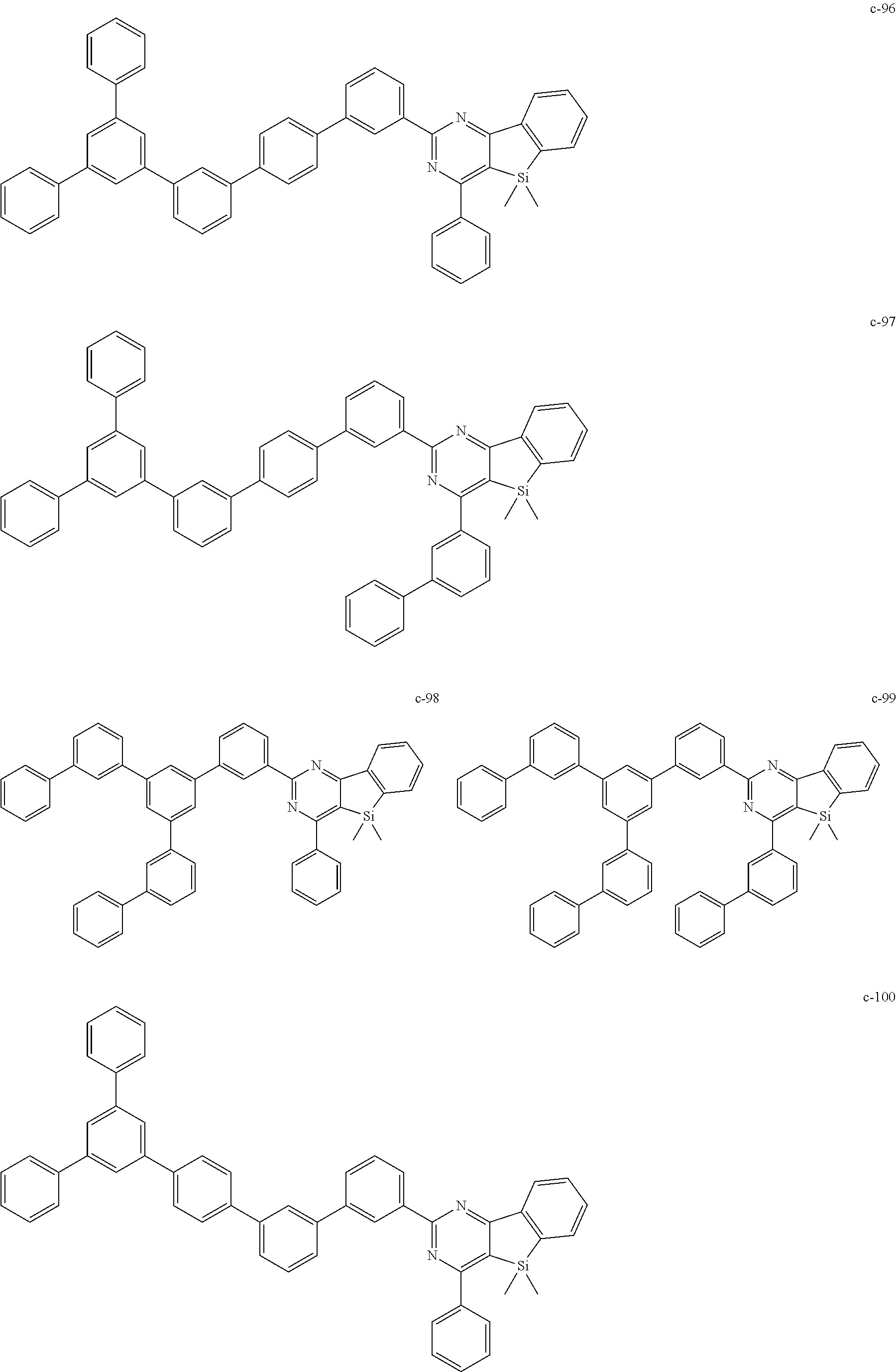

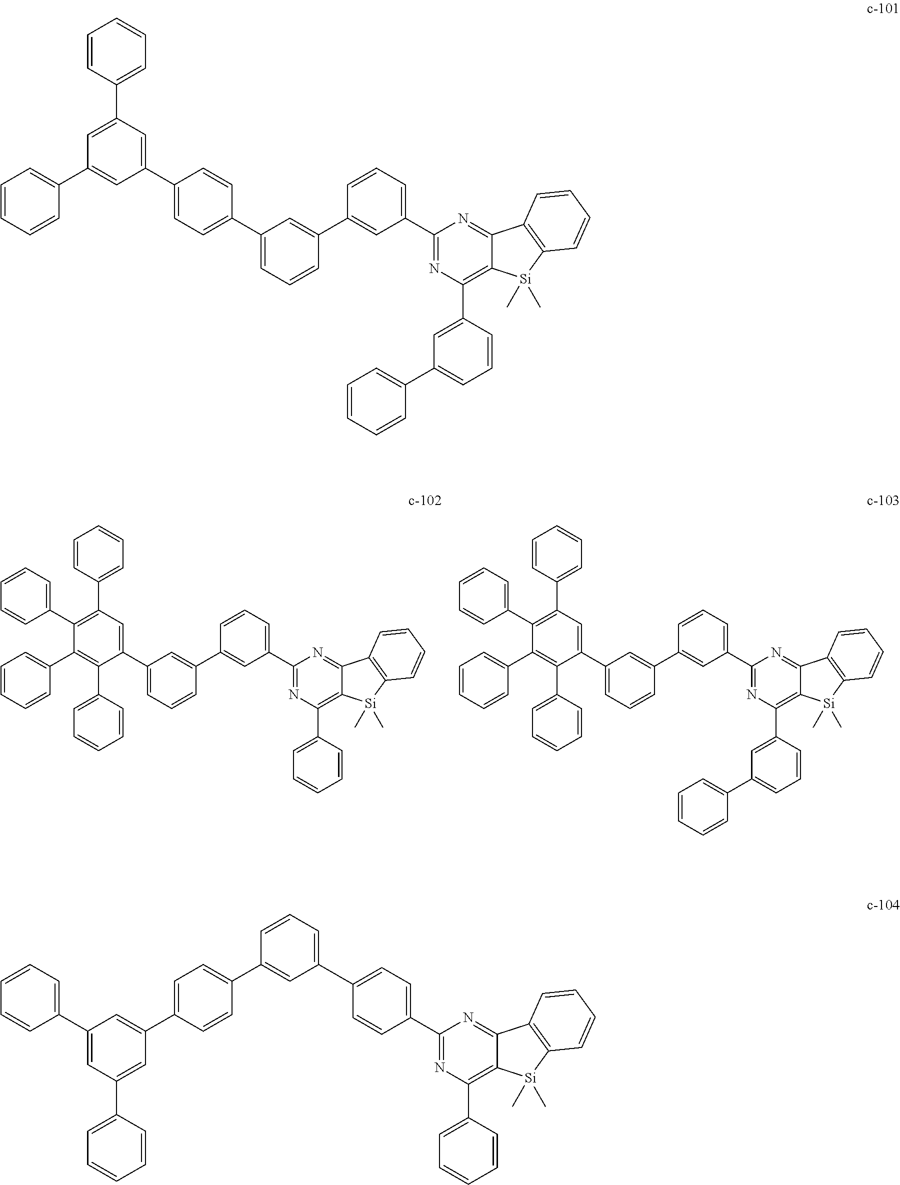

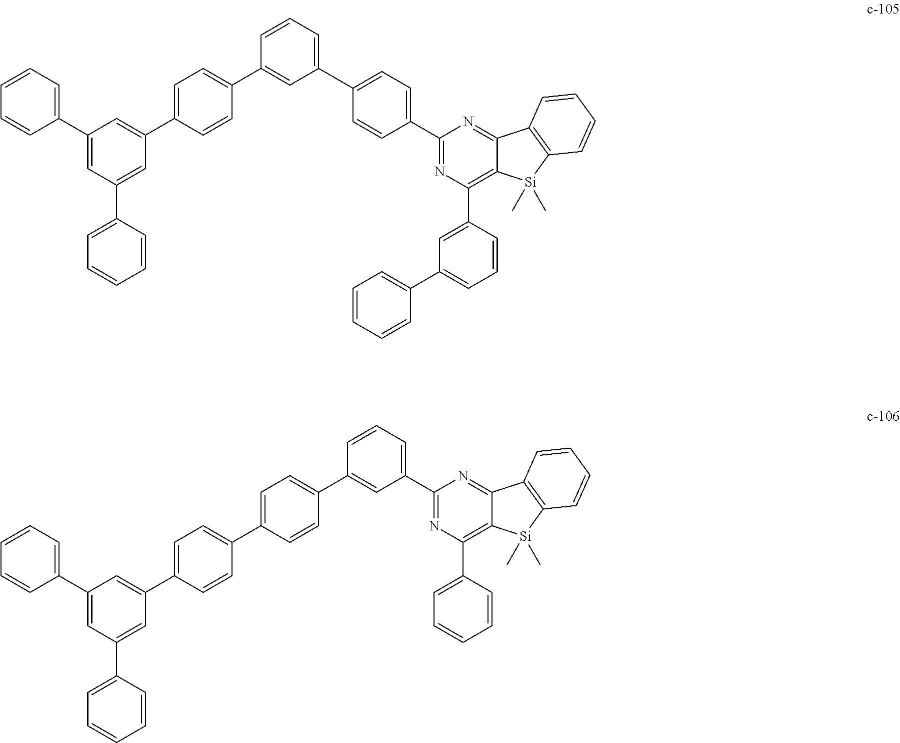 [SMILES: C[Si]1(C)c2ccccc2-c2nc(-c3ccc(-c4cccc(-c5ccc(-c6cc(-c7ccccc7)cc(-c7ccccc7)c6)cc5)c4)cc3)nc(-c3cccc(-c4ccccc4)c3)c21 ; C[Si]1(C)c2ccccc2-c2nc(-c3cccc(-c4ccc(-c5ccc(-c6cc(-c7ccccc7)cc(-c7ccccc7)c6)cc5)cc4)c3)nc(-c3ccccc3)c21]

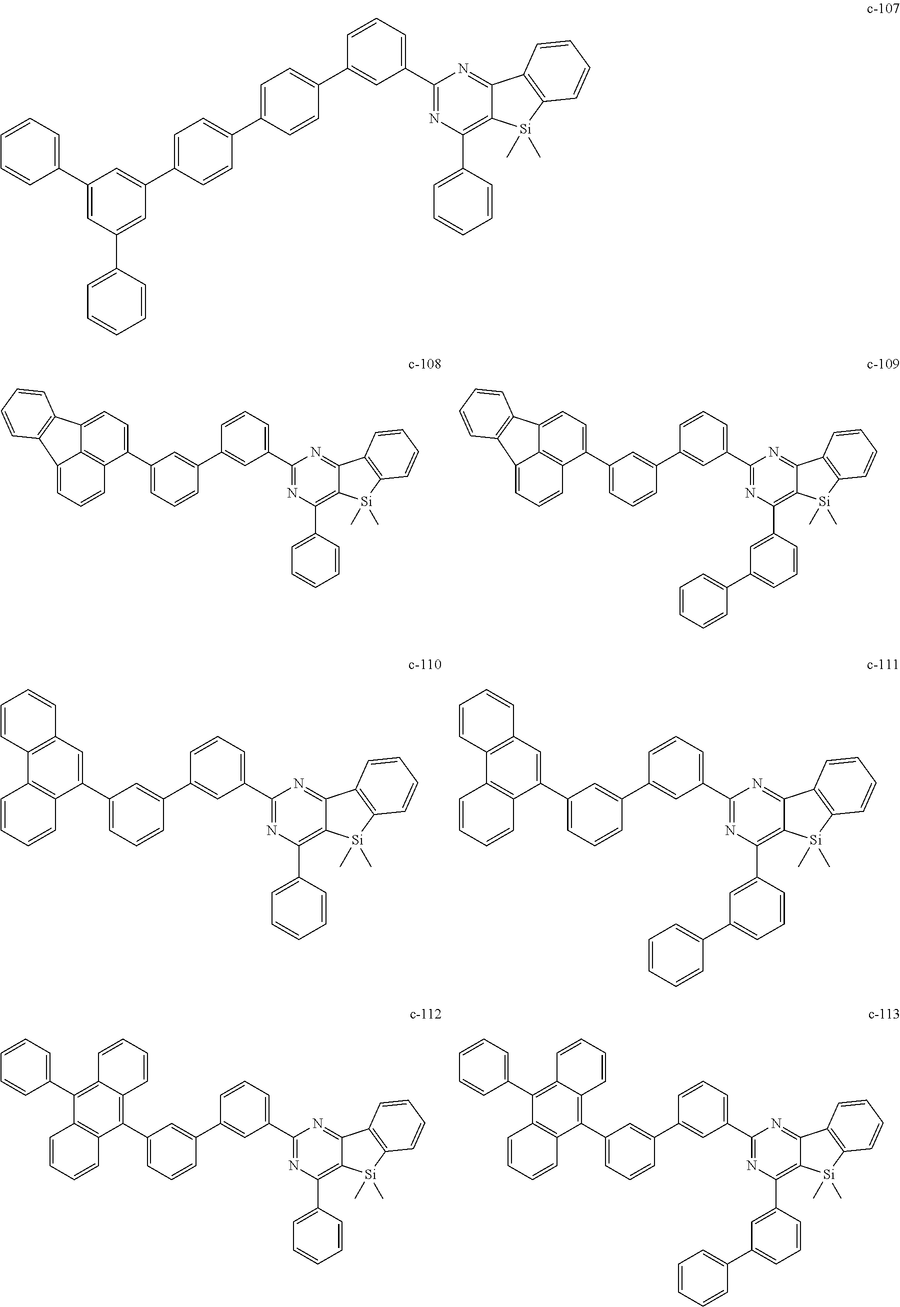

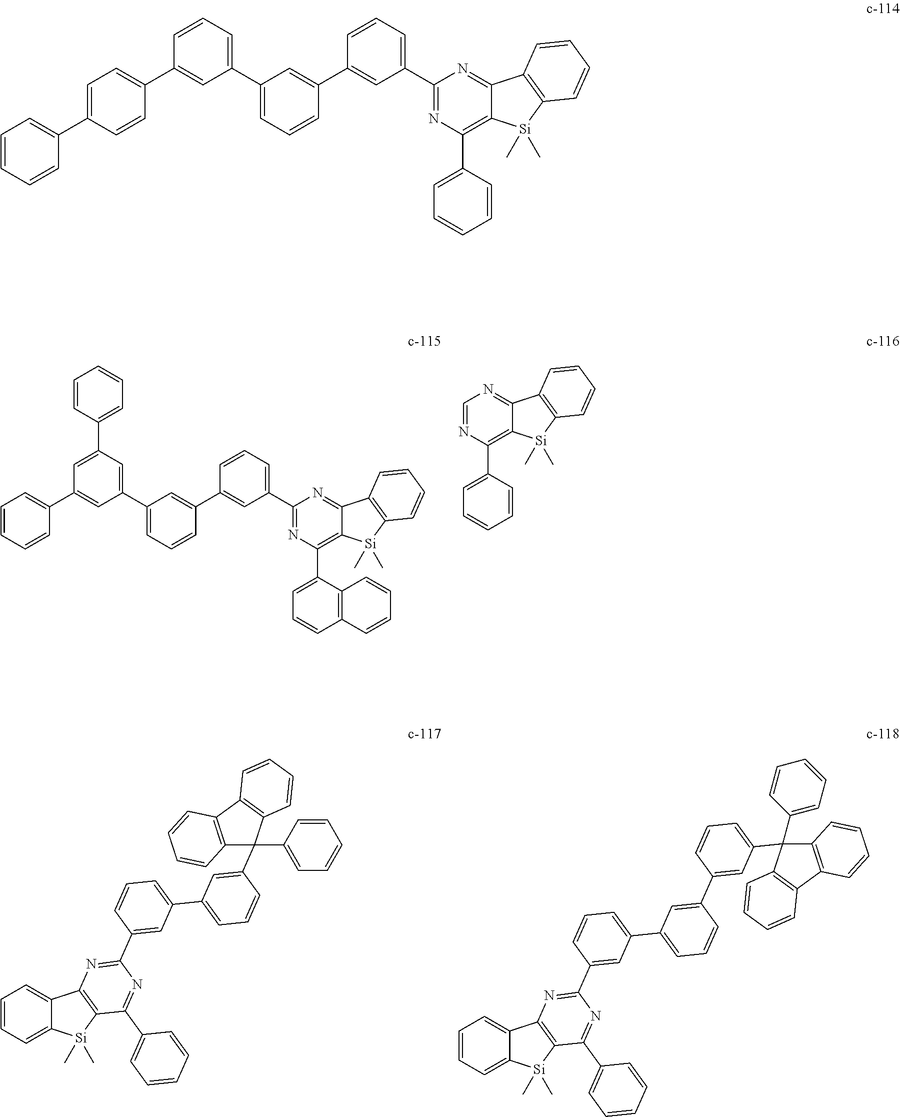

Group of X₁=N-[(L₁)ₐ₁-(R₁)_{b1}] in Formula 1-1

(L₁, a1, R₁ and b1 are described in the present specification)

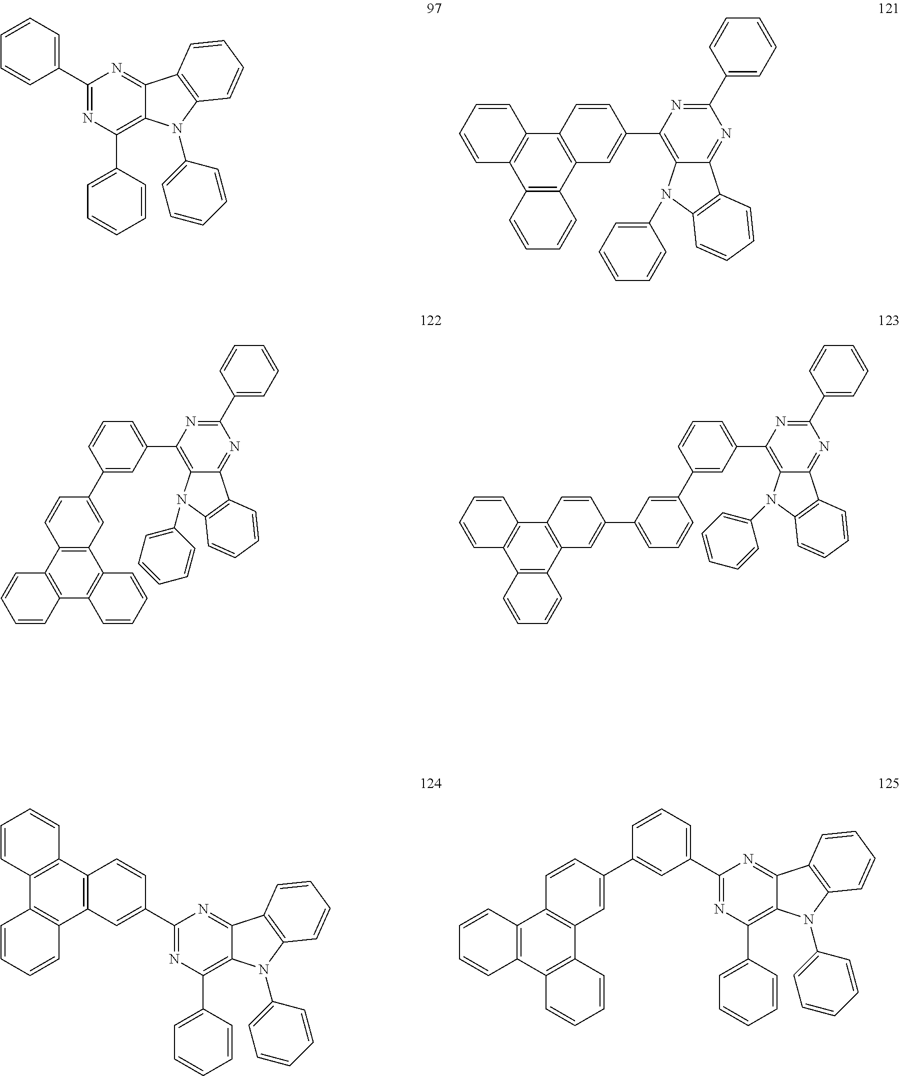 [SMILES: c1ccc(-c2nc(-c3ccccc3)c3c(n2)c2ccccc2n3-c2ccccc2)cc1 ; c1ccc(-c2nc(-c3ccc4c5ccccc5c5ccccc5c4c3)c3c(n2)c2ccccc2n3-c2ccccc2)cc1 ; c1ccc(-c2nc(-c3cccc(-c4ccc5c6ccccc6c6ccccc6c5c4)c3)c3c(n2)c2ccccc2n3-c2ccccc2)cc1 ; c1ccc(-c2nc(-c3cccc(-c4cccc(-c5ccc6c7ccccc7c7ccccc7c6c5)c4)c3)c3c(n2)c2ccccc2n3-c2ccccc2)cc1 ; c1ccc(-c2nc(-c3ccc4c5ccccc5c5ccccc5c4c3)nc3c4ccccc4n(-c4ccccc4)c23)cc1 ; c1ccc(-c2nc(-c3cccc(-c4ccc5c6ccccc6c6ccccc6c5c4)c3)nc3c4ccccc4n(-c4ccccc4)c23)cc1]

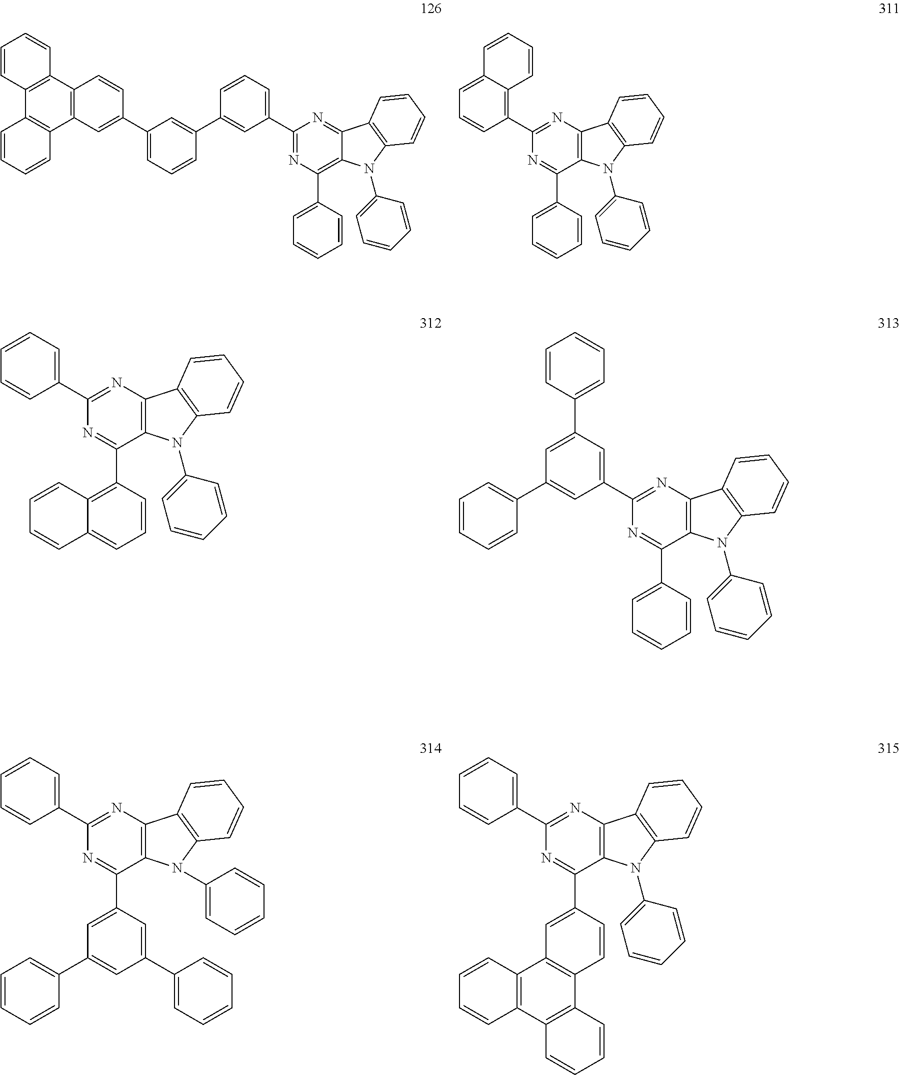

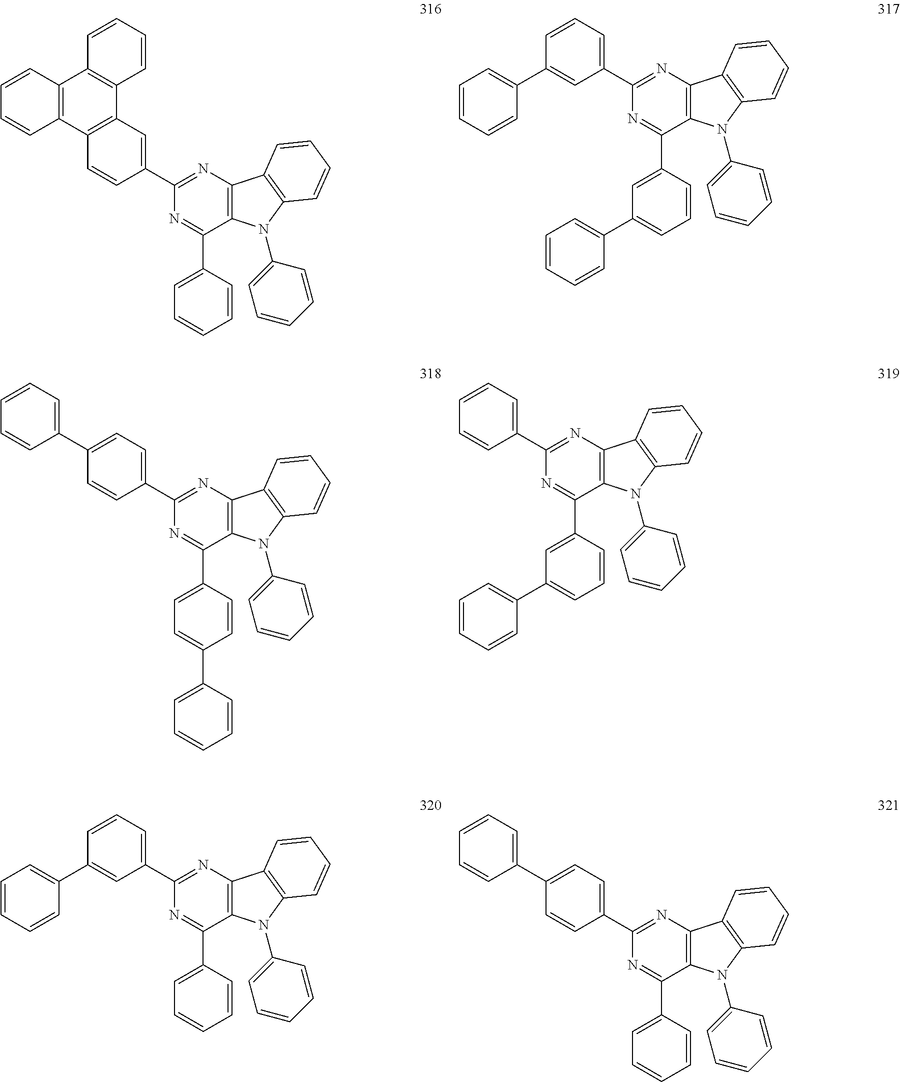 [SMILES: c1ccc(-c2nc(-c3ccc4c5ccccc5c5ccccc5c4c3)nc3c4ccccc4n(-c4ccccc4)c23)cc1 ; c1ccc(-c2cccc(-c3nc(-c4cccc(-c5ccccc5)c4)c4c(n3)c3ccccc3n4-c3ccccc3)c2)cc1 ; c1ccc(-c2ccc(-c3nc(-c4ccc(-c5ccccc5)cc4)c4c(n3)c3ccccc3n4-c3ccccc3)cc2)cc1 ; c1ccc(-c2cccc(-c3nc(-c4ccccc4)nc4c5ccccc5n(-c5ccccc5)c34)c2)cc1 ; c1ccc(-c2cccc(-c3nc(-c4ccccc4)c4c(n3)c3ccccc3n4-c3ccccc3)c2)cc1 ; c1ccc(-c2ccc(-c3nc(-c4ccccc4)c4c(n3)c3ccccc3n4-c3ccccc3)cc2)cc1]

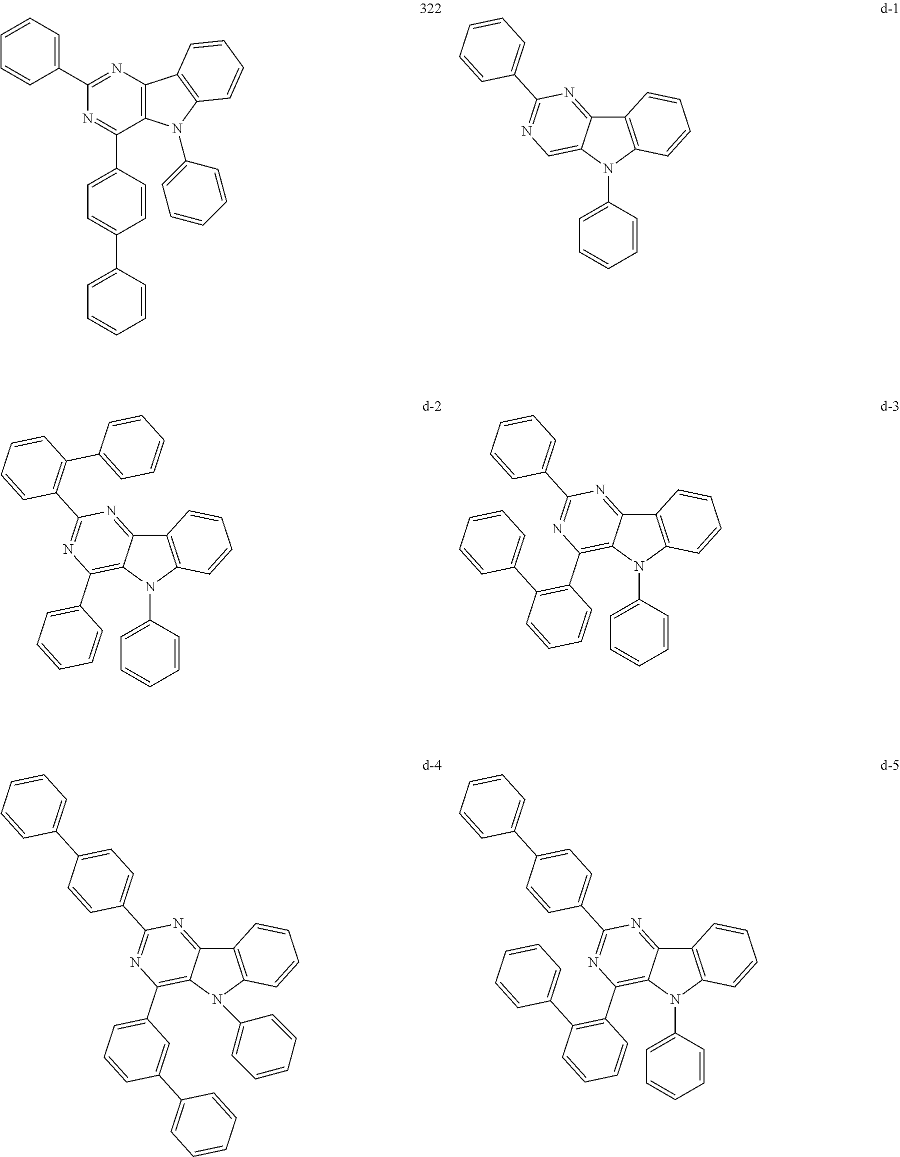 [SMILES: c1ccc(-c2ccc(-c3nc(-c4ccccc4)nc4c5ccccc5n(-c5ccccc5)c34)cc2)cc1 ; c1ccc(-c2ncc3c(n2)c2ccccc2n3-c2ccccc2)cc1 ; c1ccc(-c2ccccc2-c2nc(-c3ccccc3)c3c(n2)c2ccccc2n3-c2ccccc2)cc1 ; c1ccc(-c2nc(-c3ccccc3-c3ccccc3)c3c(n2)c2ccccc2n3-c2ccccc2)cc1 ; c1ccc(-c2ccc(-c3nc(-c4cccc(-c5ccccc5)c4)c4c(n3)c3ccccc3n4-c3ccccc3)cc2)cc1 ; c1ccc(-c2ccc(-c3nc(-c4ccccc4-c4ccccc4)c4c(n3)c3ccccc3n4-c3ccccc3)cc2)cc1]

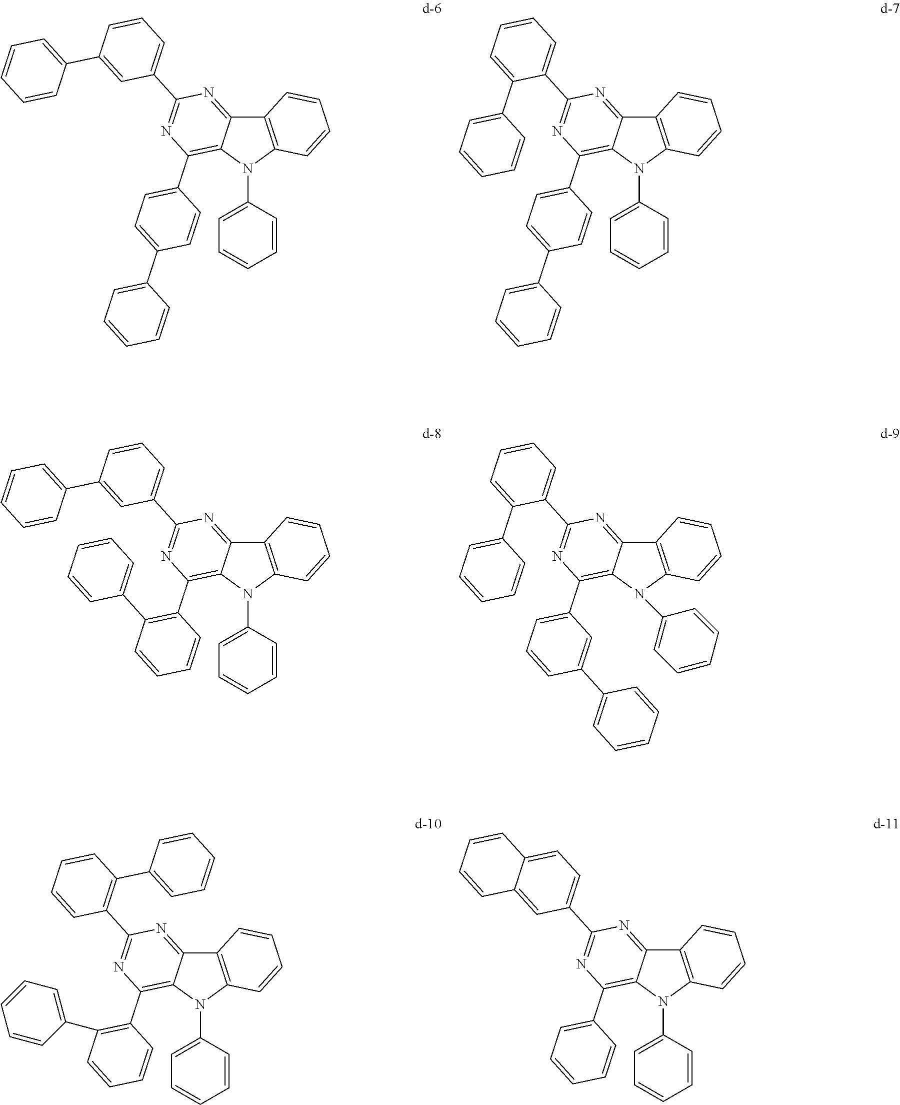 [SMILES: c1ccc(-c2ccc(-c3nc(-c4cccc(-c5ccccc5)c4)nc4c5ccccc5n(-c5ccccc5)c34)cc2)cc1 ; c1ccc(-c2ccc(-c3nc(-c4ccccc4-c4ccccc4)nc4c5ccccc5n(-c5ccccc5)c34)cc2)cc1 ; c1ccc(-c2cccc(-c3nc(-c4ccccc4-c4ccccc4)c4c(n3)c3ccccc3n4-c3ccccc3)c2)cc1 ; c1ccc(-c2cccc(-c3nc(-c4ccccc4-c4ccccc4)nc4c5ccccc5n(-c5ccccc5)c34)c2)cc1 ; c1ccc(-c2ccccc2-c2nc(-c3ccccc3-c3ccccc3)c3c(n2)c2ccccc2n3-c2ccccc2)cc1 ; c1ccc(-c2nc(-c3ccc4ccccc4c3)nc3c4ccccc4n(-c4ccccc4)c23)cc1]

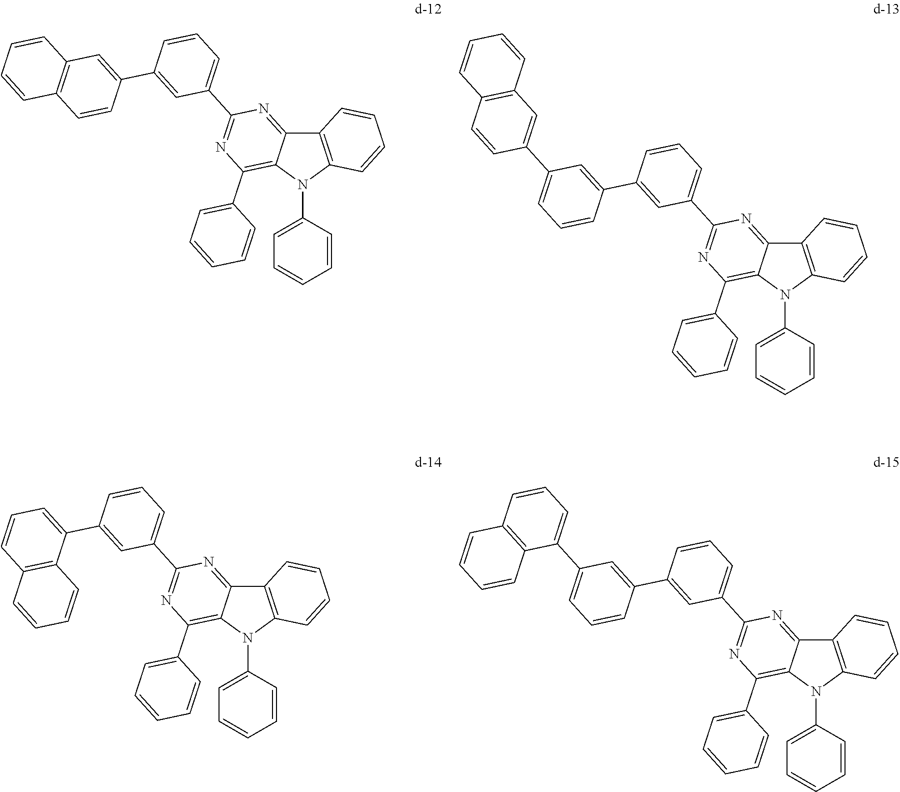 [SMILES: c1ccc(-c2nc(-c3cccc(-c4ccc5ccccc5c4)c3)nc3c4ccccc4n(-c4ccccc4)c23)cc1 ; c1ccc(-c2nc(-c3cccc(-c4cccc(-c5ccc6ccccc6c5)c4)c3)nc3c4ccccc4n(-c4ccccc4)c23)cc1 ; c1ccc(-c2nc(-c3cccc(-c4cccc5ccccc45)c3)nc3c4ccccc4n(-c4ccccc4)c23)cc1 ; c1ccc(-c2nc(-c3cccc(-c4cccc(-c5cccc6ccccc56)c4)c3)nc3c4ccccc4n(-c4ccccc4)c23)cc1]

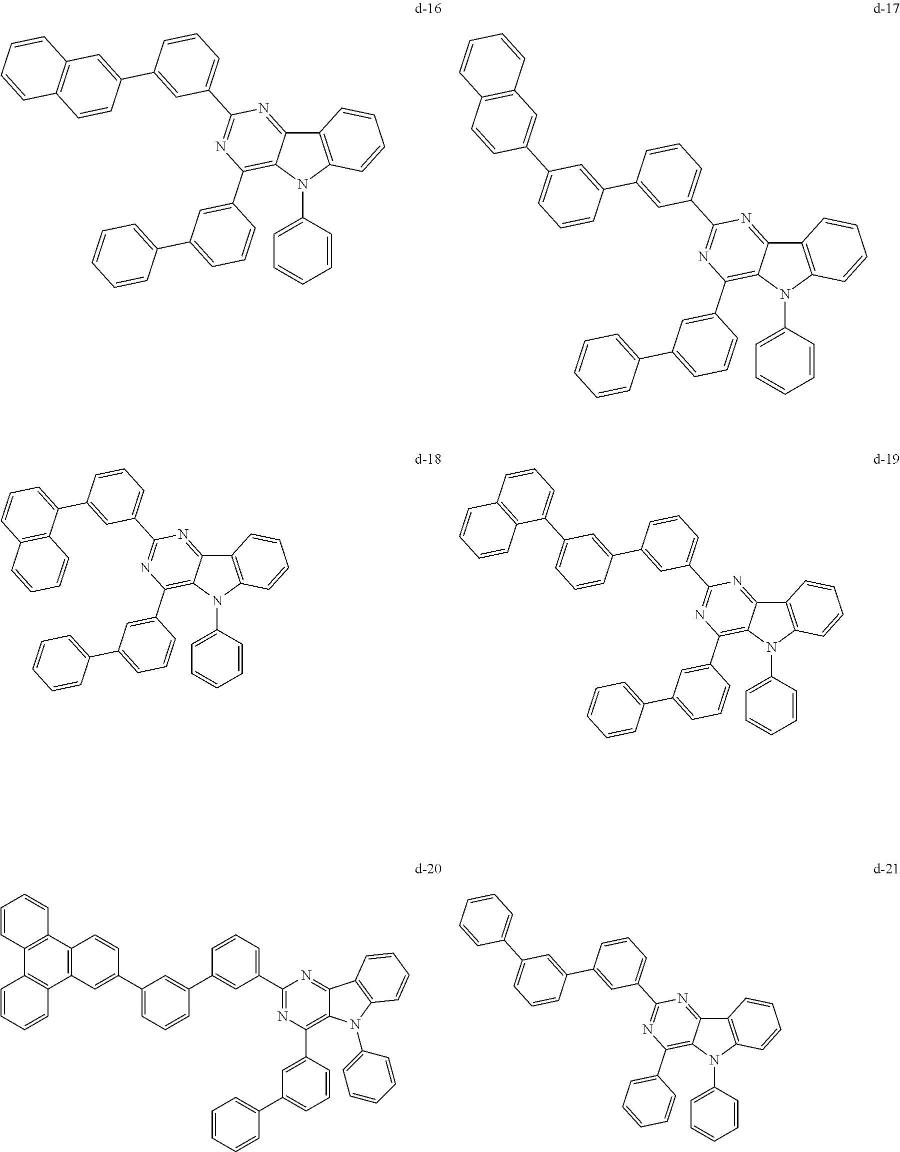

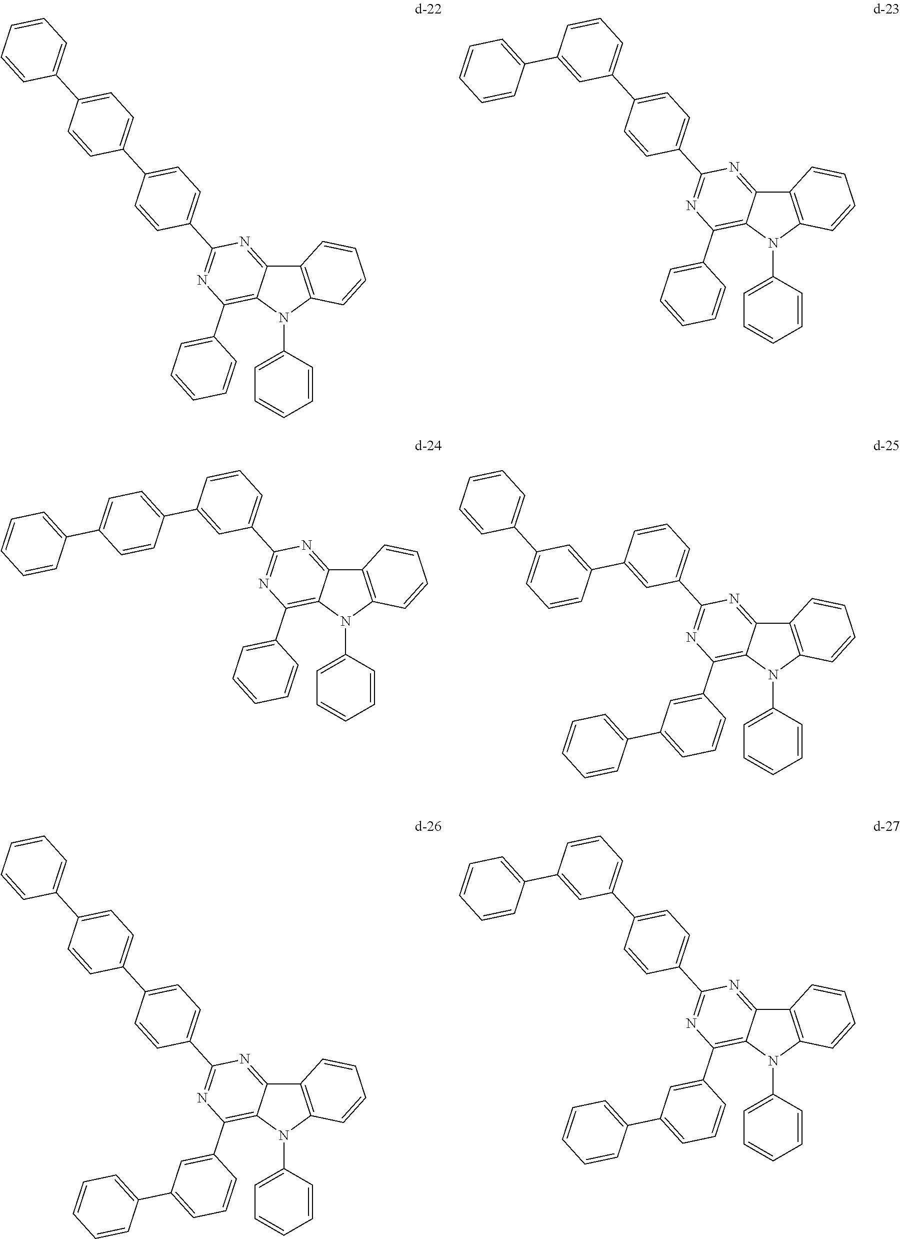 [SMILES: c1ccc(-c2ccc(-c3ccc(-c4nc(-c5ccccc5)c5c(n4)c4ccccc4n5-c4ccccc4)cc3)cc2)cc1 ; c1ccc(-c2cccc(-c3ccc(-c4nc(-c5ccccc5)c5c(n4)c4ccccc4n5-c4ccccc4)cc3)c2)cc1 ; c1ccc(-c2ccc(-c3cccc(-c4nc(-c5ccccc5)c5c(n4)c4ccccc4n5-c4ccccc4)c3)cc2)cc1 ; c1ccc(-c2cccc(-c3cccc(-c4nc(-c5cccc(-c6ccccc6)c5)c5c(n4)c4ccccc4n5-c4ccccc4)c3)c2)cc1 ; c1ccc(-c2ccc(-c3ccc(-c4nc(-c5cccc(-c6ccccc6)c5)c5c(n4)c4ccccc4n5-c4ccccc4)cc3)cc2)cc1 ; c1ccc(-c2cccc(-c3ccc(-c4nc(-c5cccc(-c6ccccc6)c5)c5c(n4)c4ccccc4n5-c4ccccc4)cc3)c2)cc1]

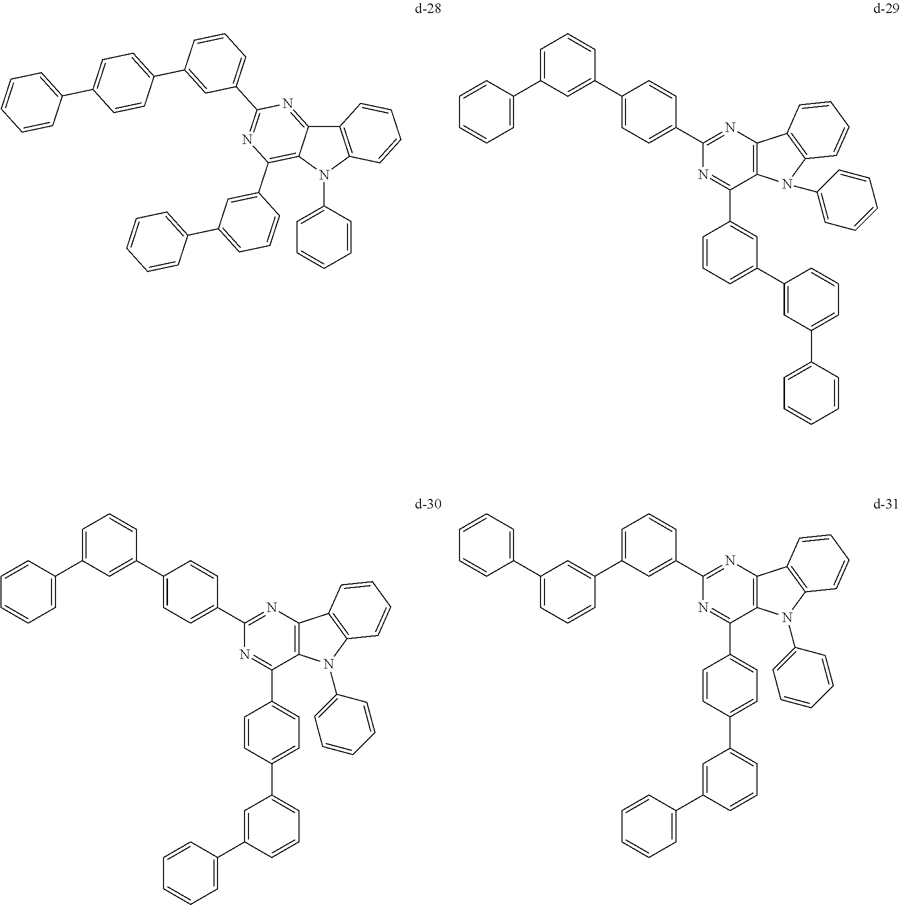 [SMILES: c1ccc(-c2ccc(-c3cccc(-c4nc(-c5cccc(-c6ccccc6)c5)c5c(n4)c4ccccc4n5-c4ccccc4)c3)cc2)cc1 ; c1ccc(-c2cccc(-c3ccc(-c4nc(-c5cccc(-c6cccc(-c7ccccc7)c6)c5)c5c(n4)c4ccccc4n5-c4ccccc4)cc3)c2)cc1 ; c1ccc(-c2cccc(-c3ccc(-c4nc(-c5ccc(-c6cccc(-c7ccccc7)c6)cc5)c5c(n4)c4ccccc4n5-c4ccccc4)cc3)c2)cc1 ; c1ccc(-c2cccc(-c3ccc(-c4nc(-c5cccc(-c6cccc(-c7ccccc7)c6)c5)nc5c6ccccc6n(-c6ccccc6)c45)cc3)c2)cc1]

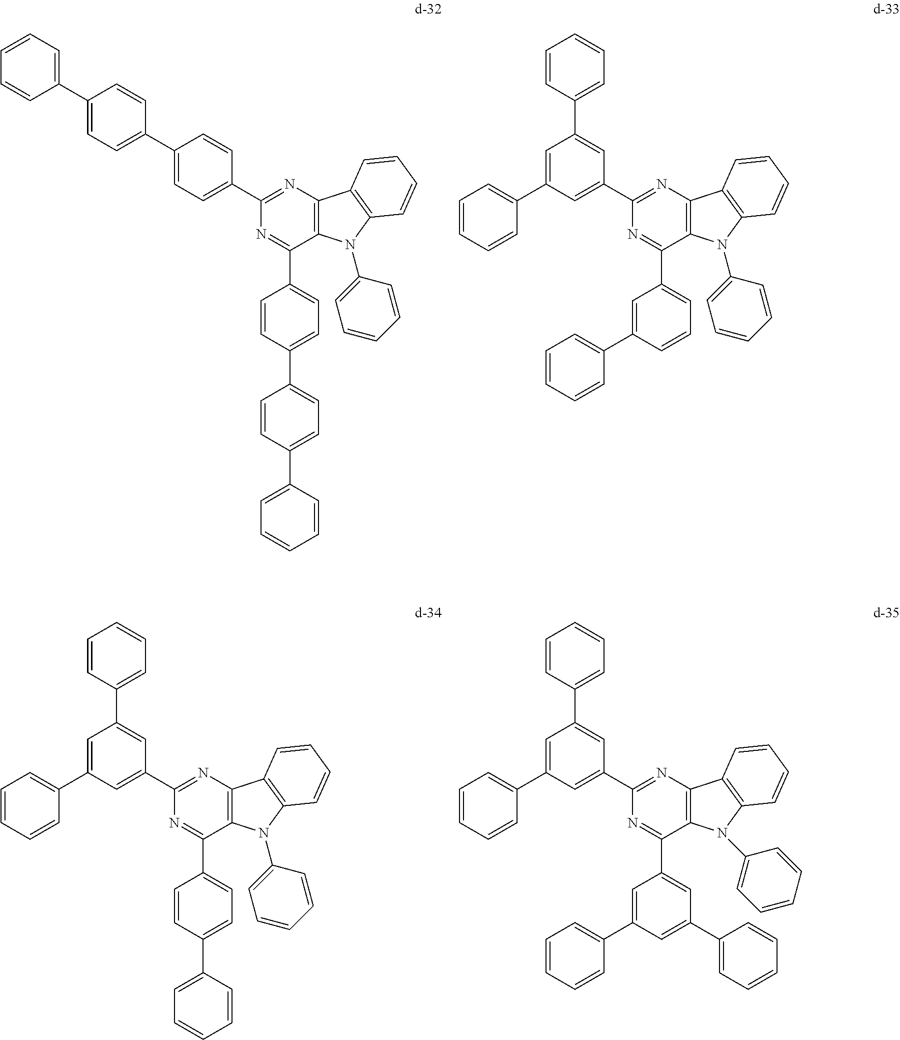

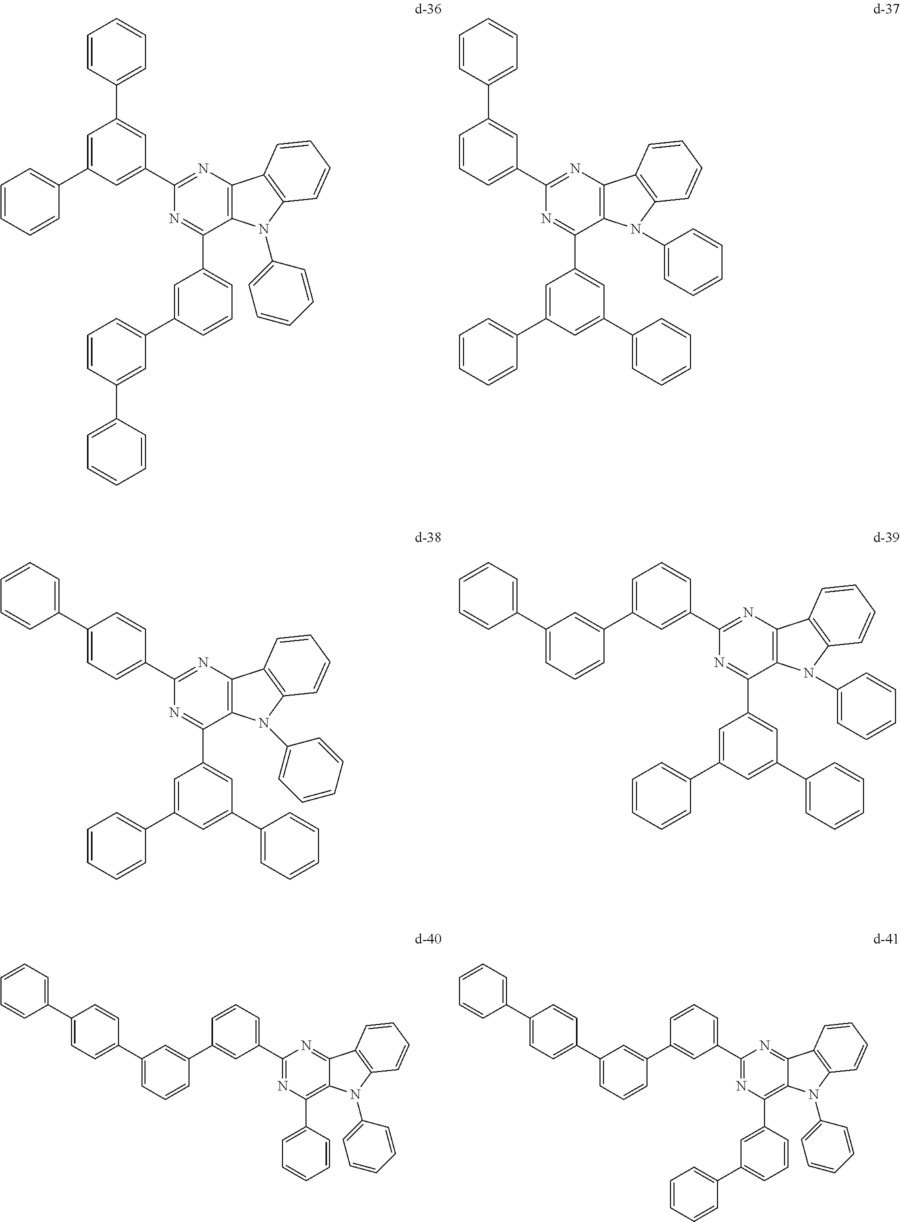

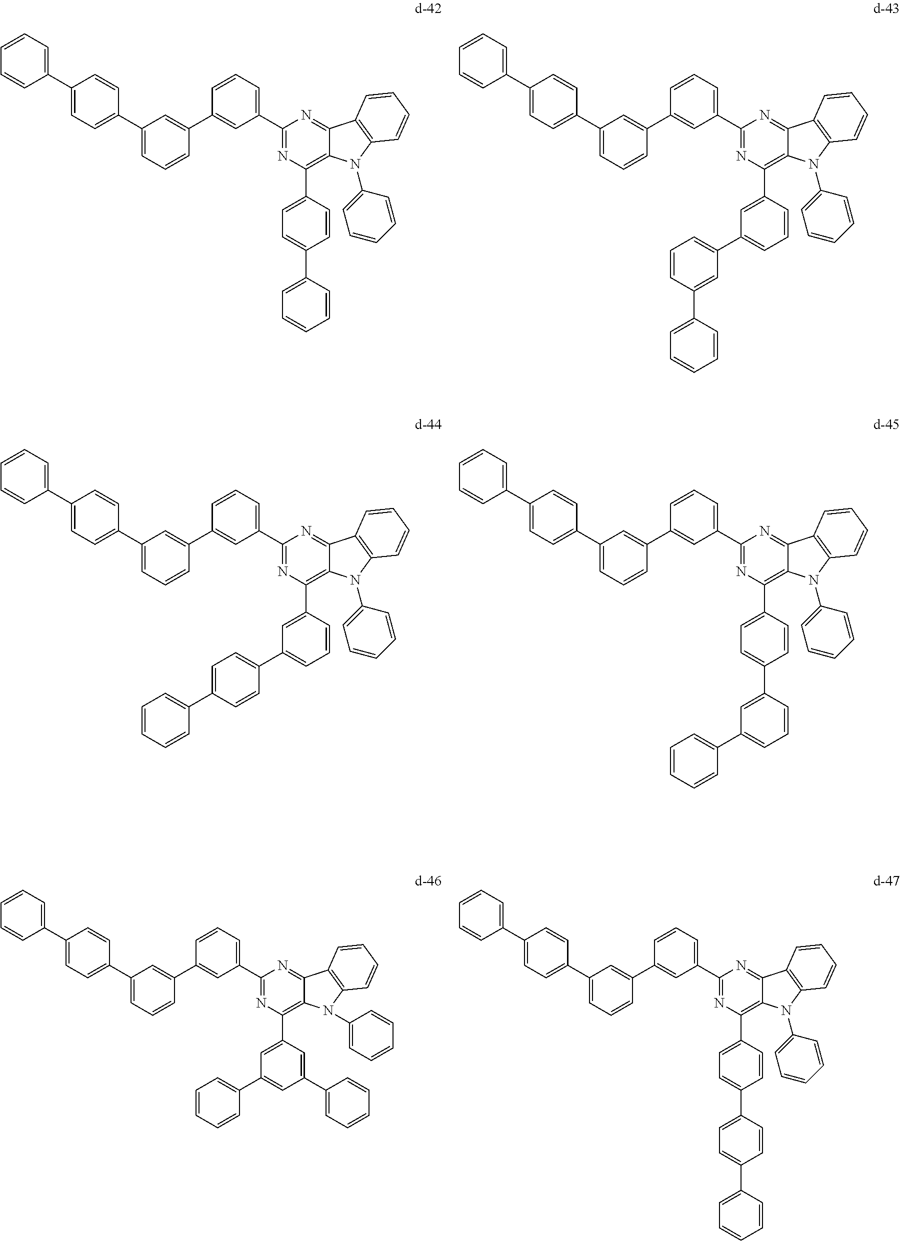

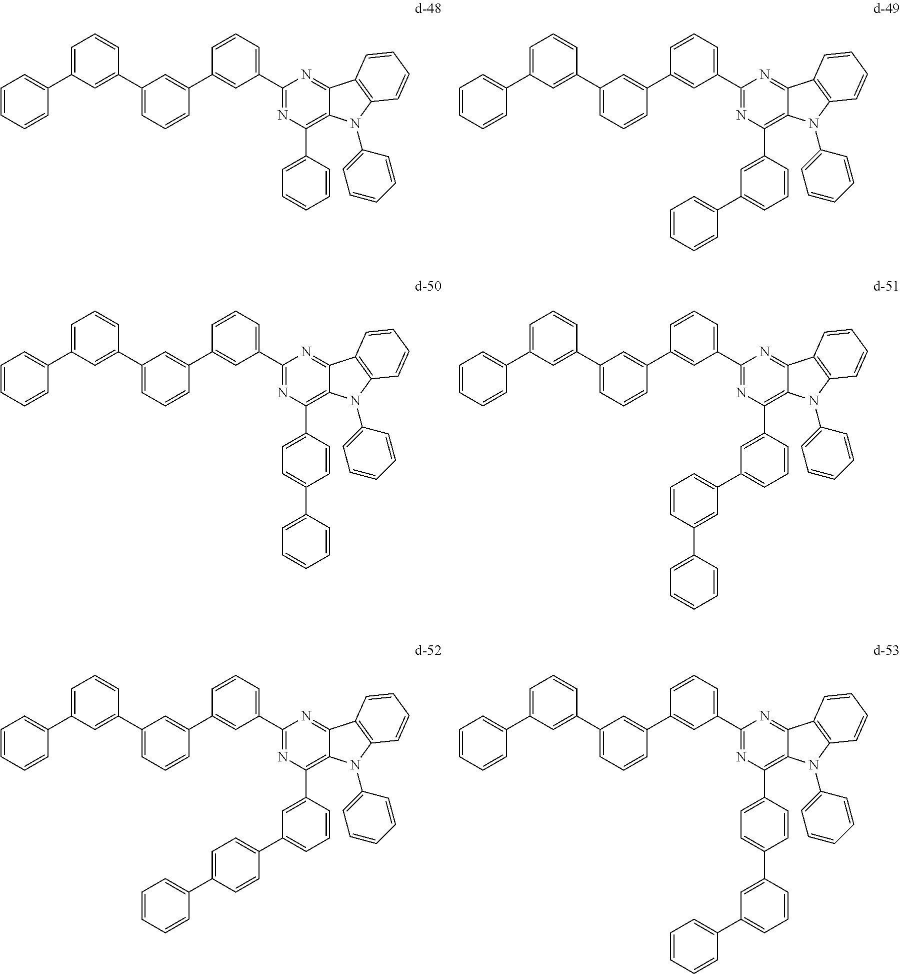

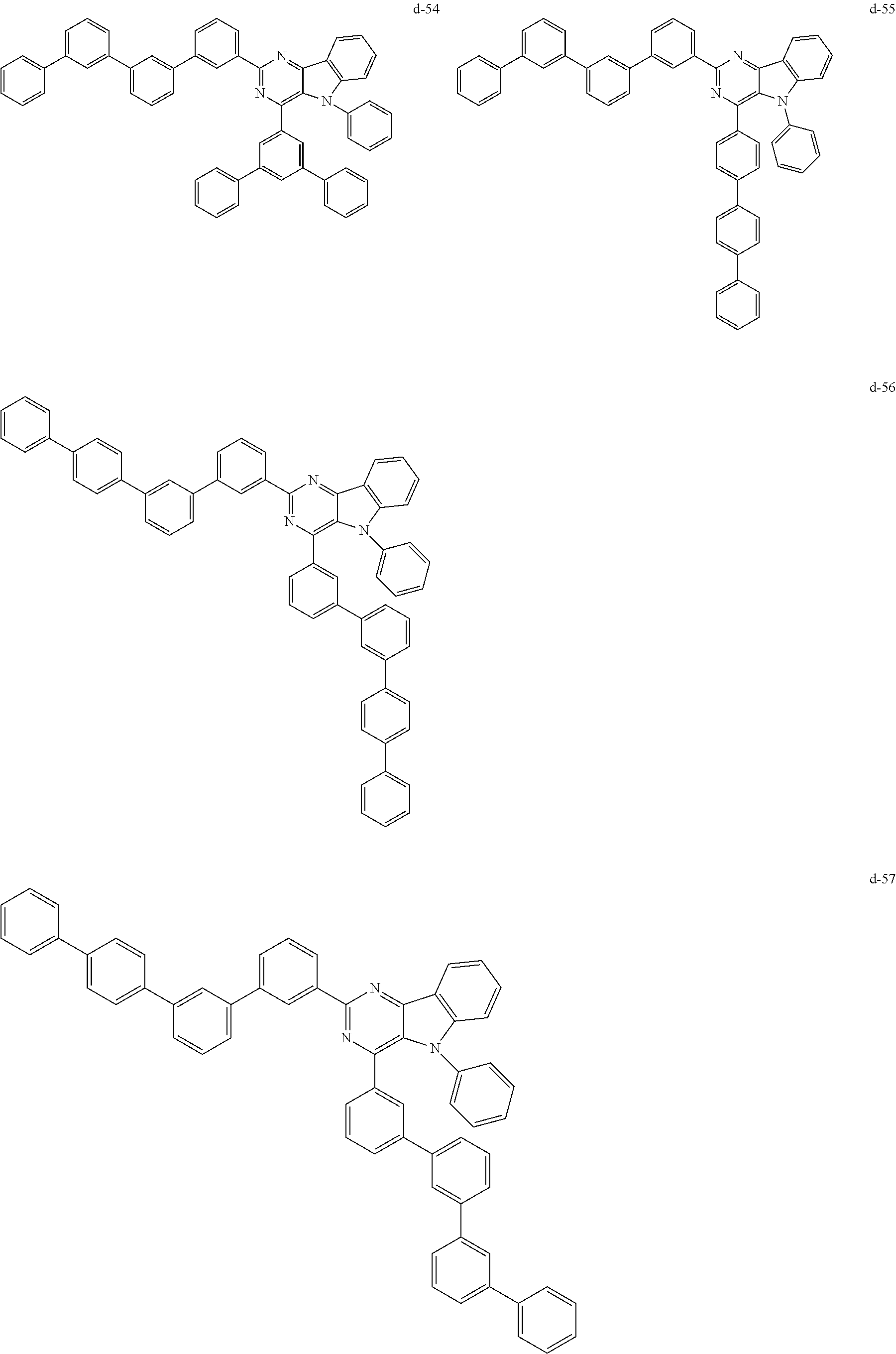 [SMILES: c1ccc(-c2cccc(-c3cccc(-c4cccc(-c5nc(-c6cc(-c7ccccc7)cc(-c7ccccc7)c6)c6c(n5)c5ccccc5n6-c5ccccc5)c4)c3)c2)cc1 ; c1ccc(-c2ccc(-c3ccc(-c4nc(-c5cccc(-c6cccc(-c7cccc(-c8ccccc8)c7)c6)c5)nc5c6ccccc6n(-c6ccccc6)c45)cc3)cc2)cc1 ; c1ccc(-c2ccc(-c3cccc(-c4cccc(-c5nc(-c6cccc(-c7cccc(-c8ccc(-c9ccccc9)cc8)c7)c6)c6c(n5)c5ccccc5n6-c5ccccc5)c4)c3)cc2)cc1 ; c1ccc(-c2ccc(-c3cccc(-c4cccc(-c5nc(-c6cccc(-c7cccc(-c8cccc(-c9ccccc9)c8)c7)c6)c6c(n5)c5ccccc5n6-c5ccccc5)c4)c3)cc2)cc1]

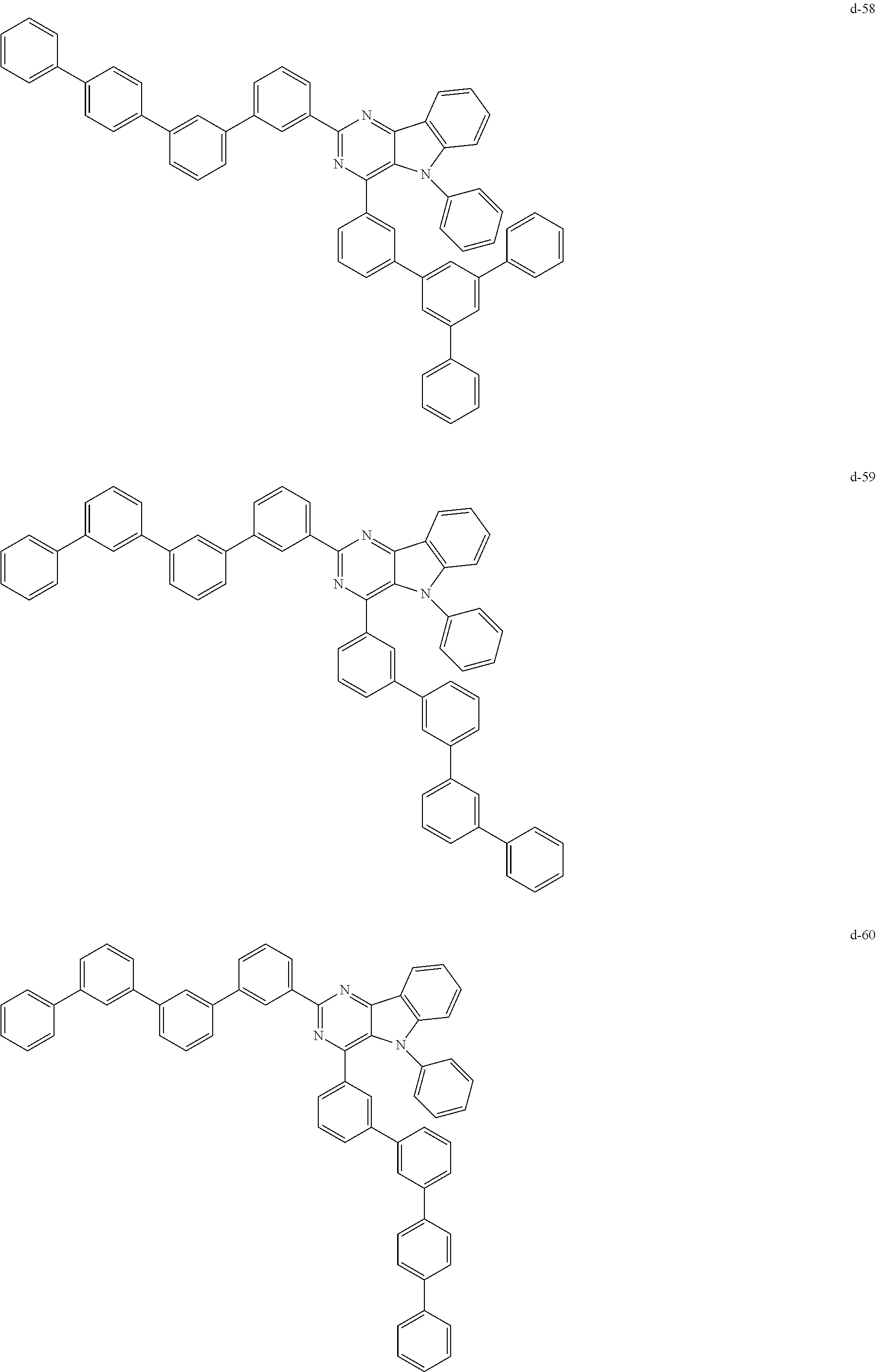 [SMILES: c1ccc(-c2ccc(-c3cccc(-c4cccc(-c5nc(-c6cccc(-c7cc(-c8ccccc8)cc(-c8ccccc8)c7)c6)c6c(n5)c5ccccc5n6-c5ccccc5)c4)c3)cc2)cc1 ; c1ccc(-c2cccc(-c3cccc(-c4cccc(-c5nc(-c6cccc(-c7cccc(-c8cccc(-c9ccccc9)c8)c7)c6)c6c(n5)c5ccccc5n6-c5ccccc5)c4)c3)c2)cc1 ; c1ccc(-c2ccc(-c3cccc(-c4cccc(-c5nc(-c6cccc(-c7cccc(-c8cccc(-c9ccccc9)c8)c7)c6)nc6c7ccccc7n(-c7ccccc7)c56)c4)c3)cc2)cc1]

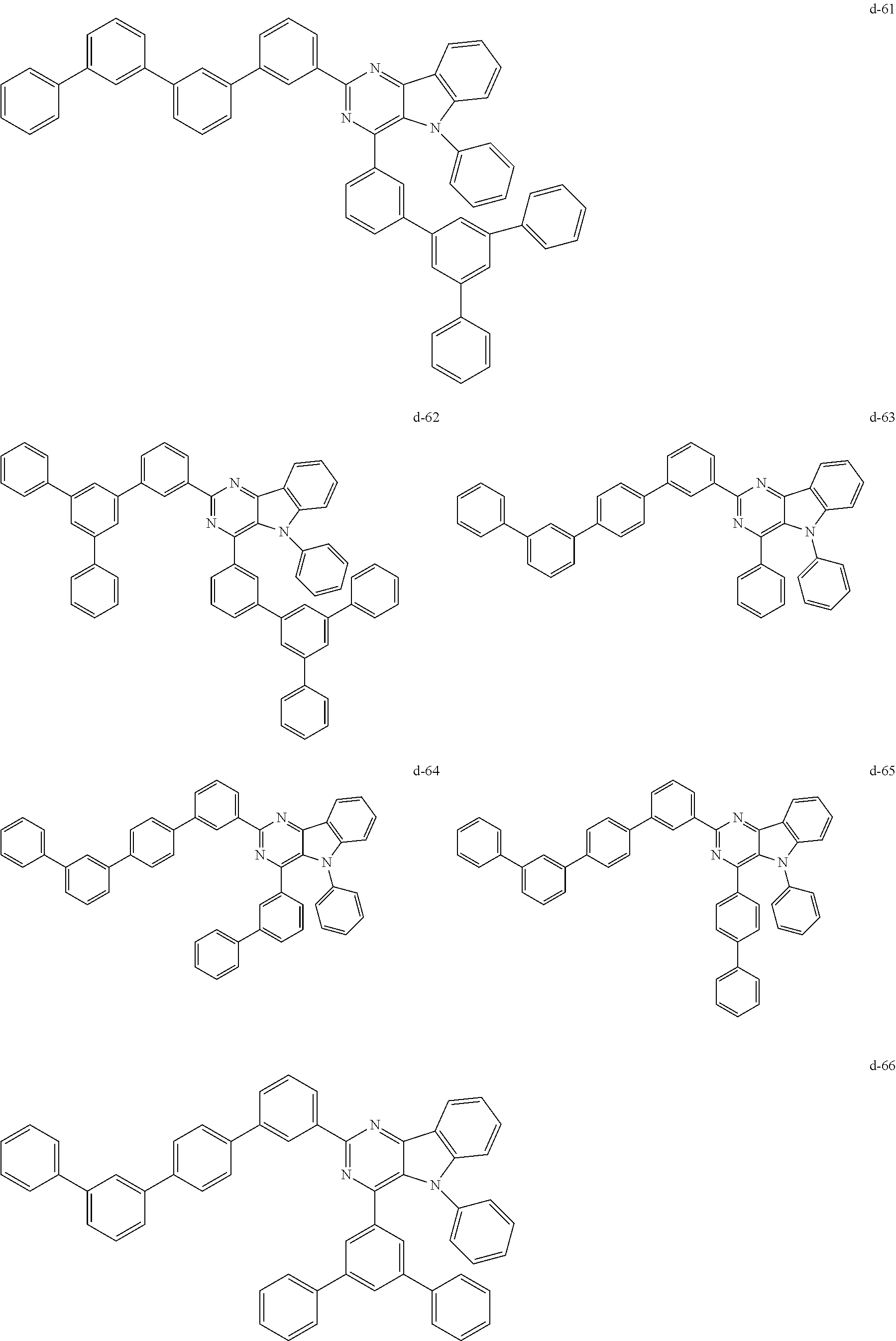

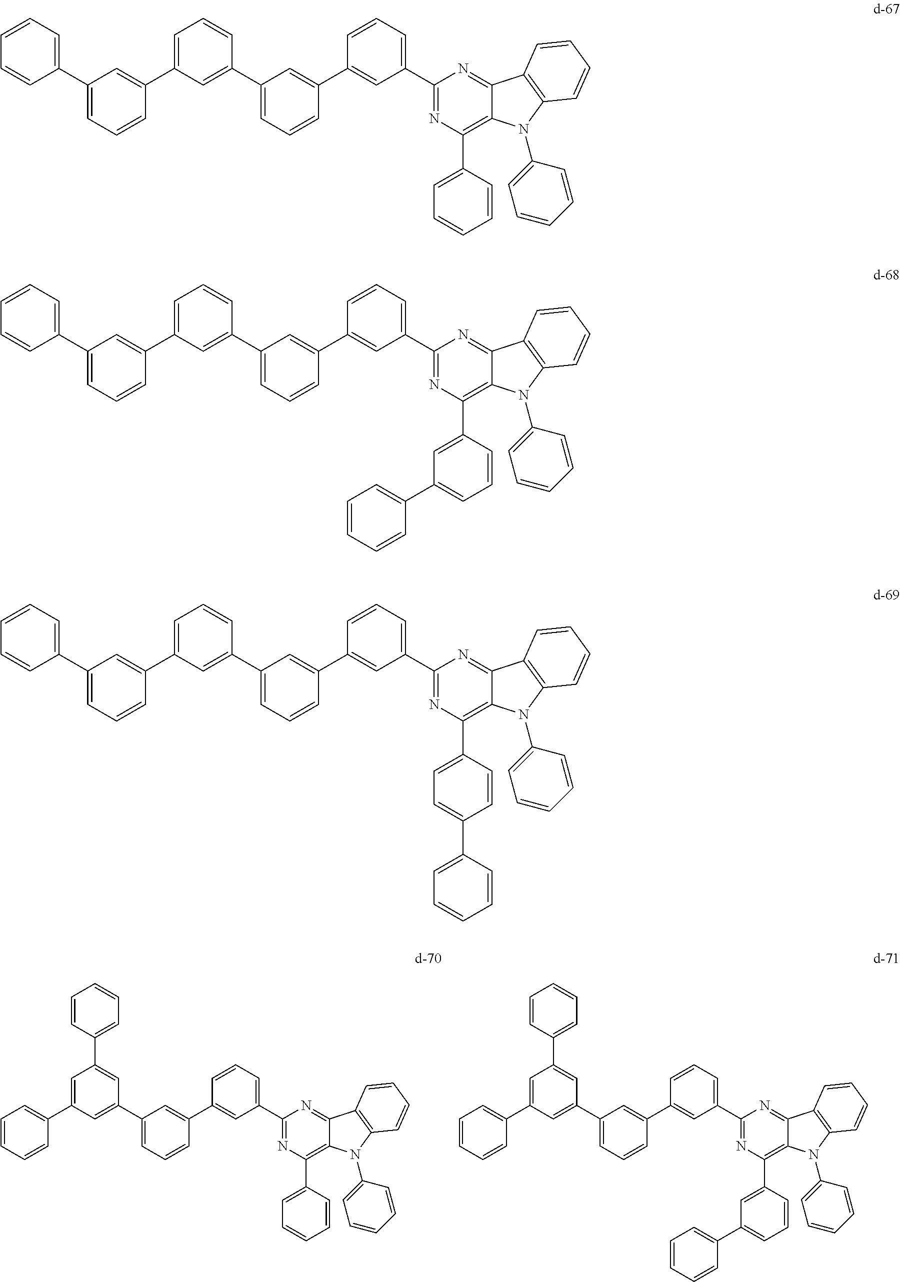

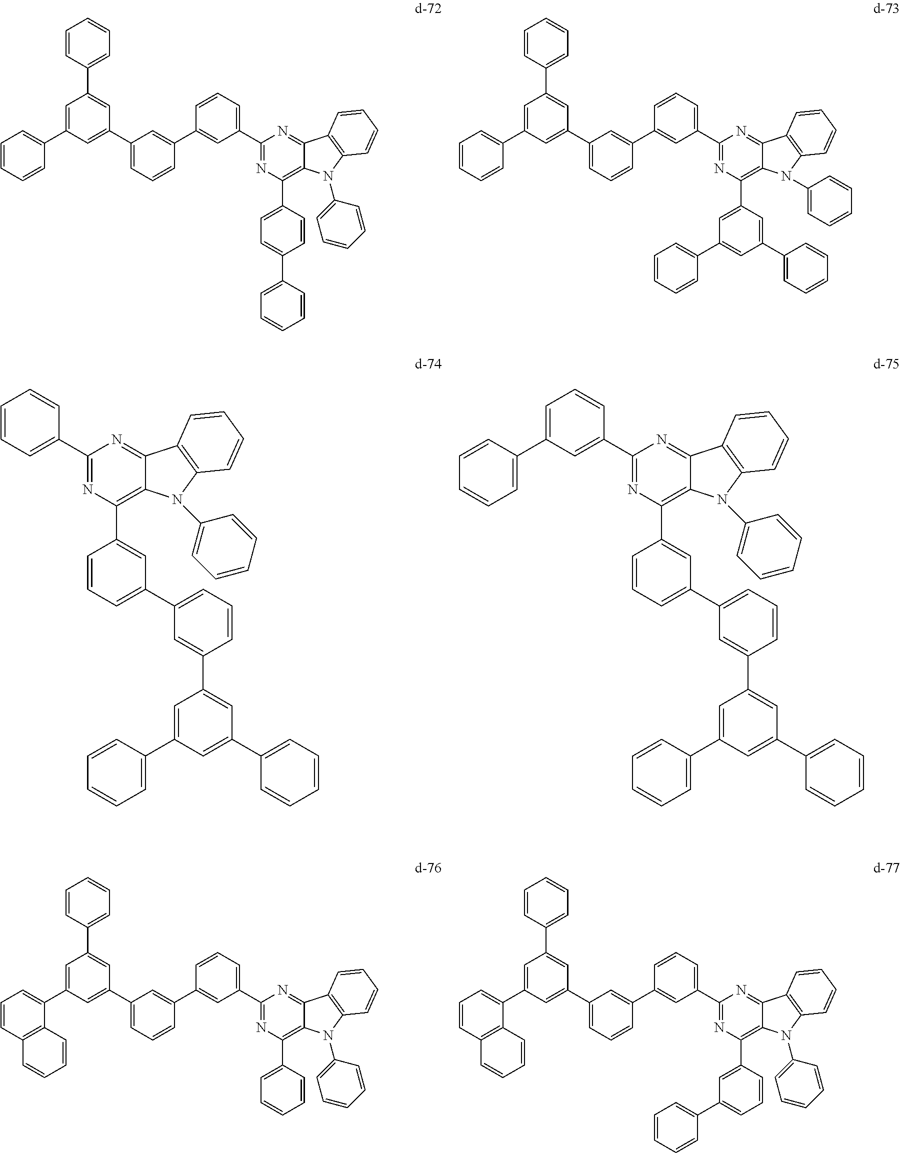

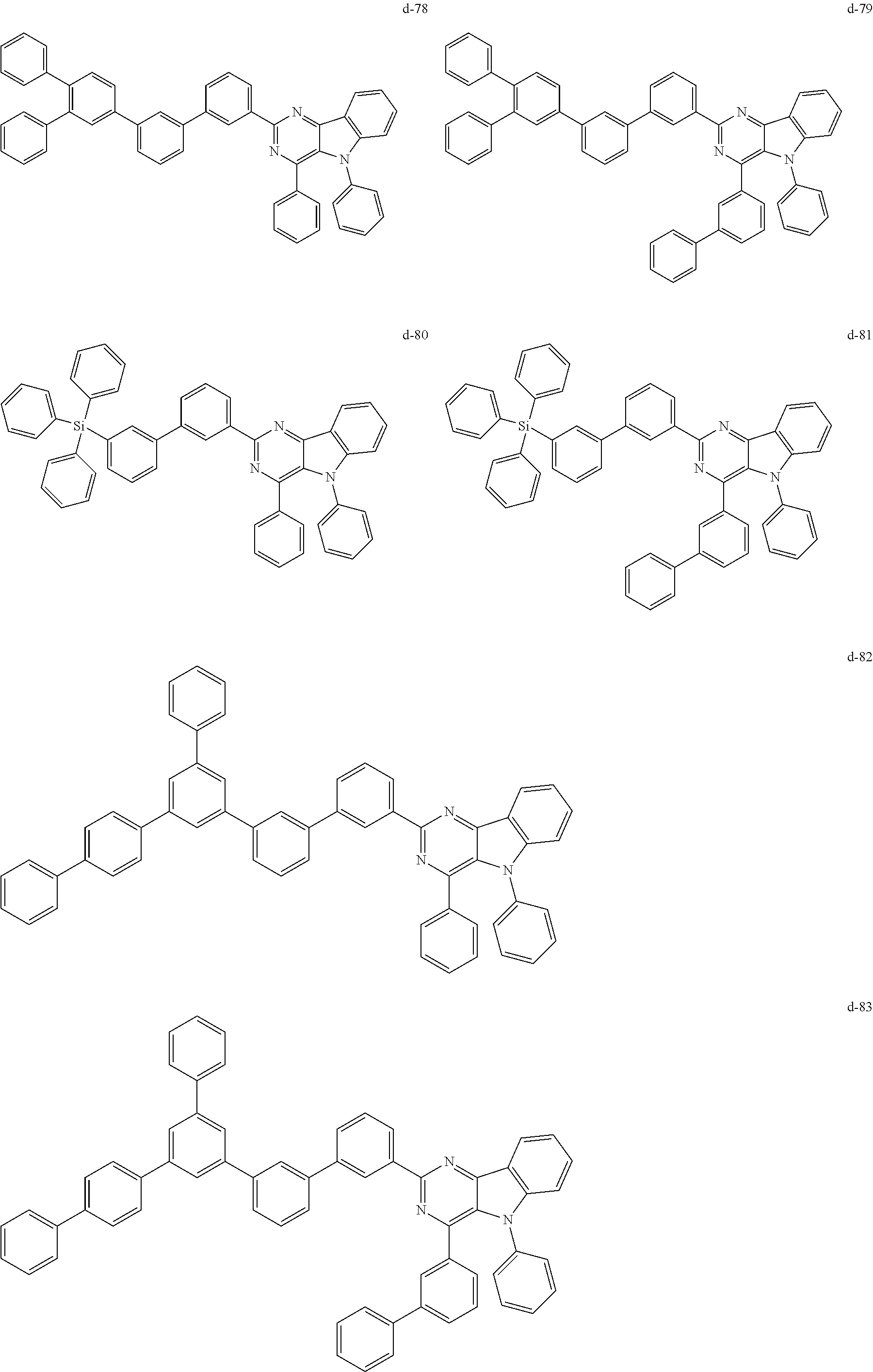

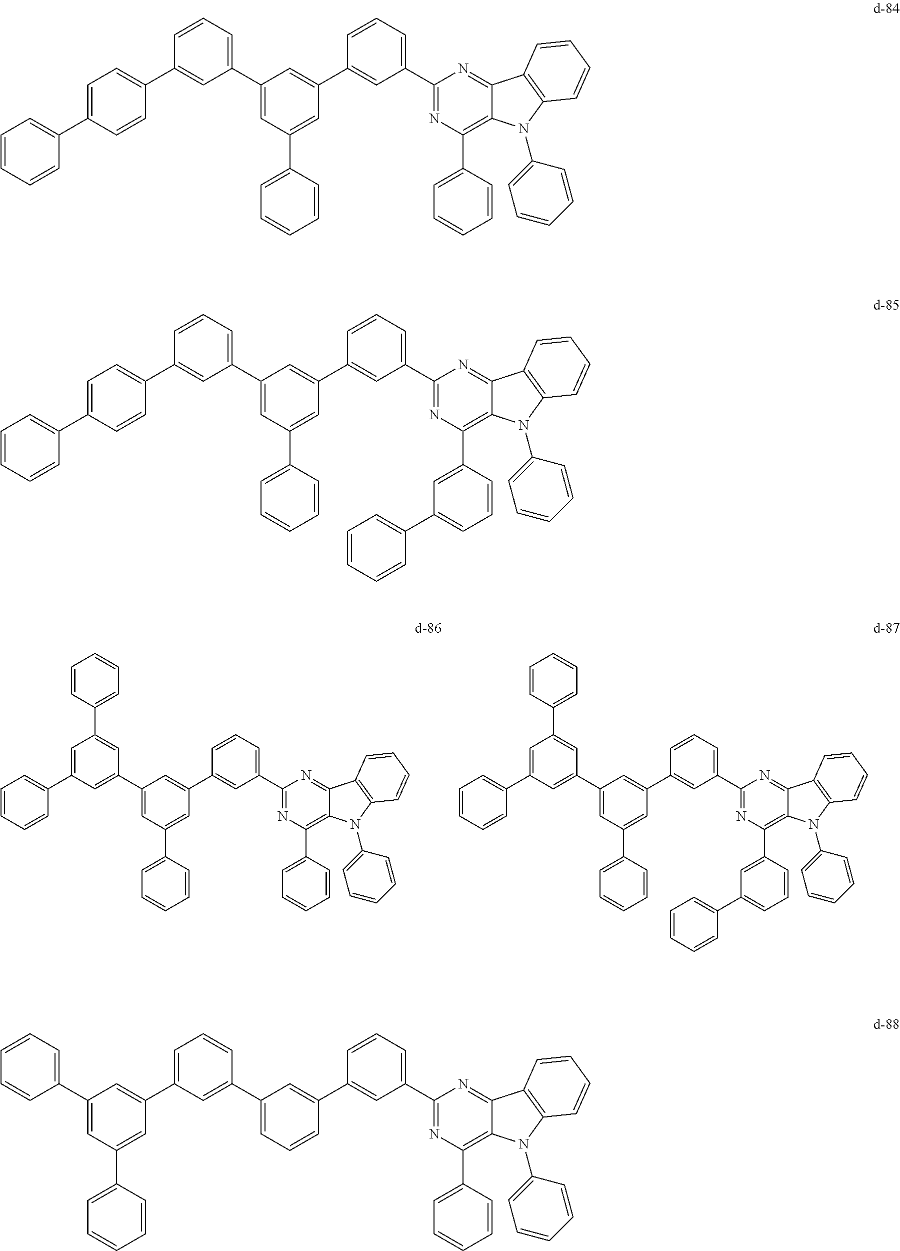

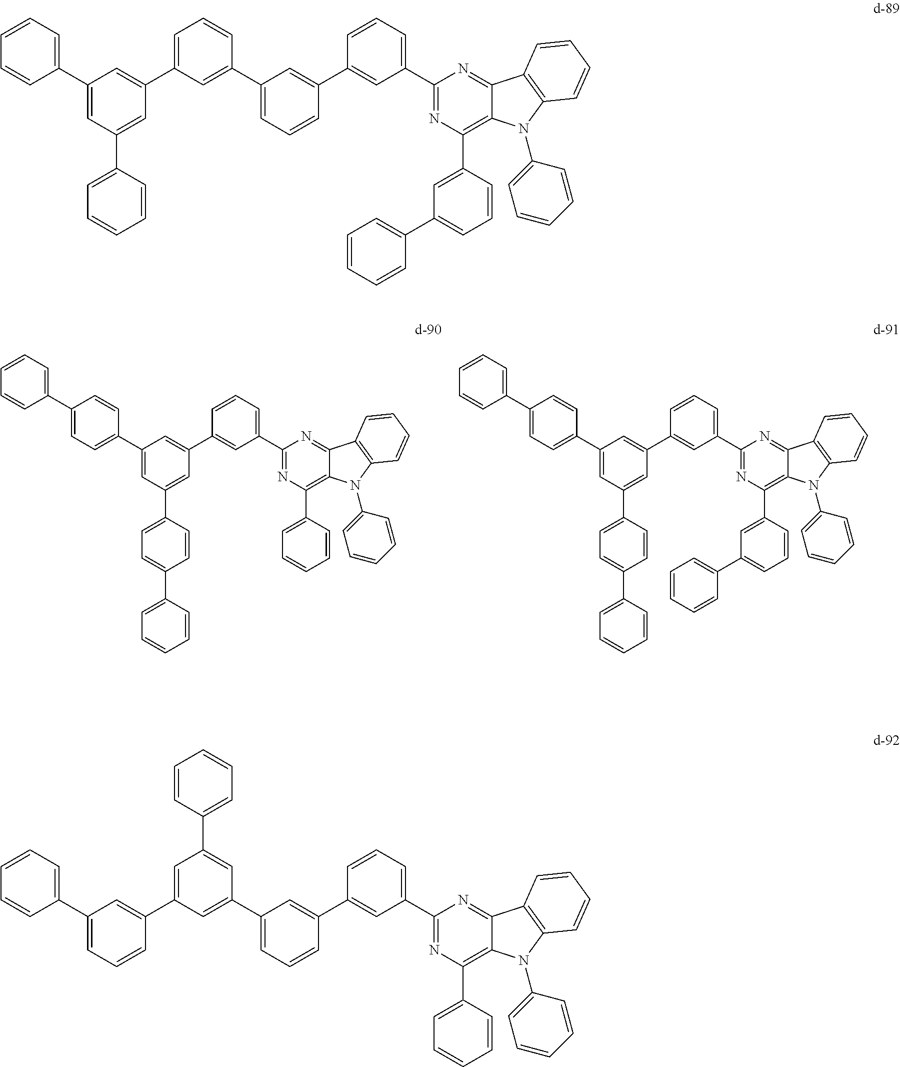 [SMILES: c1ccc(-c2cc(-c3ccccc3)cc(-c3cccc(-c4cccc(-c5cccc(-c6nc(-c7cccc(-c8ccccc8)c7)c7c(n6)c6ccccc6n7-c6ccccc6)c5)c4)c3)c2)cc1 ; c1ccc(-c2ccc(-c3cc(-c4ccc(-c5ccccc5)cc4)cc(-c4cccc(-c5nc(-c6ccccc6)c6c(n5)c5ccccc5n6-c5ccccc5)c4)c3)cc2)cc1 ; c1ccc(-c2ccc(-c3cc(-c4ccc(-c5ccccc5)cc4)cc(-c4cccc(-c5nc(-c6cccc(-c7ccccc7)c6)c6c(n5)c5ccccc5n6-c5ccccc5)c4)c3)cc2)cc1 ; c1ccc(-c2cccc(-c3cc(-c4ccccc4)cc(-c4cccc(-c5cccc(-c6nc(-c7ccccc7)c7c(n6)c6ccccc6n7-c6ccccc6)c5)c4)c3)c2)cc1]

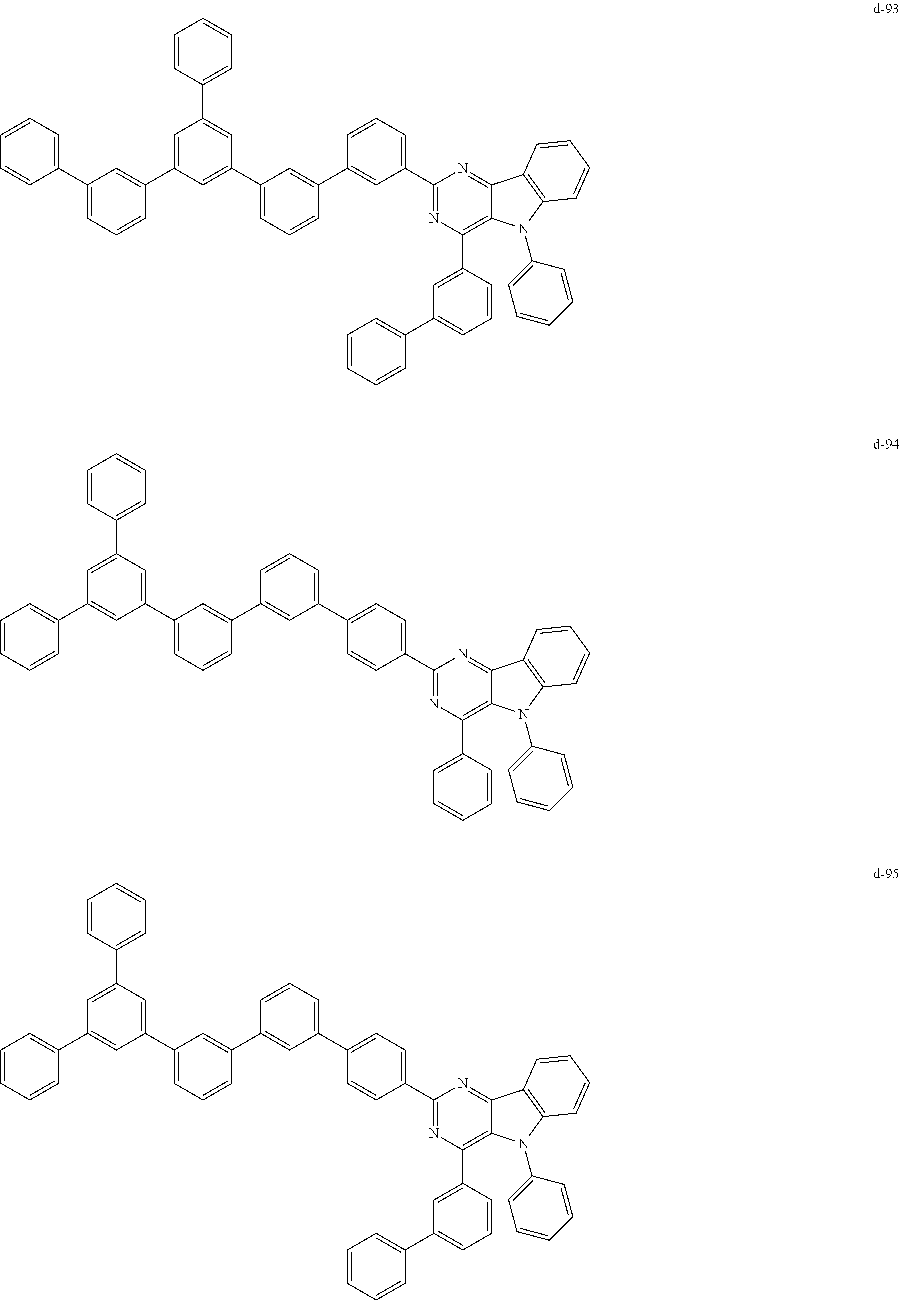 [SMILES: c1ccc(-c2cccc(-c3cc(-c4ccccc4)cc(-c4cccc(-c5cccc(-c6nc(-c7cccc(-c8ccccc8)c7)c7c(n6)c6ccccc6n7-c6ccccc6)c5)c4)c3)c2)cc1 ; c1ccc(-c2cc(-c3ccccc3)cc(-c3cccc(-c4cccc(-c5ccc(-c6nc(-c7ccccc7)c7c(n6)c6ccccc6n7-c6ccccc6)cc5)c4)c3)c2)cc1 ; c1ccc(-c2cc(-c3ccccc3)cc(-c3cccc(-c4cccc(-c5ccc(-c6nc(-c7cccc(-c8ccccc8)c7)c7c(n6)c6ccccc6n7-c6ccccc6)cc5)c4)c3)c2)cc1]

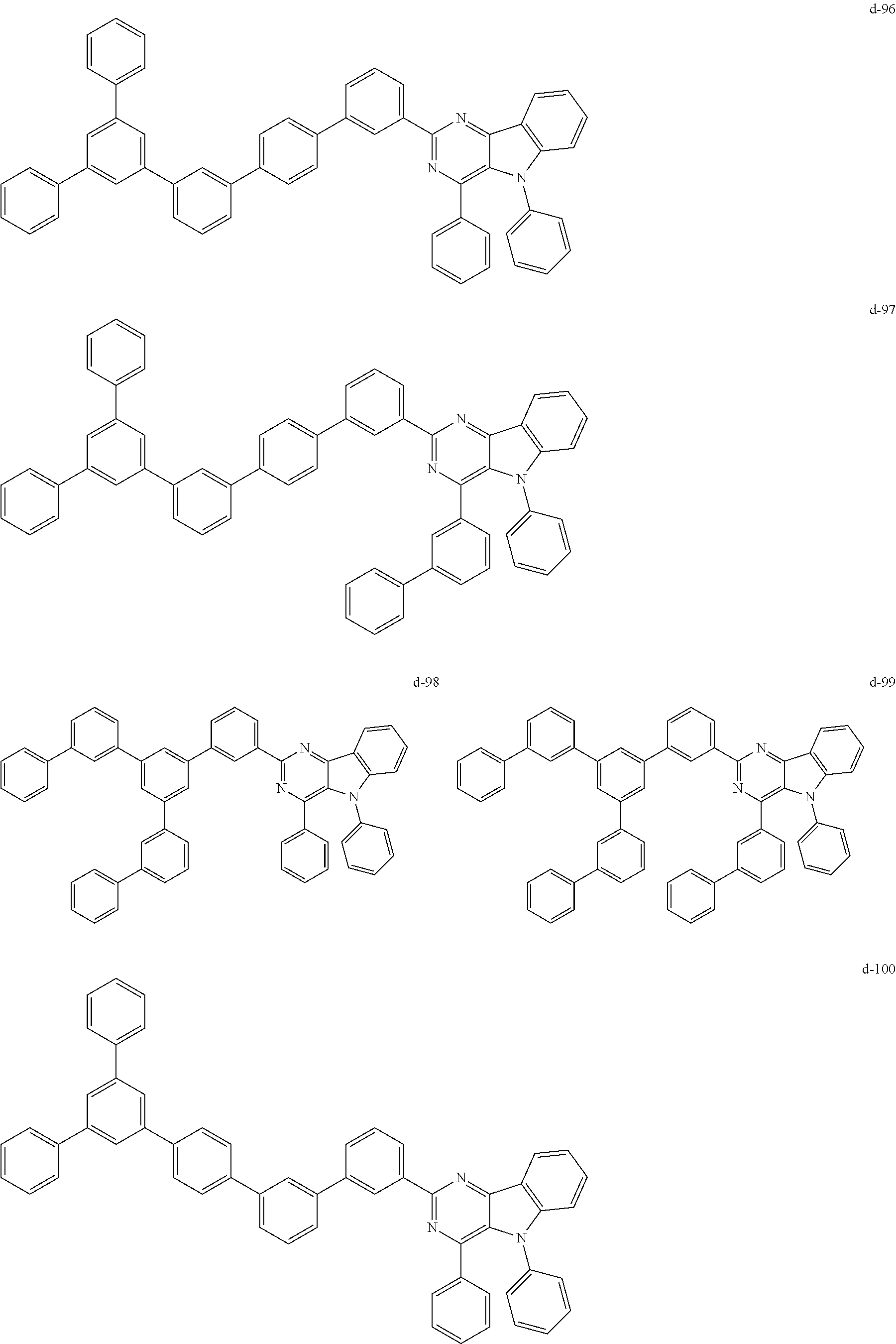

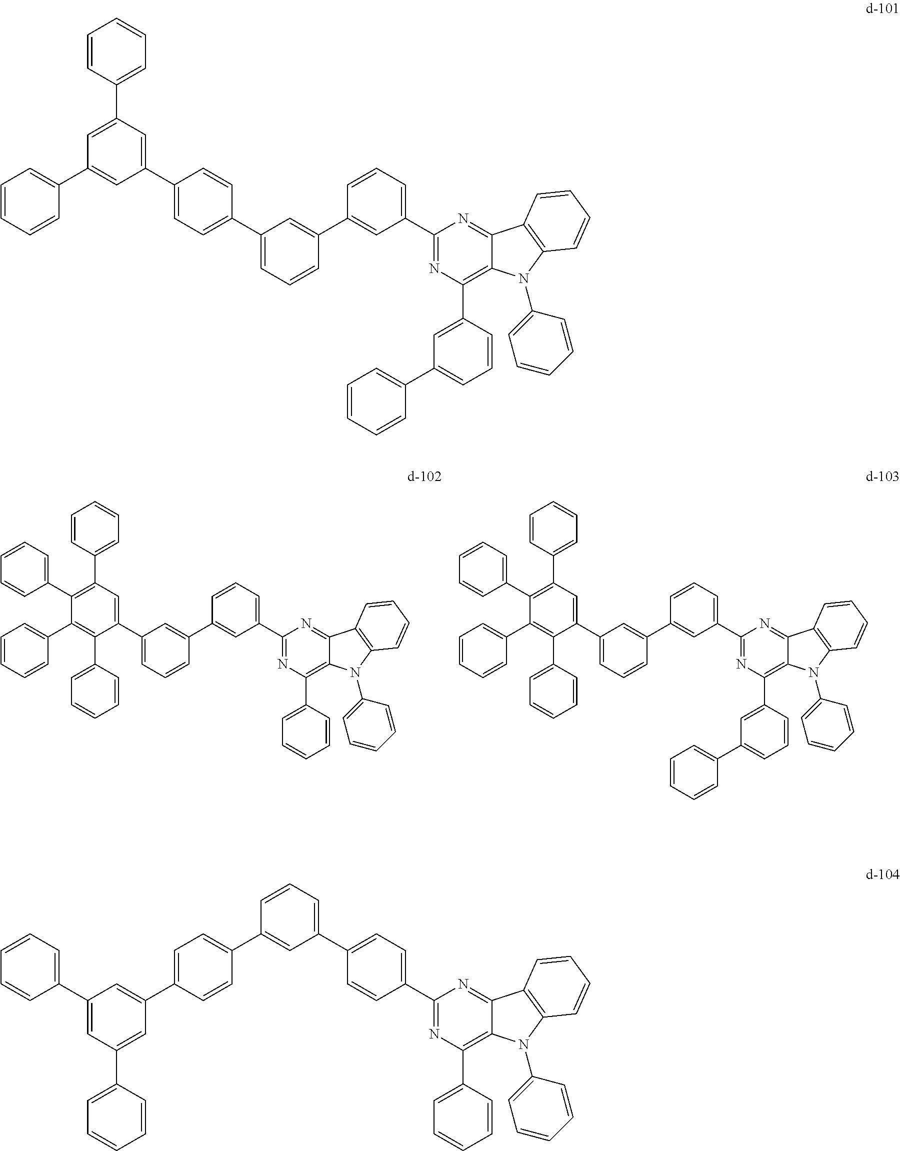 [SMILES: c1ccc(-c2cc(-c3ccccc3)cc(-c3ccc(-c4cccc(-c5cccc(-c6nc(-c7cccc(-c8ccccc8)c7)c7c(n6)c6ccccc6n7-c6ccccc6)c5)c4)cc3)c2)cc1 ; c1ccc(-c2cc(-c3cccc(-c4cccc(-c5nc(-c6ccccc6)c6c(n5)c5ccccc5n6-c5ccccc5)c4)c3)c(-c3ccccc3)c(-c3ccccc3)c2-c2ccccc2)cc1 ; c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4c5ccccc5n(-c5ccccc5)c34)c2)cc1 ; c1ccc(-c2cc(-c3ccccc3)cc(-c3ccc(-c4cccc(-c5ccc(-c6nc(-c7ccccc7)c7c(n6)c6ccccc6n7-c6ccccc6)cc5)c4)cc3)c2)cc1]

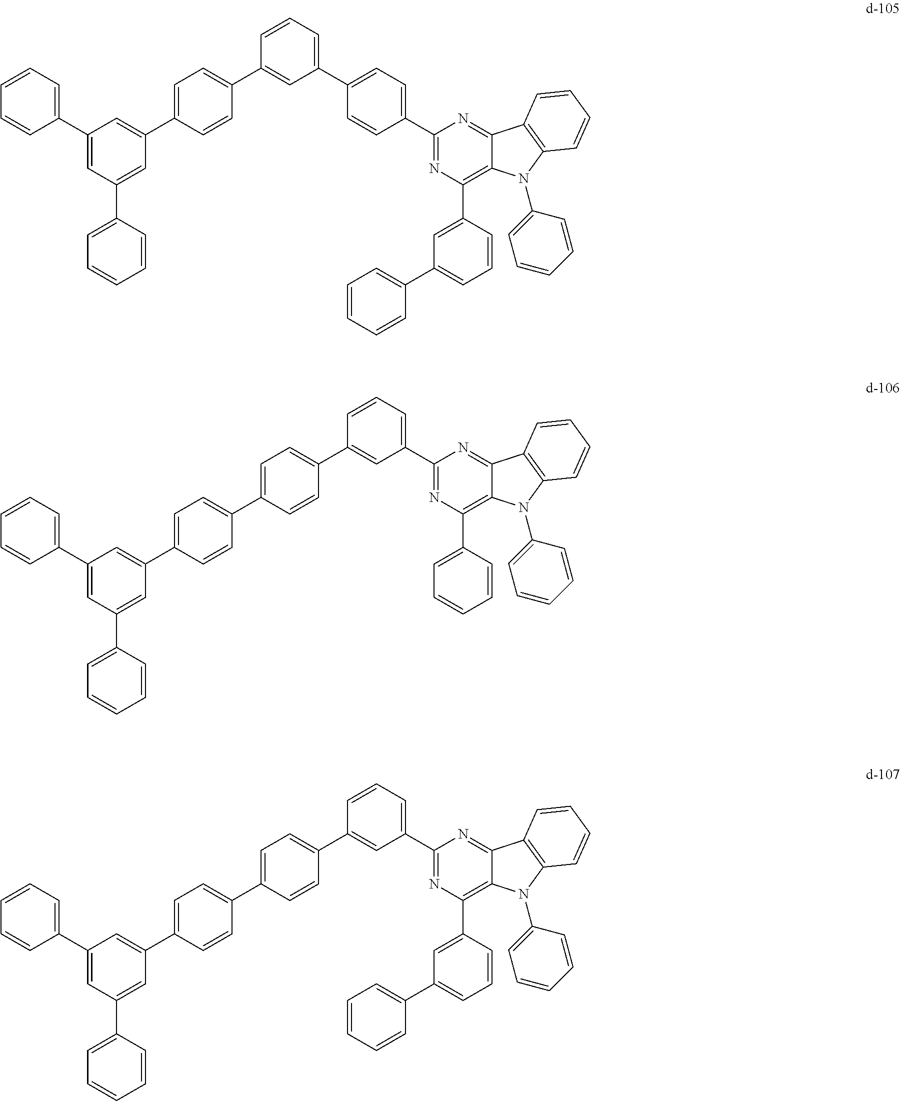 [SMILES: c1ccc(-c2cc(-c3ccccc3)cc(-c3ccc(-c4cccc(-c5ccc(-c6nc(-c7cccc(-c8ccccc8)c7)c7c(n6)c6ccccc6n7-c6ccccc6)cc5)c4)cc3)c2)cc1 ; c1ccc(-c2cc(-c3ccccc3)cc(-c3ccc(-c4ccc(-c5cccc(-c6nc(-c7ccccc7)c7c(n6)c6ccccc6n7-c6ccccc6)c5)cc4)cc3)c2)cc1 ; c1ccc(-c2cc(-c3ccccc3)cc(-c3ccc(-c4ccc(-c5cccc(-c6nc(-c7cccc(-c8ccccc8)c7)c7c(n6)c6ccccc6n7-c6ccccc6)c5)cc4)cc3)c2)cc1]

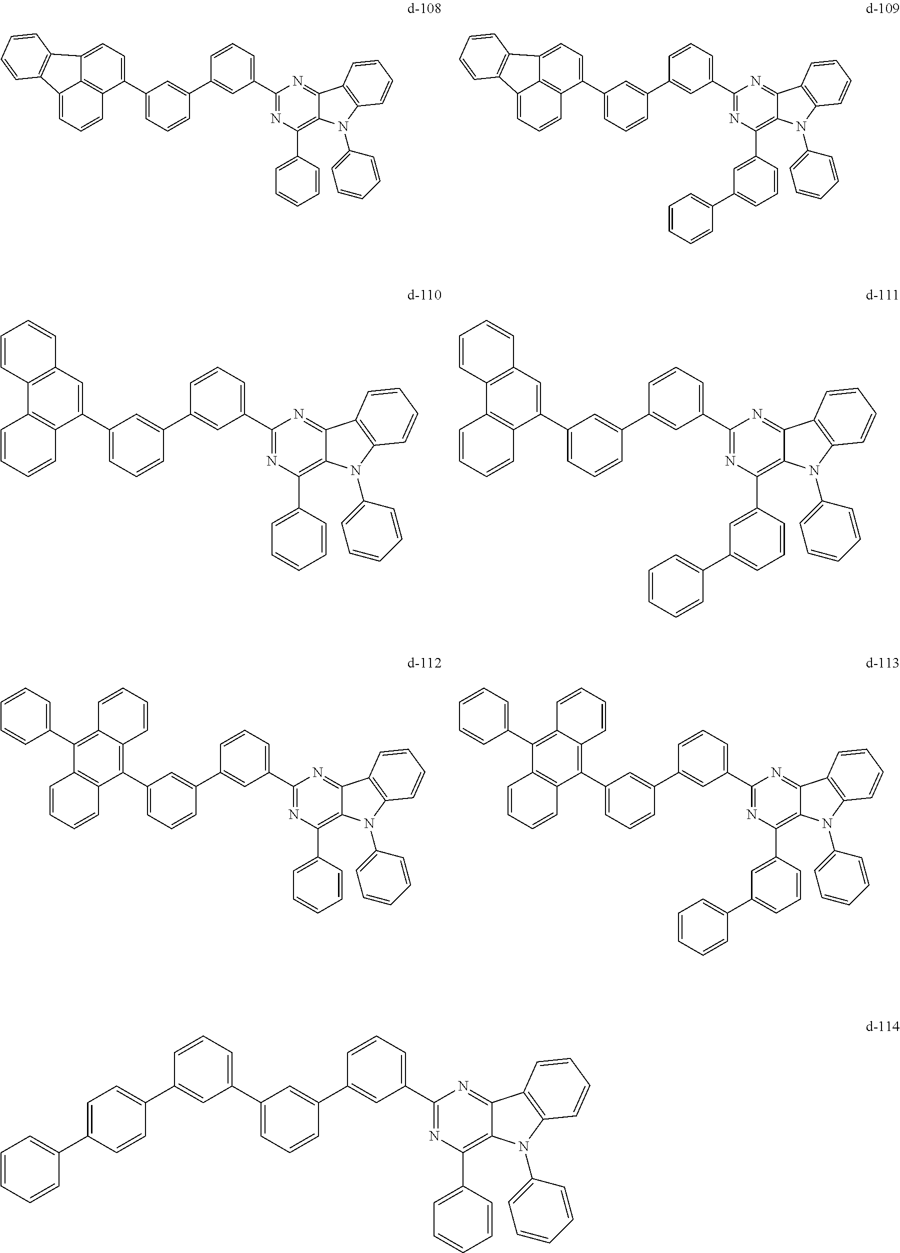

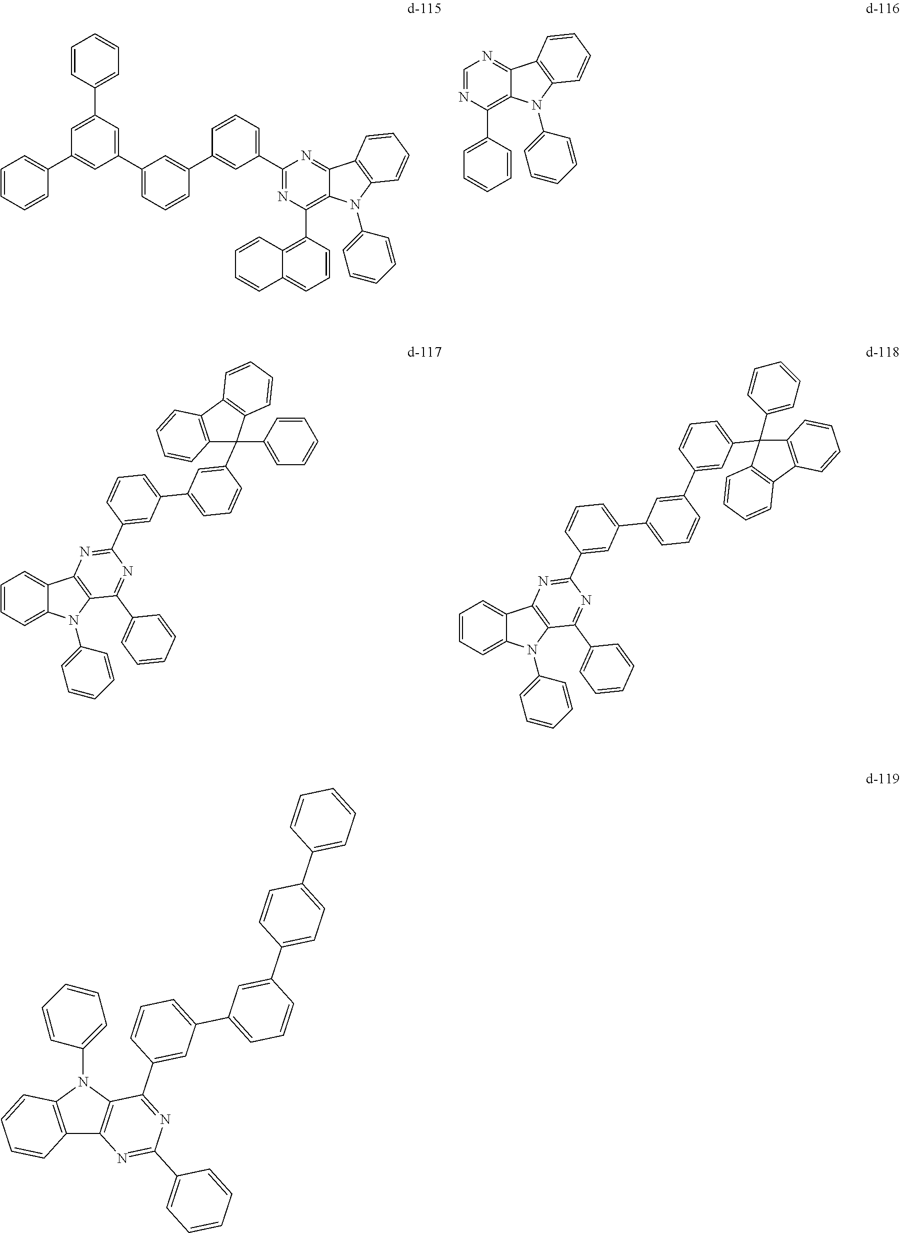 [SMILES: c1ccc(-c2cc(-c3ccccc3)cc(-c3cccc(-c4cccc(-c5nc(-c6cccc7ccccc67)c6c(n5)c5ccccc5n6-c5ccccc5)c4)c3)c2)cc1 ; c1ccc(-c2ncnc3c4ccccc4n(-c4ccccc4)c23)cc1 ; c1ccc(-c2nc(-c3cccc(-c4cccc(C5(c6ccccc6)c6ccccc6-c6ccccc65)c4)c3)nc3c4ccccc4n(-c4ccccc4)c23)cc1 ; c1ccc(-c2nc(-c3cccc(-c4cccc(-c5cccc(C6(c7ccccc7)c7ccccc7-c7ccccc76)c5)c4)c3)nc3c4ccccc4n(-c4ccccc4)c23)cc1 ; c1ccc(-c2ccc(-c3cccc(-c4cccc(-c5nc(-c6ccccc6)nc6c7ccccc7n(-c7ccccc7)c56)c4)c3)cc2)cc1]

Group of X₁=O in Formula 1-2

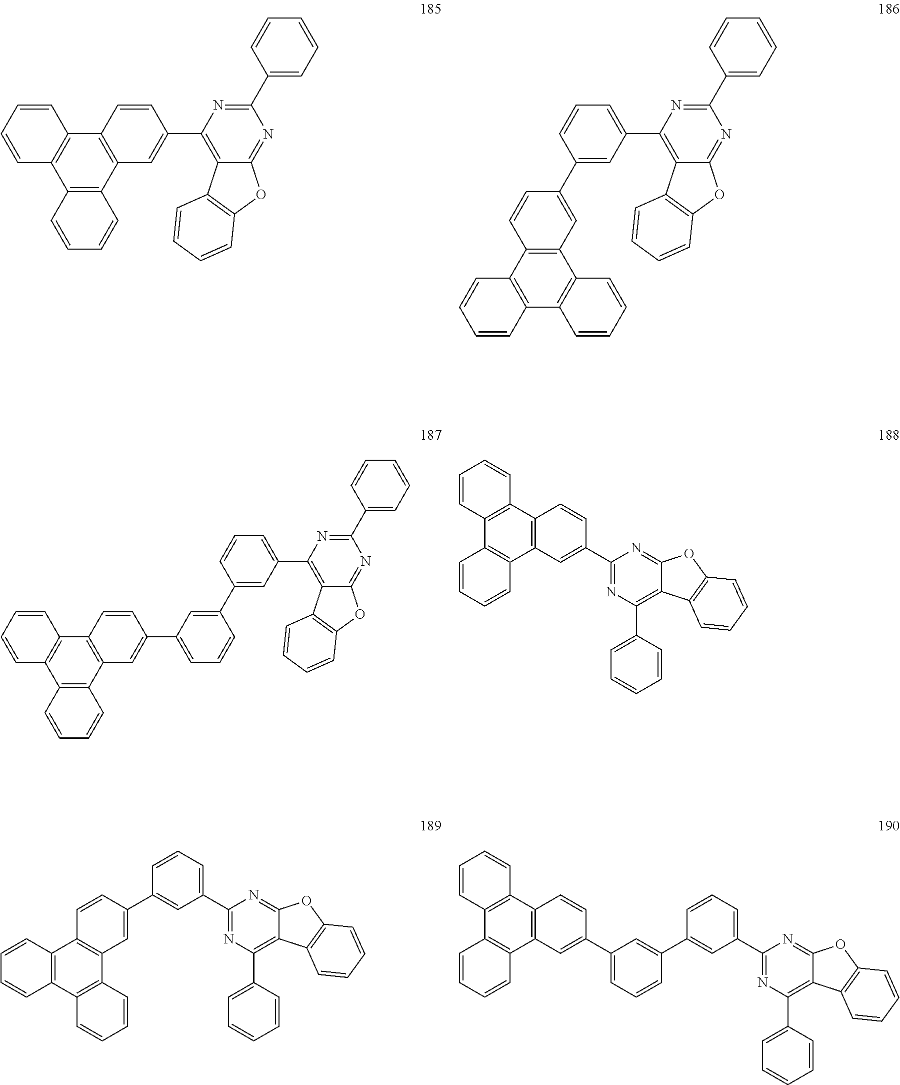 [SMILES: c1ccc(-c2nc(-c3ccc4c5ccccc5c5ccccc5c4c3)c3c(n2)oc2ccccc23)cc1 ; c1ccc(-c2nc(-c3cccc(-c4ccc5c6ccccc6c6ccccc6c5c4)c3)c3c(n2)oc2ccccc23)cc1 ; c1ccc(-c2nc(-c3cccc(-c4cccc(-c5ccc6c7ccccc7c7ccccc7c6c5)c4)c3)c3c(n2)oc2ccccc23)cc1 ; c1ccc(-c2nc(-c3ccc4c5ccccc5c5ccccc5c4c3)nc3oc4ccccc4c23)cc1 ; c1ccc(-c2nc(-c3cccc(-c4ccc5c6ccccc6c6ccccc6c5c4)c3)nc3oc4ccccc4c23)cc1 ; c1ccc(-c2nc(-c3cccc(-c4cccc(-c5ccc6c7ccccc7c7ccccc7c6c5)c4)c3)nc3oc4ccccc4c23)cc1]

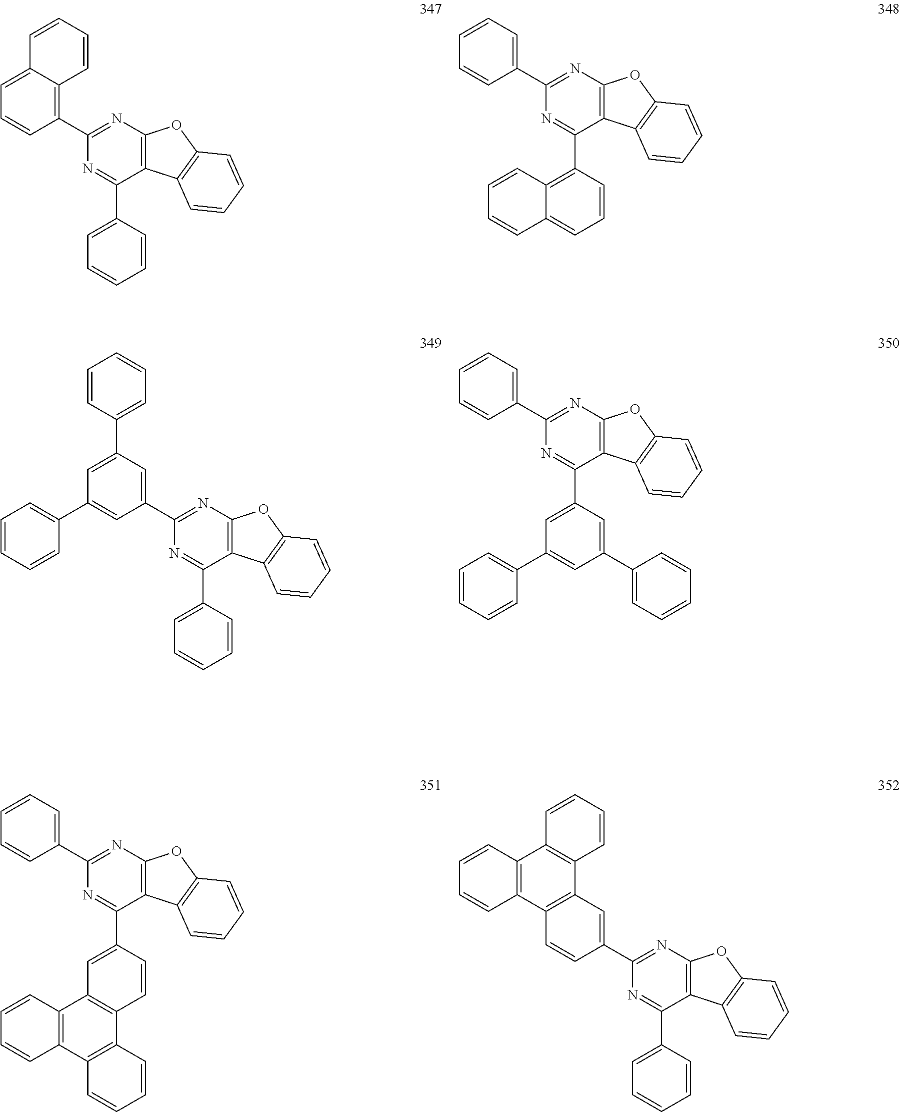 [SMILES: c1ccc(-c2nc(-c3cccc4ccccc34)nc3oc4ccccc4c23)cc1 ; c1ccc(-c2nc(-c3cccc4ccccc34)c3c(n2)oc2ccccc23)cc1 ; c1ccc(-c2cc(-c3ccccc3)cc(-c3nc(-c4ccccc4)c4c(n3)oc3ccccc34)c2)cc1 ; c1ccc(-c2cc(-c3ccccc3)cc(-c3nc(-c4ccccc4)nc4oc5ccccc5c34)c2)cc1 ; c1ccc(-c2nc(-c3ccc4c5ccccc5c5ccccc5c4c3)c3c(n2)oc2ccccc23)cc1 ; c1ccc(-c2nc(-c3ccc4c5ccccc5c5ccccc5c4c3)nc3oc4ccccc4c23)cc1]

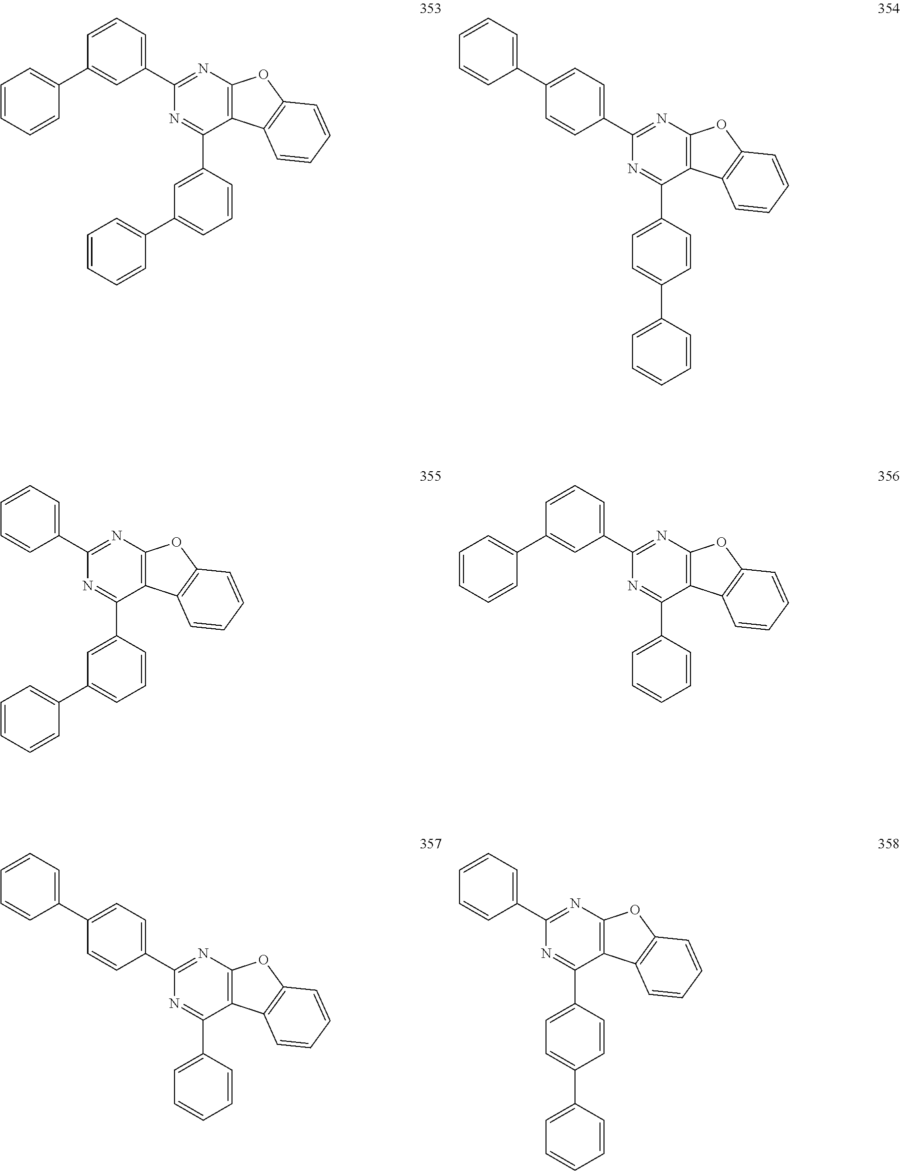 [SMILES: c1ccc(-c2cccc(-c3nc(-c4cccc(-c5ccccc5)c4)c4c(n3)oc3ccccc34)c2)cc1 ; c1ccc(-c2ccc(-c3nc(-c4ccc(-c5ccccc5)cc4)c4c(n3)oc3ccccc34)cc2)cc1 ; c1ccc(-c2cccc(-c3nc(-c4ccccc4)nc4oc5ccccc5c34)c2)cc1 ; c1ccc(-c2cccc(-c3nc(-c4ccccc4)c4c(n3)oc3ccccc34)c2)cc1 ; c1ccc(-c2ccc(-c3nc(-c4ccccc4)c4c(n3)oc3ccccc34)cc2)cc1 ; c1ccc(-c2ccc(-c3nc(-c4ccccc4)nc4oc5ccccc5c34)cc2)cc1]

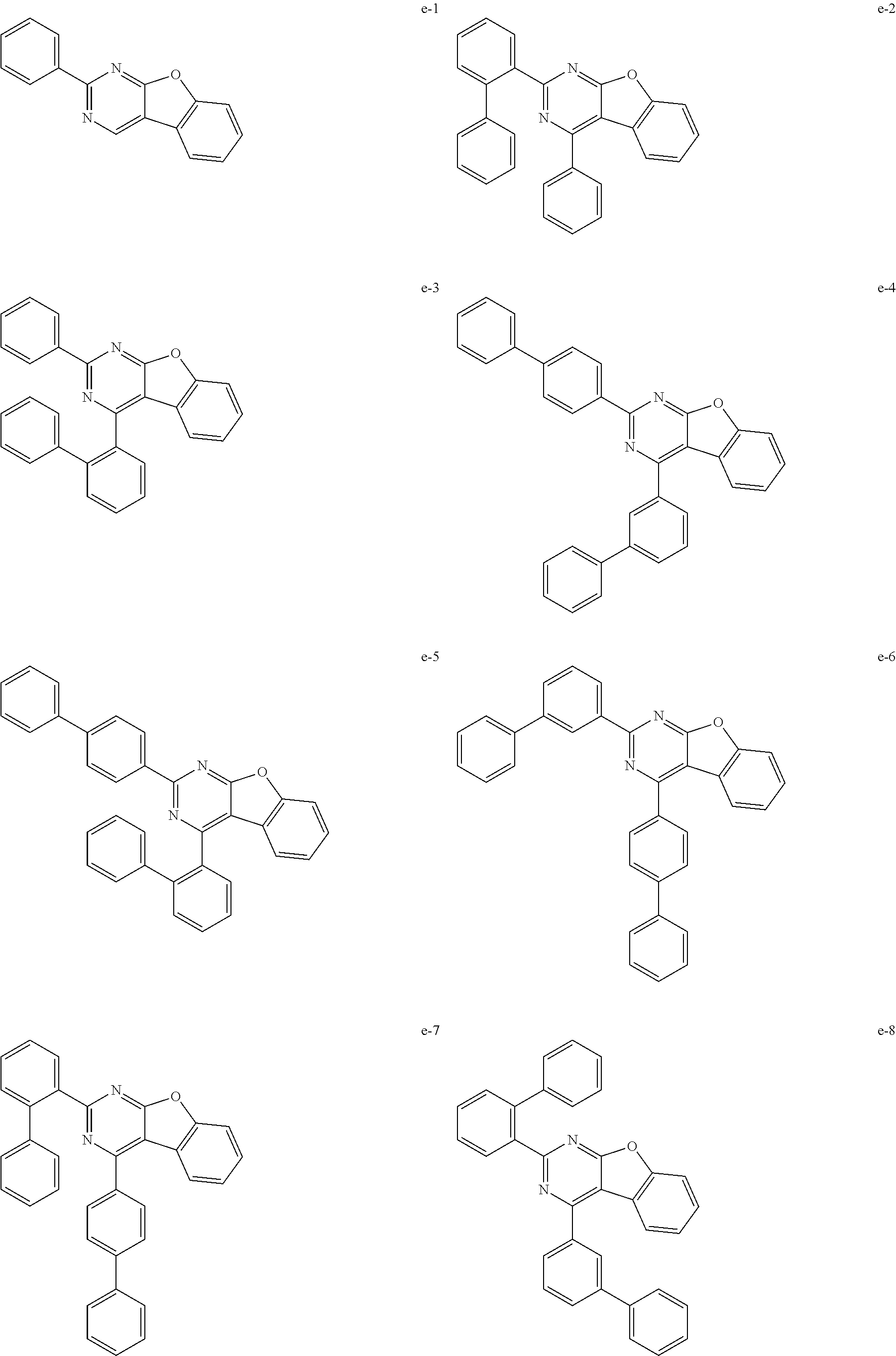

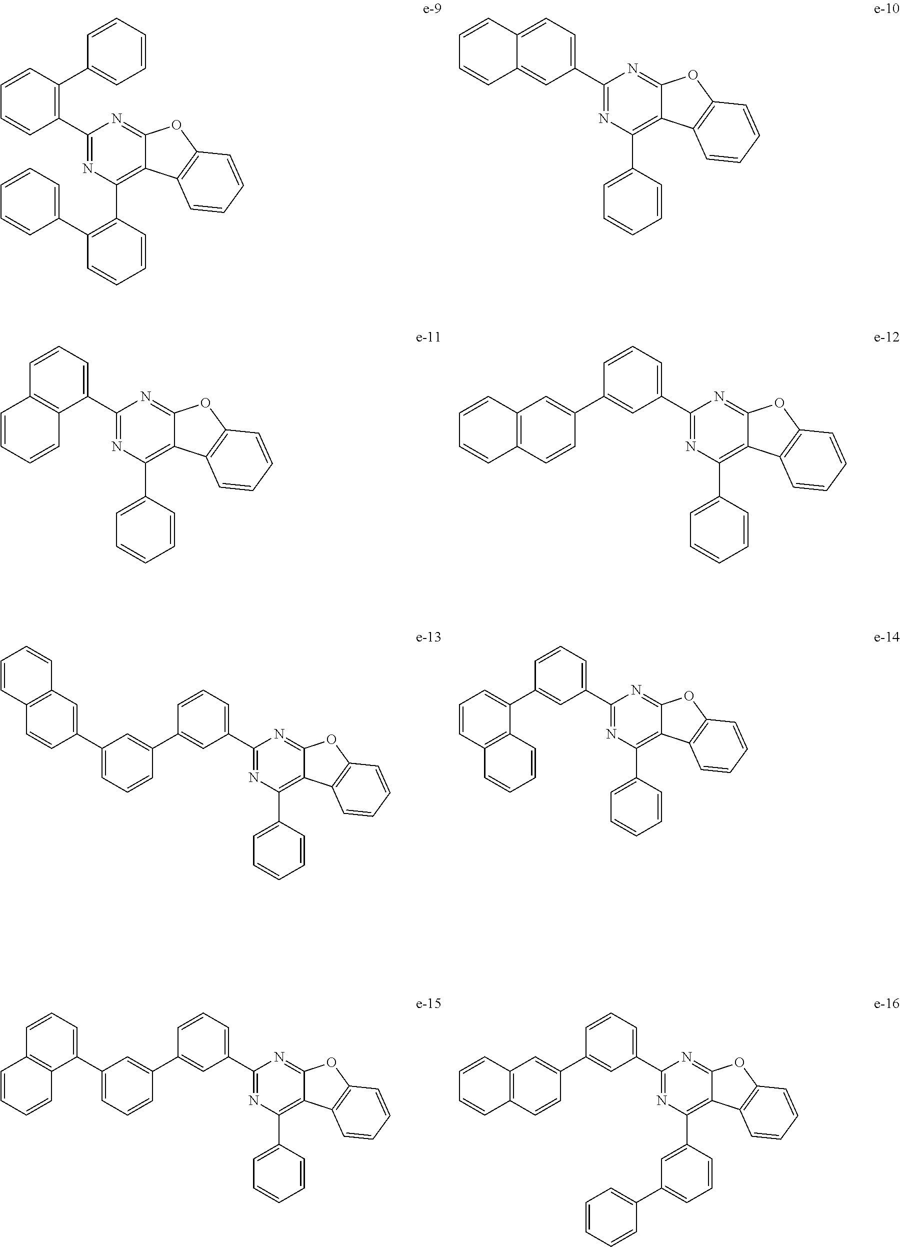

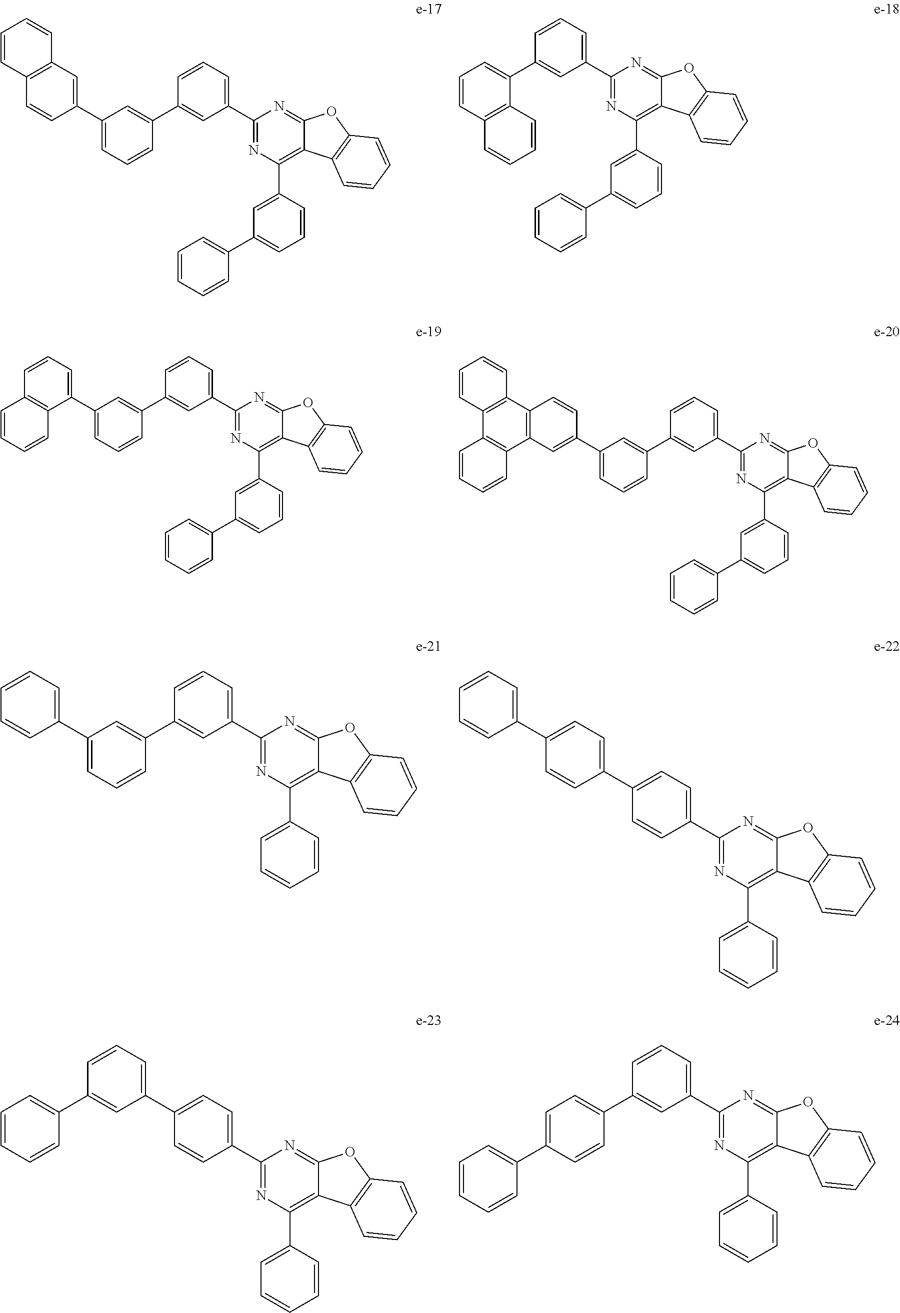

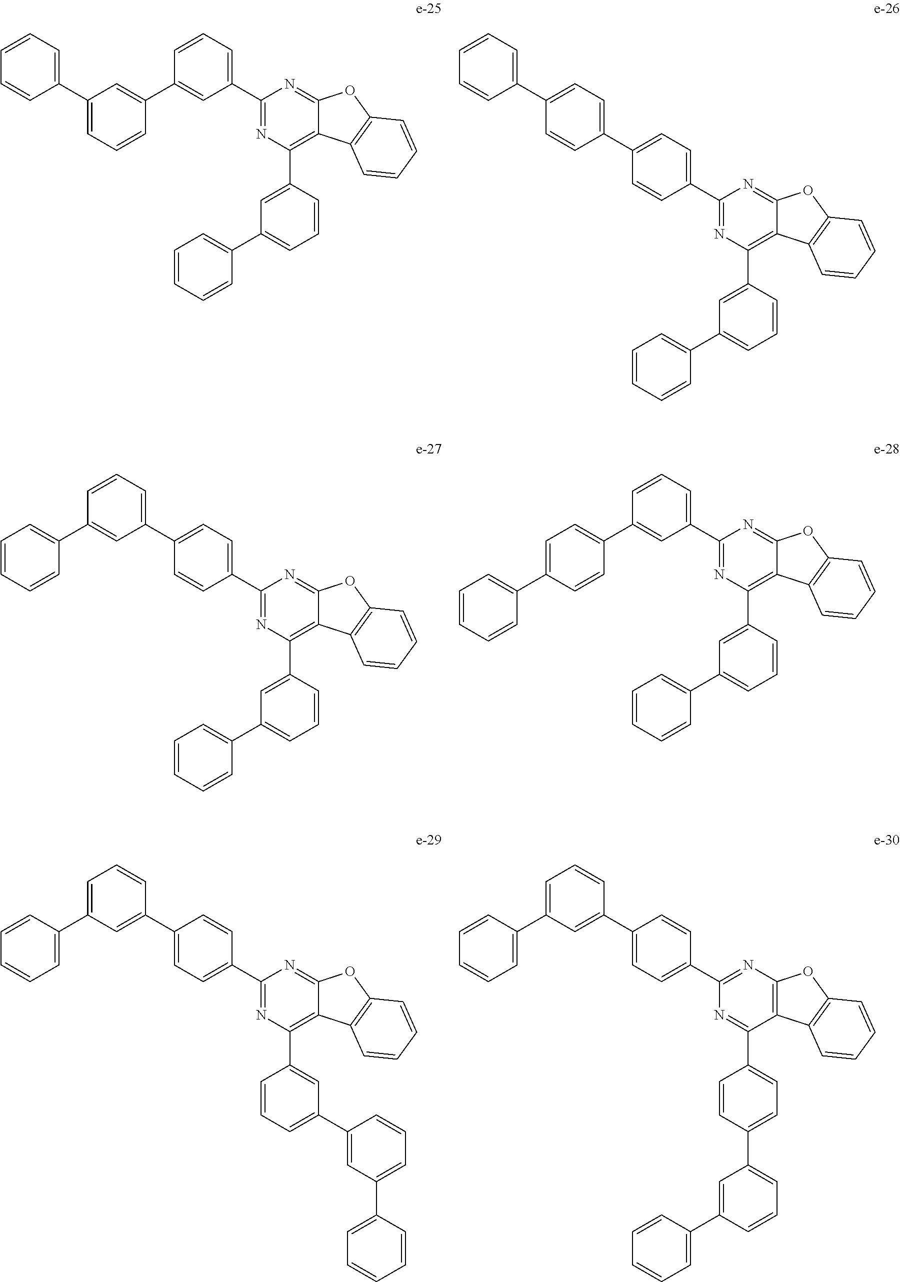

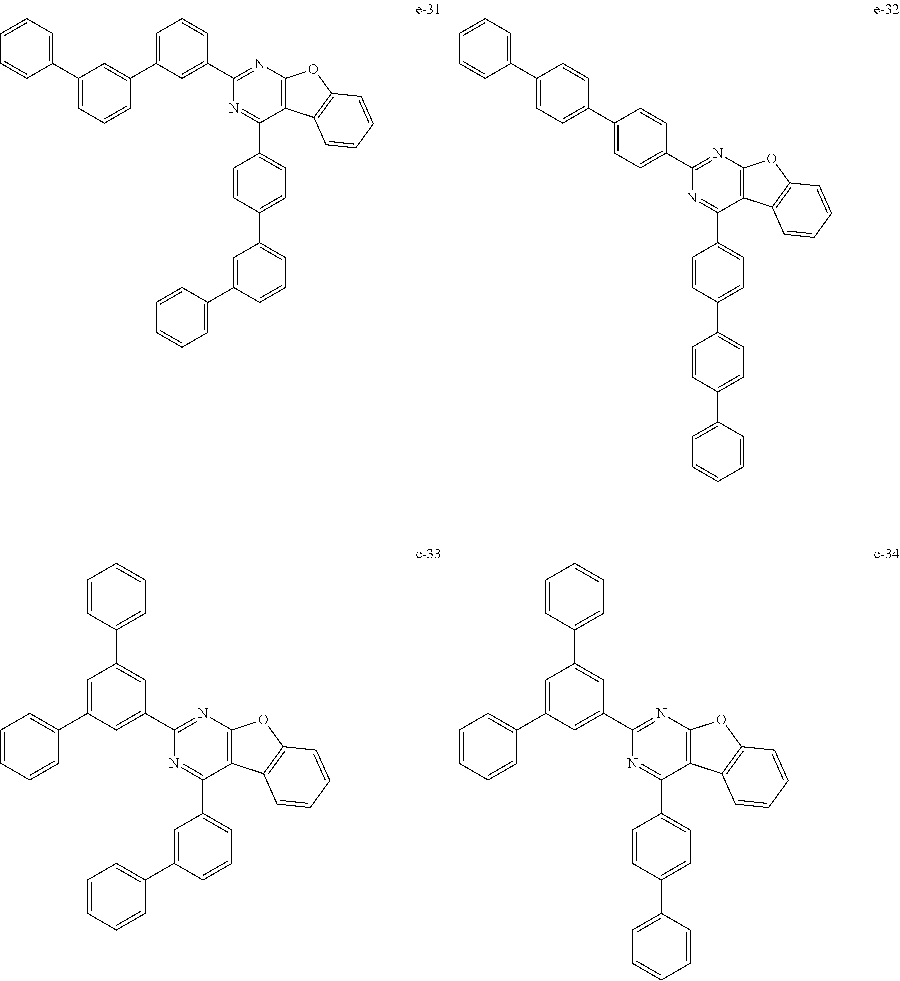 [SMILES: c1ccc(-c2cccc(-c3ccc(-c4nc(-c5cccc(-c6cccc(-c7ccccc7)c6)c5)nc5oc6ccccc6c45)cc3)c2)cc1 ; c1ccc(-c2ccc(-c3ccc(-c4nc(-c5ccc(-c6ccc(-c7ccccc7)cc6)cc5)c5c(n4)oc4ccccc45)cc3)cc2)cc1 ; c1ccc(-c2cc(-c3ccccc3)cc(-c3nc(-c4cccc(-c5ccccc5)c4)c4c(n3)oc3ccccc34)c2)cc1 ; c1ccc(-c2ccc(-c3nc(-c4cc(-c5ccccc5)cc(-c5ccccc5)c4)nc4oc5ccccc5c34)cc2)cc1]

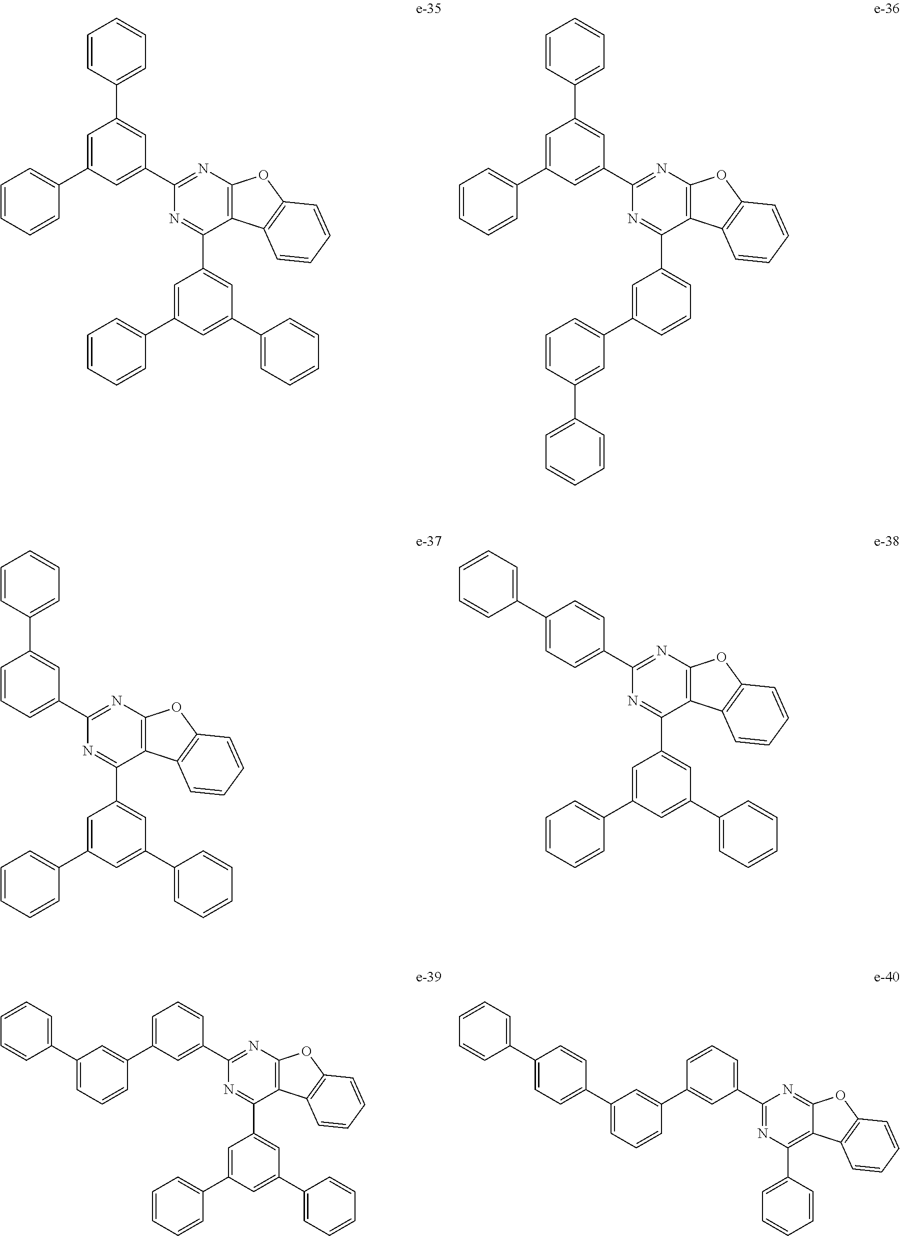

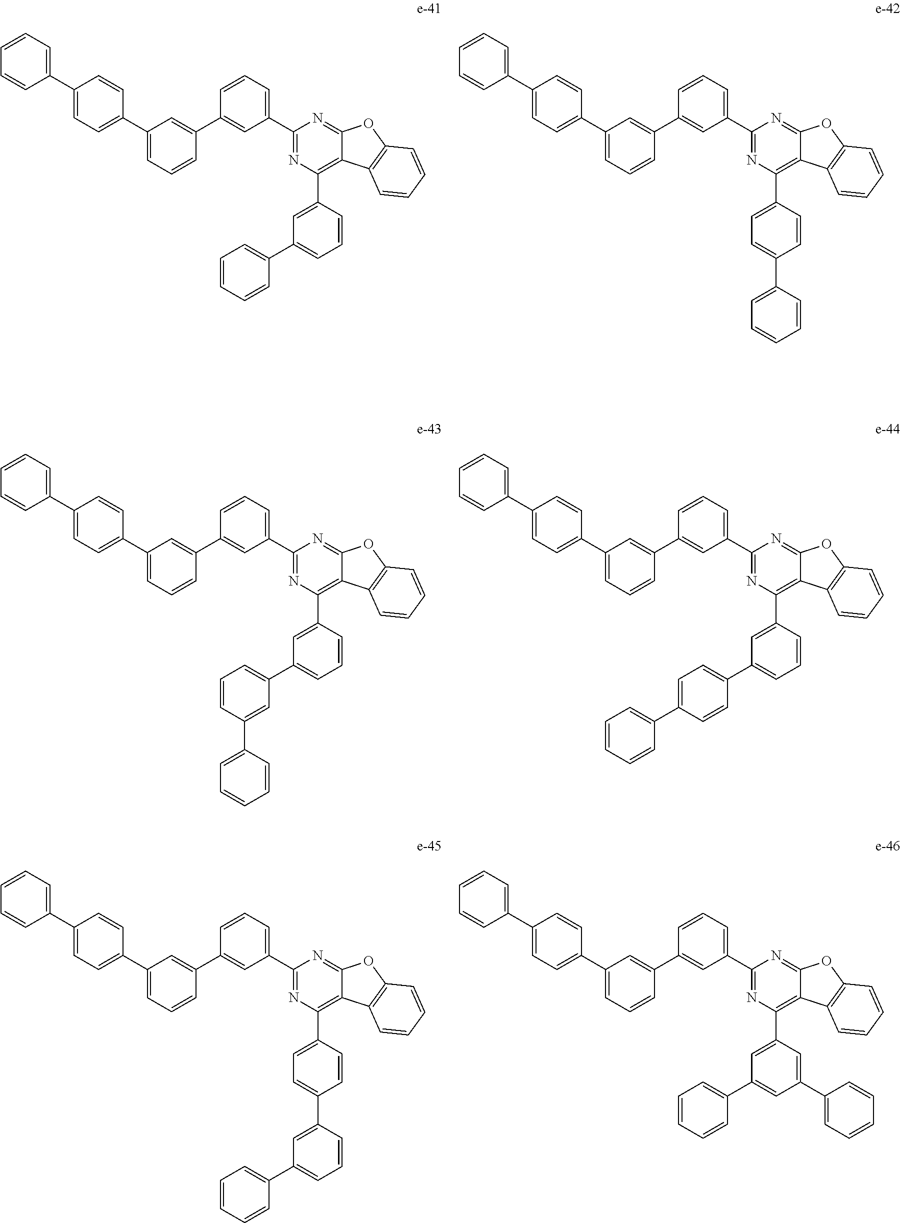

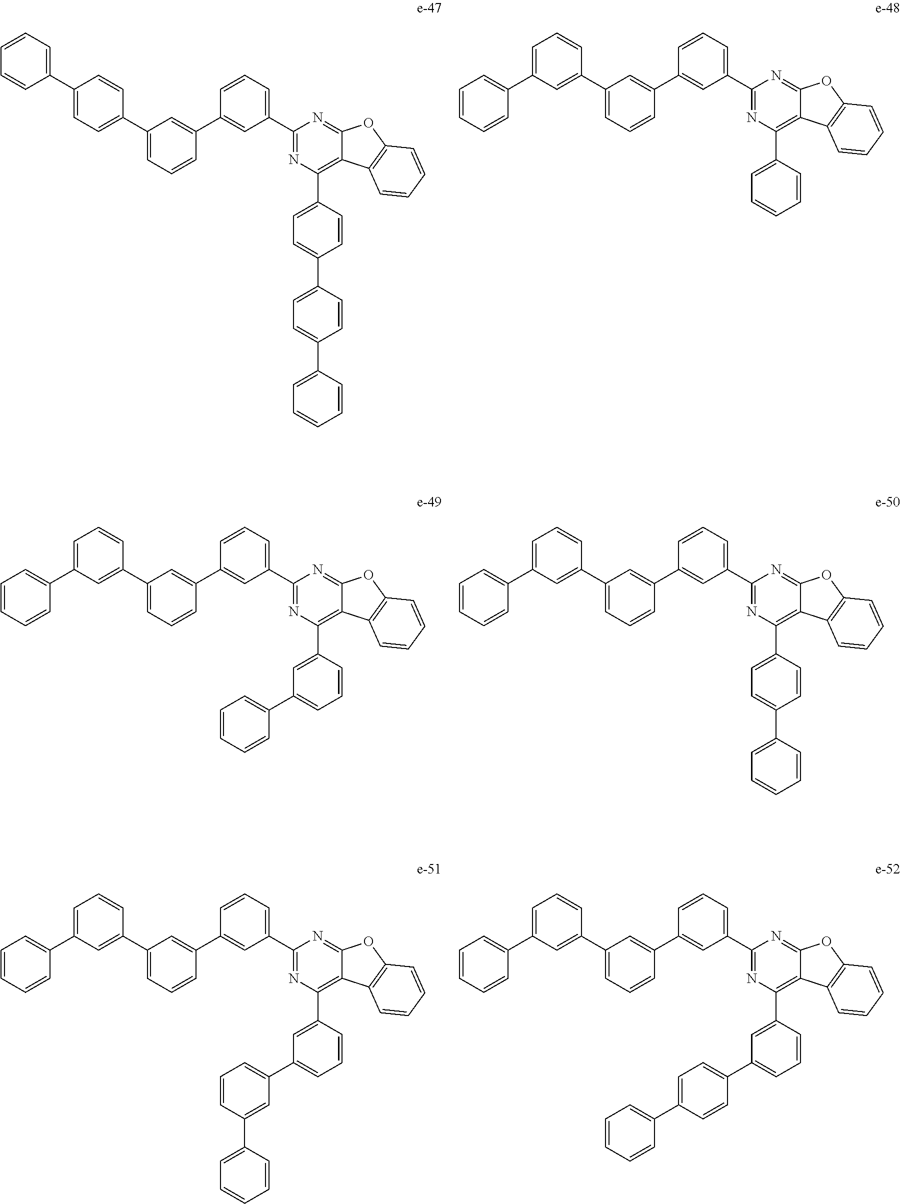

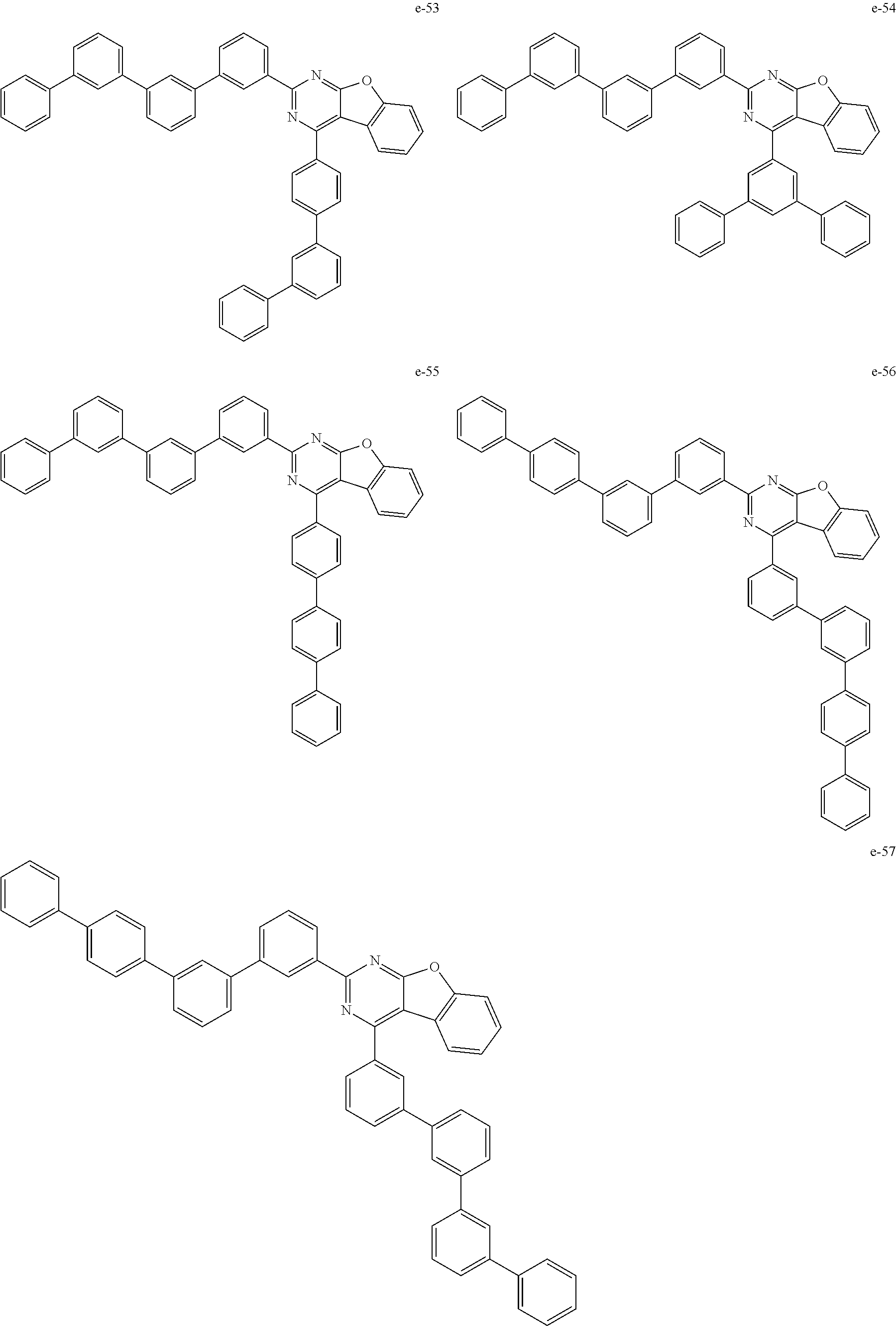

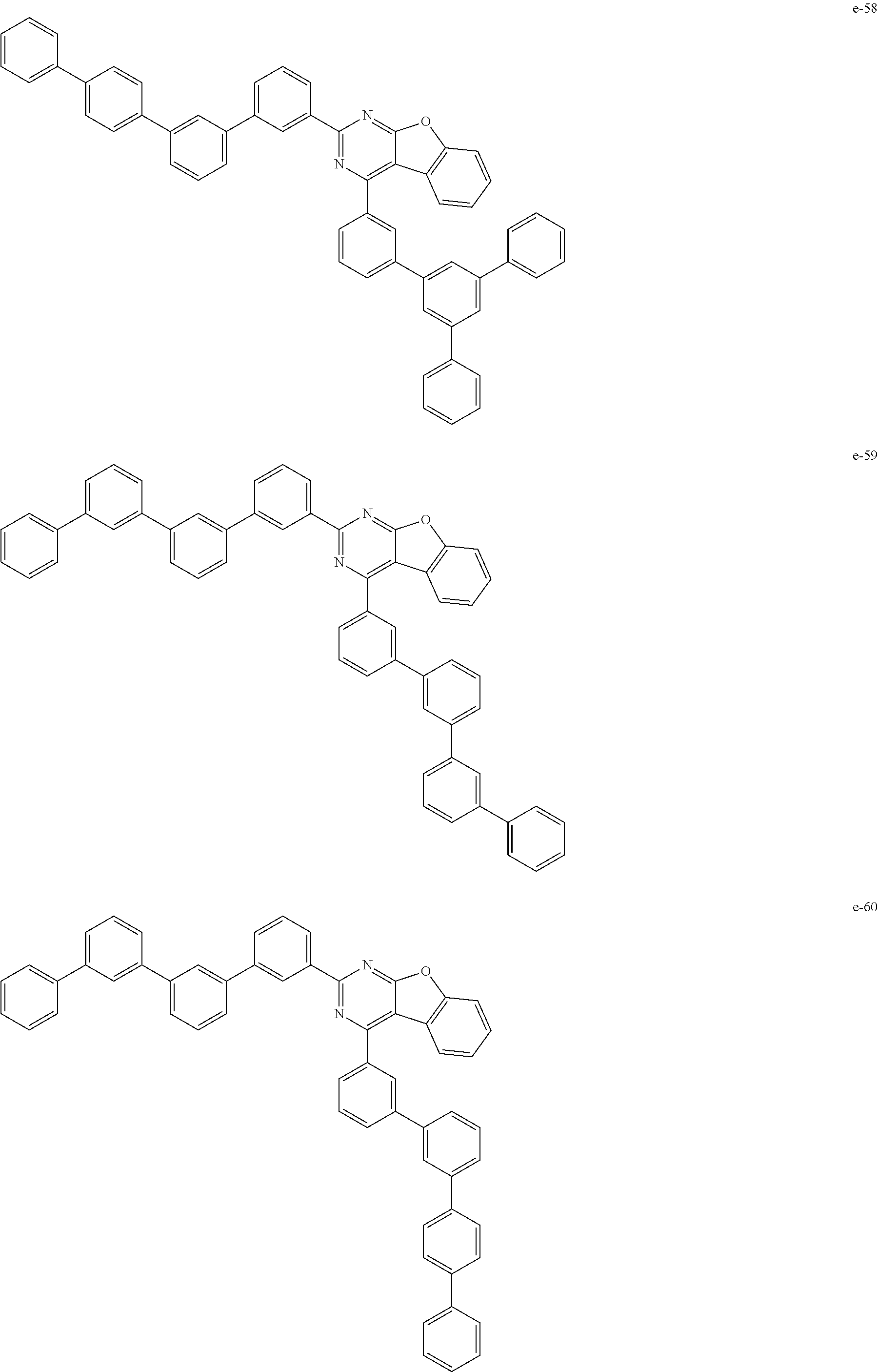 [SMILES: c1ccc(-c2ccc(-c3cccc(-c4cccc(-c5nc(-c6cccc(-c7cc(-c8ccccc8)cc(-c8ccccc8)c7)c6)c6c(n5)oc5ccccc56)c4)c3)cc2)cc1 ; c1ccc(-c2cccc(-c3cccc(-c4cccc(-c5nc(-c6cccc(-c7cccc(-c8cccc(-c9ccccc9)c8)c7)c6)c6c(n5)oc5ccccc56)c4)c3)c2)cc1 ; c1ccc(-c2ccc(-c3cccc(-c4cccc(-c5nc(-c6cccc(-c7cccc(-c8cccc(-c9ccccc9)c8)c7)c6)nc6oc7ccccc7c56)c4)c3)cc2)cc1]

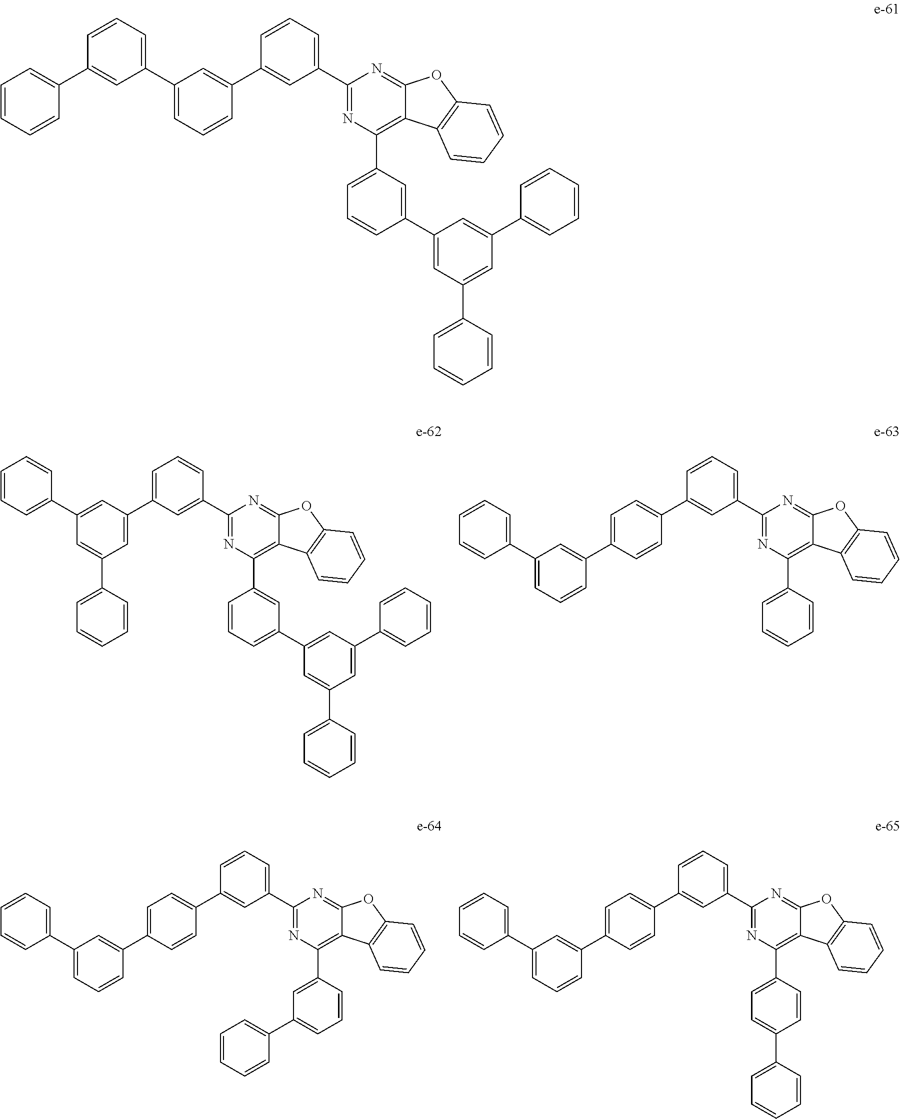

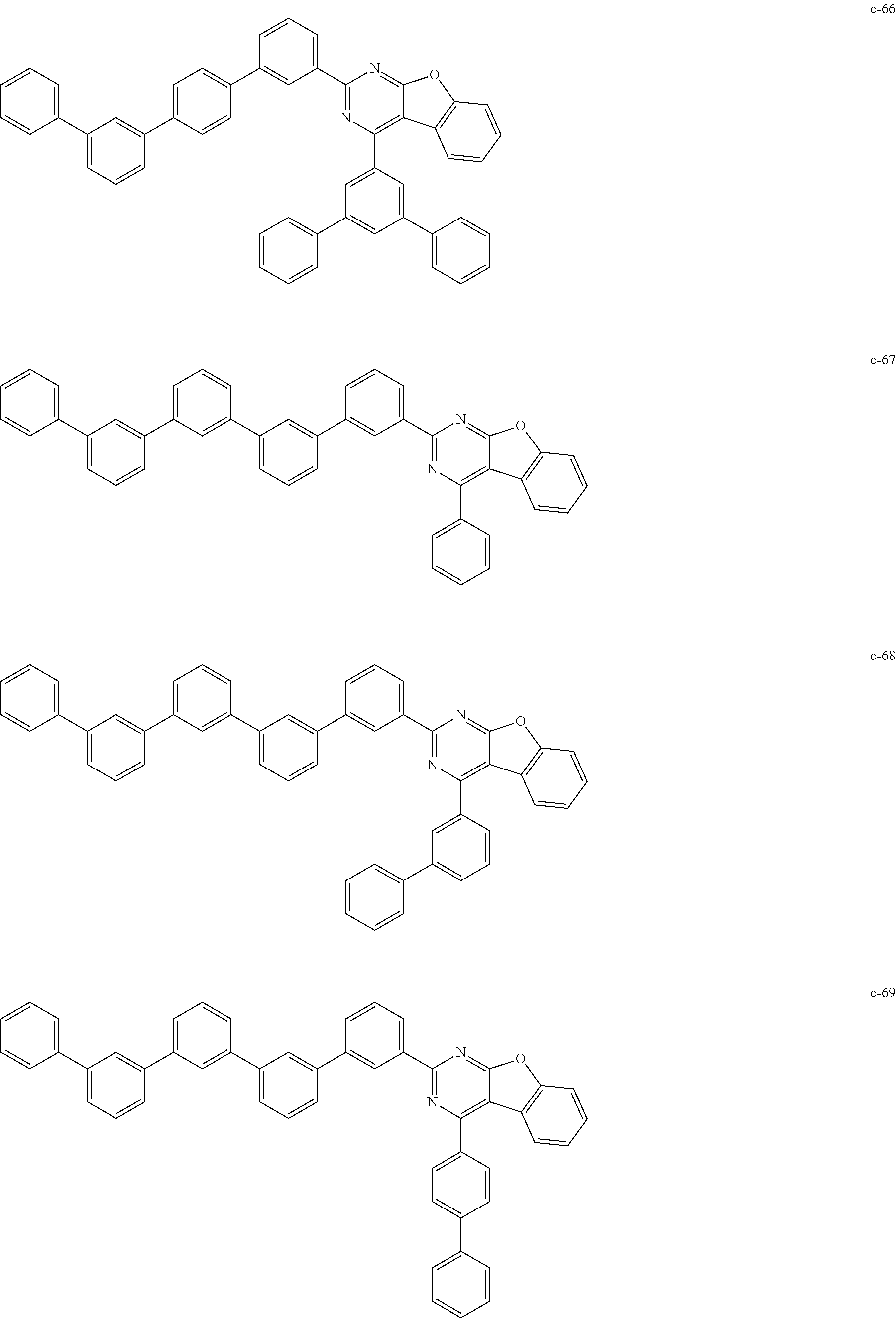 [SMILES: c1ccc(-c2cccc(-c3ccc(-c4cccc(-c5nc(-c6cc(-c7ccccc7)cc(-c7ccccc7)c6)c6c(n5)oc5ccccc56)c4)cc3)c2)cc1 ; c1ccc(-c2cccc(-c3cccc(-c4cccc(-c5cccc(-c6nc(-c7ccccc7)c7c(n6)oc6ccccc67)c5)c4)c3)c2)cc1 ; c1ccc(-c2cccc(-c3cccc(-c4cccc(-c5cccc(-c6nc(-c7cccc(-c8ccccc8)c7)c7c(n6)oc6ccccc67)c5)c4)c3)c2)cc1 ; c1ccc(-c2ccc(-c3nc(-c4cccc(-c5cccc(-c6cccc(-c7cccc(-c8ccccc8)c7)c6)c5)c4)nc4oc5ccccc5c34)cc2)cc1]

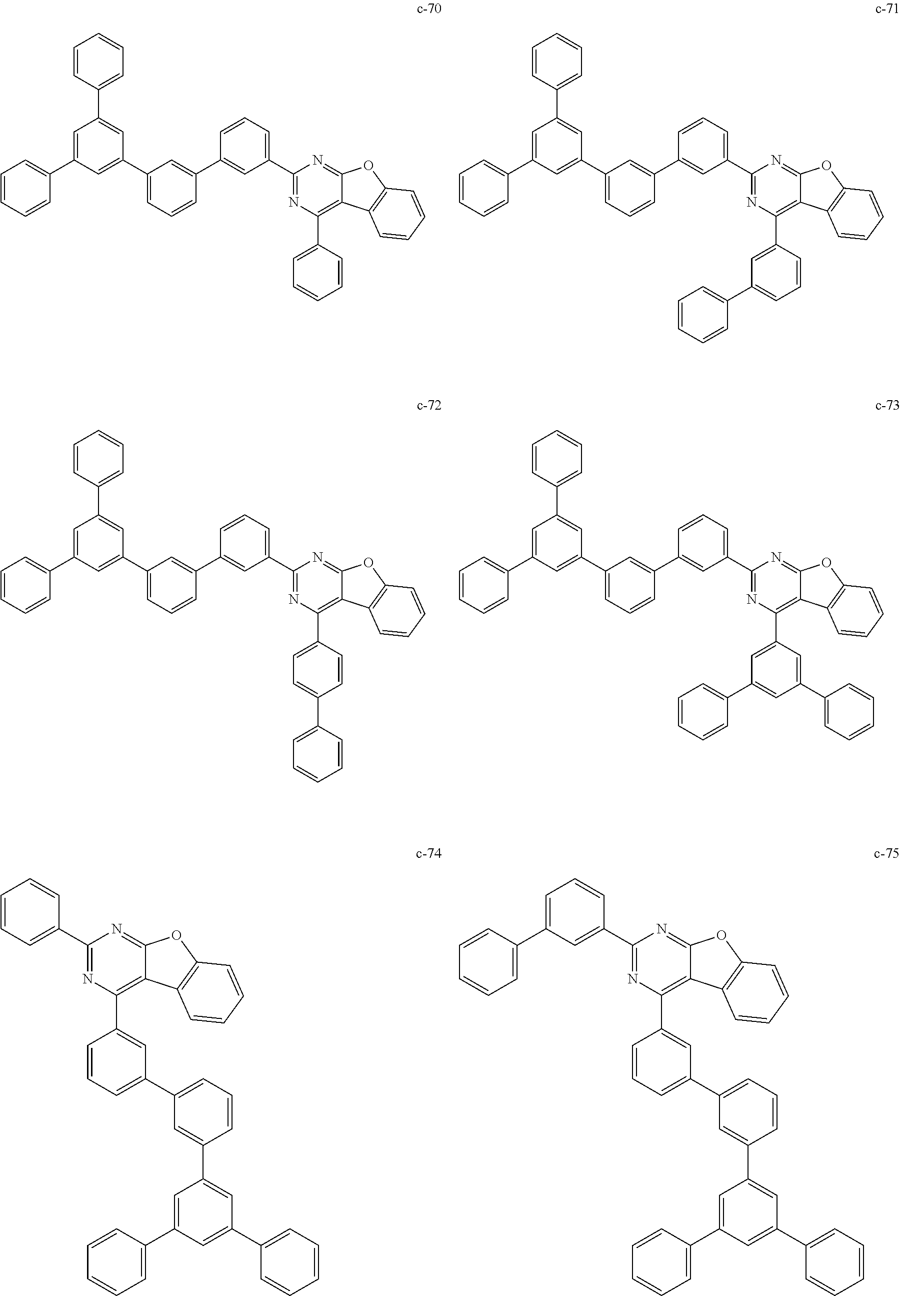

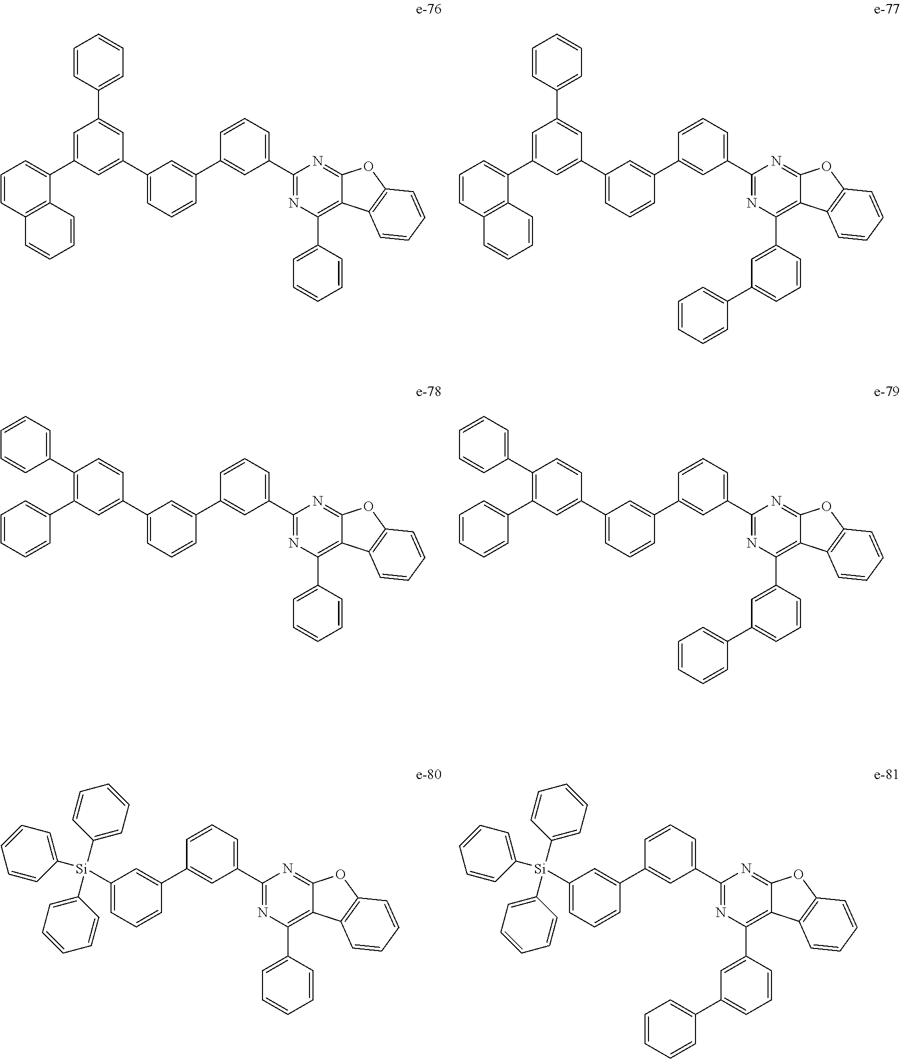

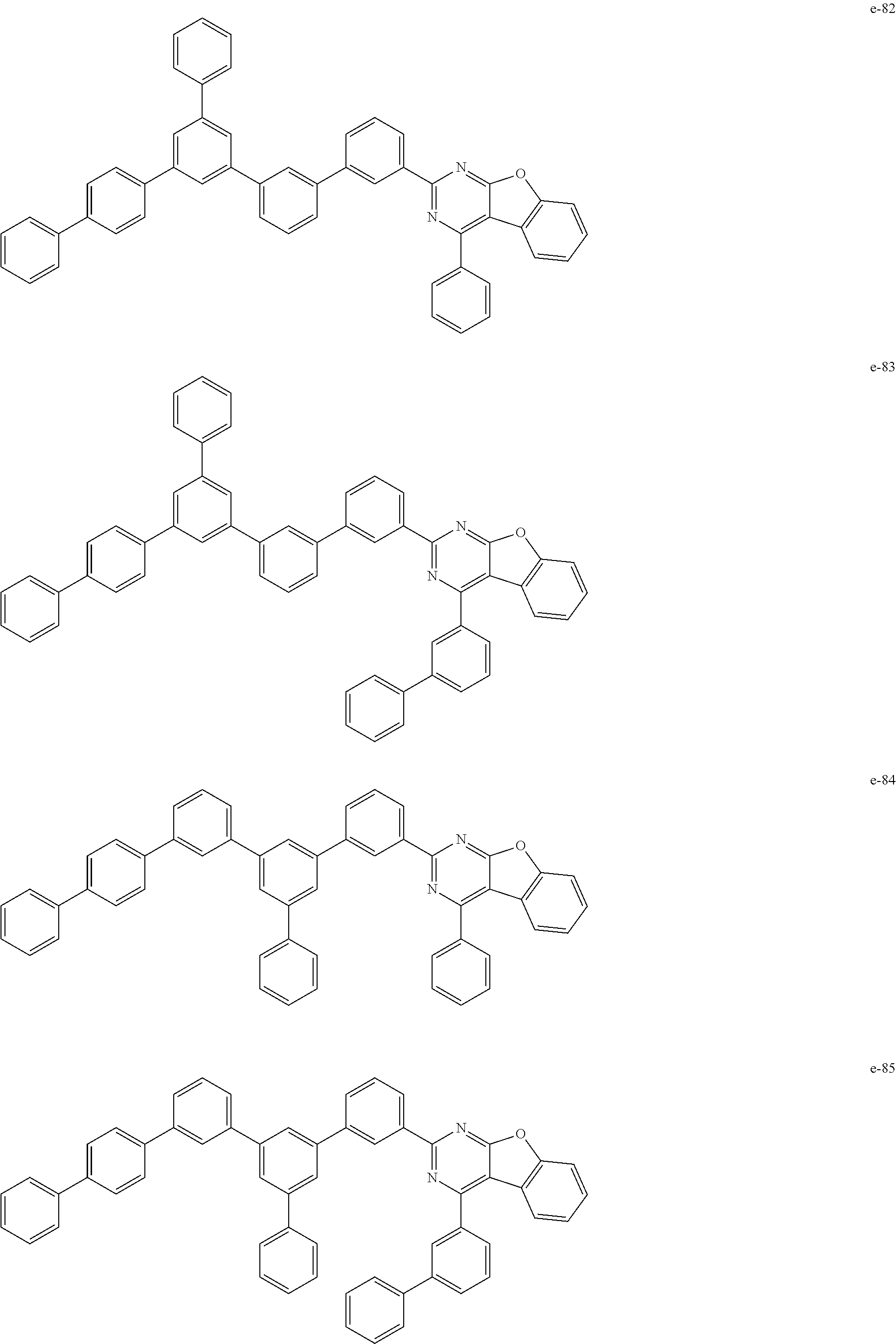 [SMILES: c1ccc(-c2ccc(-c3cc(-c4ccccc4)cc(-c4cccc(-c5cccc(-c6nc(-c7ccccc7)c7c(n6)oc6ccccc67)c5)c4)c3)cc2)cc1 ; c1ccc(-c2ccc(-c3cc(-c4ccccc4)cc(-c4cccc(-c5cccc(-c6nc(-c7cccc(-c8ccccc8)c7)c7c(n6)oc6ccccc67)c5)c4)c3)cc2)cc1 ; c1ccc(-c2ccc(-c3cccc(-c4cc(-c5ccccc5)cc(-c5cccc(-c6nc(-c7ccccc7)c7c(n6)oc6ccccc67)c5)c4)c3)cc2)cc1 ; c1ccc(-c2ccc(-c3cccc(-c4cc(-c5ccccc5)cc(-c5cccc(-c6nc(-c7cccc(-c8ccccc8)c7)c7c(n6)oc6ccccc67)c5)c4)c3)cc2)cc1]

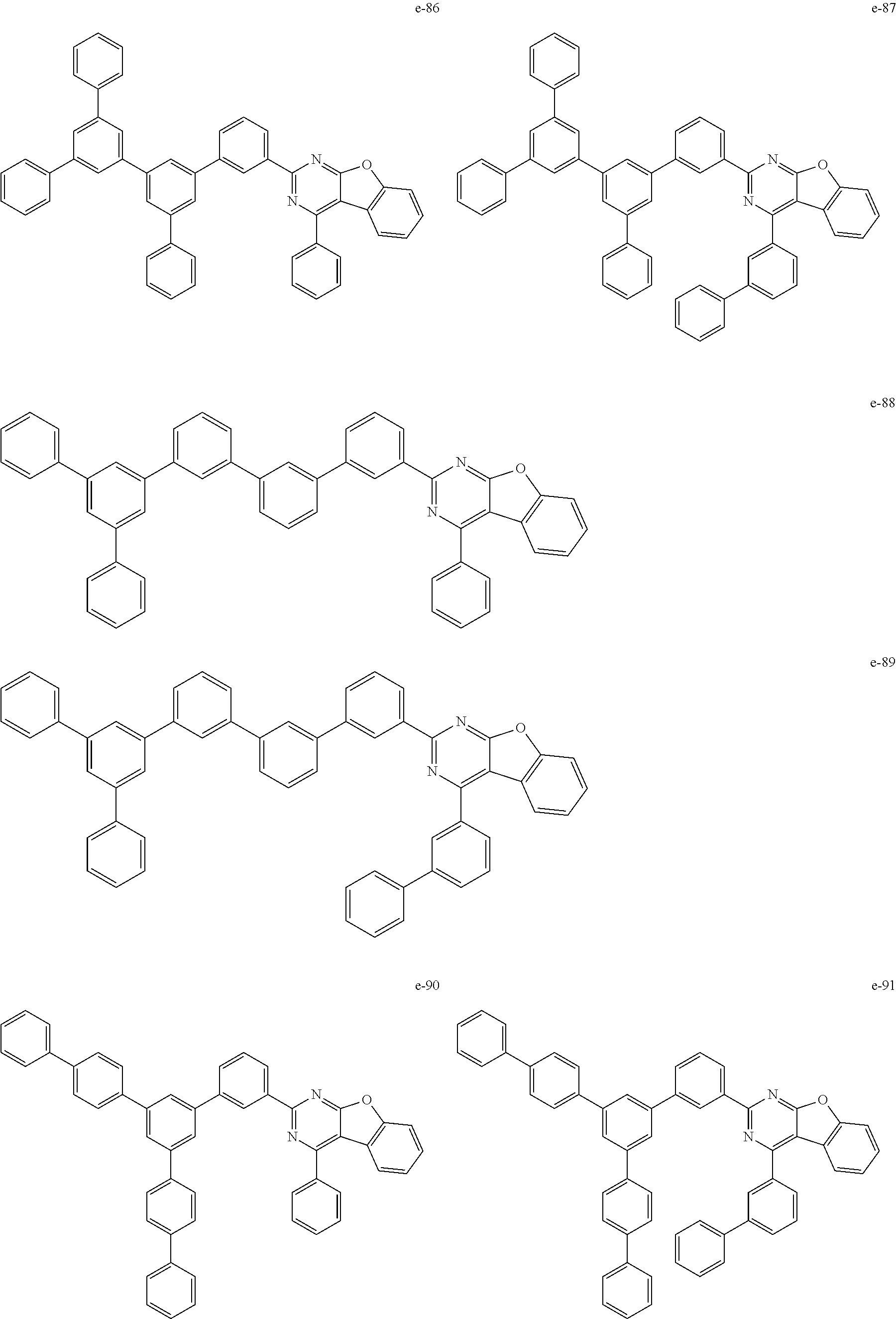

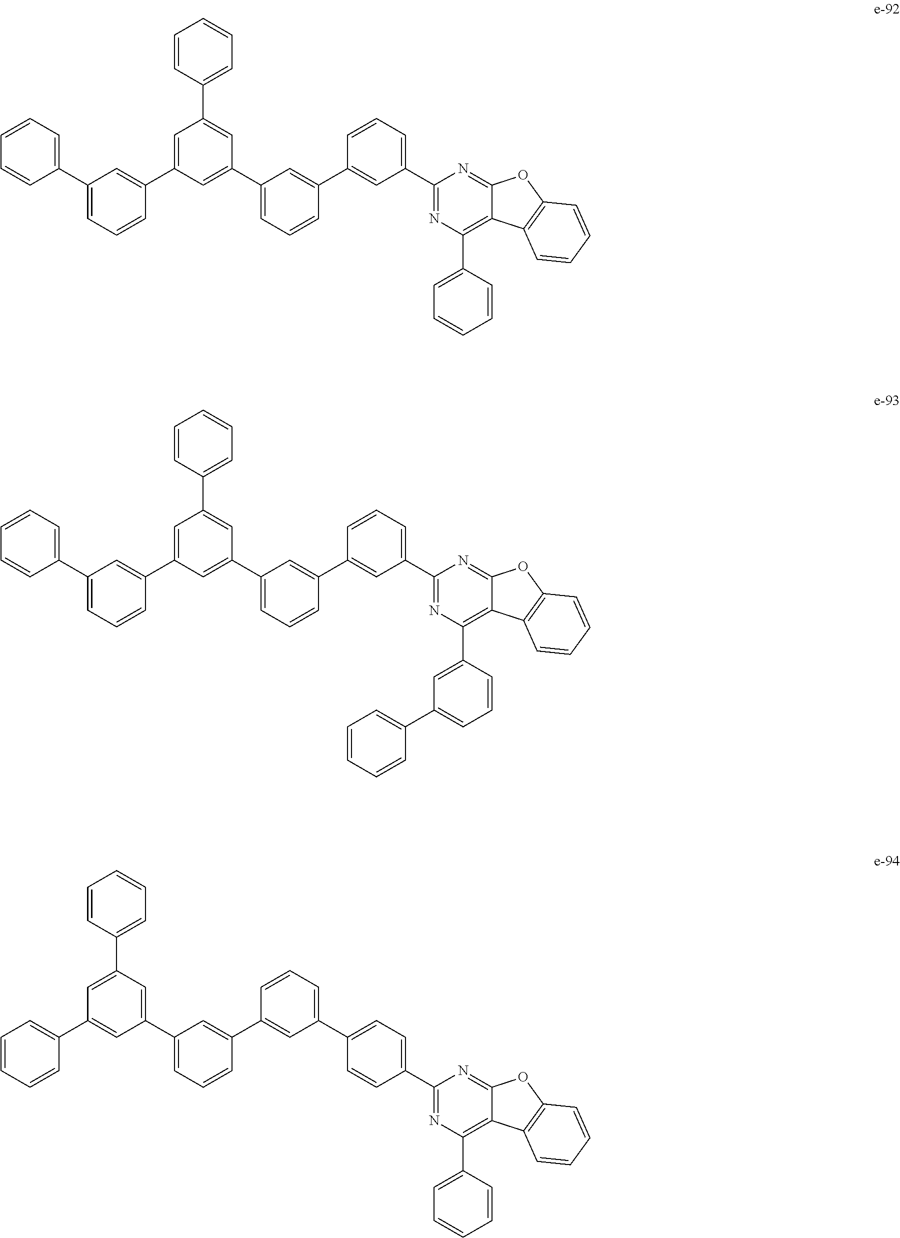 [SMILES: c1ccc(-c2cccc(-c3cc(-c4ccccc4)cc(-c4cccc(-c5cccc(-c6nc(-c7ccccc7)c7c(n6)oc6ccccc67)c5)c4)c3)c2)cc1 ; c1ccc(-c2cccc(-c3cc(-c4ccccc4)cc(-c4cccc(-c5cccc(-c6nc(-c7cccc(-c8ccccc8)c7)c7c(n6)oc6ccccc67)c5)c4)c3)c2)cc1 ; c1ccc(-c2cc(-c3ccccc3)cc(-c3cccc(-c4cccc(-c5ccc(-c6nc(-c7ccccc7)c7c(n6)oc6ccccc67)cc5)c4)c3)c2)cc1]

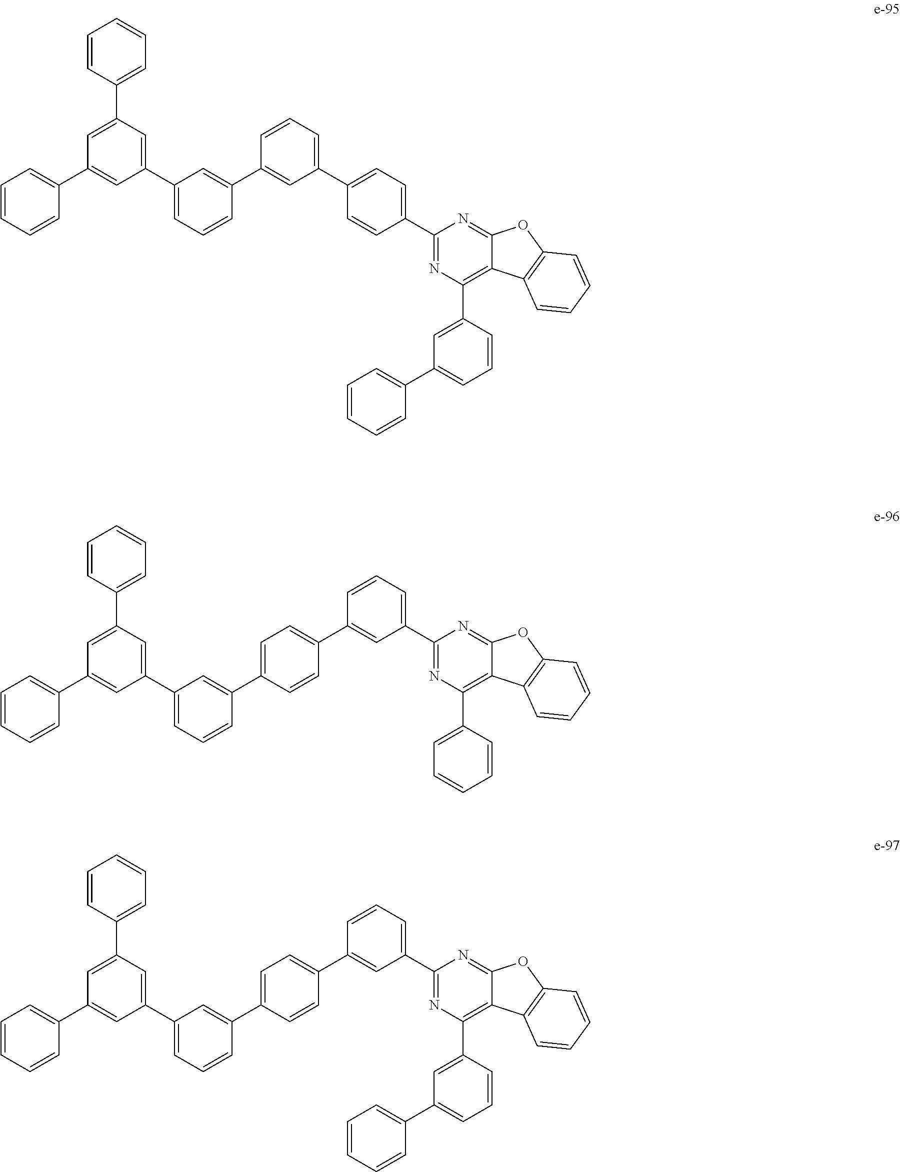 [SMILES: c1ccc(-c2cc(-c3ccccc3)cc(-c3cccc(-c4cccc(-c5ccc(-c6nc(-c7cccc(-c8ccccc8)c7)c7c(n6)oc6ccccc67)cc5)c4)c3)c2)cc1 ; c1ccc(-c2cc(-c3ccccc3)cc(-c3cccc(-c4ccc(-c5cccc(-c6nc(-c7ccccc7)c7c(n6)oc6ccccc67)c5)cc4)c3)c2)cc1 ; c1ccc(-c2cc(-c3ccccc3)cc(-c3cccc(-c4ccc(-c5cccc(-c6nc(-c7cccc(-c8ccccc8)c7)c7c(n6)oc6ccccc67)c5)cc4)c3)c2)cc1]

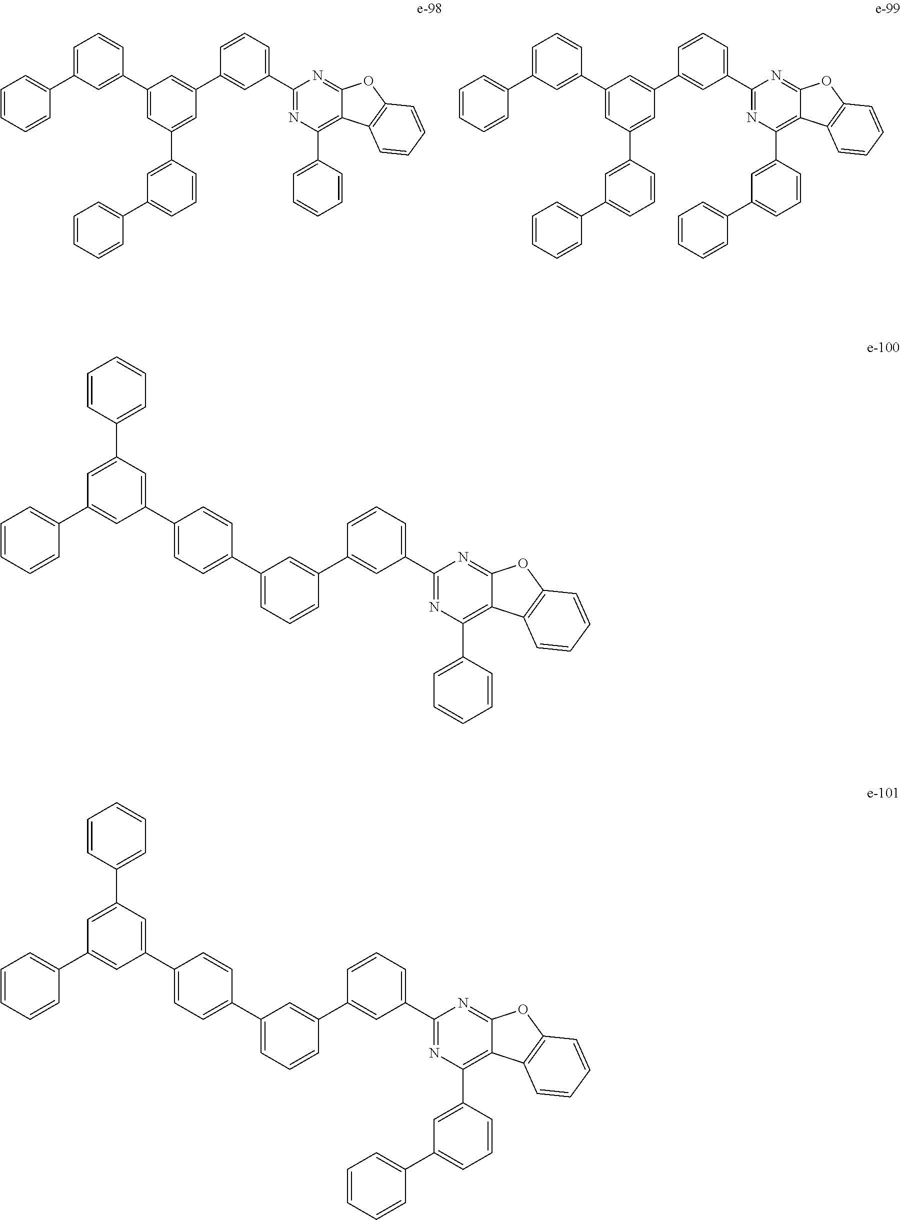

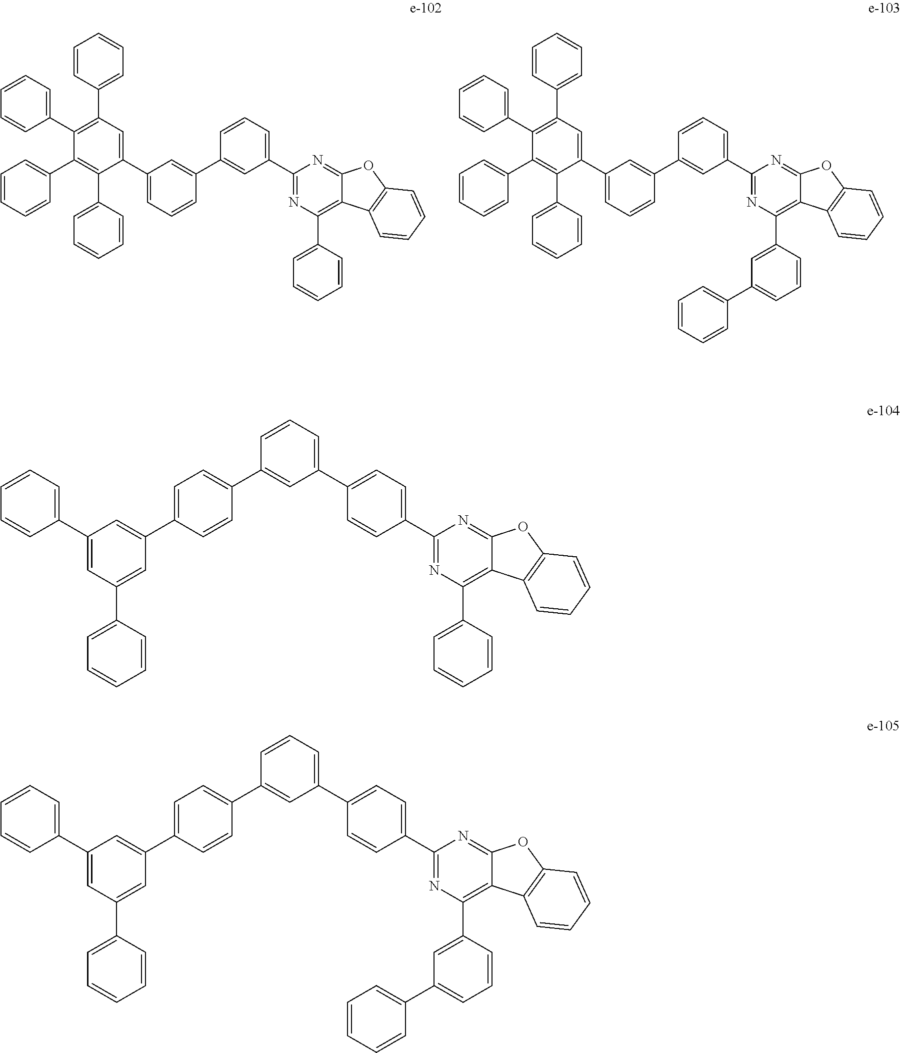

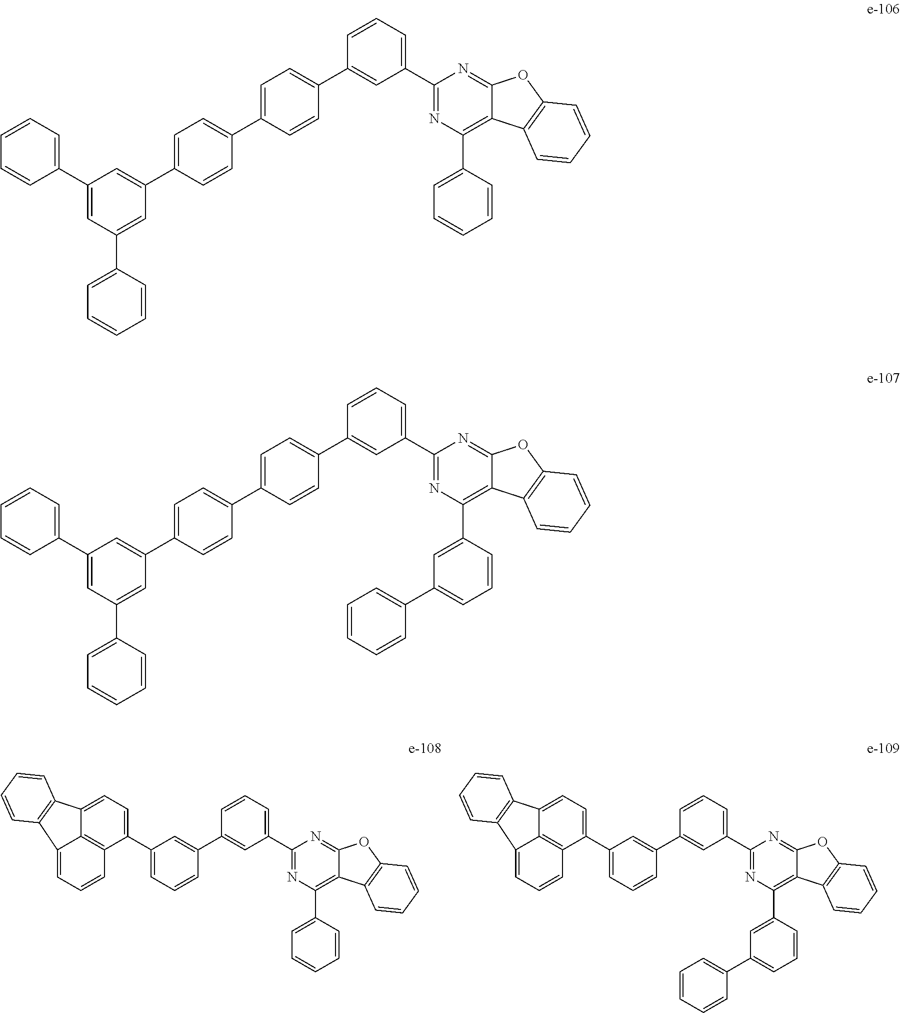 [SMILES: c1ccc(-c2cc(-c3ccccc3)cc(-c3ccc(-c4ccc(-c5cccc(-c6nc(-c7ccccc7)c7c(n6)oc6ccccc67)c5)cc4)cc3)c2)cc1 ; c1ccc(-c2cc(-c3ccccc3)cc(-c3ccc(-c4ccc(-c5cccc(-c6nc(-c7cccc(-c8ccccc8)c7)c7c(n6)oc6ccccc67)c5)cc4)cc3)c2)cc1 ; c1ccc(-c2nc(-c3cccc(-c4cccc(-c5ccc6c7c(cccc57)-c5ccccc5-6)c4)c3)nc3oc4ccccc4c23)cc1 ; c1ccc(-c2cccc(-c3nc(-c4cccc(-c5cccc(-c6ccc7c8c(cccc68)-c6ccccc6-7)c5)c4)nc4oc5ccccc5c34)c2)cc1]

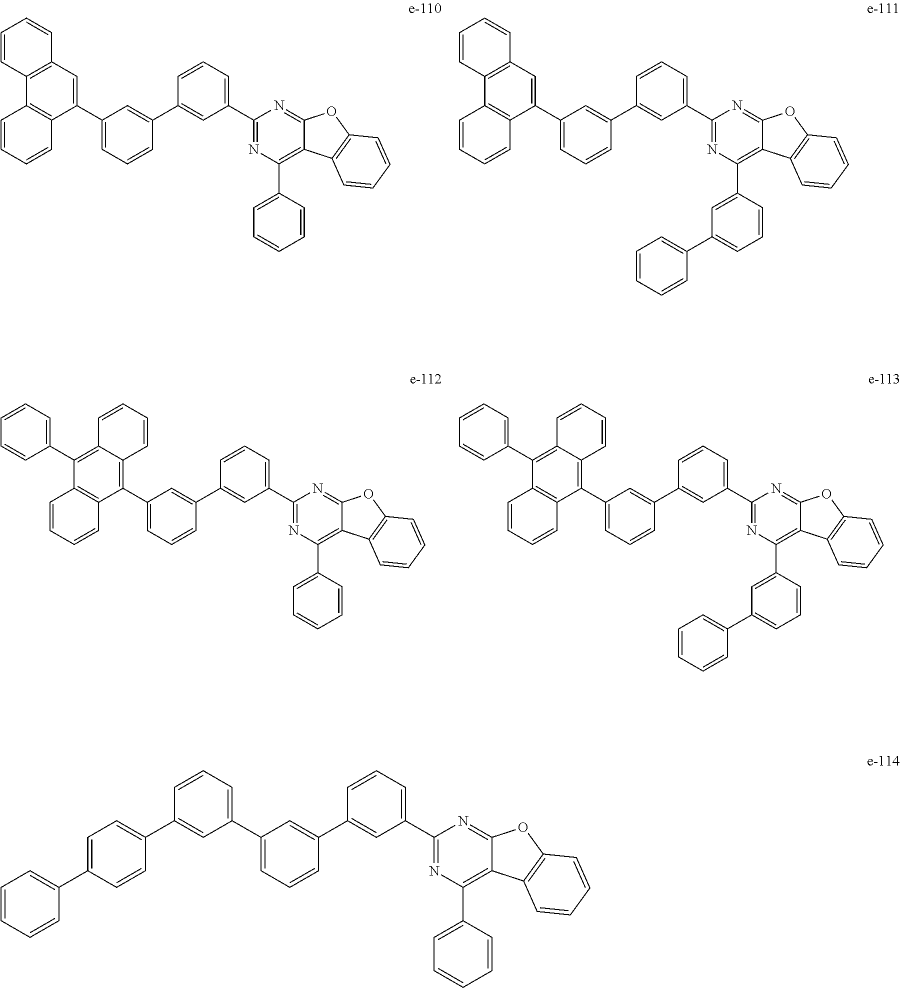 [SMILES: c1ccc(-c2nc(-c3cccc(-c4cccc(-c5cc6ccccc6c6ccccc56)c4)c3)nc3oc4ccccc4c23)cc1 ; c1ccc(-c2cccc(-c3nc(-c4cccc(-c5cccc(-c6cc7ccccc7c7ccccc67)c5)c4)nc4oc5ccccc5c34)c2)cc1 ; c1ccc(-c2c3ccccc3c(-c3cccc(-c4cccc(-c5nc(-c6ccccc6)c6c(n5)oc5ccccc56)c4)c3)c3ccccc23)cc1 ; c1ccc(-c2cccc(-c3nc(-c4cccc(-c5cccc(-c6c7ccccc7c(-c7ccccc7)c7ccccc67)c5)c4)nc4oc5ccccc5c34)c2)cc1 ; c1ccc(-c2ccc(-c3cccc(-c4cccc(-c5cccc(-c6nc(-c7ccccc7)c7c(n6)oc6ccccc67)c5)c4)c3)cc2)cc1]

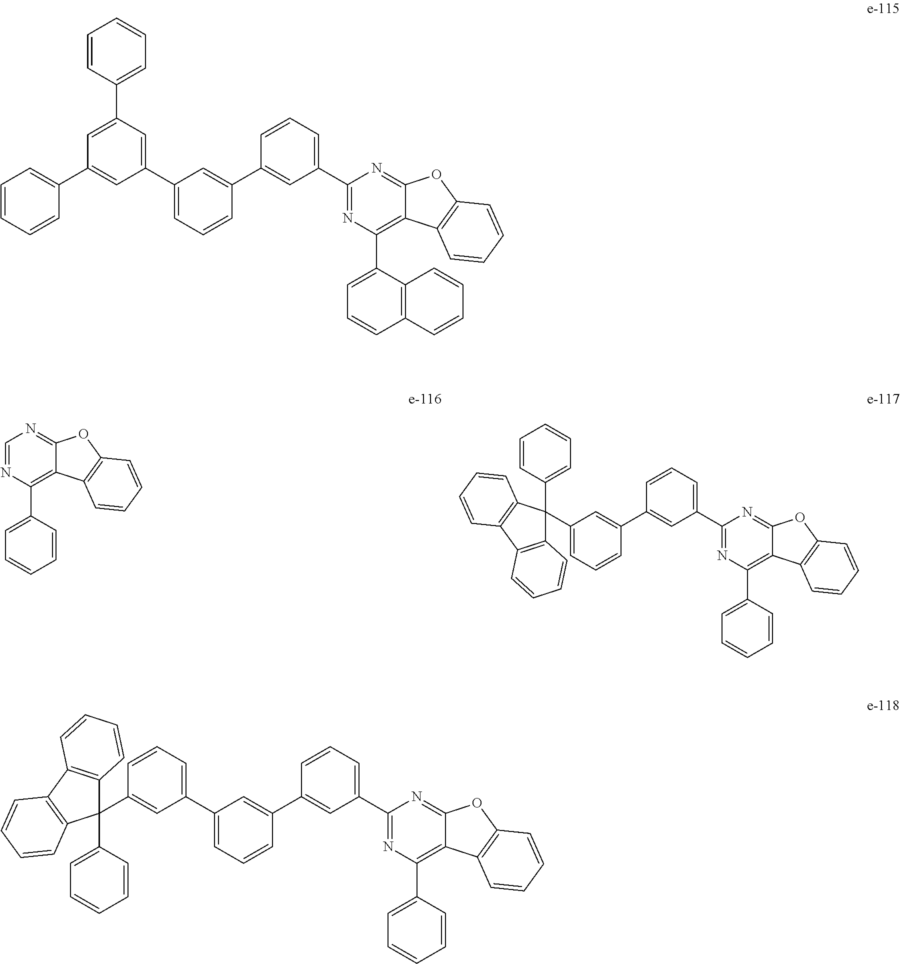 [SMILES: c1ccc(-c2cc(-c3ccccc3)cc(-c3cccc(-c4cccc(-c5nc(-c6cccc7ccccc67)c6c(n5)oc5ccccc56)c4)c3)c2)cc1 ; c1ccc(-c2ncnc3oc4ccccc4c23)cc1 ; c1ccc(-c2nc(-c3cccc(-c4cccc(C5(c6ccccc6)c6ccccc6-c6ccccc65)c4)c3)nc3oc4ccccc4c23)cc1 ; c1ccc(-c2nc(-c3cccc(-c4cccc(-c5cccc(C6(c7ccccc7)c7ccccc7-c7ccccc76)c5)c4)c3)nc3oc4ccccc4c23)cc1]

Group of X₁=S in Formula 1-2

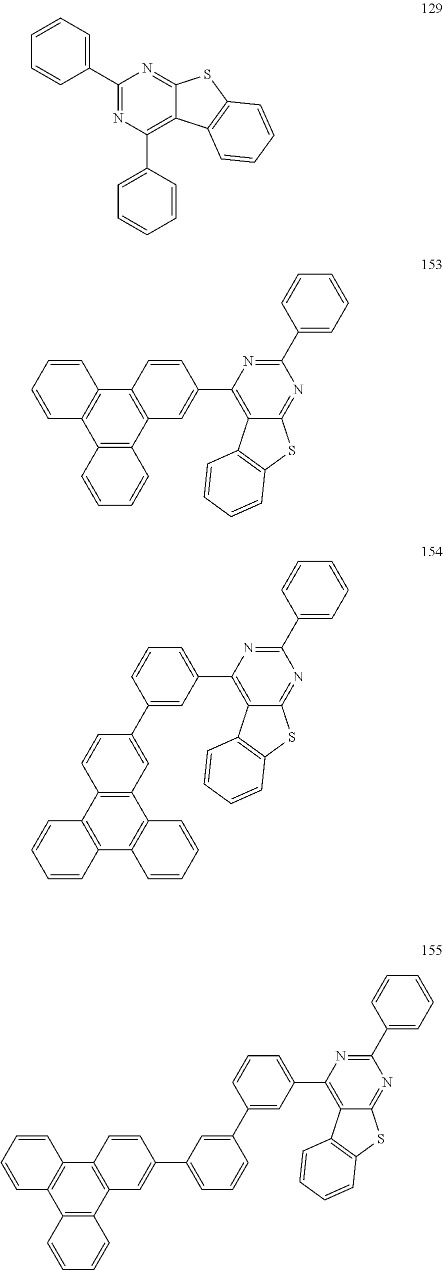 [SMILES: c1ccc(-c2nc(-c3ccccc3)c3c(n2)sc2ccccc23)cc1 ; c1ccc(-c2nc(-c3ccc4c5ccccc5c5ccccc5c4c3)c3c(n2)sc2ccccc23)cc1 ; c1ccc(-c2nc(-c3cccc(-c4ccc5c6ccccc6c6ccccc6c5c4)c3)c3c(n2)sc2ccccc23)cc1 ; c1ccc(-c2nc(-c3cccc(-c4cccc(-c5ccc6c7ccccc7c7ccccc7c6c5)c4)c3)c3c(n2)sc2ccccc23)cc1]

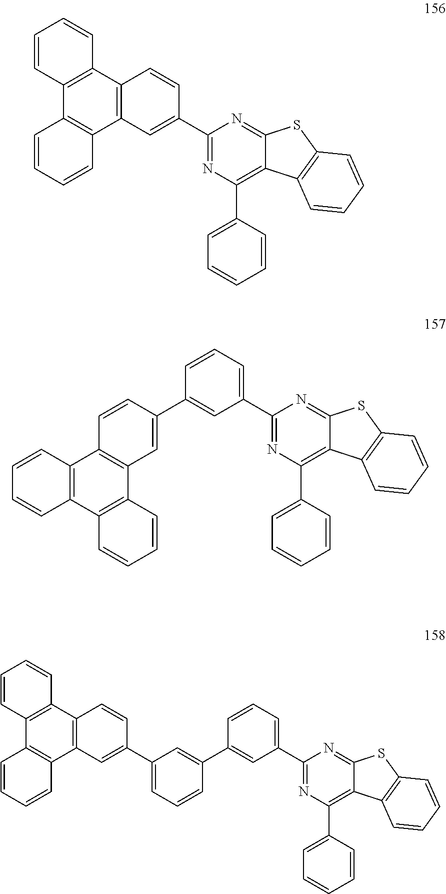 [SMILES: c1ccc(-c2nc(-c3ccc4c5ccccc5c5ccccc5c4c3)nc3sc4ccccc4c23)cc1 ; c1ccc(-c2nc(-c3cccc(-c4ccc5c6ccccc6c6ccccc6c5c4)c3)nc3sc4ccccc4c23)cc1 ; c1ccc(-c2nc(-c3cccc(-c4cccc(-c5ccc6c7ccccc7c7ccccc7c6c5)c4)c3)nc3sc4ccccc4c23)cc1]

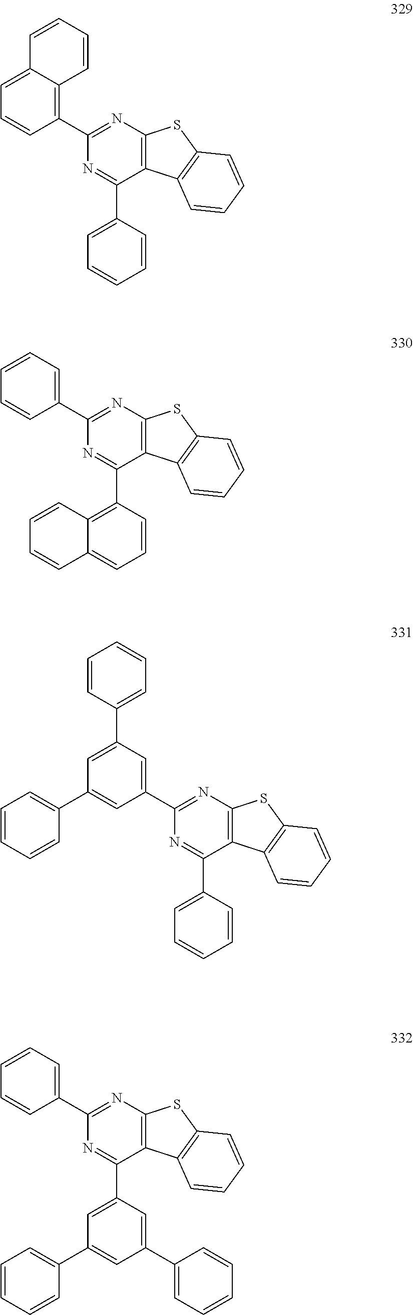 [SMILES: c1ccc(-c2nc(-c3cccc4ccccc34)nc3sc4ccccc4c23)cc1 ; c1ccc(-c2nc(-c3cccc4ccccc34)c3c(n2)sc2ccccc23)cc1 ; c1ccc(-c2cc(-c3ccccc3)cc(-c3nc(-c4ccccc4)c4c(n3)sc3ccccc34)c2)cc1 ; c1ccc(-c2cc(-c3ccccc3)cc(-c3nc(-c4ccccc4)nc4sc5ccccc5c34)c2)cc1]

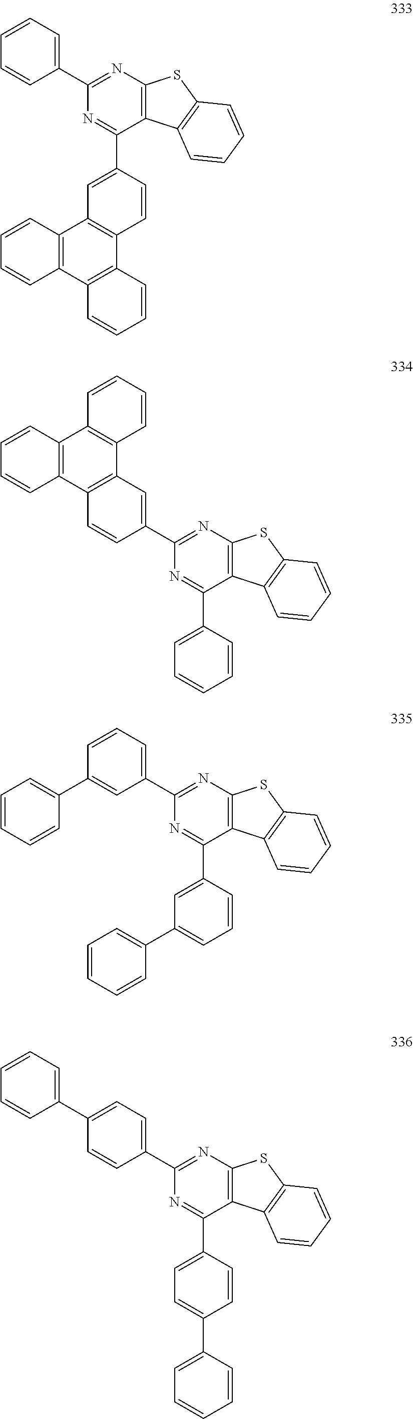

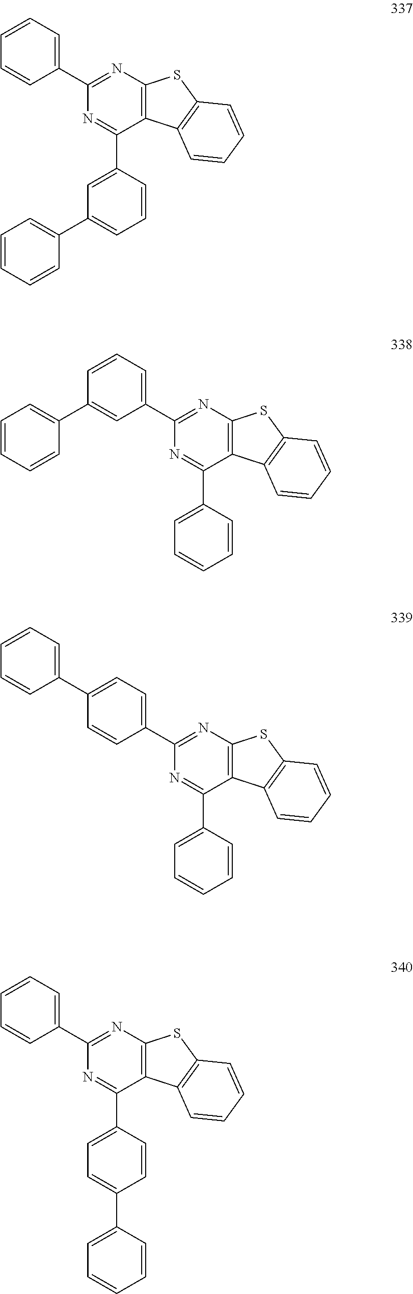 [SMILES: c1ccc(-c2cccc(-c3nc(-c4ccccc4)nc4sc5ccccc5c34)c2)cc1 ; c1ccc(-c2cccc(-c3nc(-c4ccccc4)c4c(n3)sc3ccccc34)c2)cc1 ; c1ccc(-c2ccc(-c3nc(-c4ccccc4)c4c(n3)sc3ccccc34)cc2)cc1 ; c1ccc(-c2ccc(-c3nc(-c4ccccc4)nc4sc5ccccc5c34)cc2)cc1]

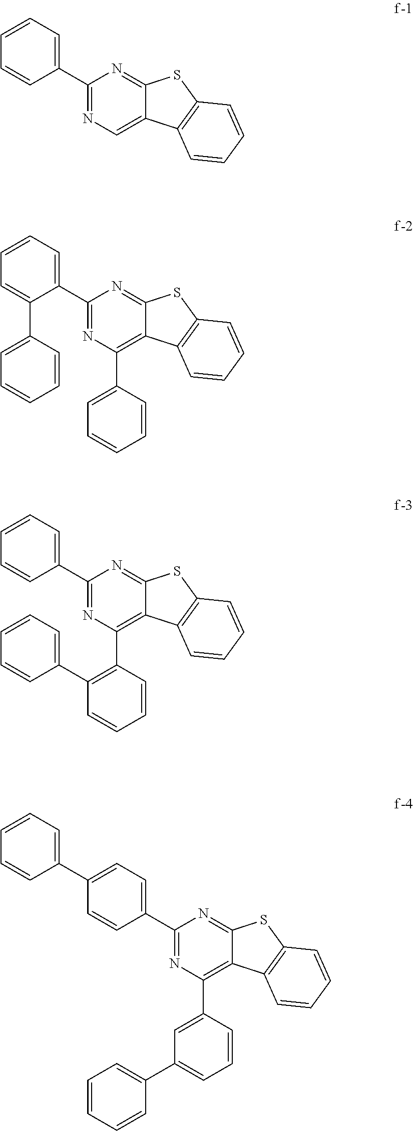 [SMILES: c1ccc(-c2ncc3c(n2)sc2ccccc23)cc1 ; c1ccc(-c2ccccc2-c2nc(-c3ccccc3)c3c(n2)sc2ccccc23)cc1 ; c1ccc(-c2nc(-c3ccccc3-c3ccccc3)c3c(n2)sc2ccccc23)cc1 ; c1ccc(-c2ccc(-c3nc(-c4cccc(-c5ccccc5)c4)c4c(n3)sc3ccccc34)cc2)cc1]

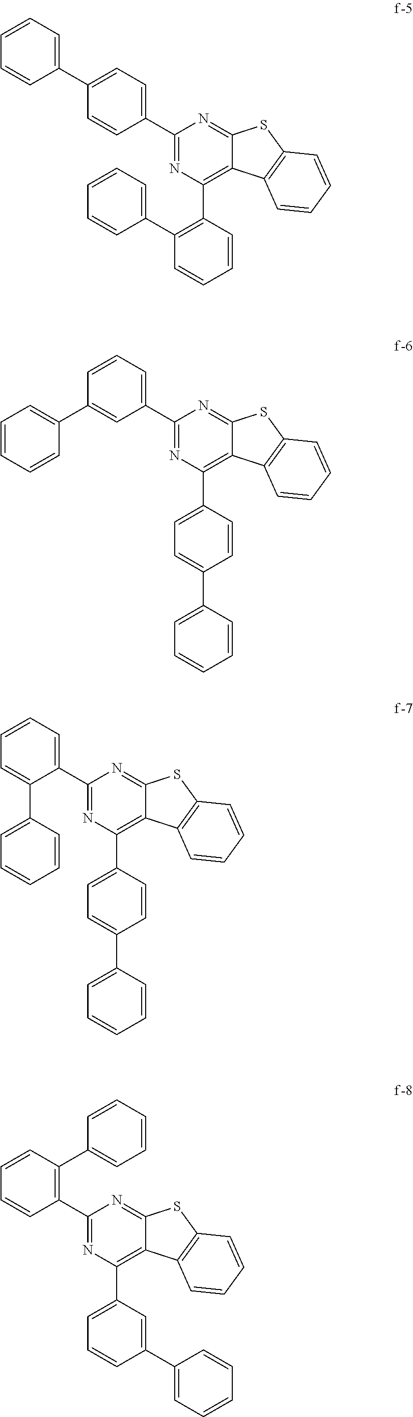 [SMILES: c1ccc(-c2ccc(-c3nc(-c4ccccc4-c4ccccc4)c4c(n3)sc3ccccc34)cc2)cc1 ; c1ccc(-c2ccc(-c3nc(-c4cccc(-c5ccccc5)c4)nc4sc5ccccc5c34)cc2)cc1 ; c1ccc(-c2ccc(-c3nc(-c4ccccc4-c4ccccc4)nc4sc5ccccc5c34)cc2)cc1 ; c1ccc(-c2cccc(-c3nc(-c4ccccc4-c4ccccc4)nc4sc5ccccc5c34)c2)cc1]

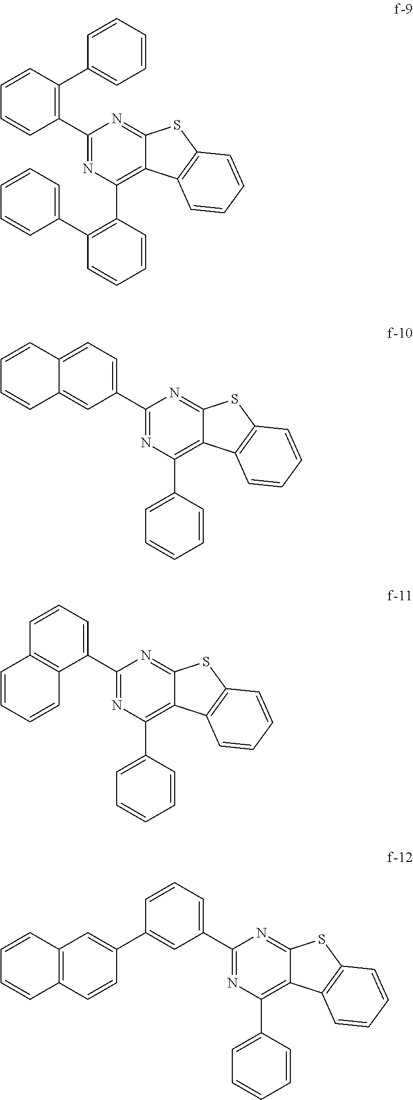 [SMILES: c1ccc(-c2ccccc2-c2nc(-c3ccccc3-c3ccccc3)c3c(n2)sc2ccccc23)cc1 ; c1ccc(-c2nc(-c3ccc4ccccc4c3)nc3sc4ccccc4c23)cc1 ; c1ccc(-c2nc(-c3cccc4ccccc34)nc3sc4ccccc4c23)cc1 ; c1ccc(-c2nc(-c3cccc(-c4ccc5ccccc5c4)c3)nc3sc4ccccc4c23)cc1]

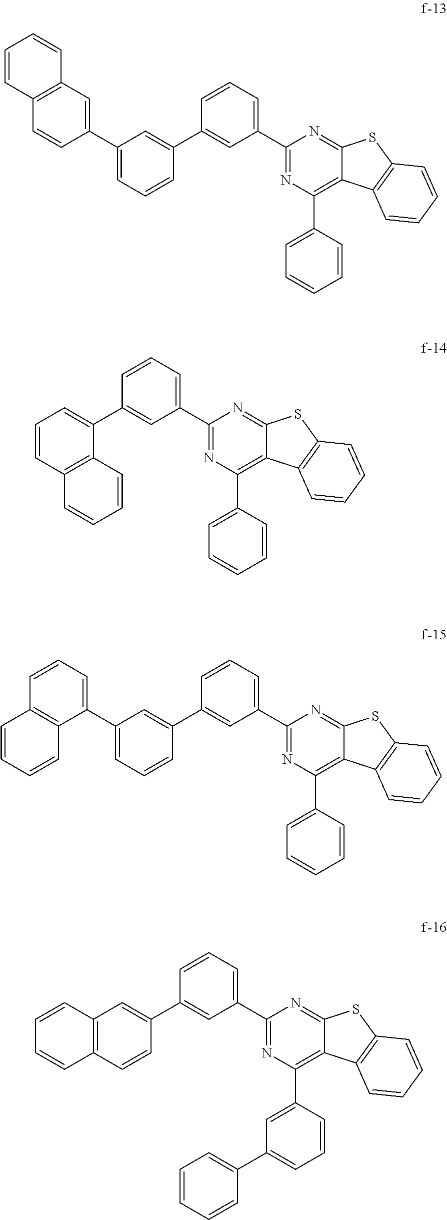 [SMILES: c1ccc(-c2nc(-c3cccc(-c4cccc(-c5ccc6ccccc6c5)c4)c3)nc3sc4ccccc4c23)cc1 ; c1ccc(-c2nc(-c3cccc(-c4cccc5ccccc45)c3)nc3sc4ccccc4c23)cc1 ; c1ccc(-c2nc(-c3cccc(-c4cccc(-c5cccc6ccccc56)c4)c3)nc3sc4ccccc4c23)cc1 ; c1ccc(-c2cccc(-c3nc(-c4cccc(-c5ccc6ccccc6c5)c4)nc4sc5ccccc5c34)c2)cc1]

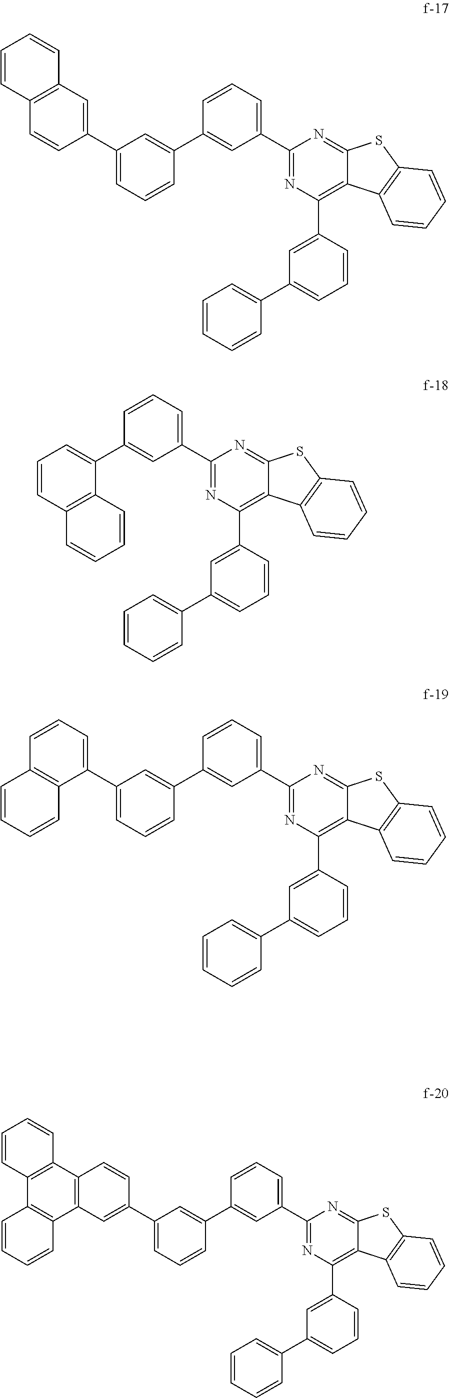 [SMILES: c1ccc(-c2cccc(-c3nc(-c4cccc(-c5cccc(-c6ccc7ccccc7c6)c5)c4)nc4sc5ccccc5c34)c2)cc1 ; c1ccc(-c2cccc(-c3nc(-c4cccc(-c5cccc6ccccc56)c4)nc4sc5ccccc5c34)c2)cc1 ; c1ccc(-c2cccc(-c3nc(-c4cccc(-c5cccc(-c6cccc7ccccc67)c5)c4)nc4sc5ccccc5c34)c2)cc1 ; c1ccc(-c2cccc(-c3nc(-c4cccc(-c5cccc(-c6ccc7c8ccccc8c8ccccc8c7c6)c5)c4)nc4sc5ccccc5c34)c2)cc1]

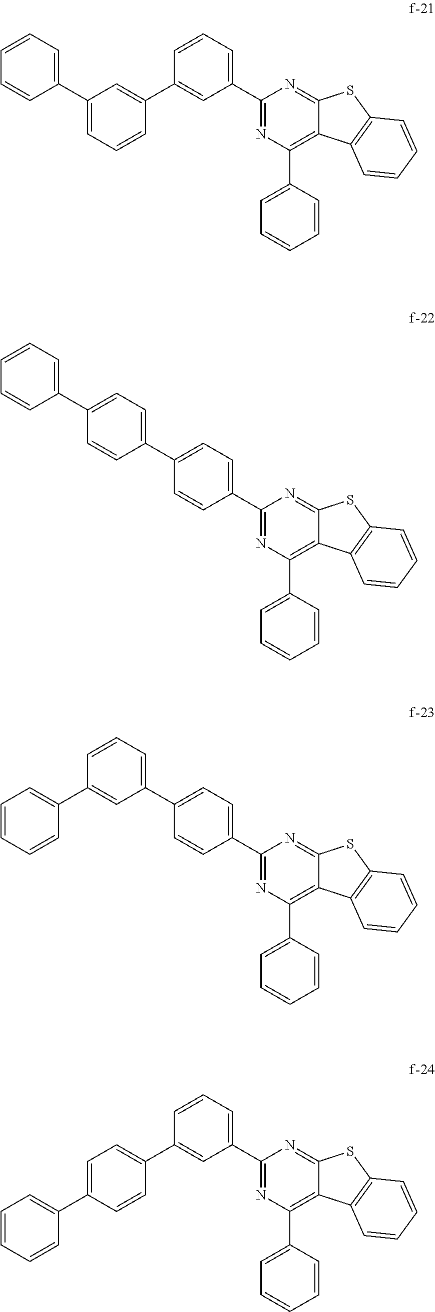 [SMILES: c1ccc(-c2cccc(-c3cccc(-c4nc(-c5ccccc5)c5c(n4)sc4ccccc45)c3)c2)cc1 ; c1ccc(-c2ccc(-c3ccc(-c4nc(-c5ccccc5)c5c(n4)sc4ccccc45)cc3)cc2)cc1 ; c1ccc(-c2cccc(-c3ccc(-c4nc(-c5ccccc5)c5c(n4)sc4ccccc45)cc3)c2)cc1 ; c1ccc(-c2ccc(-c3cccc(-c4nc(-c5ccccc5)c5c(n4)sc4ccccc45)c3)cc2)cc1]

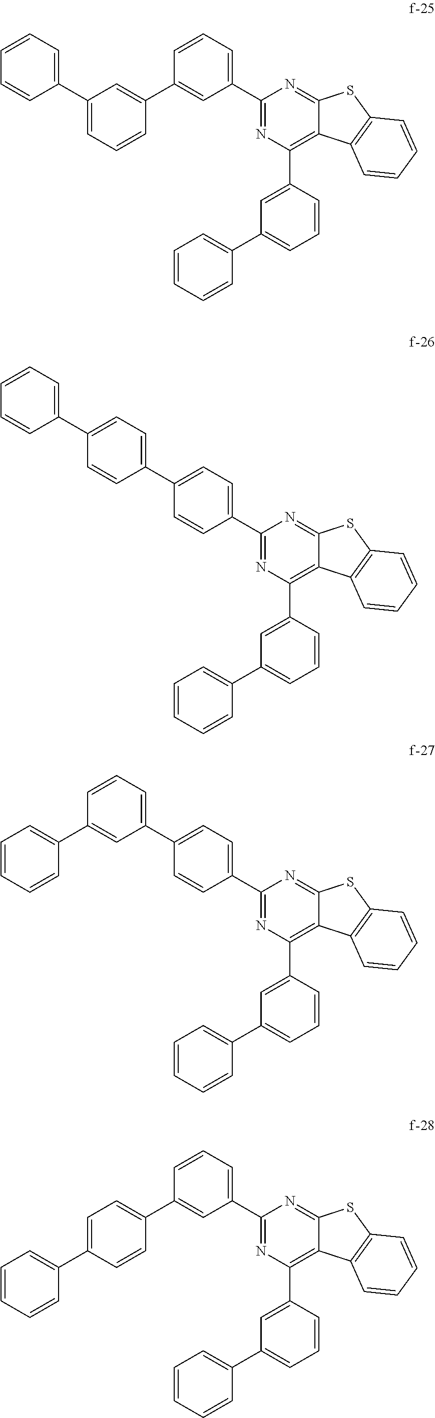 [SMILES: c1ccc(-c2cccc(-c3cccc(-c4nc(-c5cccc(-c6ccccc6)c5)c5c(n4)sc4ccccc45)c3)c2)cc1 ; c1ccc(-c2ccc(-c3ccc(-c4nc(-c5cccc(-c6ccccc6)c5)c5c(n4)sc4ccccc45)cc3)cc2)cc1 ; c1ccc(-c2cccc(-c3ccc(-c4nc(-c5cccc(-c6ccccc6)c5)c5c(n4)sc4ccccc45)cc3)c2)cc1 ; c1ccc(-c2ccc(-c3cccc(-c4nc(-c5cccc(-c6ccccc6)c5)c5c(n4)sc4ccccc45)c3)cc2)cc1]

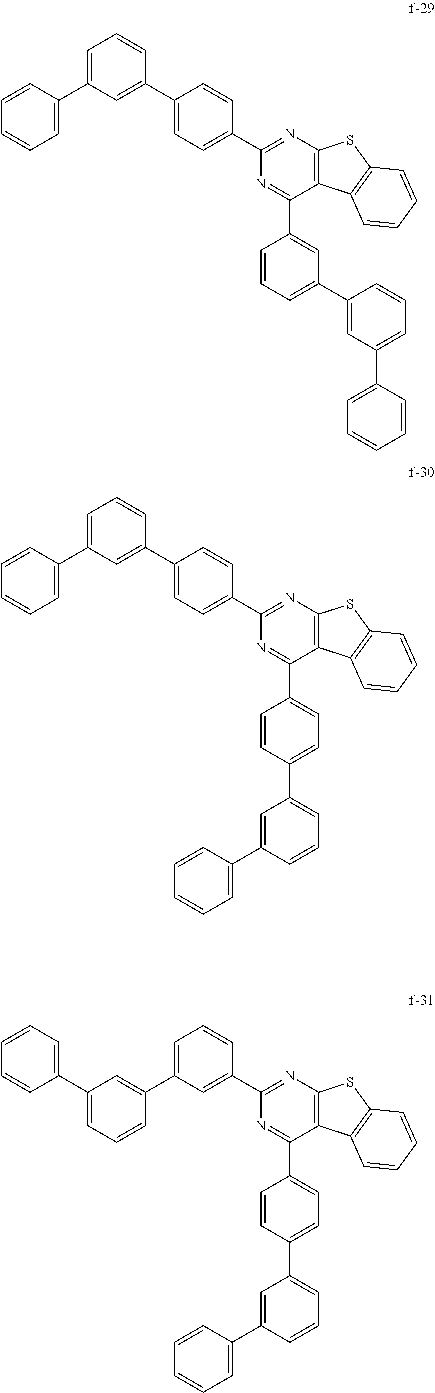 [SMILES: c1ccc(-c2cccc(-c3ccc(-c4nc(-c5cccc(-c6cccc(-c7ccccc7)c6)c5)c5c(n4)sc4ccccc45)cc3)c2)cc1 ; c1ccc(-c2cccc(-c3ccc(-c4nc(-c5ccc(-c6cccc(-c7ccccc7)c6)cc5)c5c(n4)sc4ccccc45)cc3)c2)cc1 ; c1ccc(-c2cccc(-c3ccc(-c4nc(-c5cccc(-c6cccc(-c7ccccc7)c6)c5)nc5sc6ccccc6c45)cc3)c2)cc1]

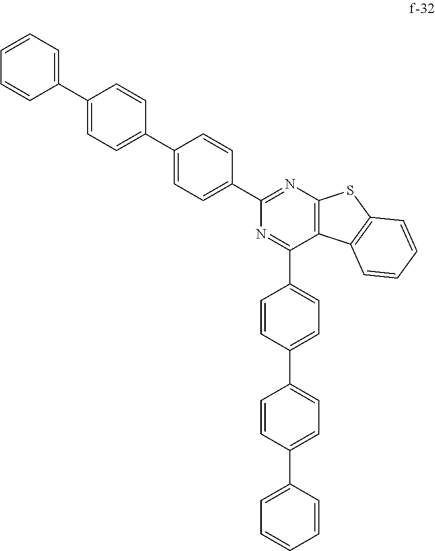 [SMILES: c1ccc(-c2ccc(-c3ccc(-c4nc(-c5ccc(-c6ccc(-c7ccccc7)cc6)cc5)c5c(n4)sc4ccccc45)cc3)cc2)cc1]

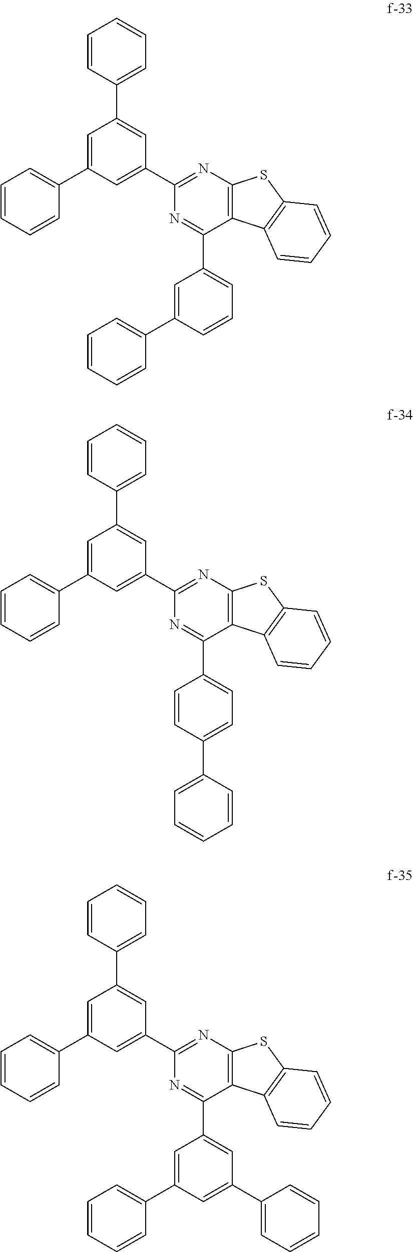

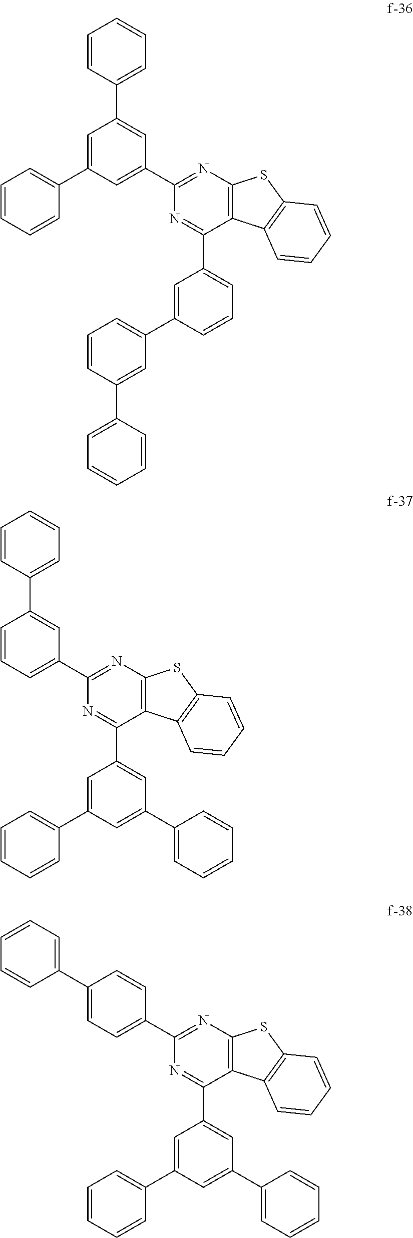

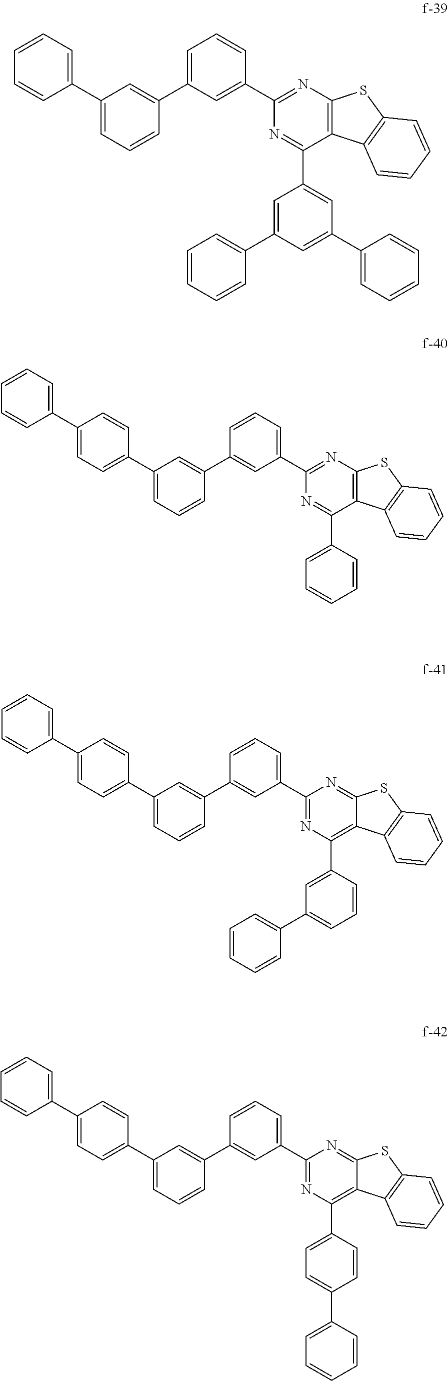 [SMILES: c1ccc(-c2cccc(-c3cccc(-c4nc(-c5cc(-c6ccccc6)cc(-c6ccccc6)c5)c5c(n4)sc4ccccc45)c3)c2)cc1 ; c1ccc(-c2ccc(-c3cccc(-c4cccc(-c5nc(-c6ccccc6)c6c(n5)sc5ccccc56)c4)c3)cc2)cc1 ; c1ccc(-c2ccc(-c3cccc(-c4cccc(-c5nc(-c6cccc(-c7ccccc7)c6)c6c(n5)sc5ccccc56)c4)c3)cc2)cc1 ; c1ccc(-c2ccc(-c3cccc(-c4cccc(-c5nc(-c6ccc(-c7ccccc7)cc6)c6c(n5)sc5ccccc56)c4)c3)cc2)cc1]

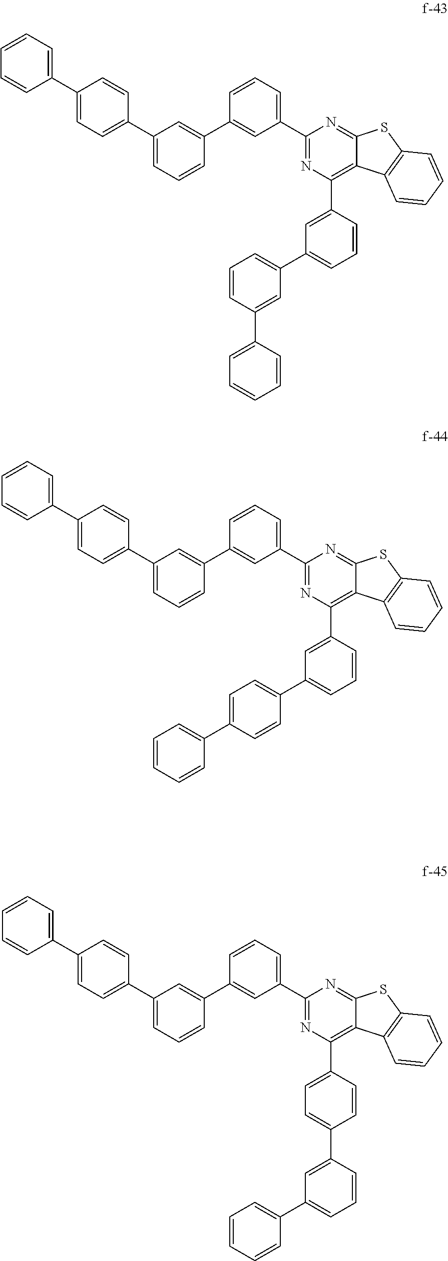 [SMILES: c1ccc(-c2ccc(-c3cccc(-c4cccc(-c5nc(-c6cccc(-c7cccc(-c8ccccc8)c7)c6)c6c(n5)sc5ccccc56)c4)c3)cc2)cc1 ; c1ccc(-c2ccc(-c3cccc(-c4cccc(-c5nc(-c6cccc(-c7ccc(-c8ccccc8)cc7)c6)c6c(n5)sc5ccccc56)c4)c3)cc2)cc1 ; c1ccc(-c2ccc(-c3cccc(-c4cccc(-c5nc(-c6ccc(-c7cccc(-c8ccccc8)c7)cc6)c6c(n5)sc5ccccc56)c4)c3)cc2)cc1]

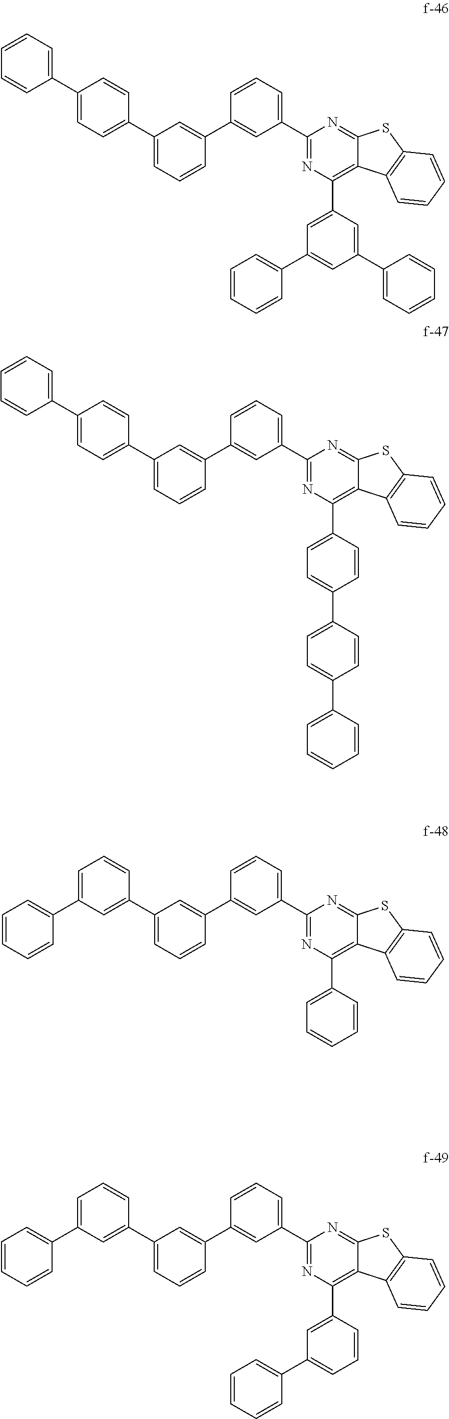 [SMILES: c1ccc(-c2ccc(-c3cccc(-c4cccc(-c5nc(-c6cc(-c7ccccc7)cc(-c7ccccc7)c6)c6c(n5)sc5ccccc56)c4)c3)cc2)cc1 ; c1ccc(-c2ccc(-c3ccc(-c4nc(-c5cccc(-c6cccc(-c7ccc(-c8ccccc8)cc7)c6)c5)nc5sc6ccccc6c45)cc3)cc2)cc1 ; c1ccc(-c2cccc(-c3cccc(-c4cccc(-c5nc(-c6ccccc6)c6c(n5)sc5ccccc56)c4)c3)c2)cc1 ; c1ccc(-c2cccc(-c3cccc(-c4cccc(-c5nc(-c6cccc(-c7ccccc7)c6)c6c(n5)sc5ccccc56)c4)c3)c2)cc1]

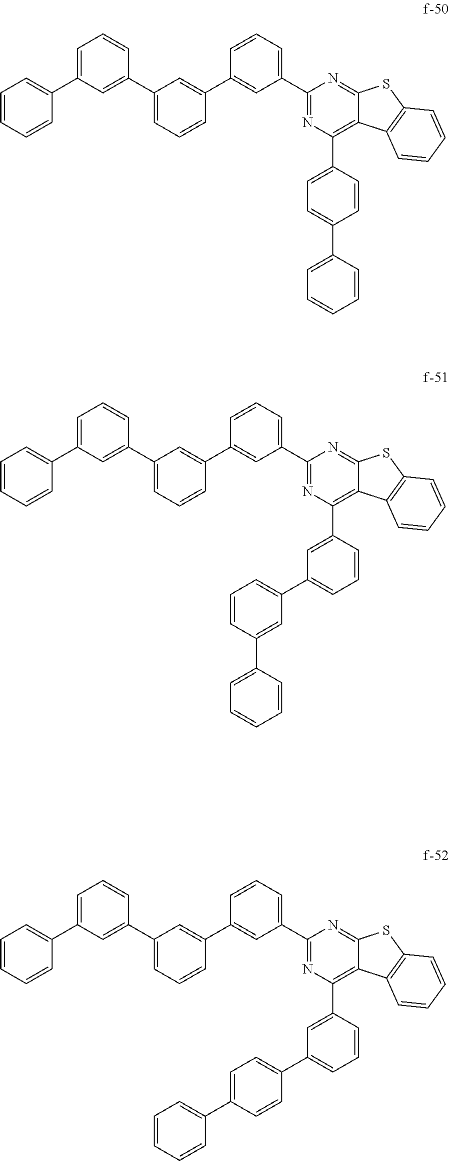 [SMILES: c1ccc(-c2ccc(-c3nc(-c4cccc(-c5cccc(-c6cccc(-c7ccccc7)c6)c5)c4)nc4sc5ccccc5c34)cc2)cc1 ; c1ccc(-c2cccc(-c3cccc(-c4cccc(-c5nc(-c6cccc(-c7cccc(-c8ccccc8)c7)c6)c6c(n5)sc5ccccc56)c4)c3)c2)cc1 ; c1ccc(-c2ccc(-c3cccc(-c4nc(-c5cccc(-c6cccc(-c7cccc(-c8ccccc8)c7)c6)c5)nc5sc6ccccc6c45)c3)cc2)cc1]

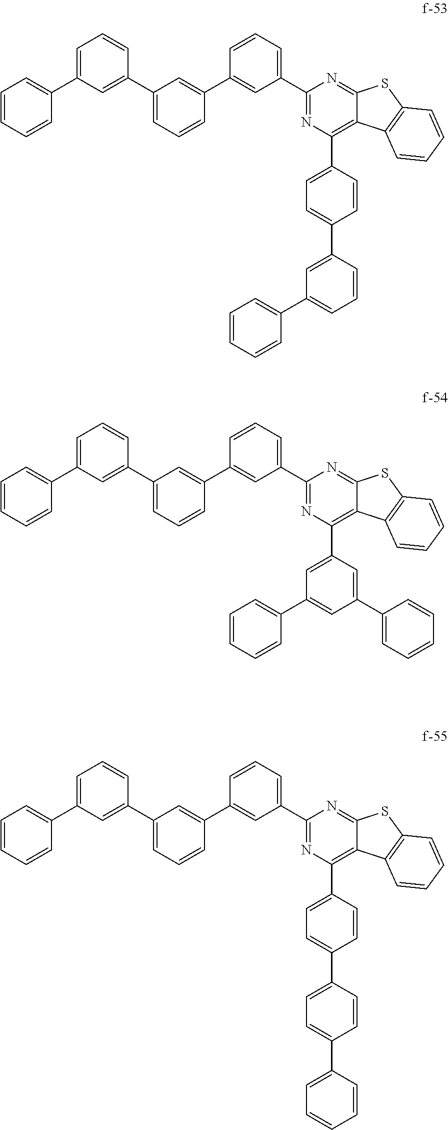 [SMILES: c1ccc(-c2cccc(-c3ccc(-c4nc(-c5cccc(-c6cccc(-c7cccc(-c8ccccc8)c7)c6)c5)nc5sc6ccccc6c45)cc3)c2)cc1 ; c1ccc(-c2cccc(-c3cccc(-c4cccc(-c5nc(-c6cc(-c7ccccc7)cc(-c7ccccc7)c6)c6c(n5)sc5ccccc56)c4)c3)c2)cc1 ; c1ccc(-c2ccc(-c3ccc(-c4nc(-c5cccc(-c6cccc(-c7cccc(-c8ccccc8)c7)c6)c5)nc5sc6ccccc6c45)cc3)cc2)cc1]

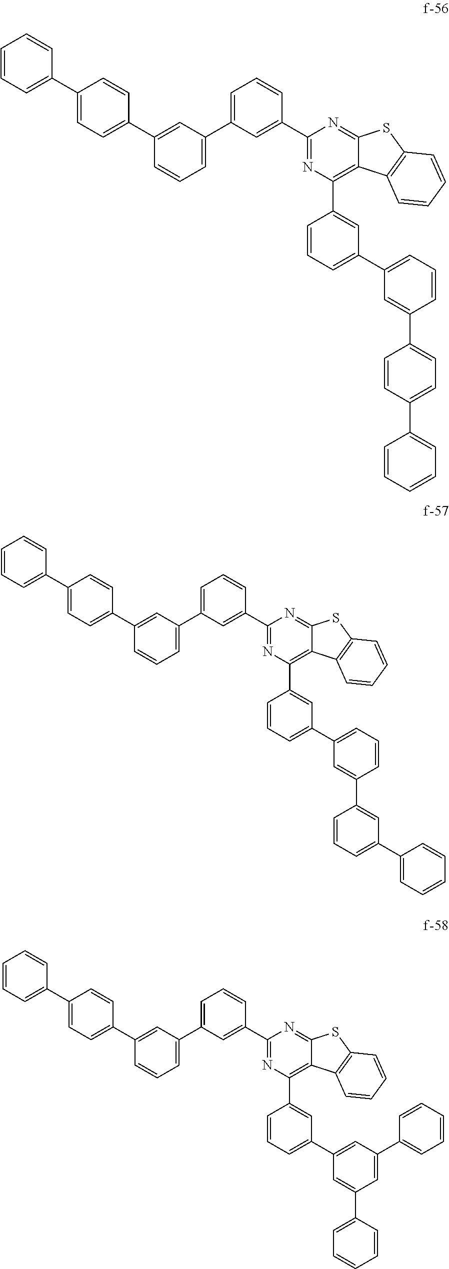 [SMILES: c1ccc(-c2ccc(-c3cccc(-c4cccc(-c5nc(-c6cccc(-c7cccc(-c8ccc(-c9ccccc9)cc8)c7)c6)c6c(n5)sc5ccccc56)c4)c3)cc2)cc1 ; c1ccc(-c2ccc(-c3cccc(-c4cccc(-c5nc(-c6cccc(-c7cccc(-c8cccc(-c9ccccc9)c8)c7)c6)c6c(n5)sc5ccccc56)c4)c3)cc2)cc1 ; c1ccc(-c2ccc(-c3cccc(-c4cccc(-c5nc(-c6cccc(-c7cc(-c8ccccc8)cc(-c8ccccc8)c7)c6)c6c(n5)sc5ccccc56)c4)c3)cc2)cc1]

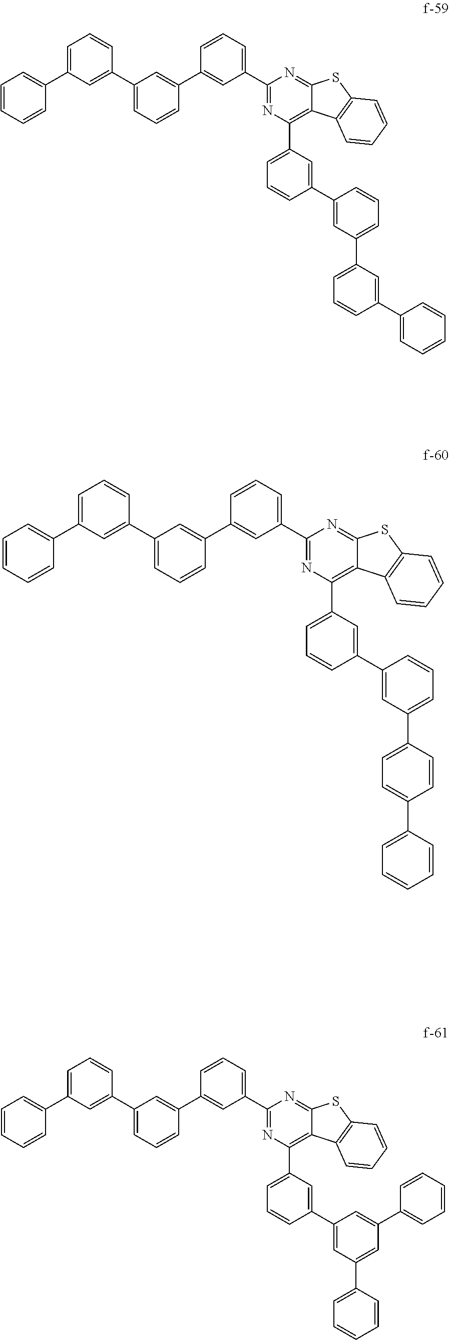 [SMILES: c1ccc(-c2cccc(-c3cccc(-c4cccc(-c5nc(-c6cccc(-c7cccc(-c8cccc(-c9ccccc9)c8)c7)c6)c6c(n5)sc5ccccc56)c4)c3)c2)cc1 ; c1ccc(-c2ccc(-c3cccc(-c4cccc(-c5nc(-c6cccc(-c7cccc(-c8cccc(-c9ccccc9)c8)c7)c6)nc6sc7ccccc7c56)c4)c3)cc2)cc1 ; c1ccc(-c2cccc(-c3cccc(-c4cccc(-c5nc(-c6cccc(-c7cc(-c8ccccc8)cc(-c8ccccc8)c7)c6)c6c(n5)sc5ccccc56)c4)c3)c2)cc1]

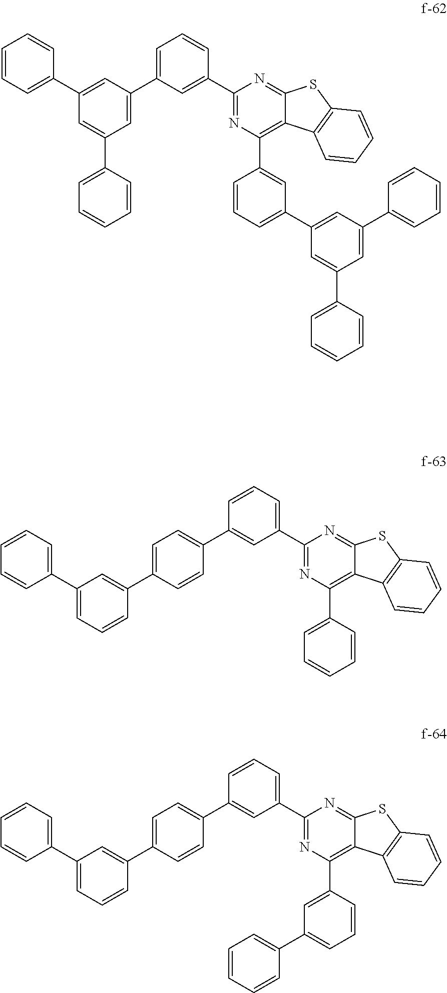 [SMILES: c1ccc(-c2cc(-c3ccccc3)cc(-c3cccc(-c4nc(-c5cccc(-c6cc(-c7ccccc7)cc(-c7ccccc7)c6)c5)c5c(n4)sc4ccccc45)c3)c2)cc1 ; c1ccc(-c2cccc(-c3ccc(-c4cccc(-c5nc(-c6ccccc6)c6c(n5)sc5ccccc56)c4)cc3)c2)cc1 ; c1ccc(-c2cccc(-c3ccc(-c4cccc(-c5nc(-c6cccc(-c7ccccc7)c6)c6c(n5)sc5ccccc56)c4)cc3)c2)cc1]

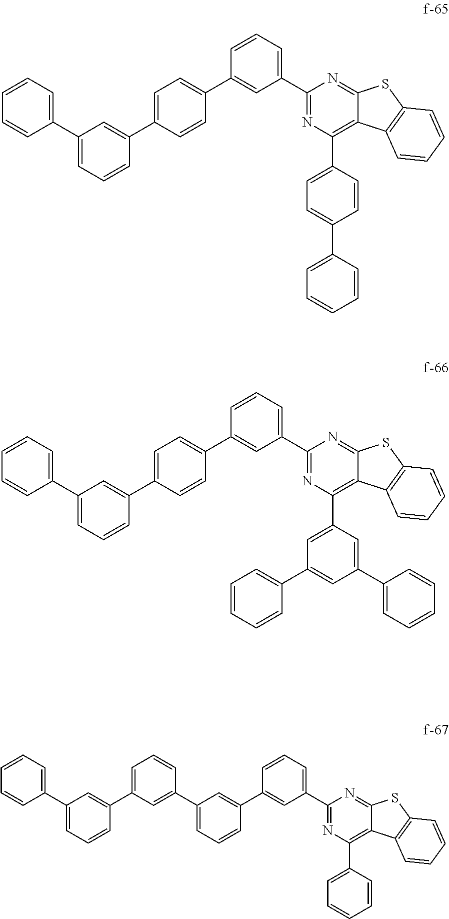 [SMILES: c1ccc(-c2ccc(-c3nc(-c4cccc(-c5ccc(-c6cccc(-c7ccccc7)c6)cc5)c4)nc4sc5ccccc5c34)cc2)cc1 ; c1ccc(-c2cccc(-c3ccc(-c4cccc(-c5nc(-c6cc(-c7ccccc7)cc(-c7ccccc7)c6)c6c(n5)sc5ccccc56)c4)cc3)c2)cc1 ; c1ccc(-c2cccc(-c3cccc(-c4cccc(-c5cccc(-c6nc(-c7ccccc7)c7c(n6)sc6ccccc67)c5)c4)c3)c2)cc1]

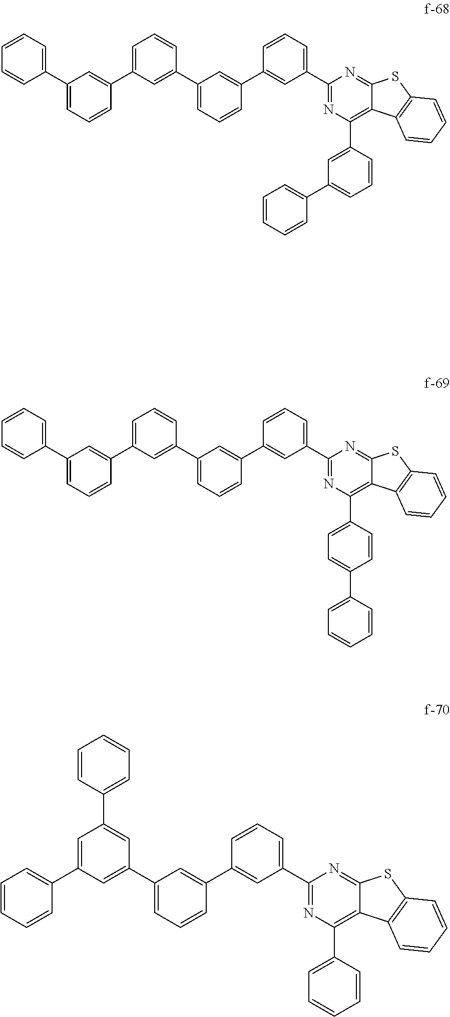 [SMILES: c1ccc(-c2cccc(-c3cccc(-c4cccc(-c5cccc(-c6nc(-c7cccc(-c8ccccc8)c7)c7c(n6)sc6ccccc67)c5)c4)c3)c2)cc1 ; c1ccc(-c2ccc(-c3nc(-c4cccc(-c5cccc(-c6cccc(-c7cccc(-c8ccccc8)c7)c6)c5)c4)nc4sc5ccccc5c34)cc2)cc1 ; c1ccc(-c2cc(-c3ccccc3)cc(-c3cccc(-c4cccc(-c5nc(-c6ccccc6)c6c(n5)sc5ccccc56)c4)c3)c2)cc1]

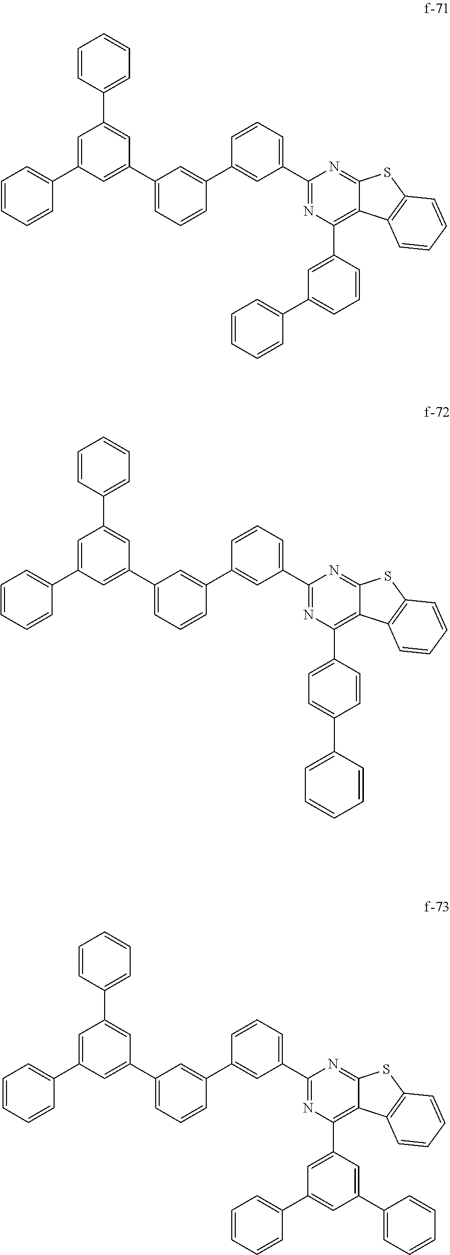 [SMILES: c1ccc(-c2cc(-c3ccccc3)cc(-c3cccc(-c4cccc(-c5nc(-c6cccc(-c7ccccc7)c6)c6c(n5)sc5ccccc56)c4)c3)c2)cc1 ; c1ccc(-c2ccc(-c3nc(-c4cccc(-c5cccc(-c6cc(-c7ccccc7)cc(-c7ccccc7)c6)c5)c4)nc4sc5ccccc5c34)cc2)cc1 ; c1ccc(-c2cc(-c3ccccc3)cc(-c3cccc(-c4cccc(-c5nc(-c6cc(-c7ccccc7)cc(-c7ccccc7)c6)c6c(n5)sc5ccccc56)c4)c3)c2)cc1]

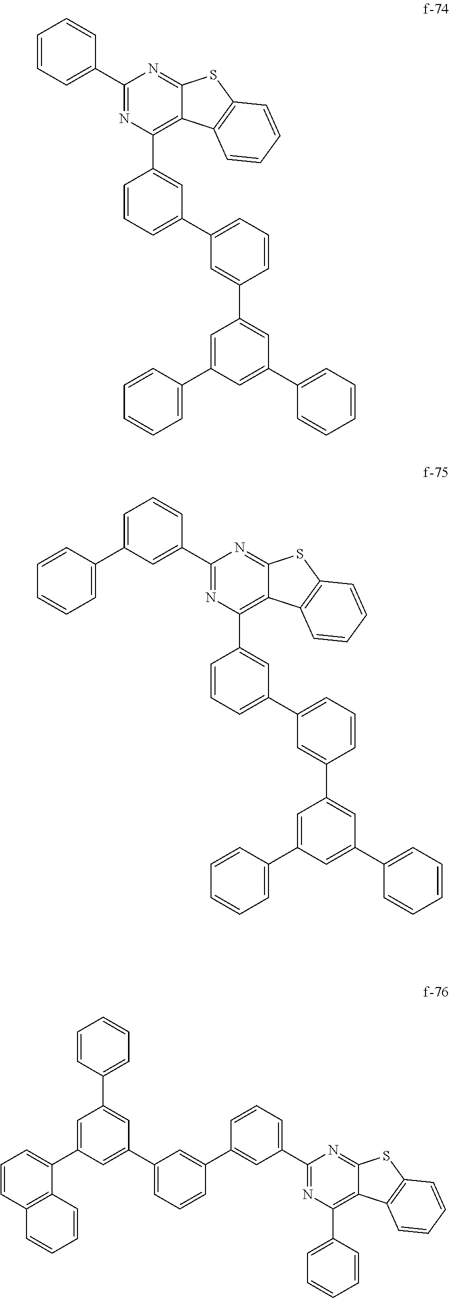 [SMILES: c1ccc(-c2cc(-c3ccccc3)cc(-c3cccc(-c4cccc(-c5nc(-c6ccccc6)nc6sc7ccccc7c56)c4)c3)c2)cc1 ; c1ccc(-c2cc(-c3ccccc3)cc(-c3cccc(-c4cccc(-c5nc(-c6cccc(-c7ccccc7)c6)nc6sc7ccccc7c56)c4)c3)c2)cc1 ; c1ccc(-c2cc(-c3cccc(-c4cccc(-c5nc(-c6ccccc6)c6c(n5)sc5ccccc56)c4)c3)cc(-c3cccc4ccccc34)c2)cc1]

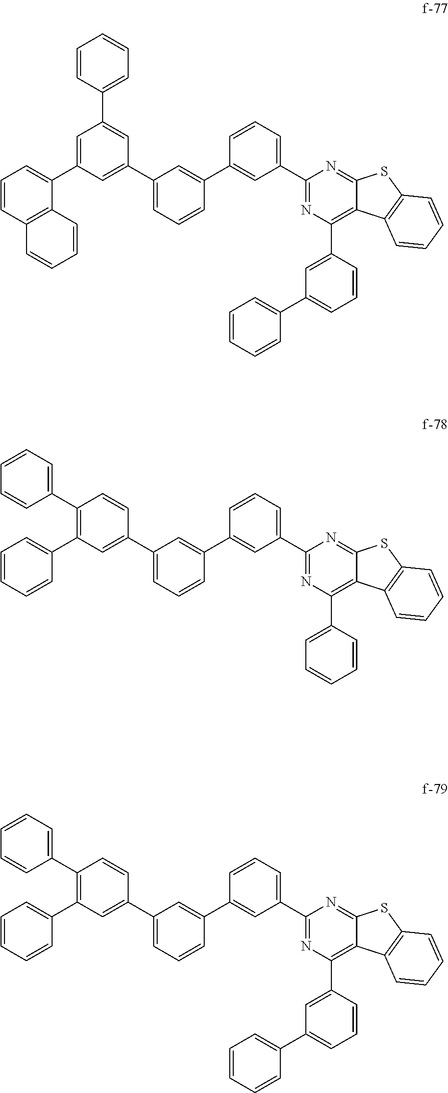 [SMILES: c1ccc(-c2cc(-c3cccc(-c4cccc(-c5nc(-c6cccc(-c7ccccc7)c6)c6c(n5)sc5ccccc56)c4)c3)cc(-c3cccc4ccccc34)c2)cc1 ; c1ccc(-c2ccc(-c3cccc(-c4cccc(-c5nc(-c6ccccc6)c6c(n5)sc5ccccc56)c4)c3)cc2-c2ccccc2)cc1 ; c1ccc(-c2cccc(-c3nc(-c4cccc(-c5cccc(-c6ccc(-c7ccccc7)c(-c7ccccc7)c6)c5)c4)nc4sc5ccccc5c34)c2)cc1]

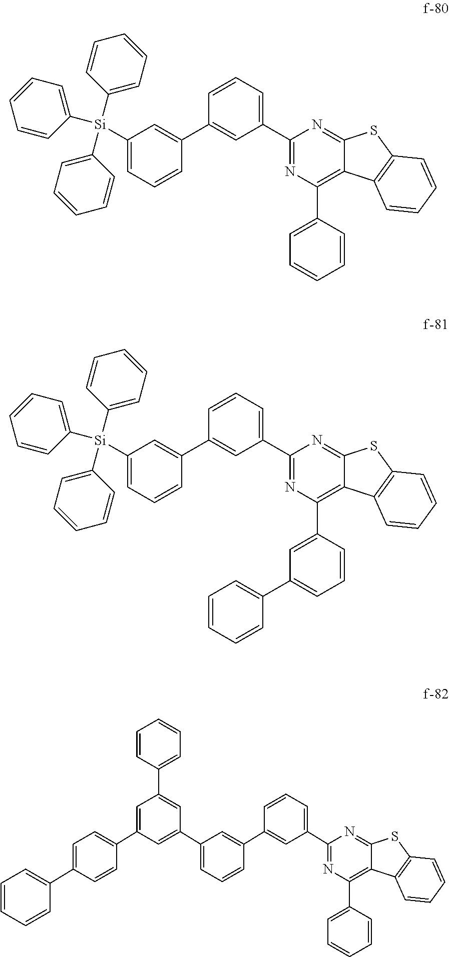 [SMILES: c1ccc(-c2nc(-c3cccc(-c4cccc([Si](c5ccccc5)(c5ccccc5)c5ccccc5)c4)c3)nc3sc4ccccc4c23)cc1 ; c1ccc(-c2cccc(-c3nc(-c4cccc(-c5cccc([Si](c6ccccc6)(c6ccccc6)c6ccccc6)c5)c4)nc4sc5ccccc5c34)c2)cc1 ; c1ccc(-c2ccc(-c3cc(-c4ccccc4)cc(-c4cccc(-c5cccc(-c6nc(-c7ccccc7)c7c(n6)sc6ccccc67)c5)c4)c3)cc2)cc1]

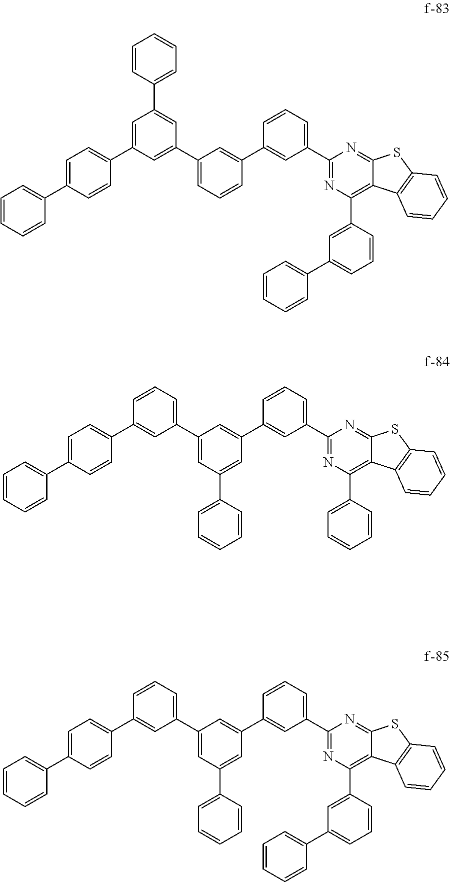 [SMILES: c1ccc(-c2ccc(-c3cc(-c4ccccc4)cc(-c4cccc(-c5cccc(-c6nc(-c7cccc(-c8ccccc8)c7)c7c(n6)sc6ccccc67)c5)c4)c3)cc2)cc1 ; c1ccc(-c2ccc(-c3cccc(-c4cc(-c5ccccc5)cc(-c5cccc(-c6nc(-c7ccccc7)c7c(n6)sc6ccccc67)c5)c4)c3)cc2)cc1 ; c1ccc(-c2ccc(-c3cccc(-c4cc(-c5ccccc5)cc(-c5cccc(-c6nc(-c7cccc(-c8ccccc8)c7)c7c(n6)sc6ccccc67)c5)c4)c3)cc2)cc1]

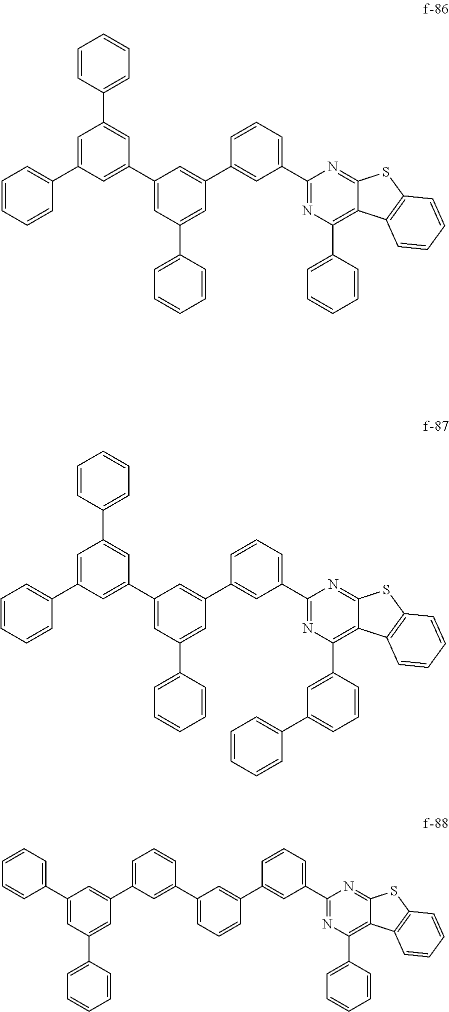 [SMILES: c1ccc(-c2cc(-c3ccccc3)cc(-c3cc(-c4ccccc4)cc(-c4cccc(-c5nc(-c6ccccc6)c6c(n5)sc5ccccc56)c4)c3)c2)cc1 ; c1ccc(-c2cc(-c3ccccc3)cc(-c3cc(-c4ccccc4)cc(-c4cccc(-c5nc(-c6cccc(-c7ccccc7)c6)c6c(n5)sc5ccccc56)c4)c3)c2)cc1 ; c1ccc(-c2cc(-c3ccccc3)cc(-c3cccc(-c4cccc(-c5cccc(-c6nc(-c7ccccc7)c7c(n6)sc6ccccc67)c5)c4)c3)c2)cc1]

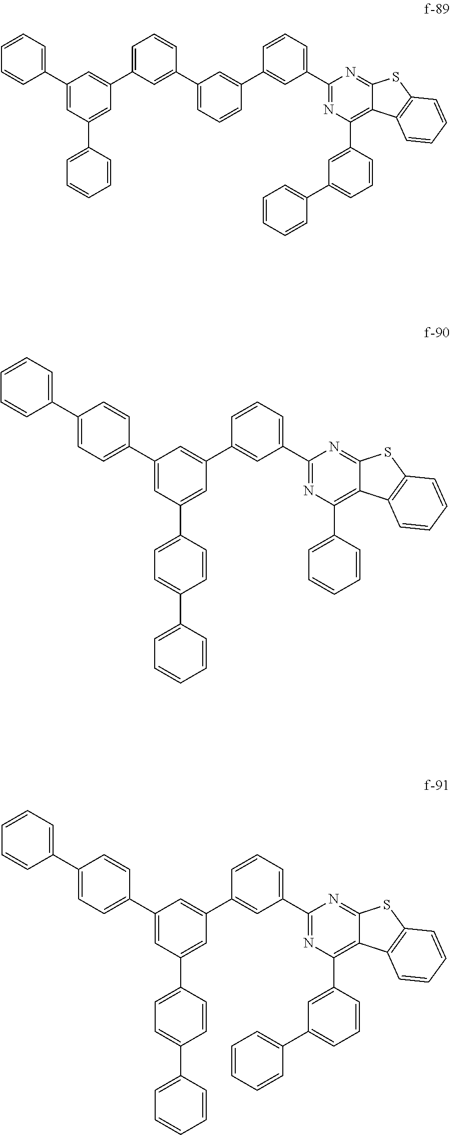 [SMILES: c1ccc(-c2cc(-c3ccccc3)cc(-c3cccc(-c4cccc(-c5cccc(-c6nc(-c7cccc(-c8ccccc8)c7)c7c(n6)sc6ccccc67)c5)c4)c3)c2)cc1 ; c1ccc(-c2ccc(-c3cc(-c4ccc(-c5ccccc5)cc4)cc(-c4cccc(-c5nc(-c6ccccc6)c6c(n5)sc5ccccc56)c4)c3)cc2)cc1 ; c1ccc(-c2ccc(-c3cc(-c4ccc(-c5ccccc5)cc4)cc(-c4cccc(-c5nc(-c6cccc(-c7ccccc7)c6)c6c(n5)sc5ccccc56)c4)c3)cc2)cc1]

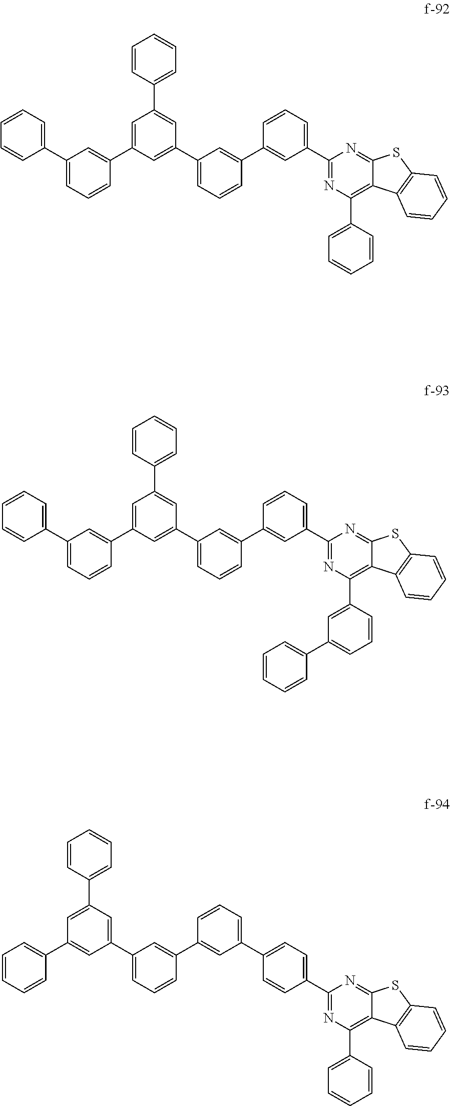 [SMILES: c1ccc(-c2cccc(-c3cc(-c4ccccc4)cc(-c4cccc(-c5cccc(-c6nc(-c7ccccc7)c7c(n6)sc6ccccc67)c5)c4)c3)c2)cc1 ; c1ccc(-c2cccc(-c3cc(-c4ccccc4)cc(-c4cccc(-c5cccc(-c6nc(-c7cccc(-c8ccccc8)c7)c7c(n6)sc6ccccc67)c5)c4)c3)c2)cc1 ; c1ccc(-c2cc(-c3ccccc3)cc(-c3cccc(-c4cccc(-c5ccc(-c6nc(-c7ccccc7)c7c(n6)sc6ccccc67)cc5)c4)c3)c2)cc1]

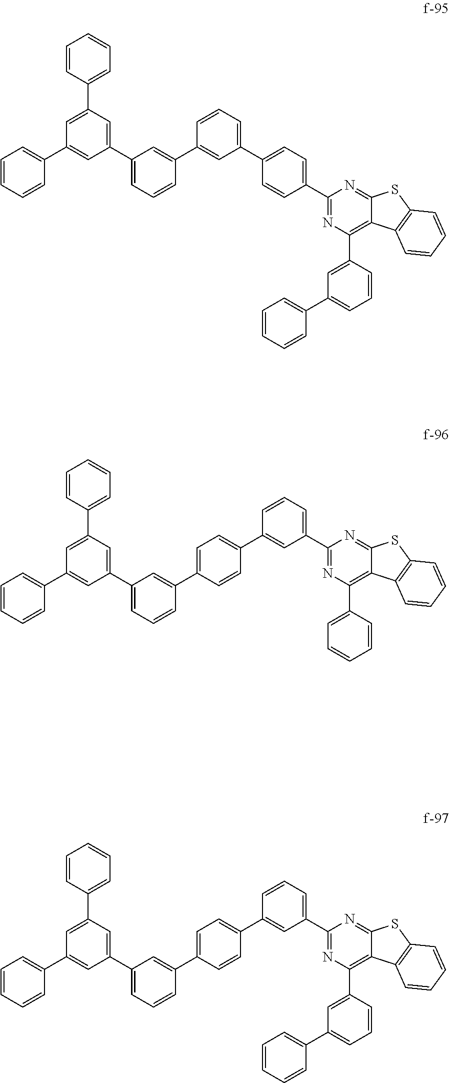 [SMILES: c1ccc(-c2cc(-c3ccccc3)cc(-c3cccc(-c4cccc(-c5ccc(-c6nc(-c7cccc(-c8ccccc8)c7)c7c(n6)sc6ccccc67)cc5)c4)c3)c2)cc1 ; c1ccc(-c2cc(-c3ccccc3)cc(-c3cccc(-c4ccc(-c5cccc(-c6nc(-c7ccccc7)c7c(n6)sc6ccccc67)c5)cc4)c3)c2)cc1 ; c1ccc(-c2cc(-c3ccccc3)cc(-c3cccc(-c4ccc(-c5cccc(-c6nc(-c7cccc(-c8ccccc8)c7)c7c(n6)sc6ccccc67)c5)cc4)c3)c2)cc1]

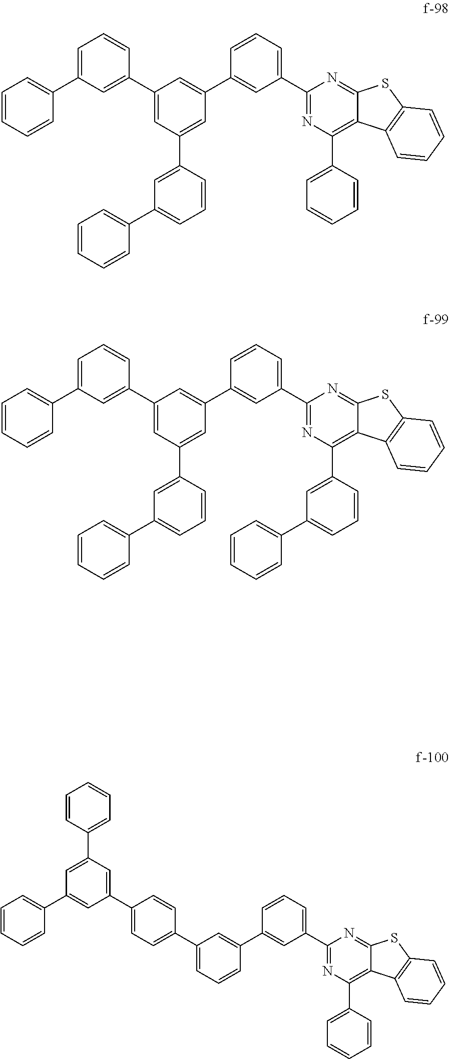 [SMILES: c1ccc(-c2cccc(-c3cc(-c4cccc(-c5ccccc5)c4)cc(-c4cccc(-c5nc(-c6ccccc6)c6c(n5)sc5ccccc56)c4)c3)c2)cc1 ; c1ccc(-c2cccc(-c3cc(-c4cccc(-c5ccccc5)c4)cc(-c4cccc(-c5nc(-c6cccc(-c7ccccc7)c6)c6c(n5)sc5ccccc56)c4)c3)c2)cc1 ; c1ccc(-c2cc(-c3ccccc3)cc(-c3ccc(-c4cccc(-c5cccc(-c6nc(-c7ccccc7)c7c(n6)sc6ccccc67)c5)c4)cc3)c2)cc1]

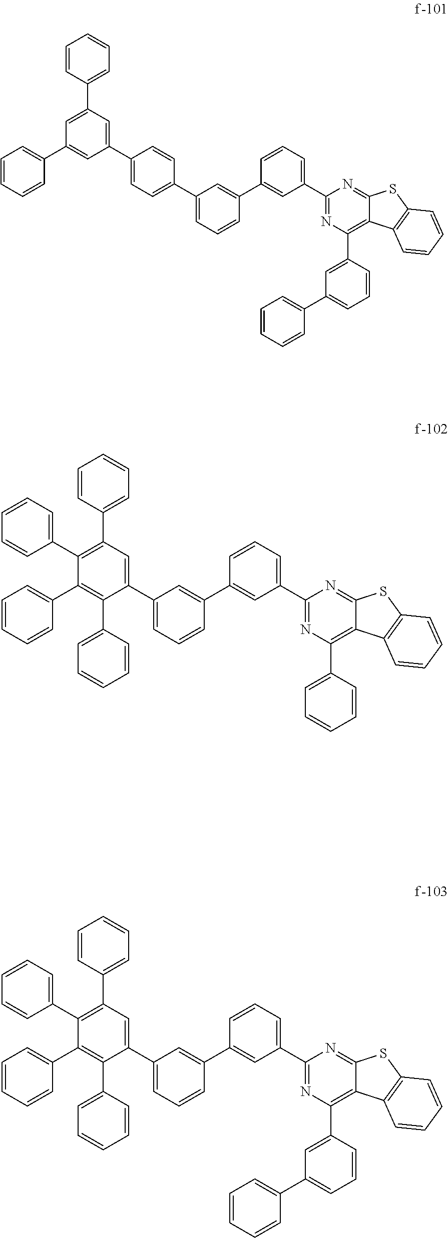 [SMILES: c1ccc(-c2cc(-c3ccccc3)cc(-c3ccc(-c4cccc(-c5cccc(-c6nc(-c7cccc(-c8ccccc8)c7)c7c(n6)sc6ccccc67)c5)c4)cc3)c2)cc1 ; c1ccc(-c2cc(-c3cccc(-c4cccc(-c5nc(-c6ccccc6)c6c(n5)sc5ccccc56)c4)c3)c(-c3ccccc3)c(-c3ccccc3)c2-c2ccccc2)cc1 ; c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4sc5ccccc5c34)c2)cc1]

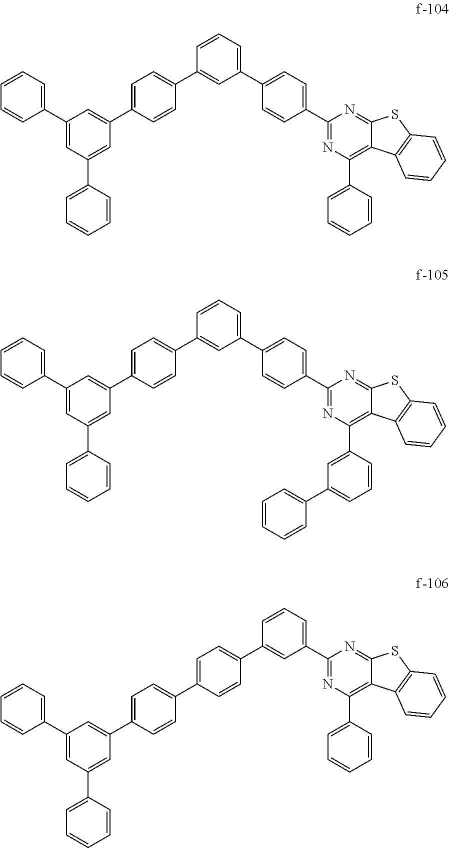 [SMILES: c1ccc(-c2cc(-c3ccccc3)cc(-c3ccc(-c4cccc(-c5ccc(-c6nc(-c7ccccc7)c7c(n6)sc6ccccc67)cc5)c4)cc3)c2)cc1 ; c1ccc(-c2cc(-c3ccccc3)cc(-c3ccc(-c4cccc(-c5ccc(-c6nc(-c7cccc(-c8ccccc8)c7)c7c(n6)sc6ccccc67)cc5)c4)cc3)c2)cc1 ; c1ccc(-c2cc(-c3ccccc3)cc(-c3ccc(-c4ccc(-c5cccc(-c6nc(-c7ccccc7)c7c(n6)sc6ccccc67)c5)cc4)cc3)c2)cc1]

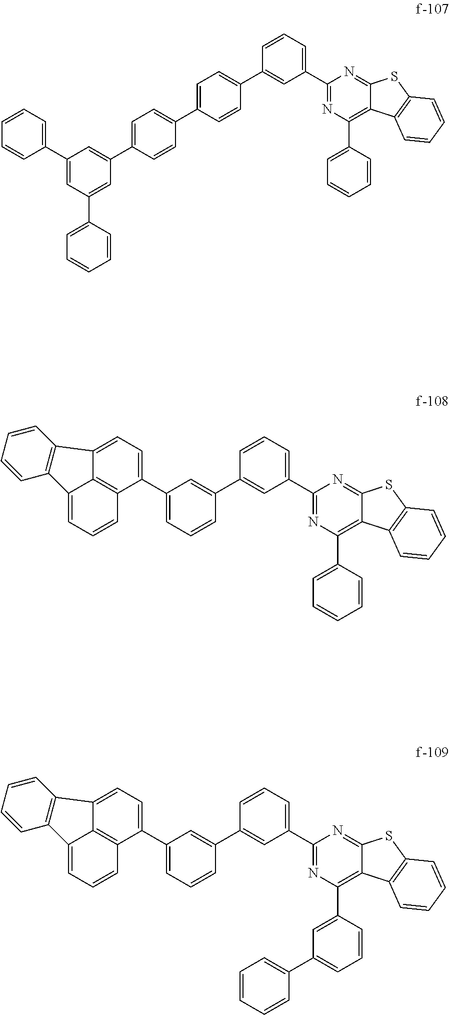 [SMILES: c1ccc(-c2cc(-c3ccccc3)cc(-c3ccc(-c4ccc(-c5cccc(-c6nc(-c7ccccc7)c7c(n6)sc6ccccc67)c5)cc4)cc3)c2)cc1 ; c1ccc(-c2nc(-c3cccc(-c4cccc(-c5ccc6c7c(cccc57)-c5ccccc5-6)c4)c3)nc3sc4ccccc4c23)cc1 ; c1ccc(-c2cccc(-c3nc(-c4cccc(-c5cccc(-c6ccc7c8c(cccc68)-c6ccccc6-7)c5)c4)nc4sc5ccccc5c34)c2)cc1]

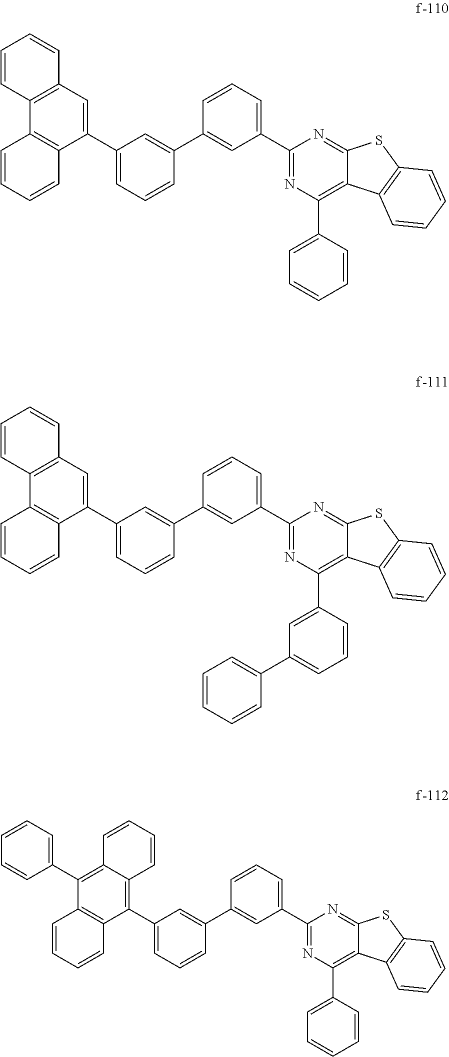 [SMILES: c1ccc(-c2nc(-c3cccc(-c4cccc(-c5cc6ccccc6c6ccccc56)c4)c3)nc3sc4ccccc4c23)cc1 ; c1ccc(-c2cccc(-c3nc(-c4cccc(-c5cccc(-c6cc7ccccc7c7ccccc67)c5)c4)nc4sc5ccccc5c34)c2)cc1 ; c1ccc(-c2c3ccccc3c(-c3cccc(-c4cccc(-c5nc(-c6ccccc6)c6c(n5)sc5ccccc56)c4)c3)c3ccccc23)cc1]

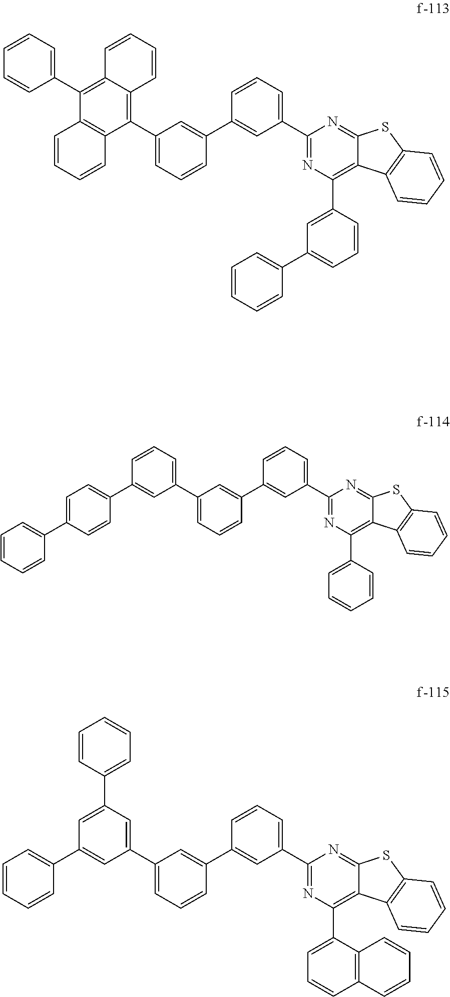 [SMILES: c1ccc(-c2cccc(-c3nc(-c4cccc(-c5cccc(-c6c7ccccc7c(-c7ccccc7)c7ccccc67)c5)c4)nc4sc5ccccc5c34)c2)cc1 ; c1ccc(-c2ccc(-c3cccc(-c4cccc(-c5cccc(-c6nc(-c7ccccc7)c7c(n6)sc6ccccc67)c5)c4)c3)cc2)cc1 ; c1ccc(-c2cc(-c3ccccc3)cc(-c3cccc(-c4cccc(-c5nc(-c6cccc7ccccc67)c6c(n5)sc5ccccc56)c4)c3)c2)cc1]

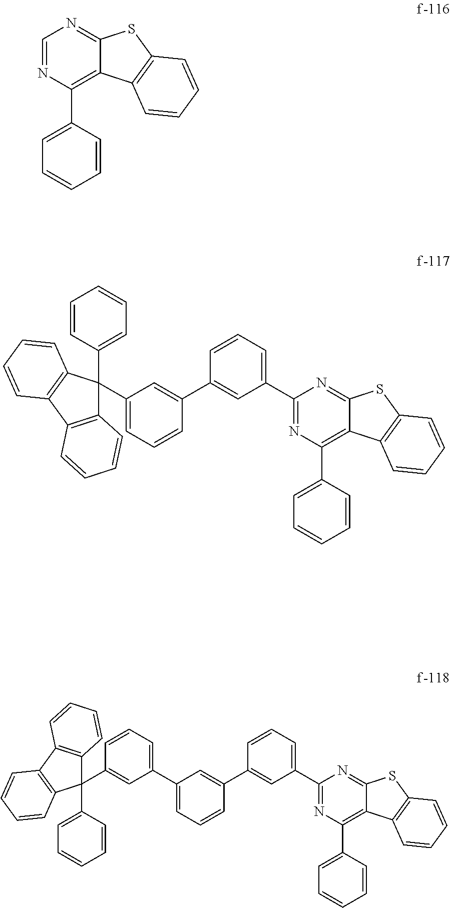 [SMILES: c1ccc(-c2ncnc3sc4ccccc4c23)cc1 ; c1ccc(-c2nc(-c3cccc(-c4cccc(C5(c6ccccc6)c6ccccc6-c6ccccc65)c4)c3)nc3sc4ccccc4c23)cc1 ; c1ccc(-c2nc(-c3cccc(-c4cccc(-c5cccc(C6(c7ccccc7)c7ccccc7-c7ccccc76)c5)c4)c3)nc3sc4ccccc4c23)cc1]

Group of X₁=Si(R₄)(R₅) in Formula 1-2

(R₄ and R₅ are described in the present specification)

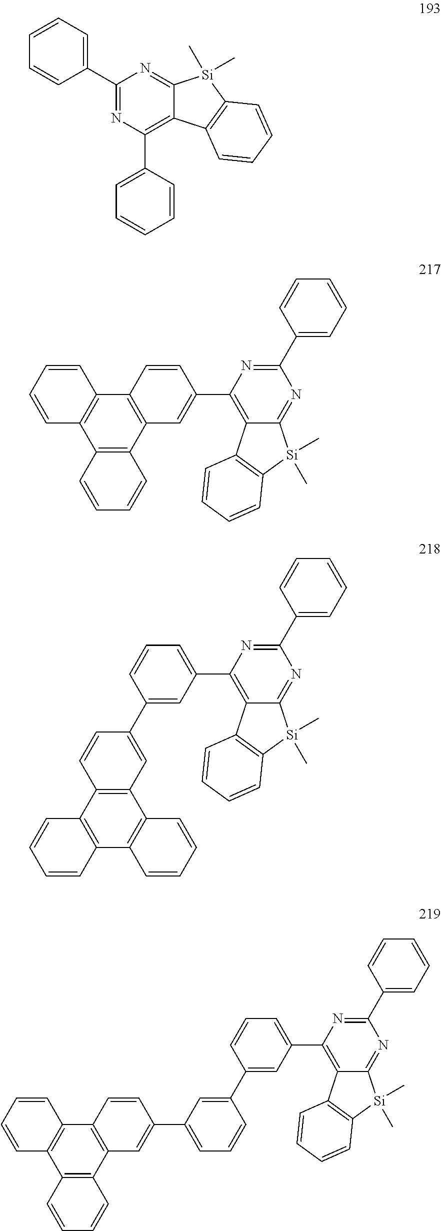 [SMILES: C[Si]1(C)c2ccccc2-c2c(-c3ccccc3)nc(-c3ccccc3)nc21 ; C[Si]1(C)c2ccccc2-c2c(-c3ccc4c5ccccc5c5ccccc5c4c3)nc(-c3ccccc3)nc21 ; C[Si]1(C)c2ccccc2-c2c(-c3cccc(-c4ccc5c6ccccc6c6ccccc6c5c4)c3)nc(-c3ccccc3)nc21 ; C[Si]1(C)c2ccccc2-c2c(-c3cccc(-c4cccc(-c5ccc6c7ccccc7c7ccccc7c6c5)c4)c3)nc(-c3ccccc3)nc21]

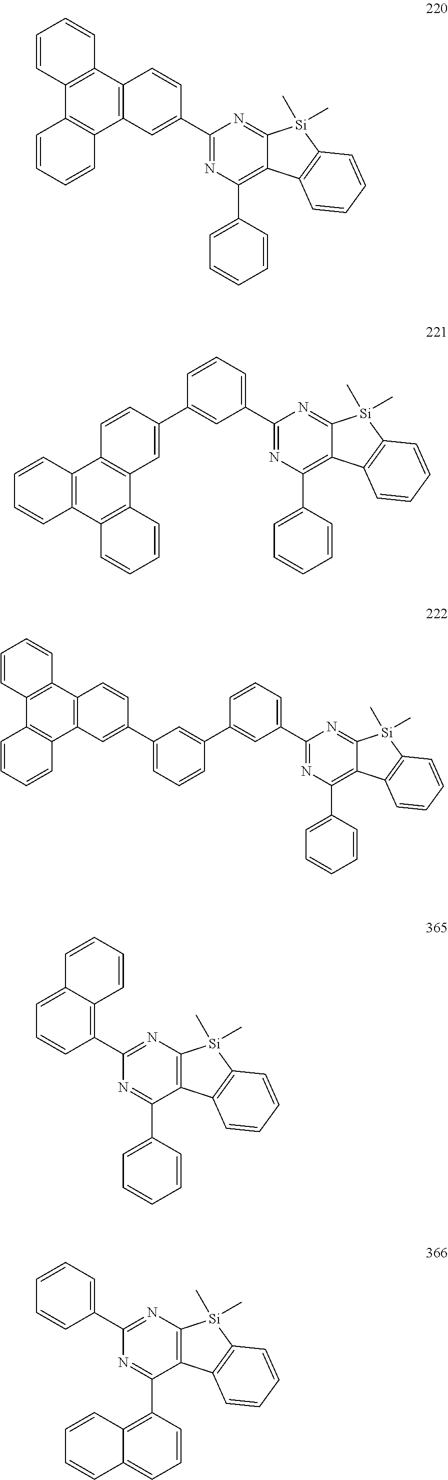 [SMILES: C[Si]1(C)c2ccccc2-c2c(-c3ccccc3)nc(-c3ccc4c5ccccc5c5ccccc5c4c3)nc21 ; C[Si]1(C)c2ccccc2-c2c(-c3ccccc3)nc(-c3cccc(-c4ccc5c6ccccc6c6ccccc6c5c4)c3)nc21 ; C[Si]1(C)c2ccccc2-c2c(-c3ccccc3)nc(-c3cccc(-c4cccc(-c5ccc6c7ccccc7c7ccccc7c6c5)c4)c3)nc21 ; C[Si]1(C)c2ccccc2-c2c(-c3ccccc3)nc(-c3cccc4ccccc34)nc21 ; C[Si]1(C)c2ccccc2-c2c(-c3cccc4ccccc34)nc(-c3ccccc3)nc21]

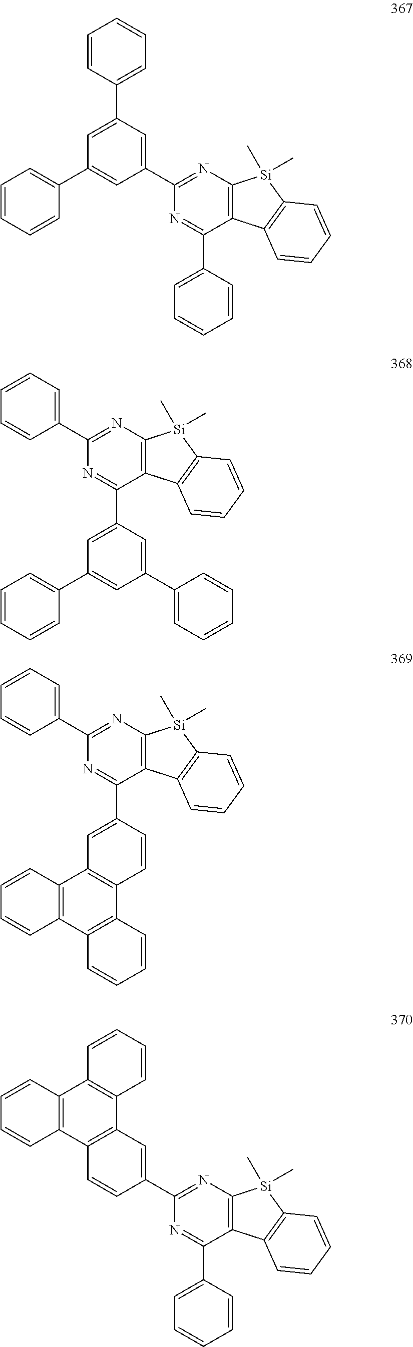

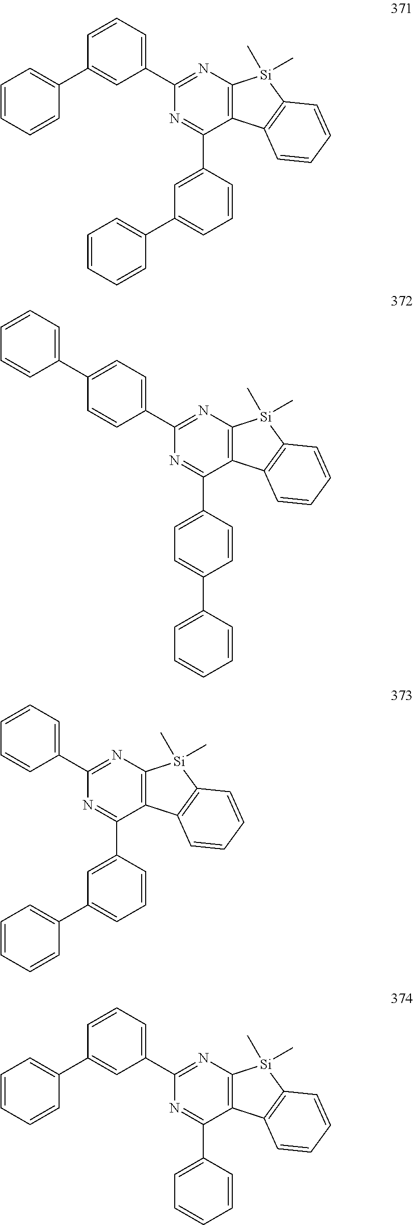 [SMILES: C[Si]1(C)c2ccccc2-c2c(-c3cccc(-c4ccccc4)c3)nc(-c3cccc(-c4ccccc4)c3)nc21 ; C[Si]1(C)c2ccccc2-c2c(-c3ccc(-c4ccccc4)cc3)nc(-c3ccc(-c4ccccc4)cc3)nc21 ; C[Si]1(C)c2ccccc2-c2c(-c3cccc(-c4ccccc4)c3)nc(-c3ccccc3)nc21 ; C[Si]1(C)c2ccccc2-c2c(-c3ccccc3)nc(-c3cccc(-c4ccccc4)c3)nc21]

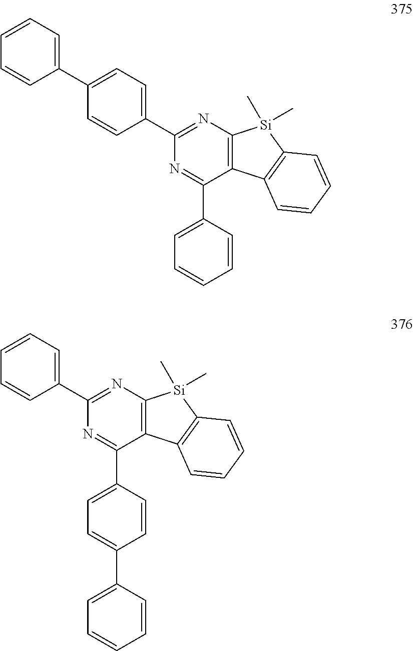 [SMILES: C[Si]1(C)c2ccccc2-c2c(-c3ccccc3)nc(-c3ccc(-c4ccccc4)cc3)nc21 ; C[Si]1(C)c2ccccc2-c2c(-c3ccc(-c4ccccc4)cc3)nc(-c3ccccc3)nc21]

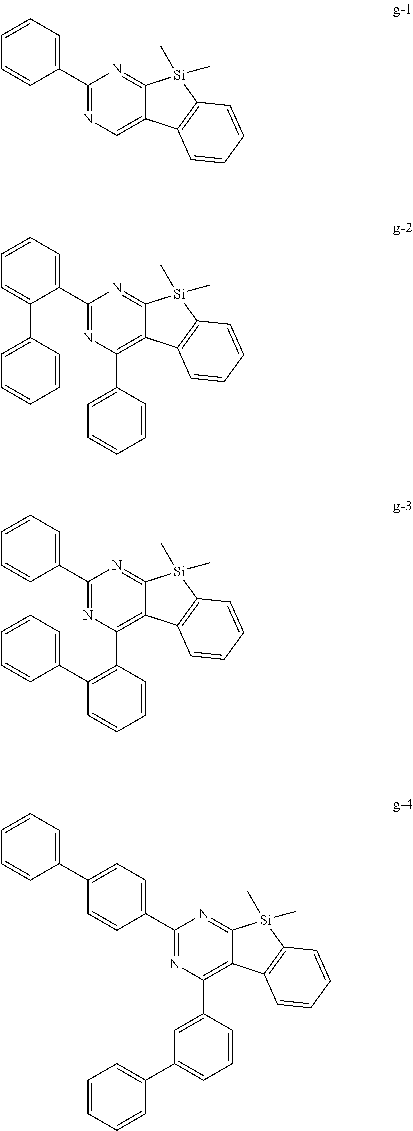 [SMILES: C[Si]1(C)c2ccccc2-c2cnc(-c3ccccc3)nc21 ; C[Si]1(C)c2ccccc2-c2c(-c3ccccc3)nc(-c3ccccc3-c3ccccc3)nc21 ; C[Si]1(C)c2ccccc2-c2c(-c3ccccc3-c3ccccc3)nc(-c3ccccc3)nc21 ; C[Si]1(C)c2ccccc2-c2c(-c3cccc(-c4ccccc4)c3)nc(-c3ccc(-c4ccccc4)cc3)nc21]

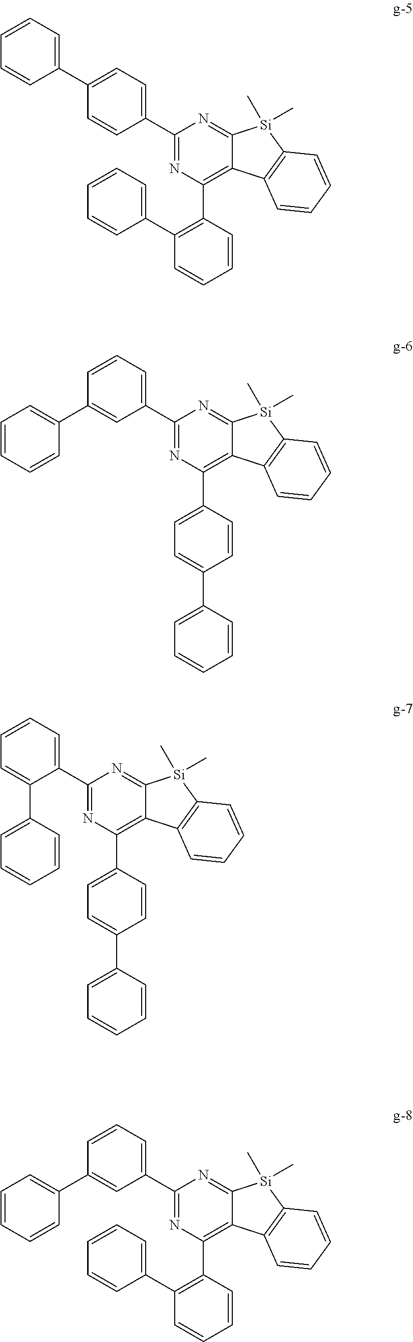 [SMILES: C[Si]1(C)c2ccccc2-c2c(-c3ccccc3-c3ccccc3)nc(-c3ccc(-c4ccccc4)cc3)nc21 ; C[Si]1(C)c2ccccc2-c2c(-c3ccc(-c4ccccc4)cc3)nc(-c3cccc(-c4ccccc4)c3)nc21 ; C[Si]1(C)c2ccccc2-c2c(-c3ccc(-c4ccccc4)cc3)nc(-c3ccccc3-c3ccccc3)nc21 ; C[Si]1(C)c2ccccc2-c2c(-c3ccccc3-c3ccccc3)nc(-c3cccc(-c4ccccc4)c3)nc21]

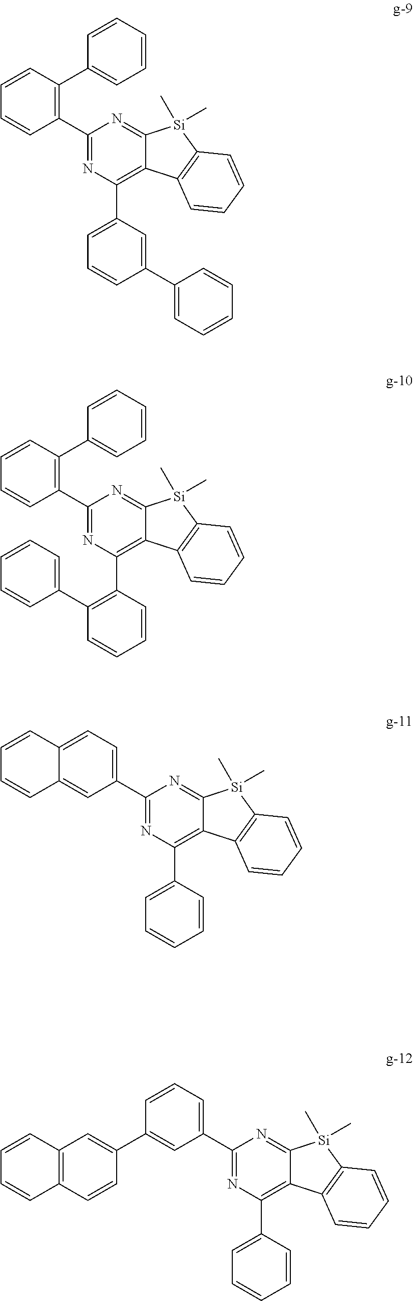 [SMILES: C[Si]1(C)c2ccccc2-c2c(-c3cccc(-c4ccccc4)c3)nc(-c3ccccc3-c3ccccc3)nc21 ; C[Si]1(C)c2ccccc2-c2c(-c3ccccc3-c3ccccc3)nc(-c3ccccc3-c3ccccc3)nc21 ; C[Si]1(C)c2ccccc2-c2c(-c3ccccc3)nc(-c3ccc4ccccc4c3)nc21 ; C[Si]1(C)c2ccccc2-c2c(-c3ccccc3)nc(-c3cccc(-c4ccc5ccccc5c4)c3)nc21]

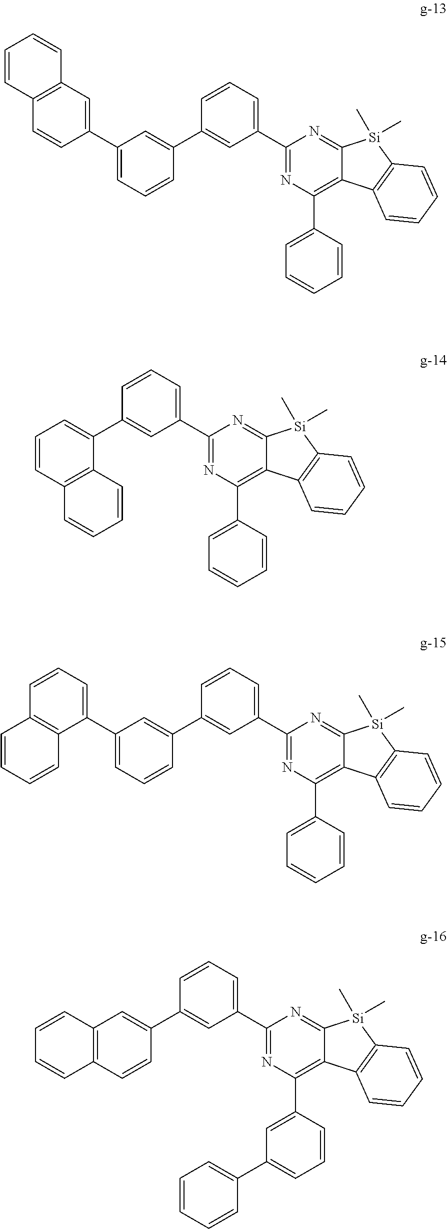 [SMILES: C[Si]1(C)c2ccccc2-c2c(-c3ccccc3)nc(-c3cccc(-c4cccc(-c5ccc6ccccc6c5)c4)c3)nc21 ; C[Si]1(C)c2ccccc2-c2c(-c3ccccc3)nc(-c3cccc(-c4cccc5ccccc45)c3)nc21 ; C[Si]1(C)c2ccccc2-c2c(-c3ccccc3)nc(-c3cccc(-c4cccc(-c5cccc6ccccc56)c4)c3)nc21 ; C[Si]1(C)c2ccccc2-c2c(-c3cccc(-c4ccccc4)c3)nc(-c3cccc(-c4ccc5ccccc5c4)c3)nc21]

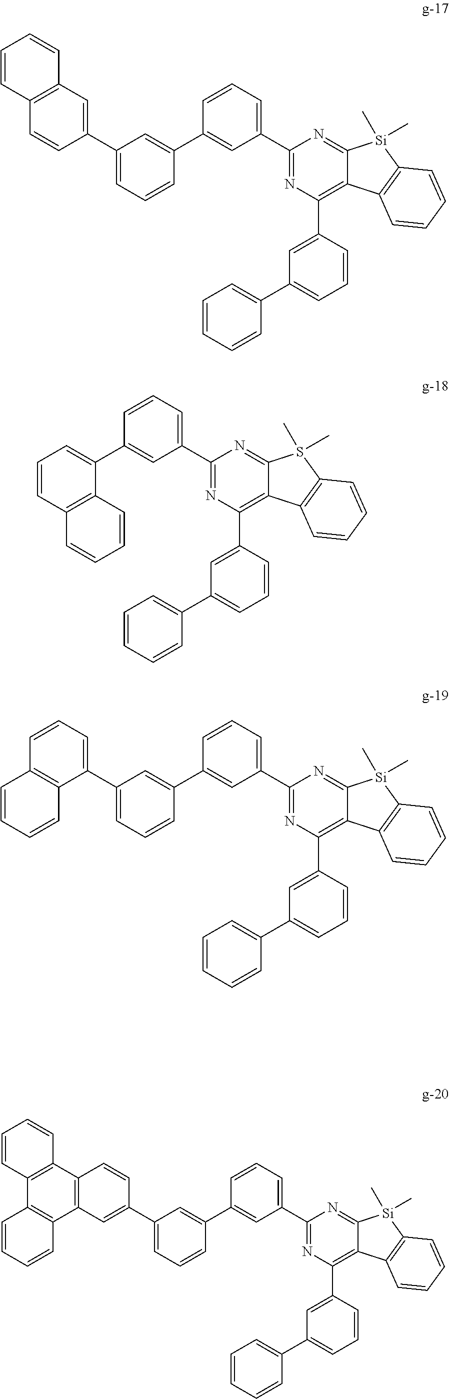 [SMILES: C[Si]1(C)c2ccccc2-c2c(-c3cccc(-c4ccccc4)c3)nc(-c3cccc(-c4cccc(-c5ccc6ccccc6c5)c4)c3)nc21 ; CS1(C)c2ccccc2-c2c(-c3cccc(-c4ccccc4)c3)nc(-c3cccc(-c4cccc5ccccc45)c3)nc21 ; C[Si]1(C)c2ccccc2-c2c(-c3cccc(-c4ccccc4)c3)nc(-c3cccc(-c4cccc(-c5cccc6ccccc56)c4)c3)nc21 ; C[Si]1(C)c2ccccc2-c2c(-c3cccc(-c4ccccc4)c3)nc(-c3cccc(-c4cccc(-c5ccc6c7ccccc7c7ccccc7c6c5)c4)c3)nc21]

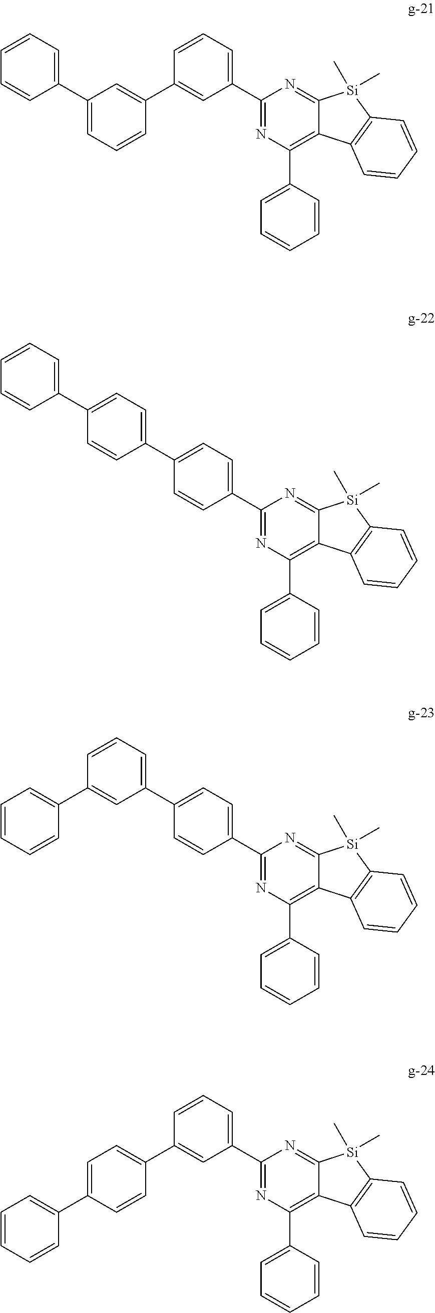 [SMILES: C[Si]1(C)c2ccccc2-c2c(-c3ccccc3)nc(-c3cccc(-c4cccc(-c5ccccc5)c4)c3)nc21 ; C[Si]1(C)c2ccccc2-c2c(-c3ccccc3)nc(-c3ccc(-c4ccc(-c5ccccc5)cc4)cc3)nc21 ; C[Si]1(C)c2ccccc2-c2c(-c3ccccc3)nc(-c3ccc(-c4cccc(-c5ccccc5)c4)cc3)nc21 ; C[Si]1(C)c2ccccc2-c2c(-c3ccccc3)nc(-c3cccc(-c4ccc(-c5ccccc5)cc4)c3)nc21]

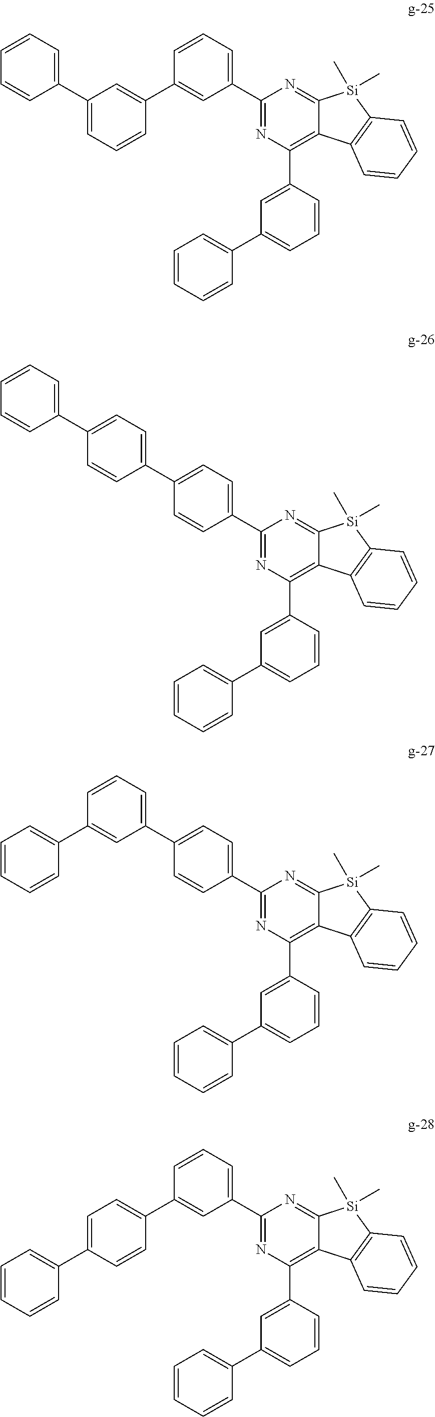 [SMILES: C[Si]1(C)c2ccccc2-c2c(-c3cccc(-c4ccccc4)c3)nc(-c3cccc(-c4cccc(-c5ccccc5)c4)c3)nc21 ; C[Si]1(C)c2ccccc2-c2c(-c3cccc(-c4ccccc4)c3)nc(-c3ccc(-c4ccc(-c5ccccc5)cc4)cc3)nc21 ; C[Si]1(C)c2ccccc2-c2c(-c3cccc(-c4ccccc4)c3)nc(-c3ccc(-c4cccc(-c5ccccc5)c4)cc3)nc21 ; C[Si]1(C)c2ccccc2-c2c(-c3cccc(-c4ccccc4)c3)nc(-c3cccc(-c4ccc(-c5ccccc5)cc4)c3)nc21]

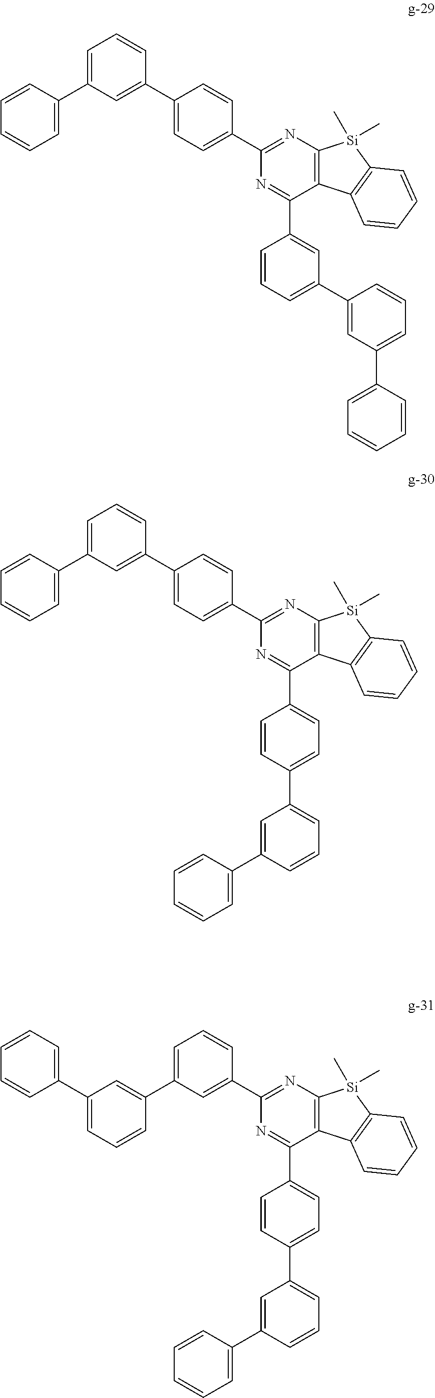 [SMILES: C[Si]1(C)c2ccccc2-c2c(-c3cccc(-c4cccc(-c5ccccc5)c4)c3)nc(-c3ccc(-c4cccc(-c5ccccc5)c4)cc3)nc21 ; C[Si]1(C)c2ccccc2-c2c(-c3ccc(-c4cccc(-c5ccccc5)c4)cc3)nc(-c3ccc(-c4cccc(-c5ccccc5)c4)cc3)nc21 ; C[Si]1(C)c2ccccc2-c2c(-c3ccc(-c4cccc(-c5ccccc5)c4)cc3)nc(-c3cccc(-c4cccc(-c5ccccc5)c4)c3)nc21]

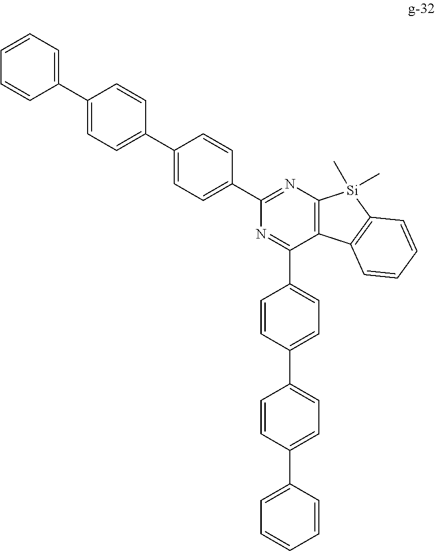 [SMILES: C[Si]1(C)c2ccccc2-c2c(-c3ccc(-c4ccc(-c5ccccc5)cc4)cc3)nc(-c3ccc(-c4ccc(-c5ccccc5)cc4)cc3)nc21]

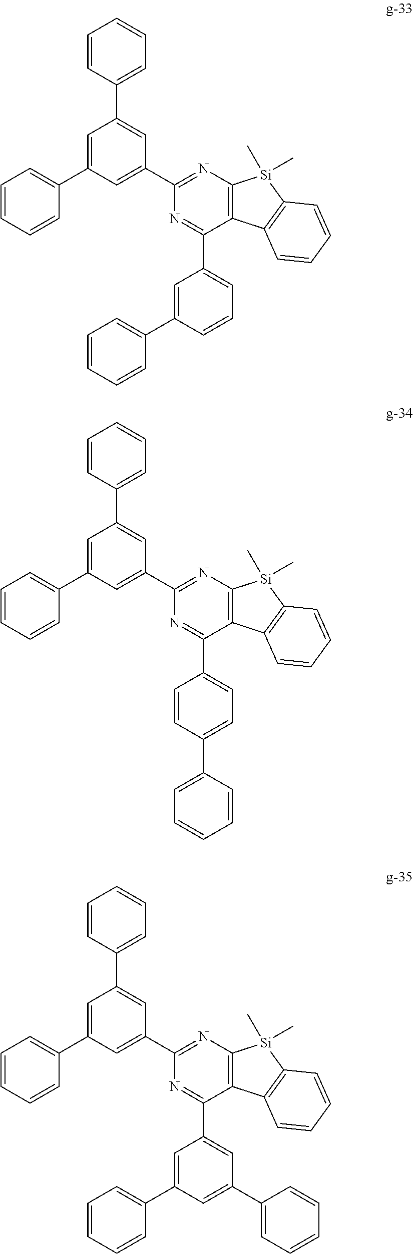

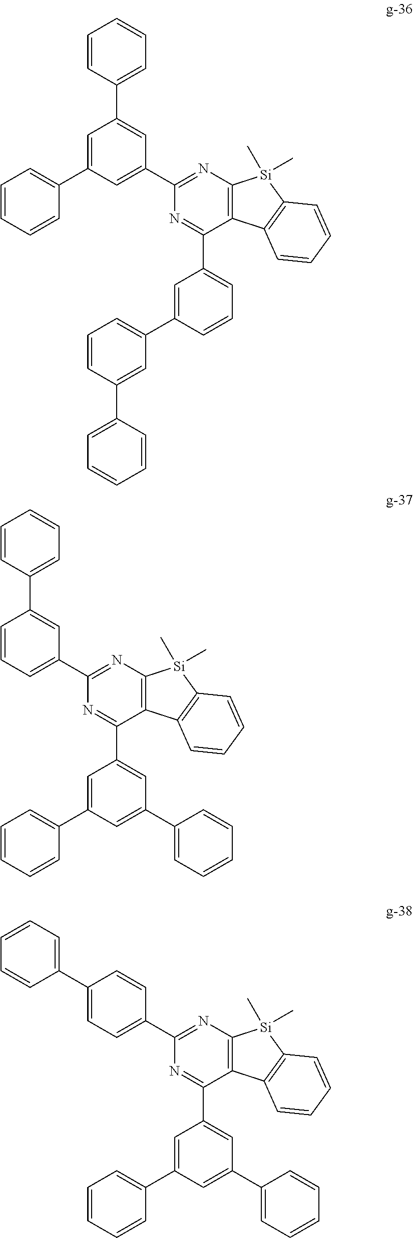

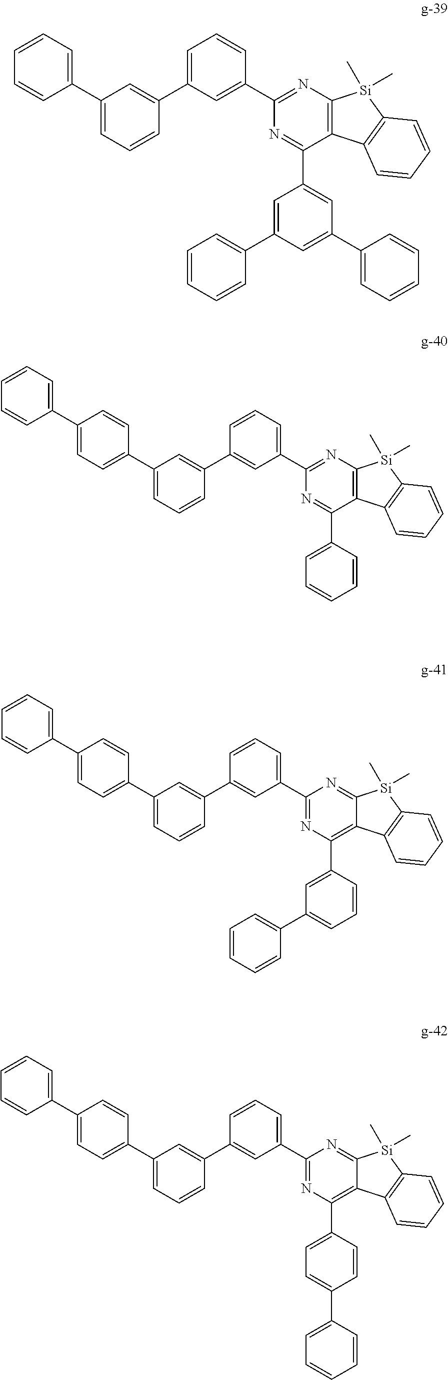 [SMILES: C[Si]1(C)c2ccccc2-c2c(-c3cc(-c4ccccc4)cc(-c4ccccc4)c3)nc(-c3cccc(-c4cccc(-c5ccccc5)c4)c3)nc21 ; C[Si]1(C)c2ccccc2-c2c(-c3ccccc3)nc(-c3cccc(-c4cccc(-c5ccc(-c6ccccc6)cc5)c4)c3)nc21 ; C[Si]1(C)c2ccccc2-c2c(-c3cccc(-c4ccccc4)c3)nc(-c3cccc(-c4cccc(-c5ccc(-c6ccccc6)cc5)c4)c3)nc21 ; C[Si]1(C)c2ccccc2-c2c(-c3ccc(-c4ccccc4)cc3)nc(-c3cccc(-c4cccc(-c5ccc(-c6ccccc6)cc5)c4)c3)nc21]

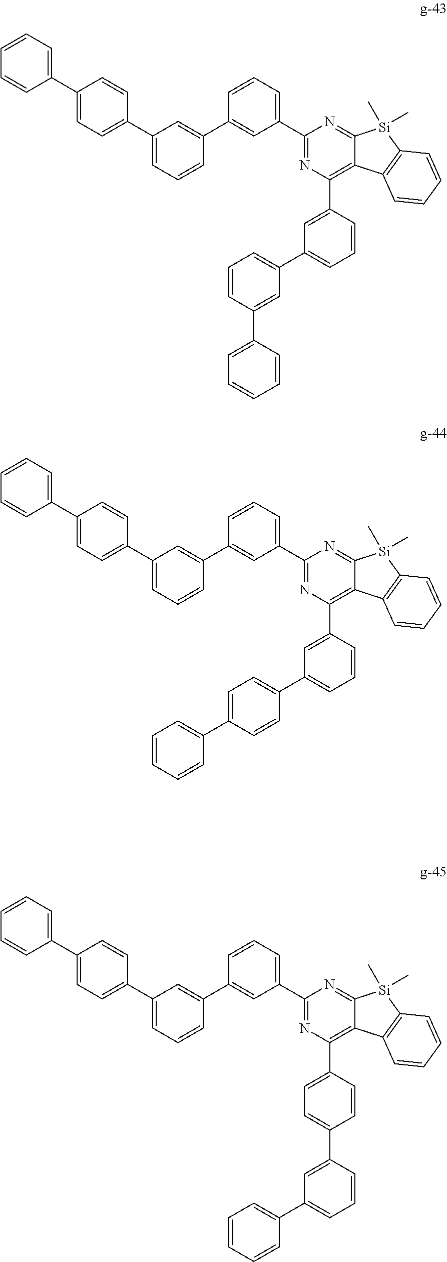 [SMILES: C[Si]1(C)c2ccccc2-c2c(-c3cccc(-c4cccc(-c5ccccc5)c4)c3)nc(-c3cccc(-c4cccc(-c5ccc(-c6ccccc6)cc5)c4)c3)nc21 ; C[Si]1(C)c2ccccc2-c2c(-c3cccc(-c4ccc(-c5ccccc5)cc4)c3)nc(-c3cccc(-c4cccc(-c5ccc(-c6ccccc6)cc5)c4)c3)nc21 ; C[Si]1(C)c2ccccc2-c2c(-c3ccc(-c4cccc(-c5ccccc5)c4)cc3)nc(-c3cccc(-c4cccc(-c5ccc(-c6ccccc6)cc5)c4)c3)nc21]

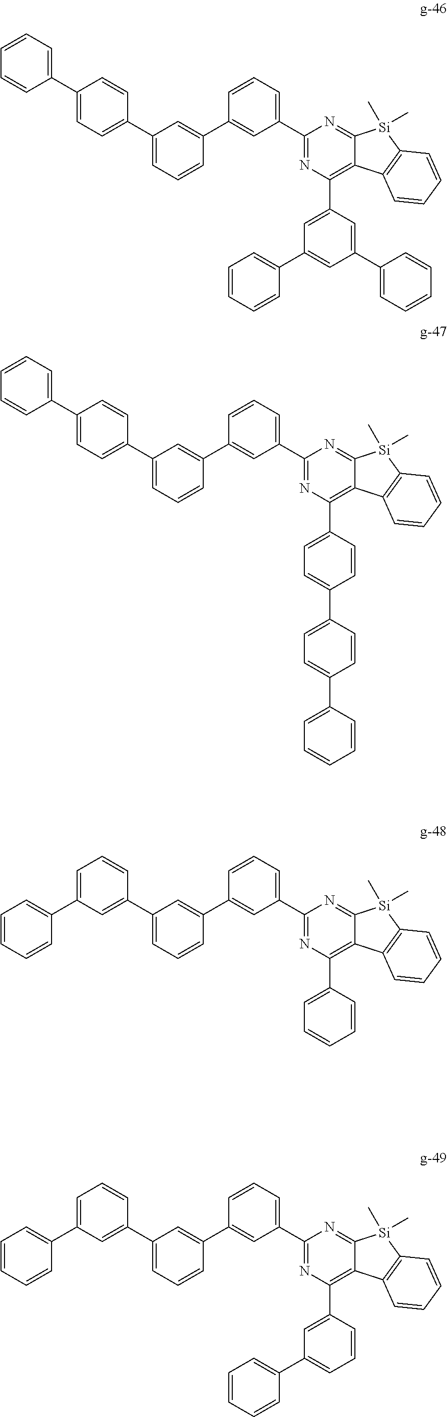 [SMILES: C[Si]1(C)c2ccccc2-c2c(-c3cc(-c4ccccc4)cc(-c4ccccc4)c3)nc(-c3cccc(-c4cccc(-c5ccc(-c6ccccc6)cc5)c4)c3)nc21 ; C[Si]1(C)c2ccccc2-c2c(-c3ccc(-c4ccc(-c5ccccc5)cc4)cc3)nc(-c3cccc(-c4cccc(-c5ccc(-c6ccccc6)cc5)c4)c3)nc21 ; C[Si]1(C)c2ccccc2-c2c(-c3ccccc3)nc(-c3cccc(-c4cccc(-c5cccc(-c6ccccc6)c5)c4)c3)nc21 ; C[Si]1(C)c2ccccc2-c2c(-c3cccc(-c4ccccc4)c3)nc(-c3cccc(-c4cccc(-c5cccc(-c6ccccc6)c5)c4)c3)nc21]

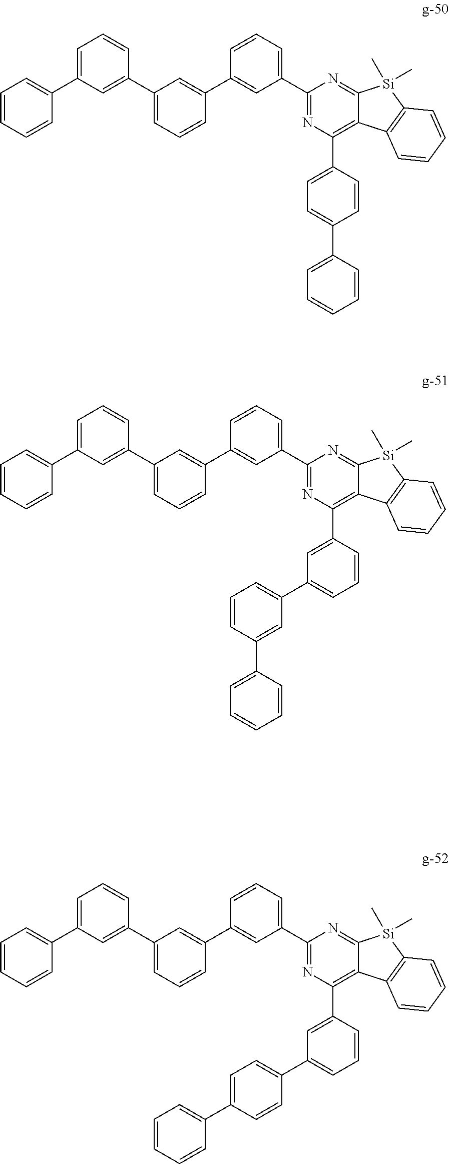 [SMILES: C[Si]1(C)c2ccccc2-c2c(-c3ccc(-c4ccccc4)cc3)nc(-c3cccc(-c4cccc(-c5cccc(-c6ccccc6)c5)c4)c3)nc21 ; C[Si]1(C)c2ccccc2-c2c(-c3cccc(-c4cccc(-c5ccccc5)c4)c3)nc(-c3cccc(-c4cccc(-c5cccc(-c6ccccc6)c5)c4)c3)nc21 ; C[Si]1(C)c2ccccc2-c2c(-c3cccc(-c4ccc(-c5ccccc5)cc4)c3)nc(-c3cccc(-c4cccc(-c5cccc(-c6ccccc6)c5)c4)c3)nc21]

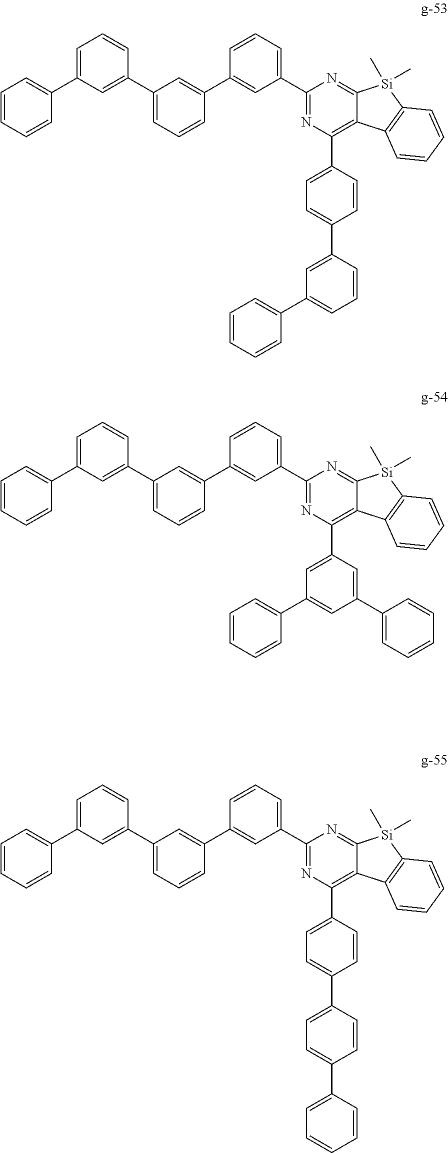 [SMILES: C[Si]1(C)c2ccccc2-c2c(-c3ccc(-c4cccc(-c5ccccc5)c4)cc3)nc(-c3cccc(-c4cccc(-c5cccc(-c6ccccc6)c5)c4)c3)nc21 ; C[Si]1(C)c2ccccc2-c2c(-c3cc(-c4ccccc4)cc(-c4ccccc4)c3)nc(-c3cccc(-c4cccc(-c5cccc(-c6ccccc6)c5)c4)c3)nc21 ; C[Si]1(C)c2ccccc2-c2c(-c3ccc(-c4ccc(-c5ccccc5)cc4)cc3)nc(-c3cccc(-c4cccc(-c5cccc(-c6ccccc6)c5)c4)c3)nc21]

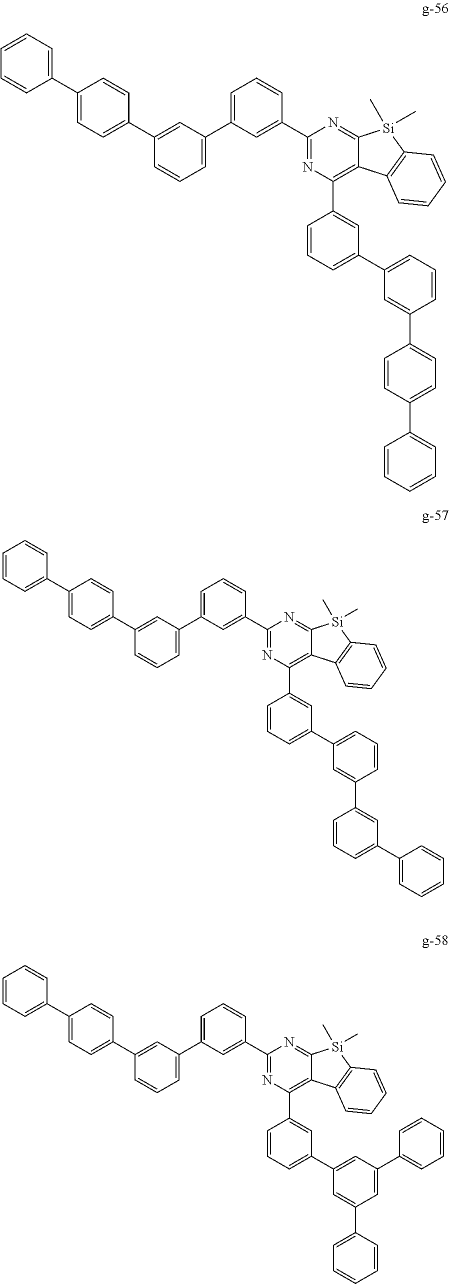 [SMILES: C[Si]1(C)c2ccccc2-c2c(-c3cccc(-c4cccc(-c5ccc(-c6ccccc6)cc5)c4)c3)nc(-c3cccc(-c4cccc(-c5ccc(-c6ccccc6)cc5)c4)c3)nc21 ; C[Si]1(C)c2ccccc2-c2c(-c3cccc(-c4cccc(-c5cccc(-c6ccccc6)c5)c4)c3)nc(-c3cccc(-c4cccc(-c5ccc(-c6ccccc6)cc5)c4)c3)nc21 ; C[Si]1(C)c2ccccc2-c2c(-c3cccc(-c4cc(-c5ccccc5)cc(-c5ccccc5)c4)c3)nc(-c3cccc(-c4cccc(-c5ccc(-c6ccccc6)cc5)c4)c3)nc21]

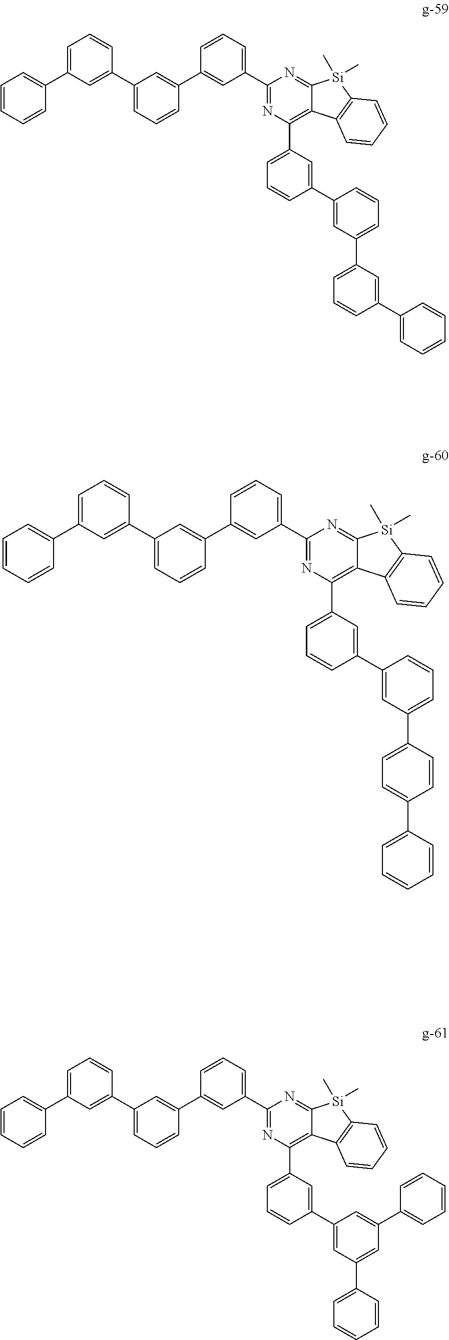 [SMILES: C[Si]1(C)c2ccccc2-c2c(-c3cccc(-c4cccc(-c5cccc(-c6ccccc6)c5)c4)c3)nc(-c3cccc(-c4cccc(-c5cccc(-c6ccccc6)c5)c4)c3)nc21 ; C[Si]1(C)c2ccccc2-c2c(-c3cccc(-c4cccc(-c5ccc(-c6ccccc6)cc5)c4)c3)nc(-c3cccc(-c4cccc(-c5cccc(-c6ccccc6)c5)c4)c3)nc21 ; C[Si]1(C)c2ccccc2-c2c(-c3cccc(-c4cc(-c5ccccc5)cc(-c5ccccc5)c4)c3)nc(-c3cccc(-c4cccc(-c5cccc(-c6ccccc6)c5)c4)c3)nc21]

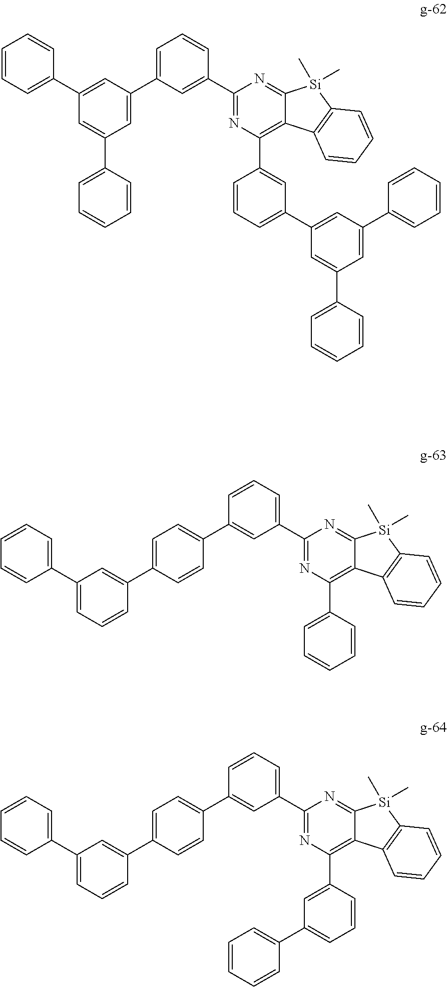 [SMILES: C[Si]1(C)c2ccccc2-c2c(-c3cccc(-c4cc(-c5ccccc5)cc(-c5ccccc5)c4)c3)nc(-c3cccc(-c4cc(-c5ccccc5)cc(-c5ccccc5)c4)c3)nc21 ; C[Si]1(C)c2ccccc2-c2c(-c3ccccc3)nc(-c3cccc(-c4ccc(-c5cccc(-c6ccccc6)c5)cc4)c3)nc21 ; C[Si]1(C)c2ccccc2-c2c(-c3cccc(-c4ccccc4)c3)nc(-c3cccc(-c4ccc(-c5cccc(-c6ccccc6)c5)cc4)c3)nc21]

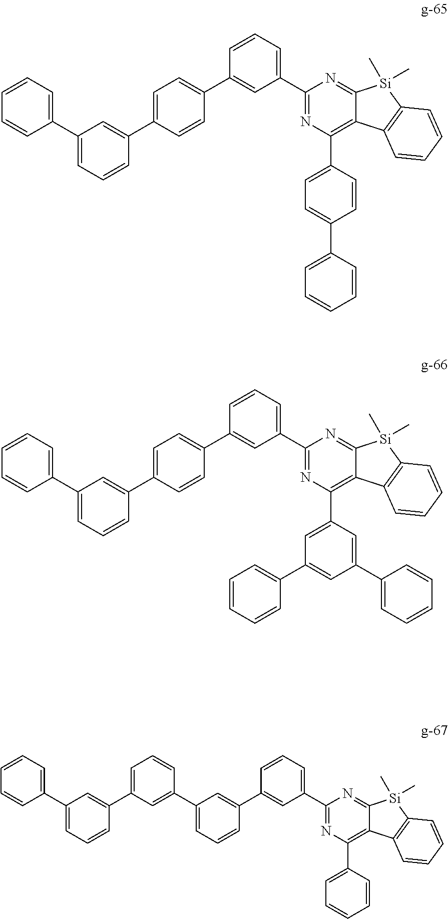 [SMILES: C[Si]1(C)c2ccccc2-c2c(-c3ccc(-c4ccccc4)cc3)nc(-c3cccc(-c4ccc(-c5cccc(-c6ccccc6)c5)cc4)c3)nc21 ; C[Si]1(C)c2ccccc2-c2c(-c3cc(-c4ccccc4)cc(-c4ccccc4)c3)nc(-c3cccc(-c4ccc(-c5cccc(-c6ccccc6)c5)cc4)c3)nc21 ; C[Si]1(C)c2ccccc2-c2c(-c3ccccc3)nc(-c3cccc(-c4cccc(-c5cccc(-c6cccc(-c7ccccc7)c6)c5)c4)c3)nc21]

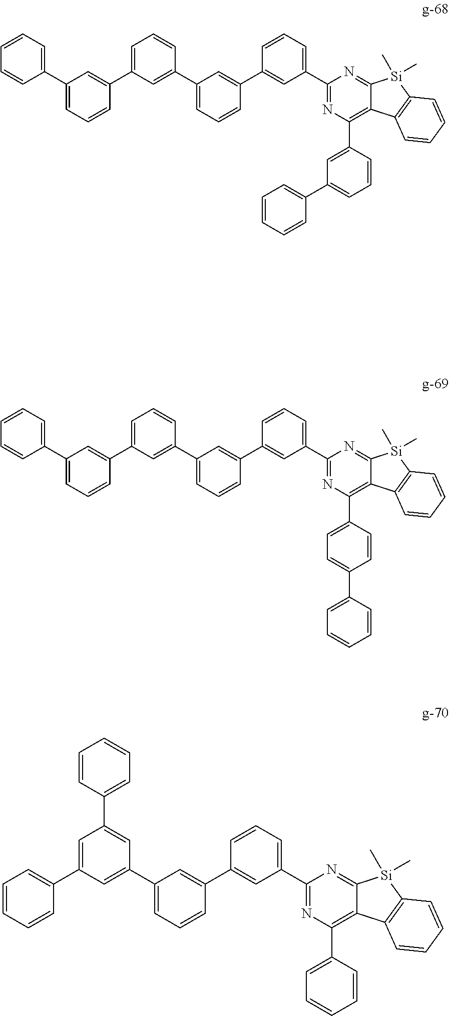 [SMILES: C[Si]1(C)c2ccccc2-c2c(-c3cccc(-c4ccccc4)c3)nc(-c3cccc(-c4cccc(-c5cccc(-c6cccc(-c7ccccc7)c6)c5)c4)c3)nc21 ; C[Si]1(C)c2ccccc2-c2c(-c3ccc(-c4ccccc4)cc3)nc(-c3cccc(-c4cccc(-c5cccc(-c6cccc(-c7ccccc7)c6)c5)c4)c3)nc21 ; C[Si]1(C)c2ccccc2-c2c(-c3ccccc3)nc(-c3cccc(-c4cccc(-c5cc(-c6ccccc6)cc(-c6ccccc6)c5)c4)c3)nc21]

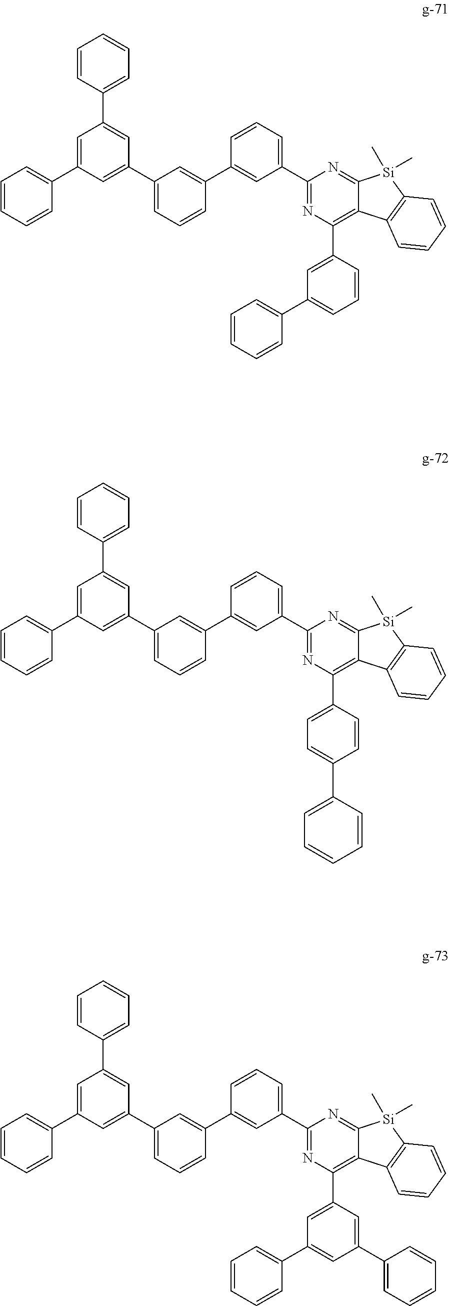 [SMILES: C[Si]1(C)c2ccccc2-c2c(-c3cccc(-c4ccccc4)c3)nc(-c3cccc(-c4cccc(-c5cc(-c6ccccc6)cc(-c6ccccc6)c5)c4)c3)nc21 ; C[Si]1(C)c2ccccc2-c2c(-c3ccc(-c4ccccc4)cc3)nc(-c3cccc(-c4cccc(-c5cc(-c6ccccc6)cc(-c6ccccc6)c5)c4)c3)nc21 ; C[Si]1(C)c2ccccc2-c2c(-c3cc(-c4ccccc4)cc(-c4ccccc4)c3)nc(-c3cccc(-c4cccc(-c5cc(-c6ccccc6)cc(-c6ccccc6)c5)c4)c3)nc21]

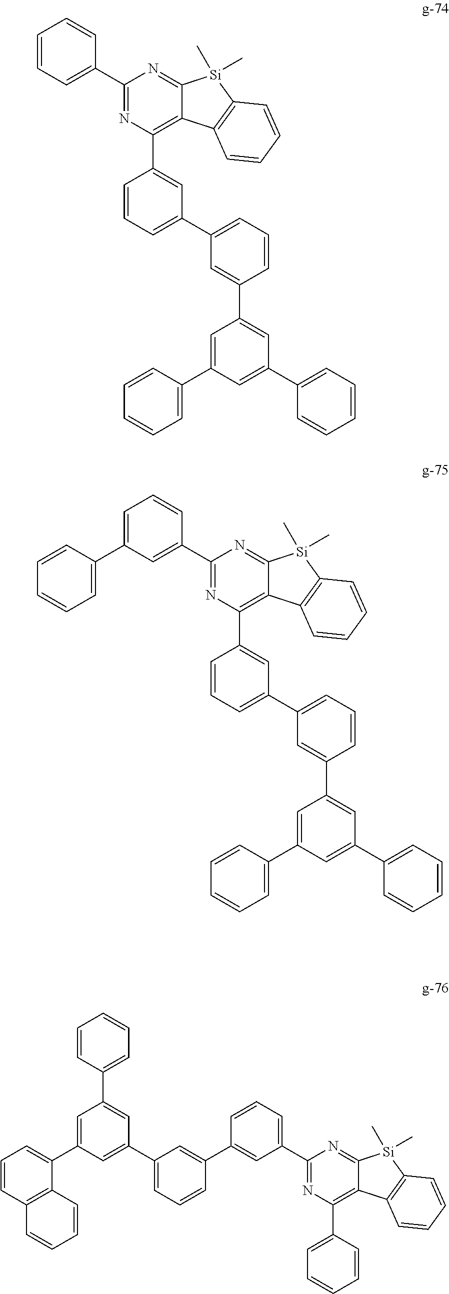 [SMILES: C[Si]1(C)c2ccccc2-c2c(-c3cccc(-c4cccc(-c5cc(-c6ccccc6)cc(-c6ccccc6)c5)c4)c3)nc(-c3ccccc3)nc21 ; C[Si]1(C)c2ccccc2-c2c(-c3cccc(-c4cccc(-c5cc(-c6ccccc6)cc(-c6ccccc6)c5)c4)c3)nc(-c3cccc(-c4ccccc4)c3)nc21 ; C[Si]1(C)c2ccccc2-c2c(-c3ccccc3)nc(-c3cccc(-c4cccc(-c5cc(-c6ccccc6)cc(-c6cccc7ccccc67)c5)c4)c3)nc21]

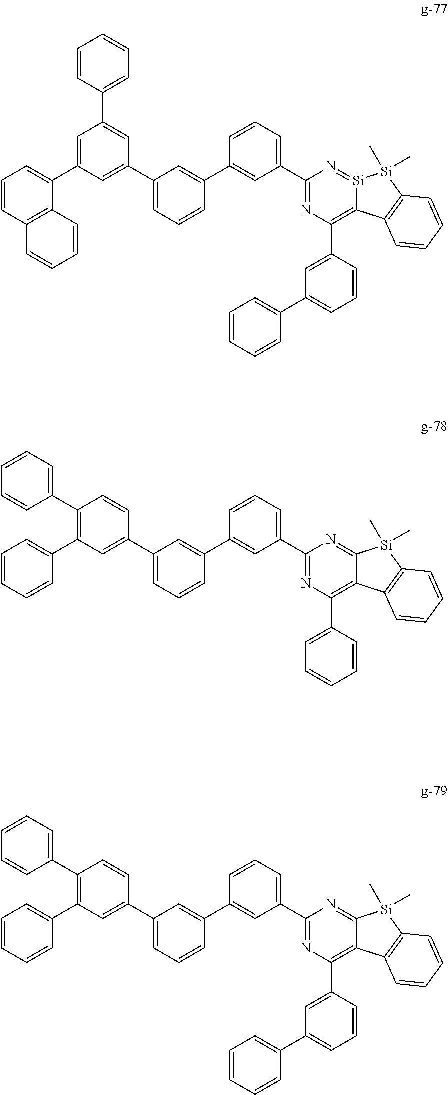 [SMILES: C[Si]1(C)c2ccccc2-c2c(-c3cccc(-c4ccccc4)c3)nc(-c3cccc(-c4cccc(-c5cc(-c6ccccc6)cc(-c6cccc7ccccc67)c5)c4)c3)n[si]21 ; C[Si]1(C)c2ccccc2-c2c(-c3ccccc3)nc(-c3cccc(-c4cccc(-c5ccc(-c6ccccc6)c(-c6ccccc6)c5)c4)c3)nc21 ; C[Si]1(C)c2ccccc2-c2c(-c3cccc(-c4ccccc4)c3)nc(-c3cccc(-c4cccc(-c5ccc(-c6ccccc6)c(-c6ccccc6)c5)c4)c3)nc21]

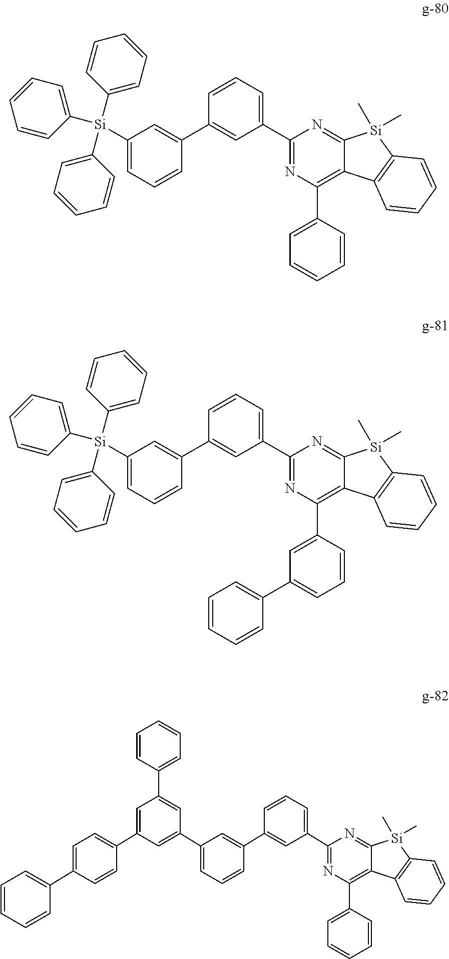 [SMILES: C[Si]1(C)c2ccccc2-c2c(-c3ccccc3)nc(-c3cccc(-c4cccc([Si](c5ccccc5)(c5ccccc5)c5ccccc5)c4)c3)nc21 ; C[Si]1(C)c2ccccc2-c2c(-c3cccc(-c4ccccc4)c3)nc(-c3cccc(-c4cccc([Si](c5ccccc5)(c5ccccc5)c5ccccc5)c4)c3)nc21 ; C[Si]1(C)c2ccccc2-c2c(-c3ccccc3)nc(-c3cccc(-c4cccc(-c5cc(-c6ccccc6)cc(-c6ccc(-c7ccccc7)cc6)c5)c4)c3)nc21]

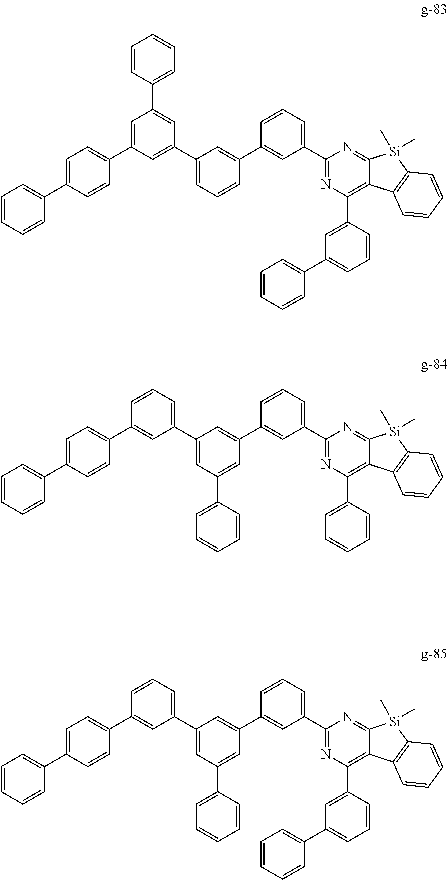 [SMILES: C[Si]1(C)c2ccccc2-c2c(-c3cccc(-c4ccccc4)c3)nc(-c3cccc(-c4cccc(-c5cc(-c6ccccc6)cc(-c6ccc(-c7ccccc7)cc6)c5)c4)c3)nc21 ; C[Si]1(C)c2ccccc2-c2c(-c3ccccc3)nc(-c3cccc(-c4cc(-c5ccccc5)cc(-c5cccc(-c6ccc(-c7ccccc7)cc6)c5)c4)c3)nc21 ; C[Si]1(C)c2ccccc2-c2c(-c3cccc(-c4ccccc4)c3)nc(-c3cccc(-c4cc(-c5ccccc5)cc(-c5cccc(-c6ccc(-c7ccccc7)cc6)c5)c4)c3)nc21]

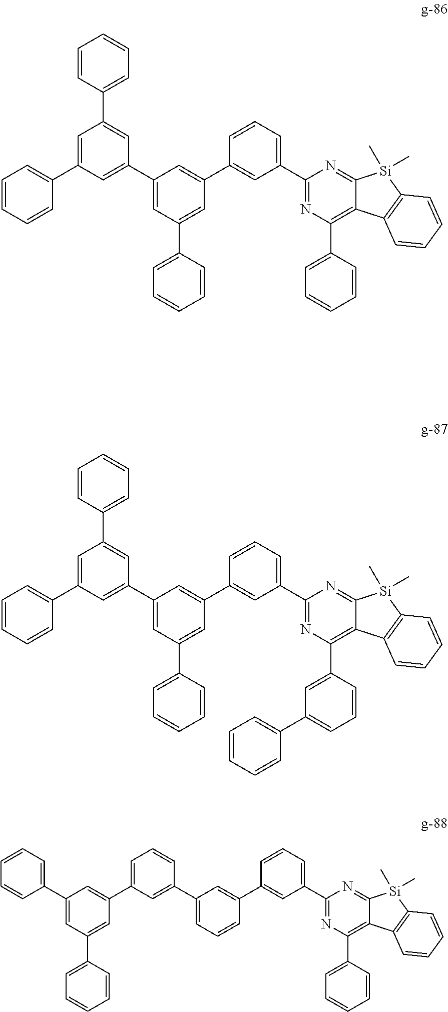 [SMILES: C[Si]1(C)c2ccccc2-c2c(-c3ccccc3)nc(-c3cccc(-c4cc(-c5ccccc5)cc(-c5cc(-c6ccccc6)cc(-c6ccccc6)c5)c4)c3)nc21 ; C[Si]1(C)c2ccccc2-c2c(-c3cccc(-c4ccccc4)c3)nc(-c3cccc(-c4cc(-c5ccccc5)cc(-c5cc(-c6ccccc6)cc(-c6ccccc6)c5)c4)c3)nc21 ; C[Si]1(C)c2ccccc2-c2c(-c3ccccc3)nc(-c3cccc(-c4cccc(-c5cccc(-c6cc(-c7ccccc7)cc(-c7ccccc7)c6)c5)c4)c3)nc21]

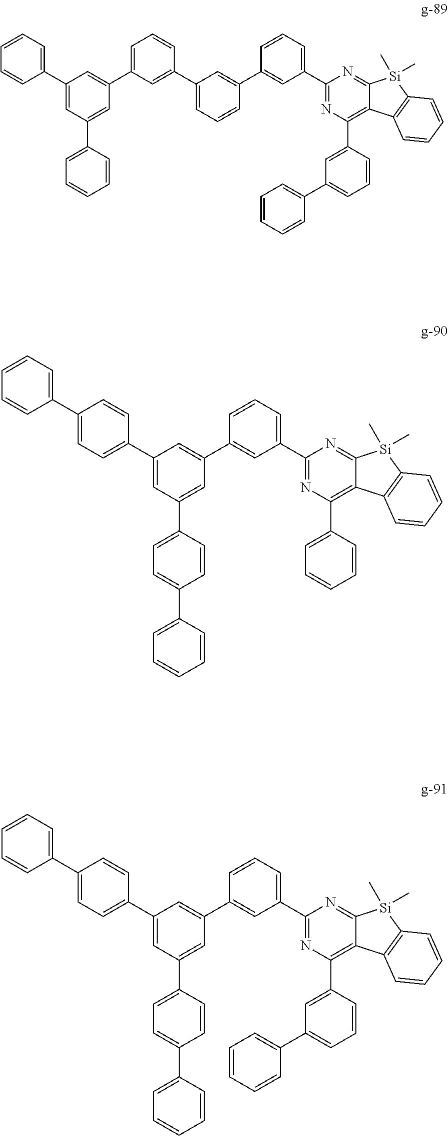 [SMILES: C[Si]1(C)c2ccccc2-c2c(-c3cccc(-c4ccccc4)c3)nc(-c3cccc(-c4cccc(-c5cccc(-c6cc(-c7ccccc7)cc(-c7ccccc7)c6)c5)c4)c3)nc21 ; C[Si]1(C)c2ccccc2-c2c(-c3ccccc3)nc(-c3cccc(-c4cc(-c5ccc(-c6ccccc6)cc5)cc(-c5ccc(-c6ccccc6)cc5)c4)c3)nc21 ; C[Si]1(C)c2ccccc2-c2c(-c3cccc(-c4ccccc4)c3)nc(-c3cccc(-c4cc(-c5ccc(-c6ccccc6)cc5)cc(-c5ccc(-c6ccccc6)cc5)c4)c3)nc21]

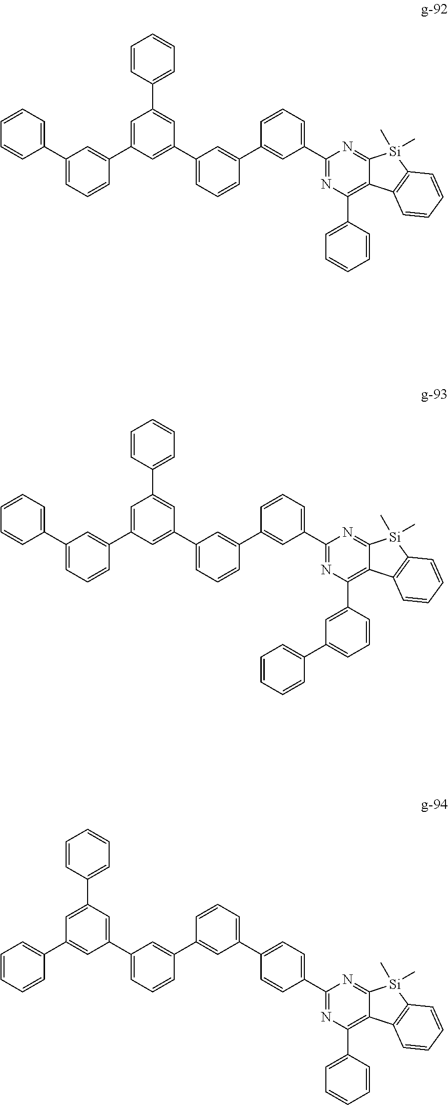 [SMILES: C[Si]1(C)c2ccccc2-c2c(-c3ccccc3)nc(-c3cccc(-c4cccc(-c5cc(-c6ccccc6)cc(-c6cccc(-c7ccccc7)c6)c5)c4)c3)nc21 ; C[Si]1(C)c2ccccc2-c2c(-c3cccc(-c4ccccc4)c3)nc(-c3cccc(-c4cccc(-c5cc(-c6ccccc6)cc(-c6cccc(-c7ccccc7)c6)c5)c4)c3)nc21 ; C[Si]1(C)c2ccccc2-c2c(-c3ccccc3)nc(-c3ccc(-c4cccc(-c5cccc(-c6cc(-c7ccccc7)cc(-c7ccccc7)c6)c5)c4)cc3)nc21]

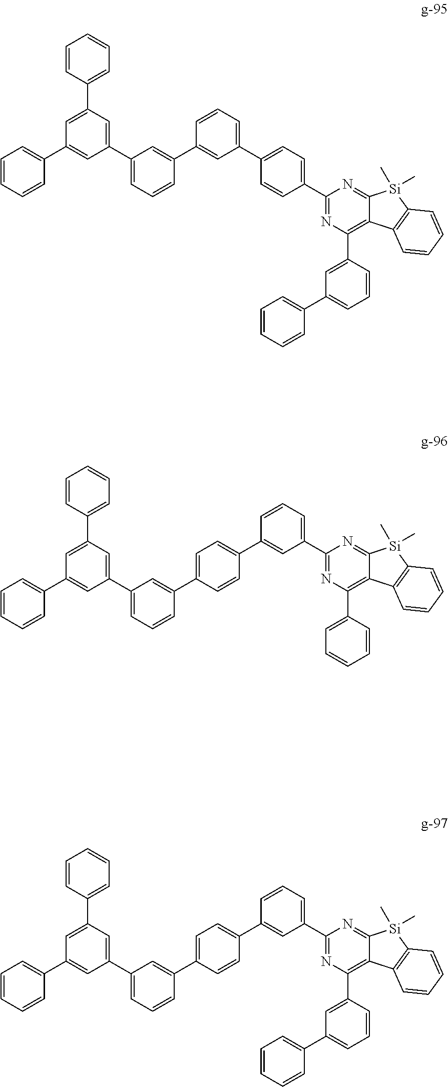 [SMILES: C[Si]1(C)c2ccccc2-c2c(-c3cccc(-c4ccccc4)c3)nc(-c3ccc(-c4cccc(-c5cccc(-c6cc(-c7ccccc7)cc(-c7ccccc7)c6)c5)c4)cc3)nc21 ; C[Si]1(C)c2ccccc2-c2c(-c3ccccc3)nc(-c3cccc(-c4ccc(-c5cccc(-c6cc(-c7ccccc7)cc(-c7ccccc7)c6)c5)cc4)c3)nc21 ; C[Si]1(C)c2ccccc2-c2c(-c3cccc(-c4ccccc4)c3)nc(-c3cccc(-c4ccc(-c5cccc(-c6cc(-c7ccccc7)cc(-c7ccccc7)c6)c5)cc4)c3)nc21]

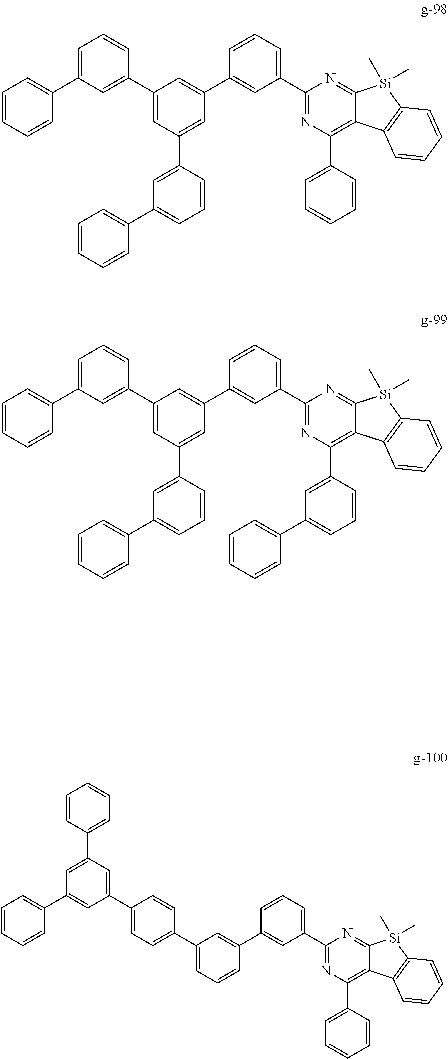 [SMILES: C[Si]1(C)c2ccccc2-c2c(-c3ccccc3)nc(-c3cccc(-c4cc(-c5cccc(-c6ccccc6)c5)cc(-c5cccc(-c6ccccc6)c5)c4)c3)nc21 ; C[Si]1(C)c2ccccc2-c2c(-c3cccc(-c4ccccc4)c3)nc(-c3cccc(-c4cc(-c5cccc(-c6ccccc6)c5)cc(-c5cccc(-c6ccccc6)c5)c4)c3)nc21 ; C[Si]1(C)c2ccccc2-c2c(-c3ccccc3)nc(-c3cccc(-c4cccc(-c5ccc(-c6cc(-c7ccccc7)cc(-c7ccccc7)c6)cc5)c4)c3)nc21]

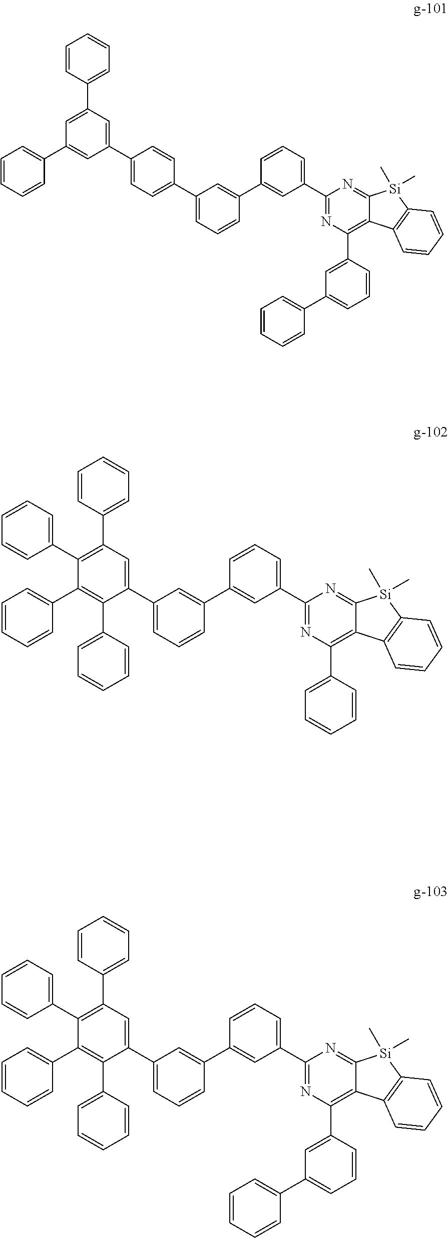 [SMILES: C[Si]1(C)c2ccccc2-c2c(-c3cccc(-c4ccccc4)c3)nc(-c3cccc(-c4cccc(-c5ccc(-c6cc(-c7ccccc7)cc(-c7ccccc7)c6)cc5)c4)c3)nc21 ; C[Si]1(C)c2ccccc2-c2c(-c3ccccc3)nc(-c3cccc(-c4cccc(-c5cc(-c6ccccc6)c(-c6ccccc6)c(-c6ccccc6)c5-c5ccccc5)c4)c3)nc21 ; C[Si]1(C)c2ccccc2-c2c(-c3cccc(-c4ccccc4)c3)nc(-c3cccc(-c4cccc(-c5cc(-c6ccccc6)c(-c6ccccc6)c(-c6ccccc6)c5-c5ccccc5)c4)c3)nc21]

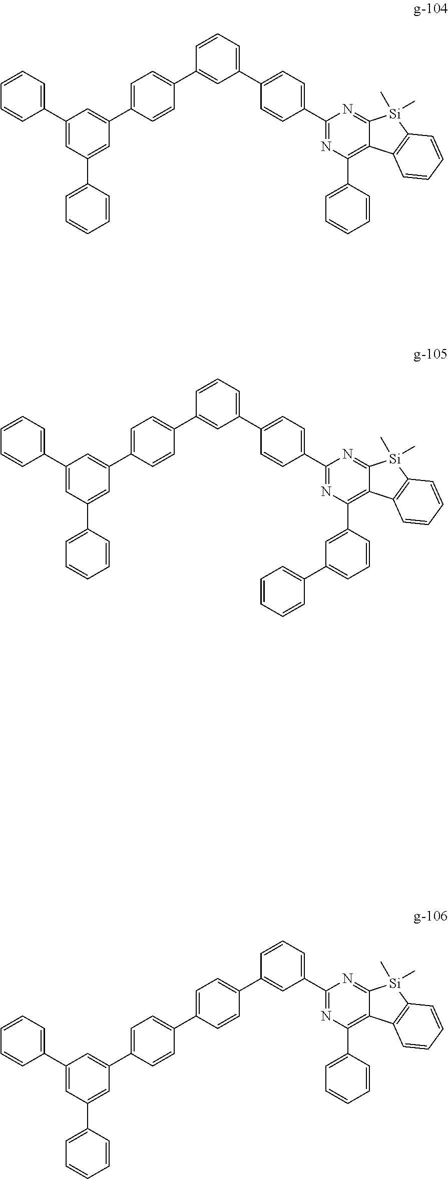 [SMILES: C[Si]1(C)c2ccccc2-c2c(-c3ccccc3)nc(-c3ccc(-c4cccc(-c5ccc(-c6cc(-c7ccccc7)cc(-c7ccccc7)c6)cc5)c4)cc3)nc21 ; C[Si]1(C)c2ccccc2-c2c(-c3cccc(-c4ccccc4)c3)nc(-c3ccc(-c4cccc(-c5ccc(-c6cc(-c7ccccc7)cc(-c7ccccc7)c6)cc5)c4)cc3)nc21 ; C[Si]1(C)c2ccccc2-c2c(-c3ccccc3)nc(-c3cccc(-c4ccc(-c5ccc(-c6cc(-c7ccccc7)cc(-c7ccccc7)c6)cc5)cc4)c3)nc21]

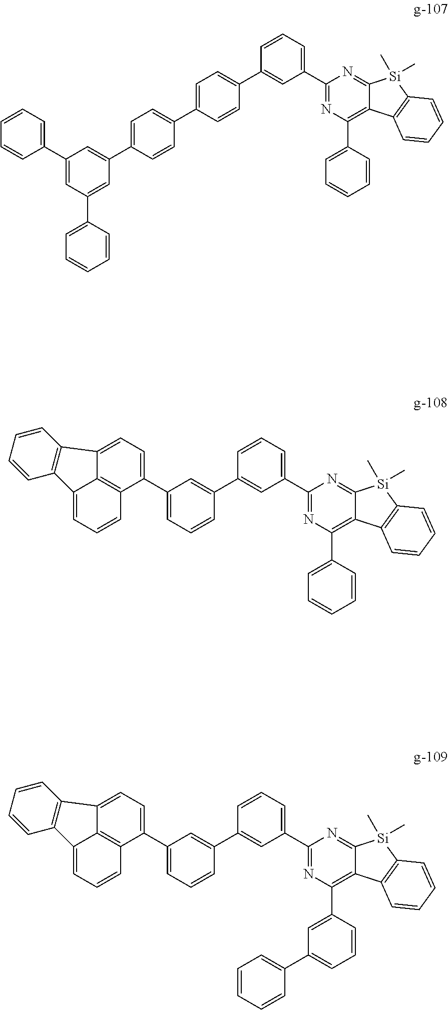 [SMILES: C[Si]1(C)c2ccccc2-c2c(-c3ccccc3)nc(-c3cccc(-c4ccc(-c5ccc(-c6cc(-c7ccccc7)cc(-c7ccccc7)c6)cc5)cc4)c3)nc21 ; C[Si]1(C)c2ccccc2-c2c(-c3ccccc3)nc(-c3cccc(-c4cccc(-c5ccc6c7c(cccc57)-c5ccccc5-6)c4)c3)nc21 ; C[Si]1(C)c2ccccc2-c2c(-c3cccc(-c4ccccc4)c3)nc(-c3cccc(-c4cccc(-c5ccc6c7c(cccc57)-c5ccccc5-6)c4)c3)nc21]

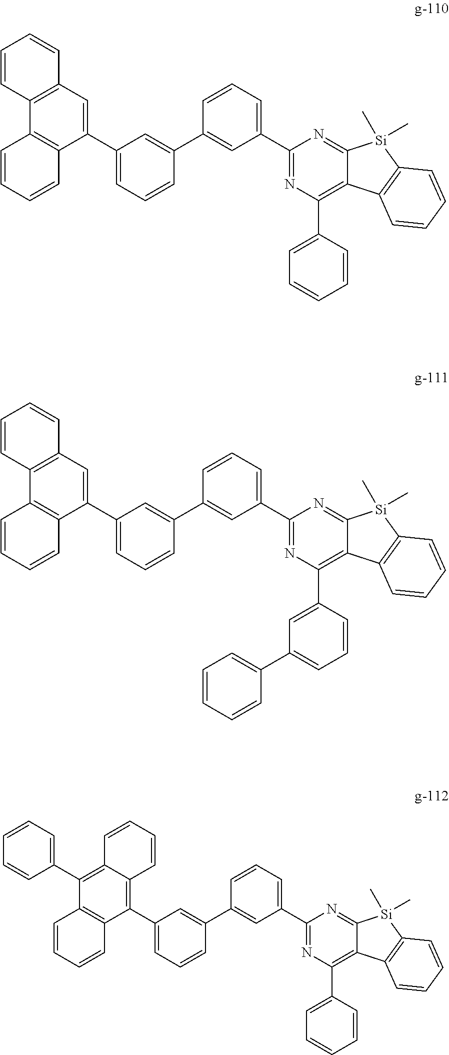 [SMILES: C[Si]1(C)c2ccccc2-c2c(-c3ccccc3)nc(-c3cccc(-c4cccc(-c5cc6ccccc6c6ccccc56)c4)c3)nc21 ; C[Si]1(C)c2ccccc2-c2c(-c3cccc(-c4ccccc4)c3)nc(-c3cccc(-c4cccc(-c5cc6ccccc6c6ccccc56)c4)c3)nc21 ; C[Si]1(C)c2ccccc2-c2c(-c3ccccc3)nc(-c3cccc(-c4cccc(-c5c6ccccc6c(-c6ccccc6)c6ccccc56)c4)c3)nc21]

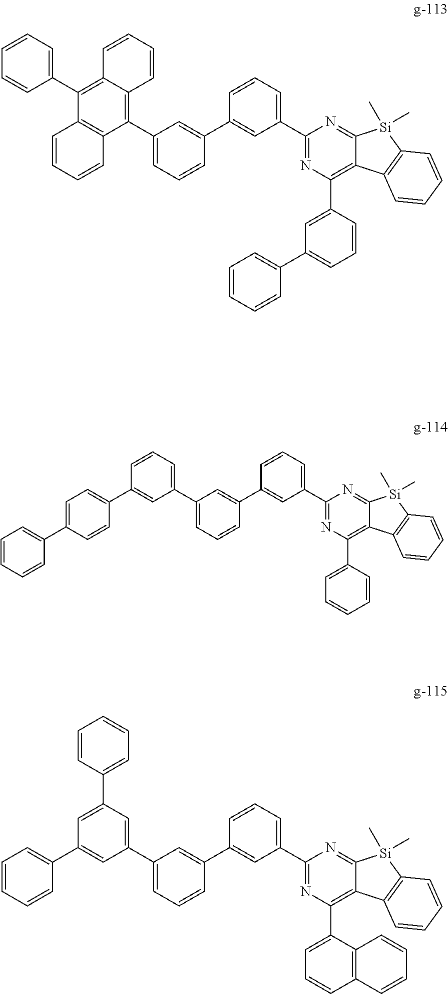 [SMILES: C[Si]1(C)c2ccccc2-c2c(-c3cccc(-c4ccccc4)c3)nc(-c3cccc(-c4cccc(-c5c6ccccc6c(-c6ccccc6)c6ccccc56)c4)c3)nc21 ; C[Si]1(C)c2ccccc2-c2c(-c3ccccc3)nc(-c3cccc(-c4cccc(-c5cccc(-c6ccc(-c7ccccc7)cc6)c5)c4)c3)nc21 ; C[Si]1(C)c2ccccc2-c2c(-c3cccc4ccccc34)nc(-c3cccc(-c4cccc(-c5cc(-c6ccccc6)cc(-c6ccccc6)c5)c4)c3)nc21]

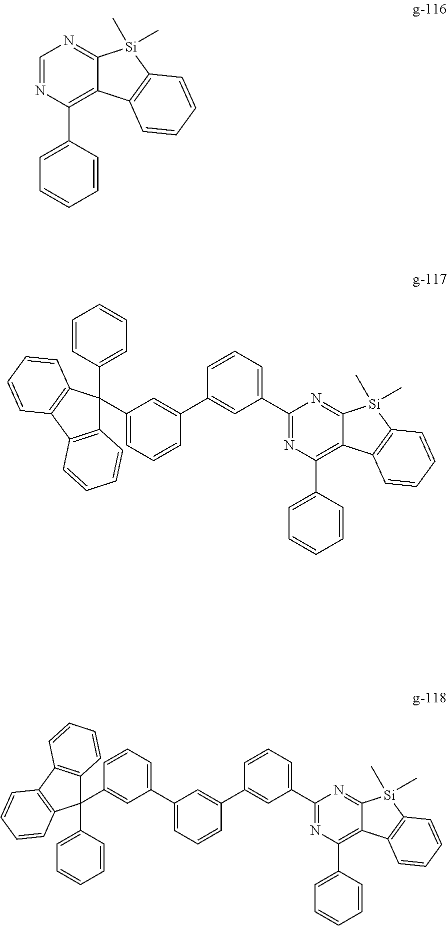 [SMILES: C[Si]1(C)c2ccccc2-c2c(-c3ccccc3)ncnc21 ; C[Si]1(C)c2ccccc2-c2c(-c3ccccc3)nc(-c3cccc(-c4cccc(C5(c6ccccc6)c6ccccc6-c6ccccc65)c4)c3)nc21 ; C[Si]1(C)c2ccccc2-c2c(-c3ccccc3)nc(-c3cccc(-c4cccc(-c5cccc(C6(c7ccccc7)c7ccccc7-c7ccccc76)c5)c4)c3)nc21]

Group of X₁=N-[(L₁)a1-(R₁)b1] in Formula 1-2

(L₁, a1, R₁ and b1 are described in the present specification)

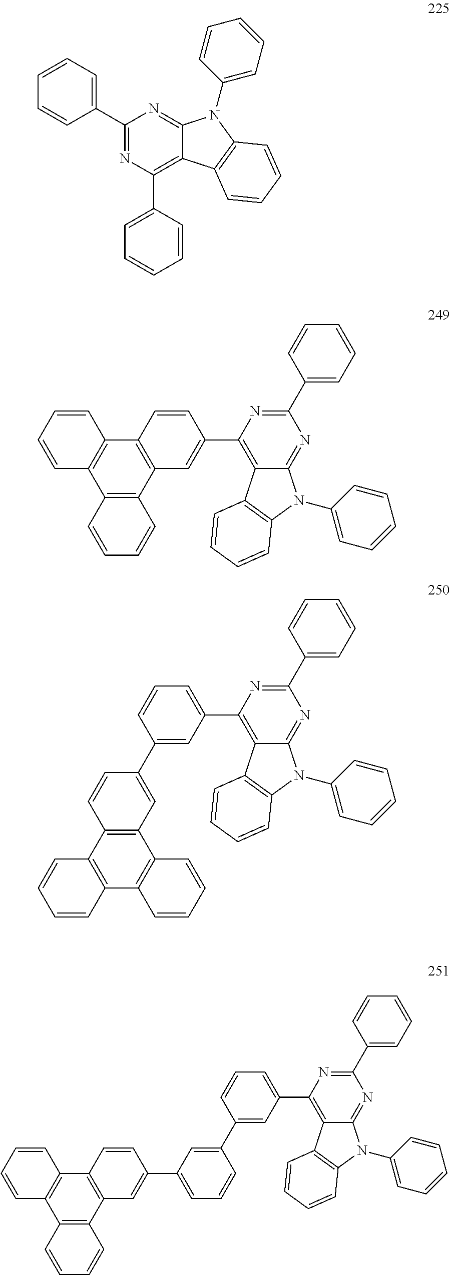 [SMILES: c1ccc(-c2nc(-c3ccccc3)c3c4ccccc4n(-c4ccccc4)c3n2)cc1 ; c1ccc(-c2nc(-c3ccc4c5ccccc5c5ccccc5c4c3)c3c4ccccc4n(-c4ccccc4)c3n2)cc1 ; c1ccc(-c2nc(-c3cccc(-c4ccc5c6ccccc6c6ccccc6c5c4)c3)c3c4ccccc4n(-c4ccccc4)c3n2)cc1 ; c1ccc(-c2nc(-c3cccc(-c4cccc(-c5ccc6c7ccccc7c7ccccc7c6c5)c4)c3)c3c4ccccc4n(-c4ccccc4)c3n2)cc1]

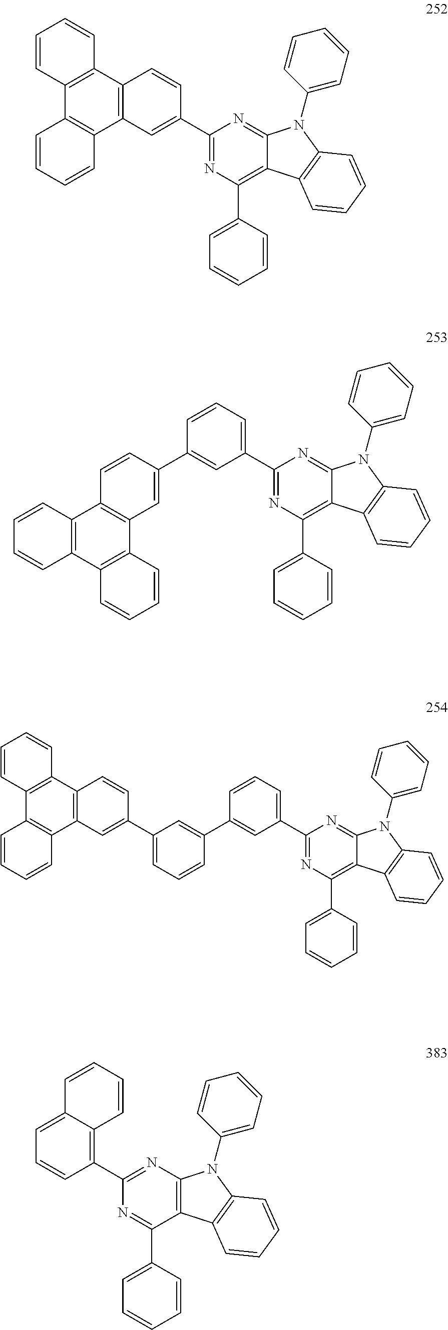 [SMILES: c1ccc(-c2nc(-c3ccc4c5ccccc5c5ccccc5c4c3)nc3c2c2ccccc2n3-c2ccccc2)cc1 ; c1ccc(-c2nc(-c3cccc(-c4ccc5c6ccccc6c6ccccc6c5c4)c3)nc3c2c2ccccc2n3-c2ccccc2)cc1 ; c1ccc(-c2nc(-c3cccc(-c4cccc(-c5ccc6c7ccccc7c7ccccc7c6c5)c4)c3)nc3c2c2ccccc2n3-c2ccccc2)cc1 ; c1ccc(-c2nc(-c3cccc4ccccc34)nc3c2c2ccccc2n3-c2ccccc2)cc1]

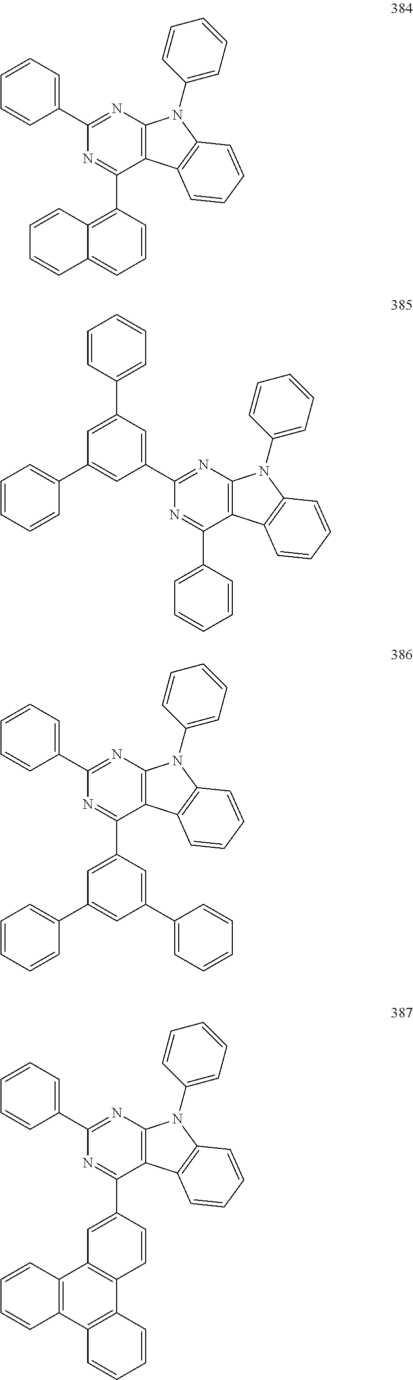

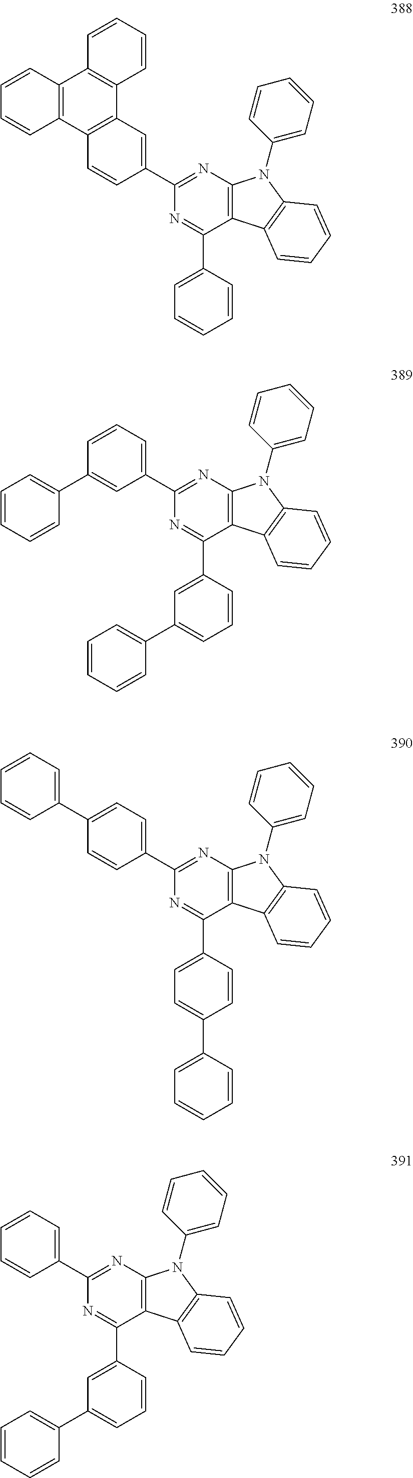 [SMILES: c1ccc(-c2nc(-c3ccc4c5ccccc5c5ccccc5c4c3)nc3c2c2ccccc2n3-c2ccccc2)cc1 ; c1ccc(-c2cccc(-c3nc(-c4cccc(-c5ccccc5)c4)c4c5ccccc5n(-c5ccccc5)c4n3)c2)cc1 ; c1ccc(-c2ccc(-c3nc(-c4ccc(-c5ccccc5)cc4)c4c5ccccc5n(-c5ccccc5)c4n3)cc2)cc1 ; c1ccc(-c2cccc(-c3nc(-c4ccccc4)nc4c3c3ccccc3n4-c3ccccc3)c2)cc1]

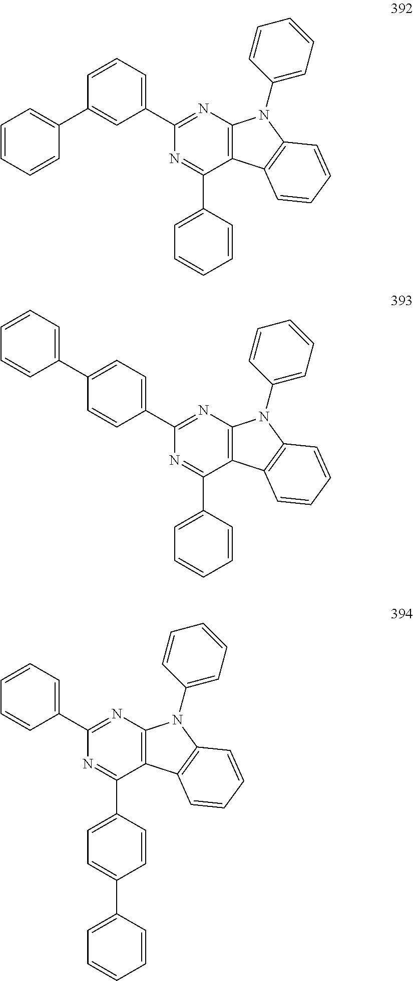

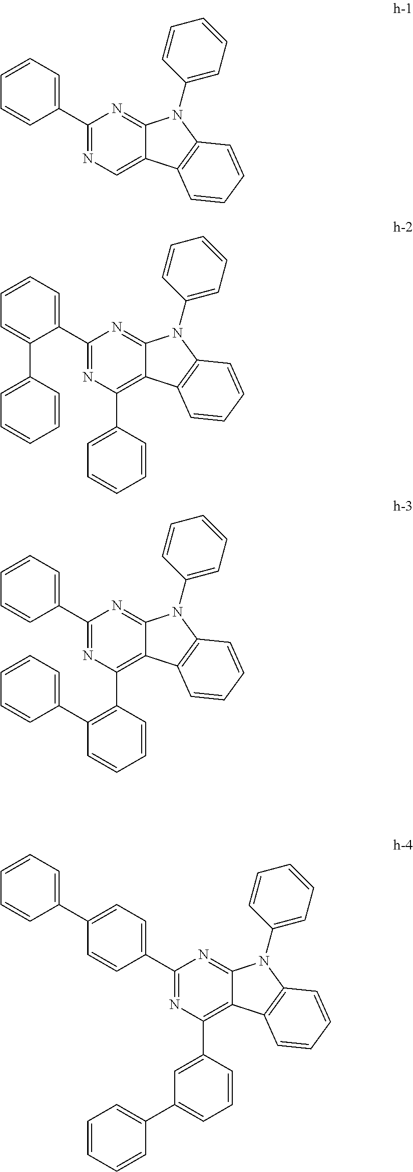

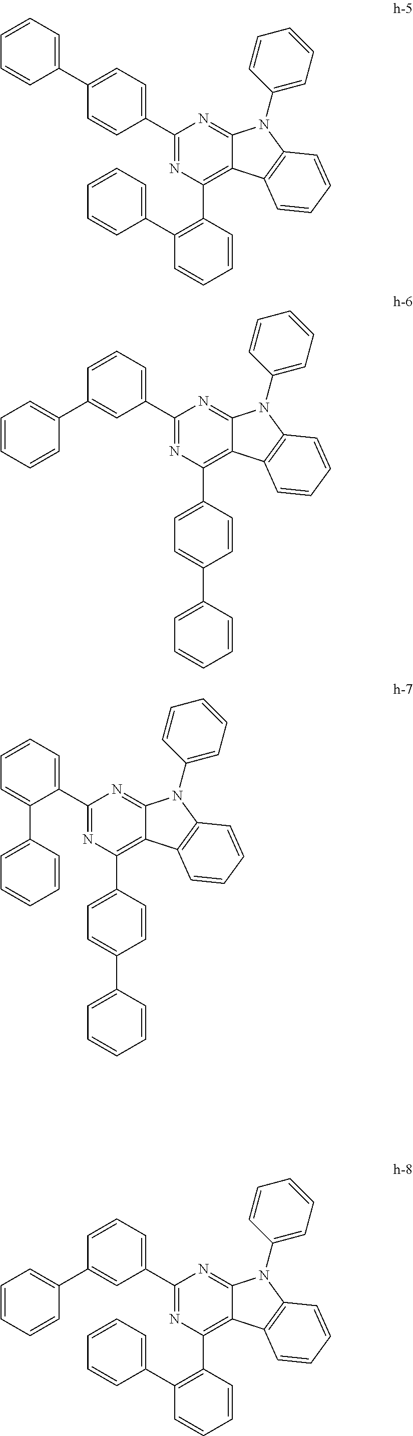

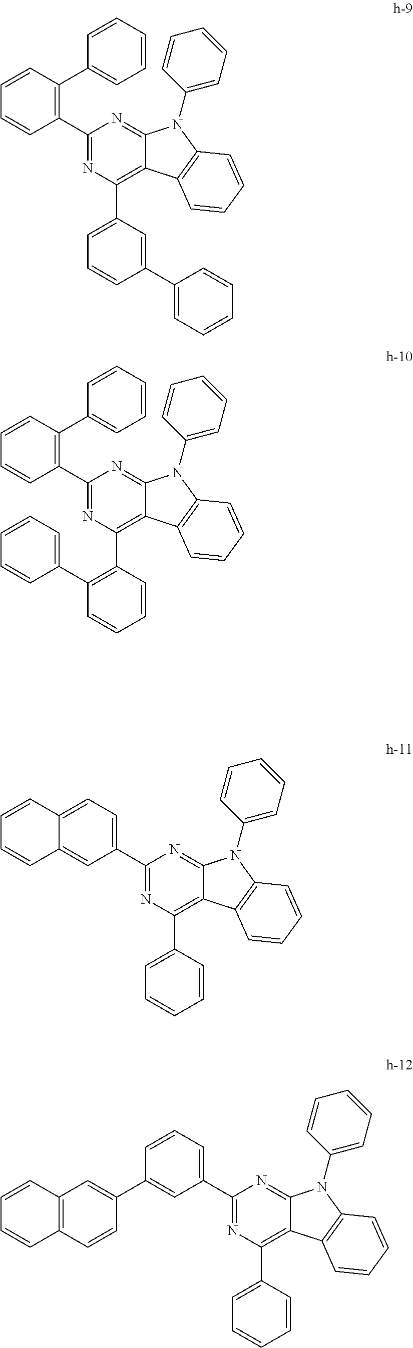

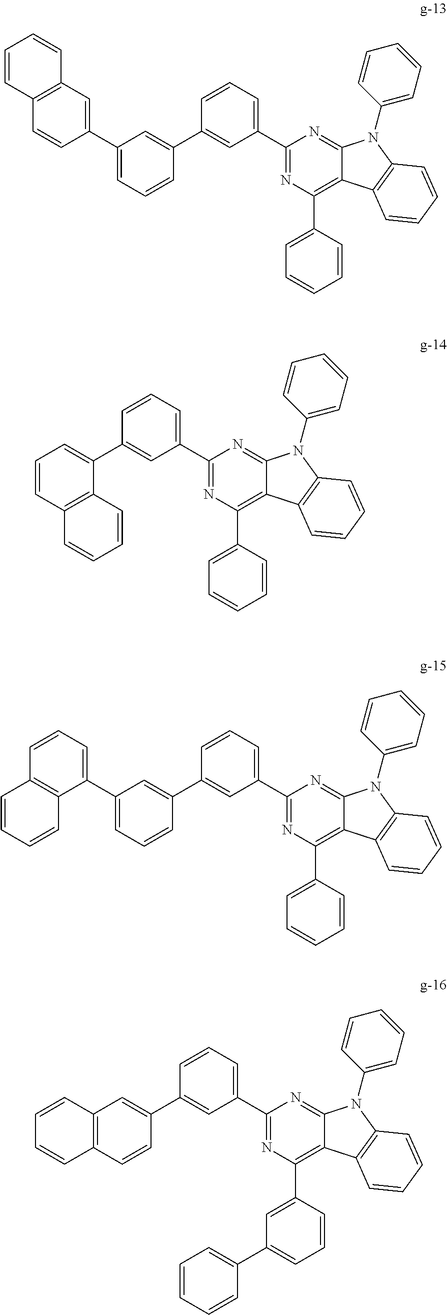 [SMILES: c1ccc(-c2nc(-c3cccc(-c4cccc(-c5ccc6ccccc6c5)c4)c3)nc3c2c2ccccc2n3-c2ccccc2)cc1 ; c1ccc(-c2nc(-c3cccc(-c4cccc5ccccc45)c3)nc3c2c2ccccc2n3-c2ccccc2)cc1 ; c1ccc(-c2nc(-c3cccc(-c4cccc(-c5cccc6ccccc56)c4)c3)nc3c2c2ccccc2n3-c2ccccc2)cc1 ; c1ccc(-c2cccc(-c3nc(-c4cccc(-c5ccc6ccccc6c5)c4)nc4c3c3ccccc3n4-c3ccccc3)c2)cc1]

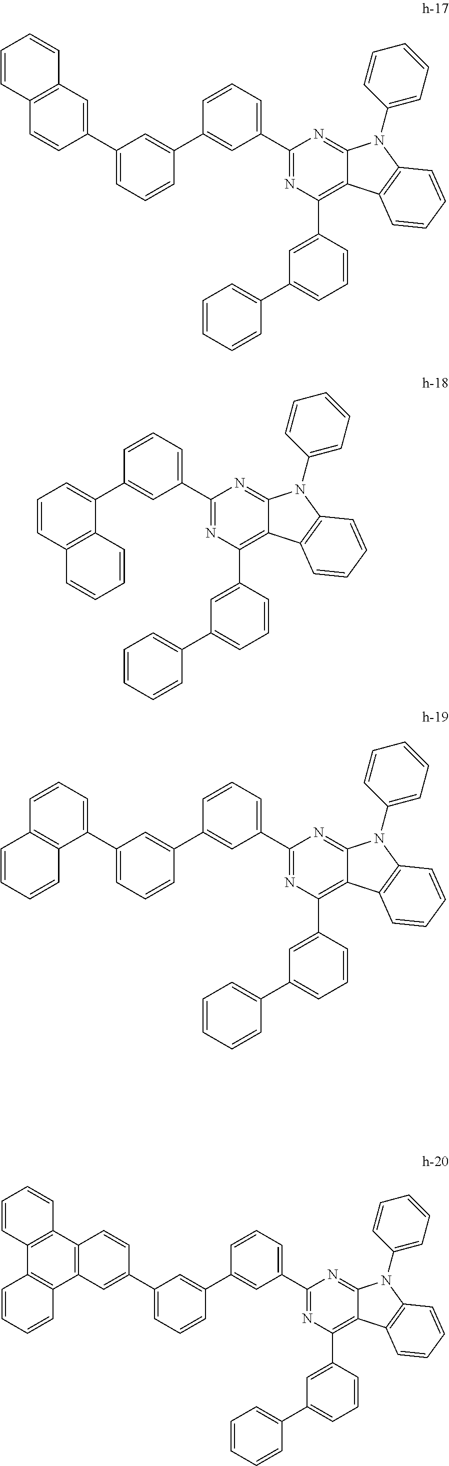 [SMILES: c1ccc(-c2cccc(-c3nc(-c4cccc(-c5cccc(-c6ccc7ccccc7c6)c5)c4)nc4c3c3ccccc3n4-c3ccccc3)c2)cc1 ; c1ccc(-c2cccc(-c3nc(-c4cccc(-c5cccc6ccccc56)c4)nc4c3c3ccccc3n4-c3ccccc3)c2)cc1 ; c1ccc(-c2cccc(-c3nc(-c4cccc(-c5cccc(-c6cccc7ccccc67)c5)c4)nc4c3c3ccccc3n4-c3ccccc3)c2)cc1 ; c1ccc(-c2cccc(-c3nc(-c4cccc(-c5cccc(-c6ccc7c8ccccc8c8ccccc8c7c6)c5)c4)nc4c3c3ccccc3n4-c3ccccc3)c2)cc1]

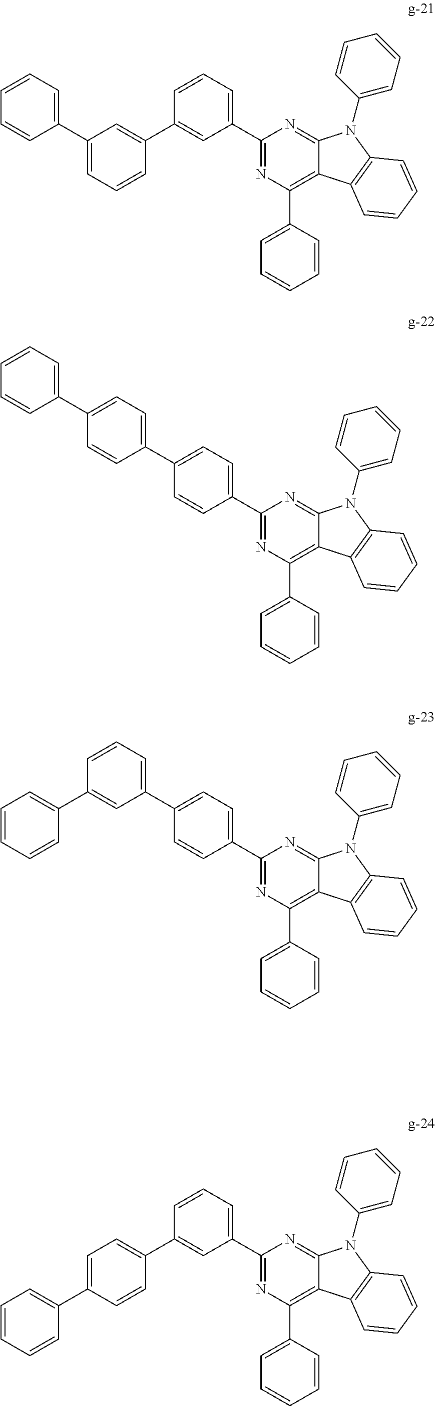 [SMILES: c1ccc(-c2cccc(-c3cccc(-c4nc(-c5ccccc5)c5c6ccccc6n(-c6ccccc6)c5n4)c3)c2)cc1 ; c1ccc(-c2ccc(-c3ccc(-c4nc(-c5ccccc5)c5c6ccccc6n(-c6ccccc6)c5n4)cc3)cc2)cc1 ; c1ccc(-c2cccc(-c3ccc(-c4nc(-c5ccccc5)c5c6ccccc6n(-c6ccccc6)c5n4)cc3)c2)cc1 ; c1ccc(-c2ccc(-c3cccc(-c4nc(-c5ccccc5)c5c6ccccc6n(-c6ccccc6)c5n4)c3)cc2)cc1]

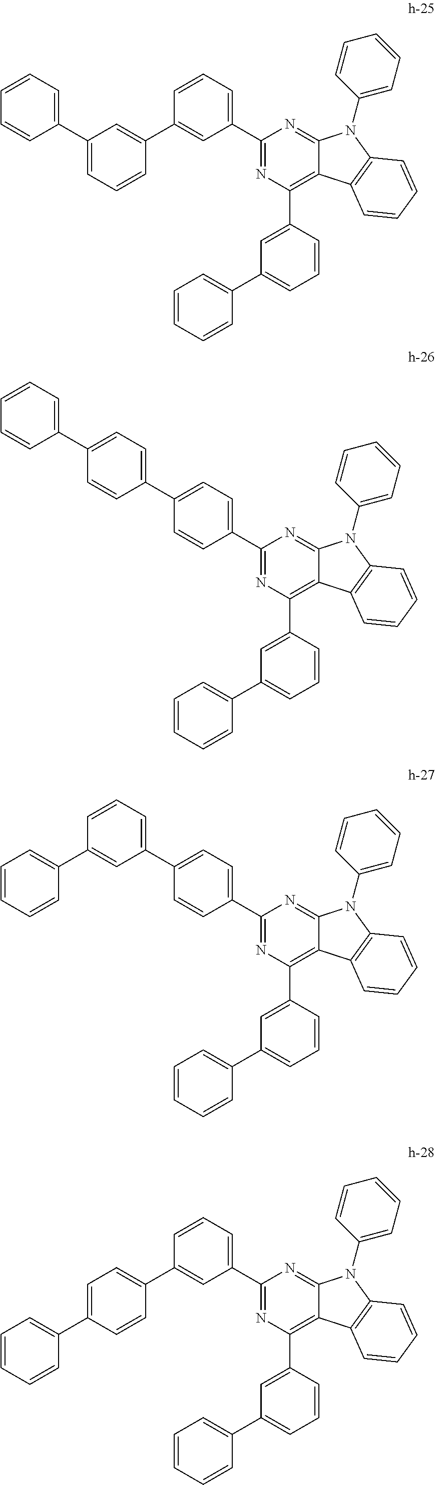 [SMILES: c1ccc(-c2cccc(-c3cccc(-c4nc(-c5cccc(-c6ccccc6)c5)c5c6ccccc6n(-c6ccccc6)c5n4)c3)c2)cc1 ; c1ccc(-c2ccc(-c3ccc(-c4nc(-c5cccc(-c6ccccc6)c5)c5c6ccccc6n(-c6ccccc6)c5n4)cc3)cc2)cc1 ; c1ccc(-c2cccc(-c3ccc(-c4nc(-c5cccc(-c6ccccc6)c5)c5c6ccccc6n(-c6ccccc6)c5n4)cc3)c2)cc1 ; c1ccc(-c2ccc(-c3cccc(-c4nc(-c5cccc(-c6ccccc6)c5)c5c6ccccc6n(-c6ccccc6)c5n4)c3)cc2)cc1]

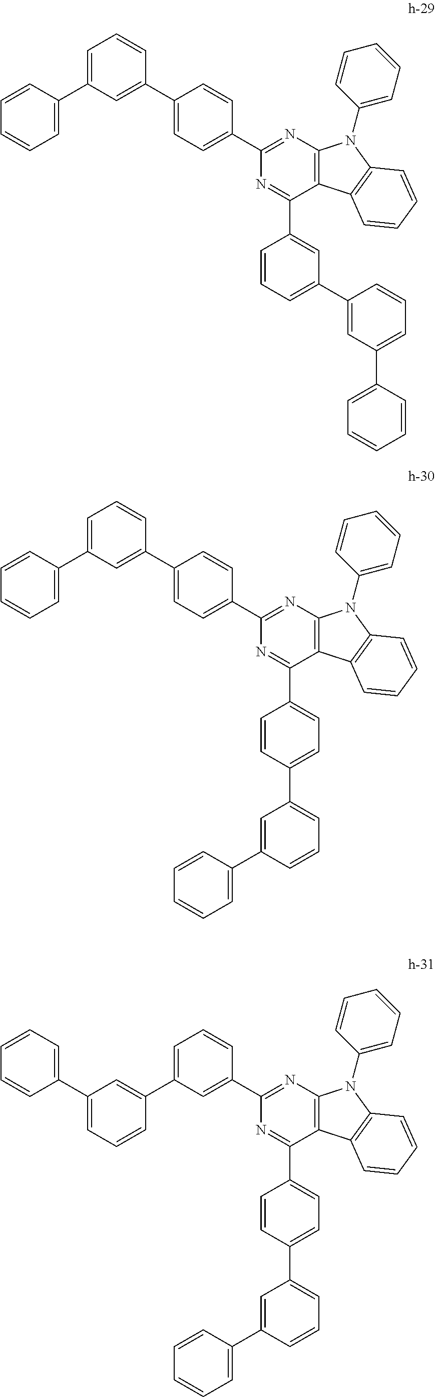 [SMILES: c1ccc(-c2cccc(-c3ccc(-c4nc(-c5cccc(-c6cccc(-c7ccccc7)c6)c5)c5c6ccccc6n(-c6ccccc6)c5n4)cc3)c2)cc1 ; c1ccc(-c2cccc(-c3ccc(-c4nc(-c5ccc(-c6cccc(-c7ccccc7)c6)cc5)c5c6ccccc6n(-c6ccccc6)c5n4)cc3)c2)cc1 ; c1ccc(-c2cccc(-c3ccc(-c4nc(-c5cccc(-c6cccc(-c7ccccc7)c6)c5)nc5c4c4ccccc4n5-c4ccccc4)cc3)c2)cc1]

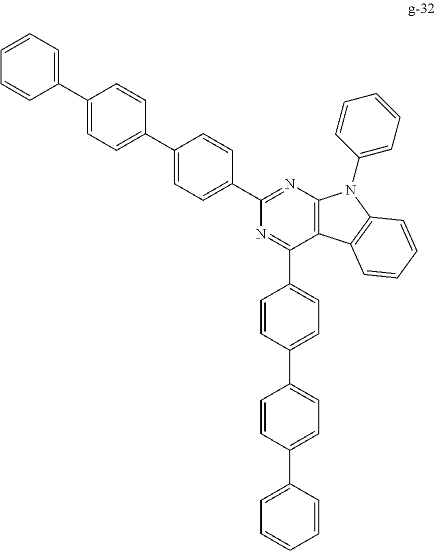 [SMILES: c1ccc(-c2ccc(-c3ccc(-c4nc(-c5ccc(-c6ccc(-c7ccccc7)cc6)cc5)c5c6ccccc6n(-c6ccccc6)c5n4)cc3)cc2)cc1]

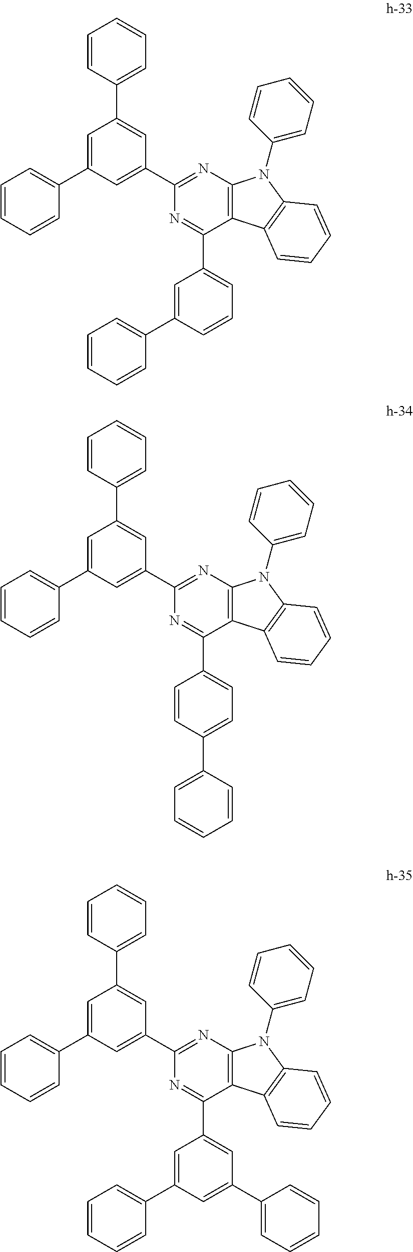

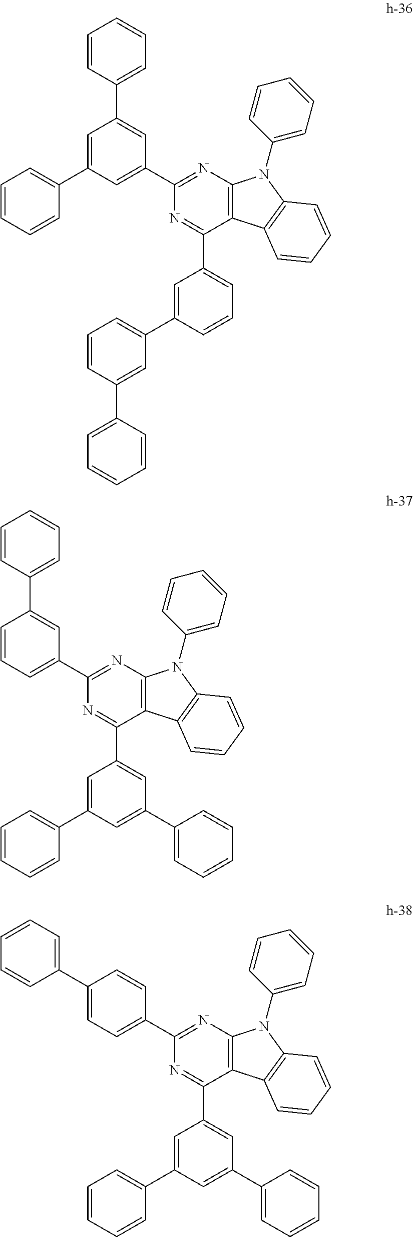

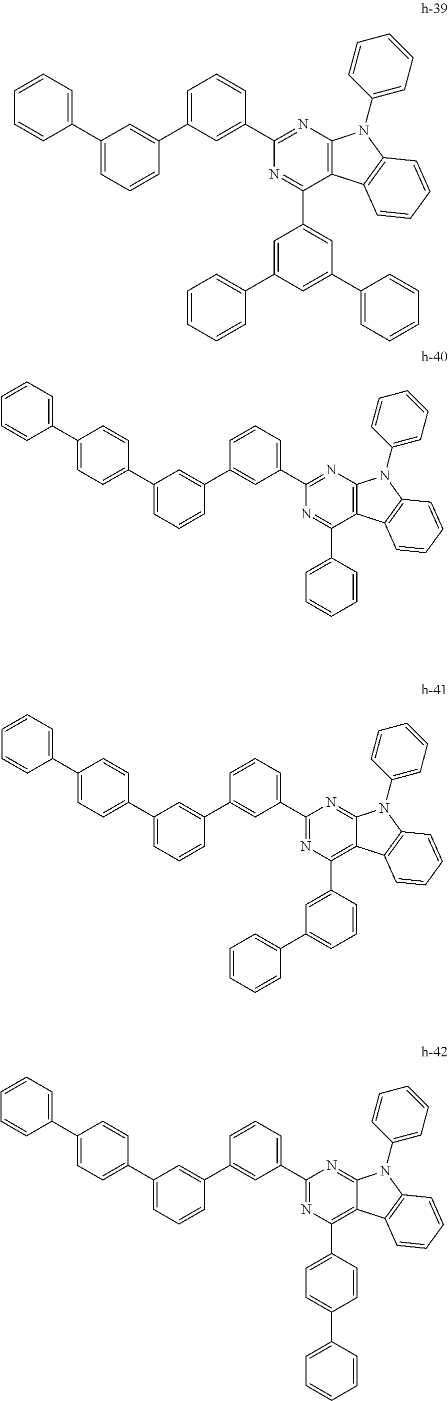 [SMILES: c1ccc(-c2cccc(-c3cccc(-c4nc(-c5cc(-c6ccccc6)cc(-c6ccccc6)c5)c5c6ccccc6n(-c6ccccc6)c5n4)c3)c2)cc1 ; c1ccc(-c2ccc(-c3cccc(-c4cccc(-c5nc(-c6ccccc6)c6c7ccccc7n(-c7ccccc7)c6n5)c4)c3)cc2)cc1 ; c1ccc(-c2ccc(-c3cccc(-c4cccc(-c5nc(-c6cccc(-c7ccccc7)c6)c6c7ccccc7n(-c7ccccc7)c6n5)c4)c3)cc2)cc1 ; c1ccc(-c2ccc(-c3cccc(-c4cccc(-c5nc(-c6ccc(-c7ccccc7)cc6)c6c7ccccc7n(-c7ccccc7)c6n5)c4)c3)cc2)cc1]

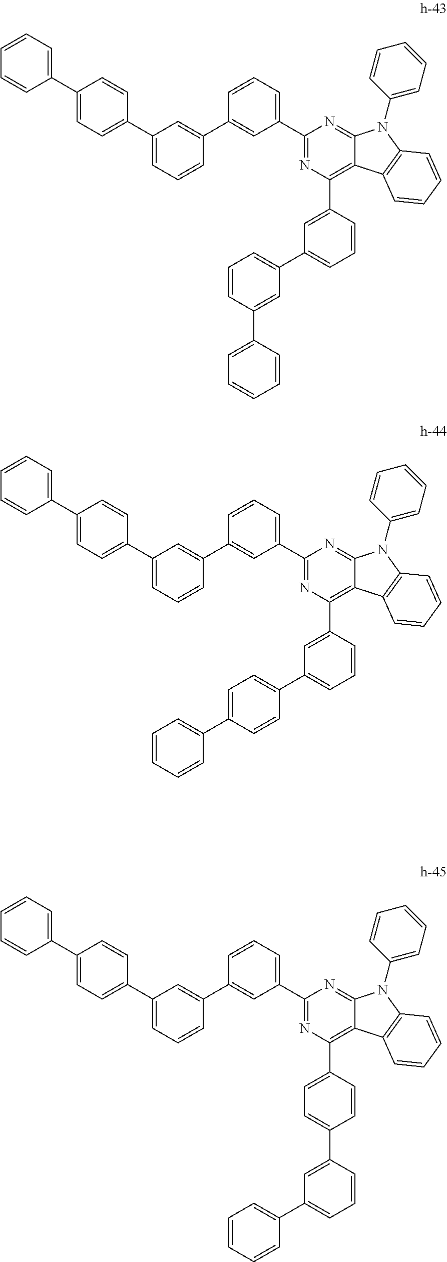 [SMILES: c1ccc(-c2ccc(-c3cccc(-c4cccc(-c5nc(-c6cccc(-c7cccc(-c8ccccc8)c7)c6)c6c7ccccc7n(-c7ccccc7)c6n5)c4)c3)cc2)cc1 ; c1ccc(-c2ccc(-c3cccc(-c4cccc(-c5nc(-c6cccc(-c7ccc(-c8ccccc8)cc7)c6)c6c7ccccc7n(-c7ccccc7)c6n5)c4)c3)cc2)cc1 ; c1ccc(-c2ccc(-c3cccc(-c4cccc(-c5nc(-c6ccc(-c7cccc(-c8ccccc8)c7)cc6)c6c7ccccc7n(-c7ccccc7)c6n5)c4)c3)cc2)cc1]

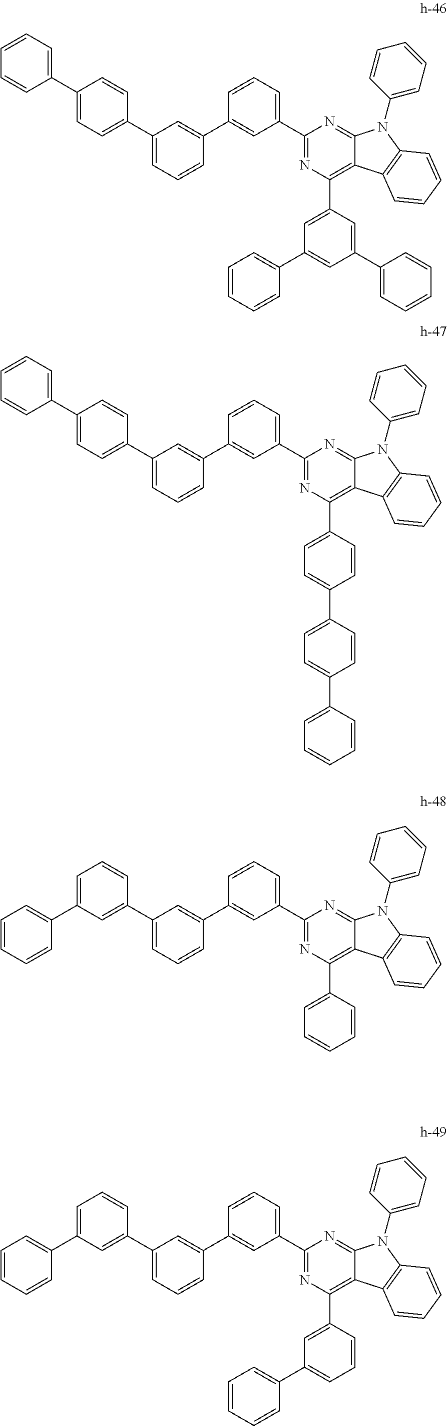 [SMILES: c1ccc(-c2ccc(-c3cccc(-c4cccc(-c5nc(-c6cc(-c7ccccc7)cc(-c7ccccc7)c6)c6c7ccccc7n(-c7ccccc7)c6n5)c4)c3)cc2)cc1 ; c1ccc(-c2ccc(-c3ccc(-c4nc(-c5cccc(-c6cccc(-c7ccc(-c8ccccc8)cc7)c6)c5)nc5c4c4ccccc4n5-c4ccccc4)cc3)cc2)cc1 ; c1ccc(-c2cccc(-c3cccc(-c4cccc(-c5nc(-c6ccccc6)c6c7ccccc7n(-c7ccccc7)c6n5)c4)c3)c2)cc1 ; c1ccc(-c2cccc(-c3cccc(-c4cccc(-c5nc(-c6cccc(-c7ccccc7)c6)c6c7ccccc7n(-c7ccccc7)c6n5)c4)c3)c2)cc1]

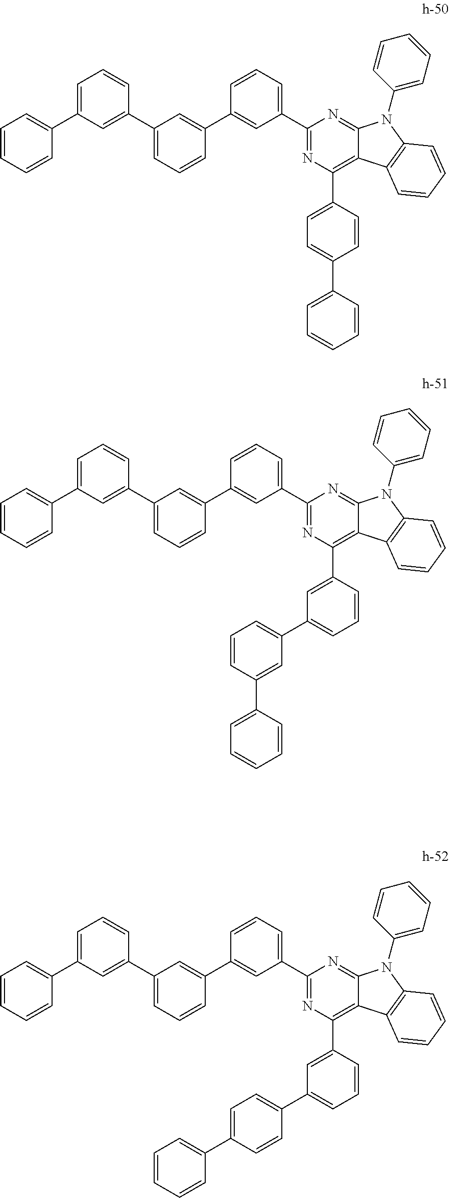 [SMILES: c1ccc(-c2ccc(-c3nc(-c4cccc(-c5cccc(-c6cccc(-c7ccccc7)c6)c5)c4)nc4c3c3ccccc3n4-c3ccccc3)cc2)cc1 ; c1ccc(-c2cccc(-c3cccc(-c4cccc(-c5nc(-c6cccc(-c7cccc(-c8ccccc8)c7)c6)c6c7ccccc7n(-c7ccccc7)c6n5)c4)c3)c2)cc1 ; c1ccc(-c2ccc(-c3cccc(-c4nc(-c5cccc(-c6cccc(-c7cccc(-c8ccccc8)c7)c6)c5)nc5c4c4ccccc4n5-c4ccccc4)c3)cc2)cc1]

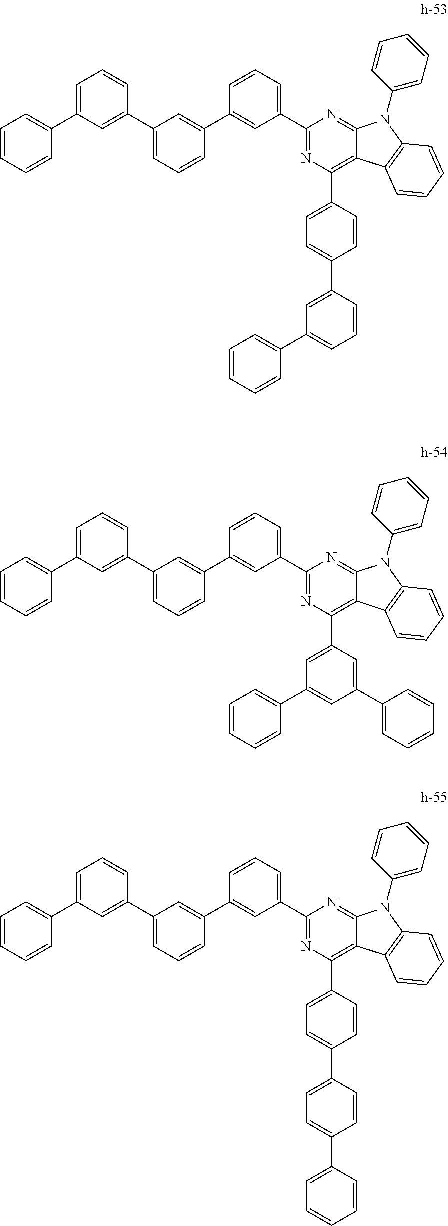 [SMILES: c1ccc(-c2cccc(-c3ccc(-c4nc(-c5cccc(-c6cccc(-c7cccc(-c8ccccc8)c7)c6)c5)nc5c4c4ccccc4n5-c4ccccc4)cc3)c2)cc1 ; c1ccc(-c2cccc(-c3cccc(-c4cccc(-c5nc(-c6cc(-c7ccccc7)cc(-c7ccccc7)c6)c6c7ccccc7n(-c7ccccc7)c6n5)c4)c3)c2)cc1 ; c1ccc(-c2ccc(-c3ccc(-c4nc(-c5cccc(-c6cccc(-c7cccc(-c8ccccc8)c7)c6)c5)nc5c4c4ccccc4n5-c4ccccc4)cc3)cc2)cc1]

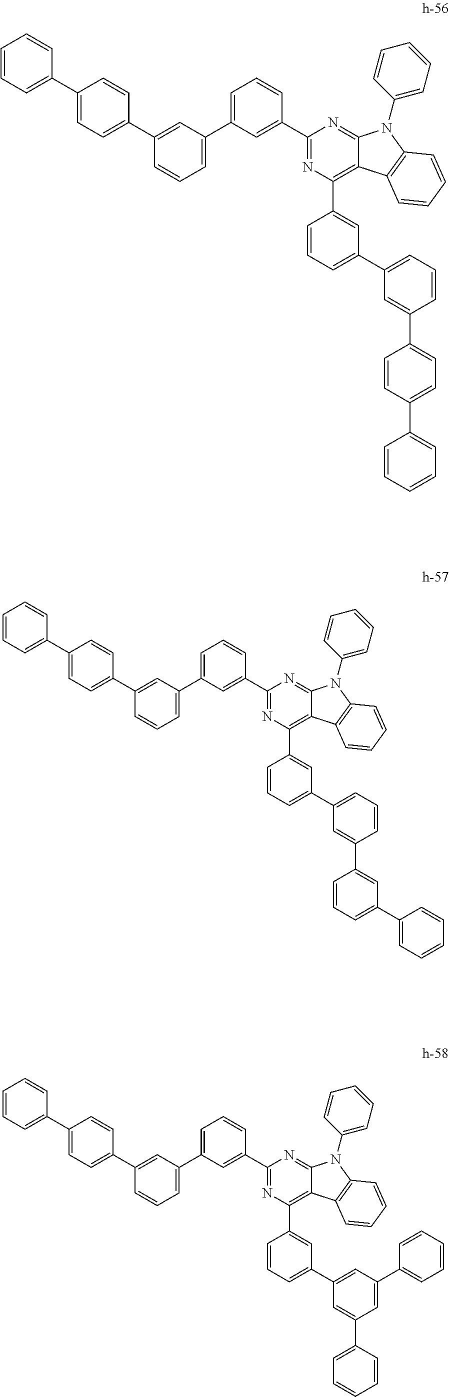 [SMILES: c1ccc(-c2ccc(-c3cccc(-c4cccc(-c5nc(-c6cccc(-c7cccc(-c8ccc(-c9ccccc9)cc8)c7)c6)c6c7ccccc7n(-c7ccccc7)c6n5)c4)c3)cc2)cc1 ; c1ccc(-c2ccc(-c3cccc(-c4cccc(-c5nc(-c6cccc(-c7cccc(-c8cccc(-c9ccccc9)c8)c7)c6)c6c7ccccc7n(-c7ccccc7)c6n5)c4)c3)cc2)cc1 ; c1ccc(-c2ccc(-c3cccc(-c4cccc(-c5nc(-c6cccc(-c7cc(-c8ccccc8)cc(-c8ccccc8)c7)c6)c6c7ccccc7n(-c7ccccc7)c6n5)c4)c3)cc2)cc1]

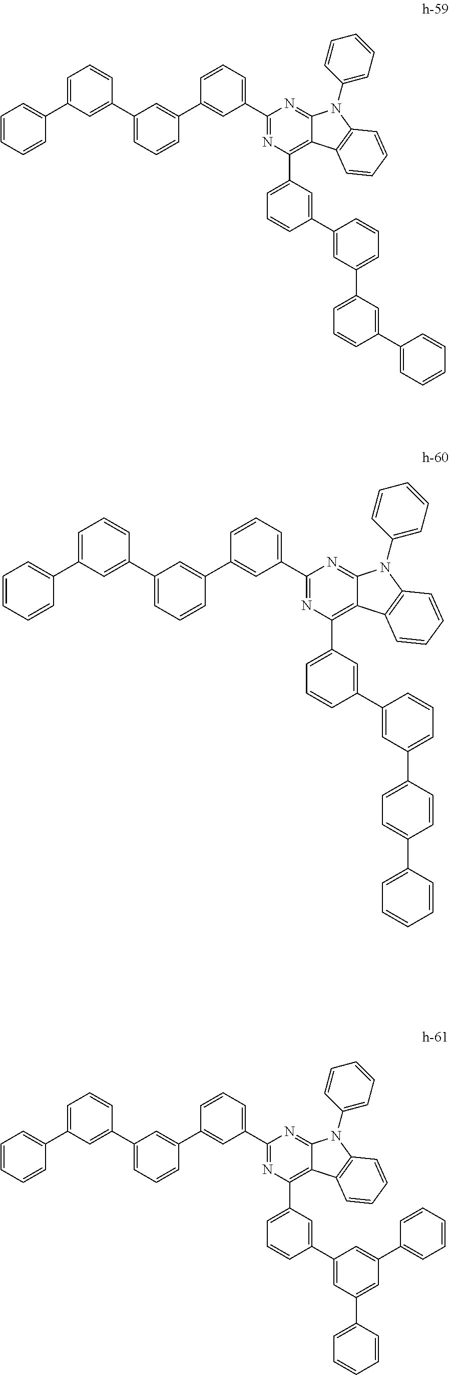 [SMILES: c1ccc(-c2cccc(-c3cccc(-c4cccc(-c5nc(-c6cccc(-c7cccc(-c8cccc(-c9ccccc9)c8)c7)c6)c6c7ccccc7n(-c7ccccc7)c6n5)c4)c3)c2)cc1 ; c1ccc(-c2ccc(-c3cccc(-c4cccc(-c5nc(-c6cccc(-c7cccc(-c8cccc(-c9ccccc9)c8)c7)c6)nc6c5c5ccccc5n6-c5ccccc5)c4)c3)cc2)cc1 ; c1ccc(-c2cccc(-c3cccc(-c4cccc(-c5nc(-c6cccc(-c7cc(-c8ccccc8)cc(-c8ccccc8)c7)c6)c6c7ccccc7n(-c7ccccc7)c6n5)c4)c3)c2)cc1]

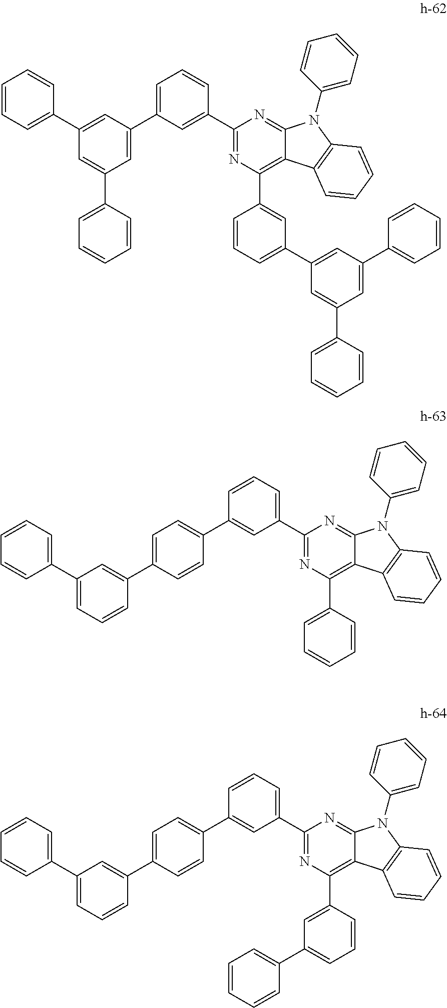 [SMILES: c1ccc(-c2cc(-c3ccccc3)cc(-c3cccc(-c4nc(-c5cccc(-c6cc(-c7ccccc7)cc(-c7ccccc7)c6)c5)c5c6ccccc6n(-c6ccccc6)c5n4)c3)c2)cc1 ; c1ccc(-c2cccc(-c3ccc(-c4cccc(-c5nc(-c6ccccc6)c6c7ccccc7n(-c7ccccc7)c6n5)c4)cc3)c2)cc1 ; c1ccc(-c2cccc(-c3ccc(-c4cccc(-c5nc(-c6cccc(-c7ccccc7)c6)c6c7ccccc7n(-c7ccccc7)c6n5)c4)cc3)c2)cc1]

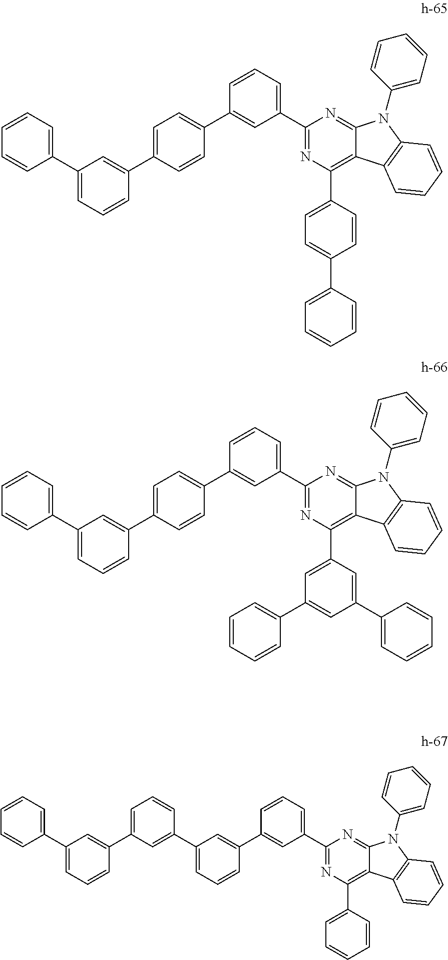 [SMILES: c1ccc(-c2ccc(-c3nc(-c4cccc(-c5ccc(-c6cccc(-c7ccccc7)c6)cc5)c4)nc4c3c3ccccc3n4-c3ccccc3)cc2)cc1 ; c1ccc(-c2cccc(-c3ccc(-c4cccc(-c5nc(-c6cc(-c7ccccc7)cc(-c7ccccc7)c6)c6c7ccccc7n(-c7ccccc7)c6n5)c4)cc3)c2)cc1 ; c1ccc(-c2cccc(-c3cccc(-c4cccc(-c5cccc(-c6nc(-c7ccccc7)c7c8ccccc8n(-c8ccccc8)c7n6)c5)c4)c3)c2)cc1]

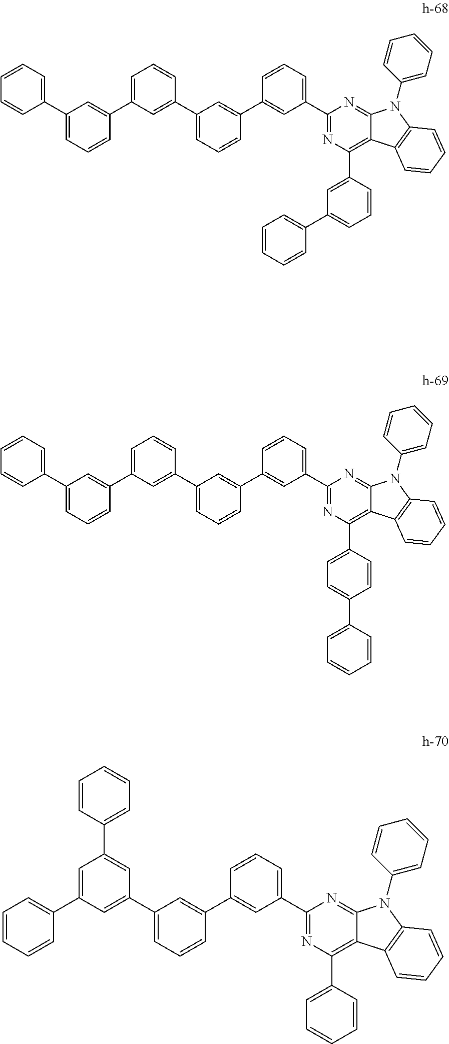 [SMILES: c1ccc(-c2cccc(-c3cccc(-c4cccc(-c5cccc(-c6nc(-c7cccc(-c8ccccc8)c7)c7c8ccccc8n(-c8ccccc8)c7n6)c5)c4)c3)c2)cc1 ; c1ccc(-c2ccc(-c3nc(-c4cccc(-c5cccc(-c6cccc(-c7cccc(-c8ccccc8)c7)c6)c5)c4)nc4c3c3ccccc3n4-c3ccccc3)cc2)cc1 ; c1ccc(-c2cc(-c3ccccc3)cc(-c3cccc(-c4cccc(-c5nc(-c6ccccc6)c6c7ccccc7n(-c7ccccc7)c6n5)c4)c3)c2)cc1]

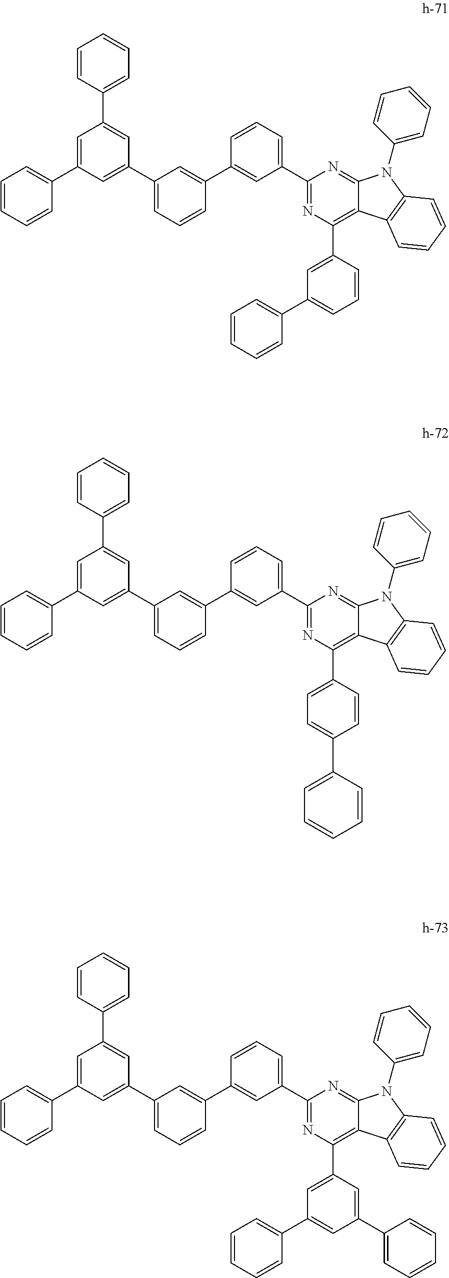 [SMILES: c1ccc(-c2cc(-c3ccccc3)cc(-c3cccc(-c4cccc(-c5nc(-c6cccc(-c7ccccc7)c6)c6c7ccccc7n(-c7ccccc7)c6n5)c4)c3)c2)cc1 ; c1ccc(-c2ccc(-c3nc(-c4cccc(-c5cccc(-c6cc(-c7ccccc7)cc(-c7ccccc7)c6)c5)c4)nc4c3c3ccccc3n4-c3ccccc3)cc2)cc1 ; c1ccc(-c2cc(-c3ccccc3)cc(-c3cccc(-c4cccc(-c5nc(-c6cc(-c7ccccc7)cc(-c7ccccc7)c6)c6c7ccccc7n(-c7ccccc7)c6n5)c4)c3)c2)cc1]

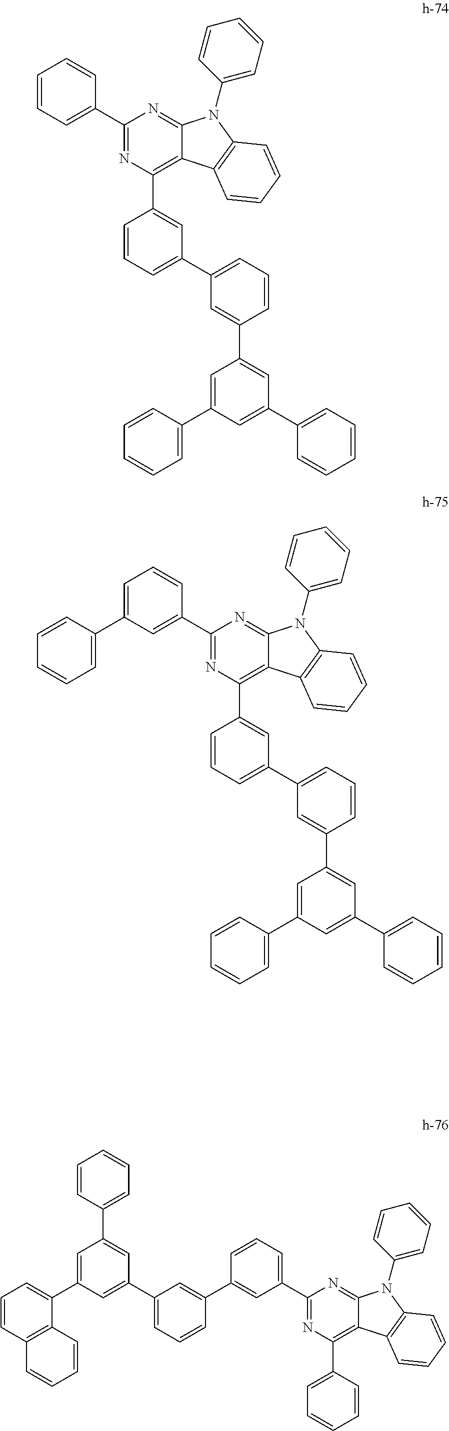 [SMILES: c1ccc(-c2cc(-c3ccccc3)cc(-c3cccc(-c4cccc(-c5nc(-c6ccccc6)nc6c5c5ccccc5n6-c5ccccc5)c4)c3)c2)cc1 ; c1ccc(-c2cc(-c3ccccc3)cc(-c3cccc(-c4cccc(-c5nc(-c6cccc(-c7ccccc7)c6)nc6c5c5ccccc5n6-c5ccccc5)c4)c3)c2)cc1 ; c1ccc(-c2cc(-c3cccc(-c4cccc(-c5nc(-c6ccccc6)c6c7ccccc7n(-c7ccccc7)c6n5)c4)c3)cc(-c3cccc4ccccc34)c2)cc1]

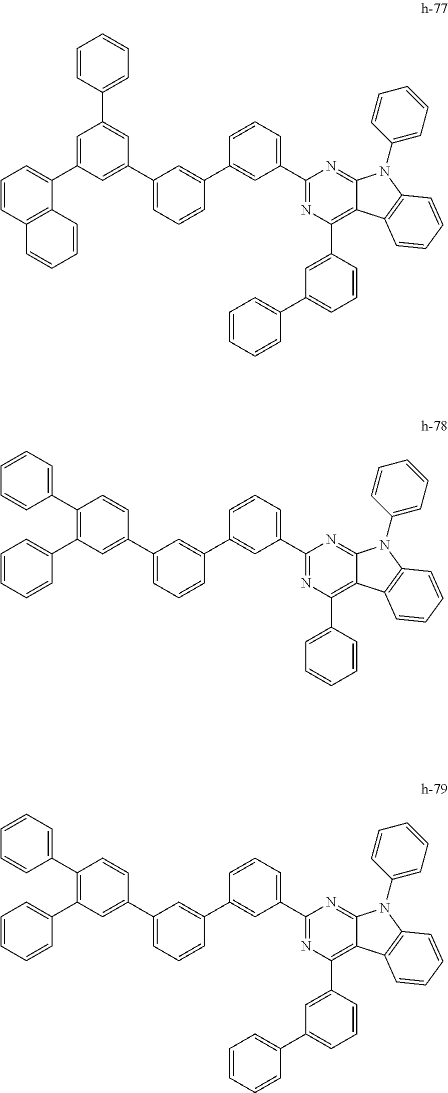 [SMILES: c1ccc(-c2cc(-c3cccc(-c4cccc(-c5nc(-c6cccc(-c7ccccc7)c6)c6c7ccccc7n(-c7ccccc7)c6n5)c4)c3)cc(-c3cccc4ccccc34)c2)cc1 ; c1ccc(-c2ccc(-c3cccc(-c4cccc(-c5nc(-c6ccccc6)c6c7ccccc7n(-c7ccccc7)c6n5)c4)c3)cc2-c2ccccc2)cc1 ; c1ccc(-c2cccc(-c3nc(-c4cccc(-c5cccc(-c6ccc(-c7ccccc7)c(-c7ccccc7)c6)c5)c4)nc4c3c3ccccc3n4-c3ccccc3)c2)cc1]

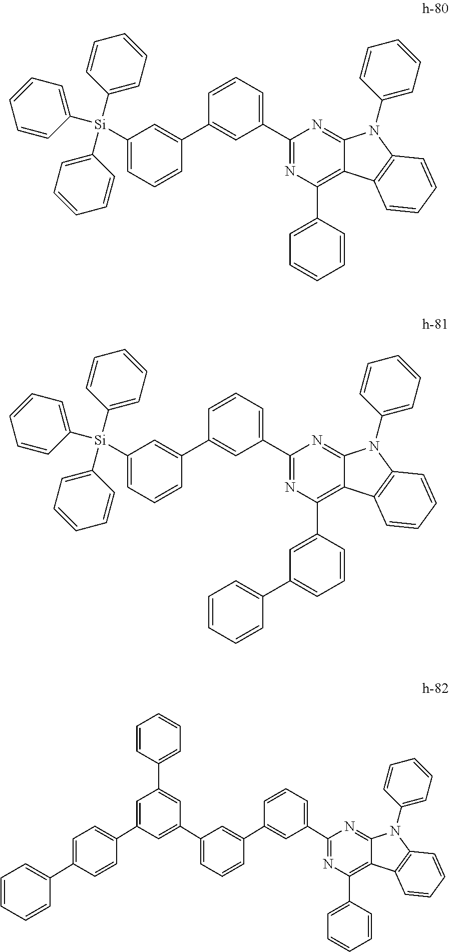 [SMILES: c1ccc(-c2nc(-c3cccc(-c4cccc([Si](c5ccccc5)(c5ccccc5)c5ccccc5)c4)c3)nc3c2c2ccccc2n3-c2ccccc2)cc1 ; c1ccc(-c2cccc(-c3nc(-c4cccc(-c5cccc([Si](c6ccccc6)(c6ccccc6)c6ccccc6)c5)c4)nc4c3c3ccccc3n4-c3ccccc3)c2)cc1 ; c1ccc(-c2ccc(-c3cc(-c4ccccc4)cc(-c4cccc(-c5cccc(-c6nc(-c7ccccc7)c7c8ccccc8n(-c8ccccc8)c7n6)c5)c4)c3)cc2)cc1]

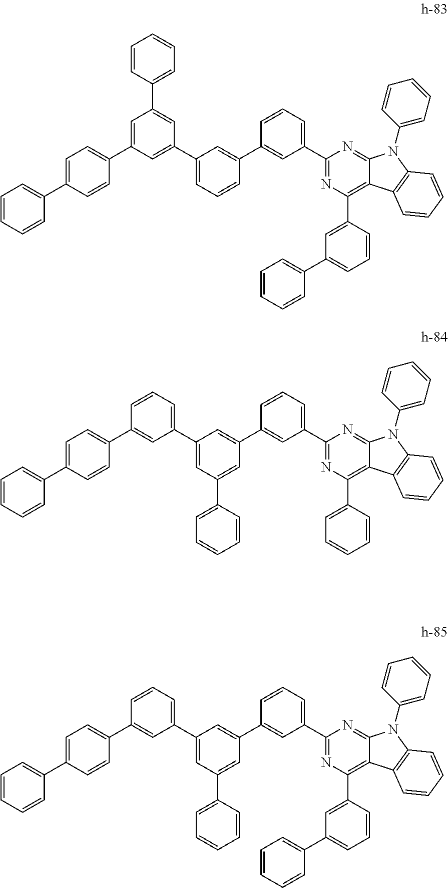 [SMILES: c1ccc(-c2ccc(-c3cc(-c4ccccc4)cc(-c4cccc(-c5cccc(-c6nc(-c7cccc(-c8ccccc8)c7)c7c8ccccc8n(-c8ccccc8)c7n6)c5)c4)c3)cc2)cc1 ; c1ccc(-c2ccc(-c3cccc(-c4cc(-c5ccccc5)cc(-c5cccc(-c6nc(-c7ccccc7)c7c8ccccc8n(-c8ccccc8)c7n6)c5)c4)c3)cc2)cc1 ; c1ccc(-c2ccc(-c3cccc(-c4cc(-c5ccccc5)cc(-c5cccc(-c6nc(-c7cccc(-c8ccccc8)c7)c7c8ccccc8n(-c8ccccc8)c7n6)c5)c4)c3)cc2)cc1]

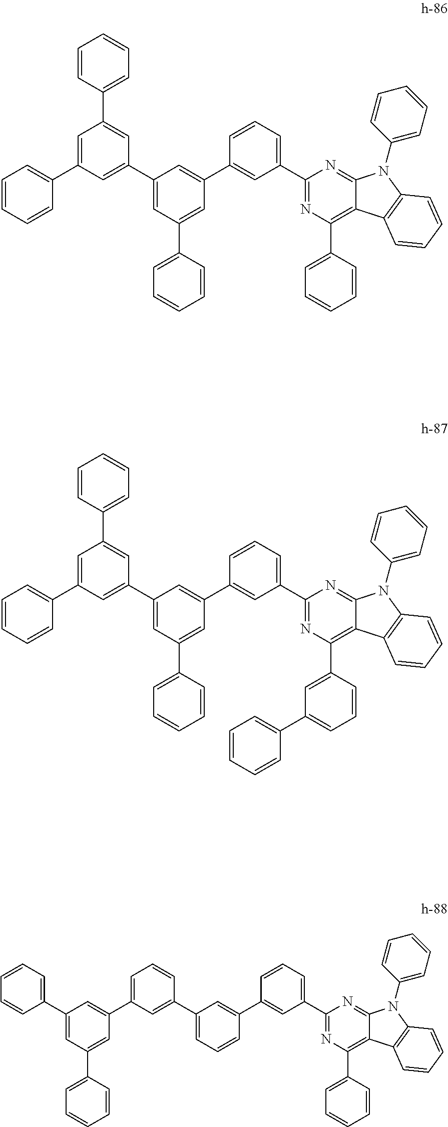 [SMILES: c1ccc(-c2cc(-c3ccccc3)cc(-c3cc(-c4ccccc4)cc(-c4cccc(-c5nc(-c6ccccc6)c6c7ccccc7n(-c7ccccc7)c6n5)c4)c3)c2)cc1 ; c1ccc(-c2cc(-c3ccccc3)cc(-c3cc(-c4ccccc4)cc(-c4cccc(-c5nc(-c6cccc(-c7ccccc7)c6)c6c7ccccc7n(-c7ccccc7)c6n5)c4)c3)c2)cc1 ; c1ccc(-c2cc(-c3ccccc3)cc(-c3cccc(-c4cccc(-c5cccc(-c6nc(-c7ccccc7)c7c8ccccc8n(-c8ccccc8)c7n6)c5)c4)c3)c2)cc1]

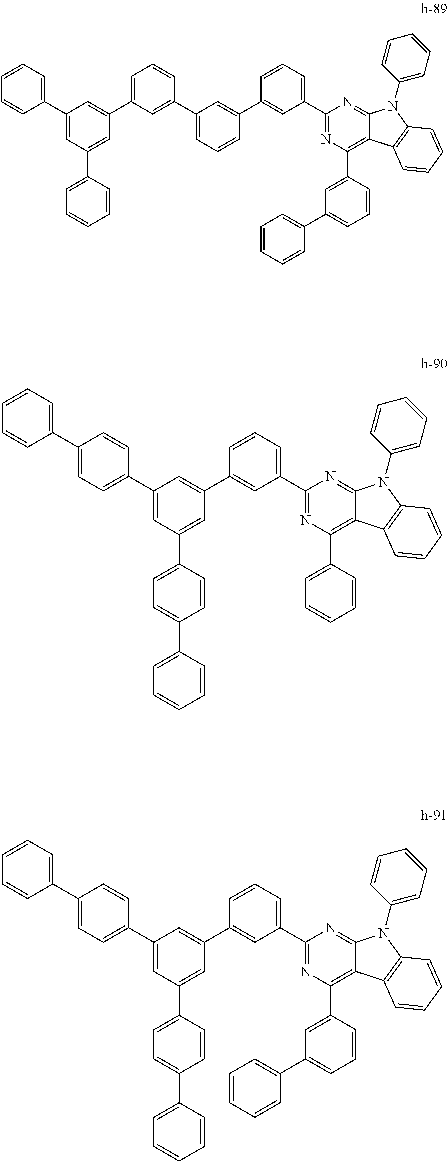 [SMILES: c1ccc(-c2cc(-c3ccccc3)cc(-c3cccc(-c4cccc(-c5cccc(-c6nc(-c7cccc(-c8ccccc8)c7)c7c8ccccc8n(-c8ccccc8)c7n6)c5)c4)c3)c2)cc1 ; c1ccc(-c2ccc(-c3cc(-c4ccc(-c5ccccc5)cc4)cc(-c4cccc(-c5nc(-c6ccccc6)c6c7ccccc7n(-c7ccccc7)c6n5)c4)c3)cc2)cc1 ; c1ccc(-c2ccc(-c3cc(-c4ccc(-c5ccccc5)cc4)cc(-c4cccc(-c5nc(-c6cccc(-c7ccccc7)c6)c6c7ccccc7n(-c7ccccc7)c6n5)c4)c3)cc2)cc1]

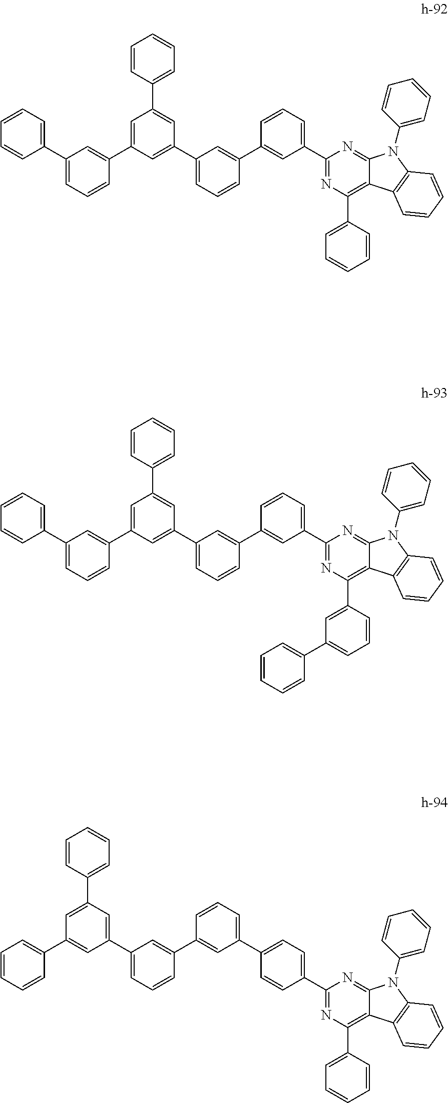 [SMILES: c1ccc(-c2cccc(-c3cc(-c4ccccc4)cc(-c4cccc(-c5cccc(-c6nc(-c7ccccc7)c7c8ccccc8n(-c8ccccc8)c7n6)c5)c4)c3)c2)cc1 ; c1ccc(-c2cccc(-c3cc(-c4ccccc4)cc(-c4cccc(-c5cccc(-c6nc(-c7cccc(-c8ccccc8)c7)c7c8ccccc8n(-c8ccccc8)c7n6)c5)c4)c3)c2)cc1 ; c1ccc(-c2cc(-c3ccccc3)cc(-c3cccc(-c4cccc(-c5ccc(-c6nc(-c7ccccc7)c7c8ccccc8n(-c8ccccc8)c7n6)cc5)c4)c3)c2)cc1]

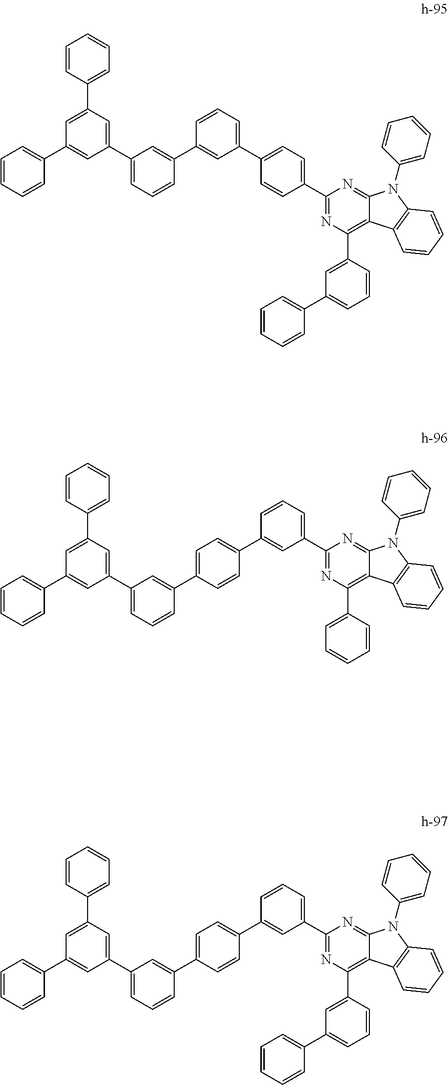 [SMILES: c1ccc(-c2cc(-c3ccccc3)cc(-c3cccc(-c4cccc(-c5ccc(-c6nc(-c7cccc(-c8ccccc8)c7)c7c8ccccc8n(-c8ccccc8)c7n6)cc5)c4)c3)c2)cc1 ; c1ccc(-c2cc(-c3ccccc3)cc(-c3cccc(-c4ccc(-c5cccc(-c6nc(-c7ccccc7)c7c8ccccc8n(-c8ccccc8)c7n6)c5)cc4)c3)c2)cc1 ; c1ccc(-c2cc(-c3ccccc3)cc(-c3cccc(-c4ccc(-c5cccc(-c6nc(-c7cccc(-c8ccccc8)c7)c7c8ccccc8n(-c8ccccc8)c7n6)c5)cc4)c3)c2)cc1]

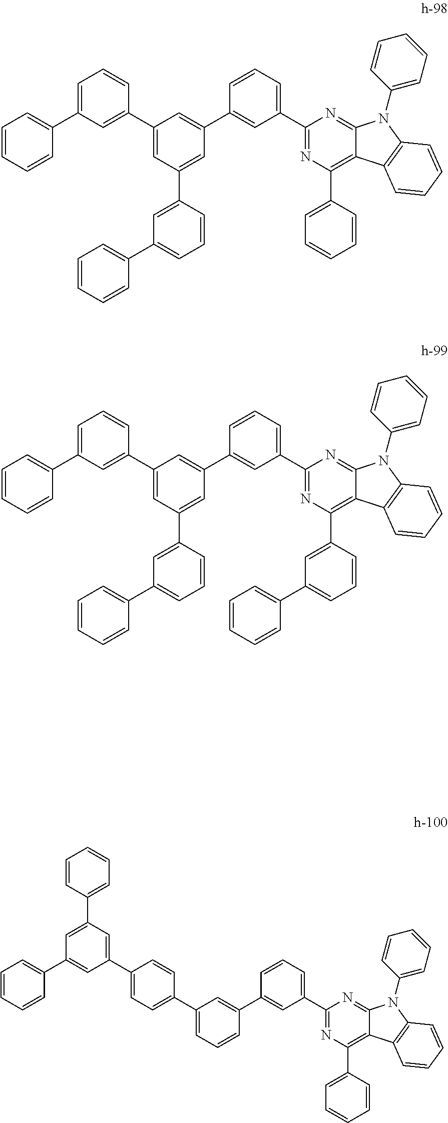 [SMILES: c1ccc(-c2cccc(-c3cc(-c4cccc(-c5ccccc5)c4)cc(-c4cccc(-c5nc(-c6ccccc6)c6c7ccccc7n(-c7ccccc7)c6n5)c4)c3)c2)cc1 ; c1ccc(-c2cccc(-c3cc(-c4cccc(-c5ccccc5)c4)cc(-c4cccc(-c5nc(-c6cccc(-c7ccccc7)c6)c6c7ccccc7n(-c7ccccc7)c6n5)c4)c3)c2)cc1 ; c1ccc(-c2cc(-c3ccccc3)cc(-c3ccc(-c4cccc(-c5cccc(-c6nc(-c7ccccc7)c7c8ccccc8n(-c8ccccc8)c7n6)c5)c4)cc3)c2)cc1]

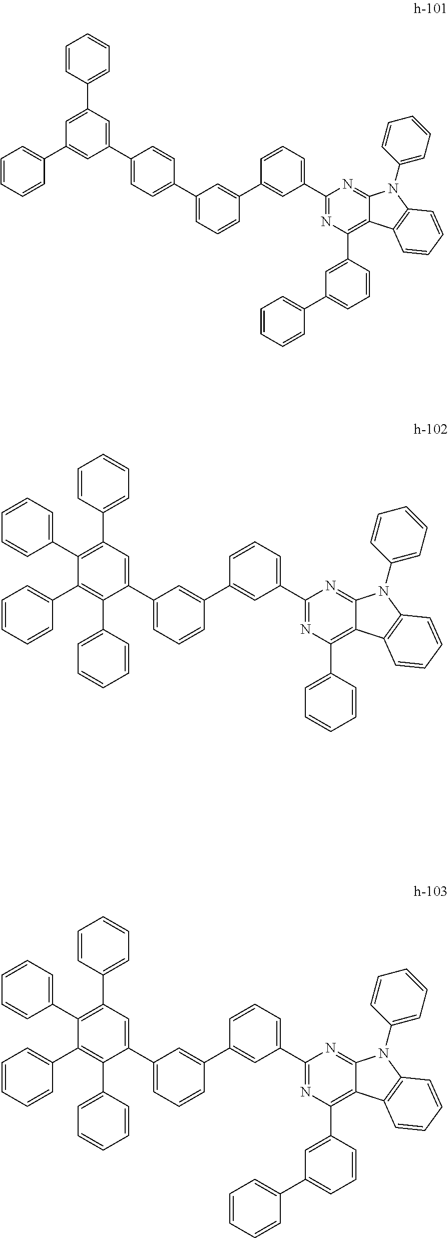 [SMILES: c1ccc(-c2cc(-c3ccccc3)cc(-c3ccc(-c4cccc(-c5cccc(-c6nc(-c7cccc(-c8ccccc8)c7)c7c8ccccc8n(-c8ccccc8)c7n6)c5)c4)cc3)c2)cc1 ; c1ccc(-c2cc(-c3cccc(-c4cccc(-c5nc(-c6ccccc6)c6c7ccccc7n(-c7ccccc7)c6n5)c4)c3)c(-c3ccccc3)c(-c3ccccc3)c2-c2ccccc2)cc1 ; c1ccc(-c2cccc(-c3nc(-c4cccc(-c5cccc(-c6cc(-c7ccccc7)c(-c7ccccc7)c(-c7ccccc7)c6-c6ccccc6)c5)c4)nc4c3c3ccccc3n4-c3ccccc3)c2)cc1]

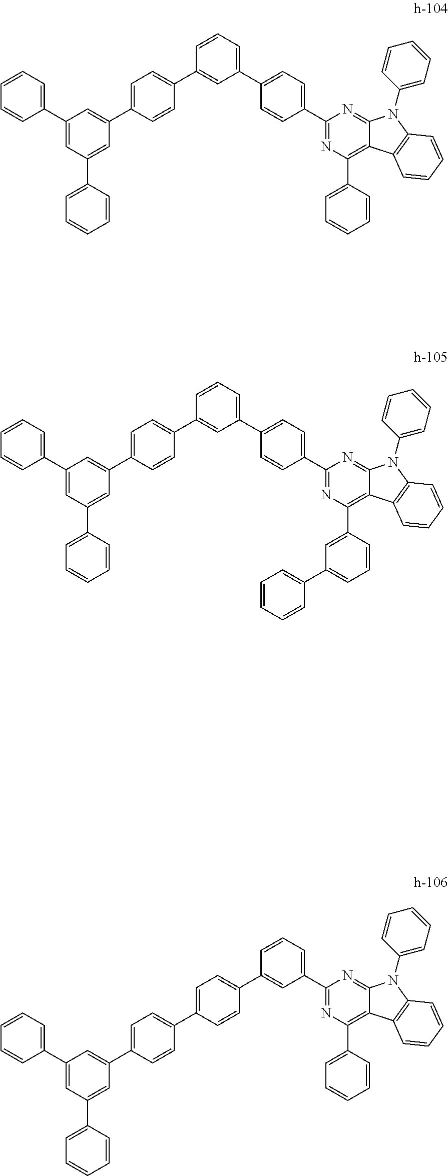 [SMILES: c1ccc(-c2cc(-c3ccccc3)cc(-c3ccc(-c4cccc(-c5ccc(-c6nc(-c7ccccc7)c7c8ccccc8n(-c8ccccc8)c7n6)cc5)c4)cc3)c2)cc1 ; c1ccc(-c2cc(-c3ccccc3)cc(-c3ccc(-c4cccc(-c5ccc(-c6nc(-c7cccc(-c8ccccc8)c7)c7c8ccccc8n(-c8ccccc8)c7n6)cc5)c4)cc3)c2)cc1 ; c1ccc(-c2cc(-c3ccccc3)cc(-c3ccc(-c4ccc(-c5cccc(-c6nc(-c7ccccc7)c7c8ccccc8n(-c8ccccc8)c7n6)c5)cc4)cc3)c2)cc1]

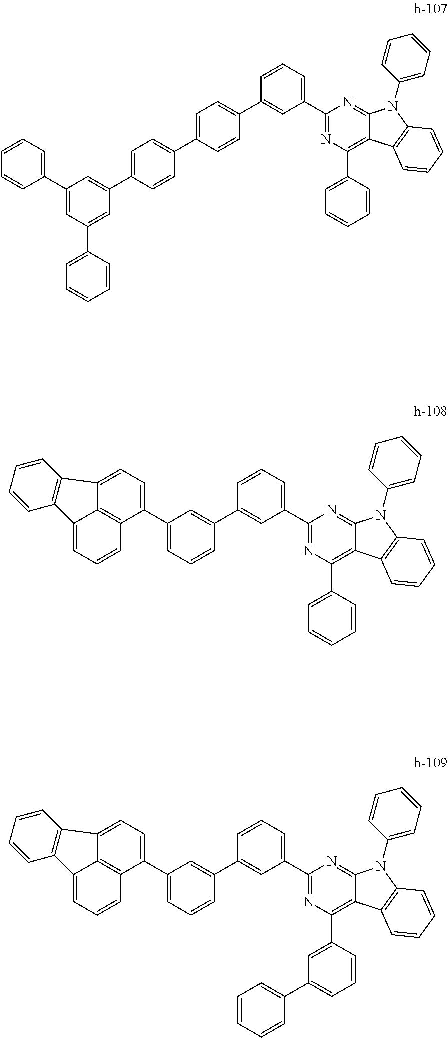 [SMILES: c1ccc(-c2cc(-c3ccccc3)cc(-c3ccc(-c4ccc(-c5cccc(-c6nc(-c7ccccc7)c7c8ccccc8n(-c8ccccc8)c7n6)c5)cc4)cc3)c2)cc1 ; c1ccc(-c2nc(-c3cccc(-c4cccc(-c5ccc6c7c(cccc57)-c5ccccc5-6)c4)c3)nc3c2c2ccccc2n3-c2ccccc2)cc1 ; c1ccc(-c2cccc(-c3nc(-c4cccc(-c5cccc(-c6ccc7c8c(cccc68)-c6ccccc6-7)c5)c4)nc4c3c3ccccc3n4-c3ccccc3)c2)cc1]

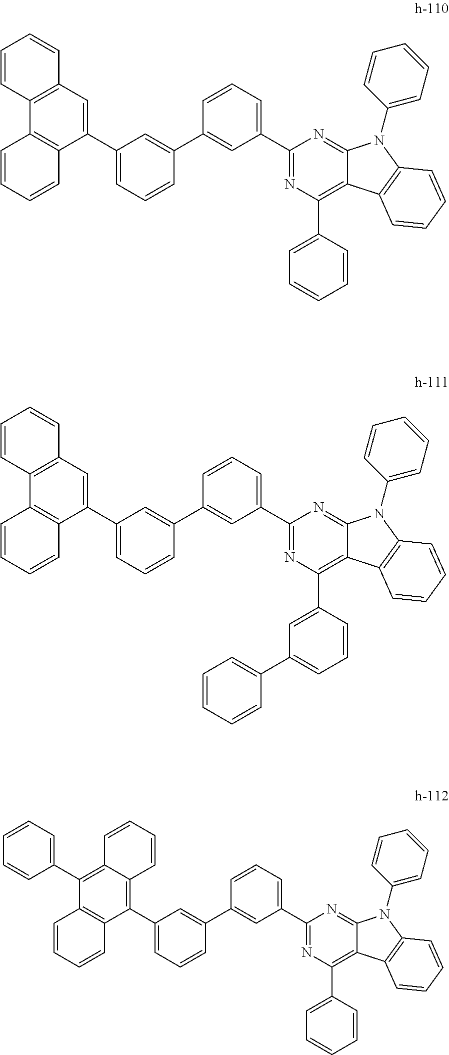 [SMILES: c1ccc(-c2nc(-c3cccc(-c4cccc(-c5cc6ccccc6c6ccccc56)c4)c3)nc3c2c2ccccc2n3-c2ccccc2)cc1 ; c1ccc(-c2cccc(-c3nc(-c4cccc(-c5cccc(-c6cc7ccccc7c7ccccc67)c5)c4)nc4c3c3ccccc3n4-c3ccccc3)c2)cc1 ; c1ccc(-c2c3ccccc3c(-c3cccc(-c4cccc(-c5nc(-c6ccccc6)c6c7ccccc7n(-c7ccccc7)c6n5)c4)c3)c3ccccc23)cc1]

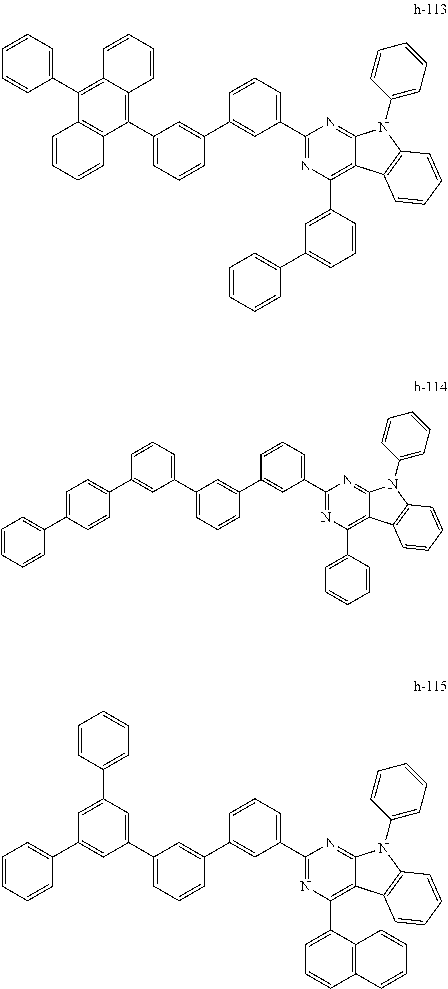 [SMILES: c1ccc(-c2cccc(-c3nc(-c4cccc(-c5cccc(-c6c7ccccc7c(-c7ccccc7)c7ccccc67)c5)c4)nc4c3c3ccccc3n4-c3ccccc3)c2)cc1 ; c1ccc(-c2ccc(-c3cccc(-c4cccc(-c5cccc(-c6nc(-c7ccccc7)c7c8ccccc8n(-c8ccccc8)c7n6)c5)c4)c3)cc2)cc1 ; c1ccc(-c2cc(-c3ccccc3)cc(-c3cccc(-c4cccc(-c5nc(-c6cccc7ccccc67)c6c7ccccc7n(-c7ccccc7)c6n5)c4)c3)c2)cc1]

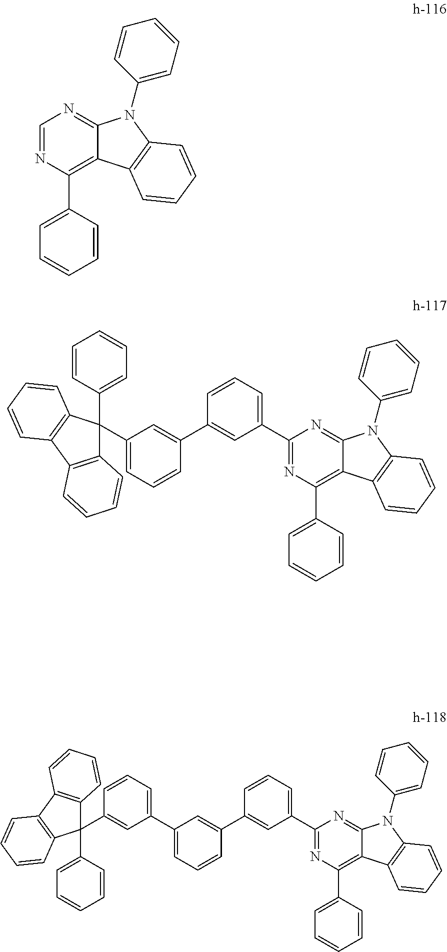 [SMILES: c1ccc(-c2ncnc3c2c2ccccc2n3-c2ccccc2)cc1 ; c1ccc(-c2nc(-c3cccc(-c4cccc(C5(c6ccccc6)c6ccccc6-c6ccccc65)c4)c3)nc3c2c2ccccc2n3-c2ccccc2)cc1 ; c1ccc(-c2nc(-c3cccc(-c4cccc(-c5cccc(C6(c7ccccc7)c7ccccc7-c7ccccc76)c5)c4)c3)nc3c2c2ccccc2n3-c2ccccc2)cc1]

In Formula 1 above, at least one of R₂ and R₃ may be selected from a substituted or unsubstituted C₆-C₆₀ aryl group, and a substituted or unsubstituted monovalent non-aromatic condensed polycyclic group. Thus, the condensed cyclic compound of Formula 1 above may have a highest occupied molecular orbital (HOMO) energy level, a lowest unoccupied molecular orbital (LUMO) energy level, a T1 energy level, and an S1 energy level that are appropriate for a material for an organic light emitting device, for example, a host material for the EML (for example, a host material for the EML including both a host and a dopant). The condensed cyclic compound of Formula 1 may have good thermal and electrical stabilities, and accordingly, an organic light-emitting device using the condensed cyclic compound of Formula 1 may have high efficiency and long lifetime characteristics.

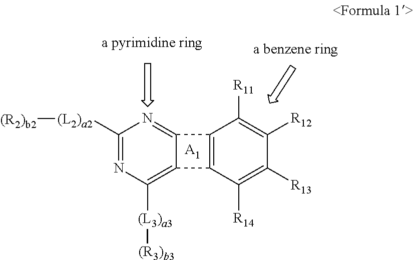

The condensed cyclic compound of Formula 1 above has a core in which a pyrimidine ring and a benzene ring are condensed to opposite sides of the ring A₁, respectively (refer to Formula 1′ above), and accordingly may have a HOMO energy level, a LUMO energy level, a T1 energy level, and an S1 energy level that are appropriate for use as a material for an organic layer (for example, a material for the EML) disposed between a pair of electrodes of an organic light-emitting device, and have good thermal and electrical stabilities. For example, when the condensed cyclic compound of Formula 1 above is used as a host in the EML of an organic light-emitting device, the organic light-emitting device may have high efficiency and long lifetime, based on the host-dopant energy transfer mechanism.

Although not limited to any specific theory, Compound B below may have too strong electron transport ability to achieve an equilibrium between hole transport and electron transport. Accordingly, an organic light-emitting device including Compound B may have poor efficiency characteristics. Compound C below includes a condensed cyclic core in a pyrazine ring, instead of a pyrimidine ring, and thus may have poor thermal and electrical stabilities.

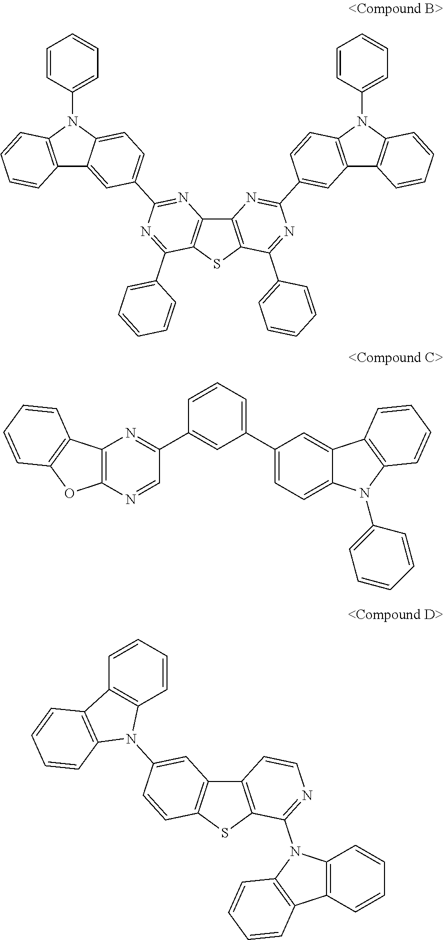 [SMILES: c1ccc(-c2nc(-c3ccc4c(c3)c3ccccc3n4-c3ccccc3)nc3c2sc2c(-c4ccccc4)nc(-c4ccc5c(c4)c4ccccc4n5-c4ccccc4)nc23)cc1 ; c1ccc(-n2c3ccccc3c3cc(-c4cccc(-c5cnc6oc7ccccc7c6n5)c4)ccc32)cc1 ; c1ccc2c(c1)c1ccccc1n2-c1ccc2sc3c(-n4c5ccccc5c5ccccc54)nccc3c2c1]

The HOMO, LUMO, and triplet (T1) energy levels of Compounds 30, 29, 27, b-41, b-71, b-116, a-30, a-40, a-41, a-42, a-46, a-56, a-70, a-71, a-74, a-75, a-82, a-84, a-108, a-110, a-112, a-114, a-116, e-70, e-71, e-74, e-82, e-84, e-88, e-114, f-70, f-71, f-74, f-75, f-82, f-84, f-88, and f-114, and Compounds B, C and D were measured using Gaussian simulation (each energy level of the materials is calculated using Supercomputer GAIA (IBM power 6) with Gaussian 09 method). The results are shown in Table 1 below.

TABLE 1

| Compound | HOMO | LUMO | T1 energy level | S1 energy level |
|---|---|---|---|---|
| No. | (eV) | (eV) | (eV) | (eV) |
| 30 | −5.649 | −1.804 | 2.756 | 3.503 |
| 29 | −5.615 | −1.819 | 2.745 | 3.404 |
| 27 | −5.714 | −1.819 | 2.762 | 3.536 |
| b-41 | −5.712 | −1.920 | 2.838 | 3.455 |
| b-71 | −5.871 | −1.926 | 2.837 | 3.523 |
| b-116 | −5.806 | −1.909 | 2.494 | 3.424 |
| a-30 | −5.732 | −1.970 | 2.645 | 3.334 |
| a-40 | −5.736 | −1.806 | 2.857 | 3.522 |
| a-41 | −5.737 | −1.836 | 2.849 | 3.509 |
| a-42 | −5.727 | −1.881 | 2.771 | 3.452 |
| a-46 | −5.734 | −1.825 | 2.834 | 3.507 |
| a-56 | −5.722 | −1.814 | 2.841 | 3.503 |
| a-70 | −5.847 | −1.798 | 2.861 | 3.531 |
| a-71 | −5.849 | −1.806 | 2.85 | 3.525 |
| a-74 | −5.85 | −1.779 | 2.863 | 3.529 |
| a-75 | −5.824 | −1.798 | 2.848 | 3.505 |
| a-82 | −5.75 | −1.798 | 2.861 | 3.532 |
| a-84 | −5.741 | −1.804 | 2.846 | 3.526 |
| a-114 | −5.735 | −1.8 | 2.85 | 3.523 |
| a-108 | −5.582 | −1.813 | 2.283 | 3.295 |
| a-110 | −5.601 | −1.827 | 2.663 | 3.43 |
| a-112 | −5.577 | −1.827 | 2.648 | 3.41 |
| a-116 | −5.747 | −1.81 | 2.864 | — |
| e-70 | −5.881 | −1.852 | 2.690 | 3.650 |
| e-71 | −5.919 | −1.853 | 2.690 | 3.687 |
| e-74 | −5.932 | −1.836 | 2.692 | 3.727 |
| e-75 | −5.872 | −1.851 | 2.686 | 3.623 |
| e-82 | −5.757 | −1.850 | 2.693 | 3.612 |
| e-84 | −5.771 | −1.870 | 2.689 | 3.616 |
| e-88 | −5.871 | −1.884 | 2.691 | 3.617 |
| e-114 | −5/63 | −1.863 | 2.690 | 3.606 |
| f-70 | −5.869 | −1.832 | 2.716 | 3.655 |
| f-71 | −5.875 | −1.836 | 2.716 | 3.658 |
| f-74 | −5.886 | −1.832 | 2.689 | 3.636 |
| f-75 | −5.949 | −1.811 | 2.703 | 3.735 |
| f-82 | −5.758 | −1.837 | 2.714 | 3.642 |
| f-84 | −5.753 | −1.844 | 2.715 | 3.635 |
| f-88 | −5.867 | −1.838 | 2.715 | 3.656 |
| f-114 | −5.746 | −1.836 | 2.714 | 3.625 |
| B | −5.302 | −2.145 | 2.705 | — |
| C | −5.392 | −1.660 | 2.866 | — |
| D | −5.501 | −1.563 | 2.684 | — |

Referring to Table 1, the absolute value of the LUMO energy level of Compound B was greater than the absolute values of the LUMO energy levels of Compounds 30, 29, 27, b-41, b-71, b-116, a-30, a-40, a-41, a-42, a-46, a-56, a-70, a-71, a-74, a-75, a-82, a-84, a-108, a-110, a-112, a-114, a-116, e-70, e-71, e-74, e-82, e-84, e-88, e-114, f-70, f-71, f-74, f-75, f-82, f-84, f-88, and f-114, indicating too strong electron transport ability of Compound B. The absolute values of the LUMO energy levels of Compounds C and D were smaller than those of Compounds 30, 29, 27, b-41, b-71, b-116, a-30, a-40, a-41, a-42, a-46, a-56, a-70, a-71, a-74, a-75, a-82, a-84, a-108, a-110, a-112, a-114, a-116, e-70, e-71, e-74, e-82, e-84, e-88, e-114, f-70, f-71, f-74, f-75, f-82, f-84, f-88, and f-114, indicating too weak electron transport ability of Compounds C and D. Accordingly, Compounds B, C and D were found to be less likely to achieve equilibrium between hole transport and electron transport, compared to Compounds 30, 29, 27, b-41, b-71, b-116, a-30, a-40, a-41, a-42, a-46, a-56, a-70, a-71, a-74, a-75, a-82, a-84, a-108, a-110, a-112, a-114, a-116, e-70, e-71, e-74, e-82, e-84, e-88, e-114, f-70, f-71, f-74, f-75, f-82, f-84, f-88, and f-114.

A synthesis method of the condensed cyclic compound of Formula 1 above may be easily understood to one of ordinary skill in the art based on the synthesis examples described below.

As described above, the condensed cyclic compound of Formula 1 above may be appropriate for use as a host or as a hole transport auxiliary layer of the EML of the organic layer (a host of the EML).

Due to the inclusion of the organic layer including the condensed cyclic compound of Formula 1 described above, the organic light-emitting device may have a low driving voltage, a high efficiency, and a long lifetime.

The condensed cyclic compound of Formula 1 above may be used between a pair of electrodes of an organic light-emitting device. For example, the condensed cyclic compound of Formula 1 above may be included in at least one of the EML, a hole transport region between the first electrode and the EML (for example, the hole transport region may include at least one of a hole injection layer (HIL), a hole transport layer (HTL), and an electron blocking layer (EBL)), and an electron transport region between the EML and the second electrode (for example, the electron transport region may include at least one of a hole blocking layer (HBL), an electron transport layer (ETL), and an electron injection layer (EIL). For example, the condensed cyclic compound of Formula 1 above may be included in the EML, wherein the EML may further include a dopant, and the condensed cyclic compound of Formula 1 in the EML may serve as a host. For example, the EML may be a green EML, and the dopant may be a phosphorescent dopant.

As used herein, “(for example, the organic layer) including at least one condensed cyclic compound means that “(the organic layer) including one of the condensed cyclic compounds of Formula 1 above, or at least two different condensed cyclic compounds of Formula 1 above”.

For example, the organic layer of the organic light-emitting device may include only Compound 1 as the condensed cyclic compound. For example, Compound 1 may be included in the EML of the organic light-emitting device. In some embodiments, the organic layer of the organic light-emitting device may include Compounds 1 and 2 as the condensed cyclic compound. For example, Compounds 1 and 2 may be included in the same layer (for example, in the EML) or in different layers. For example, the condensed cyclic compound may be included as a host in an emission of an organic layer, or in an electron transport auxiliary layer.

For example, the first electrode may be an anode, the second electrode may be a cathode, and the organic layer may include i) a hole transport region disposed between the first electrode and the emission layer and comprising at least one of a hole injection layer, a hole transport layer, and an electron blocking layer; and ii) an electron transport region disposed between the emission layer and the second electrode and including at least one of a hole blocking layer, an electron transport layer, and an electron injection layer.

The term “organic layer” as used herein refers to a single layer and/or a plurality of layers disposed between the first and second electrodes of the organic light-emitting device. The “organic layer” may include, for example, an organic compound or an organometallic complex including a metal.

According to another embodiment of the present disclosure, an organic light-emitting device includes a first electrode, a second electrode, and an organic layer disposed between the first electrode and the second electrode and including an EML and at least one of the condensed cyclic compounds of Formula 1 above.

FIGS. 1 to 3 are schematic views of an organic light-emitting device 10 according to an embodiment of the present disclosure. Hereinafter, a structure of an organic light-emitting device according to an embodiment of the present disclosure and a method of manufacturing the same will now be described with reference to FIG. 1. Referring to FIG. 1, the organic light-emitting device 10 has a structure in which a substrate, a first electrode 11, an organic layer 15, and a second electrode 19 are sequentially stacked in this order.

A substrate (not shown) may be disposed under the first electrode 11 or on the second electrode 19 in FIG. 1. The substrate may be any substrate that is used in conventional organic light emitting devices. In some embodiments the substrate may be a glass substrate or a transparent plastic substrate with strong mechanical strength, thermal stability, transparency, surface smoothness, ease of handling, and water resistance.

The first electrode 11 may be formed by depositing or sputtering a first electrode-forming material on the substrate. The first electrode 11 may be an anode. A material having a high work function may be selected as a material for the first electrode to facilitate hole injection. The first electrode 11 may be a reflective electrode, a semi-transmissive electrode, or a transmissive electrode. For example, the material for the first electrode 11 may be indium tin oxide (ITO), indium zinc oxide (IZO), tin oxide (SnO₂), or zinc oxide (ZnO). In some embodiments, the material for the first electrode 11 may be metals, for example, magnesium (Mg), aluminum (Al), aluminum-lithium (Al—Li), calcium (Ca), magnesium-indium (Mg—In), magnesium-silver (Mg—Ag), or the like.

The first electrode 11 may have a single-layer structure or a multi-layer structure including at least two layers.

The organic layer 15 may be disposed on the first electrode 11.

The organic layer 15 may include at least one a hole transport region; an EML, and an electron transport region.

The hole transport region may be disposed between the first electrode 11 and the EML.

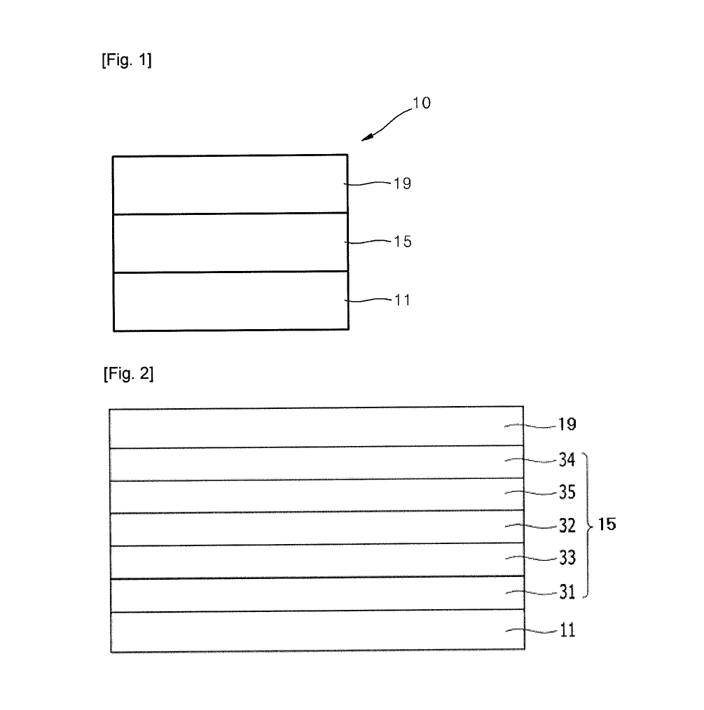

The hole transport region may include at least one of a hole injection layer (HIL), a hole transport layer (HTL), an electron blocking layer (EBL), and a buffer layer. For example, referring to FIG. 2, an organic light-emitting device according to one embodiment is described as follows.

The organic layer 15 includes a hole transport layer 31, an emission layer 32, and a hole transport auxiliary layer 33 interposed between the hole transport layer 31 and the emission layer 32.

The hole transport region may include at least two hole transport layers, and a hole transport layer contacting the emission layer is defined to be a hole transport auxiliary layer.

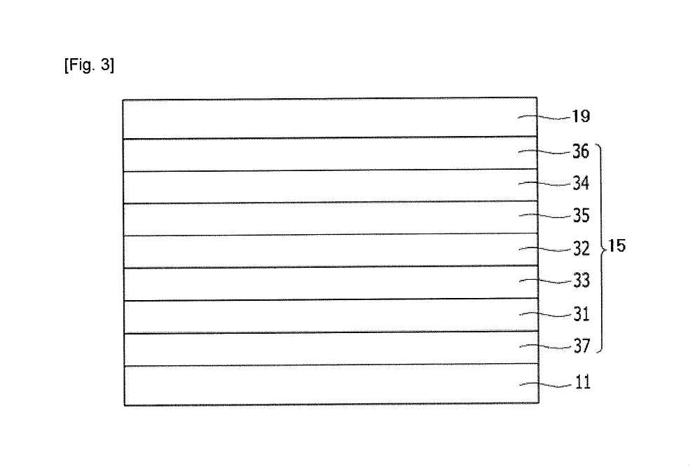

The hole transport region may include exclusively the HIL or the HTL. In some embodiments, the electron transport region may have a structure including a HIL 37/HTL 31 or a HIL 37/HTL 31/EBL, wherein the layers forming the structure of the electron transport region may be sequentially stacked on the first electrode 11 in the stated order. For example, a hole injection layer 37 and an electron injection layer 36 are additionally included and thus a first electrode 11/hole injection layer 37/hole transport layer 31/hole transport auxiliary layer 33/emission layer 32/electron transport auxiliary layer 35/electron transport layer 34/electron injection layer 36/a second electrode 19 are sequentially stacked, as shown in FIG. 3.

The hole injection layer 37 may improve interface properties between ITO as an anode and an organic material used for the hole transport layer 31, and is applied on a non-planarized ITO and thus planarizes the surface of the ITO. For example, the hole injection layer 37 may include a material having a median value, particularly desirable conductivity between a work function of ITO and HOMO of the hole transport layer 31, in order to adjust a difference a work function of ITO as an anode and HOMO of the hole transport layer 31. In connection with the present disclosure, the hole injection layer 37 may include N4,N4′-diphenyl-N4,N4′-bis(9-phenyl-9H-carbazol-3-yl)biphenyl-4,4′-diamine), but is not limited thereto. In addition, the hole injection layer 37 may further include a conventional material, for example, copper phthalocyanine (CuPc), aromatic amines such as N,N′-dinaphthyl-N,N′-phenyl-(1,1′-biphenyl)-4,4′-diamine, NPD), 4,4′,4″-tris[methylphenyl(phenyl)amino]triphenyl amine (m-MTDATA), 4,4′,4″-tris[1-naphthyl(phenyl)amino]triphenyl amine (1-TNATA), 4,4′,4″-tris[2-naphthyl(phenyl)amino]triphenyl amine (2-TNATA), 1,3,5-tris[N-(4-diphenylaminophenyl)phenylamino]benzene (p-DPA-TDAB), and the like, compounds such as 4,4′-bis[N-[4-{N,N-bis(3-methylphenyl)amino}phenyl]-N-phenylamino]biphenyl (DNTPD), hexaazatriphenylene-hexacarbonitrile (HAT-CN), and the like, a polythiophene derivative such as poly(3,4-ethylenedioxythiophene)-poly(styrenesulfonate) (PEDOT) as a conductive polymer. The hole injection layer 37 may be, for example coated on ITO as an anode in a thickness of 10 to 300 Å.

The electron injection layer 36 is stacked on the electron transport layer to facilitate electron injection into a cathode and improves power efficiency. The electron injection layer 36 may include any generally-used material in this art without limitation, for example, LiF, Liq, NaCl, CsF, Li₂O, BaO, and the like.

When the hole transport region includes the HIL, the HIL may be formed on the first electrode 11 by any of a variety of methods, for example, vacuum deposition, spin coating, casting, Langmuir-Blodgett (LB) deposition, or the like.

When the HIL is formed using vacuum deposition, vacuum deposition conditions may vary depending on the material that is used to form the HIL, and the desired structure and thermal properties of the HIL to be formed. For example, vacuum deposition may be performed at a temperature of about 100□ to about 500□, a pressure of about 10⁻⁸ torr to about 10⁻³ torr, and a deposition rate of about 0.01 to about 100 Å/sec. However, the deposition conditions are not limited thereto.

When the HIL is formed using spin coating, the coating conditions may vary depending on the material that is used to form the HIL, and the desired structure and thermal properties of the HIL to be formed. For example, the coating rate may be in the range of about 2000 rpm to about 5000 rpm, and a temperature at which heat treatment is performed to remove a solvent after coating may be in a range of about 80□, to about 200□. However, the coating conditions are not limited thereto.

Conditions for forming the HTL and the EBL may be defined based on the above-described formation conditions for the HIL.

In some embodiments, the hole transport region may include at least one of m-MTDATA, TDATA, 2-TNATA, NPB, β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzene sulfonic acid (Pani/DBSA), poly(3,4-ethylenedioxythiophene)/poly(4-styrenesulfonate)(PEDOT/PSS), polyaniline/camphor sulfonic acid (Pani/CSA), polyaniline/poly(4-styrenesulfonate) (PANI/PSS), a compound represented by Formula 201 below, and a compound represented by Formula 202 below.

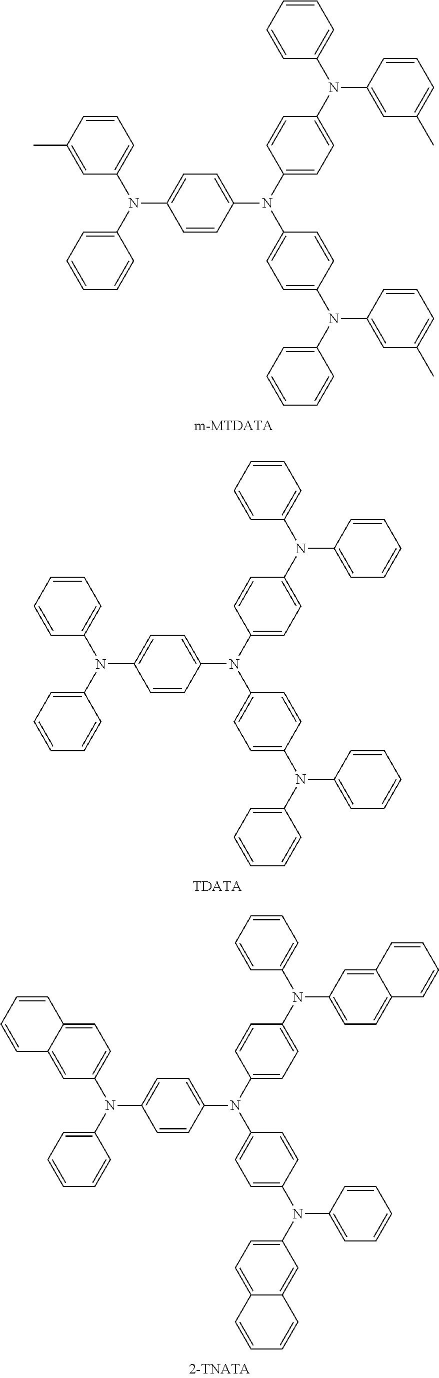

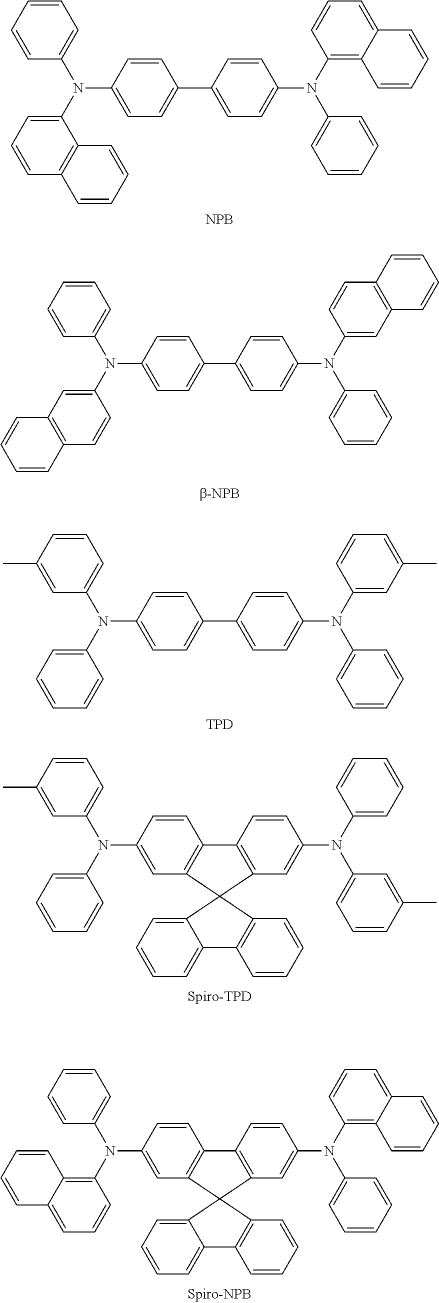

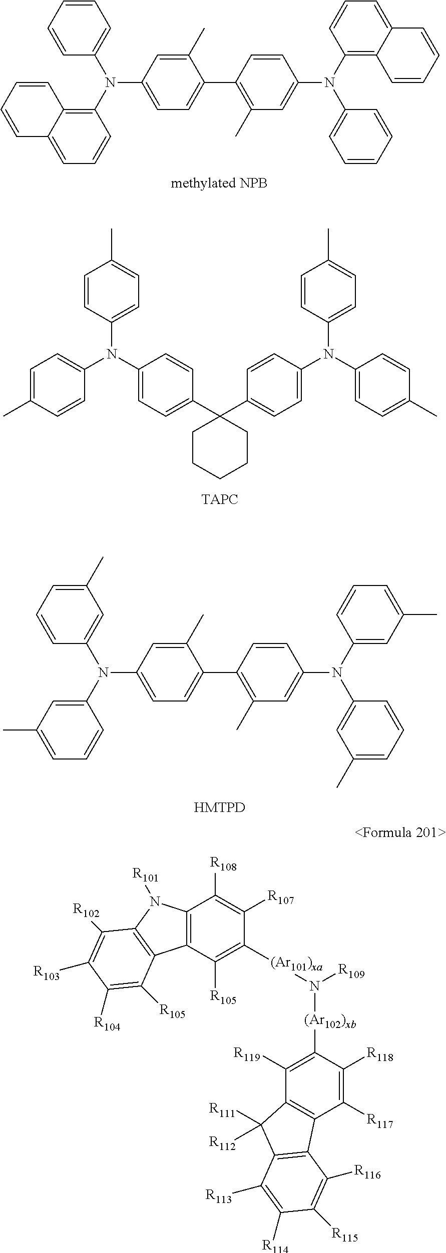

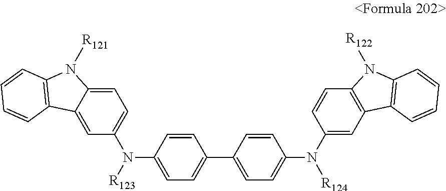

In Formula 201 above, Ar₁₀₁ and Ar₁₀₂ may be each independently selected from

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, and a pentacenylene group, and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, and a pentacenylene group, each substituted with at least one of a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

In Formula 201, xa and xb may be each independently an integer from 0 to 5, for example, may be 0, 1, or 2. For example, xa may be 1, and xb may be 0, but are not limited thereto.

In Formulae 201 and 202, R₁₀₁ to R₁₀₈, R₁₁₁ to R₁₁₉, and R₁₂₁ to R₁₂₄ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, or the like), and a C₁-C₁₀ alkoxy group (for example, a methoxy group, an ethoxy group, a propoxy group, a butoxy group, a pentoxy group, or the like);

a C₁-C₁₀ alkyl group and a C₁-C₁₀ alkoxy group, each substituted with at least one of a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one of a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group. However, embodiments of the present disclosure are not limited thereto.

In Formula 201 above, R₁₀₉ may be selected from

a phenyl group, a naphthyl group, an anthracenyl group, and a pyridinyl group, and

a phenyl group, a naphthyl group, an anthracenyl group, and a pyridinyl group, each substituted with at least one of a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group.

In some embodiments, the compound of Formula 201 may be represented by Formula 201A, but is not limited thereto:

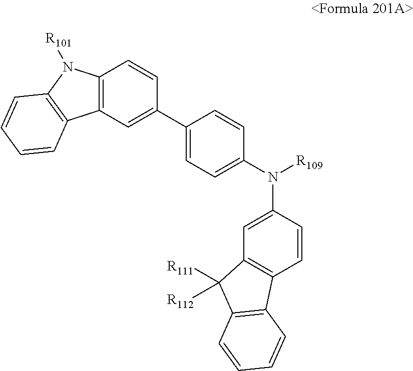

In Formula 201A, R₁₀₁, R₁₁₁, R₁₁₂, and R₁₀₉ may be the same as those defined above.

For example, the compound of Formula 201 and the compound of Formula 202 may include Compounds HT1 to HT20 below, but are not limited thereto:

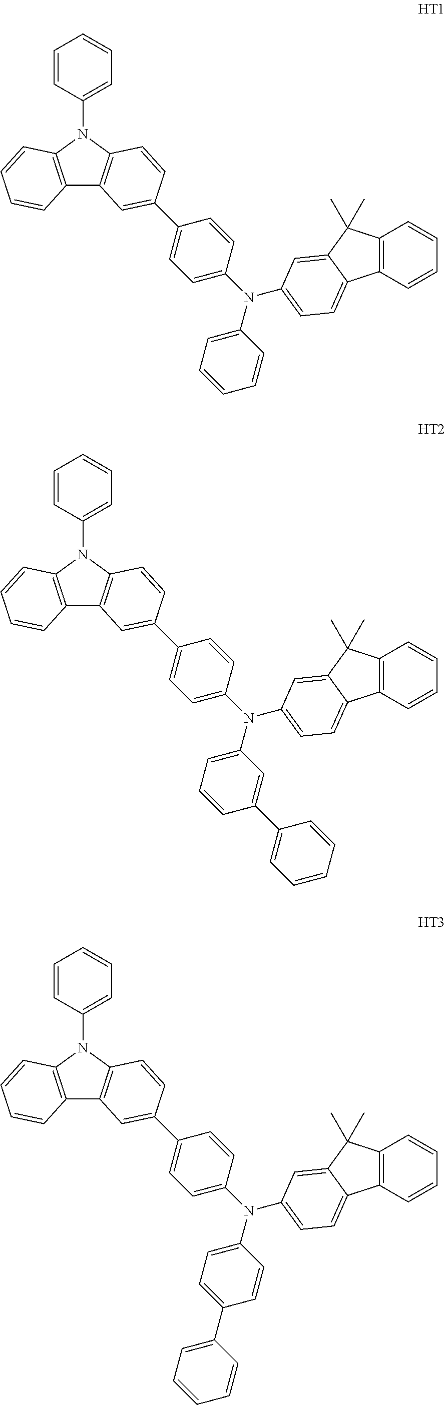 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

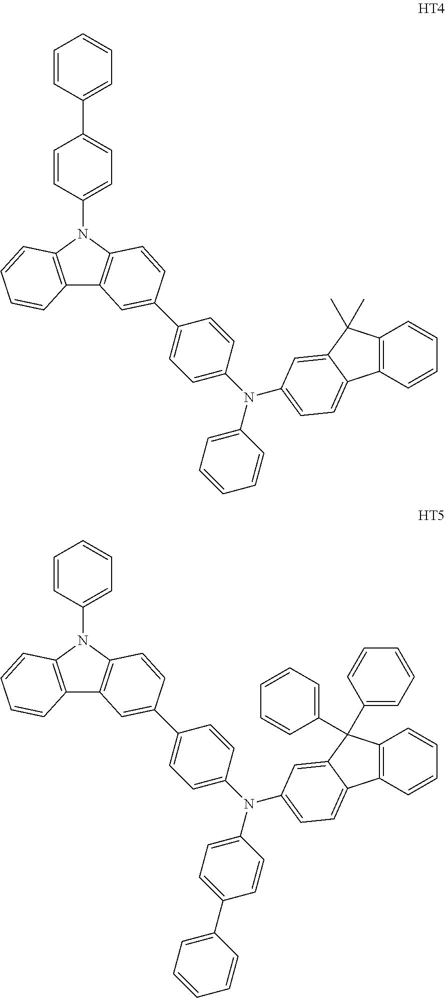 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

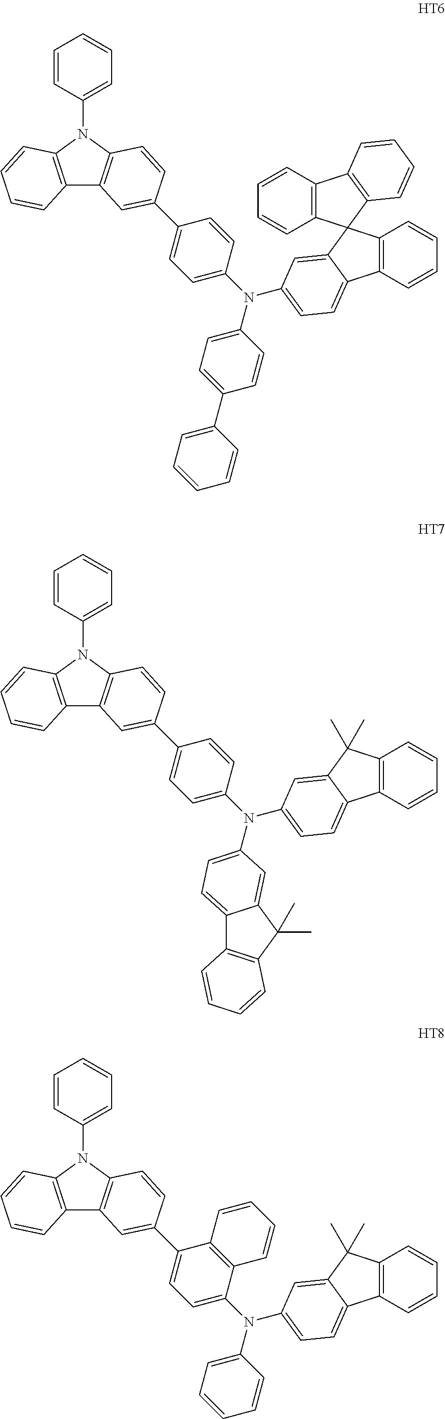 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

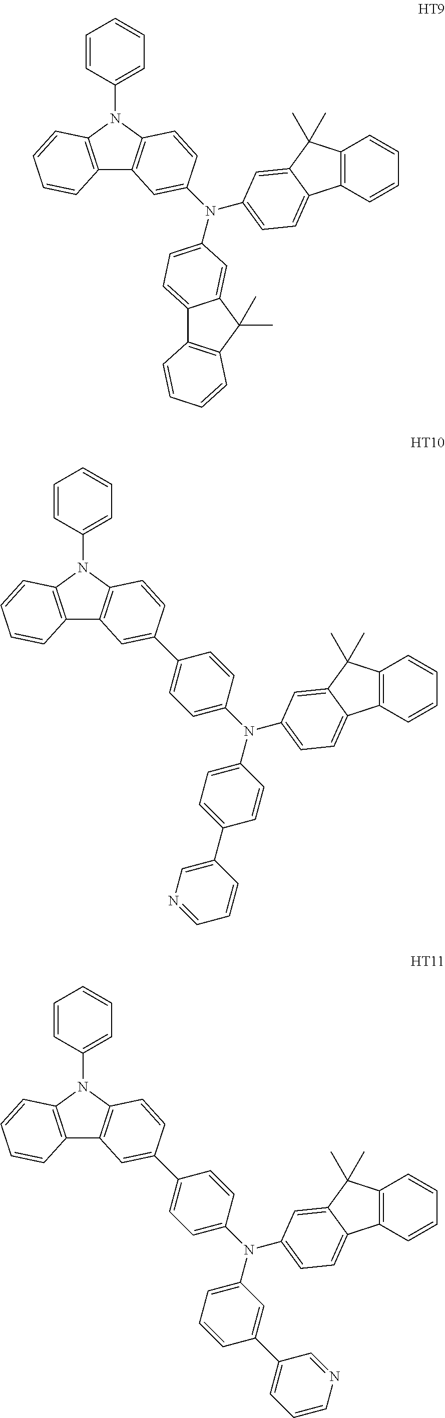 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

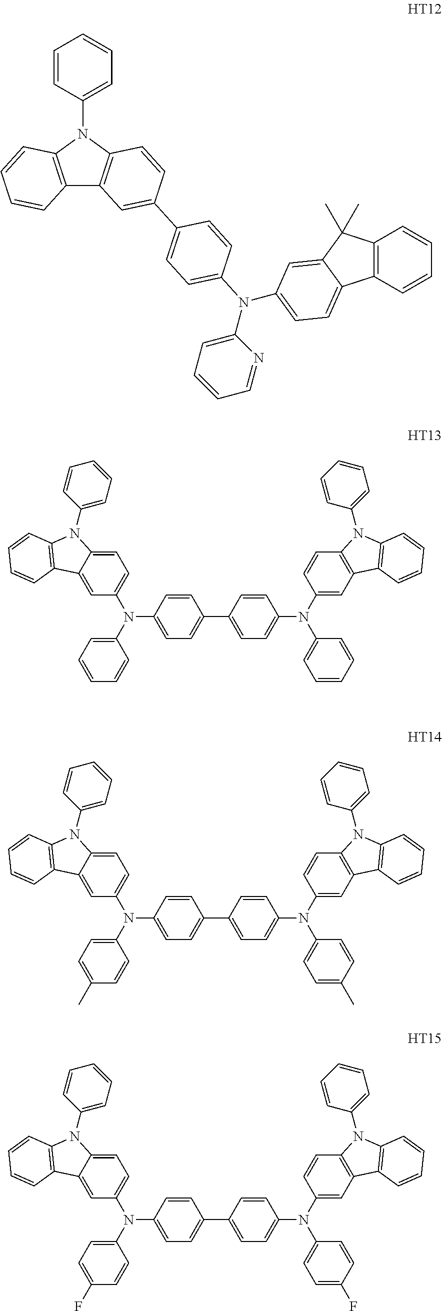 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

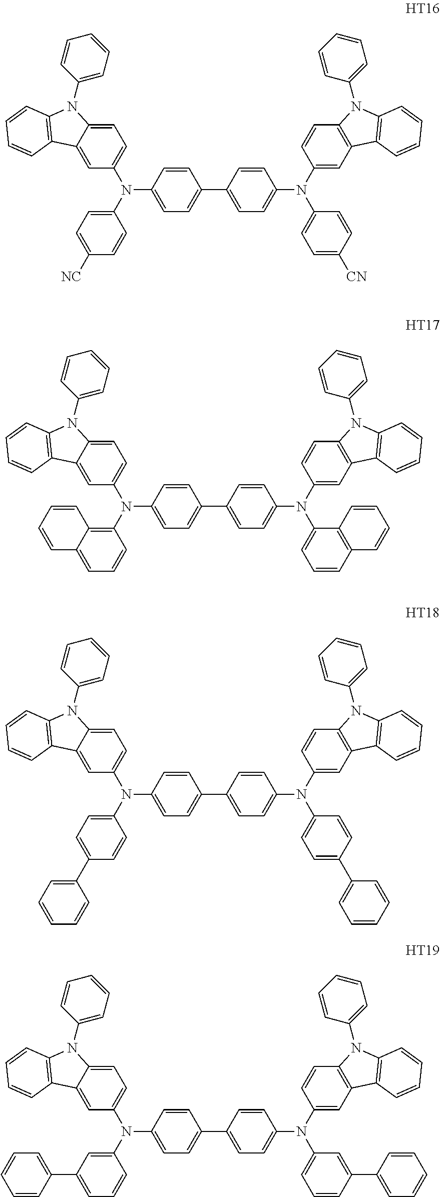 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

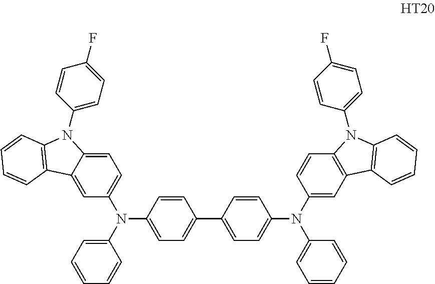 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be from about 100 Å to about 10000 Å, and in some embodiments, from about 100 Å to about 1000 Å. When the hole transport region includes a HIL and a HTL, a thickness of the HIL may be from about 100 Å to about 10,000 Å, and in some embodiments, from about 100 Å to about 1,000 Å, and a thickness of the HTL may be from about 50 Å to about 2,000 Å, and in some embodiments, from about 100 Å to about 1,500 Å. When the thicknesses of the hole transport region, the HIL, and the HTL are within these ranges, satisfactory hole transport characteristics may be obtained without a substantial increase in driving voltage.

The hole transport region may further include a charge-generating material to improve conductivity, in addition to the materials as described above. The charge-generating material may be homogeneously or inhomogeneously dispersed in the hole transport region.

The charge-generating material may be, for example, a p-dopant. The p-dopant may be one of a quinine derivative, a metal oxide, and a cyano group-containing compound, but is not limited thereto. Non-limiting examples of the p-dopant are quinone derivatives such as tetracyanoquinonedimethane (TCNQ), 2,3,5,6-tetrafluoro-tetracyano-1,4-benzoquinonedimethane (F4-TCNQ), and the like; metal oxides such as tungsten oxide, molybdenum oxide, and the like; and cyano-containing compounds such as Compound 200 below.

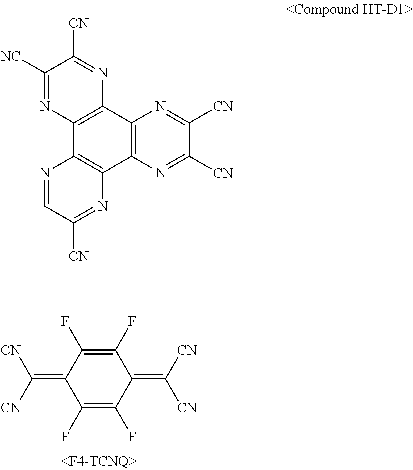

The hole transport region may further include a buffer layer.

The buffer layer may compensate for an optical resonance distance of light according to a wavelength of the light emitted from the EML, and thus may increase efficiency.

The EML may be formed on the hole transport region by using vacuum deposition, spin coating, casting, LB deposition, or the like. When the EML is formed using vacuum deposition or spin coating, the conditions for deposition and coating may be similar to those for the formation of the HIL, though the conditions for the deposition and coating may vary depending on the material that is used to form the EML.

The EML may include a host and a dopant. The host may include at least one of the condensed cyclic compounds of Formula 1 above. For example, the host may include the first host and the second host, which may be different from each other.

In some embodiments, the organic layer of the organic light-emitting device may include only the above condensed compound (the first host), or further include at least one of a first compound represented by Formula 41 below and a second compound represented by Formula 61 below, in addition to the condensed cyclic compound of Formula 1 above.

The second host may include at least one of the first compound represented by Formula 41 and the second compound represented by Formula 61. The ring A₆₁ is represented by the following Formula 61A, and the ring A₆₂ is represented by the following Formula 61B. In Formula 61 below, the ring A₆₁ is fused to an adjacent 5-membered ring and the ring A₆₂ with sharing carbons therewith, and the ring A₆₂ is fused to the adjacent ring A₆₁ and a 6-membered ring with sharing carbons therewith:

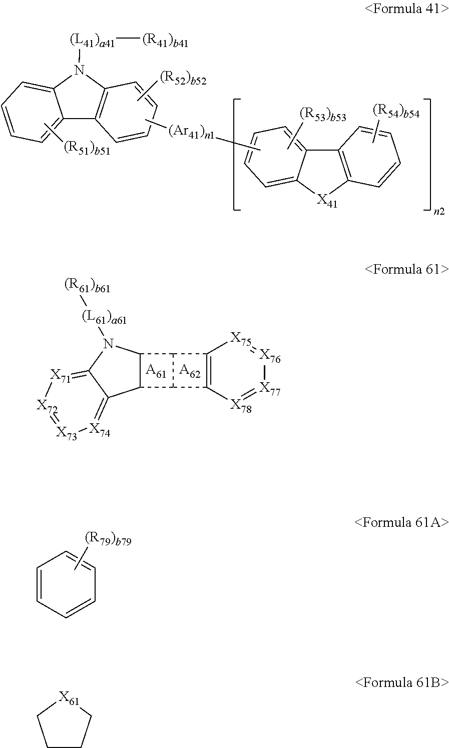

In Formulae 41 and 61 above,

X₄₁ may be N-[(L₄₂)ₐ₄₂₋(R₄₂)_{b42}], S, O, S(═O), S(═O)₂, a C(═O), a C(R₄₃)(R₄₄), Si(R₄₃)(R₄₄), P(R₄₃), P(═O)(R₄₃), or C═N(R₄₃);

ring A₆₁ in Formula 61 may be represented by Formula 61A above;

ring A₆₂ in Formula 61 may be represented by Formula 61B above;

X₆₁ may be N-[(L₆₂)ₐ₆₂-(R₆₂)_{b62}], S, O, S(═O), S(═O)₂, a C(═O), a C(R₆₃)(R₆₄), Si(R₆₃)(R₆₄), P(R₆₃), P(═O)(R₆₃), or C═N(R₆₃),

X₇₁ may be C(R₇₁) or N; X₇₂ may be C(R₇₂) or N; X₇₃ may be C(R₇₃) or N; X₇₄ may be C(R₇₄) or N; X₇₅ may be C(R₇₅) or N; X₇₆ may be C(R₇₆) or N; X₇₇ may be C(R₇₇) or N; X₇₈ may be C(R₇₈) or N;

Ar₄₁, L₄₁, L₄₂, L₆₁, and L₆₂ may be each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;

n1 and n2 may be each independently an integer selected from 0 to 3;

R₄₁ to R₄₄, R₅₁ to R₅₄, R₆₁ to R₆₄, and R₇₁ to R₇₉ may be each independently selected from a hydrogen, a deuterium a fluoro group (—F), a chloro group (—Cl), a bromo group (—Br), an iodo group (—I), a hydroxyl group, a cyano group, an amino group, an amidino group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), and —B(Q₆)(Q₇); and

a41, a42, a61, and a62 may be each independently an integer selected from 0 to 3;

b41, b42, b51 to b54, b61, b62, and b79 may be each independently an integer selected from 1 to 3.

In some embodiments, in Formulae 41 and 61 above, R₄₁ to R₄₄, R₅₁ to R₅₄, R₆₁ to R₆₄ and R₇₁ to R₇₉ may be each independently selected from

a hydrogen, a deuterium atom, —F, —Cl, —Br, —I, a hydroxyl group, a cyano group, an amino group, an amidino group, a C₁-C₂₀ alkyl group, a C₁-C₂₀ alkoxy group, a C₃-C₁₀ cycloalkyl group, a C₃-C₁₀ cycloalkenyl group, a C₆-C₂₀ aryl group, and a monovalent non-aromatic condensed polycyclic group,

In some other embodiments, in Formulae 41 and 61 above, R₄₁ to R₄₄, R₅₁ to R₅₄, R₆₁ to R₆₄ and R₇₁ to R₇₉ may be each independently selected from

a hydrogen atom, a deuterium atom, —F, —Cl, —Br, —I, a hydroxyl group, a cyano group, an amino group, an amidino group, a C₁-C₂₀ alkyl group, and a C₁-C₂₀ alkoxy group;

a phenyl group, a pentalenyl group, a naphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a picenyl group, a perylenyl group, a pentaphenyl group, a carbazolyl group, a benzofuranyl group, a benzothiophenyl group, a dibenzofuranyl group, a dibenzothiophenyl group, a benzocarbazolyl group, and a dibenzocarbazolyl group; and

a phenyl group, a pentalenyl group, a naphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a picenyl group, a perylenyl group, a pentaphenyl group, a carbazolyl group, a benzofuranyl group, a benzothiophenyl group, a dibenzofuranyl group, a dibenzothiophenyl group, a benzocarbazolyl group, and a dibenzocarbazolyl group, each substituted with at least one selected from a deuterium, —F, —Cl, —Br, —I, a hydroxyl group, a cyano group, an amino group, an amidino group, a C₁-C₂₀ alkyl group, a C₁-C₂₀ alkoxy group, a phenyl group, a pentalenyl group, a naphthyl group, a fluorenyl group, a spiro-fluorenyl group, a benzofluorenyl group, a dibenzofluorenyl group, a phenalenyl group, a phenanthrenyl group, an anthracenyl group, a fluorantenyl group, a triphenylenyl group, a pyrenyl group, a chrysenyl group, a picenyl group, a perylenyl group, a pentaphenyl group, a carbazolyl group, a benzofuranyl group, a benzothiophenyl group, a dibenzofuranyl group, a dibenzothiophenyl group, a benzocarbazolyl group, and a dibenzocarbazolyl group, but are not limited thereto.

For example, the L₆₁ and L₆₂ are each independently a substituted or unsubstituted C₆-C₆₀ arylene group, a substituted or unsubstituted C₂-C₆₀ heteroarylene group, or a substituted or unsubstituted divalent non-aromatic condensed polycyclic group, R₅₁ to R₅₄, R₆₁ to R₆₄ and R₇₁ to R₇₉ are each independently hydrogen, a deuterium, —F, —Cl, —Br, —I, a hydroxyl group, a cyano group, an amino group, an amidino group, a substituted or unsubstituted C₁-C₂₀ alkyl group, a substituted or unsubstituted C₁-C₂₀ alkoxy group, a substituted or unsubstituted C₃-C₁₀ cycloalkyl group, a substituted or unsubstituted C₃-C₁₀ cycloalkenyl group, a substituted or unsubstituted C₆-C₂₀ aryl group, or a substituted or unsubstituted monovalent non-aromatic condensed heteropolycyclic group.

In some embodiments, R₅₁, R₅₃, and R₅₄ in Formula 41, and R₇₁ to R₇₉ in Formula 61 may be each independently selected from a hydrogen, a deuterium, —F, —Cl, —Br, —I, a hydroxyl group, a cyano group, a nitro group, an amino group, an amidino group, a C₁-C₂₀ alkyl group, a C₂-C₂₀ alkenyl group, a C₂-C₂₀ alkynyl group, and a C₁-C₂₀ alkoxy group.

In some other embodiments, R₅₁, R₅₃, and R₅₄ in Formula 41, and R₇₁ to R₇₉ in Formula 61 may be all hydrogens.

R₄₁, R₄₂, and R₅₂ in Formula 41, and R₆₁ and R₆₂ in Formula 61 may be each independently a group represented by one of Formulae 4-1 to 4-33 above.

In some embodiments, R₄₁, R₄₂, and R₅₂ in Formula 41, and R₆₁ and R₆₂ in Formula 61 may be each independently a group represented by one of Formulae 4-1 to 4-5, and Formulae 4-26 to 4-33 regarding Formula 1 above.

In some other embodiments, R₄₁, R₄₂, and R₅₂ in Formula 41, and R₆₁ and R₆₂ in Formula 61 may be each independently a group represented by one of Formulae 5-1 to 5-27, and Formulae 5-40 to 5-44 regarding Formula 1 above. However, embodiments of the present disclosure are not limited thereto.

According to another embodiment, an organic light-emitting device includes the emission layer including the first host, the second host and a dopant, wherein the first host and the second host are different from each other,

the first host including the condensed cyclic compound represented by Formula 1, and

the second host including at least one of the first compound represented by the following Formula 41 and the second compound represented by the following Formula 61.

In some other embodiments, the first compound of Formula 41 above may be represented by one of Formulae 41-1 to 41-12 below, and the second compound of Formula 61 above may be represented by one of Formulae 61-1 to 61-6 below.

However, embodiments of the present disclosure are not limited thereto.

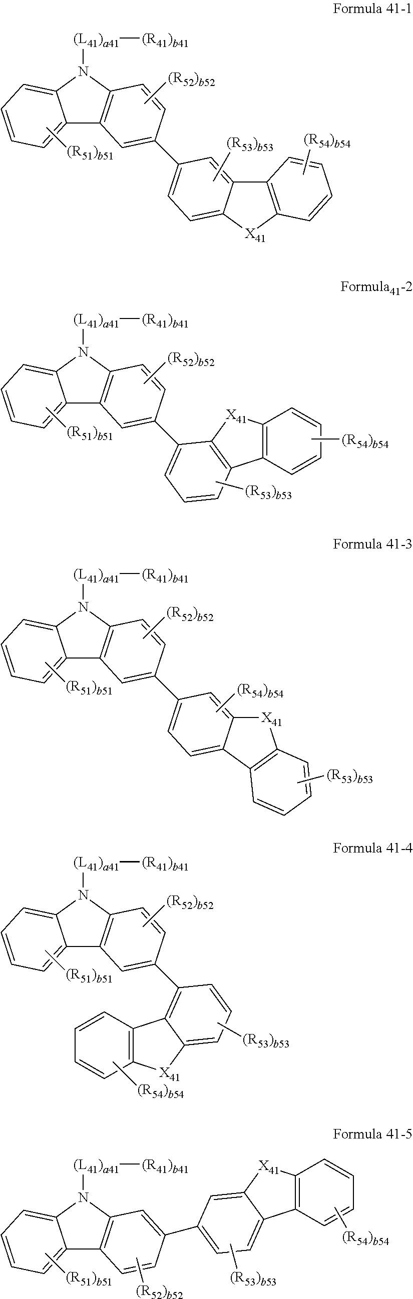

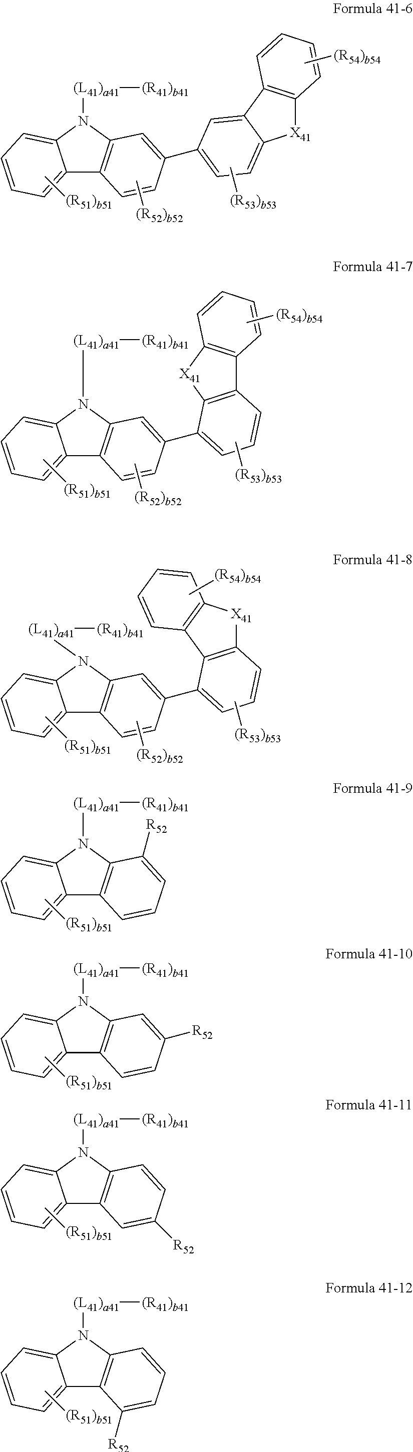

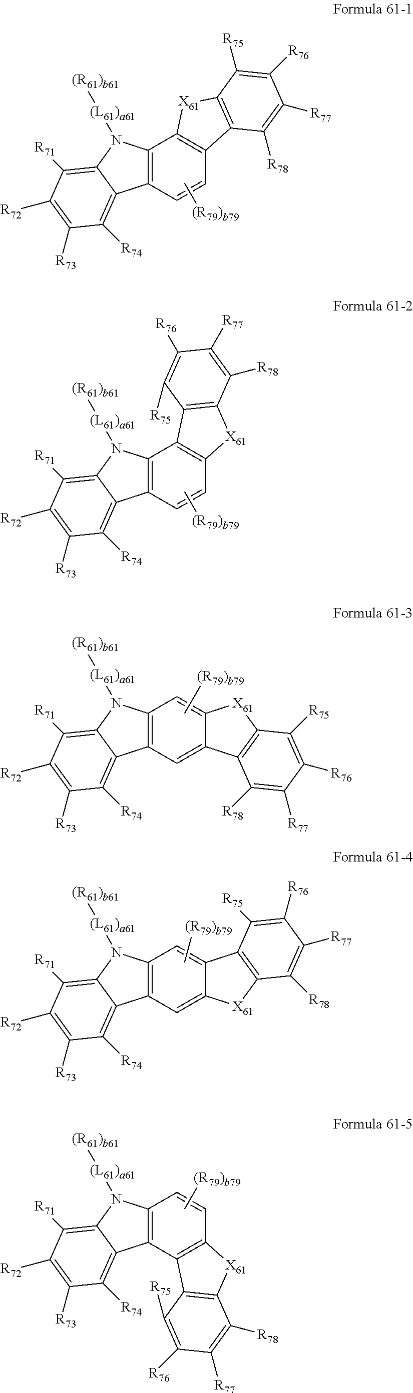

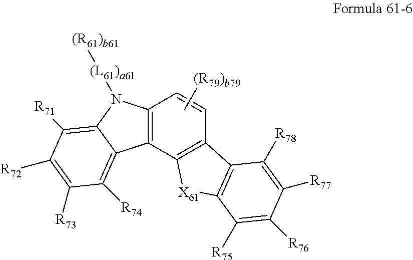

In Formulae 41-1 to 41-12, and Formulae 61-1 to 61-6, X₄₁, X₆₁, L₄₁, a41, L₆₁, a61, R₄₁, R₅₁ to R₅₄, b41, b51 to b54, R₆₁, b61, R₇₁ to R₇₉, and b79 may be the same as those defined above.

The condensed cyclic compound represented by Formula 1 includes one of the compounds of Group I,

in some embodiments, the first compound of Formula 41 above may include one of Compounds A1 to A111 below, and the second compound of Formula 61 may include one of Compounds B1 to B20 below. However, embodiments of the present disclosure are not limited thereto.

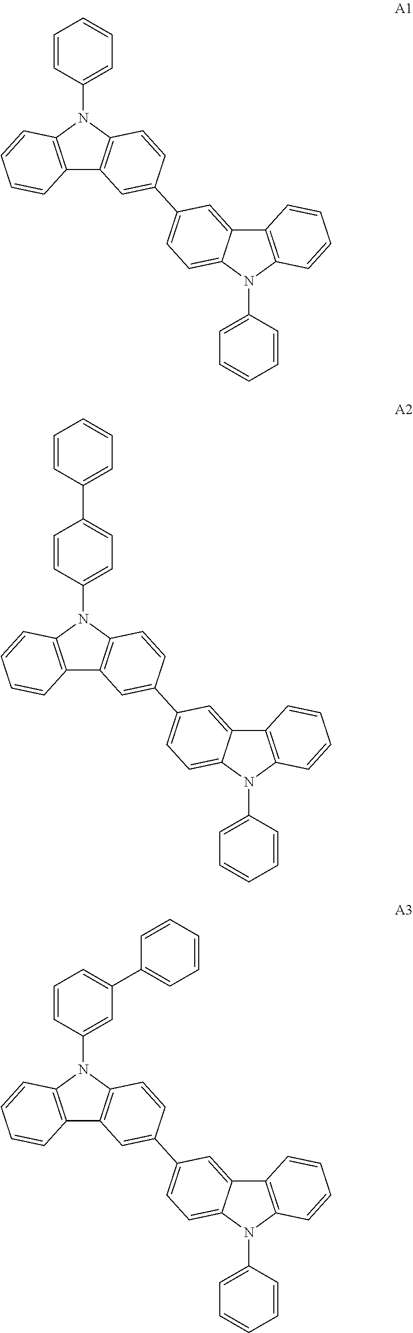 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

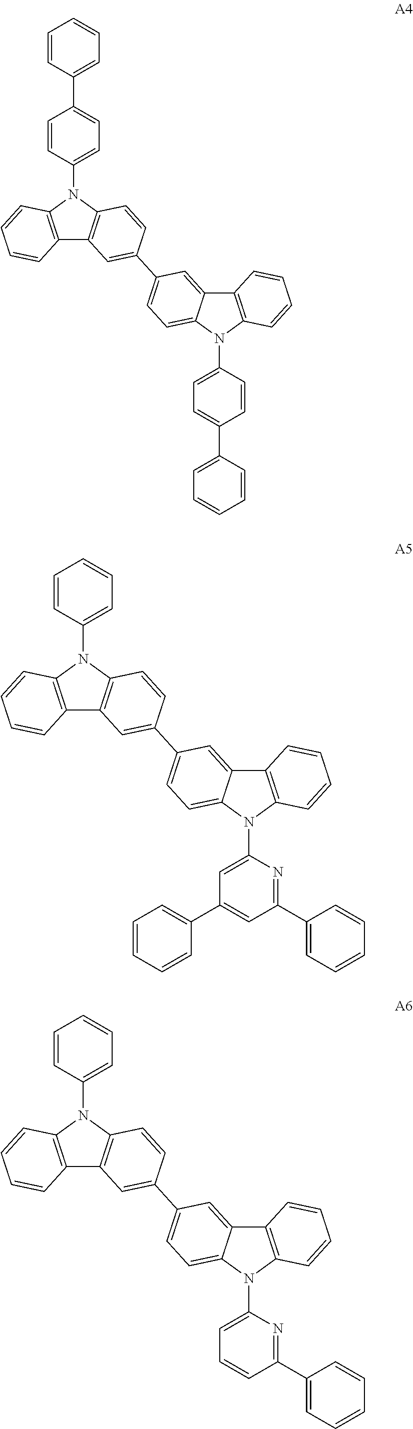 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccccc5)ccc43)n2)cc1]

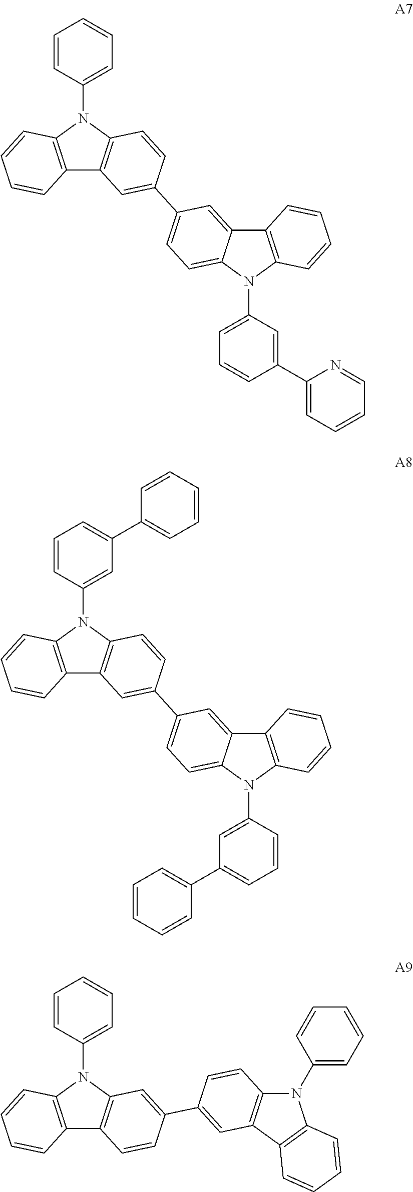 [SMILES: c1ccc(-n2c3ccccc3c3cc(-c4ccc5c(c4)c4ccccc4n5-c4cccc(-c5ccccn5)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c6ccccc6n(-c6ccccc6)c5c4)ccc32)cc1]

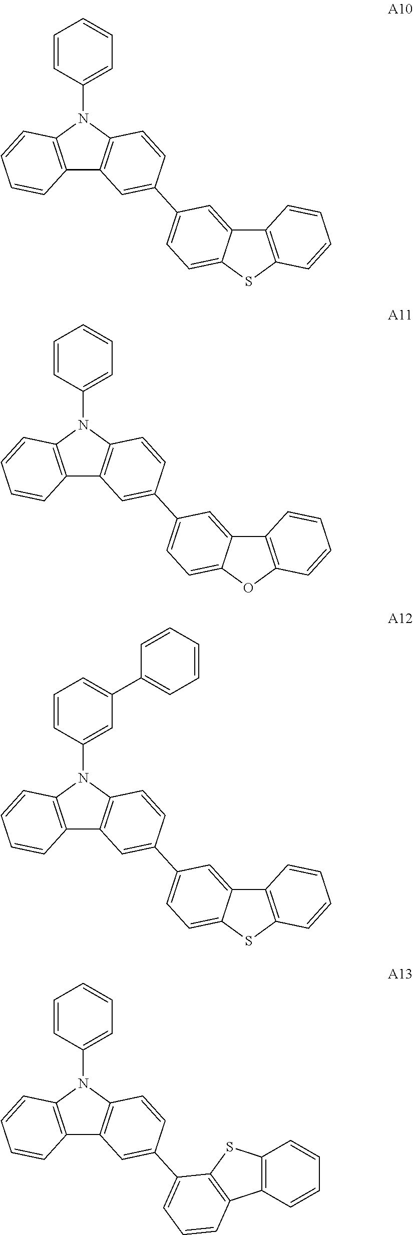 [SMILES: c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1 ; c1ccc(-c2cccc(-n3c4ccccc4c4cc(-c5ccc6sc7ccccc7c6c5)ccc43)c2)cc1 ; c1ccc(-n2c3ccccc3c3cc(-c4cccc5c4sc4ccccc45)ccc32)cc1]

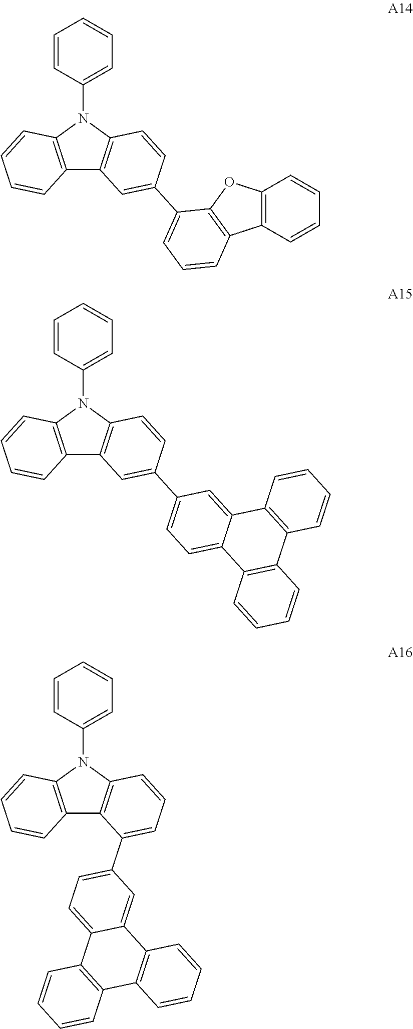 [SMILES: c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1 ; c1ccc(-n2c3ccccc3c3c(-c4ccc5c6ccccc6c6ccccc6c5c4)cccc32)cc1]

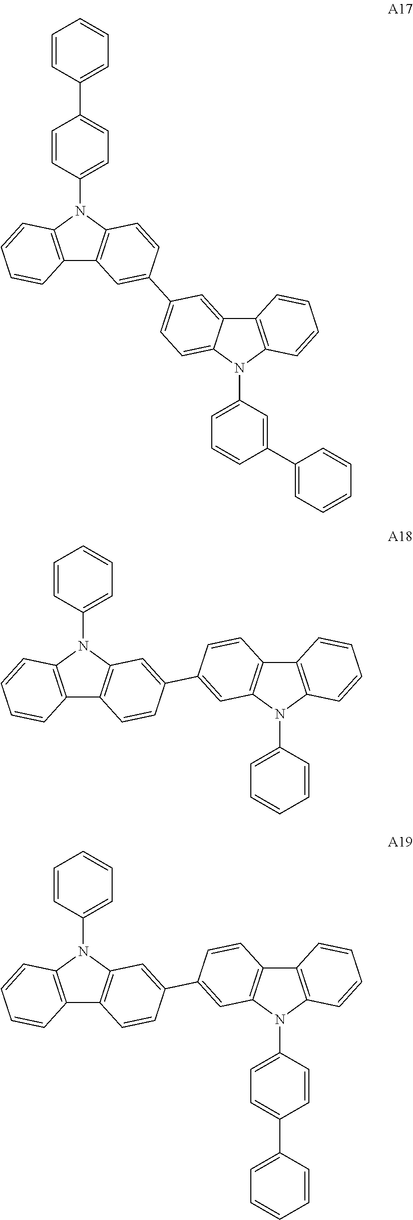 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3ccc(-c4ccc5c6ccccc6n(-c6ccccc6)c5c4)cc32)cc1 ; c1ccc(-c2ccc(-n3c4ccccc4c4ccc(-c5ccc6c7ccccc7n(-c7ccccc7)c6c5)cc43)cc2)cc1]

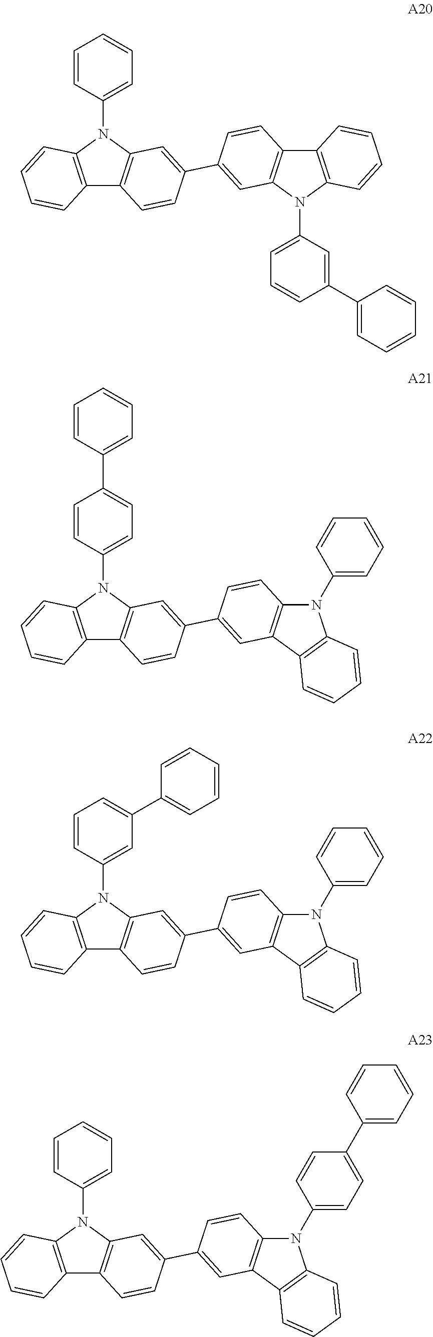 [SMILES: c1ccc(-c2cccc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c(c5)c5ccccc5n6-c5ccccc5)cc43)cc2)cc1 ; c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1]

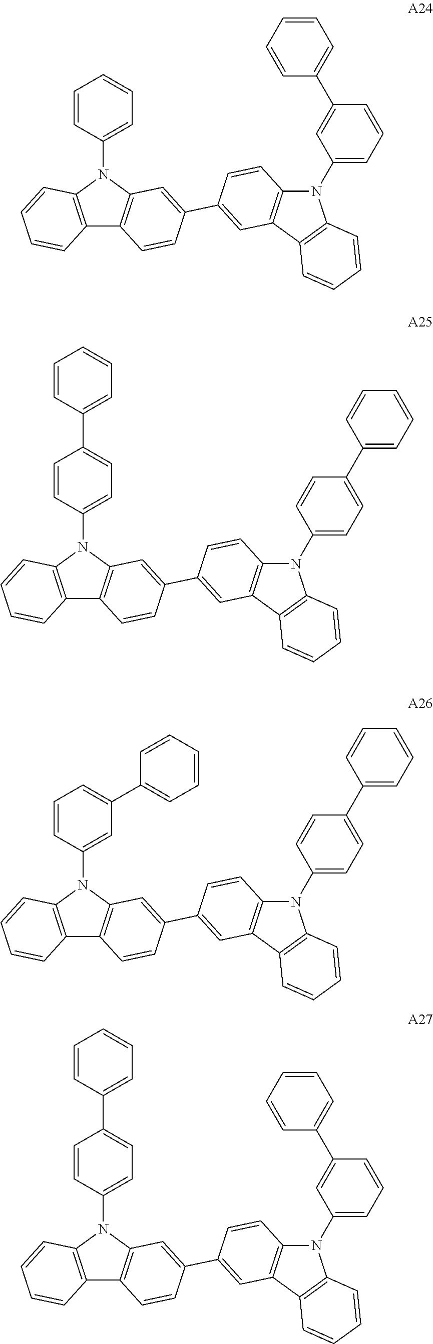 [SMILES: c1ccc(-c2cccc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1]

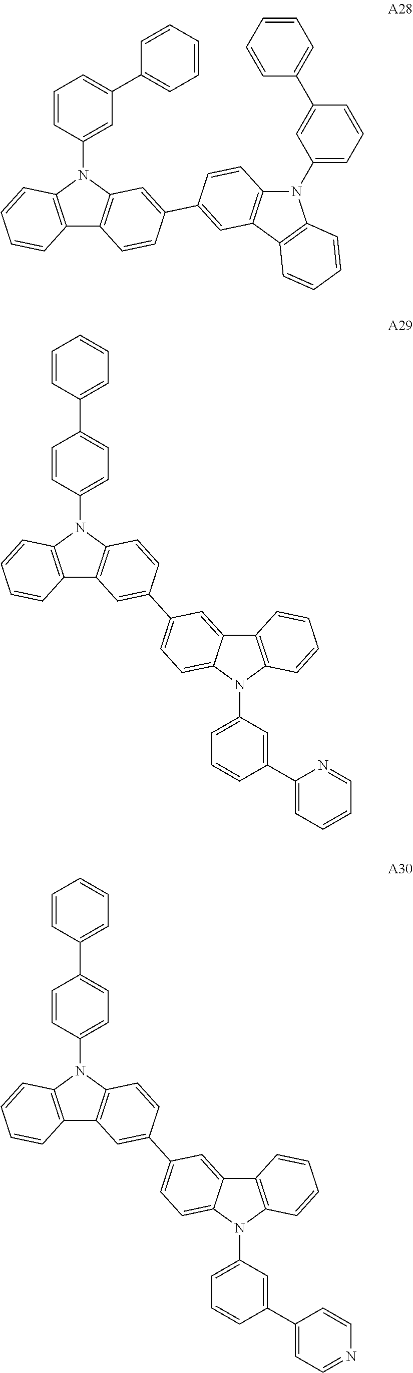 [SMILES: c1ccc(-c2cccc(-n3c4ccccc4c4cc(-c5ccc6c7ccccc7n(-c7cccc(-c8ccccc8)c7)c6c5)ccc43)c2)cc1 ; c1ccc(-c2ccc(-n3c4ccccc4c4cc(-c5ccc6c(c5)c5ccccc5n6-c5cccc(-c6ccccn6)c5)ccc43)cc2)cc1 ; c1ccc(-c2ccc(-n3c4ccccc4c4cc(-c5ccc6c(c5)c5ccccc5n6-c5cccc(-c6ccncc6)c5)ccc43)cc2)cc1]

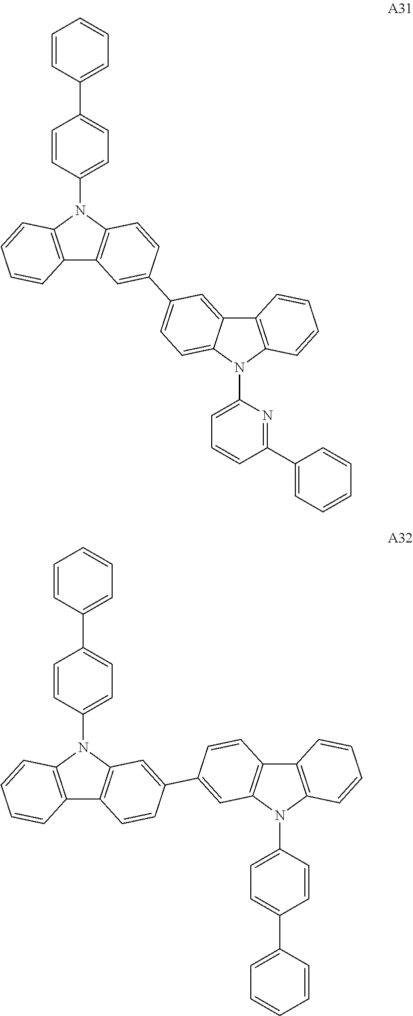 [SMILES: c1ccc(-c2ccc(-n3c4ccccc4c4cc(-c5ccc6c(c5)c5ccccc5n6-c5cccc(-c6ccccc6)n5)ccc43)cc2)cc1 ; c1ccc(-c2ccc(-n3c4ccccc4c4ccc(-c5ccc6c7ccccc7n(-c7ccc(-c8ccccc8)cc7)c6c5)cc43)cc2)cc1]

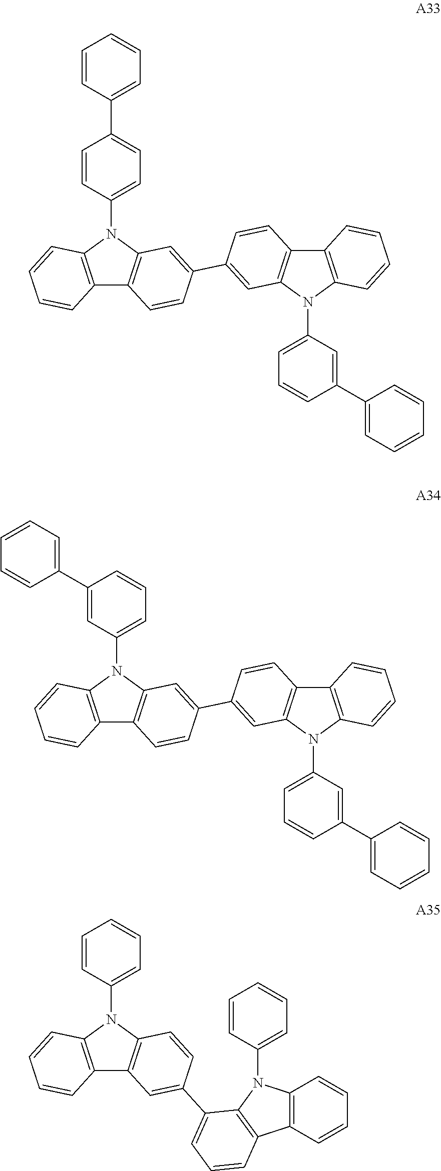 [SMILES: c1ccc(-c2ccc(-n3c4ccccc4c4ccc(-c5ccc6c7ccccc7n(-c7cccc(-c8ccccc8)c7)c6c5)cc43)cc2)cc1 ; c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1]

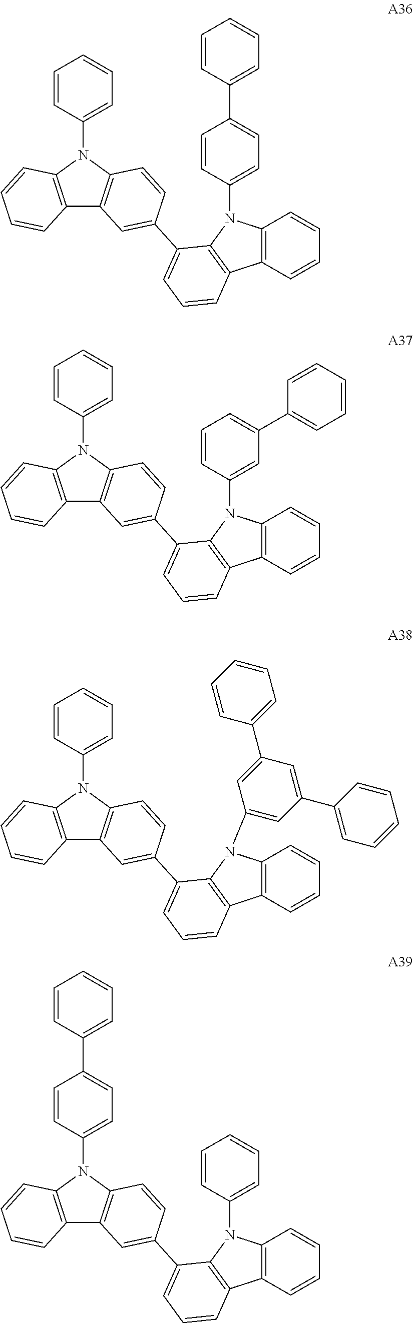 [SMILES: c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1 ; c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1]

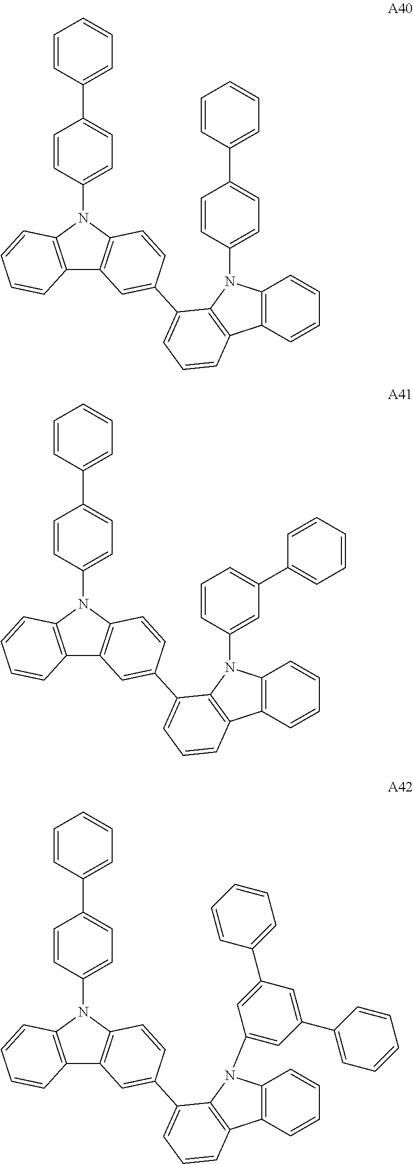 [SMILES: c1ccc(-c2ccc(-n3c4ccccc4c4cc(-c5cccc6c7ccccc7n(-c7ccc(-c8ccccc8)cc7)c56)ccc43)cc2)cc1 ; c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1]

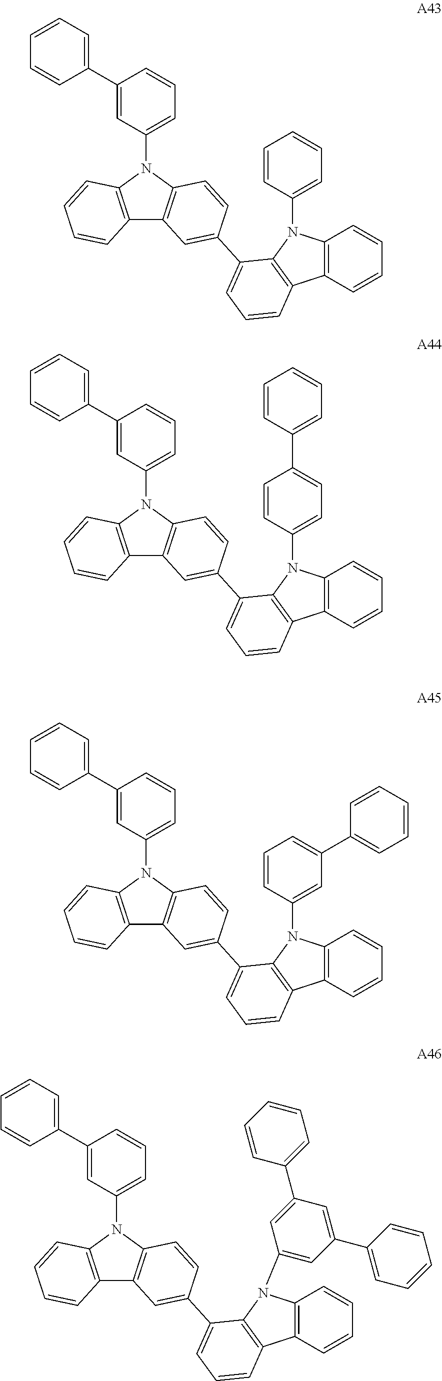 [SMILES: c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1 ; c1ccc(-c2cccc(-n3c4ccccc4c4cc(-c5cccc6c7ccccc7n(-c7cccc(-c8ccccc8)c7)c56)ccc43)c2)cc1 ; c1ccc(-c2cccc(-n3c4ccccc4c4cc(-c5cccc6c7ccccc7n(-c7cc(-c8ccccc8)cc(-c8ccccc8)c7)c56)ccc43)c2)cc1]

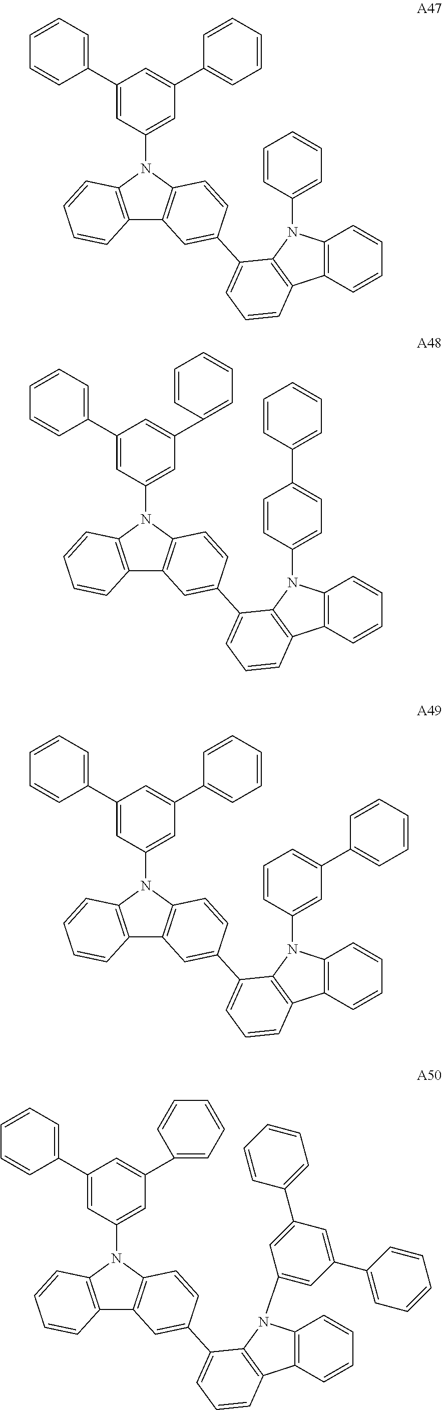 [SMILES: c1ccc(-c2cc(-c3ccccc3)cc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cc(-c6ccccc6)cc(-c6ccccc6)c5)c43)cc2)cc1 ; c1ccc(-c2cc(-c3ccccc3)cc(-n3c4ccccc4c4cc(-c5cccc6c7ccccc7n(-c7cccc(-c8ccccc8)c7)c56)ccc43)c2)cc1 ; c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1]

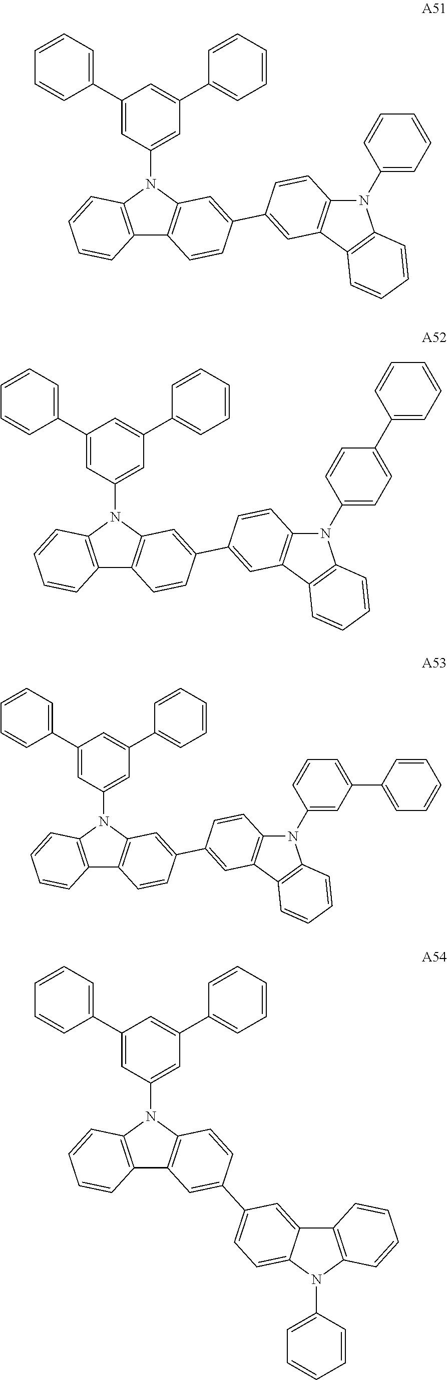 [SMILES: c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

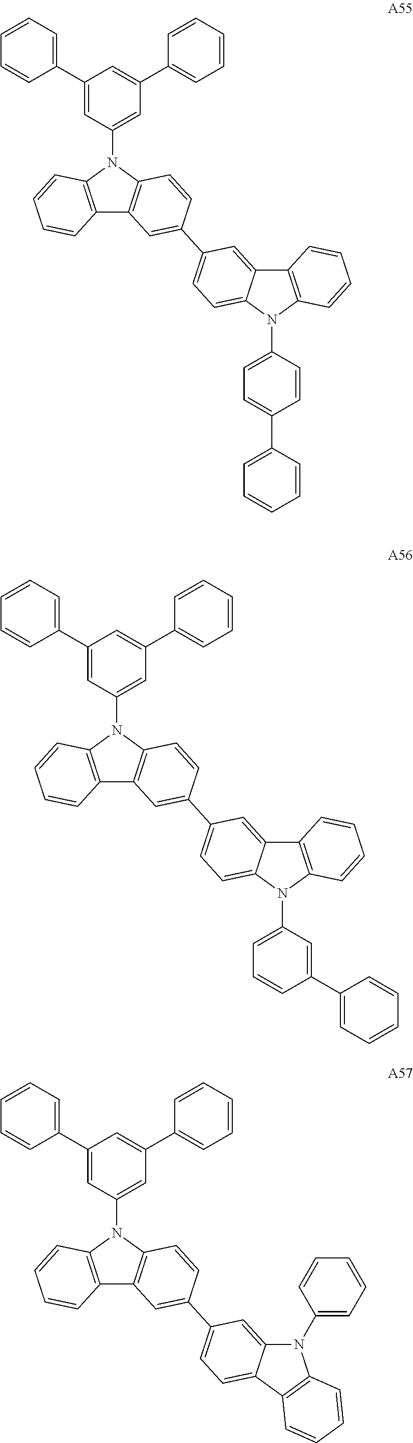 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c7ccccc7n(-c7ccccc7)c6c5)ccc43)c2)cc1]

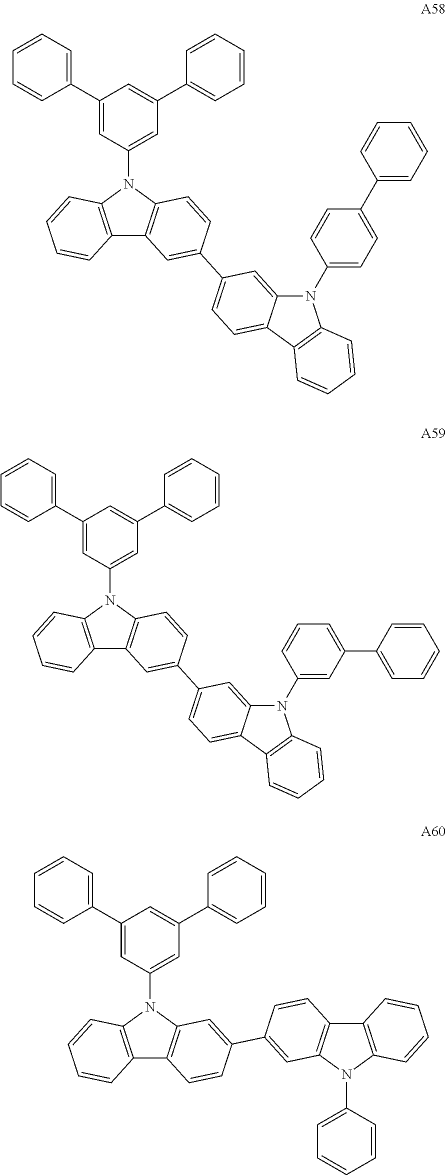 [SMILES: c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1 ; c1ccc(-c2cc(-c3ccccc3)cc(-n3c4ccccc4c4ccc(-c5ccc6c7ccccc7n(-c7ccccc7)c6c5)cc43)c2)cc1]

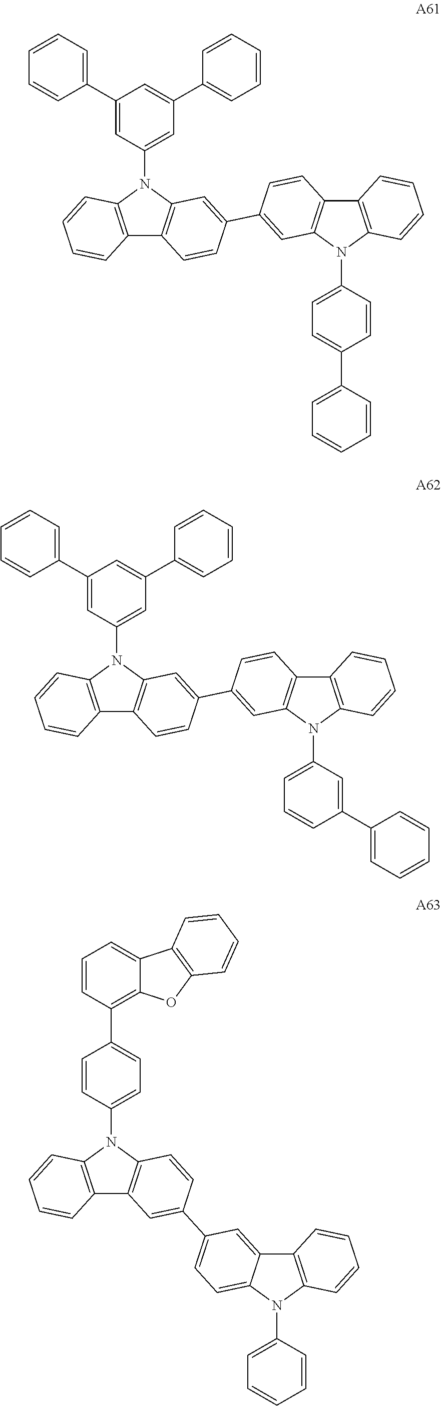 [SMILES: c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1 ; c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-n2c3ccccc3c3cc(-c4ccc5c(c4)c4ccccc4n5-c4ccc(-c5cccc6c5oc5ccccc56)cc4)ccc32)cc1]

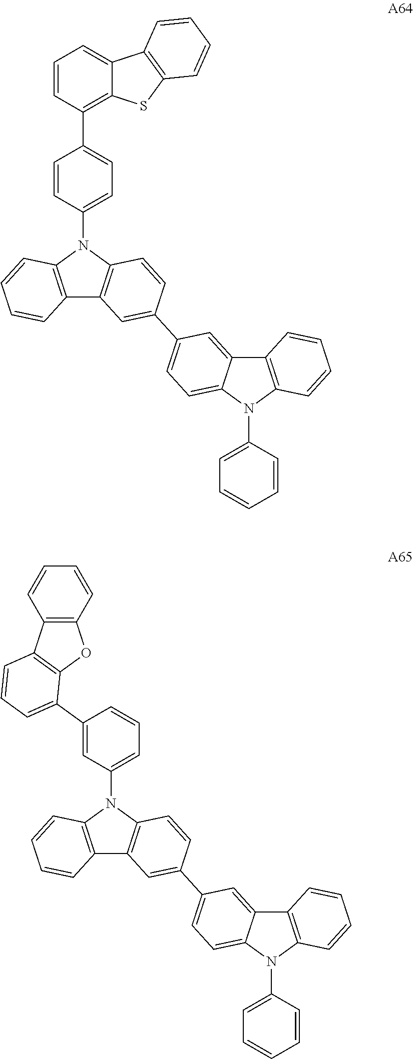 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4cccc(-c5cccc6c5oc5ccccc56)c4)ccc32)cc1]

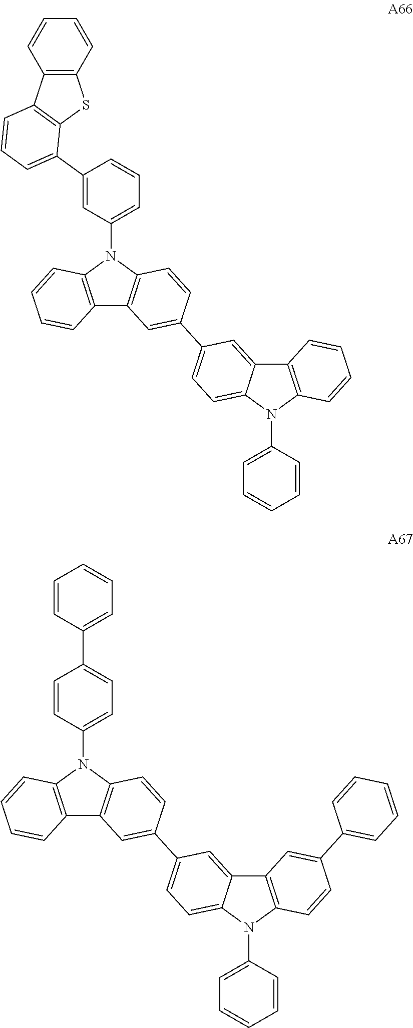 [SMILES: c1ccc(-n2c3ccccc3c3cc(-c4ccc5c(c4)c4ccccc4n5-c4cccc(-c5cccc6c5sc5ccccc56)c4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

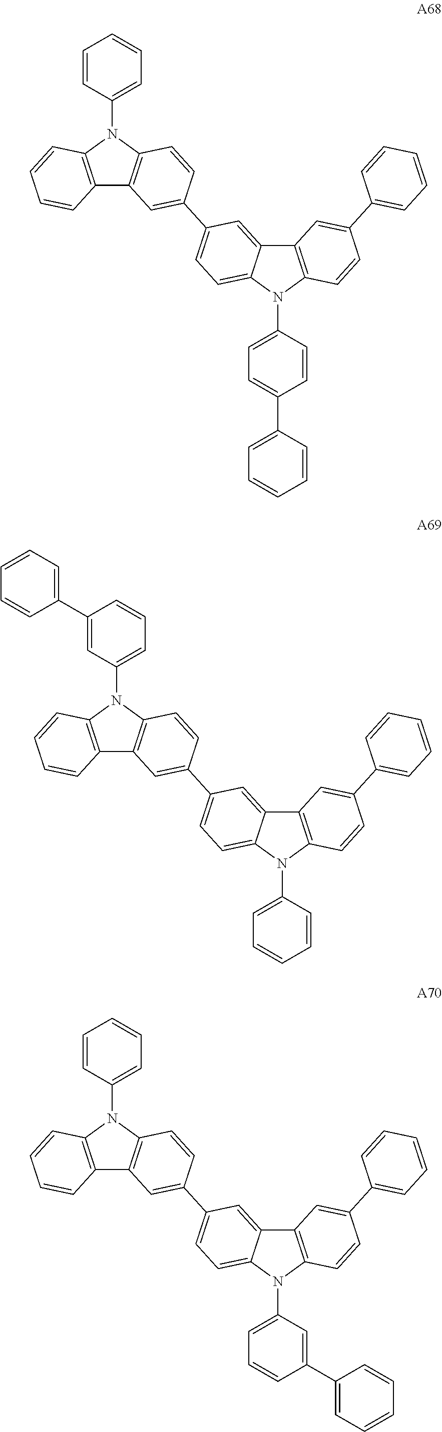 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

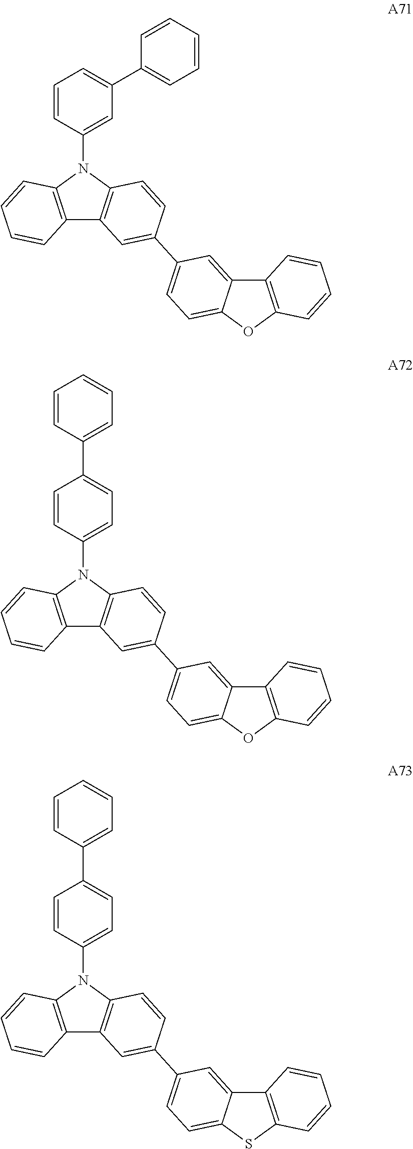 [SMILES: c1ccc(-c2cccc(-n3c4ccccc4c4cc(-c5ccc6oc7ccccc7c6c5)ccc43)c2)cc1 ; c1ccc(-c2ccc(-n3c4ccccc4c4cc(-c5ccc6oc7ccccc7c6c5)ccc43)cc2)cc1 ; c1ccc(-c2ccc(-n3c4ccccc4c4cc(-c5ccc6sc7ccccc7c6c5)ccc43)cc2)cc1]

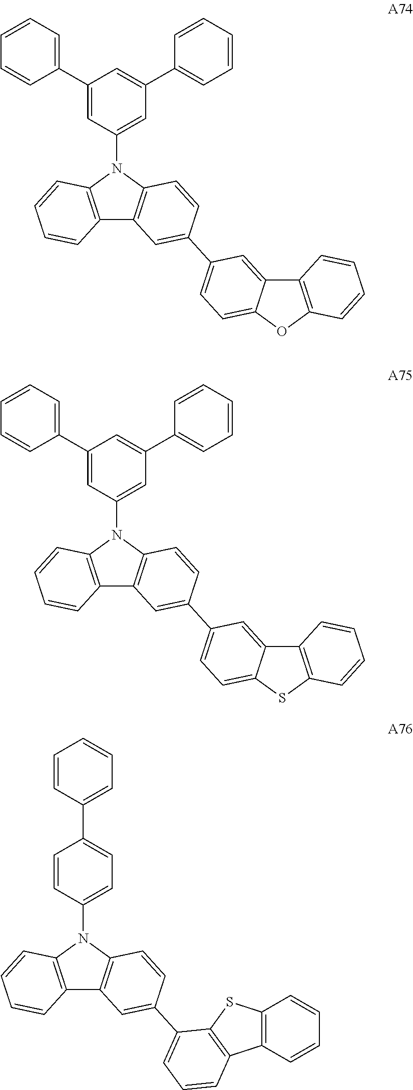 [SMILES: c1ccc(-c2cc(-c3ccccc3)cc(-n3c4ccccc4c4cc(-c5ccc6oc7ccccc7c6c5)ccc43)c2)cc1 ; c1ccc(-c2cc(-c3ccccc3)cc(-n3c4ccccc4c4cc(-c5ccc6sc7ccccc7c6c5)ccc43)c2)cc1 ; c1ccc(-c2ccc(-n3c4ccccc4c4cc(-c5cccc6c5sc5ccccc56)ccc43)cc2)cc1]

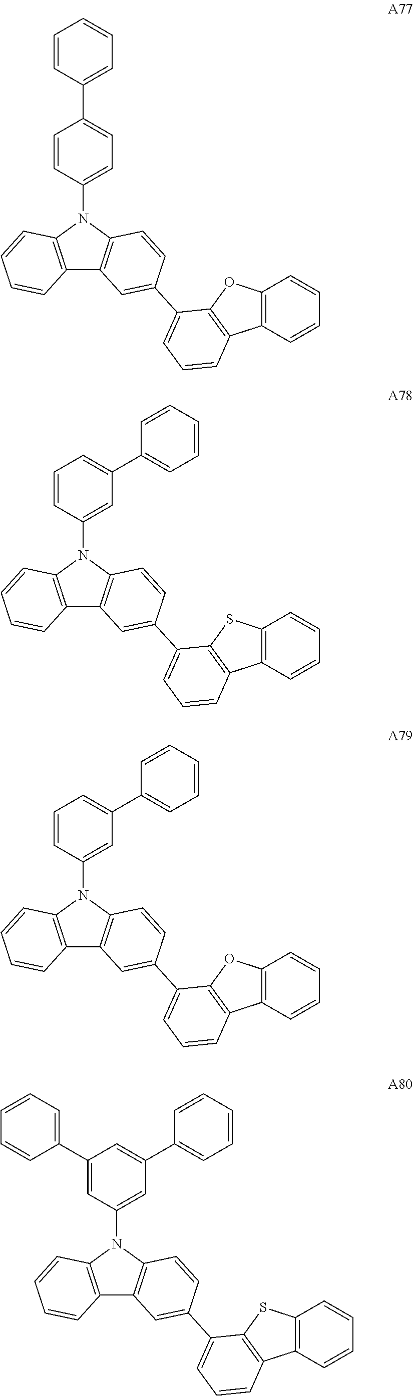

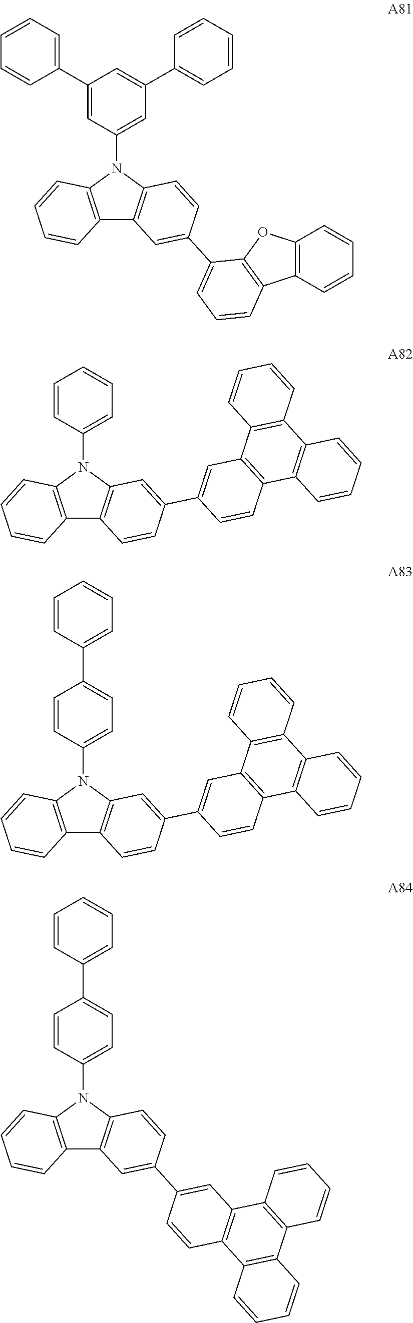 [SMILES: c1ccc(-c2cc(-c3ccccc3)cc(-n3c4ccccc4c4cc(-c5cccc6c5oc5ccccc56)ccc43)c2)cc1 ; c1ccc(-n2c3ccccc3c3ccc(-c4ccc5c6ccccc6c6ccccc6c5c4)cc32)cc1 ; c1ccc(-c2ccc(-n3c4ccccc4c4ccc(-c5ccc6c7ccccc7c7ccccc7c6c5)cc43)cc2)cc1 ; c1ccc(-c2ccc(-n3c4ccccc4c4cc(-c5ccc6c7ccccc7c7ccccc7c6c5)ccc43)cc2)cc1]

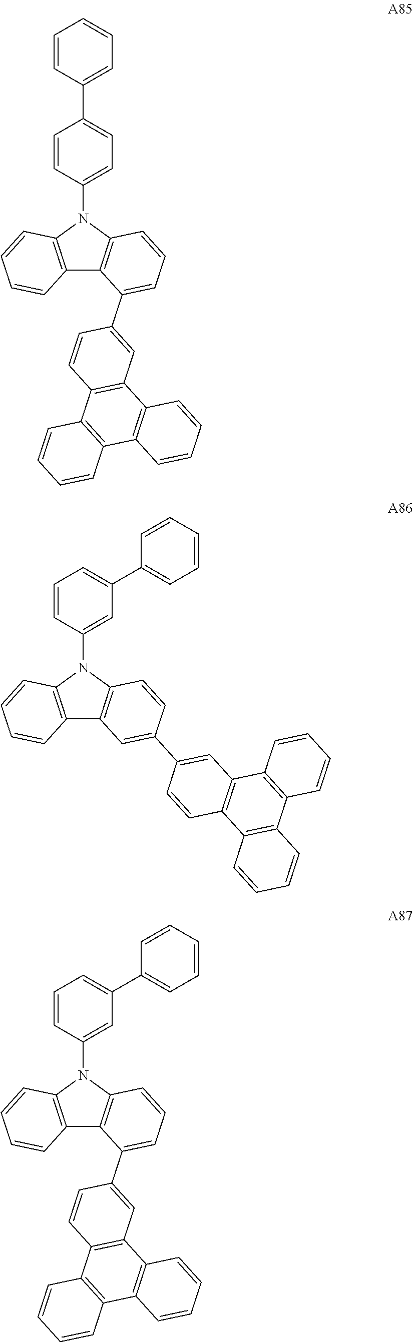 [SMILES: c1ccc(-c2ccc(-n3c4ccccc4c4c(-c5ccc6c7ccccc7c7ccccc7c6c5)cccc43)cc2)cc1 ; c1ccc(-c2cccc(-n3c4ccccc4c4cc(-c5ccc6c7ccccc7c7ccccc7c6c5)ccc43)c2)cc1 ; c1ccc(-c2cccc(-n3c4ccccc4c4c(-c5ccc6c7ccccc7c7ccccc7c6c5)cccc43)c2)cc1]

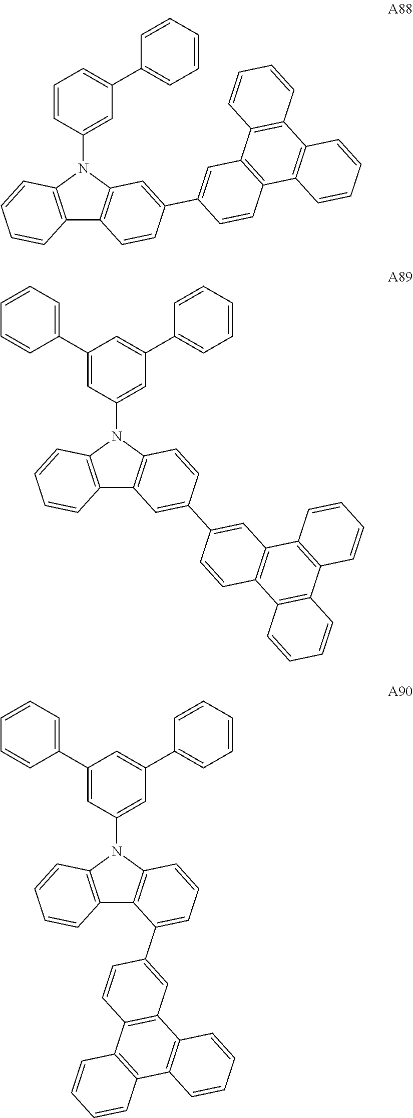

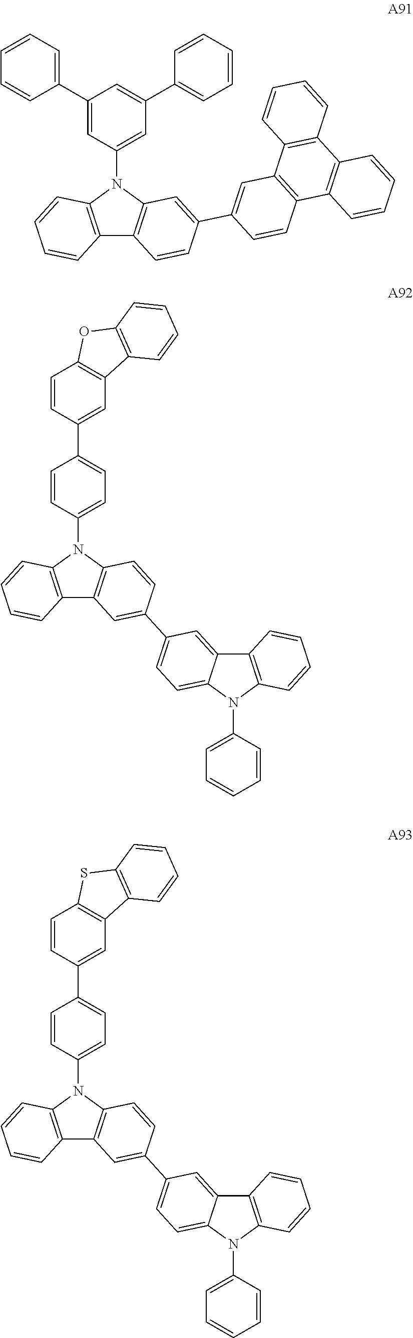 [SMILES: c1ccc(-c2cc(-c3ccccc3)cc(-n3c4ccccc4c4ccc(-c5ccc6c7ccccc7c7ccccc7c6c5)cc43)c2)cc1 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

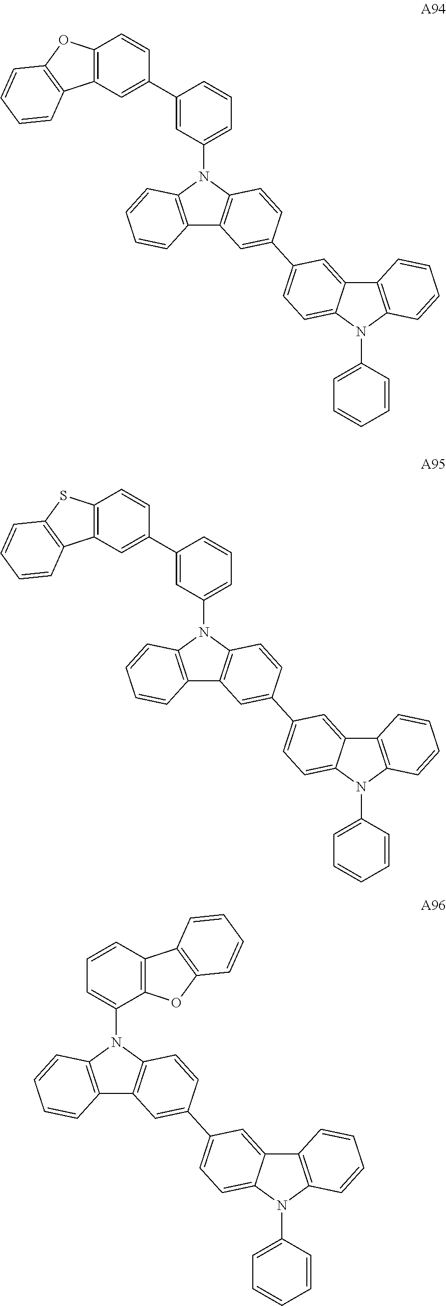 [SMILES: c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4cccc5c4oc4ccccc45)ccc32)cc1]

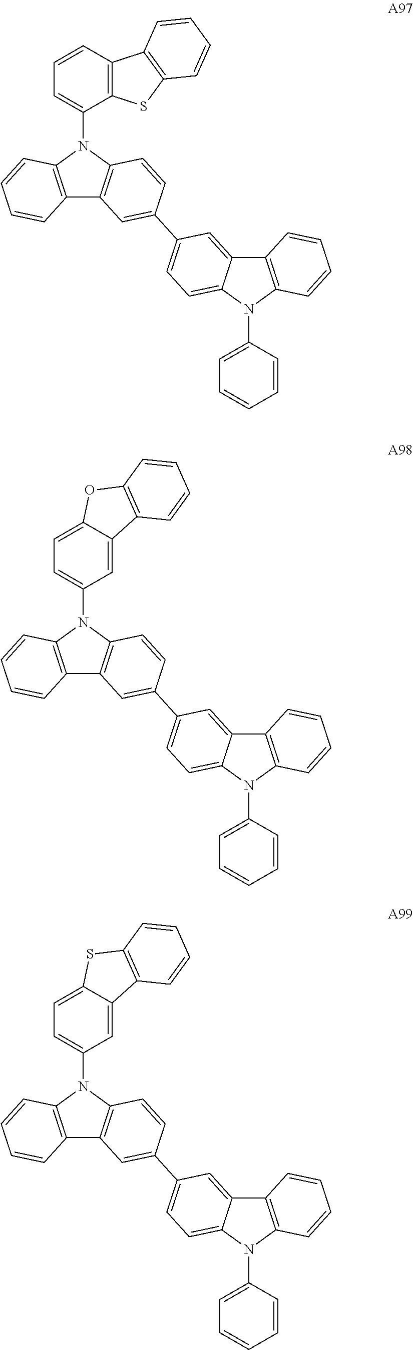 [SMILES: c1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

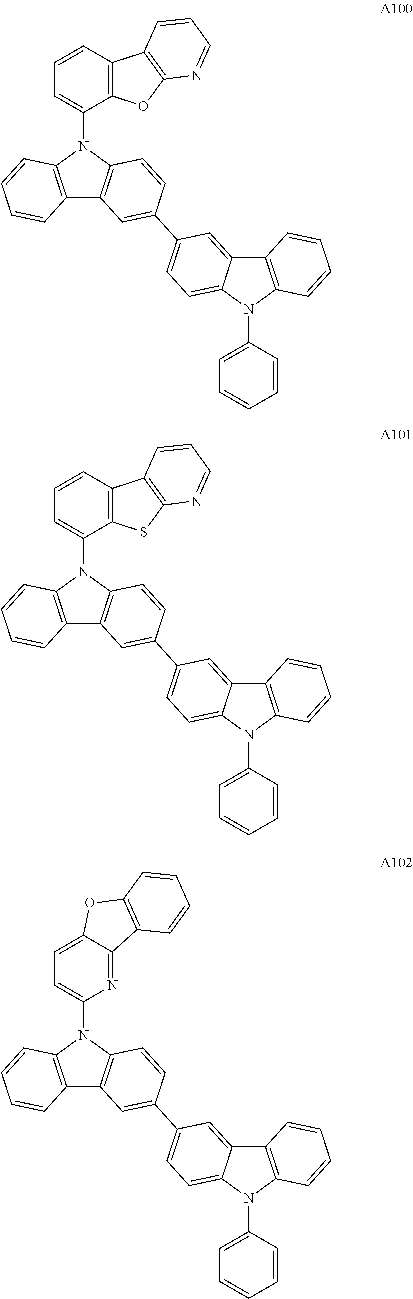 [SMILES: c1ccc(-n2c3ccccc3c3cc(-c4ccc5c(c4)c4ccccc4n5-c4cccc5c4oc4ncccc45)ccc32)cc1 ; c1ccc(-n2c3ccccc3c3cc(-c4ccc5c(c4)c4ccccc4n5-c4cccc5c4sc4ncccc45)ccc32)cc1 ; c1ccc(-n2c3ccccc3c3cc(-c4ccc5c(c4)c4ccccc4n5-c4ccc5oc6ccccc6c5n4)ccc32)cc1]

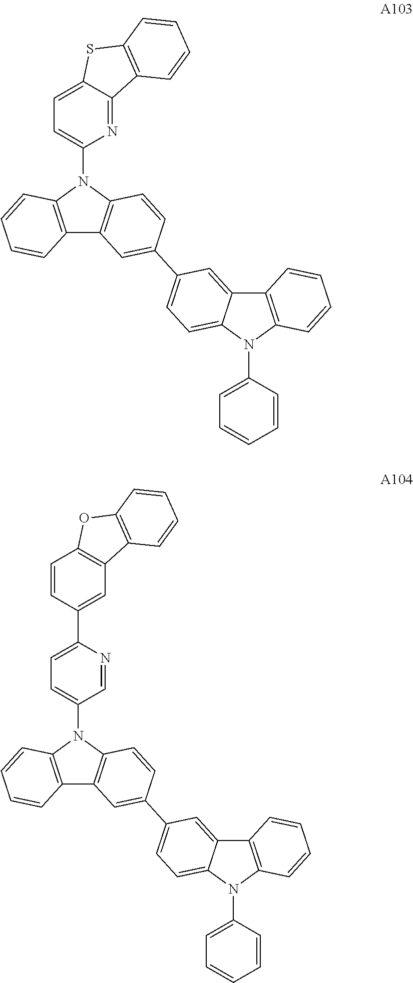 [SMILES: c1ccc(-n2c3ccccc3c3cc(-c4ccc5c(c4)c4ccccc4n5-c4ccc5sc6ccccc6c5n4)ccc32)cc1 ; c1ccc(-n2c3ccccc3c3cc(-c4ccc5c(c4)c4ccccc4n5-c4ccc(-c5ccc6oc7ccccc7c6c5)nc4)ccc32)cc1]

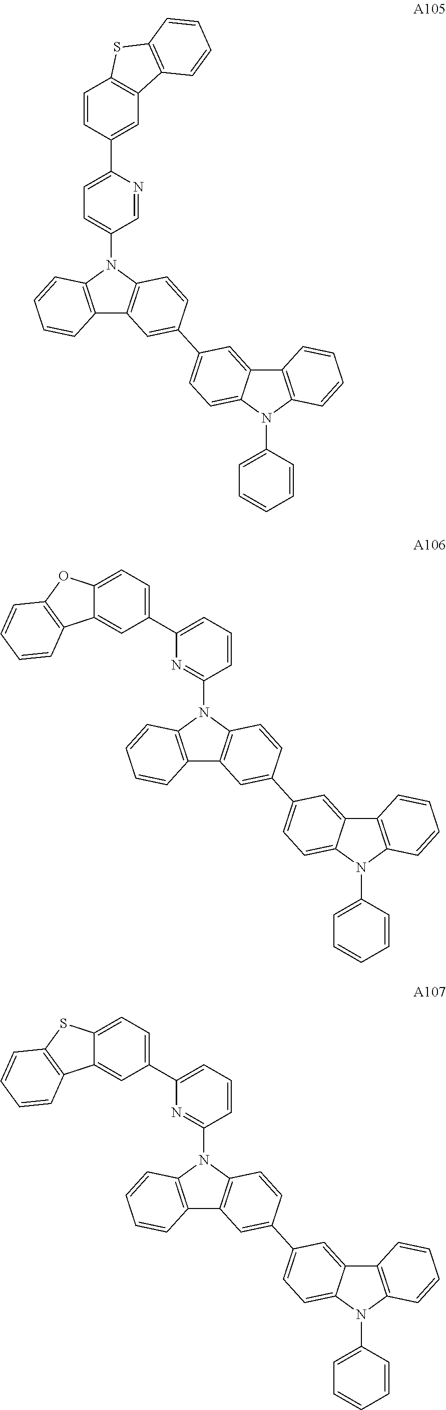 [SMILES: c1ccc(-n2c3ccccc3c3cc(-c4ccc5c(c4)c4ccccc4n5-c4ccc(-c5ccc6sc7ccccc7c6c5)nc4)ccc32)cc1 ; c1ccc(-n2c3ccccc3c3cc(-c4ccc5c(c4)c4ccccc4n5-c4cccc(-c5ccc6oc7ccccc7c6c5)n4)ccc32)cc1 ; c1ccc(-n2c3ccccc3c3cc(-c4ccc5c(c4)c4ccccc4n5-c4cccc(-c5ccc6sc7ccccc7c6c5)n4)ccc32)cc1]

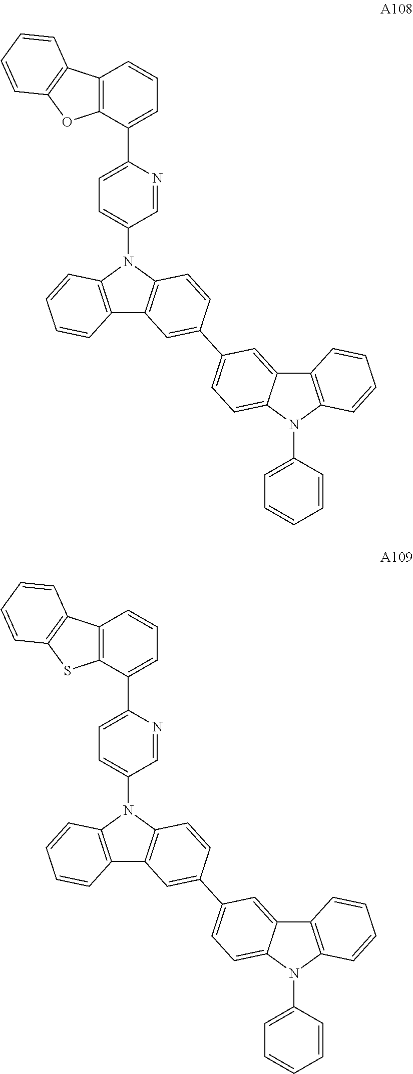 [SMILES: c1ccc(-n2c3ccccc3c3cc(-c4ccc5c(c4)c4ccccc4n5-c4ccc(-c5cccc6c5oc5ccccc56)nc4)ccc32)cc1 ; c1ccc(-n2c3ccccc3c3cc(-c4ccc5c(c4)c4ccccc4n5-c4ccc(-c5cccc6c5sc5ccccc56)nc4)ccc32)cc1]

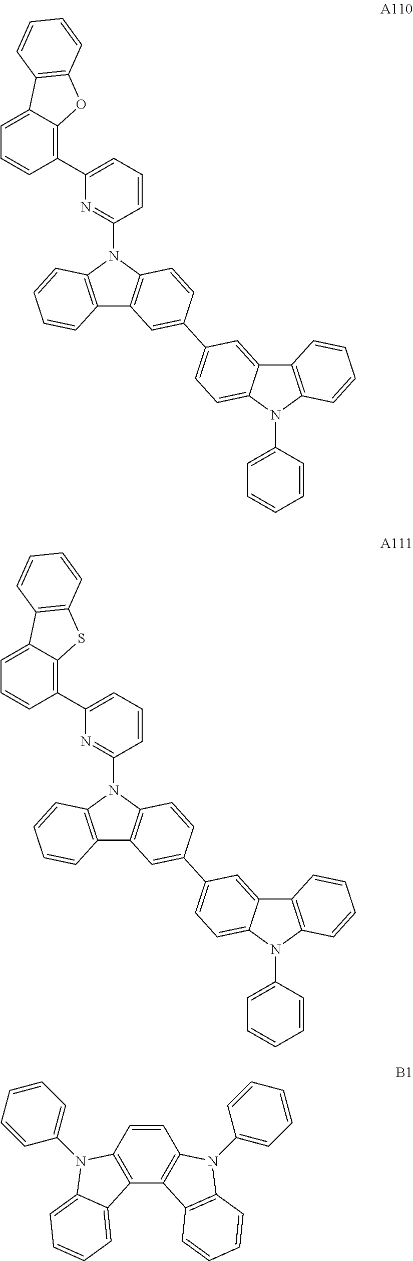

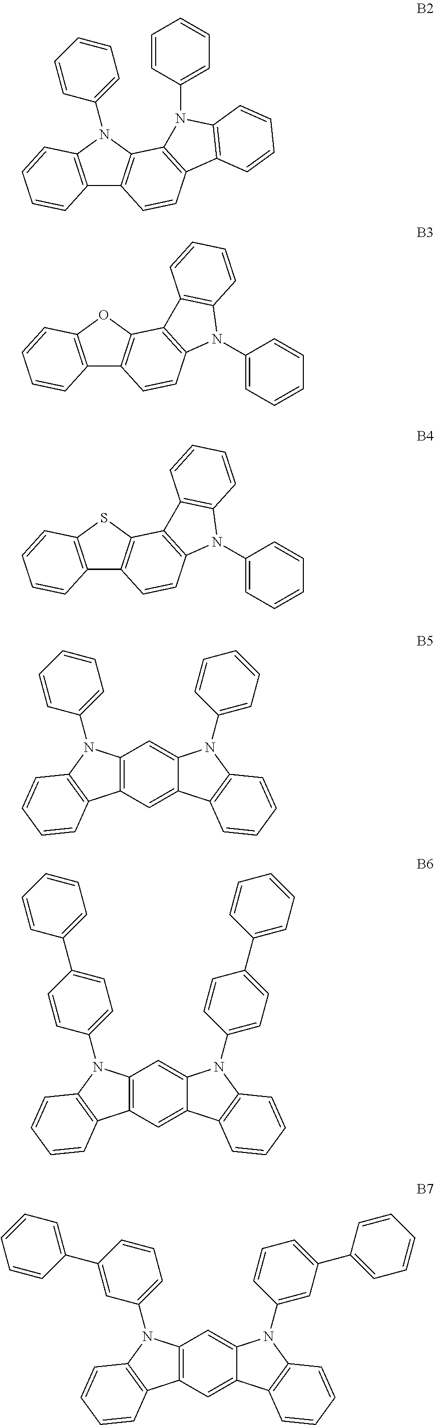

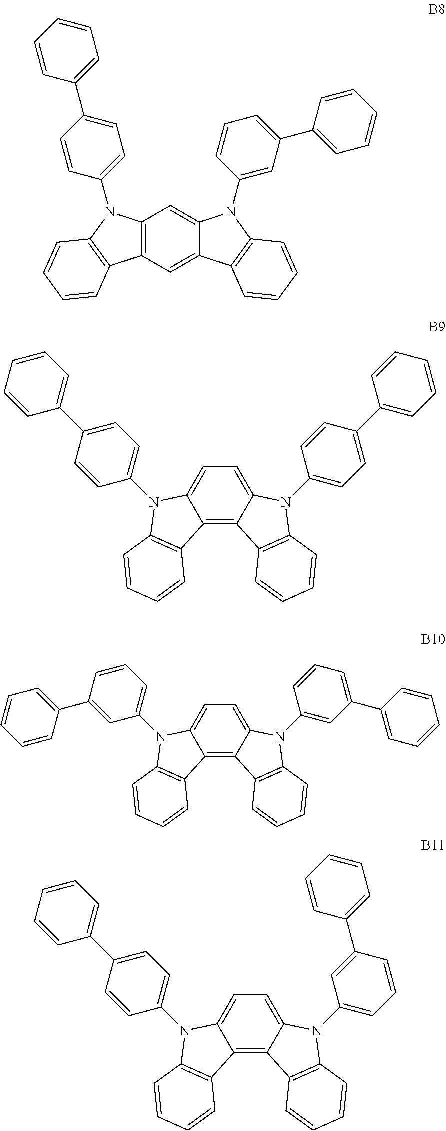 [SMILES: c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2ccc(-n3c4ccccc4c4c5c6ccccc6n(-c6cccc(-c7ccccc7)c6)c5ccc43)cc2)cc1]

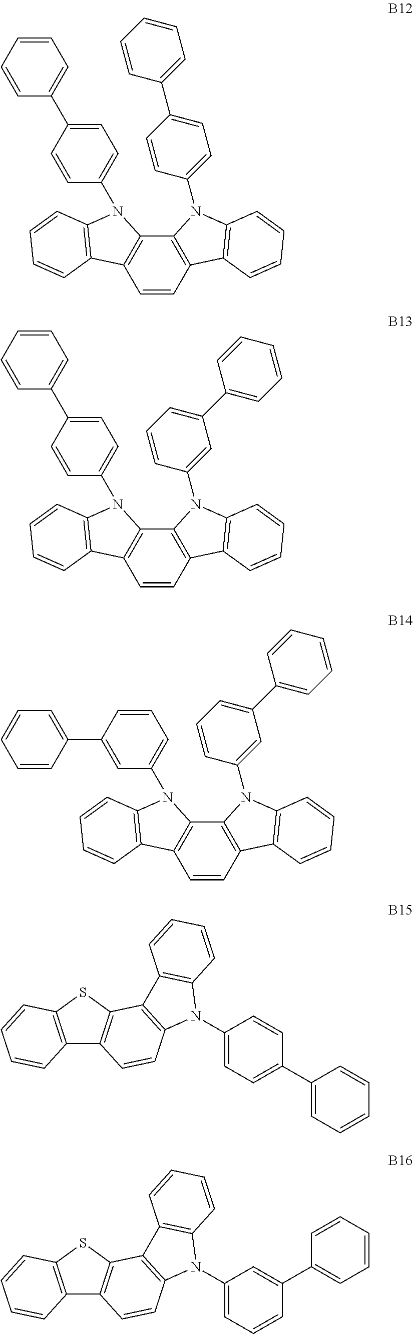

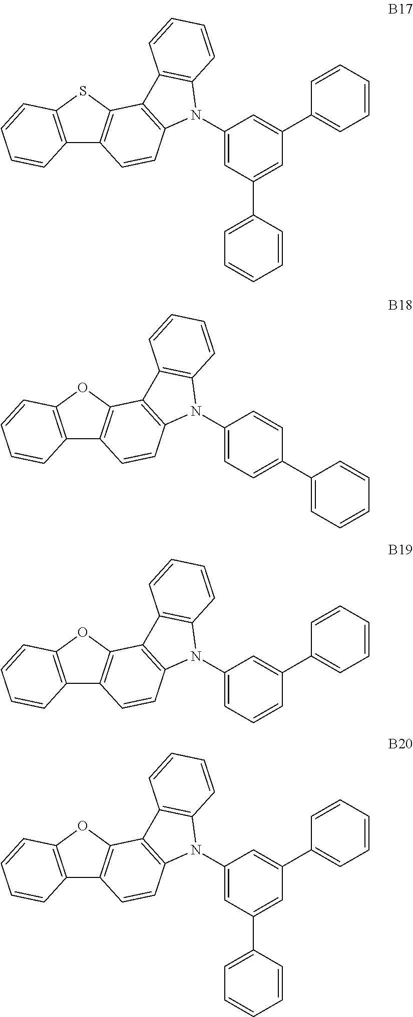 [SMILES: c1ccc(-c2cc(-c3ccccc3)cc(-n3c4ccccc4c4c5sc6ccccc6c5ccc43)c2)cc1 ; c1ccc(-c2ccc(-n3c4ccccc4c4c5oc6ccccc6c5ccc43)cc2)cc1 ; c1ccc(-c2cccc(-n3c4ccccc4c4c5oc6ccccc6c5ccc43)c2)cc1 ; c1ccc(-c2cc(-c3ccccc3)cc(-n3c4ccccc4c4c5oc6ccccc6c5ccc43)c2)cc1]

For example, a weight ratio of the first host to the second host may be in a range of about 1:99 to about 99:1, and in some embodiments, about 10:90 to about 90:10. When the weight ratio of the first host to the second host is within these ranges, the electron transport characteristics of the first host and the hole transport characteristics of the second host may reach equilibrium, so that the emission efficiency and lifetime of the organic light-emitting device may be improved.

When the EML includes both a host and a dopant, the amount of the dopant may be from about 0.01 to about 15 parts by weight based on 100 parts by weight of the host. However, the amount of the dopant is not limited to this range.

Synthesis methods of the condensed cyclic compound of Formula 1 above, the first compound of Formula 41 above, and the second compound of Formula 61 above may be easily understood to one of ordinary skill in the art based on the synthesis examples described below.

When the organic light-emitting device is a full color organic light-emitting device, the emission layer may be patterned into a red emission layer, a green emission layer, and a blue emission layer. In some embodiments, the EML may have a stack structure including a red emission layer, a green emission layer, and/or a blue emission layer that are stacked upon one another to emit white light, but is not limited thereto. A host of one of the red emission layer, the green emission layer, and the blue emission layer may include the condensed cyclic compound of Formula 1 above. For example, the host of the green emission layer may include the condensed cyclic compound of Formula 1.

In addition, the electron transport auxiliary layer on the blue emission layer may include the condensed cyclic compound represented by Formula 1.

The EML of the light-emitting device may include a dopant, which may be a fluorescent dopant emitting light based on fluorescence mechanism, or a phosphorescent dopant emitting light based on phosphorescence mechanism.

In some embodiments, the EML may include a host including at least one of the condensed cyclic compound of Formula 1, and a phosphorescent dopant. The phosphorescent dopant may include an organometallic complex including a transition metal, for example, iridium (Ir), platinum (Pt), osmium (Os), or rhodium (Rh).

The phosphorescent dopant may include an organometallic compound represented by Formula 81 below:

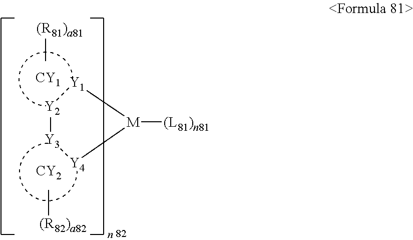

In Formula 81,

M may be iridium (Ir), platinum (Pt), osmium (Os), titanium (Ti), zirconium (Zr), hafnium (Hf), europium (Eu), terbium (Tb), or thulium (Tm);

Y₁ to Y₄ may be each independently a carbon (C) or a nitrogen (N);

Y₁ and Y₂ may be linked to each other via a single bond or a double bond, and Y₃ and Y₄ may be linked to each other via a single bond or a double bond;

CY₁ and CY₂ may be each independently benzene, naphthalene, fluorene, spiro-fluorene, indene, pyrrole, thiophene, furan, imidazole, pyrazole, thiazole, isothiazole, oxazole, isooxazole, pyridine, pyrazine, pyrimidine, pyridazine, quinoline, isoquinoline, benzoquinoline, quinoxaline, quinazoline, carbazole, benzoimidazole, benzofuran (benzofuran), benzothiophene, isobenzothiophene, benzooxazole, isobenzooxazole, triazole, tetrazole, oxadiazole, triazine, dibenzofuran, or dibenzothiophene, wherein CY₁ and CY₂ may be optionally linked to each other via a single bond or an organic linking group;

R₈₁ and R₈₂ may be each independently selected from a hydrogen, a deuterium, —F, —Cl, —Br, —I, a hydroxyl group, a cyano group, a nitro group, an amino group, an amidino group, a hydrazine group, a hydrazone, a carboxylic acid group or a salt thereof, a sulfonic acid group or a salt thereof, a phosphoric acid group or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), and —B(Q₆)(Q₇);

a81 and a82 may be each independently an integer selected from 1 to 5;

n81 may be an integer selected from 0 to 4;

n82 may be 1, 2, or 3;

L₈₁ may be selected from a monovalent organic ligand, a divalent organic ligand, and a trivalent organic ligand.

R₈₁ and R₈₂ in Formula 81 may be defined to be the same as described above with reference to R₁₁ above.

The phosphorescent dopant may include at least one of Compounds PD1 to PD78, but is not limited thereto (the following Compound PD1 is Ir(ppy)₃):

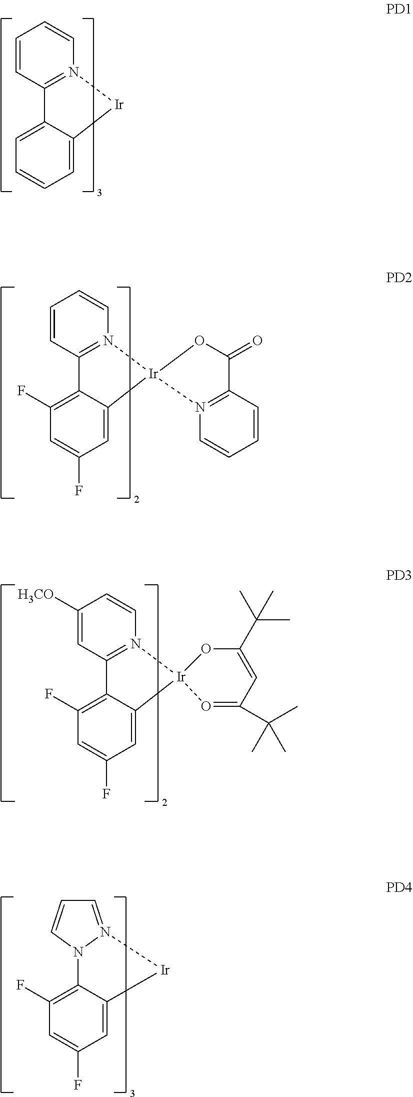

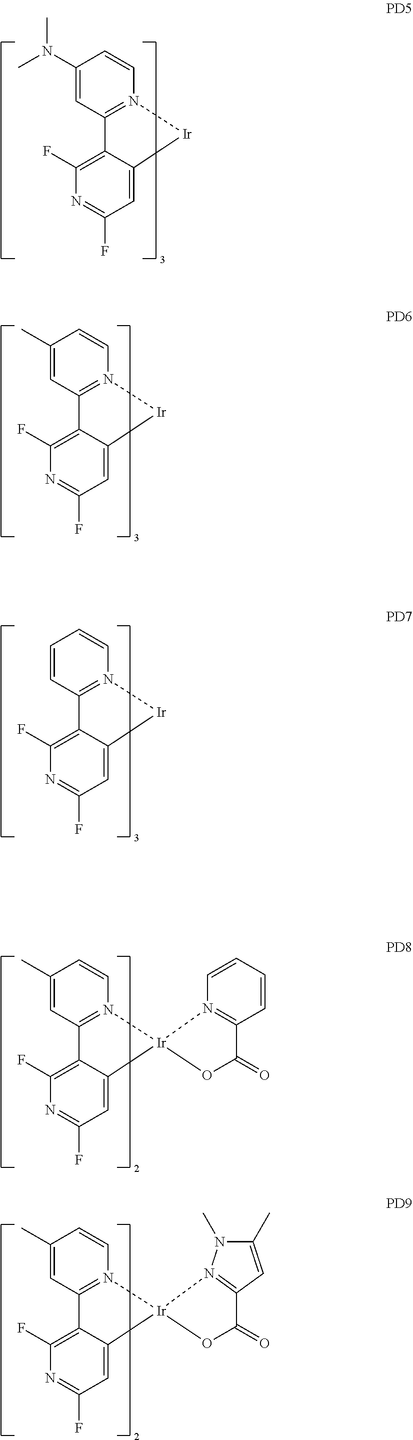

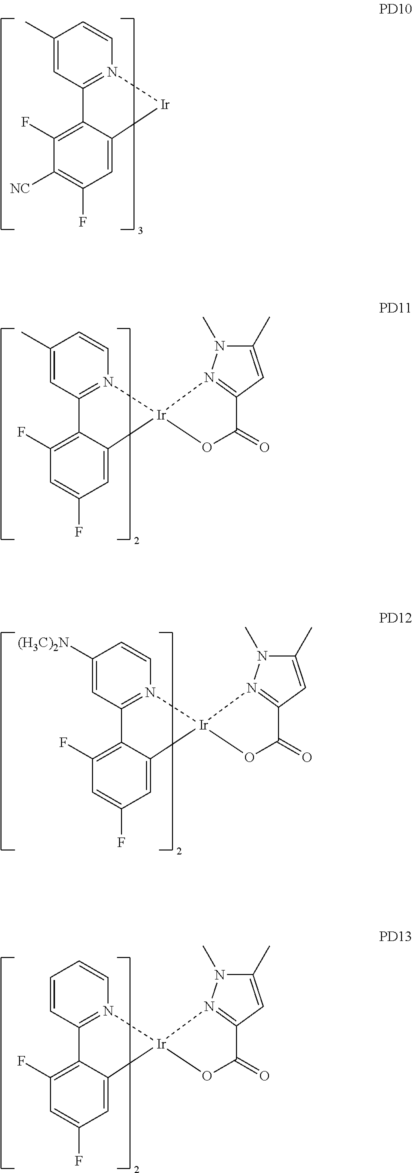

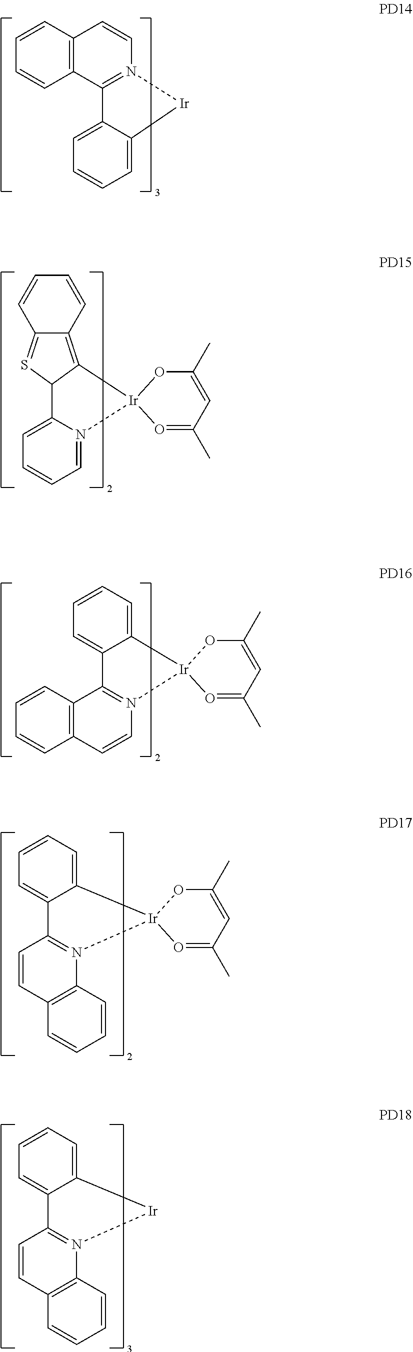

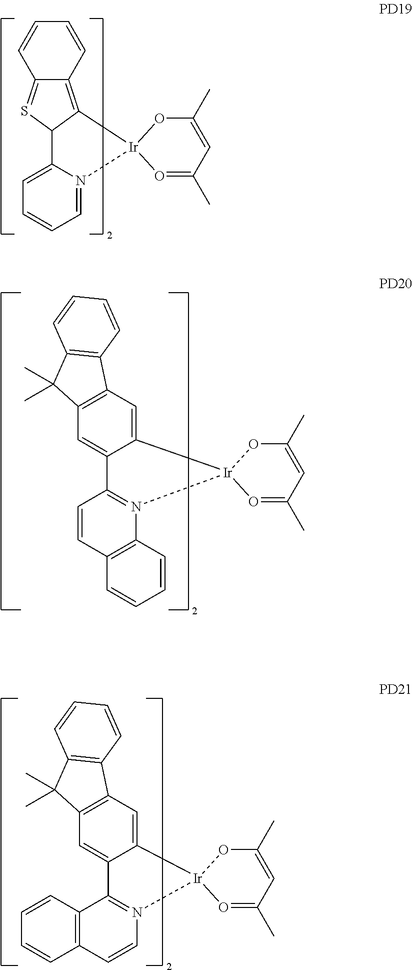

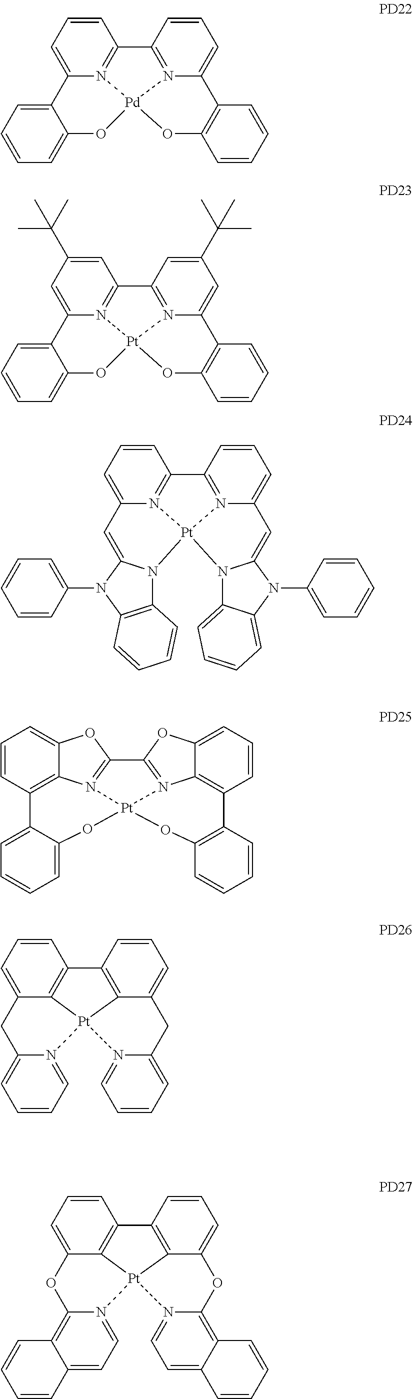

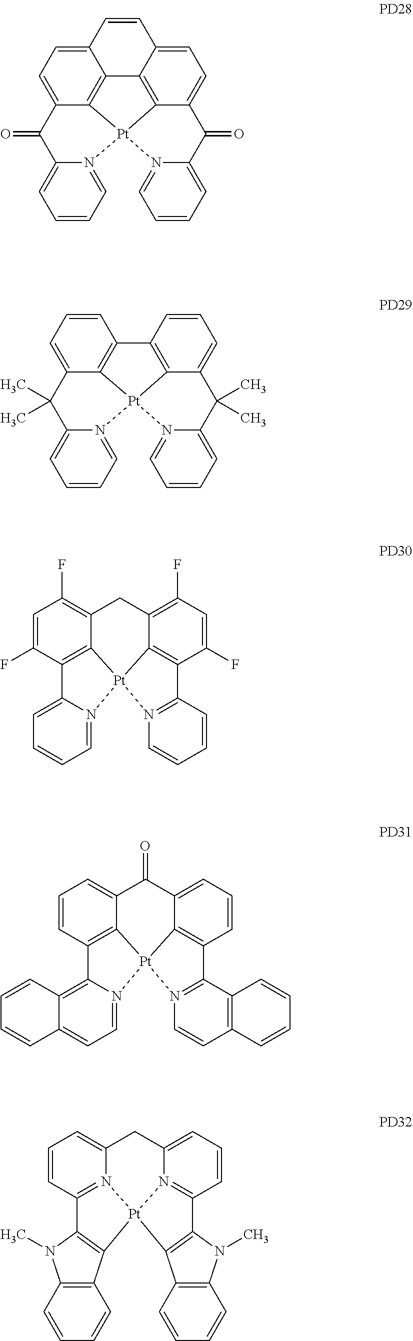

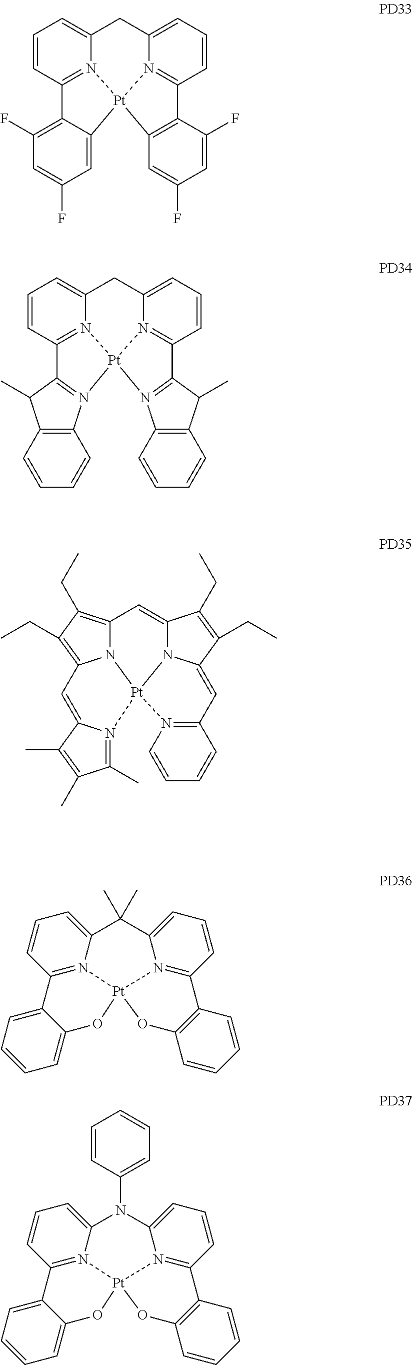

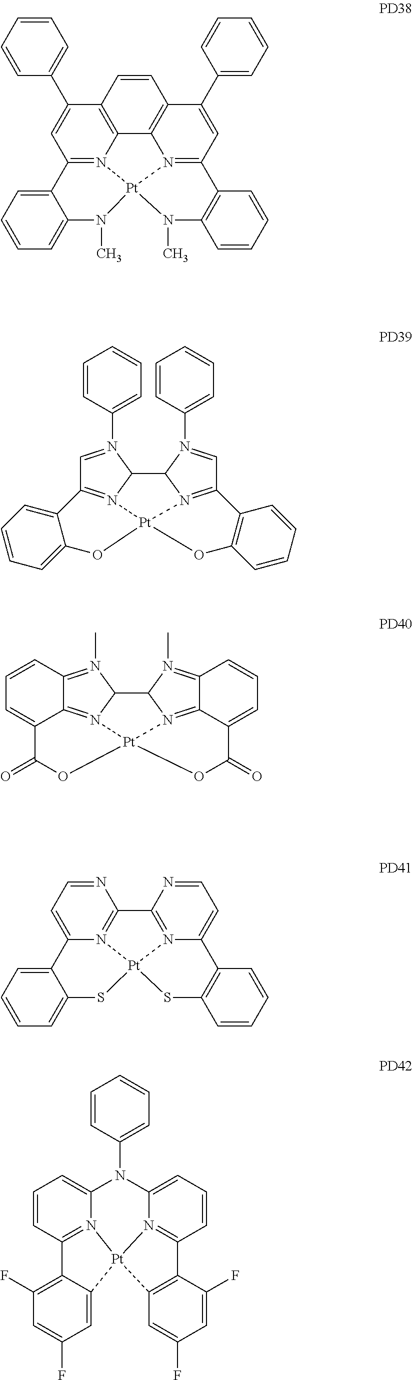

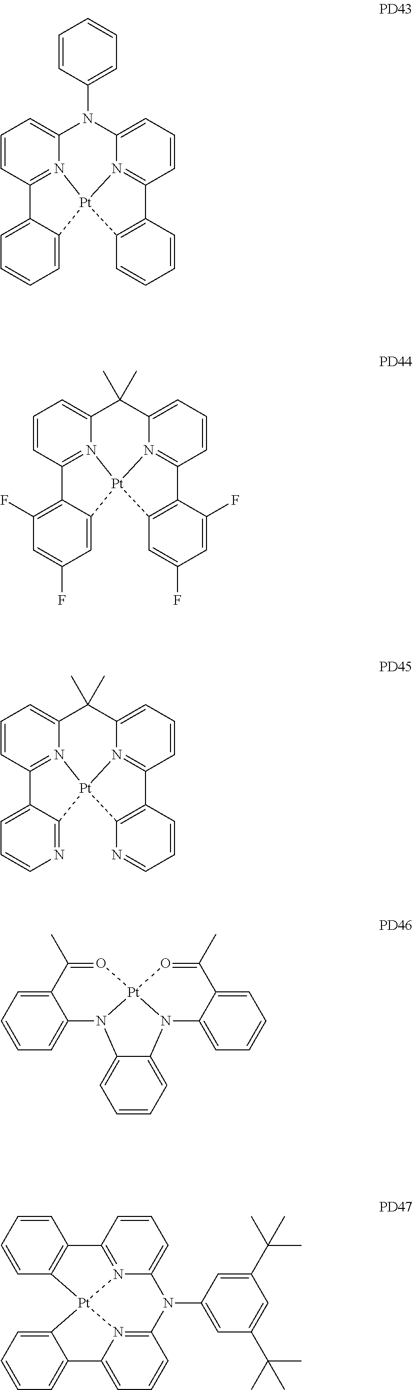

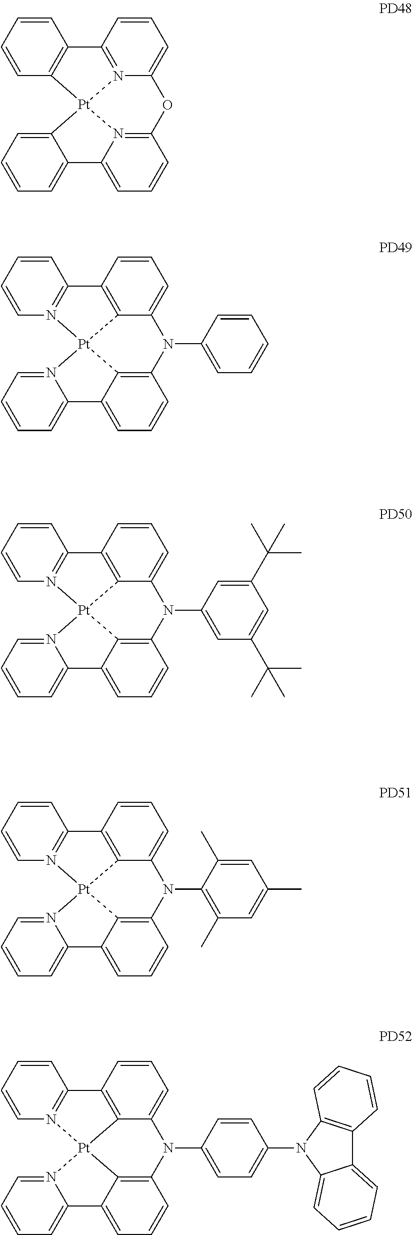

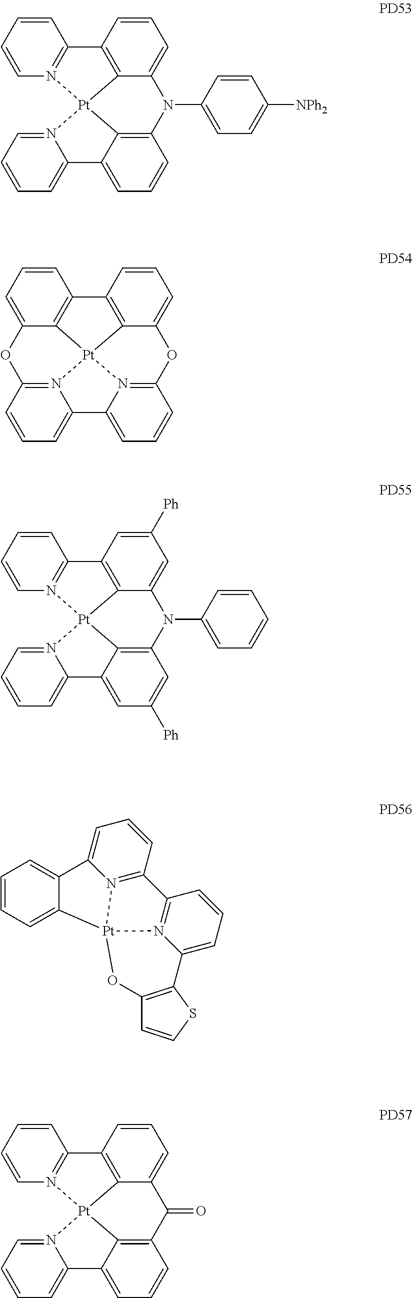

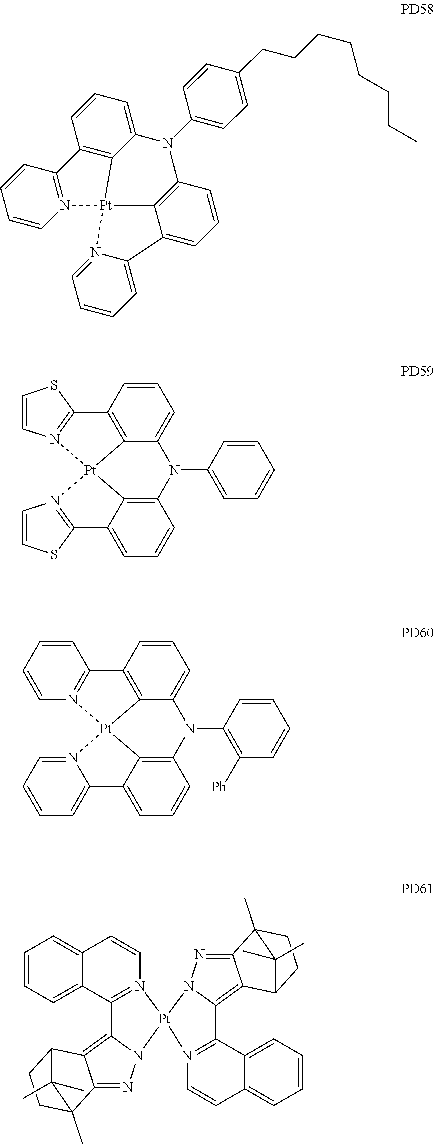

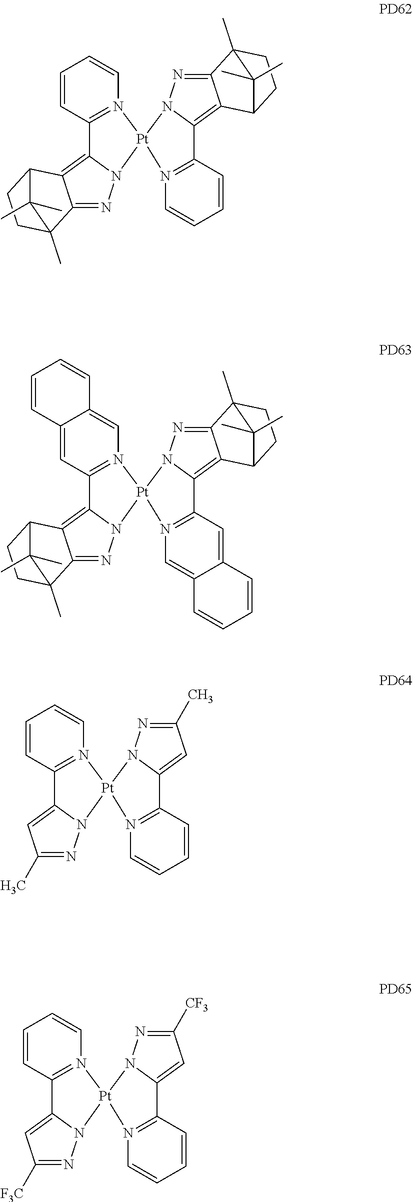

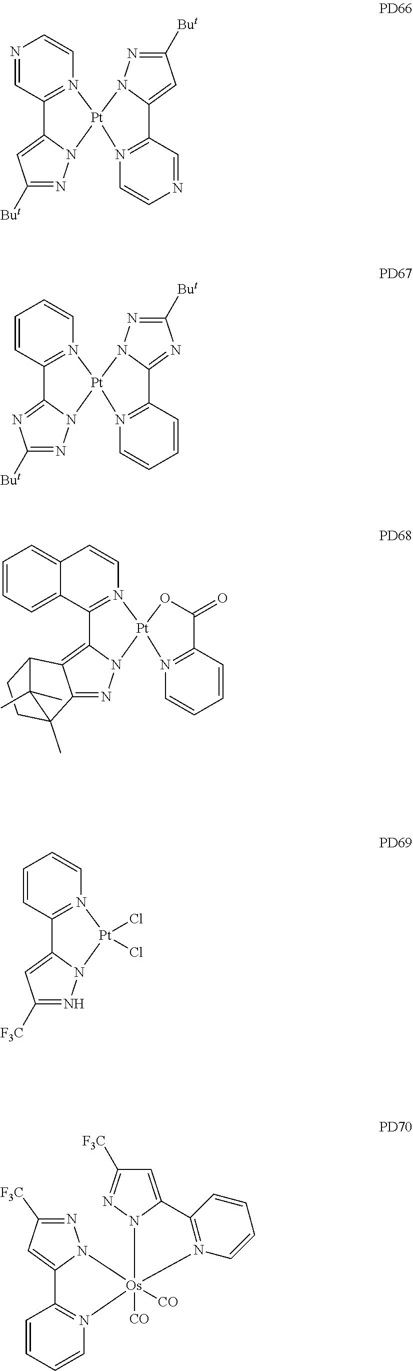

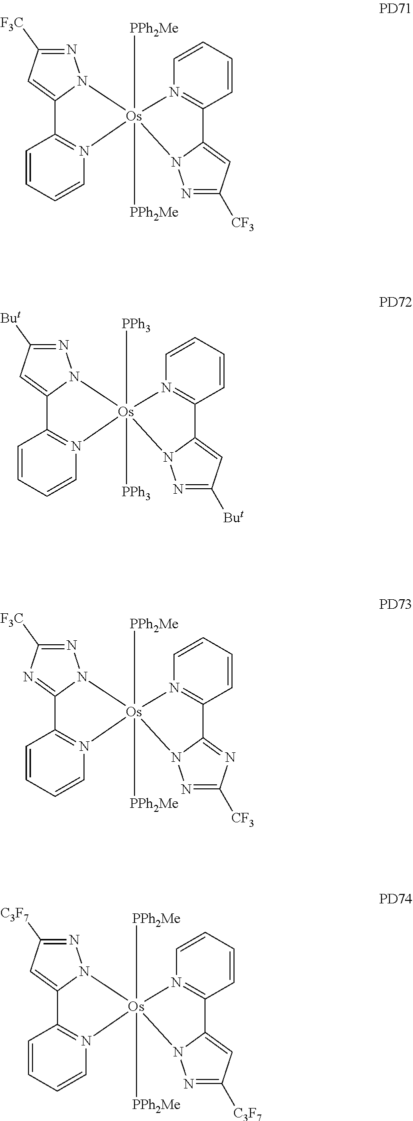

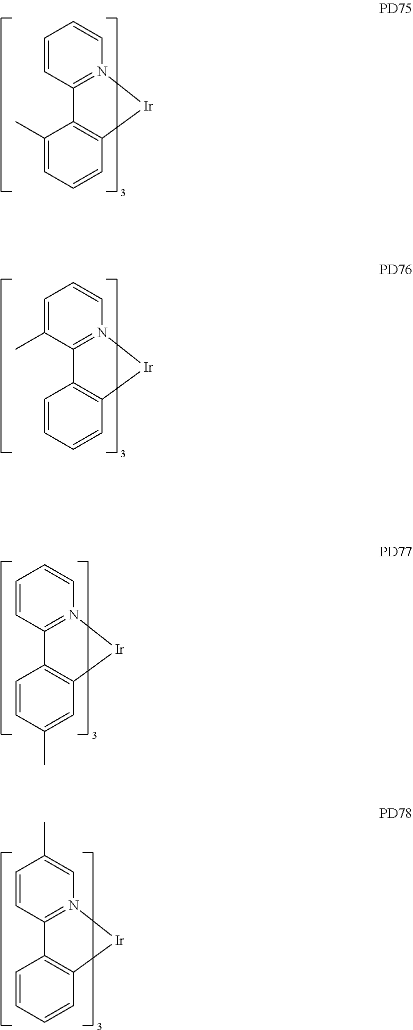

In some embodiments, the phosphorescent dopant may include PtOEP or PhGD represented below:

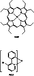

In some other embodiments, the phosphorescent dopant may include at least one of DPVBi, DPAVBi, TBPe, DCM, DCJTB, Coumarin 6, and C545T represented below.

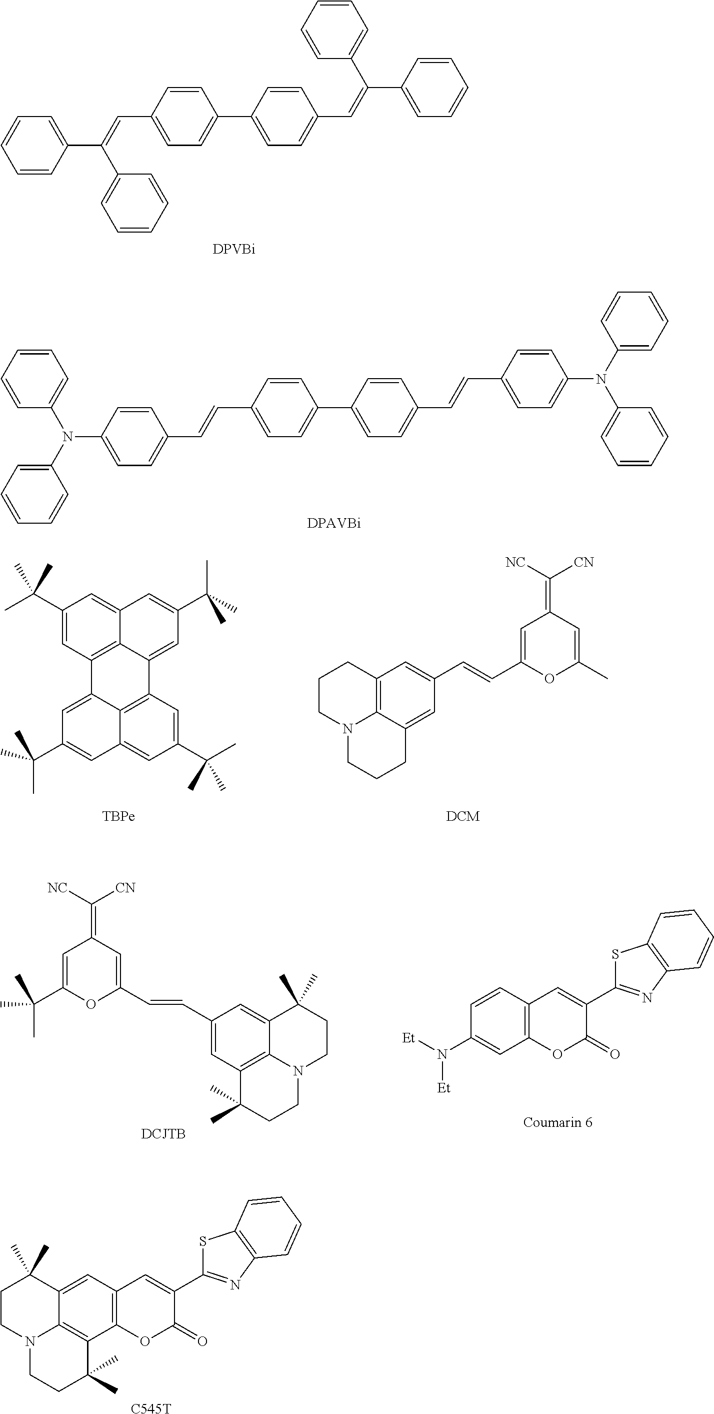

When the EML includes both a host and a dopant, the amount of the dopant may be from about 0.01 to about 20 parts by weight based on 100 parts by weight of the host. However, the amount of the dopant is not limited to this range.

The thickness of the EML may be about 100 Å to about 1000 Å, and in some embodiments, may be from about 200 Å to about 600 Å. When the thickness of the EML is within these ranges, the EML may have improved light emitting ability without a substantial increase in driving voltage.

Next, the electron transport region may be disposed on the EML.

The electron transport region may include at least one of a HBL, an ETL, and an EIL.

In some embodiments, the electron transport region may have a structure including an ETL, a HBL/ETL/EIL, or an ETL/EIL, wherein the layers forming the structure of the electron transport region may be sequentially stacked on the EML in the stated order. However, embodiments of the present disclosure are not limited thereto. For example, an organic light-emitting device according to one embodiment may include at least two hole transport layers in the hole transport region, and in this case, a hole transport layer contacting the emission layer is defined to be a hole transport auxiliary layer.

The ETL may have a single-layer structure or a multi-layer structure including at least two different materials.

The electron transport region may include a condensed cyclic compound represented by Formula 1 above. For example, the electron transport region may include an ETL, and the ETL may include the condensed cyclic compound of Formula 1 above. More specifically, the electron transport auxiliary layer may include the condensed cyclic compound represented by the Formula 1.

The organic light-emitting device may further include a hole transport auxiliary layer including a compound represented by he following Formula 2, with the electron transport layer including the condensed cyclic compound.

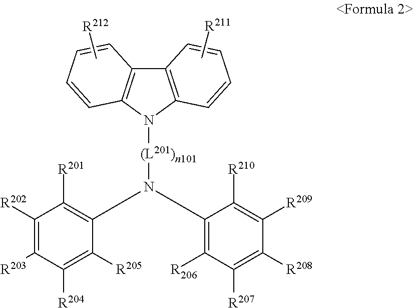

In Formula 2,

L²⁰¹ is a substituted or unsubstituted C6 to C30 arylene group, or a substituted or unsubstituted C2 to C30 heteroarylene group,

n101 is an integer of 1 to 5,

R²⁰¹ to R²¹² are each independently hydrogen, a deuterium, a substituted or unsubstituted C1 to C20 alkyl group, a substituted or unsubstituted C6 to C50 aryl group, a substituted or unsubstituted C2 to C50 heteroaryl group or a combination thereof, and

R²⁰¹ to R²¹² are each independently present or are fused to each other to form a ring.

In Formula 2, “substituted” refers to one substituted with deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C3 to C30 cycloalkyl group, a C2 to C30 heterocycloalkyl group, a C6 to C30 aryl group, a C2 to C30 heteroaryl group, a C1 to 020 alkoxy group, a fluoro group, a C1 to C10 trifluoroalkyl group or a cyano group, instead of at least one hydrogen.

A hole transport auxiliary layer according to one embodiment may include one of compounds represented by the following Formula P-1 to P-5.

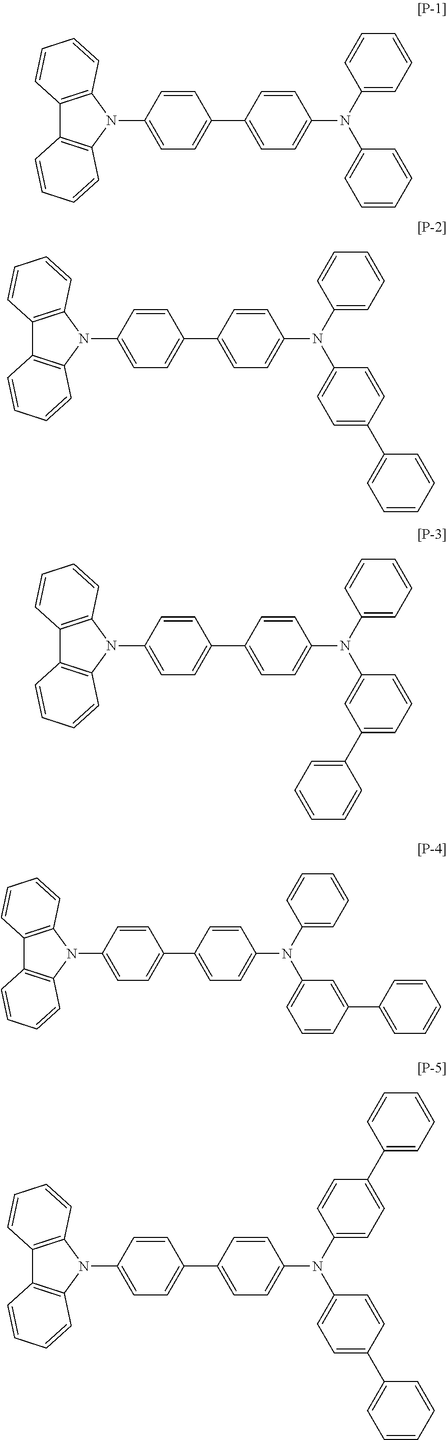

Conditions for forming the HBL, ETL, and EIL of the electron transport region may be defined based on the above-described formation conditions for the HIL.

When the electron transport region includes the HBL, the HBL may include at least one of BCP and Bphen below and Bphen below. However, embodiments of the present disclosure are not limited thereto.

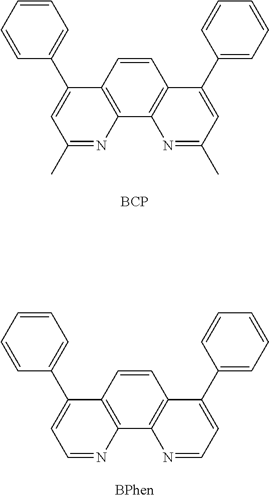

The thickness of the HBL may be from about 20 Å to about 1000 Å, and in some embodiments, from about 30 Å to about 300 Å. When the thickness of the HBL is within these ranges, the HBL may have improved hole blocking ability without a substantial increase in driving voltage.

The ETL may further include at least one of Alq₃, Balq, TAZ, and NTAZ below, in addition to BCP and Bphen described above.

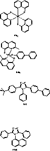

In some embodiments, the ETL may include at least one of Compounds ET1 and ET2 represented below, but is not limited thereto.

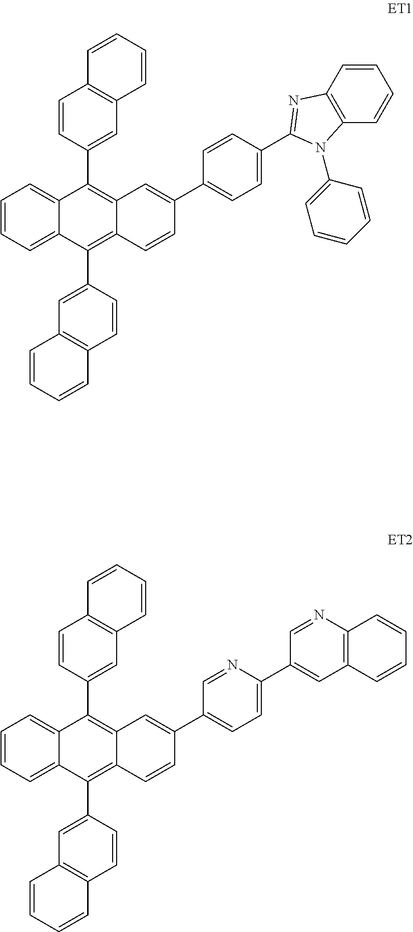 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1]

In some other embodiments, the ETL may include the condensed cyclic compound of Formula 1 above, but is not limited thereto.

A thickness of the ETL may be from about 100 Å to about 1000 Å, and in some embodiments, from about 150 Å to about 500 Å. When the thickness of the ETL is within these ranges, the ETL may have satisfactory electron transporting ability without a substantial increase in driving voltage.

In some embodiments the ETL may further include a metal-containing material, in addition to the above-described materials.

The metal-containing material may include a lithium (Li) complex. Non-limiting examples of the Li complex are compound ET-D1 below (lithium quinolate (LiQ)), or compound ET-D2 below.

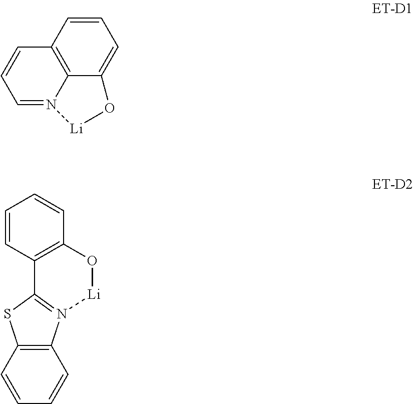

The electron transport region may include an EIL that may facilitate injection of electrons from the second electrode 19. The EIL may include at least one selected from LiF, NaCl, CsF, Li₂O, and BaO. The thickness of the EIL may be from about 1 Å to about 100 Å, and in some embodiments, from about 3 Å to about 90 Å. When the thickness of the EIL is within these ranges, the EIL may have satisfactory electron injection ability without a substantial increase in driving voltage.

The second electrode 19 is disposed on the organic layer 15. The second electrode 19 may be a cathode. A material for the second electrode 19 may be a metal, an alloy, or an electrically conductive compound that have a low work function, or a combination thereof. Non-limiting examples of the material for the second electrode 19 are lithium (Li), magnesium (Mg), aluminum (Al), aluminum (Al)-lithium (Li), calcium (Ca), magnesium (Mg)-indium (In), and magnesium (Mg)-silver (Ag), or the like. In some embodiments, to manufacture a top-emission light-emitting device, the second electrode 19 may be formed as a transmissive electrode from, for example, indium tin oxide (ITO) or indium zinc oxide (IZO).

Although the organic light-emitting device of FIG. 1 is described above, embodiments of the present disclosure are not limited thereto.

As used herein, a C₁-C₆₀ alkyl group refers to a linear or branched aliphatic hydrocarbon monovalent group having 1 to 60 carbon atoms. Non-limiting examples of the C₁-C₆₀ alkyl group a methyl group, an ethyl group, a propyl group, an isobutyl group, a sec-butyl group, a tert-butyl group, a pentyl group, an iso-amyl group, and a hexyl group. A C₁-C₆₀ alkenylene group refers to a divalent group having the same structure as the C₁-C₆₀ alkyl.

As used herein, a C₁-C₆₀ alkoxy group refers to a monovalent group represented by —OA₁₀₁ (where A₁₀₁ is a C₁-C₆₀ alkyl group as described above. Non-limiting examples of the C₁-C₆₀ alkoxy group are a methoxy group, an ethoxy group, and an isopropyloxy group.

As used herein, a C₂-C₆₀ alkenyl group refers to a structure including at least one carbon double bond in the middle or terminal of the C₂-C₆₀ alkyl group. Non-limiting examples of the C₂-C₆₀ alkenyl group are an ethenyl group, a prophenyl group, and a butenyl group. A C₂-C₆₀ alkenylene group refers to a divalent group having the same structure as the C₂-C₆₀ alkenyl group.

As used herein, a C₂-C₆₀ alkynyl group refers to a structure including at least one carbon triple bond in the middle or terminal of the C₂-C₆₀ alkyl group. Non-limiting examples of the C₂-C₆₀ alkynyl group are an ethynyl group and a propynyl group. A C₂-C₆₀ alkynylene group used herein refers to a divalent group having the same structure as the C₂-C₆₀ alkynyl group.

As used herein, a C₃-C₁₀ cycloalkyl group refers to a monovalent, monocyclic hydrocarbon group having 3 to 10 carbon atoms. Non-limiting examples of the C₃-C₁₀ cycloalkyl group are a cyclopropyl group, a cyclobutyl group, a cyclopentyl group, a cyclohexyl group, and a cycloheptyl group. A C₃-C₁₀ cycloalkenylene group refers to a divalent group having the same structure as the C₃-C₁₀ cycloalkyl group.

As used herein, a C₁-C₁₀ heterocycloalkyl group refers to a monovalent monocyclic group having 1 to 10 carbon atoms in which at least one hetero atom selected from N, O, P, and S is included as a ring-forming atom. Non-limiting examples of the C₁-C₁₀ heterocycloalkyl group are a tetrahydrofuranyl group and a tetrahydrothiophenyl group. A C₁-C₁₀ heterocycloalkenylene group refers to a divalent group having the same structure as the C₁-C₁₀ heterocycloalkyl group.

As used herein, a C₃-C₁₀ cycloalkenyl group refers to a monovalent monocyclic group having 3 to 10 carbon atoms that includes at least one double bond in the ring but does not have aromaticity. Non-limiting examples of the C₃-C₁₀ cycloalkenyl group are a cyclopentenyl group, a cyclohexenyl group, and a cycloheptenyl group. A C₃-C₁₀ cycloalkenylene group refers to a divalent group having the same structure as the C₃-C₁₀ cycloalkenyl group.

As used herein, a C₁-C₁₀ heterocycloalkenyl group used herein refers to a monovalent monocyclic group having 1 to 10 carbon atoms that includes at least one double bond in the ring and in which at least one hetero atom selected from N, O, P, and S is included as a ring-forming atom. Non-limiting examples of the C₁-C₁₀ heterocycloalkenyl group are a 2,3-hydrofuranyl group and a 2,3-hydrothiophenyl group. A C₁-C₁₀ heterocycloalkenylene group used herein refers to a divalent group having the same structure as the C₁-C₁₀ heterocycloalkenyl group.

As used herein, a C₆-C₆₀ aryl group refers to a monovalent, aromatic carbocyclic aromatic group having 6 to 60 carbon atoms, and a C₆-C₆₀ arylene group refers to a divalent, aromatic carbocyclic group having 6 to 60 carbon atoms. Non-limiting examples of the C₆-C₆₀ aryl group are a phenyl group, a naphthyl group, an anthracenyl group, a phenanthrenyl group, a pyrenyl group, and a chrysenyl group. When the C₆-C₆₀ aryl group and the C₆-C₆₀ arylene group include at least two rings, the rings may be fused to each other.

As used herein, a C₂-C₆₀ heteroaryl group refers to a monovalent, aromatic carbocyclic aromatic group having 2 to 60 carbon atoms in which at least one hetero atom selected from N, O, P, and S is included as a ring-forming atom, and 2 to 60 carbon atoms. A C₂-C₆₀ heteroarylene group refers to a divalent, aromatic carbocyclic group having 2 to 60 carbon atoms in which at least one hetero atom selected from N, O, P, and S is included as a ring-forming atom. Non-limiting examples of the C₂-C₆₀ heteroaryl group are a pyridinyl group, a pyrimidinyl group, a pyrazinyl group, a pyridazinyl group, a triazinyl group, a quinolinyl group, and an isoquinolinyl group. When the C₂-C₆₀ heteroaryl and the C₂-C₆₀ heteroarylene include at least two rings, the rings may be fused to each other.

As used herein, a C₆-C₆₀ aryloxy group indicates —OA₁₀₂ (where A₁₀₂ is a C₆-C₆₀ aryl group as described above), and a C₆-C₆₀ arylthio group indicates —SA₁₀₃ (where A₁₀₃ is a C₆-C₆₀ aryl group as described above).

As used herein, a monovalent non-aromatic condensed polycyclic group refers to a monovalent group having at least two rings condensed to each other, in which only carbon atoms (for example, 8 to 60 carbon atoms) are exclusively included as ring-forming atoms and the entire molecule has non-aromaticity. A non-limiting example of the monovalent non-aromatic condensed polycyclic group is a fluorenyl group. A divalent non-aromatic condensed polycyclic group refers to a divalent group having the same structure as the monovalent non-aromatic condensed polycyclic group.

As used herein, a monovalent non-aromatic condensed heteropolycyclic group refers to a monovalent group having at least two rings condensed to each other, in which carbon atoms (for example, 1 to 60 carbon atoms) and a hetero atom selected from N, O, P, and S are as ring-forming atoms and the entire molecule has non-aromaticity. A non-limiting example of the monovalent non-aromatic condensed heteropolycyclic group is a carbazolyl group. A divalent non-aromatic condensed heteropolycyclic group refers to a divalent group having the same structure as the monovalent non-aromatic condensed heteropolycyclic group.

The “biphenyl” means “phenyl group substituted with phenyl group”.

One or more embodiments of the present disclosure, which include condensed cyclic compounds, and organic light-emitting devices including the same, will now be described in detail with reference to the following examples. However, these examples are only for illustrative purposes and are not intended to limit the scope of the one or more embodiments of the present disclosure. In the following synthesis example, the expression that “‘B’ instead of ‘A’ was used” means that the amounts of ‘B’ and ‘A’ were the same in equivalent amounts.

Hereinafter, a starting material and a reaction material used in Examples and Synthesis Examples were purchased from Sigma-Aldrich Co. Ltd. or TCI Inc. unless there was particularly mentioned.

### EXAMPLES

### Synthesis of Boronic Ester

Boronic ester of the following Synthesis Example was synthesized according to the same method as a synthesis method described on page 35 of KR 10-2014-0135524A, and the reaction scheme of the boronic ester are provided as [General Formula A] and [General Formula B].

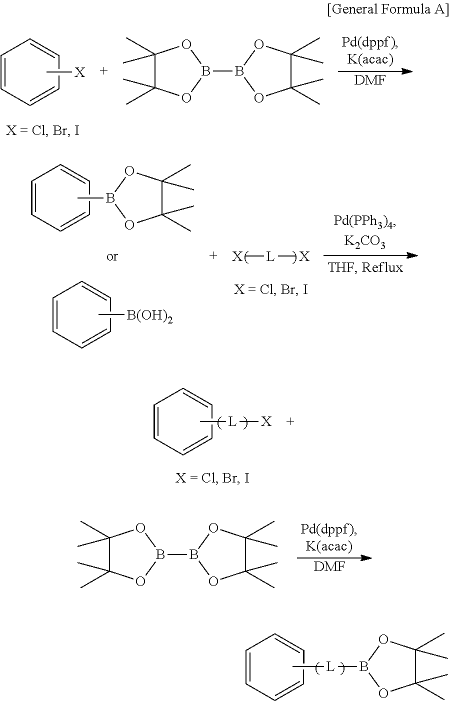

In General Formula A, “L” is a substituted or unsubstituted C6 to C60 arylene group.

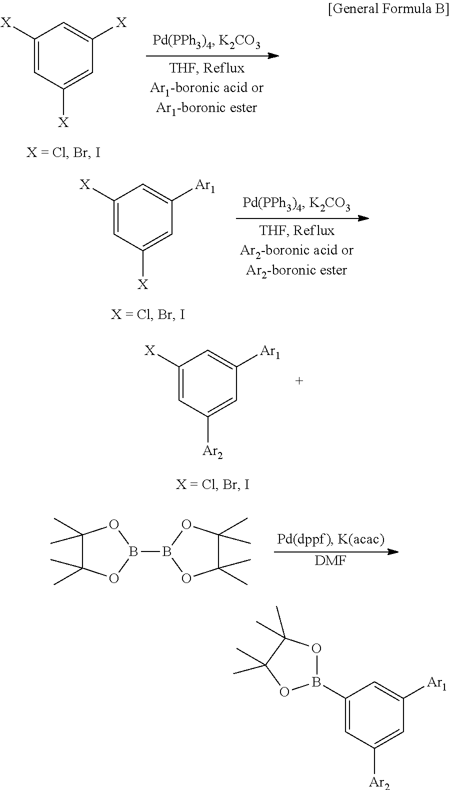

In General Formula B, Ar₁ and Ar₂ are independently a substituted or unsubstituted C6 to C30 aryl group. For example, Ar₁ and Ar₂ may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted fluoranthenyl group, a substituted or unsubstituted chrysenyl group, and the like.)

Hereinafter, a method of synthesizing the boronic ester as a reaction material used in the present invention was illustrated by taking an example for better understanding.

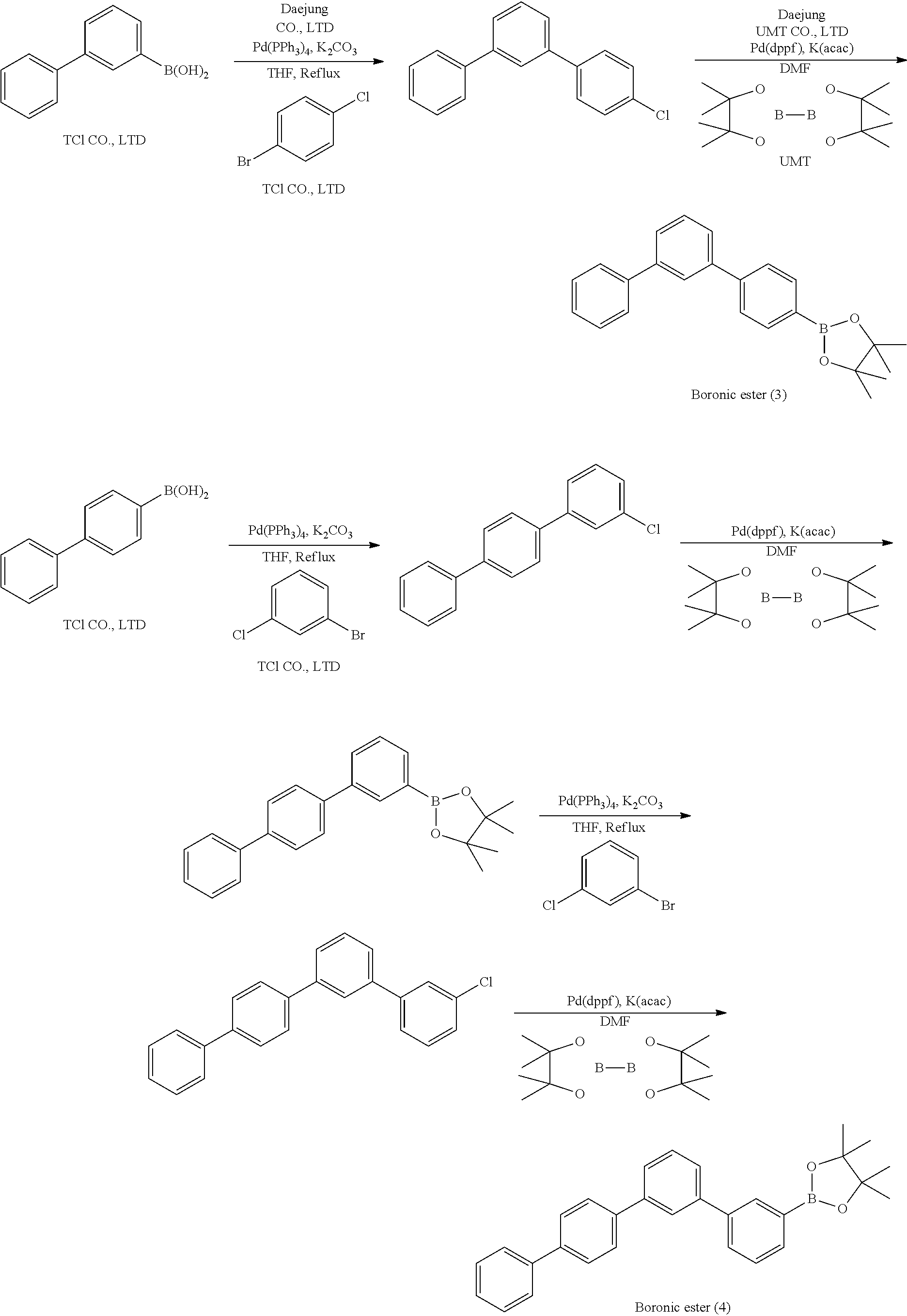

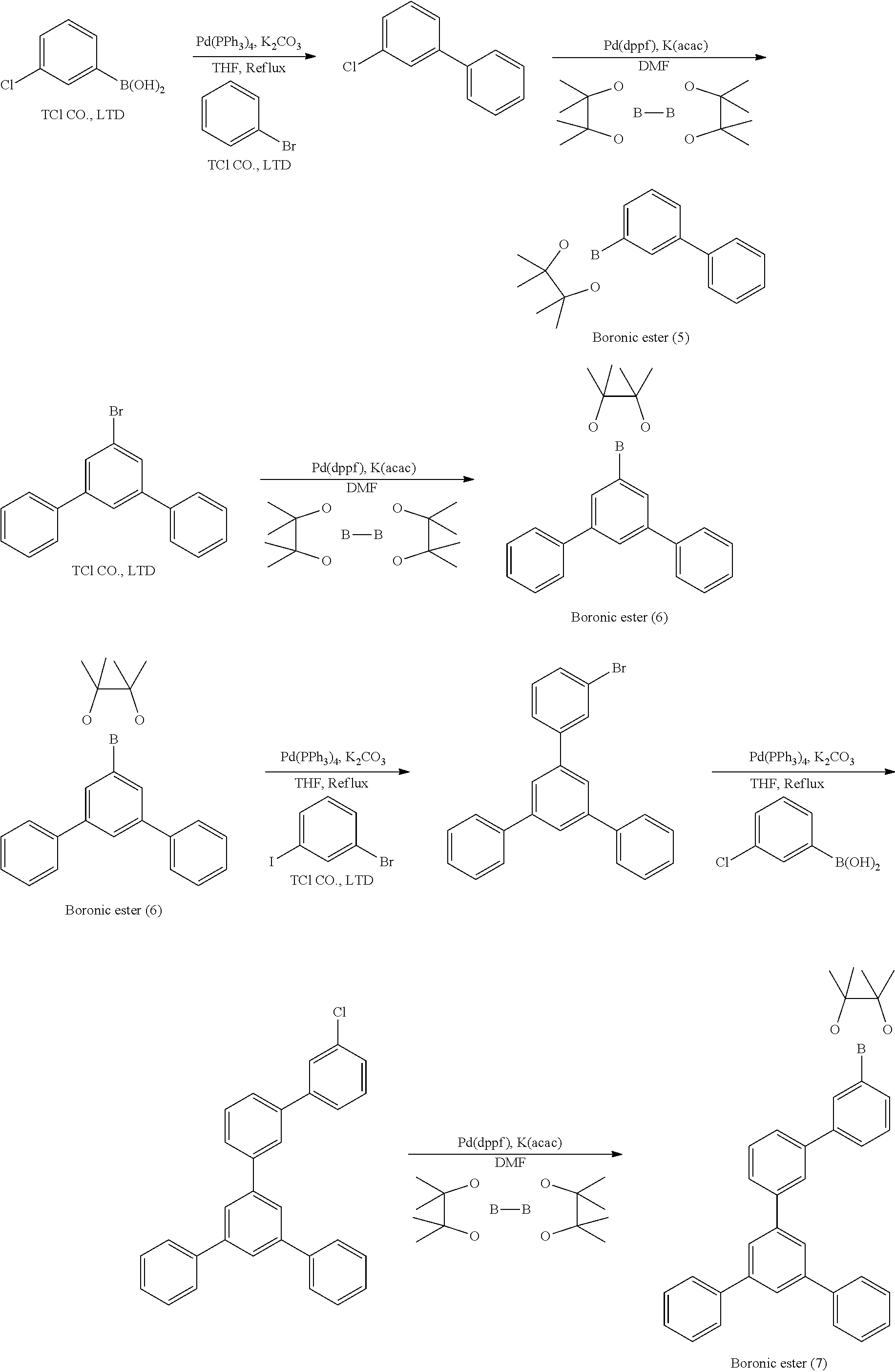

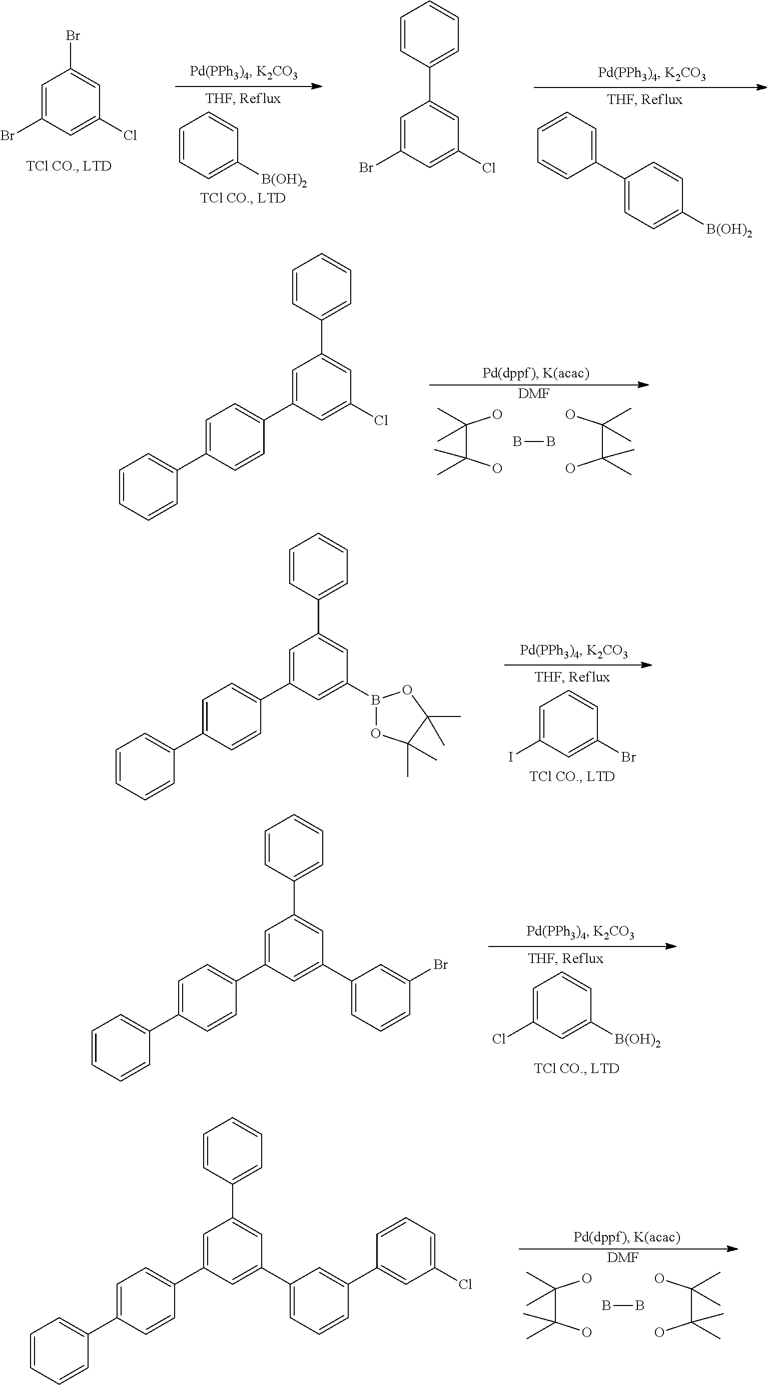

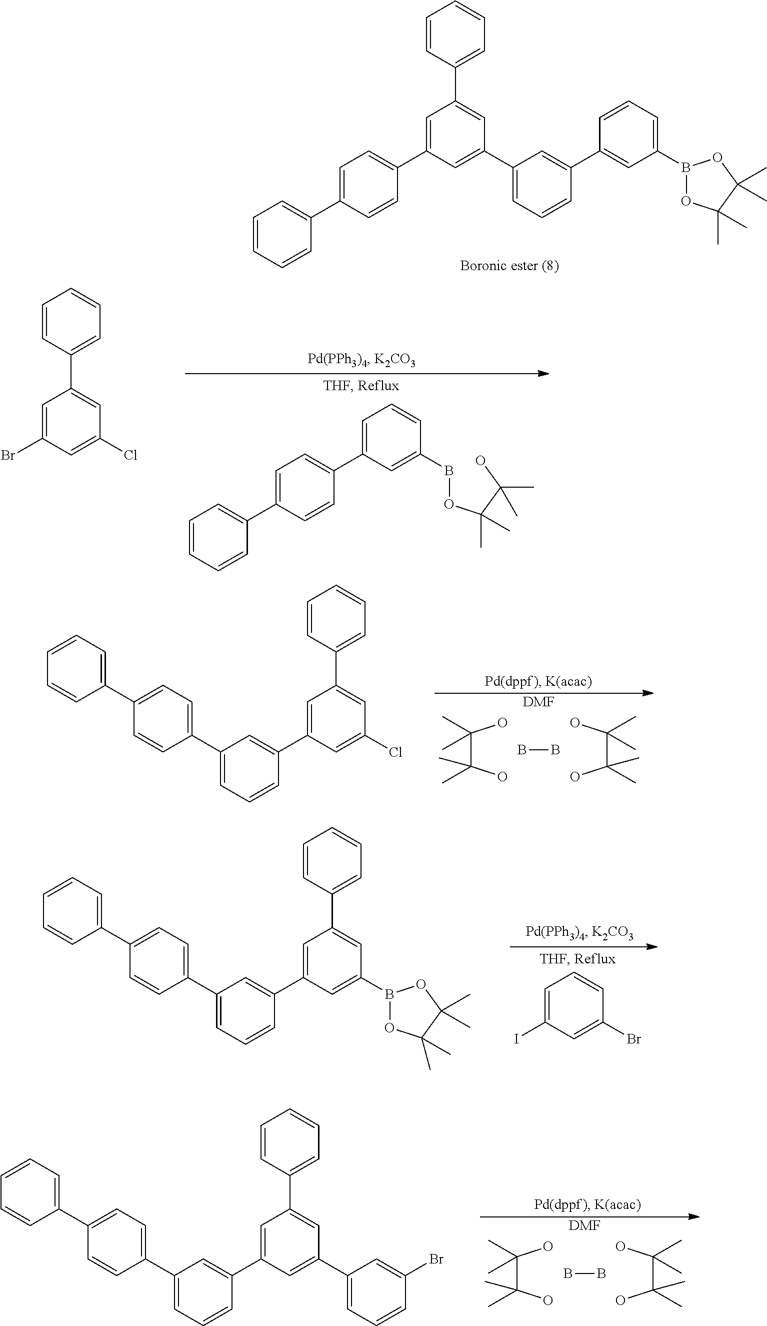

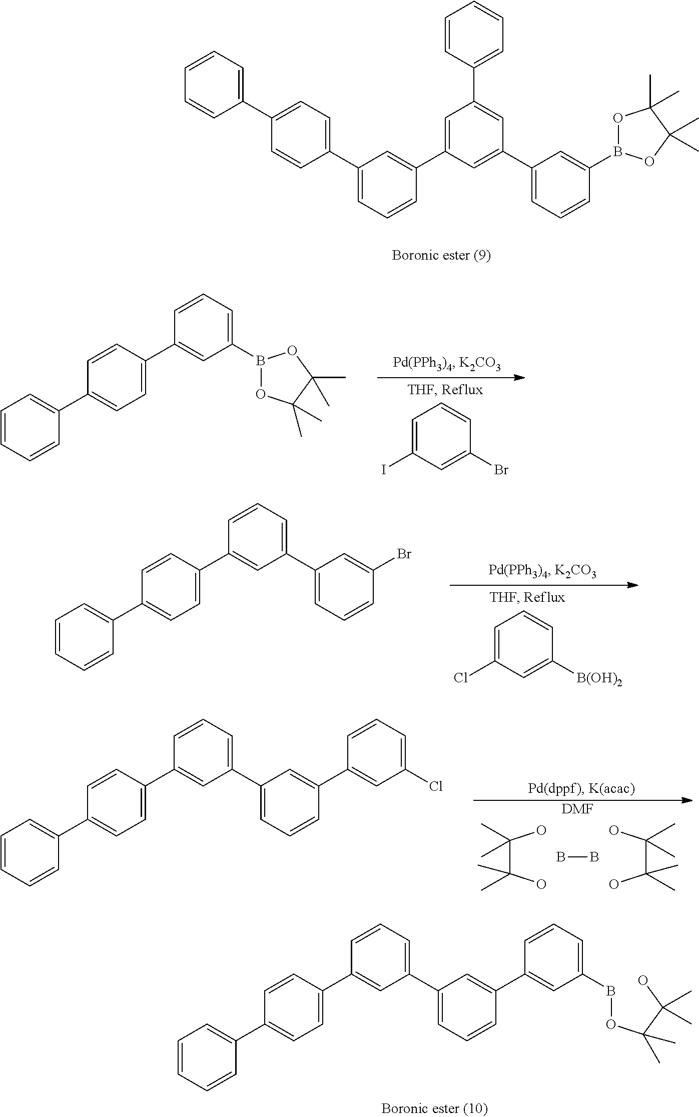

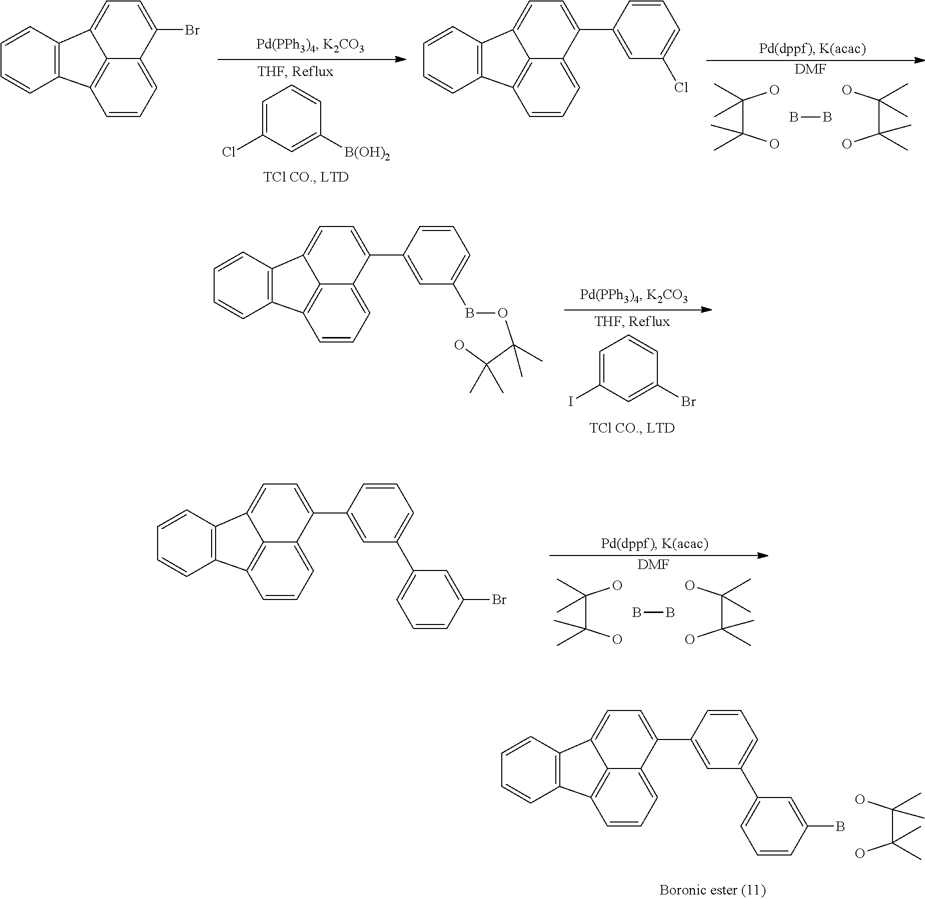

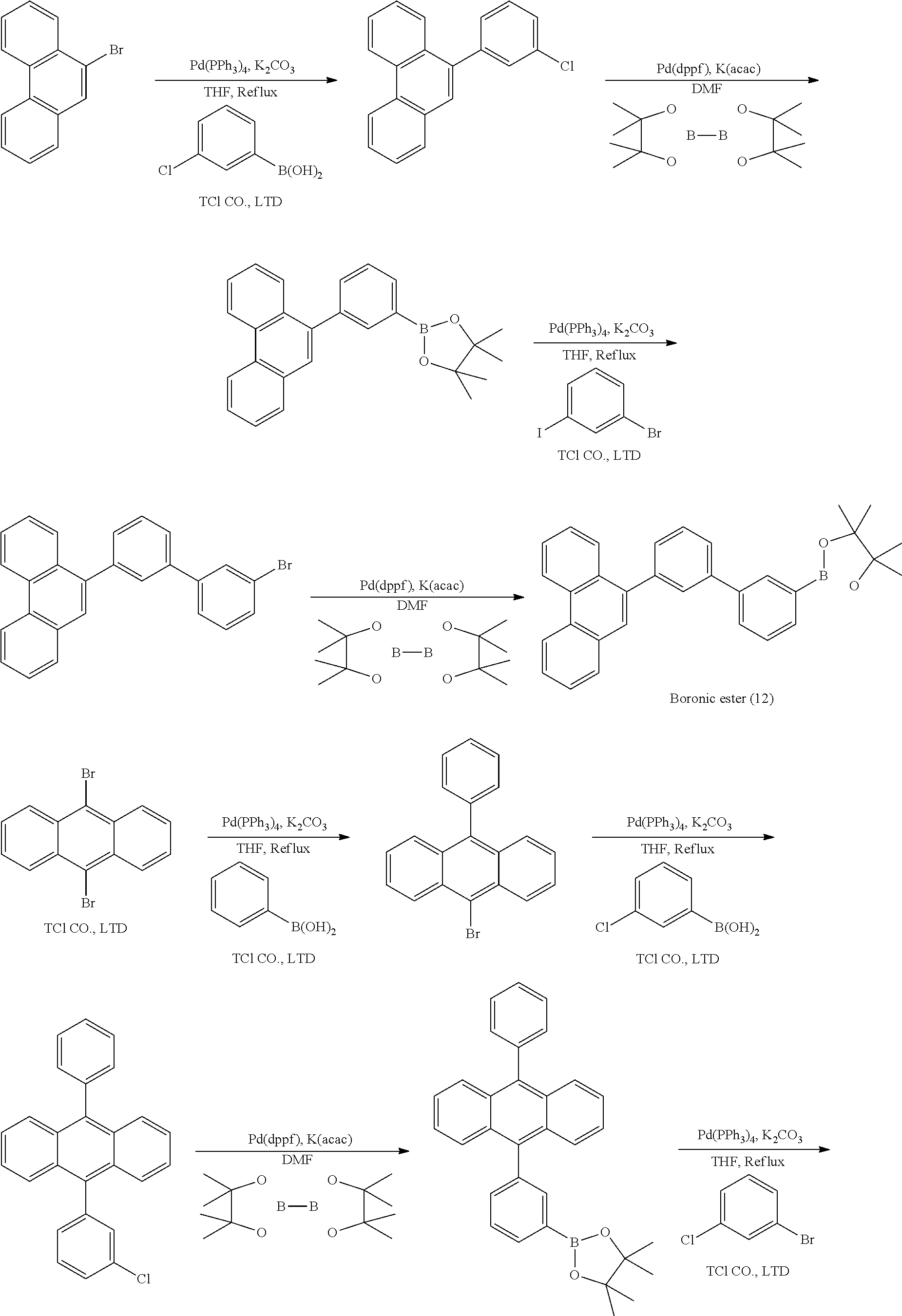

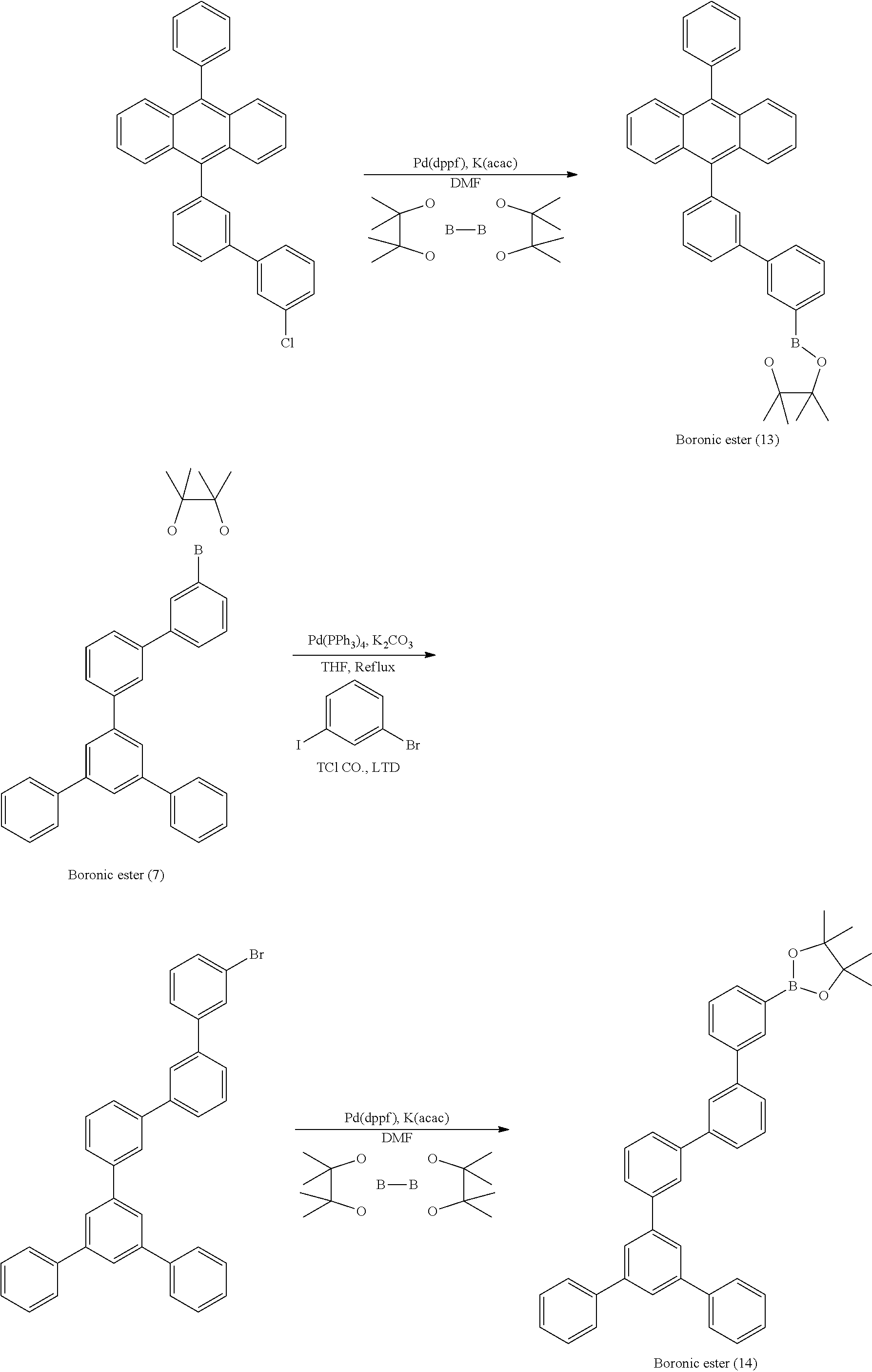

### Synthesis of First Host Compound

### Synthesis Example 1

### Synthesis of Compound 29

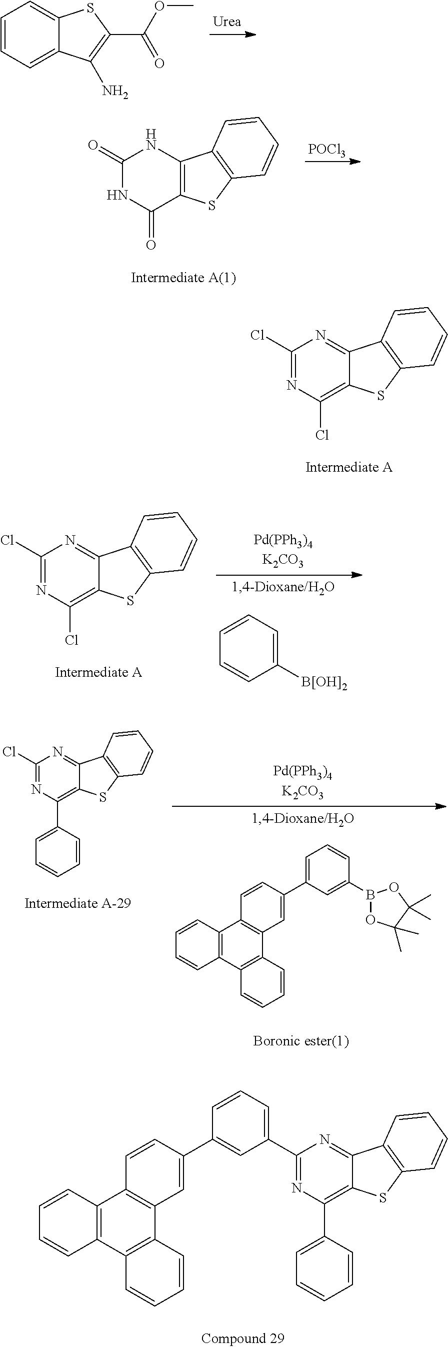

### Synthesis of Intermediate A(1)(benzo-1H-thieno[3,2-d]pyrimidine-2,4-dione)

A mixture of 47.5 g (0.23 mol) of benzo-methyl 3-amino-2-thiophenecarboxylate and 79.4 g (1.15 mol) of urea was stirred in a 2000-mL round-bottom flask at about 200° C. for about 2 hours. After the high-temperature reaction product was cooled down to room temperature, a sodium hydroxide solution was added thereto, followed by filtration to remove impurities and acidification with HCl. The resulting precipitate was dried to obtain Intermediate A(1) (35 g, Yield: 75%).

calcd. C₁₀H₆N₂C₂S: C, 55.04; H, 2.77; N, 12.84; O, 14.66; S, 14.69. found: C, 55.01; H, 2.79; N, 12.81; O, 14.69; S, 14.70.

### Synthesis of Intermediate A (benzo-2,4-dichloro-thieno[3,2-d]pyrimidine)

35 g (0.16 mol) of Intermediate A(1) (benzo-1H-thieno [3,2-d]pyrimidine-2,4-dione) and 600 mL of phosphorus oxychloride were mixed in a 1000-mL round-bottom flask and stirred under reflux for about 6 hours. The reaction product was cooled down to room temperature, and poured into ice/water with stirring to obtain a precipitate. The resulting reaction precipitate was filtered to obtain Intermediate A ((benzo-2,4-dichloro-thieno [3,2-d]pyrimidine) in white solid form (35 g, Yield: 85%). Intermediate A was identified using elemental analysis and nuclear magnetic resonance (NMR). The results are as follows.

calcd. C₁₀H₄Cl₂N₂S: C, 47.08; H, 1.58; Cl, 27.79; N, 10.98; S, 12.57. found: C, 47.03; H, 1.61; Cl, 27.81; N, 10.98; S, 12.60.

300 MHz (CDCl₃, ppm): 7.63 (t, 1H), 7.76 (t, 4H), 7.95 (d, 1H), 8.53 (d, 1H)

### Synthesis of Intermediate A-29

20.0 g (78.4 mmol) of Intermediate A, 11.0 g (90.15 mmol) of phenylboronic acid, 27.09 g (195.99 mmol) of potassium carbonate, and 4.53 g (3.9 mmol) of tetrakis-(triphenylphosphine)palladium(0) (Pd(PPh₃)₄) were added to 300 mL of 1,4-dioxane and 150 mL of water in a 1000-mL flask, and heated in a nitrogen atmosphere at about 60□ for about 12 hours. The resulting mixture was added to 1000 mL of methanol to obtain crystalline solid powder by filtering. The resulting product was dissolved in monochlorobenzene and filtered using Silica gel/Celite, followed by removing an appropriate amount of the organic solvent and recrystallization with methanol to obtain Intermediate A-29 (13.9 g, Yield: 60%).

calcd. C₁₆H₉ClN₂S: C, 64.75; H, 3.06; Cl, 11.95; N, 9.44; S, 10.80. found: C, 63.17; H, 3.08; Cl, 12.13; N, 9.37; S, 10.82.

### Synthesis of Compound 29

13.9 g (46.8 mmol) of Intermediate A-29, 23.2 g (53.86 mmol) of triphenylene-phenyl-boronic ester, 16.2 g (117.1 mmol) of potassium carbonate, and 2.7 g (2.3 mmol) of tetrakis-(triphenylphosphine)palladium(0) (Pd(PPh₃)₄) were added to 150 mL of 1,4-dioxane 150 mL and 75 mL of water in a 500-mL round-bottom flask, and heated under reflux in a nitrogen atmosphere for about 6 hours. The resulting mixture was added to 500 mL of methanol to obtain crystalline solid powder by filtering. The resulting product was dissolved in monochlorobenzene and filtered using Silica gel/Celite, followed by removing an appropriate amount of the organic solvent and recrystallization with methanol to obtain Compound 29 (16.7 g, Yield: 64%). Compound 29 was identified using elemental analysis and nuclear magnetic resonance (NMR). The results are as follows.

calcd. C₄₀H₂₄N₂S: C, 85.08; H, 4.28; N, 4.96; S, 5.68. found: C, 84.95; H, 4.18; N, 5.17; S, 5.72.

300 MHz (CDCl₃, ppm): 7.61-7.73 (m, 10H), 8.07 (t, 2H), 8.16 (d, 1H), 8.28 (d, 1H), 8.65 (t, 1H), 8.74 (s, 3H), 8.85-8.92 (m, 2H), 9.04 (s, 2H)

### Synthesis Example 2

### Synthesis of Compound 30

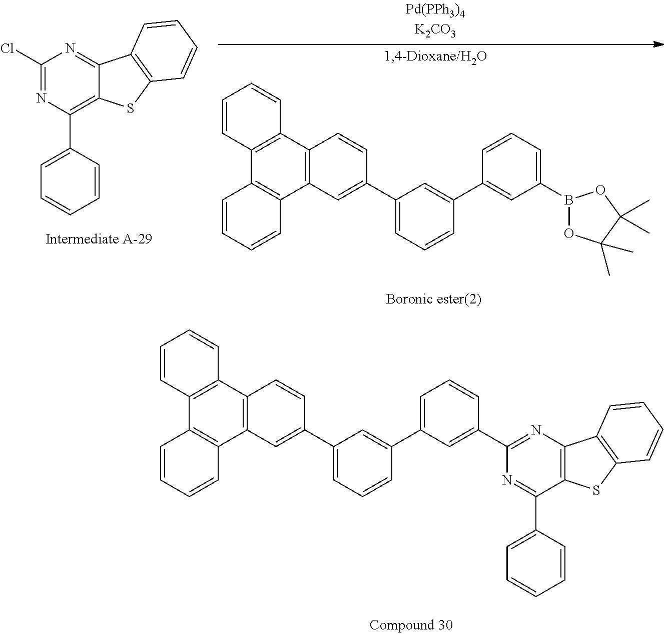

10.0 g (33.7 mmol) of Intermediate A-29, 19.6 g (38.8 mmol) of triphenylene-biphenyl-boronic ester (boronic ester(2), synthesis is described in the publication of KR 10-2014-0135524, page 37), 11.6 g (84.2 mmol) of potassium carbonate, 1.9 g (1.68 mmol) of tetrakis-(triphenylphosphine)palladium(0) (Pd(PPh₃)₄) were added to 100 mL of 1,4-dioxane and 50 mL of water in a 250-mL round-bottom flask, and heated under reflux in a nitrogen atmosphere for about 6 hours. The resulting mixture was added to 300 mL of methanol to obtain crystalline solid powder by filtering. The resulting product was dissolved in monochlorobenzene and filtered using Silica gel/Celite, followed by removing an appropriate amount of the organic solvent and recrystallization with methanol to obtain Compound 30 (14.0 g, Yield: 65%). Compound 30 was identified using elemental analysis and nuclear magnetic resonance (NMR). The results are as follows.

calcd. C₄₆H₂₈N₂S: C, 86.22; H, 4.40; N, 4.37; S, 5.00. found: C, 85.95; H, 4.58; N, 4.17; S, 5.02.

300 MHz (CDCl₃, ppm): 7.63-7.91 (m, 12H), 8.05 (d, 1H), 8.10 (d, 1H), 8.18 (d, 1H), 8.27 (d, 1H), 8.33 (s, 1H), 8.39 (dd, 2H), 8.77 (t, 2H), 8.81-8.92 (m, 3H), 8.95 (d, 1H), 9.08-9.12 (m, 2H), 9.20 (s, 1H)

### Synthesis Example 3

### Synthesis of Compound 27

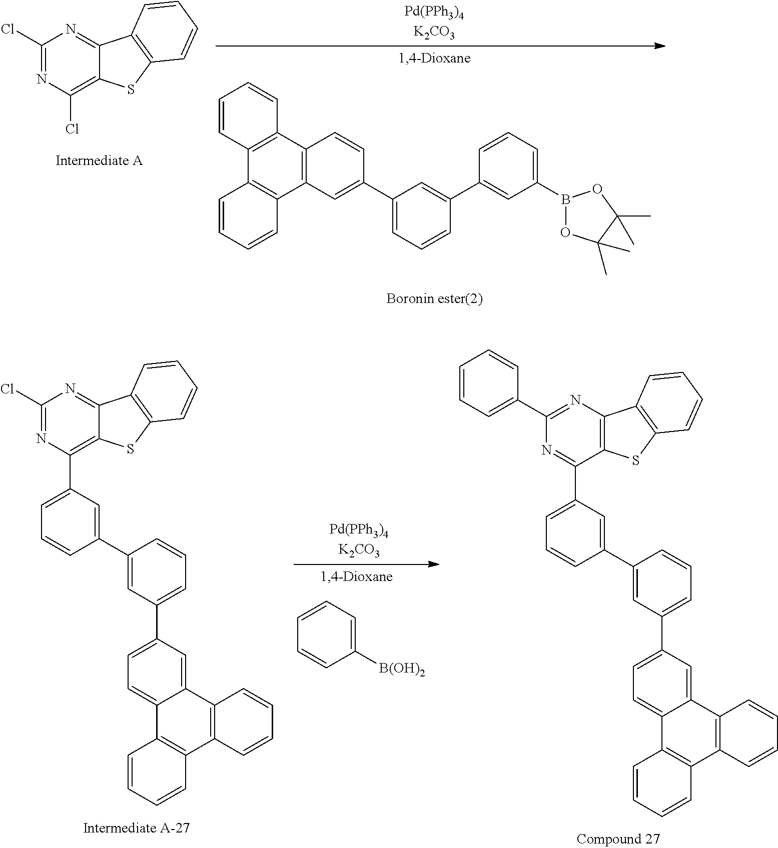 [SMILES: Clc1nc(Cl)c2sc3ccccc3c2n1 ; CC1(C)OB(c2cccc(-c3cccc(-c4ccc5c6ccccc6c6ccccc6c5c4)c3)c2)OC1(C)C ; Clc1nc(-c2cccc(-c3cccc(-c4ccc5c6ccccc6c6ccccc6c5c4)c3)c2)c2sc3ccccc3c2n1 ; OB(O)c1ccccc1 ; c1ccc(-c2nc(-c3cccc(-c4cccc(-c5ccc6c7ccccc7c7ccccc7c6c5)c4)c3)c3sc4ccccc4c3n2)cc1]

### Synthesis of Intermediate A-27

Intermediate A-27 (25.34 g, Yield: 68%) was synthesized in the same manner as in the synthesis of Intermediate A-29 in Synthesis Example 1, except that boronic ester(2) (triphenylene-biphenyl-boronic ester) instead of phenylboronic acid was used.

calcd. C₄₀H₂₃ClN₂S: C, 80.19; H, 3.87; Cl, 5.92; N, 4.68; S, 5.35. found: C, 78.57; H, 3.39; Cl, 5.68; N, 4.32; S, 5.15.

### Synthesis of Compound 27

Compound 27 (15.37 g, Yield: 56%) was synthesized in the same manner as in the synthesis of Compound 29 in Synthesis Example 1, except that Intermediate A-27 and phenylboronic acid, instead of Intermediate A-29 and triphenylene-biphenyl-boronic ester, respectively, were used.

calcd. C₄₆H₂₈N₂S: C, 86.22; H, 4.40; N, 4.37; S, 5.00. found: C, 85.18; H, 4.28; N, 4.14; S, 4.83.

300 MHz (CDCl₃, ppm): 7.41-7.57 (m, 10H), 7.70-7.88 (m, 7H), 7.98-8.18 (m, 6H), 8.28 (d, 2H), 8.93 (d, 2H), 9.15 (s, 1H)

### Synthesis Example ad-1

### Synthesis of Compound a-30

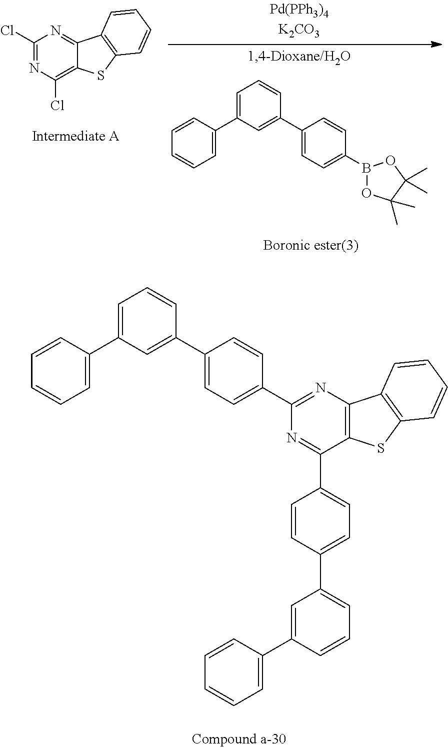 [SMILES: Clc1nc(Cl)c2sc3ccccc3c2n1 ; CC1(C)OB(c2ccc(-c3cccc(-c4ccccc4)c3)cc2)OC1(C)C ; c1ccc(-c2cccc(-c3ccc(-c4nc(-c5ccc(-c6cccc(-c7ccccc7)c6)cc5)c5sc6ccccc6c5n4)cc3)c2)cc1]

### Synthesis of Compound a-30

3.0 g (11.8 mmol) of the intermediate A, 8.8 g (24.7 mmol) of boronic acid (3), 4.1 g (29.4 mmol) of potassium carbonate, and 0.6 g (0.6 mmol) of tetrakis(triphenylphosphine)palladium (0) were put in a 100 mL round flask and then, heated under reflux in a nitrogen atmosphere for 6 hours. Then, a solid crystallized by adding the mixture obtained therefrom to 150 mL of methanol was filtered, dissolved in monochlorobenzene and filtered with silica gel/Celite, and then, recrystallized with methanol after removing an appropriate amount of a solvent therefrom, obtaining Compound a-30 (5.7 g, Yield: 75%). Elemental analysis result of Compound a-30 is as follows. The elemental analysis result of Compound a-30 is as follows.

calcd. C46H30N2S: C, 85.95; H, 4.70; N, 4.36; S, 4.99. found: C, 85.91; H, 4.69; N, 4.31; S, 4.94.

### Synthesis Example ad-2

### Synthesis of Compound a-40

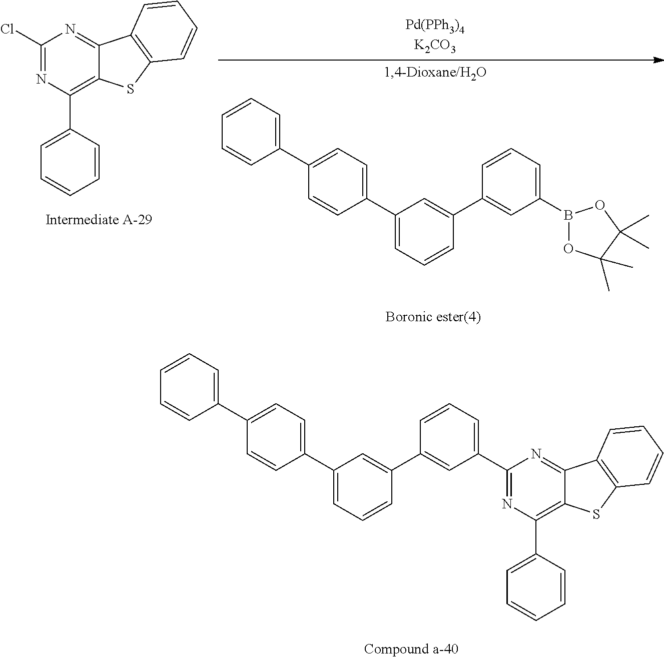 [SMILES: Clc1nc(-c2ccccc2)c2sc3ccccc3c2n1 ; CC1(C)OB(c2cccc(-c3cccc(-c4ccc(-c5ccccc5)cc4)c3)c2)OC1(C)C ; c1ccc(-c2ccc(-c3cccc(-c4cccc(-c5nc(-c6ccccc6)c6sc7ccccc7c6n5)c4)c3)cc2)cc1]

### Synthesis of Compound a-40

Compound a-40 was synthesized according to the same method as the Synthesis Example 1 of Compound 29 except for respectively using the intermediate A-29 and boronic ester (4). The elemental analysis result of the Compound a-40 was provided as follows.

calcd. C40H26N2S: C, 84.77; H, 4.62; N, 4.94; S, 5.66. found: C, 84.71; H, 4.59; N, 4.92; S, 5.60.

### Synthesis Example ad-3

### Synthesis of Compound a-41

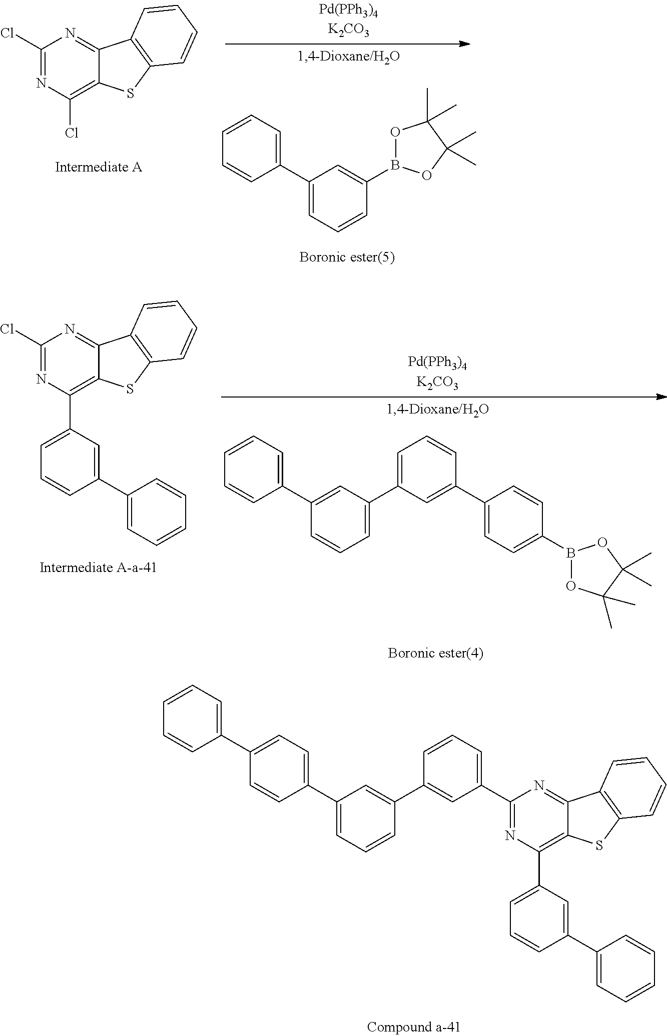

### Synthesis of Intermediate A-a-41

10.0 g (39.2 mmol) of the intermediate A, 12.1 g (43.1 mmol) of boronic acid (5), 13.5 g (98.0 mmol) of potassium carbonate, and 2.3 g (43.1 mmol) of tetrakis(triphenylphosphine)palladium (0) were added to 140 mL of dioxane and 70 mL of water in a 500 mL round flask and then heated under reflux in a nitrogen atmosphere at 60° C. for 12 hours. Then, a solid crystallized by adding the mixture obtained therefrom to 500 mL of methanol was filtered, dissolved in monochlorobenzene and filtered with silica gel/Celite, and then, recrystallized with methanol after removing an appropriate amount of a solvent therefrom, obtaining Intermediate A-a-41 (10.1 g, Yield: 69%).

calcd. C22H13ClN2S: C, 70.87; H, 3.51; Cl, 9.51; N, 7.51; S, 8.60. found: C, 70.80; H, 3.50; Cl, 9.47; N, 7.49; S, 8.60.

### Synthesis of Compound a-41

5.0 g (13.4 mmol) of the intermediate A-a-41, 6.4 g (14.8 mmol) of boronic ester (4), 4.6 g (33.5 mmol) of potassium carbonate, and 0.8 g (0.7 mmol) of tetrakis(triphenylphosphine)palladium (0) were added to 50 mL of dioxane and 25 mL of water in a 500 mL round flask and then heated under reflux in a nitrogen atmosphere for 8 hours. Then, a solid crystallized by adding the mixture obtained therefrom to 150 mL of methanol was filtered, dissolved in monochlorobenzene and filtered with silica gel/Celite, and then, recrystallized with methanol after removing an appropriate amount of a solvent therefrom, obtaining Compound a-30 (5.7 g, Yield: 75%). The elemental analysis result of Compound a-30 is as follows.

calcd. C46H30N2S: C, 85.95; H, 4.70; N, 4.36; S, 4.99. found: C, 85.90; H, 4.68; N, 4.31; S, 4.93.

### Synthesis Example ad-4

### Synthesis of Compound a-42

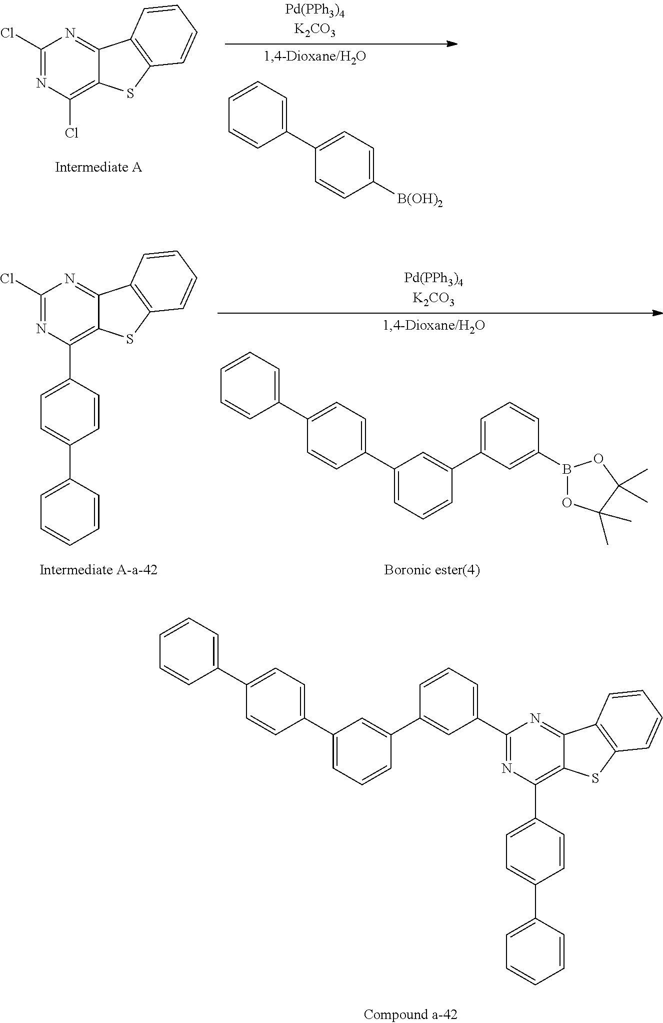 [SMILES: Clc1nc(Cl)c2sc3ccccc3c2n1 ; OB(O)c1ccc(-c2ccccc2)cc1 ; Clc1nc(-c2ccc(-c3ccccc3)cc2)c2sc3ccccc3c2n1 ; CC1(C)OB(c2cccc(-c3cccc(-c4ccc(-c5ccccc5)cc4)c3)c2)OC1(C)C ; c1ccc(-c2ccc(-c3cccc(-c4cccc(-c5nc(-c6ccc(-c7ccccc7)cc6)c6sc7ccccc7c6n5)c4)c3)cc2)cc1]

### Synthesis of Intermediate A-a-42

Intermediate A-a-42 (7.3 g, Yield: 68%) was synthesized according to the same method as the Synthesis Example 1 of the intermediate A-29 except for using an intermediate of biphenyl boronic acid (Manufacturer: Beijing Pure Chem Co. Ltd.) instead of phenyl boronic acid.

calcd. C22H13ClN2S: C, 70.87; H, 3.51; Cl, 9.51; N, 7.51; S, 8.60. found: C, 70.81; H, 3.46; Cl, 9.50; N, 7.49; S, 8.60.

### Synthesis of Compound a-42

Compound a-42 (15.7 g, Yield: 56%) was synthesized according to the same method as the Synthesis Example 1 of the Compound 29 except for respectively using Intermediate A-a-42 and boronic ester (4) instead of the Intermediate A-29 and boronic ester (1). The elemental analysis result of the Compound a-42 was provided as follows.

calcd. C46H30N2S: C, 85.95; H, 4.70; N, 4.36; S, 4.99. found: C, 85.93; H, 4.62; N, 4.33; S, 4.98.

### Synthesis Example ad-5

### Synthesis of Compound a-46

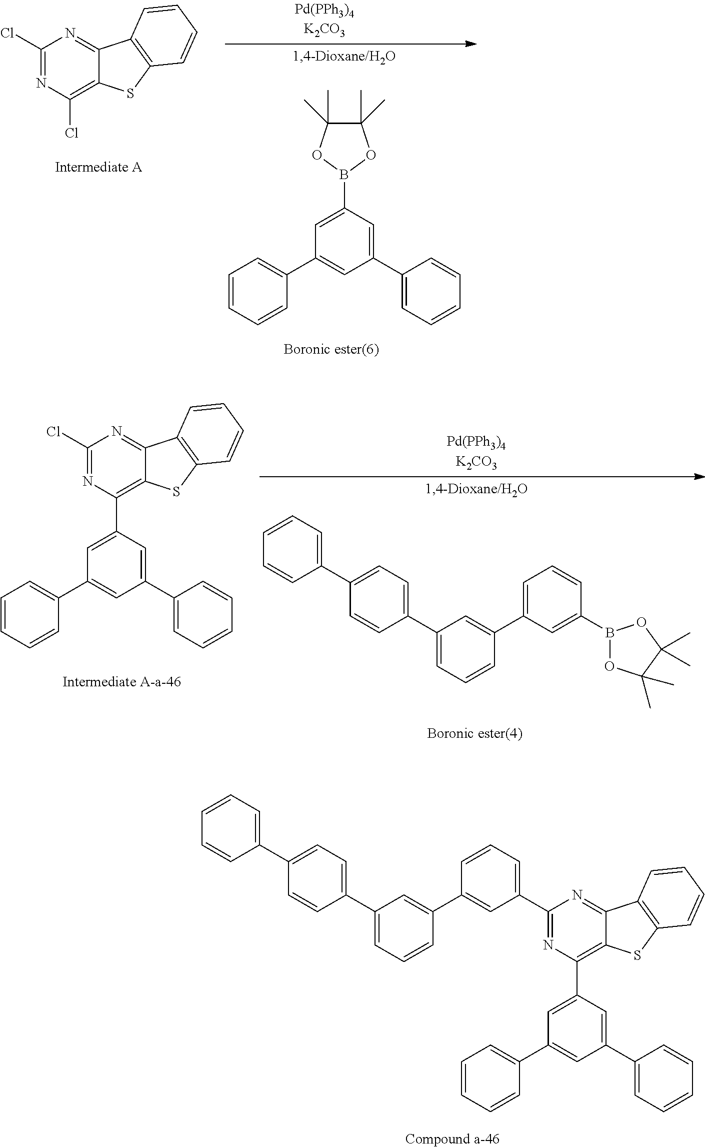

### Synthesis of Intermediate A-a-46

Intermediate A-a-46 (6.1 g, Yield: 70%) was synthesized according to the same method as the Synthesis Example 1 of the intermediate A-29 except for using an intermediate of boronic ester (6) instead of the phenyl boronic acid.

calcd. C28H17ClN2S: C, 74.91; H, 3.82; Cl, 7.90; N, 6.24; S, 7.14. found: C, 74.91; H, 3.76; Cl, 7.87; N, 6.21; S, 7.11.

### Synthesis of Compound a-46

Compound a-46 (4.4 g, Yield: 64%) was synthesized according to the same method as the Synthesis Example 1 of the Compound 29 except for respectively using the intermediate A-a-46 and an intermediate of boronic ester (4) instead of the intermediate A-29 and the intermediate of boronic ester (1). The elemental analysis result of the Compound a-46 was provided as follows.

calcd. C52H34N2S: C, 86.88; H, 4.77; N, 3.90; S, 4.46. found: C, 86.80; H, 4.73; N, 3.87; S, 4.43.

### Synthesis Example ad-6

### Synthesis of Compound a-56

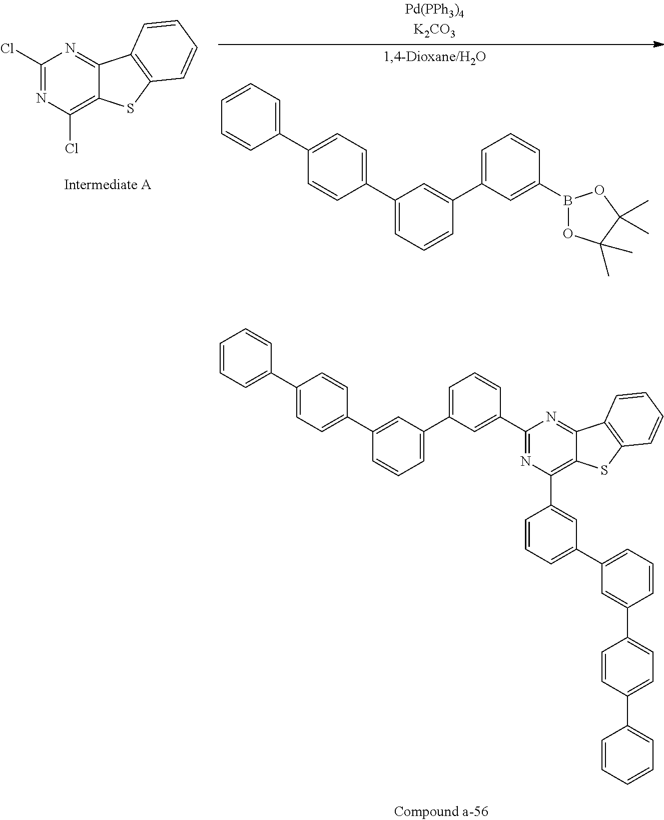 [SMILES: Clc1nc(Cl)c2sc3ccccc3c2n1 ; CC1(C)OB(c2cccc(-c3cccc(-c4ccc(-c5ccccc5)cc4)c3)c2)OC1(C)C ; c1ccc(-c2ccc(-c3cccc(-c4cccc(-c5nc(-c6cccc(-c7cccc(-c8ccc(-c9ccccc9)cc8)c7)c6)c6sc7ccccc7c6n5)c4)c3)cc2)cc1]

### Synthesis of Compound a-56

Compound a-56 (8.3 g, Yield: 74%) was synthesized according to the same method as the Synthesis Example ad-1 of the Compound a-30 except for using an intermediate of boronic ester (4) instead of the intermediate of the boronic acid (3). The elemental analysis result of the Compound a-56 was provided as follows.

calcd. C58H38N2S: C, 87.63; H, 4.82; N, 3.52; S, 4.03. found: C, 87.61; H, 4.80; N, 3.52; S, 4.02.

### Synthesis Example ad-7

### Synthesis of Compound a-70

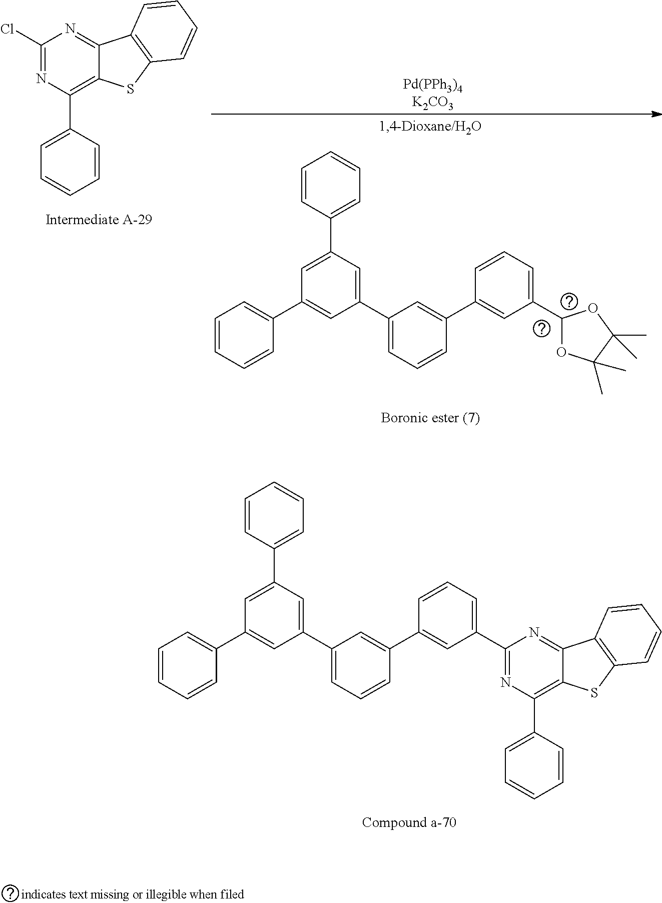 [SMILES: Clc1nc(-c2ccccc2)c2sc3ccccc3c2n1 ; CC1(C)OC(c2cccc(-c3cccc(-c4cc(-c5ccccc5)cc(-c5ccccc5)c4)c3)c2)OC1(C)C ; c1ccc(-c2cc(-c3ccccc3)cc(-c3cccc(-c4cccc(-c5nc(-c6ccccc6)c6sc7ccccc7c6n5)c4)c3)c2)cc1]

### Synthesis of Compound a-70

Compound a-70 (7.7 g, Yield: 70%) was synthesized according to the same method as the Synthesis Example of the Compound a-40 except for using boronic ester (7) instead of the boronic ester (4). The elemental analysis result of the Compound a-70 was provided as follows.

calcd. C46H30N2S: C, 85.95; H, 4.70; N, 4.36; S, 4.99. found: C, 85.90; H, 4.70; N, 4.32; S, 4.90.

### Synthesis Example ad-8

### Synthesis of Compound a-71

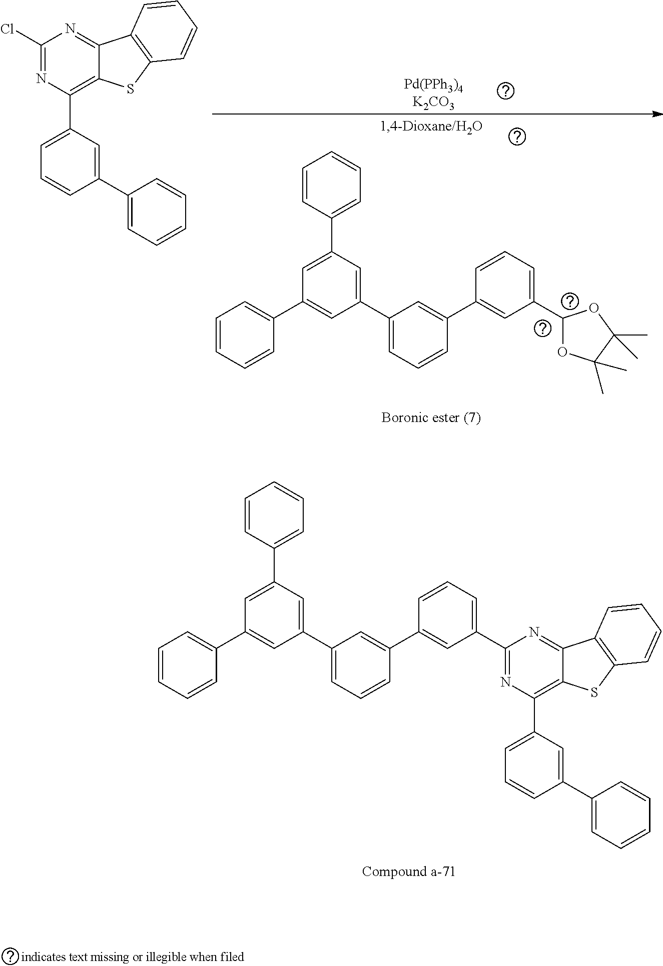 [SMILES: Clc1nc(-c2cccc(-c3ccccc3)c2)c2sc3ccccc3c2n1 ; CC1(C)OC(c2cccc(-c3cccc(-c4cc(-c5ccccc5)cc(-c5ccccc5)c4)c3)c2)OC1(C)C ; c1ccc(-c2cc(-c3ccccc3)cc(-c3cccc(-c4cccc(-c5nc(-c6cccc(-c7ccccc7)c6)c6sc7ccccc7c6n5)c4)c3)c2)cc1]

### Synthesis of Compound a-71

Compound a-71 (1.2 g, Yield: 78%) was synthesized according to the same method as the Synthesis Example of Compound a-41 except for using boronic ester (7) instead of the boronic ester (4). The elemental analysis result of the Compound a-71 as provided as follows.

calcd. C52H34N2S: C, 86.88; H, 4.77; N, 3.90; S, 4.46. found: C, 86.82; H, 4.75; N, 3.87; S, 4.42.

### Synthesis Example ad-9

### Synthesis of Compound a-74

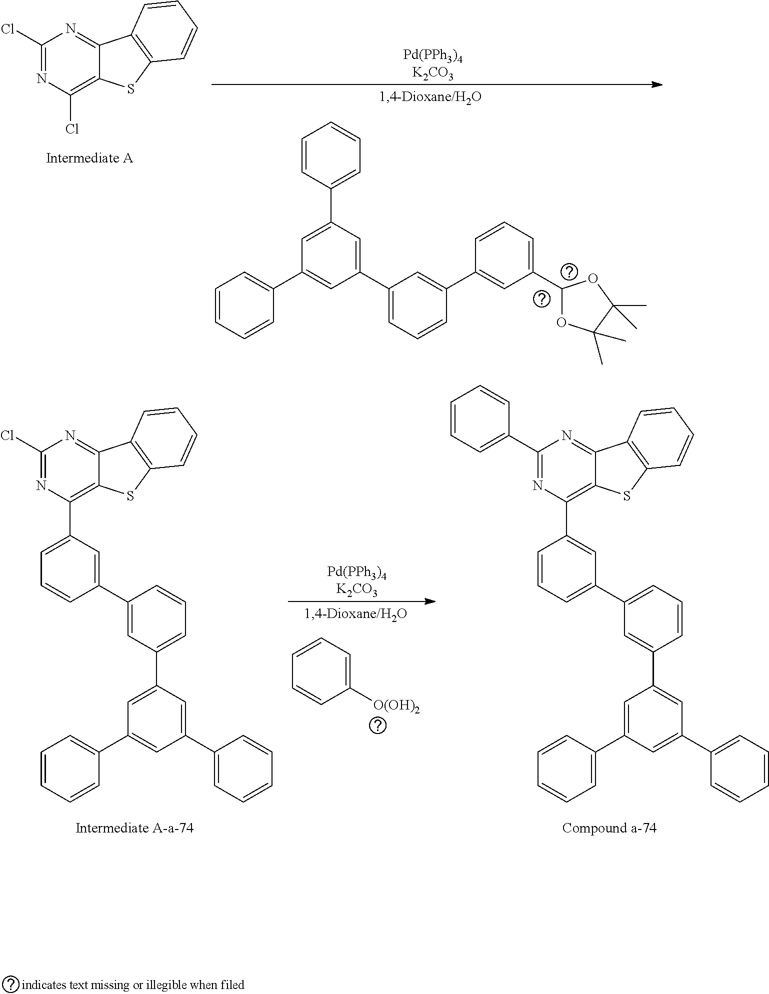

### Synthesis of Intermediate A-a-74

10.0 g (39.2 mmol) of the intermediate A, 21.9 g (43.1 mmol) of boronic ester (7), 13.5 g (98.0 mmol) of potassium carbonate, and 2.3 g (2.0 mmol) of tetrakis(triphenylphosphine)palladium (0) were added to 140 mL of dioxane and 70 mL of water in a 500 mL round flask and then heated under reflux in a nitrogen atmosphere for 16 hours at 60° C. Then, a solid crystallized by adding the mixture obtained therefrom to 150 mL of methanol was filtered, dissolved in monochlorobenzene and filtered with silica gel/Celite, and then, recrystallized with methanol after removing an appropriate amount of a solvent therefrom, obtaining Compound A-a-74 (16.5 g, Yield: 70%).

calcd. C40H25ClN2: C, 79.92; H, 4.19; Cl, 5.90; N, 4.66; S, 5.33. found: C, 79.90; H, 4.19; Cl, 5.89; N, 4.65; S, 5.31.

### Synthesis of Compound a-74

10.0 g (16.6 mmol) of the intermediate A-a-74, 2.2 g (18.3 mmol) of phenyl boronic acid, 5.8 g (41.6 mmol) of potassium carbonate, and 1.0 g (0.8 mmol) of tetrakis(triphenylphosphine)palladium (0) were added to 50 mL of dioxane and 25 mL of water in a 500 mL round flask a and then heated under reflux in a nitrogen atmosphere for 8 hours. Then, a solid crystallized by adding the mixture obtained therefrom to 150 mL of methanol was filtered, dissolved in monochlorobenzene and filtered with silica gel/Celite and then, recrystallized with methanol after removing an appropriate amount of a solvent therefrom, obtaining Compound a-74 (6.8 g, Yield: 64%). The elemental analysis result of Compound a-74 is as follows.

calcd. C46H30N2S: C, 85.95; H, 4.70; N, 4.36; S, 4.99. found: C, 85.91; H, 4.69; N, 4.33; S, 4.94.

### Synthesis Example ad-10

### Synthesis of Compound a-75

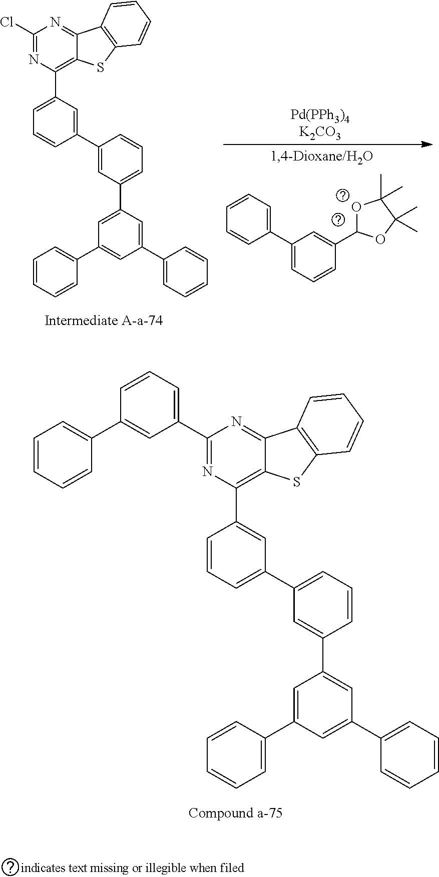

### Synthesis of Compound a-75

Compound a-75 (6.2 g, Yield: 73%) was synthesized according to the same method as the Synthesis Example ad-9 of the Compound a-74 except for using an intermediate of boronic ester (5) instead of the phenyl boronic acid. The elemental analysis result of the Compound a-75 was provided as follows.

calcd. C52H34N2S: C, 86.88; H, 4.77; N, 3.90; S, 4.46. found: C, 86.88; H, 4.73; N, 3.85; S, 4.45.

### Synthesis Example ad-11

### Synthesis of Compound a-82

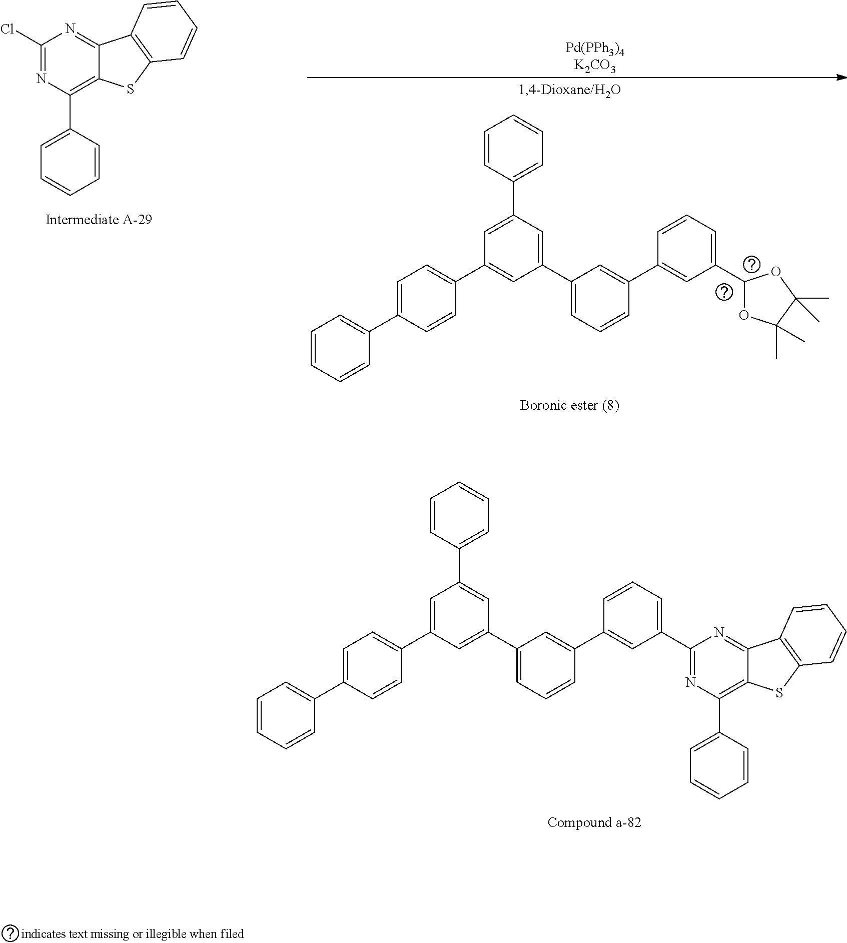 [SMILES: Clc1nc(-c2ccccc2)c2sc3ccccc3c2n1 ; CC1(C)OC(c2cccc(-c3cccc(-c4cc(-c5ccccc5)cc(-c5ccc(-c6ccccc6)cc5)c4)c3)c2)OC1(C)C ; c1ccc(-c2ccc(-c3cc(-c4ccccc4)cc(-c4cccc(-c5cccc(-c6nc(-c7ccccc7)c7sc8ccccc8c7n6)c5)c4)c3)cc2)cc1]

Compound a-82 (6.7 g, Yield: 67%) was synthesized according to the same method as the Synthesis Example ad-7 of the Compound a-70 except for using boronic ester (8) instead of the intermediate of the boronic ester (7). The elemental analysis result of the Compound a-82 was provided as follows.

calcd. C52H34N2S: C, 86.88; H, 4.77; N, 3.90; S, 4.46. found: C, 86.85; H, 4.76; N, 3.87; S, 4.46.

### Synthesis Example ad-12

### Synthesis of Compound a-84

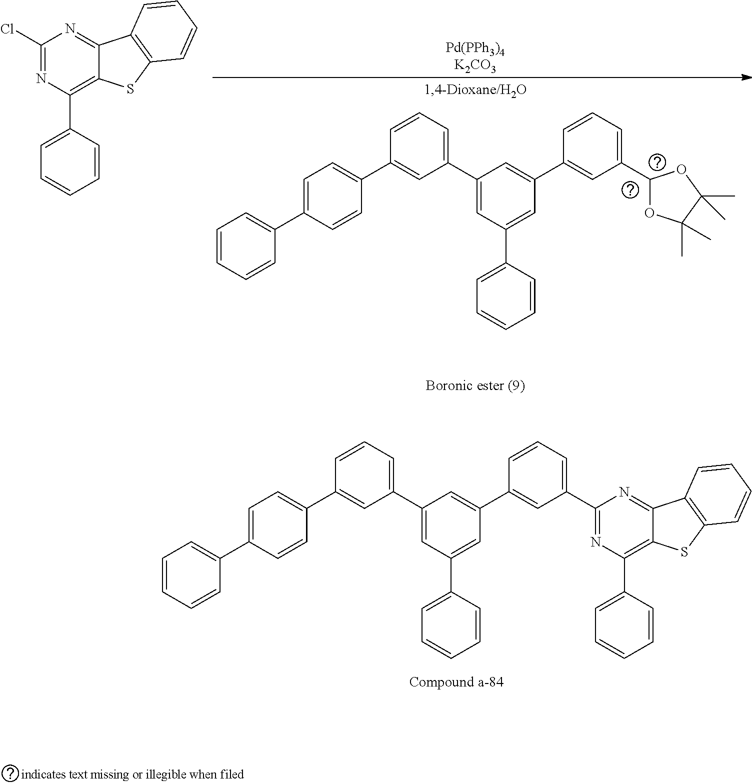 [SMILES: Clc1nc(-c2ccccc2)c2sc3ccccc3c2n1 ; CC1(C)OC(c2cccc(-c3cc(-c4ccccc4)cc(-c4cccc(-c5ccc(-c6ccccc6)cc5)c4)c3)c2)OC1(C)C ; c1ccc(-c2ccc(-c3cccc(-c4cc(-c5ccccc5)cc(-c5cccc(-c6nc(-c7ccccc7)c7sc8ccccc8c7n6)c5)c4)c3)cc2)cc1]

Compound a-84 (9.3 g, Yield: 76%) was synthesized according to the same method as the Synthesis Example ad-7 of the Compound a-70 except for using an intermediate of boronic ester (9) instead of the intermediate of the boronic ester (7). The elemental analysis result of the Compound a-84 was provided as follows.

calcd. C52H34N2S: C, 86.88; H, 4.77; N, 3.90; S, 4.46. found: C, 86.84; H, 4.77; N, 3.89; S, 4.45.

### Synthesis Example ad-13

### Synthesis of Compound a-114

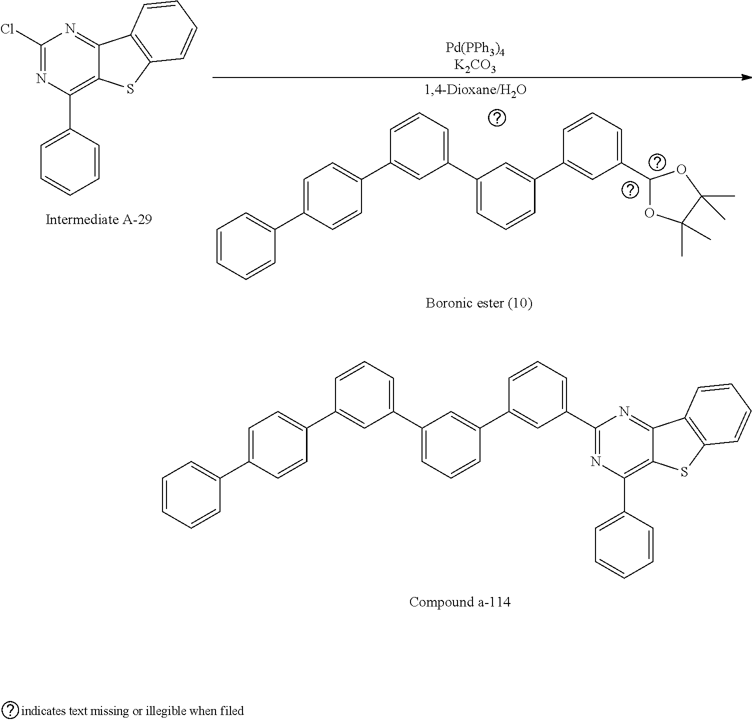 [SMILES: Clc1nc(-c2ccccc2)c2sc3ccccc3c2n1 ; CC1(C)OC(c2cccc(-c3cccc(-c4cccc(-c5ccc(-c6ccccc6)cc5)c4)c3)c2)OC1(C)C ; c1ccc(-c2ccc(-c3cccc(-c4cccc(-c5cccc(-c6nc(-c7ccccc7)c7sc8ccccc8c7n6)c5)c4)c3)cc2)cc1]

Compound a-114 (10.9 g, Yield: 75%) was synthesized according to the same method as the Synthesis Example ad-7 of the Compound a-70 except for using an intermediate of boronic ester (10) instead of the intermediate of the boronic ester (7). The elemental analysis result of the Compound a-114 was provided as follows.

calcd. C46H30N2S: C, 85.95; H, 4.70; N, 4.36; S, 4.99. found: C, 85.94; H, 4.68; N, 4.30; S, 4.87.

### Synthesis Example ad-14

### Synthesis of Compound a-108

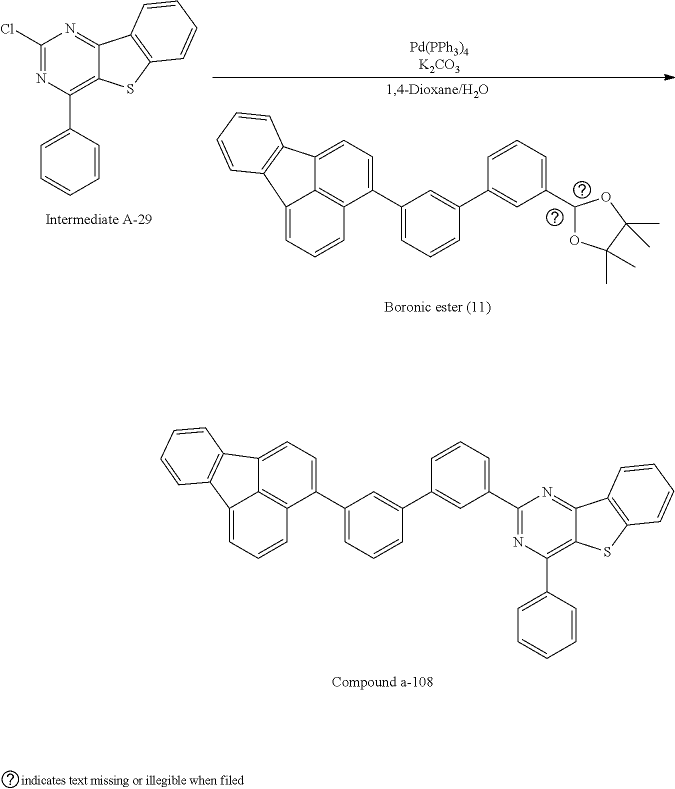 [SMILES: Clc1nc(-c2ccccc2)c2sc3ccccc3c2n1 ; CC1(C)OC(c2cccc(-c3cccc(-c4ccc5c6c(cccc46)-c4ccccc4-5)c3)c2)OC1(C)C ; c1ccc(-c2nc(-c3cccc(-c4cccc(-c5ccc6c7c(cccc57)-c5ccccc5-6)c4)c3)nc3c2sc2ccccc23)cc1]

Compound a-108 (8.4 g, Yield: 70%) was synthesized according to the same method as the Synthesis Example ad-7 of the Compound a-70 except for using an intermediate of boronic ester (11) instead of the intermediate of boronic ester (7). The elemental analysis result of the Compound a-108 was provided as follows.

calcd. C44H26N2S: C, 85.96; H, 4.26; N, 4.56; S, 5.22. found: C, 85.94; H, 4.21; N, 4.50; S, 5.22.

### Synthesis Example ad-15

### Synthesis of Compound a-110

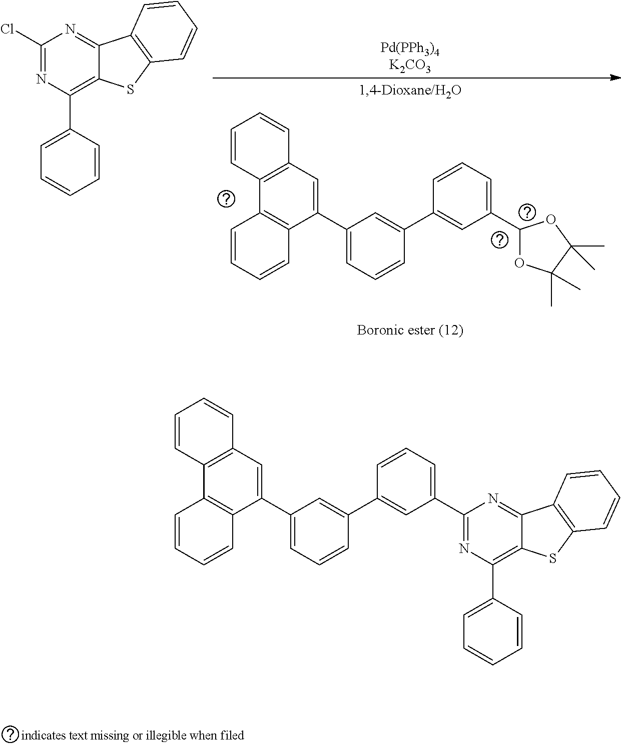 [SMILES: Clc1nc(-c2ccccc2)c2sc3ccccc3c2n1 ; CC1(C)OC(c2cccc(-c3cccc(-c4cc5ccccc5c5ccccc45)c3)c2)OC1(C)C ; c1ccc(-c2nc(-c3cccc(-c4cccc(-c5cc6ccccc6c6ccccc56)c4)c3)nc3c2sc2ccccc23)cc1]

Compound a-110 (6.7 g, Yield: 65%) was synthesized according to the same method as the Synthesis Example ad-7 of the Compound a-70 except for using an intermediate of boronic ester (12) instead of the intermediate of boronic ester (7). The elemental analysis result of the Compound a-110 was provided as follows.

calcd. C42H26N2S: C, 85.39; H, 4.44; N, 4.74; S, 5.43. found: C, 85.30; H, 4.44; N, 4.73; S, 5.42.

### Synthesis Example ad-16

### Synthesis of Compound a-112

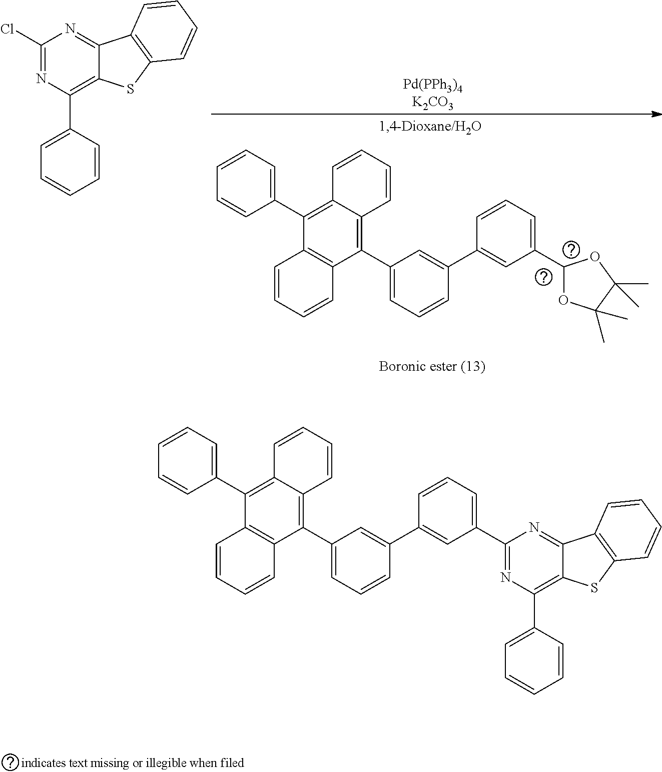 [SMILES: Clc1nc(-c2ccccc2)c2sc3ccccc3c2n1 ; CC1(C)OC(c2cccc(-c3cccc(-c4c5ccccc5c(-c5ccccc5)c5ccccc45)c3)c2)OC1(C)C ; c1ccc(-c2c3ccccc3c(-c3cccc(-c4cccc(-c5nc(-c6ccccc6)c6sc7ccccc7c6n5)c4)c3)c3ccccc23)cc1]

Compound a-112 (7.9 g, Yield: 67%) was synthesized according to the same method as the Synthesis Example ad-7 of the Compound a-70 except for using an intermediate of boronic ester (13) instead of the intermediate of boronic ester (7). The elemental analysis result of the Compound a-112 was provided as follows.

calcd. C48H30N2S: C, 86.46; H, 4.53; N, 4.20; S, 4.81. found: C, 86.45; H, 4.52; N, 4.18; S, 4.80.

### Synthesis Example ad-17

### Synthesis of Compound a-116

5.0 g (10.8 mmol) of the intermediate A-a-116, 5.0 g (10.8 mmol) of the intermediate AA-a-116, 3.7 g (53.8 mmol) of potassium carbonate, and 0.6 g (0.5 mmol) of tetrakis(triphenylphosphine)palladium (0) were added to 40 mL of dioxane and 20 mL of water in a 100 mL round flask and then heated under reflux in a nitrogen atmosphere for 12 hours. Then, a solid crystallized by adding the mixture obtained therefrom to 120 mL of methanol was filtered, dissolved in monochlorobenzene and filtered with silica gel/Celite and then, recrystallized with methanol after removing an appropriate amount of a solvent therefrom, obtaining Compound a-116 (6.1 g, Yield: 64%).

calcd. C47H30N2S: C, 86.21; H, 4.62; N, 4.28; S, 4.90. found: C, 86.21; H, 4.60; N, 4.25; S, 4.89.

### (Reference Reaction Scheme: Synthesis Scheme of Intermediate A-a-116)

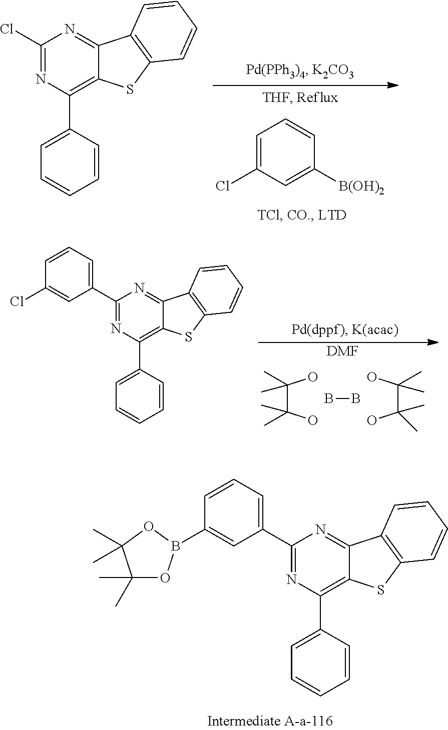

### (Reference Reaction Scheme: Synthesis Scheme of Intermediate AA-a-116)

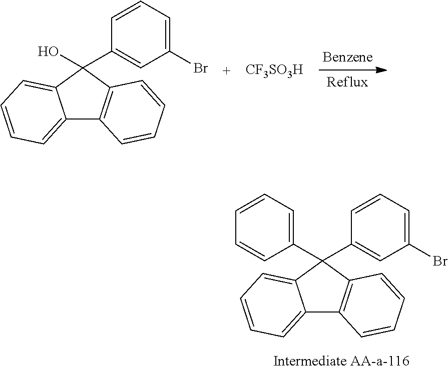

### Synthesis Example ad-18

### Synthesis of Compound b-41

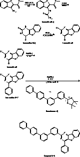

### Synthesis of Intermediate B(1) (benzo-methyl 3-ureidofuran-2-carboxylate)

Chlorosulfonylisocyanate (33.4 mL, 0.38 mol) was added in a dropwise fashion to a solution including benzo-methyl 3-aminofuran-2-carboxylate (49.0 g, 0.25 mol) in dichloromethane (1000 mL) at −78° C. in a 1000 mL round flask. The reactant was slowly heated up to room temperature and agitated for 2 hours. The agitated reactant was concentrated, Conc. HCl (100 mL) was added to its residue, and the mixture was agitated at 100° C. for one hour. The reaction mixture was cooled down to room temperature and neutralized with a saturated NaHCO₃ aqueous solution. Then, a solid produced therein was filtered, obtaining an intermediate B(1) of benzo-methyl 3-ureidofuran-2-carboxylate)(52.1 g, 87%) as a beige solid.

calcd. C₁₁H₁₀N₂O₄: C, 56.41; H, 4.30; N, 11.96; O, 27.33. found: C, 56.45; H, 4.28; N, 11.94; O, 27.32.

### Synthesis of Intermediate B (2) (benzo-furo[3,2-d]pyrimidine-2,4-diol)

The intermediate B (1) (benzo-methyl 3-ureidofuran-2-carboxylate) (50.0 g, 0.21 mol) was suspended into 1000 mL of methanol in a 2000 mL round flask, and 300 mL of 2 M NaOH was added thereto in a dropwise fashion. The reaction mixture was refluxed and agitated for 3 hours. The resultant was cooled down to room temperature and acidified into pH 3 by using Conc. HCl. After concentrating the mixture, methanol was slowly added in a dropwise fashion to the residue to precipitate a solid. The produced solid was filtered and dried, obtaining the intermediate B (2) (benzo-furo[3,2-d]pyrimidine-2,4-diol) (38.0 g, 88%).

calcd. C₁₀H₆N₂O₃: C, 59.41; H, 2.99; N, 13.86; O, 23.74. found: C, 59.41; H, 2.96; N, 13.81; O, 23.75.

### Intermediate B (benzo-2,4-dichlorofuro[3,2-d]pyrimidine)

The intermediate B (2) (benzo-furo[3,2-d]pyrimidine-2,4-diol) (37.2 g, 0.18 mol) was dissolved in phosphorous oxychloride (500 mL) in a 1000 mL round flask. The mixture was cooled down to −30° C., and N,N-diisopropylethylamine (52 mL, 0.36 mol) was slowly added thereto. The reactant was refluxed and agitated for 36 hours and then, cooled down to room temperature. The reactant was poured into ice/water an then, extracted with ethylacetate. Then, an organic layer obtained therefrom was washed with a NaHCO₃ aqueous solution and then, dried with Na₂SO₄. The obtained organic layer was concentrated, obtaining the intermediate B (benzo-2,4-dichlorofuro[3,2-d]pyrimidine) (20.4 g, 46%%).

The elemental analysis and NMR analysis results of the intermediate B are as follows.

calcd. C₁₀H₄C1₂N₂O: C, 50.24; H, 1.69; Cl, 29.66; N, 11.72; O, 6.69. found: C, 50.18; H, 1.79; Cl, 29.69; N, 11.69; O, 6.70.

300 MHz (CDCl₃, ppm): 7.55 (t, 1H), 7.71-7.82 (m, 2H), 8.25 (d, 1H)

### Synthesis of Intermediate B-37

40.0 g (167.3 mmol) of the intermediate B, 22.4 g (184.1 mmol) of phenylboronic acid, 57.8 g (418.3 mmol) of potassium carbonate, and 9.7 g (8.4 mmol) of Pd(PPh₃)₄ (tetrakis (triphenylphosphine)palladium (0)) were put in 500 mL of 1,4-dioxane and 250 mL of water in a 2000 mL flask under a nitrogen stream for 8 hours at 40° C. The mixture was added to 1500 mL of methanol, and a solid crystallized therein was filtered, dissolved in monochlorobenzene, and filtered with silica gel/Celite and then, recrystallized with methanol after removing an appropriate amount of an organic solvent therefrom, obtaining the intermediate B-37 (31.0 g, Yield: 66%).

calcd. C₁₆H₉ClN₂O: C, 68.46; H, 3.23; Cl, 12.63; N, 9.98; O, 5.70. found: C, 68.95; H, 3.08; Cl, 12.17; N, 10.01; O, 5.62.

### Synthesis of Compound b-41

10.2 g (36.5 mmol) of the intermediate B-37, 8.5 g (19.6 mmol) of boronic ester (4), 6.2 g (44.5 mmol) of potassium carbonate, and 1.0 g (0.9 mmol) of tetrakis(triphenylphosphine) palladium (0) were added to 60 mL of 1,4-dioxane and 30 mL of water in a 500 mL round flask and then heated under reflux in a nitrogen atmosphere for 12 hours. The mixture was added to 200 mL of methanol, and a solid crystallized therein was filtered, dissolved in monochlorobenzene, and filtered with silica gel/Celite and then, recrystallized with methanol after removing an appropriate amount of an organic solvent therefrom, obtaining the compound b-41 (7.0 g, Yield: 71%). The elemental analysis result of the compound b-41 is as follows.

calcd. C₄₀H₂₆N₂O: C, 87.25; H, 4.76; N, 5.09; O, 2.91. found: C, 87.22; H, 4.71; N, 5.08; O, 2.90.

### Synthesis Example ad-19

### Synthesis of Compound b-71

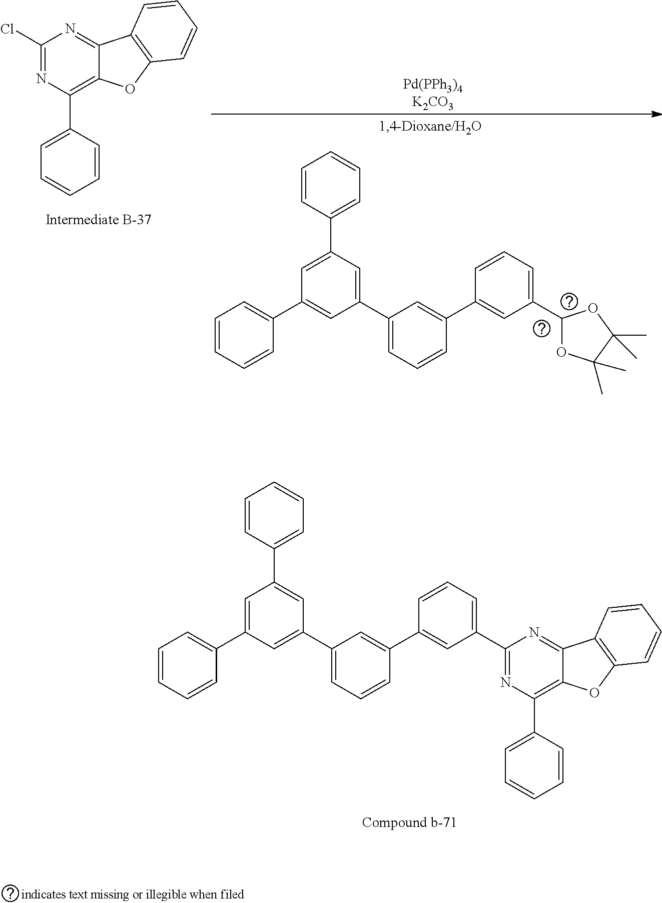 [SMILES: Clc1nc(-c2ccccc2)c2oc3ccccc3c2n1 ; CC1(C)OC(c2cccc(-c3cccc(-c4cc(-c5ccccc5)cc(-c5ccccc5)c4)c3)c2)OC1(C)C ; c1ccc(-c2cc(-c3ccccc3)cc(-c3cccc(-c4cccc(-c5nc(-c6ccccc6)c6oc7ccccc7c6n5)c4)c3)c2)cc1]

5.0 g (17.8 mmol) of the intermediate B-37, 10.0 g (19.6 mmol) of boronic ester (7), 6.2 g (44.5 mmol) of potassium carbonate, and 1.0 g (0.9 mmol) of tetrakis(triphenylphosphine) palladium (0) were added to 60 mL of 1,4-dioxane and 30 mL of water in a 500 mL round flask and then heated under reflux in a nitrogen atmosphere for 12 hours. The mixture was added to 200 mL of methanol, and a solid crystallized therein was filtered, dissolved in monochlorobenzene, and filtered with silica gel/Celite and then, recrystallized with methanol after removing an appropriate amount of an organic solvent therefrom, obtaining the compound b-71 (7.5 g, Yield: 67%). The elemental analysis result of the compound b-71 is as follows.

calcd. C₄₆H₃₀N₂O: C, 88.15; H, 4.82; N, 4.47; O, 2.55. found: C, 88.11; H, 4.81; N, 4.43; O, 2.52.

### Synthesis Example ad-20

### Synthesis of Compound b-116

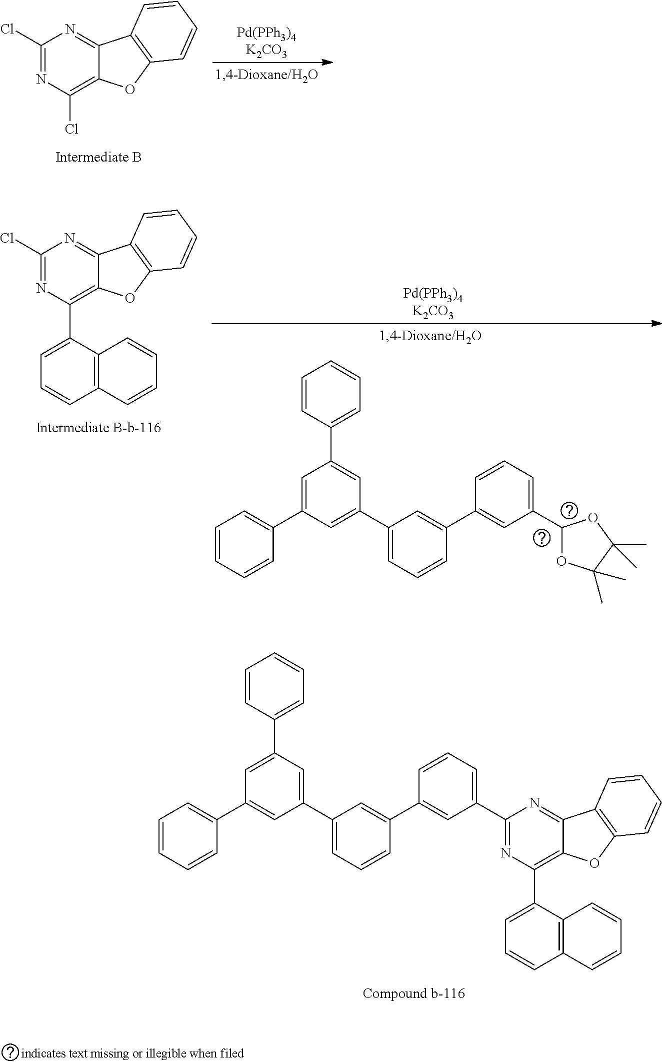 [SMILES: Clc1nc(Cl)c2oc3ccccc3c2n1 ; Clc1nc(-c2cccc3ccccc23)c2oc3ccccc3c2n1 ; CC1(C)OC(c2cccc(-c3cccc(-c4cc(-c5ccccc5)cc(-c5ccccc5)c4)c3)c2)OC1(C)C ; c1ccc(-c2cc(-c3ccccc3)cc(-c3cccc(-c4cccc(-c5nc(-c6cccc7ccccc67)c6oc7ccccc7c6n5)c4)c3)c2)cc1]

### Synthesis of Intermediate B-b-116

30.0 g (125.5 mmol) of the intermediate B, 23.7 g (138.0 mmol) of naphthalen-1-yl boronic acid, 43.4 g (313.7 mmol) of potassium carbonate, and 7.3 g (6.3 mmol) of tetrakis(triphenylphosphine) palladium (0) were added to 400 mL of 1,4-dioxane and 200 mL of water in a 1000 mL flask and then, heated at 55° C. under a nitrogen stream for 16 hours. The obtained mixture was added to 1200 mL of methanol, and a solid crystallized therein was filtered, dissolved in monochlorobenzene, and filtered with silica gel/Celite and then, recrystallized with methanol after removing an appropriate amount of an organic solvent therefrom, obtaining the intermediate B-b-116 (29.1 g, Yield: 70%).

calcd. C20H11ClN2O: C, 72.62; H, 3.35; Cl, 10.72; N, 8.47; O, 4.84. found: C, 72.60; H, 3.35; Cl, 10.71; N, 8.40; O, 4.83.

### Synthesis of Compound b-116

5.0 g (15.1 mmol) of the intermediate B-b-116, 8.5 g (16.6 mmol) of boronic ester (7), 5.2 g (37.8 mmol) of potassium carbonate, and 0.9 g (0.8 mmol) of tetrakis(triphenylphosphine) palladium (0) were added to 50 mL of 1,4-dioxane and 25 mL of water in a 250 mL round flask and then heated under reflux in a nitrogen atmosphere for 12 hours. The obtained mixture was added to 150 mL of methanol, and a solid crystallized therein was filtered, dissolved in monochlorobenzene, and filtered with silica gel/Celite, and then, recrystallized with methanol after removing an appropriate amount of an organic solvent, obtaining the compound b-116 (7.1 g, Yield: 69%). The elemental analysis result of the compound b-116 is as follows.

calcd. C₅₀H₃₂N₂O: C, 88.73; H, 4.77; N, 4.14; O, 2.36. found: C, 88.70; H, 4.76; N, 4.07; O, 2.19.

### Synthesis Example ad-21

### Synthesis of Intermediate C

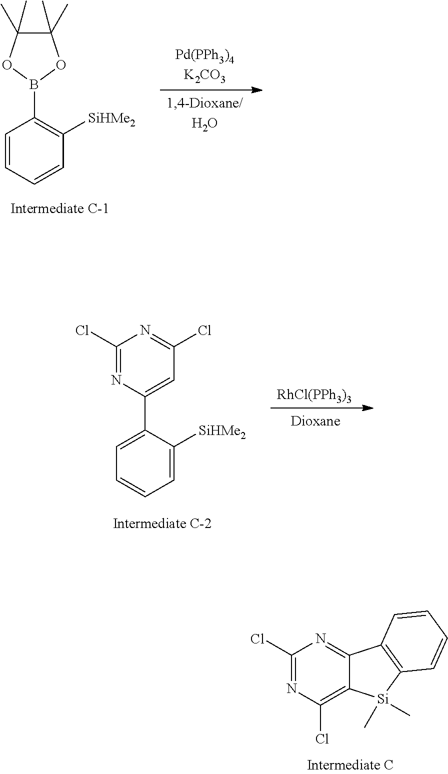

### Synthesis of Intermediate C-2

45.0 g (171.7 mmol) of the intermediate C-1, 30.0 g (163.5 mmol) of 2,4,6-trichloropyrimidine, 56.5 g (408.9 mmol) of potassium carbonate, and 9.5 g (8.2 mmol) of tetrakis (triphenylphosphine)palladium were added to 540 mL of 1,4-dioxane and 270 mL of water in a 2000 mL flask and then heated under reflux in a nitrogen atmosphere for 12 hours. The obtained mixture was added to 1000 mL of methanol, and a solid crystallized therein was filtered, dissolved in toluene and filtered with silica gel/Celite and then, recrystallized with methanol after removing an appropriate amount of an organic solvent, obtaining the intermediate C-2 (37.0 g, Yield: 76%).

calcd. C12H12Cl2N2Si: C, 50.89; H, 4.27; Cl, 25.03; N, 9.89; Si, 9.92. found: C, 50.32; H, 4.22; Cl, 24.98; N, 9.73; Si, 9.84.

### Synthesis of Intermediate C

37.0 g (130.6 mmol) of the intermediate C-2 and 2.4 g (2.6 mmol) of chlorotris(triphenylphosphine)rhodium (I) were put in a 1000 mL flask, 600 mL of 1,4-dioxane was added thereto in a dropwise fashion, and the mixture was heated under reflux in a nitrogen atmosphere for 8 hours. When the reaction was complete, a residue obtained after removing an organic layer was treated through column chromatography, obtaining the intermediate C (20.2 g, Yield: 55%).

calcd. C12H10Cl2N2Si: C, 51.25; H, 3.58; Cl, 25.21; N, 9.96; Si, 9.99. found: C, 51.15; H, 3.53; Cl, 25.16; N, 9.90; Si, 9.93.

### Synthesis Example ad-22

### Synthesis of Compound c-40

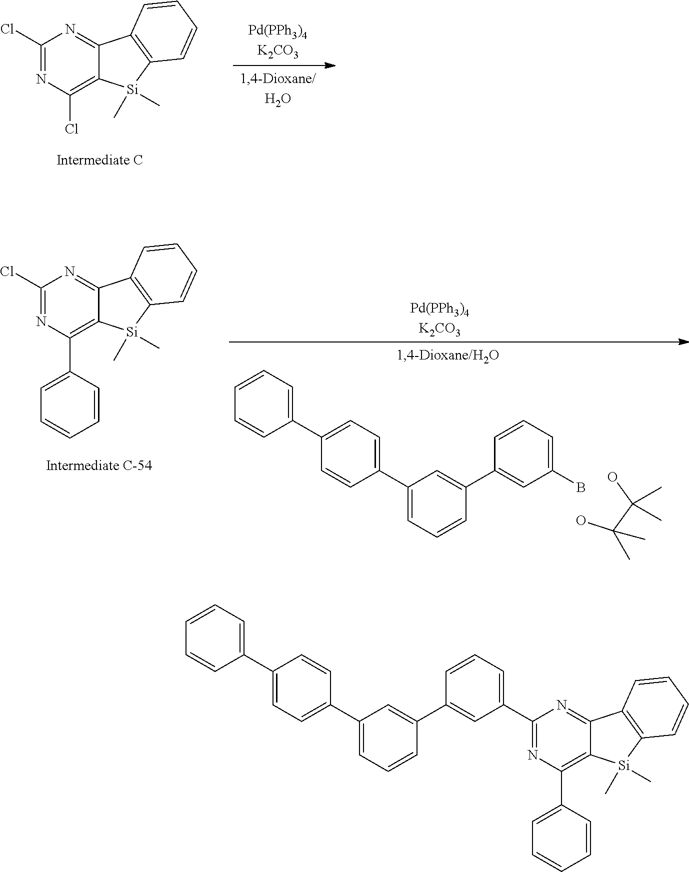

### Synthesis of Intermediate C-54

20.0 g (71.1 mmol) of the intermediate C, 9.5 g (78.2 mmol) of phenylboronic acid, 24.6 g (177.8 mmol) of potassium carbonate, and 4.1 g (3.6 mmol) of tetrakis(triphenylphosphine) palladium (0) were added to 200 mL of 1,4-dioxane and 100 mL of water in a 500 mL flask and then, heated at 55° C. under a nitrogen stream for 16 hours. The obtained mixture was added to 600 mL of methanol, and a solid crystallized therein was filtered, dissolved in monochlorobenzene, and filtered with silica gel/Celite and then, recrystallized with methanol after removing an appropriate amount of an organic solvent, obtaining the intermediate C-54 (17.2 g, Yield: 75%).

calcd. C18H15ClN2Si: C, 66.96; H, 4.68; Cl, 10.98; N, 8.68; Si, 8.70. found: C, 66.92; H, 4.63; Cl, 10.96; N, 8.67; Si, 8.65.

### Synthesis of Compound c-40

5.0 g (15.5 mmol) of the intermediate C-54, 7.4 g (17.0 mmol) of boronic ester (4), 5.4 g (38.7 mmol) of potassium carbonate, and 0.9 g (0.8 mmol) of tetrakis(triphenylphosphine) palladium (0) were added to 40 mL of 1,4-dioxane and 20 mL of water in a 100 mL round flask and then heated under reflux in a nitrogen atmosphere for 8 hours. The obtained mixture was added to 120 mL of methanol, and a solid crystallized therein was filtered, dissolved in monochlorobenzene, and filtered with silica gel/Celite and then, recrystallized with methanol after removing an appropriate amount of an organic solvent, obtaining the compound c-40 (6.5 g, Yield: 71%). The elemental analysis result of the compound c-40 is as follows.

calcd. C42H32N2Si: C, 85.10; H, 5.44; N, 4.73; Si, 4.74. found: C, 85.07; H, 5.42; N, 4.70; Si, 4.74.

### Synthesis Example ad-23

### Synthesis of Compound c-70

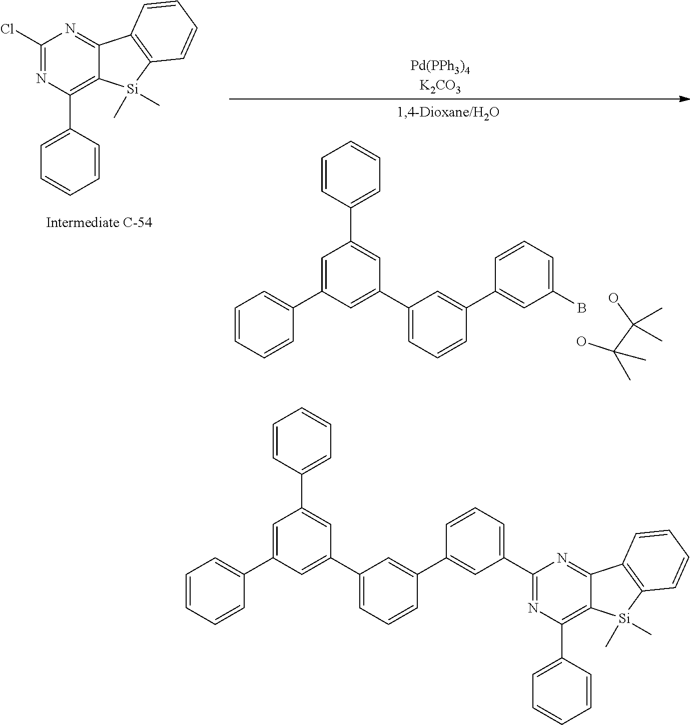

A compound c-70 (7.1 g, Yield: 69%) was synthesized according to the same method as the Synthesis Example ad-22 of the compound c-40 except for using boronic ester (7) instead of the boronic ester (4). The elemental analysis result of the compound c-70 is as follows.

calcd. C48H36N2Si: C, 86.19; H, 5.42; N, 4.19; Si, 4.20. found: C, 86.18; H, 5.40; N, 4.16; Si, 4.16.

### Synthesis Example ad-24

### Synthesis of Compound d-119

A compound d-119 provided as specific examples of a compound of the present invention was synthesized through the following four steps.

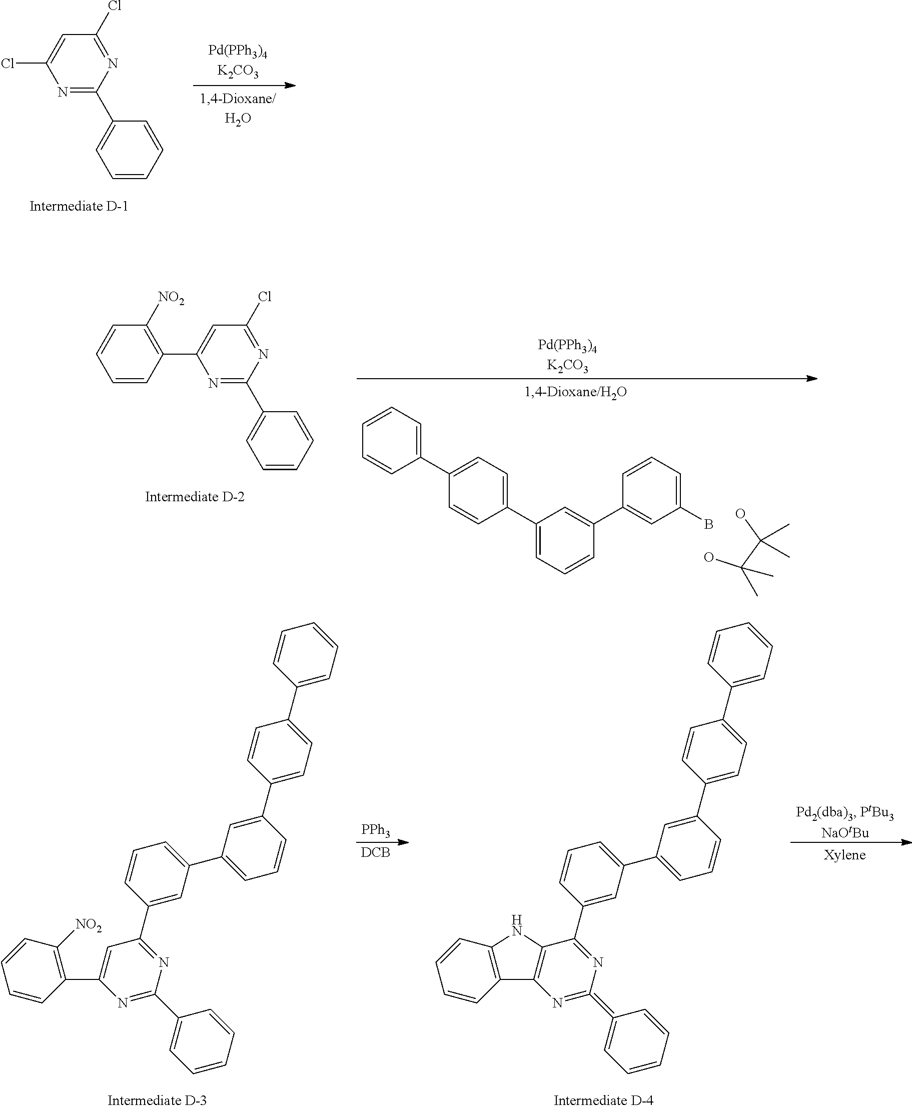

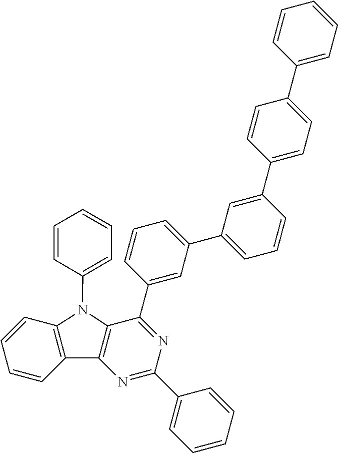 [SMILES: c1ccc(-c2ccc(-c3cccc(-c4cccc(-c5nc(-c6ccccc6)nc6c7ccccc7n(-c7ccccc7)c56)c4)c3)cc2)cc1]

### Synthesis of Intermediate D-2

50.0 g (222.2 mmol) of the intermediate D-1 (Manufacturer: TCI Inc.), 50.1 g (233.3 mmol) of 4,4,5,5-tetramethyl-2-(2-nitrophenyl)-1,3,2-dioxaborane, 76.8 g (555.4 mmol) of potassium carbonate, and 12.8 g (11.1 mmol) of tetrakis (triphenylphosphine)palladium were added to 700 mL of 1,4-dioxane and 350 mL of water in a 2000 mL flask and then heated under reflux in a nitrogen atmosphere for 12 hours. The obtained mixture was added to 2000 mL of methanol, and a solid crystallized therein was filtered, dissolved in toluene, and filtered with silica gel/Celite and then, recrystallized with methanol after removing an appropriate amount of an organic solvent, obtaining the intermediate D-2 (54.5 g, Yield: 75%).

calcd. C16H10ClN3O2: C, 61.65; H, 3.23; C1, 11.37; N, 13.48; O, 10.27. found: C, 61.23; H, 3.15; Cl, 11.37; N, 13.21; O, 10.20.

### Synthesis of Intermediate D-3

20.0 g (64.2 mmol) of the intermediate D-2, 29.1 g (67.4 mmol) of boronic ester (4), 22.2 g (160.4 mmol) of potassium carbonate, and 3.7 g (3.2 mmol)tetrakis (triphenylphosphine)palladium were added to 200 mL of 1,4-dioxane and 100 mL of water in a 500 mL flask and then heated under reflux in a nitrogen atmosphere for 12 hours. The obtained mixture was added to 600 mL of methanol, and a solid crystallized therein was filtered, dissolved in toluene, and filtered with silica gel/Celite and then, recrystallized with methanol after removing an appropriate amount of an organic solvent, obtaining the intermediate D-3 (23.9 g, Yield: 61%).

calcd. C₄₀H₂₇N₃C₂: C, 82.60; H, 4.68; N, 7.22; O, 5.50. found: C, 82.60; H, 4.63; N, 7.21; O, 5.49.

### Synthesis of Intermediate D-4

The intermediate D-3 (20.0 g, 34.4 mmol) and PPh₃ (27.1 g, 103.2 mmol) were out in a 250 mL flask, 80 mL of 1,2-dichlorobenzene (DCB) was added thereto, and the mixture was agitated at 150° C. for 12 hours after exchanged with nitrogen. The resultant was cooled down to room temperature after distilling and removing DCB, dissolved in a small amount of toluene, and purified through column chromatography (hexane), obtaining the intermediate D-4 (10.3 g, Yield: 54%).

calcd. C40H27N3: C, 87.40; H, 4.95; N, 7.64. found: C, 87.40; H, 4.93; N, 7.59.

### Synthesis of Compound d-119

10.0 g (27.3 mmol) of the intermediate D-4, 4.5 g (28.6 mmol) of bromobenzene, 5.2 g (54.5 mmol) of sodium t-butoxide, 1.6 g (2.7 mmol) of Pd(dba)₂, and 2.2 mL of tri t-butylphosphine (50% in toluene) were added to 180 mL of xylene in a 500 mL round flask and then heated under reflux in a nitrogen atmosphere for 15 hours. The obtained mixture was added to 360 mL of methanol, and a solid crystallized therein was filtered, dissolved in monochlorobenzene, and filtered with silica gel/Celite and then, recrystallized with methanol after removing an appropriate amount of an organic solvent, obtaining the compound d-40 (11.8 g, Yield: 69%). The elemental analysis result of the compound d-119 is as follows.

calcd. C46H31N3: C, 88.29; H, 4.99; N, 6.72. found: C, 88.20; H, 4.95; N, 6.71.

### Synthesis Example ad-25

### Synthesis of Compound e-70

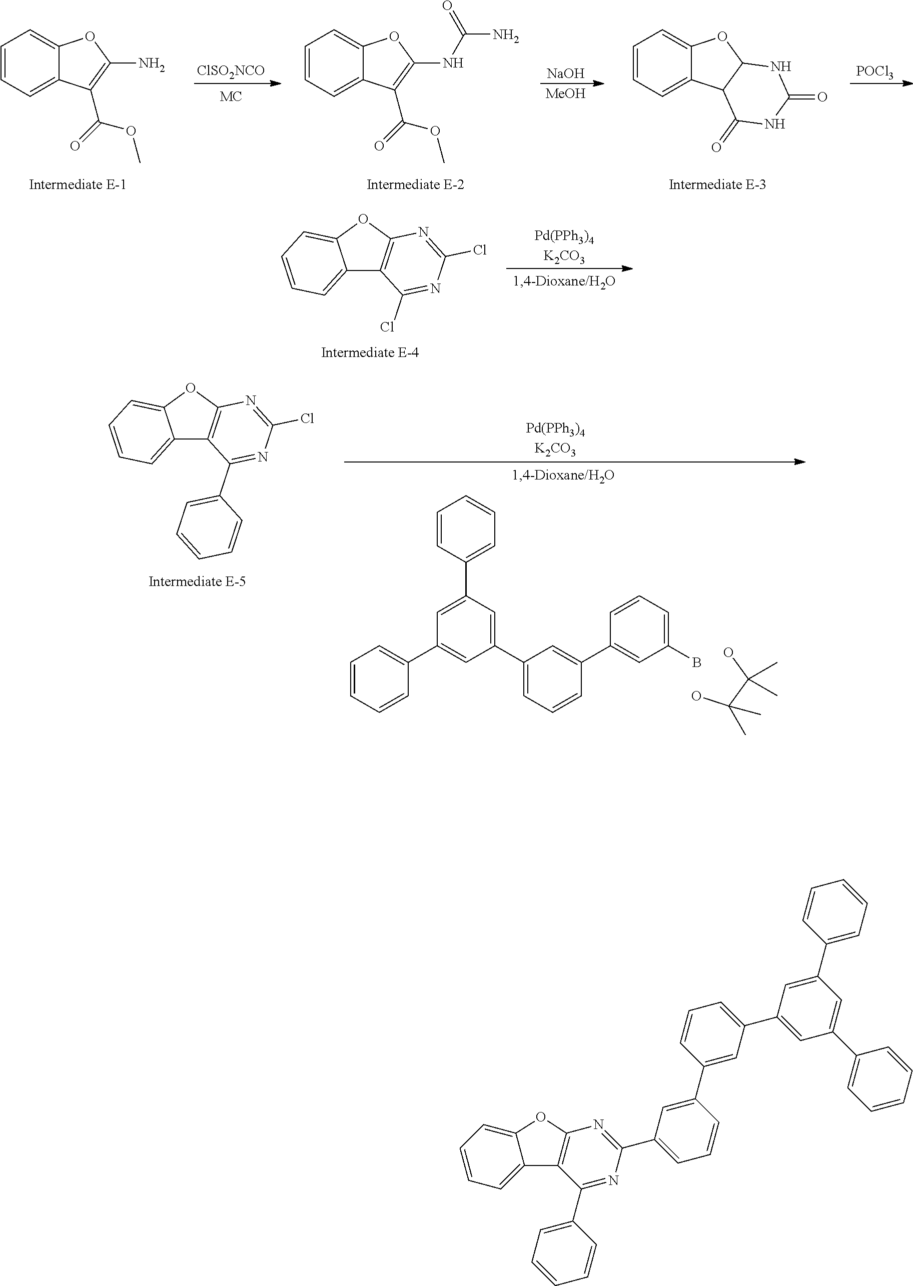

### Synthesis of Intermediate E-2

Chlorosulfonylisocyanate (23.7 mL, 274.6 mmol) was added in a dropwise fashion to a solution including an intermediate E-1 (35.0 g, 183.1 mmol) in dichloromethane (1000 mL) in a 2000 mL round flask at −78° C. The reactant was slowly heated to room temperature and agitated for 2 hours. After concentrating the reactant, 6N (300 mL) was added to the residue, and the mixture was agitated at 100° C. for 1 hour. The reaction mixture was cooled down to room temperature and neutralized with a saturated NaHCO₃ aqueous solution. Then, a solid produced therein was filtered, obtaining the intermediate E-2 (43.2 g, Yield: 88%) as a beige solid.

calcd. C10H9NO3: C, 62.82; H, 4.74; N, 7.33; O, 25.11. found: C, 62.82; H, 4.74; N, 7.33; O, 25.11.

### (Reference Reaction Scheme: Synthesis Scheme of Intermediate E-1)

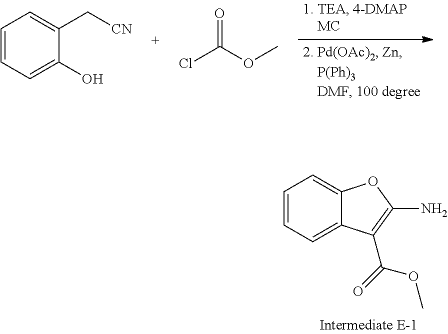

### Synthesis of Intermediate E-3

The intermediate E-2 (40.0 g, 0.19 mol) was suspended in 1000 mL of methanol in a 1000 mL round flask, and 300 mL of 2 M NaOH was added thereto in a dropwise fashion. The reaction mixture was refluxed and agitated for 3 hours. The resultant was cooled down to room temperature and acidified into pH 3 by using Conc. HCl. After concentrating the mixture, methanol was slowly added to the residue in a dropwise fashion to precipitate a solid. The solid was filtered and dried, obtaining the intermediate E-3 (39.0 g, Yield: 85%).

calcd. C11H10N2O4: C, 56.41; H, 4.30; N, 11.96; O, 27.33. found: C, 56.40; H, 4.20; N, 11.92; O, 27.31.

### Synthesis of Intermediate E-4

A mixture of the intermediate E-3 (39.0 g, 191.0 mmol) and 200 mL of phosphorous oxychloride was refluxed and agitated for 8 hours in a 500 mL round flask. The reaction mixture was cooled down to room temperature and then, poured into ice/water while strongly agitated to perform a precipitation. The obtained reactant was filtered, obtaining the intermediate E-4. (40.7 g, Yield: 89%, a white solid)

calcd. C10H4C12N2O: C, 50.24; H, 1.69; Cl, 29.66; N, 11.72; O, 6.69. found: C, 50.21; H, 1.65; Cl, 29.63; N, 11.64; O, 6.62.

### Synthesis of Intermediate E-5

10.0 g (41.8 mmol) of the intermediate E-4, 5.4 g (43.9 mmol) of phenylboronic acid, 14.5 g (104.6 mmol) of potassium carbonate, and 2.4 g (2.1 mmol) of tetrakis(triphenylphosphine) palladium (0) were added to 140 mL of 1,4-dioxane and 70 mL of water in a 500 mL flask and then, heated at 60° C. under a nitrogen stream for 10 hours. The obtained mixture was added to 450 mL of methanol, and a solid crystallized therein was filtered, dissolved in monochlorobenzene and filtered with silica gel/Celite and then, recrystallized with methanol after removing an appropriate amount of an organic solvent, obtaining the intermediate E-5 (8.0 g, Yield: 65%).

calcd. C16H9ClN2O: C, 68.46; H, 3.23; Cl, 12.63; N, 9.98; O, 5.70. found: C, 68.40; H, 3.22; Cl, 12.61; N, 9.94; O, 5.70.

### Synthesis of Compound e-70

5.0 g (17.8 mmol) of the intermediate E-5, 9.5 (18.7 mmol) of boronic ester (7), 6.2 g (44.5 mmol) of potassium carbonate, and 1.0 g (0.9 mmol) of tetrakis(triphenylphosphine) palladium (0) were added to 60 mL of 1,4-dioxane and 30 mL of water in a 250 mL round flask and then heated under reflux in a nitrogen atmosphere for 12 hours. The obtained mixture was added to 200 mL of methanol, and a solid crystallized therein was filtered, dissolved in monochlorobenzene, and filtered with silica gel/Celite and then, recrystallized with methanol after removing an appropriate amount of an organic solvent, obtaining the compound e-70 (8.1 g, Yield: 67%). The elemental analysis result of the compound e-70 is as follows.

calcd. C46H30N2O: C, 88.15; H, 4.82; N, 4.47; O, 2.55. found: C, 88.14; H, 4.80; N, 4.39; O, 2.53.

### Synthesis Example ad-26

### Synthesis of Compound f-70

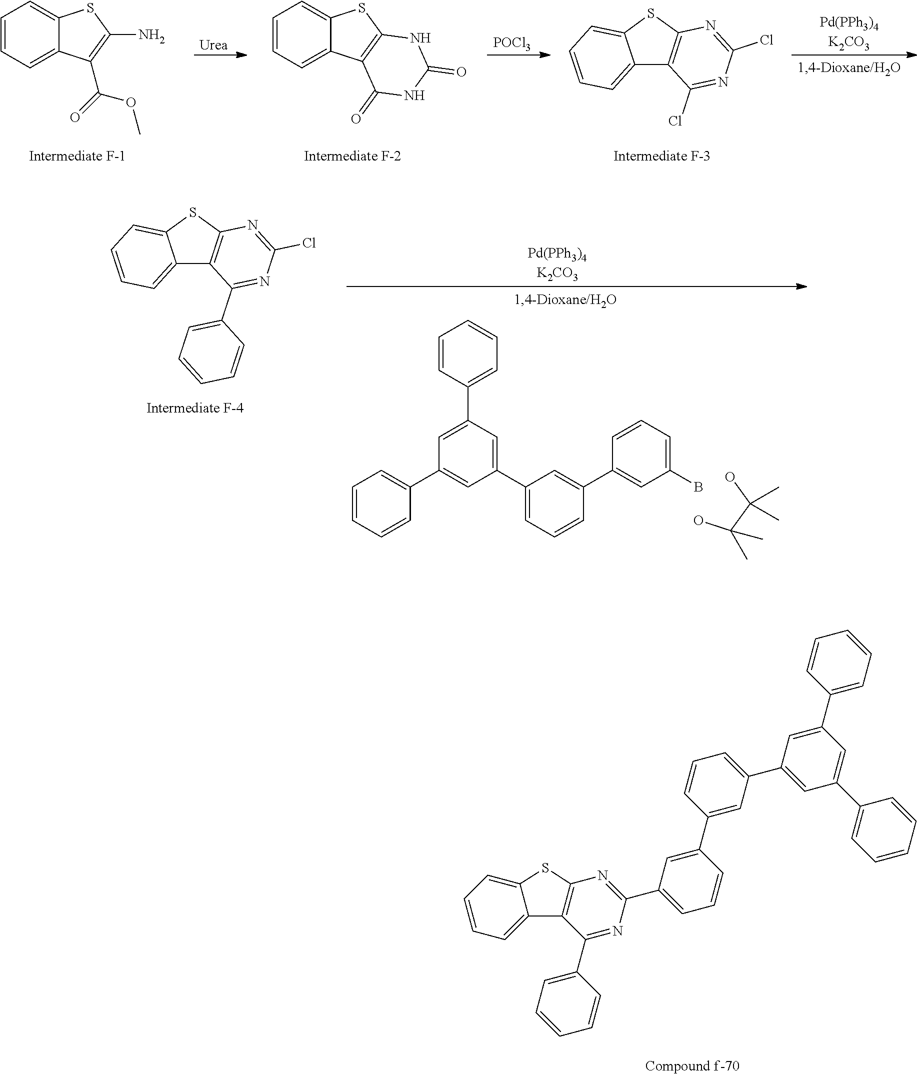

### Synthesis of Intermediate F-2

A mixture of the intermediate F-1 (35.0 g, 0.17 mol) and urea (50.7 g, 0.84 mol) was agitated at 200° C. for 2 hours in a 250 mL round flask. The high temperature reaction mixture was cooled down to room temperature and poured into a sodium hydroxide solution, the mixture was filtered to remove impurities and then, acidified (HCl, 2N), and a precipitate obtained therefrom was dried, obtaining the intermediate F-2 (18.9 g, Yield: 51%).

calcd. C10H6N2O2S: C, 55.04; H, 2.77; N, 12.84; O, 14.66; S, 14.69. found: C, 55.01; H, 2.77; N, 12.83; O, 14.65; S, 14.63.

### (Reference Reaction Scheme: Synthesis Reaction Scheme of Intermediate F-1)

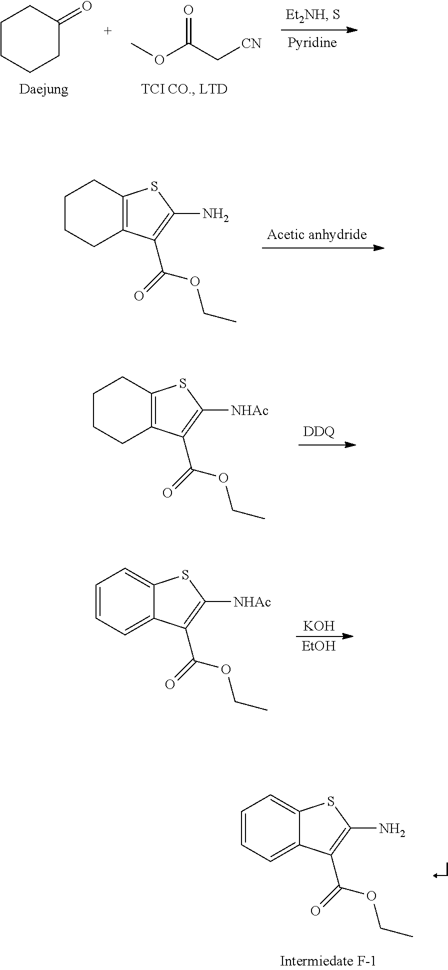

### Synthesis of Intermediate F-3

100 mL of a mixture of the intermediate F-2 (18.9 g, 99.2 mmol) and phosphorous oxychloride was refluxed and agitated for 6 hours in a 250 mL round flask. The reaction mixture was cooled down to room temperature and then, poured into ice/water while strongly agitated to produce a precipitate. The obtained reactant was filtered, obtaining the intermediate F-3. (17.5 g, Yield: 85%, a white solid)

calcd. C10H4C12N2S: C, 47.08; H, 1.58; Cl, 27.79; N, 10.98; S, 12.57. found: C, 47.04; H, 1.53; Cl, 27.74; N, 10.96; S, 12.44.

### Synthesis of Intermediate F-4

10.0 g (39.2 mmol) of the intermediate F-3, 5.3 g (43.1 mmol) of phenylboronic acid, 13.5 g (98.0 mmol) of potassium carbonate, and 2.3 g (2.0 mmol) of tetrakis(triphenylphosphine) palladium (0) were added to 140 mL of 1,4-dioxane and 70 mL of water in a 500 mL flask and then, heated at 60° C. under a nitrogen stream for 10 hours. The obtained mixture was added to 450 mL of methanol, and a solid crystallized therein was filtered, dissolved in monochlorobenzene and filtered with silica gel/Celite and then, recrystallized with methanol after removing an appropriate amount of an organic solvent, obtaining the intermediate F-4 (8.0 g, Yield: 69%).

calcd. C16H9ClN2S: C, 64.75; H, 3.06; Cl, 11.95; N, 9.44; S, 10.80. found: C, 64.72; H, 3.06; Cl, 11.94; N, 9.42; S, 10.77.

### Synthesis of Compound f-70

5.0 g (16.9 mmol) of the intermediate F-4, 9.4 g (18.5 mmol) of boronic ester (7), 5.8 g (42.1 mmol) of potassium carbonate, and 1.0 g (0.8 mmol) of tetrakis(triphenylphosphine) palladium (0) were added to 60 mL of 1,4-dioxane and 30 mL of water in a 250 mL round flask and then heated under reflux in a nitrogen atmosphere for 12 hours. The obtained mixture was added to 200 mL of methanol, and a solid crystallized therein was filtered, dissolved in monochlorobenzene and filtered with silica gel/Celite and then, recrystallized with methanol after removing an appropriate amount of an organic solvent, obtaining the compound f-70 (7.9 g, Yield: 73%). The elemental analysis result of the compound f-70 is as follows.

calcd. C46H30N2O: C, 88.15; H, 4.82; N, 4.47; O, 2.55. found: C, 88.12; H, 4.76; N, 4.44; O, 2.52.

### Synthesis Example ad-27

### Synthesis of Compound e-71

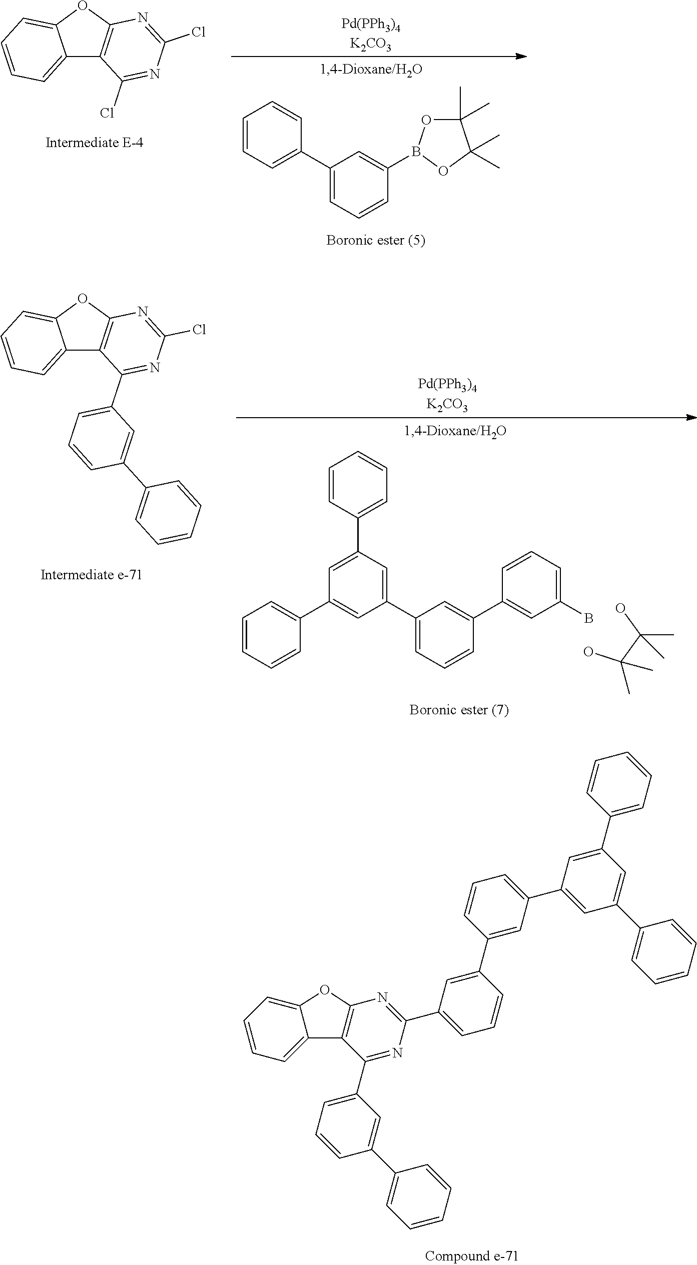

### Synthesis of Intermediate e-71

An intermediate e-71 (8.1 g, Yield: 70%) was synthesized according to the same method as the Synthesis Example ad-25 of the intermediate E-5 except for using boronic ester (5) instead of the phenylboronic acid.

calcd. C22H13ClN20: C, 74.06; H, 3.67; Cl, 9.94; N, 7.85; O, 4.48. found: C, 74.01; H, 3.65; Cl, 9.89; N, 7.84; O, 4.42.

### Synthesis of Compound e-71

A compound e-71 (7.5 g, Yield: 72%) was synthesized according to the same method as the Synthesis Example ad-25 of the compound e-70 except for using the intermediate e-71 instead of the intermediate E-5.

calcd. C52H34N2O: C, 88.86; H, 4.88; N, 3.99; O, 2.28. found: C, 88.81; H, 4.87; N, 3.96; O, 2.23.

### Synthesis Example ad-28

### Synthesis of Compound e-74

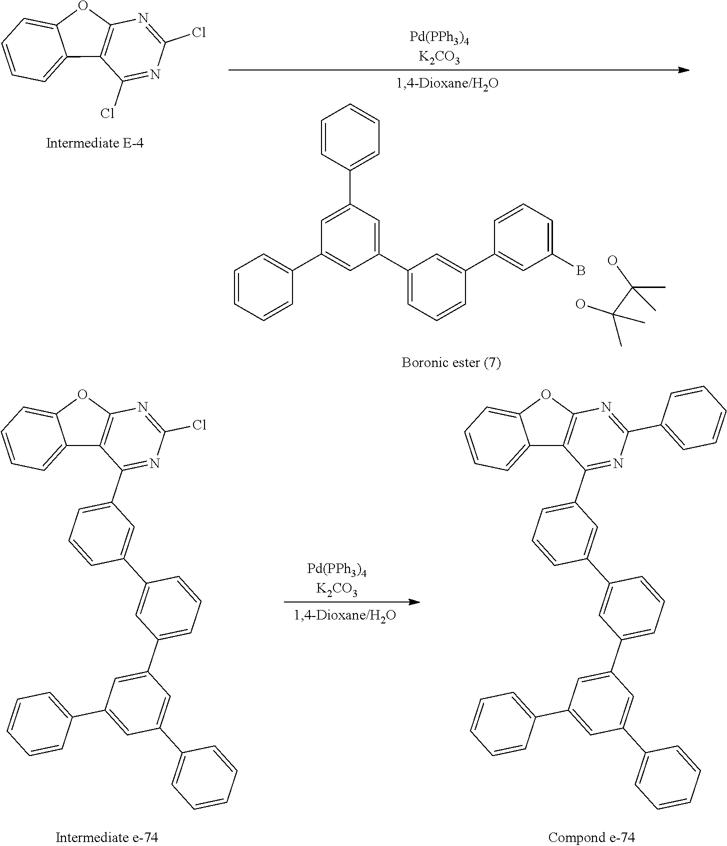

### Synthesis of Intermediate e-74

An intermediate e-74 (10.5 g, Yield: 78%) was synthesized according to the same method as the Synthesis Example ad-25 of the intermediate E-5 except for using boronic ester (7) instead of the phenylboronic acid.

calcd. C40H25ClN2O: C, 82.11; H, 4.31; Cl, 6.06; N, 4.79; O, 2.73. found: C, 82.10; H, 4.28; Cl, 6.05; N, 4.75; O, 2.70.

### Synthesis of Compound 74

A compound e-74 (5.3 g, Yield: 65%) was synthesized according to the same method as the Synthesis Example ad-25 of the compound e-70 except for using the intermediate e-74 instead of the intermediate E-5.

calcd. C46H30N2O: C, 88.15; H, 4.82; N, 4.47; O, 2.55. found: C, 88.15; H, 4.82; N, 4.47; O, 2.55.

### Synthesis Example ad-29

### Synthesis of Compound e-75

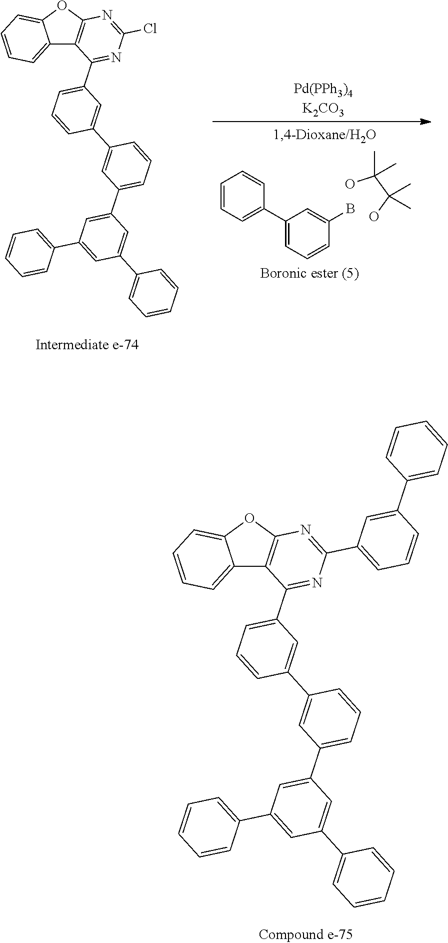

### Synthesis of Compound e-75

A compound e-75 (7.0 g, Yield: 69%) was synthesized according to the same method as the Synthesis Example ad-28 of the compound e-74 except for using boronic ester (5) instead of the phenylboronic acid.

calcd. C52H34N2O: C, 88.86; H, 4.88; N, 3.99; O, 2.28. found: C, 88.85; H, 4.84; N, 3.97; O, 2.28.

### Synthesis Example ad-30

### Synthesis of Compound e-82

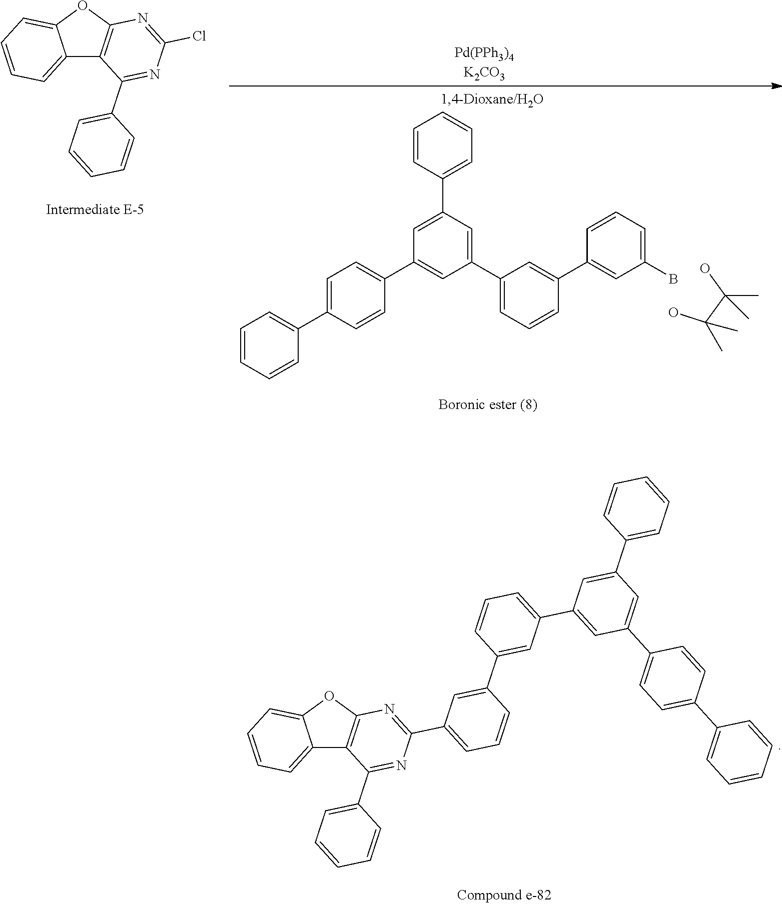

### Synthesis of Compound e-82

A compound e-82 (8.4 g, Yield: 70%) was synthesized according to the same method as the Synthesis Example ad-25 of the compound e-70 except for using boronic ester (8) instead of the boronic ester (7).

calcd. C52H34N2O: C, 88.86; H, 4.88; N, 3.99; O, 2.28. found: C, 88.80; H, 4.81; N, 3.91; O, 2.27.

### Synthesis Example ad-31

### Synthesis of Compound e-84

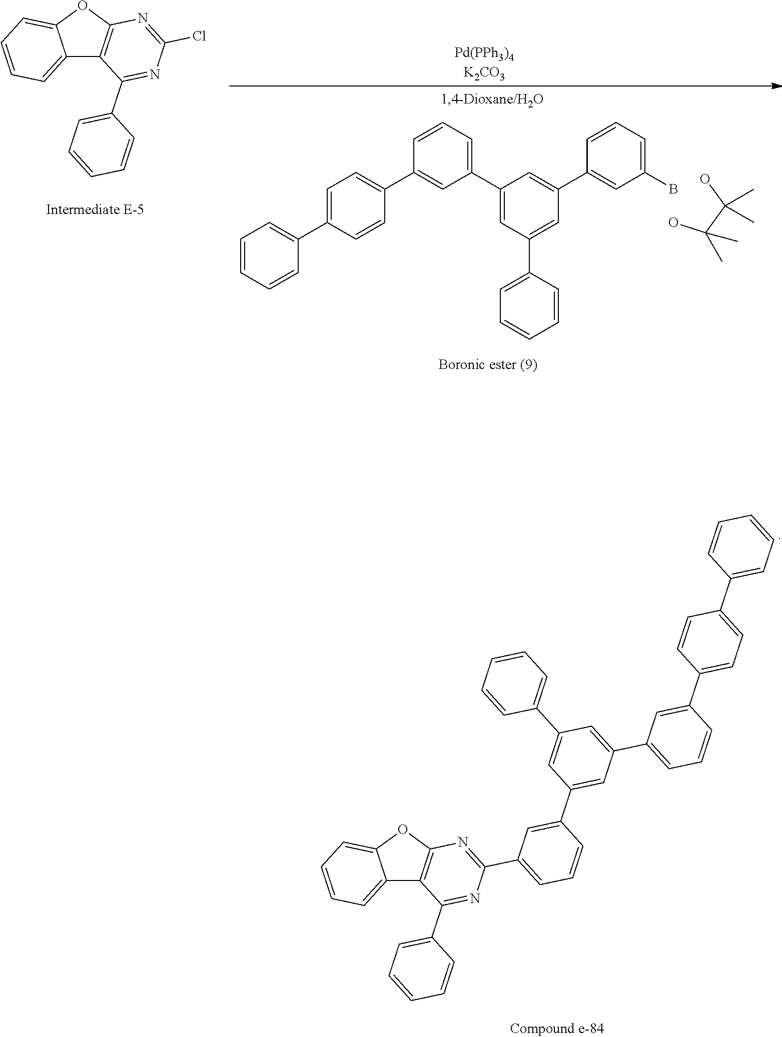

### Synthesis of Compound e-84

A compound e-84 (11.2 g, Yield: 71%) was synthesized according to the same method as the Synthesis Example ad-25 of the compound e-70 except for using boronic ester (9) instead of the boronic ester (7).

calcd. C52H34N2O: C, 88.86; H, 4.88; N, 3.99; O, 2.28. found: C, 88.86; H, 4.85; N, 3.93; O, 2.21.

### Synthesis Example ad-32

### Synthesis of Compound e-88

### Synthesis of Compound e-88

A compound e-88 (6.2 g, Yield: 67%) was synthesized according to the same method as the Synthesis Example ad-25 of the compound e-70 except for using boronic ester (14) instead of the boronic ester (7).

calcd. C52H34N2O: C, 88.86; H, 4.88; N, 3.99; O, 2.28. found: C, 88.83; H, 4.88; N, 3.98; O, 2.26.

### Synthesis Example ad-33

### Synthesis of Compound e-114

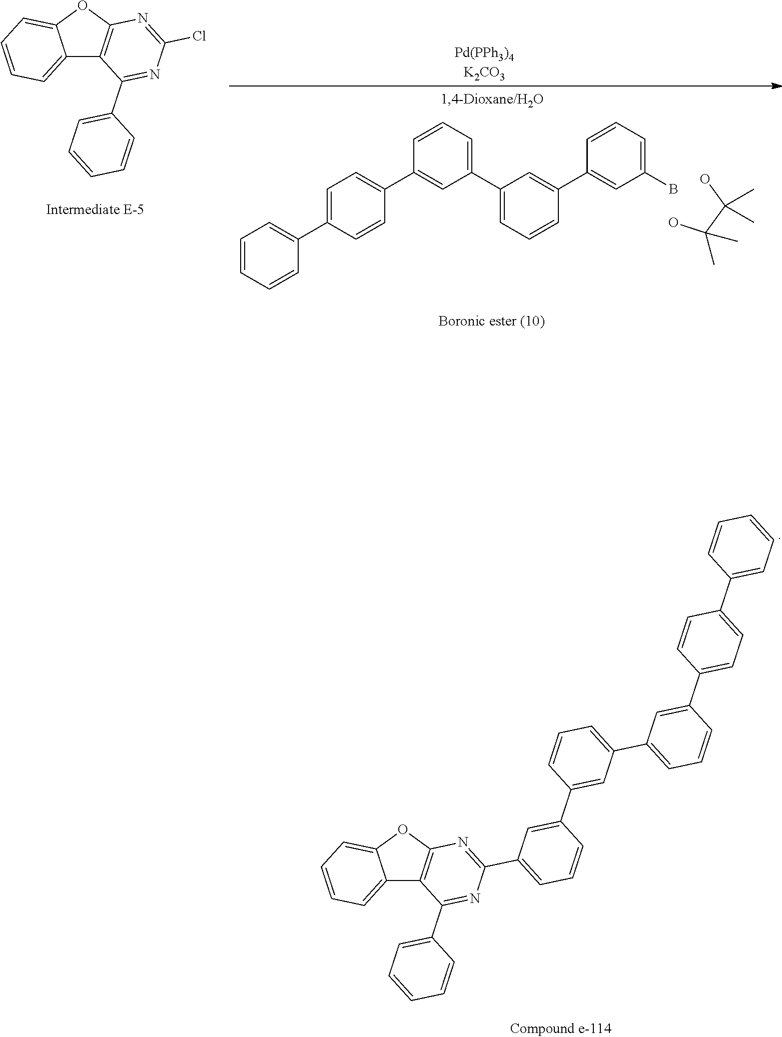

### Synthesis of Compound e-114

A compound e-114 (9.8 g, Yield: 69%) was synthesized according to the same method as the Synthesis Example ad-25 of the compound e-70 except for using boronic ester (10) instead of the boronic ester (7).

calcd. C46H30N2O: C, 88.15; H, 4.82; N, 4.47; O, 2.55. found: C, 88.13; H, 4.81; N, 4.40; O, 2.51.

### Synthesis Example ad-35

### Synthesis of Compound f-71

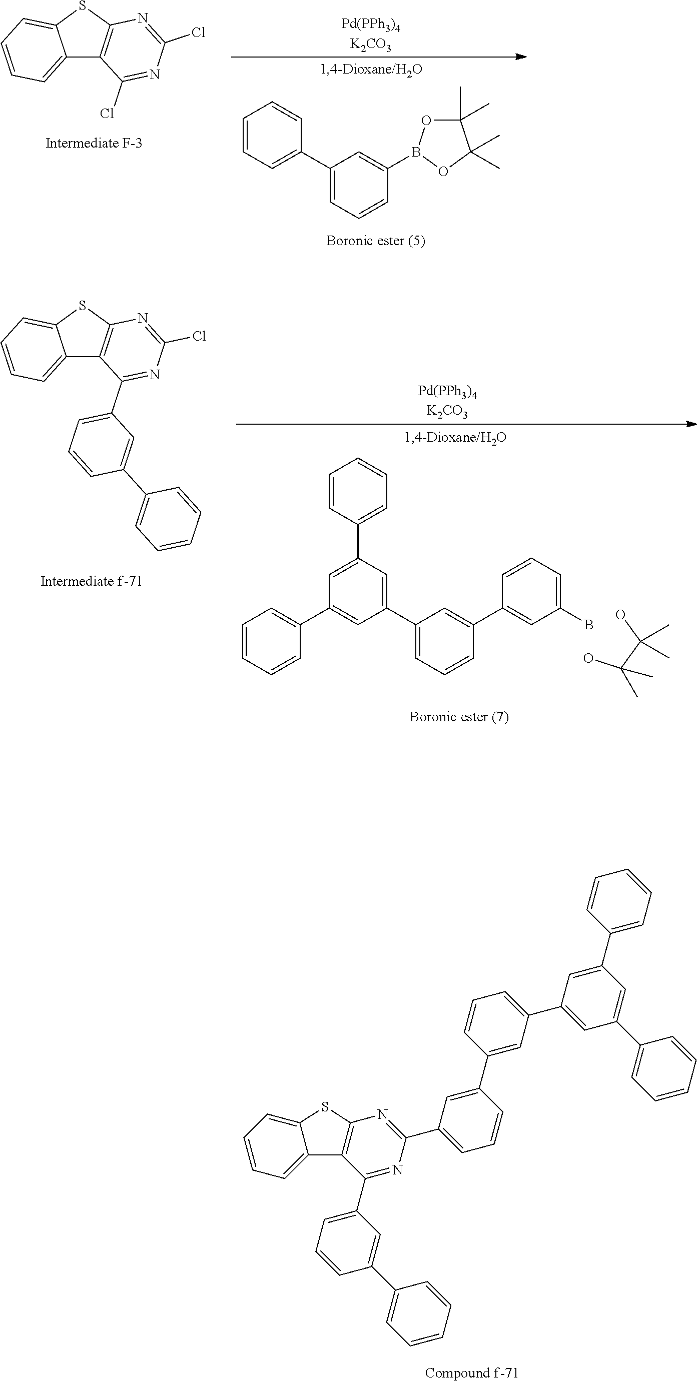

### Synthesis of Intermediate f-71

An intermediate f-71 (11.3 g, Yield: 74%) was synthesized according to the same method as the Synthesis Example ad-26 of the intermediate F-4 except for using boronic ester (5) instead of the phenylboronic acid.

calcd. C22H13ClN2S: C, 70.87; H, 3.51; Cl, 9.51; N, 7.51; S, 8.60. found: C, 70.83; H, 3.50; Cl, 9.89; N, 7.47; S, 8.59.

### Synthesis of Compound f-71

A compound f-71 (9.4 g, Yield: 72%) was synthesized according to the same method as the Synthesis Example ad-26 of the compound f-70 except for using the intermediate f-71 instead of the intermediate F-4.

calcd. C52H34N2S: C, 86.88; H, 4.77; N, 3.90; S, 4.46. found: C, 86.84; H, 4.74; N, 3.88; S, 4.43.

### Synthesis Example ad-36

### Synthesis of Compound f-74

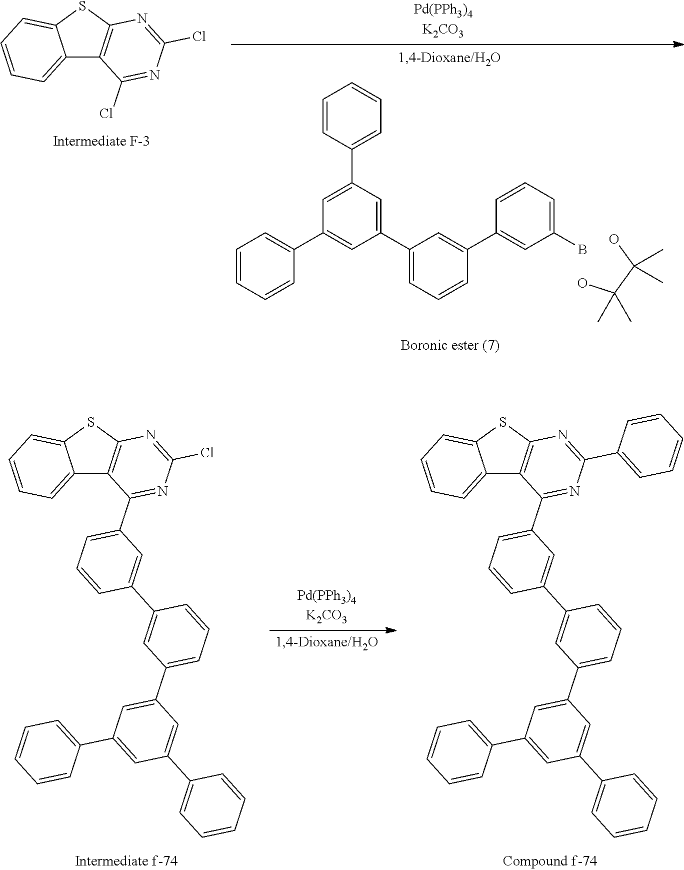

### Synthesis of Intermediate f-74

An intermediate f-74 (8.9 g, Yield: 74%) was synthesized according to the same method as the Synthesis Example ad-26 of the intermediate F-4 except for using boronic ester (7) instead of the phenylboronic acid.

calcd. C40H25ClN2S: C, 79.92; H, 4.19; Cl, 5.90; N, 4.66; S, 5.33. found: C, 79.89; H, 4.18; Cl, 5.87; N, 4.65; S, 5.30.

### Synthesis of Compound f-74

A compound f-74 (7.6 g, Yield: 68%) was synthesized according to the same method as the Synthesis Example ad-26 of the compound f-70 except for using the intermediate f-74 instead of the intermediate F-4.

calcd. C46H30N2S: C, 85.95; H, 4.70; N, 4.36; S, 4.99. found: C, 85.92; H, 4.68; N, 4.35; S, 4.95.

### Synthesis Example ad-37

### Synthesis of Compound f-75

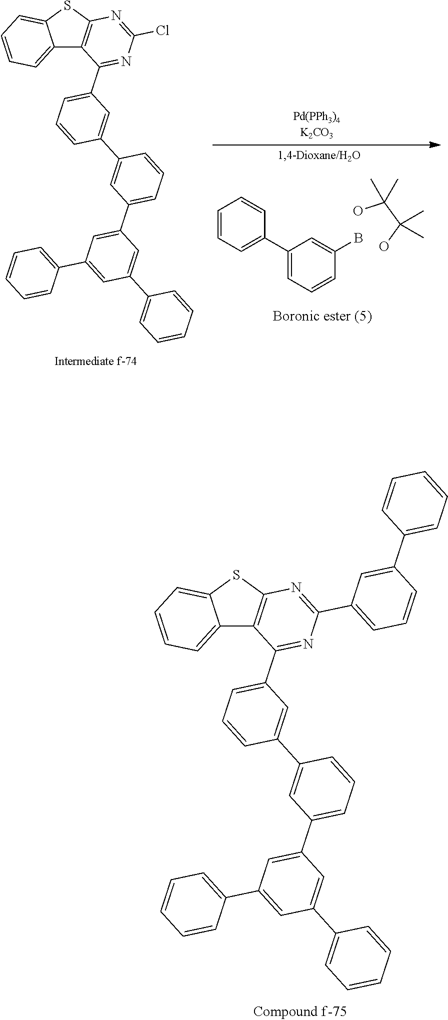

A compound f-75 (6.3 g, Yield: 66%) was synthesized according to the same method as the Synthesis Example ad-36 of the compound f-74 except for using boronic ester (5) instead of the phenylboronic acid.

calcd. C52H34N2S: C, 86.88; H, 4.77; N, 3.90; S, 4.46. found: C, 86.87; H, 4.75; N, 3.89; S, 4.40.

### Synthesis Example ad-38

### Synthesis of Compound f-82

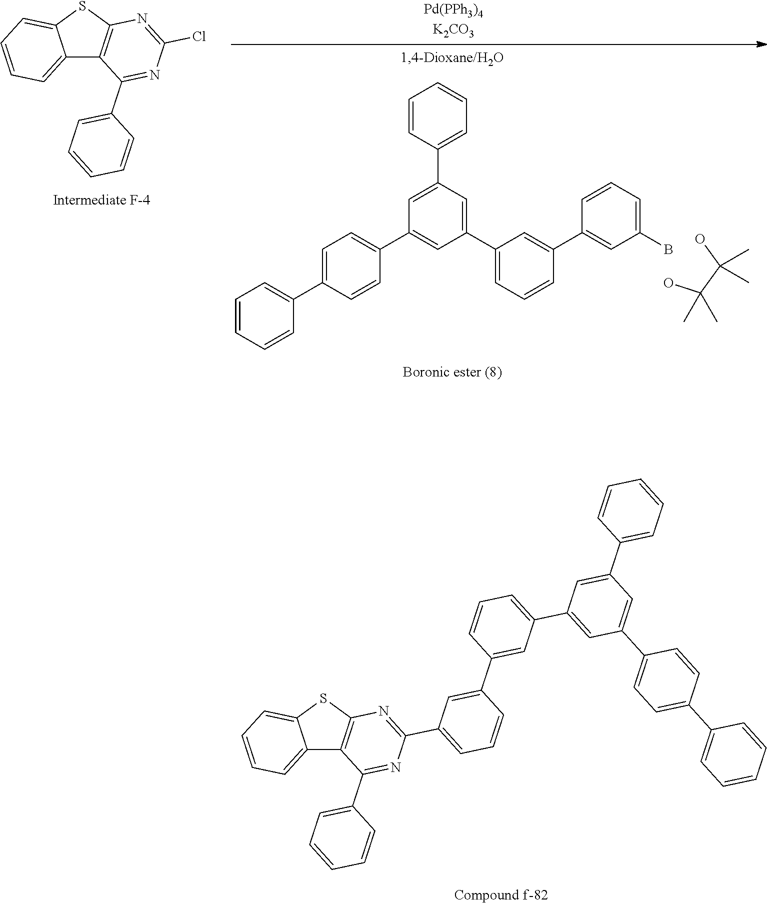

### Synthesis of Compound f-82

A compound f-82 (6.3 g, Yield: 72%) was synthesized according to the same method as the Synthesis Example ad-26 of the compound f-70 except for using boronic ester (8) instead of the boronic ester (7).

calcd. C52H34N2S: C, 86.88; H, 4.77; N, 3.90; S, 4.46. found: C, 86.86; H, 4.75; N, 3.88; S, 4.45.

### Synthesis Example ad-39

### Synthesis of Compound f-84

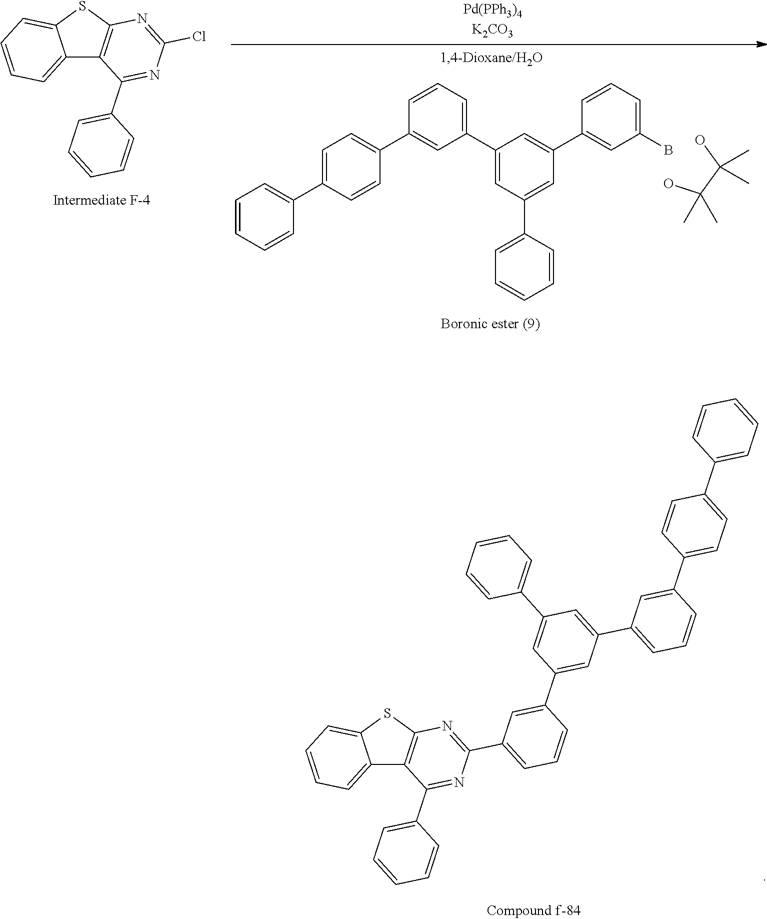

### Synthesis of Compound f-84

A compound f-84 (9.3 g, Yield: 69%) was synthesized according to the same method as the Synthesis Example ad-26 of the compound f-70 except for using boronic ester (9) instead of the boronic ester (7).

calcd. C52H34N2S: C, 86.88; H, 4.77; N, 3.90; S, 4.46. found: C, 86.86; H, 4.76; N, 3.85; S, 4.42.

### Synthesis Example ad-40

### Synthesis of Compound f-88

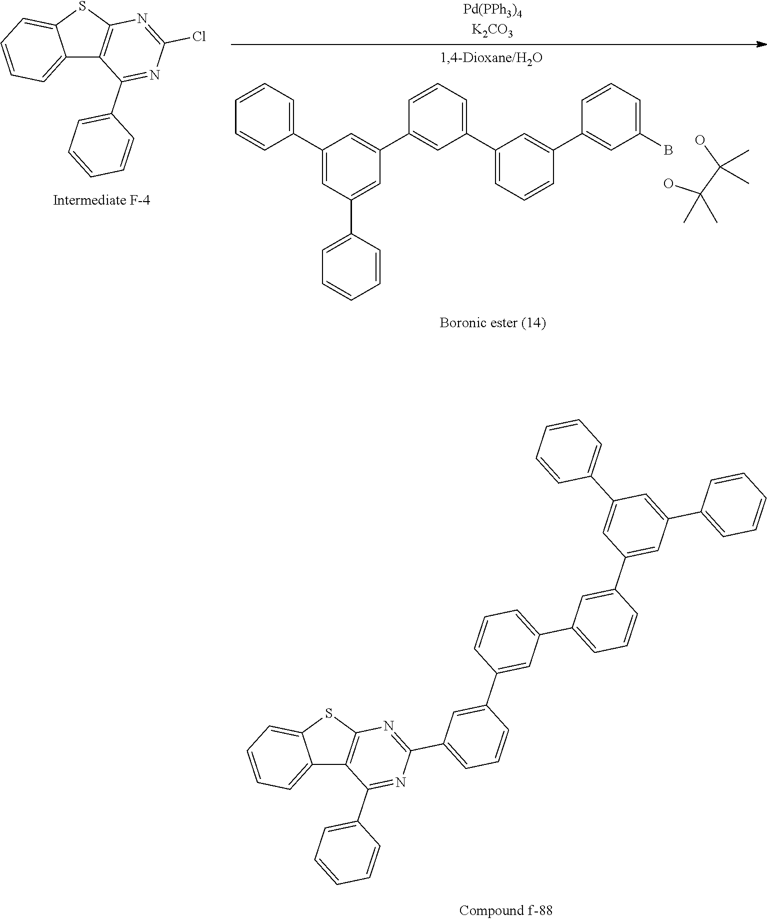

### Synthesis of Compound f-88

A compound f-88 (7.6 g, Yield: 73%) was synthesized according to the same method as the Synthesis Example ad-26 of the compound f-70 except for using boronic ester (14) instead of the boronic ester (7).

calcd. C52H34N2S: C, 86.88; H, 4.77; N, 3.90; S, 4.46. found: C, 86.86; H, 4.73; N, 3.89; S, 4.44.

### Synthesis Example ad-41

### Synthesis of Compound f-114

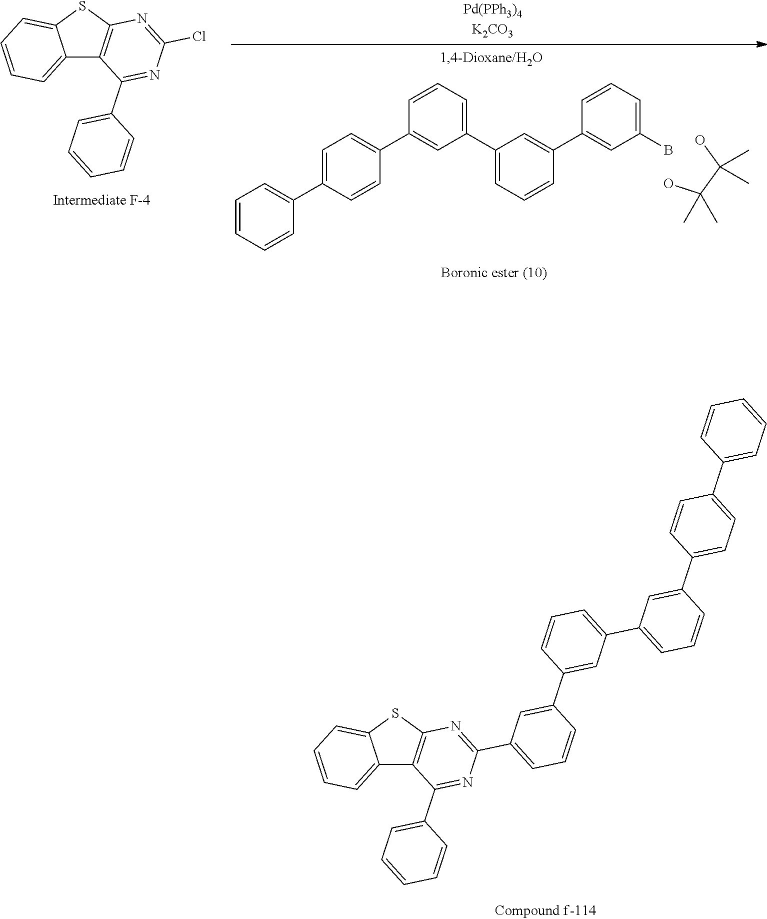

### Synthesis of Compound f-114

A compound f-114 (7.6 g, Yield: 67%) was synthesized according to the same method as the Synthesis Example ad-26 of the compound f-70 except for using boronic ester (10) instead of the boronic ester (7).

calcd. C46H30N2S: C, 85.95; H, 4.70; N, 4.36; S, 4.99. found: C, 85.90; H, 4.69; N, 4.33; S, 4.96.

### Synthesis of Second Host Compound

### Synthesis Example 4

### Synthesis of Compound A1

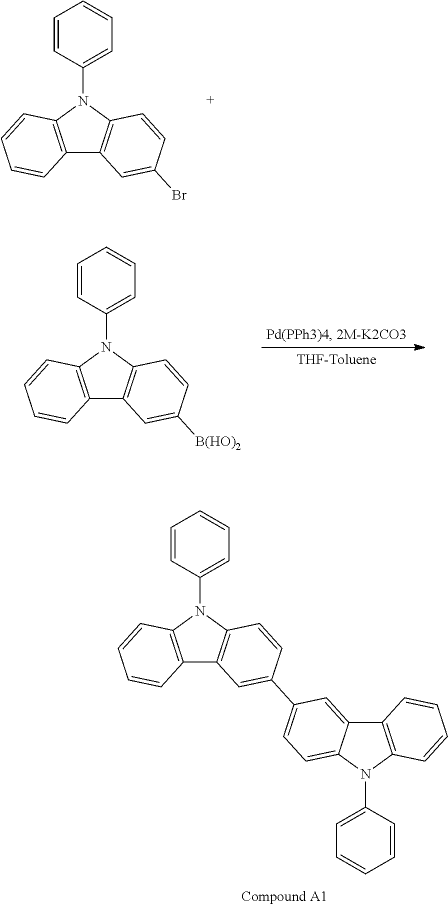

16.62 g (51.59 mmol) of 3-bromo-N-phenylcarbazole, 17.77 g (61.91 mmol) of N-phenylcarbazole-3-ylboronic acid, and 200 mL of a mixture of tetrahydrofuran (THF) and toluene (1:1), and 100 mL of an aqueous solution of 2M potassium carbonate were mixed in a 500-mL round-bottom flask equipped with a stirrer in a nitrogen atmosphere, and 2.98 g (2.58 mmol) of tetrakis(triphenylphosphine)palladium(0) was added thereto, and heated under reflux in a nitrogen atmosphere for about 12 hours. After completion of the reaction, the reaction product was added to methanol to obtain a solid by filtering. This solid was sufficiently washed with water and methanol, and then dried. The resulting product was dissolved in 1 L of chlorobenzene by heating, followed by filtration using silica gel and removing the solvent. The resulting product was dissolved in 500 mL of toluene by heating, followed by recrystallization to obtain Compound A1 (16.05 g, Yield: 64%).

calcd. C₃₆H₂₄N₂: C, 89.23; H, 4.99; N, 5.78. found: C, 89.45; H, 4.89; N, 5.65.

### Synthesis Example 5

### Synthesis of Compound A2

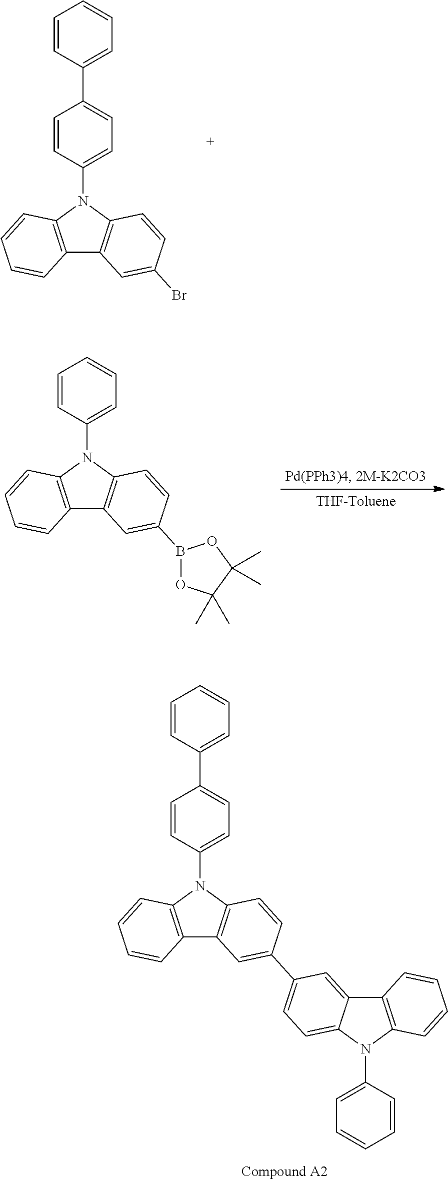 [SMILES: Brc1ccc2c(c1)c1ccccc1n2-c1ccc(-c2ccccc2)cc1 ; CC1(C)OB(c2ccc3c(c2)c2ccccc2n3-c2ccccc2)OC1(C)C ; c1ccc(-c2ccc(-n3c4ccccc4c4cc(-c5ccc6c(c5)c5ccccc5n6-c5ccccc5)ccc43)cc2)cc1]

20.00 g (50.21 mmol) of 3-bromo-N-biphenylcarbazole, 18.54 g (50.21 mmol) of N-phenylcarbazole-3-boronic ester, and 175 mL of a mixture of tetrahydrofuran (THF) and toluene (1:1), and 75 mL of an aqueous solution of 2M potassium carbonate were mixed in a 500-mL round-bottom flask equipped with a stirrer in a nitrogen atmosphere, and 2.90 g (2.51 mmol) of tetrakis(triphenylphosphine)palladium(0) was added thereto, and heated under reflux in a nitrogen atmosphere for about 12 hours. After completion of the reaction, the reaction product was added to methanol to obtain a solid by filtering. This solid was sufficiently washed with water and methanol, and then dried. The resulting product was dissolved in 700 mL of chlorobenzene by heating, followed by filtration using silica gel and removing the solvent. The resulting product was dissolved in 400 mL of chlorobenzene by heating, followed by recrystallization to obtain Compound A2 (19.15 g, Yield: 68%).

calcd. C₄₂H₂₈N₂: C, 89.97; H, 5.03; N, 5.00. found: C, 89.53; H, 4.92; N, 4.89.

### Synthesis Example 6

### Synthesis of Compound A5

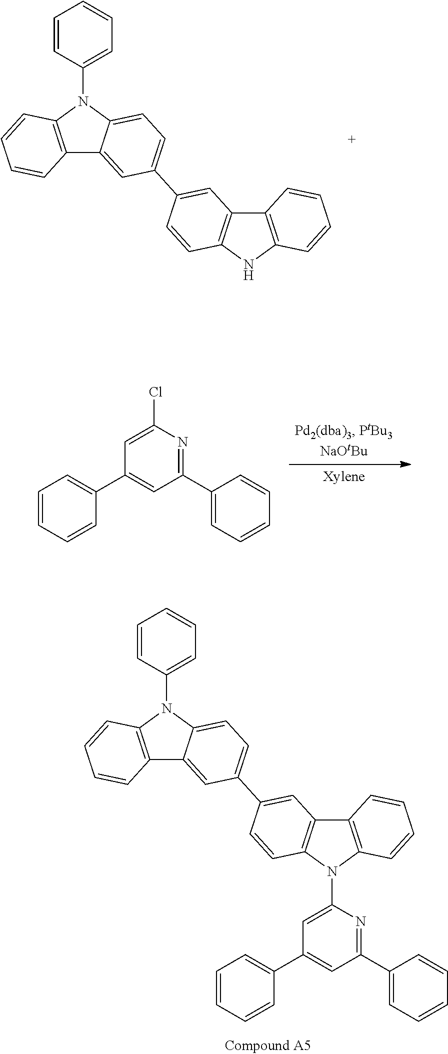 [SMILES: c1ccc(-n2c3ccccc3c3cc(-c4ccc5[nH]c6ccccc6c5c4)ccc32)cc1 ; Clc1cc(-c2ccccc2)cc(-c2ccccc2)n1 ; c1ccc(-c2cc(-c3ccccc3)nc(-n3c4ccccc4c4cc(-c5ccc6c(c5)c5ccccc5n6-c5ccccc5)ccc43)c2)cc1]

12.81 g (31.36 mmol) of N-phenyl-3,3-bicarbazole, 8.33 g (31.36 mmol) of 2-chloro-di-4,6-phenylpyridine, 6.03 g (62.72 mmol) of sodium t-butoxide, 1.80 g (3.14 mmol) of tris(dibenzylideneacetone)dipalladium, and 2.6 mL of tri-t-butylphosphine (50% in toluene) were added to 200 mL of xylene in a 500-mL round-bottom flask, and heated under reflux in a nitrogen atmosphere for about 15 hours. The resulting mixture was added to 600 mL of methanol to obtain crystalline solid powder by filtering. The resulting product was dissolved in dichlorobenzene and filtered using Silica gel/Celite, followed by removing an appropriate amount of the organic solvent and recrystallization with methanol to obtain Compound A5 (13.5 g, Yield: 68%).

calcd. C₄₇H₃₁N₃: C, 88.51; H, 4.90; N, 6.59. found: C, 88.39; H, 4.64; N, 6.43.

### Synthesis Example 7

### Synthesis of Compound A15

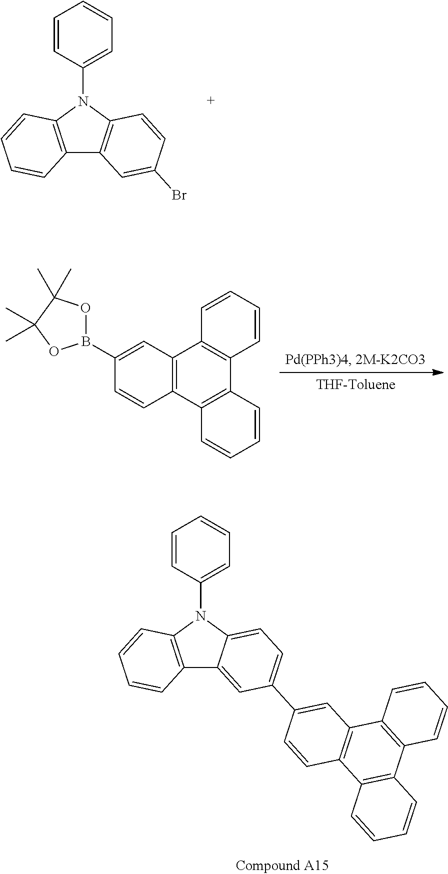 [SMILES: Brc1ccc2c(c1)c1ccccc1n2-c1ccccc1 ; CC1(C)OB(c2ccc3c4ccccc4c4ccccc4c3c2)OC1(C)C ; c1ccc(-n2c3ccccc3c3cc(-c4ccc5c6ccccc6c6ccccc6c5c4)ccc32)cc1]

10.00 g (31.04 mmol) of 3-bromo-N-phenylcarbazole, 10.99 g (31.04 mmol) of 2-triphenylene boronic ester, 150 mL of a mixture of tetrahydrofuran (THF) and toluene (1:1), and 75 mL of an aqueous solution of 2M potassium carbonate were mixed in a 500-mL round-bottom flask equipped with a stirrer in a nitrogen atmosphere, and 1.79 g (1.55 mmol) of tetrakis(triphenylphosphine)palladium(0) was added thereto, and heated under reflux in a nitrogen atmosphere for about 12 hours. After completion of the reaction, the reaction product was added to methanol to obtain a solid by filtering. This solid was sufficiently washed with water and methanol, and then dried. The resulting product was dissolved in 400 mL of chlorobenzene by heating, followed by filtration using silica gel and removing the solvent. The resulting product was dissolved in 300 mL of toluene by heating, followed by recrystallization to obtain Compound A15 (8.74 g, Yield: 60%).

calcd. C₃₆H₂₃N: C, 92.08; H, 4.94; N, 2.98. found: C, 92.43; H, 4.63; N, 2.84.

### Synthesis Example 8

### Synthesis of Compound A17

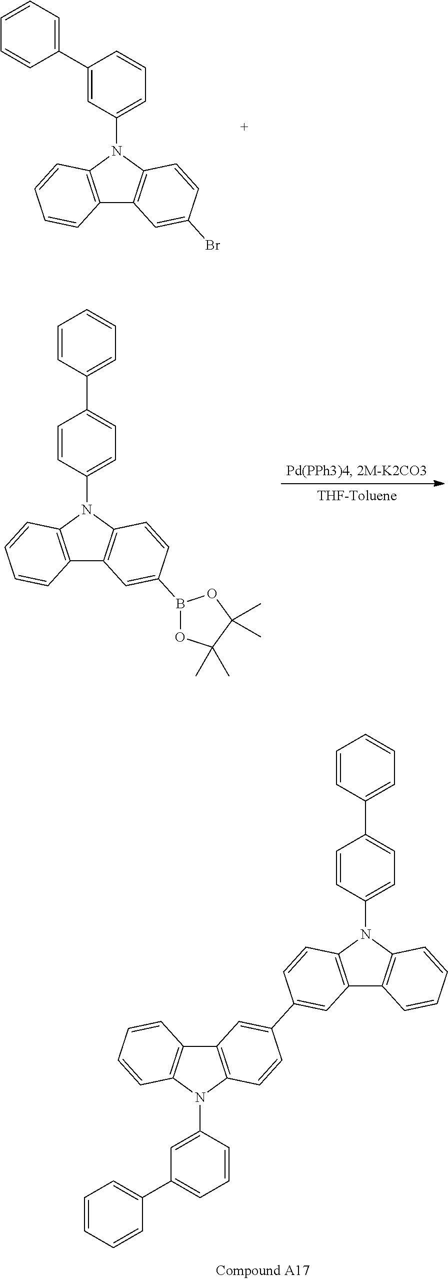 [SMILES: Brc1ccc2c(c1)c1ccccc1n2-c1cccc(-c2ccccc2)c1 ; CC1(C)OB(c2ccc3c(c2)c2ccccc2n3-c2ccc(-c3ccccc3)cc2)OC1(C)C ; c1ccc(-c2ccc(-n3c4ccccc4c4cc(-c5ccc6c(c5)c5ccccc5n6-c5cccc(-c6ccccc6)c5)ccc43)cc2)cc1]

15.00 g (37.66 mmol) of 3-bromo-N-methbiphenylcarbazole, 16.77 g (37.66 mmol) of 3-boronic ester-N-biphenyl carbazole, 200 mL of a mixture of tetrahydrofuran (THF) and toluene (1:1), and 100 mL of an aqueous solution of 2M potassium carbonate were mixed in a 500-mL round-bottom flask equipped with a stirrer in a nitrogen atmosphere, and 2.18 g (1.88 mmol) of tetrakis(triphenylphosphine)palladium(0) was added thereto, and heated under reflux in a nitrogen atmosphere for about 12 hours. After completion of the reaction, the reaction product was added to methanol to obtain a solid by filtering. This solid was sufficiently washed with water and methanol, and then dried. The resulting product was dissolved in 500 mL of chlorobenzene by heating, followed by filtration using silica gel and removing the solvent. The resulting product was dissolved in 400 mL of toluene by heating, followed by recrystallization to obtain Compound A1 (16.07 g, Yield: 67%).

calcd. C₄₈H₃₂N₂: C, 90.54; H, 5.07; N, 4.40. found: C, 90.71; H, 5.01; N, 4.27.

### Synthesis Example ad-42

### Synthesis of Compound A63

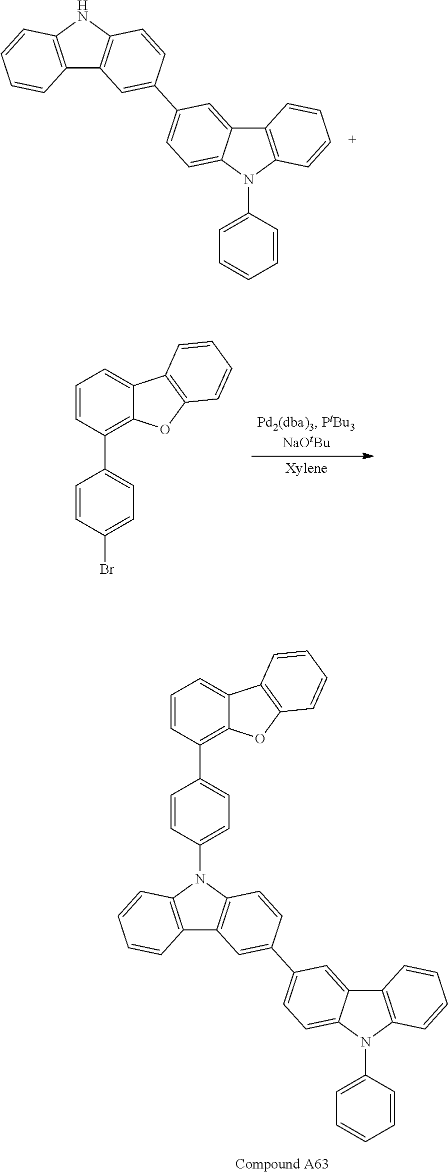 [SMILES: c1ccc(-n2c3ccccc3c3cc(-c4ccc5[nH]c6ccccc6c5c4)ccc32)cc1 ; Brc1ccc(-c2cccc3c2oc2ccccc23)cc1 ; c1ccc(-n2c3ccccc3c3cc(-c4ccc5c(c4)c4ccccc4n5-c4ccc(-c5cccc6c5oc5ccccc56)cc4)ccc32)cc1]

6.3 g (15.4 mmol) of N-phenyl-3,3-bicarbazole, 5.0 g (15.4 mmol) of 4-(4-bromophenyl)dibenzo[b,d]furan, 3.0 g (30.7 mmol) of sodium t-butoxide, 0.9 g (1.5 mmol) of tris(dobenzylideneacetone)dipalladium and 1.2 mL of tri t-butylphosphine (50% in toluene) were mixed with 100 mL of xylene in a 250 mL round flask and then, heated under reflux in a nitrogen atmosphere for 15 hours. The obtained mixture was added to 300 mL of methanol to crystallize a solid, and the solid was filtered, dissolved in dichlorobenzene, and filtered with silica gel/Celite and then, recrystallized with methanol after removing an appropriate amount of an organic solvent therefrom, obtaining the intermediate A63 (7.3 g, Yield: 73%).

calcd. C48H30N2O: C, 88.59; H, 4.65; N, 4.30; O, 2.46. found: C, 88.56; H, 4.62; N, 4.20; O, 2.43.

### Synthesis Example ad-43

### Synthesis of Compound A64

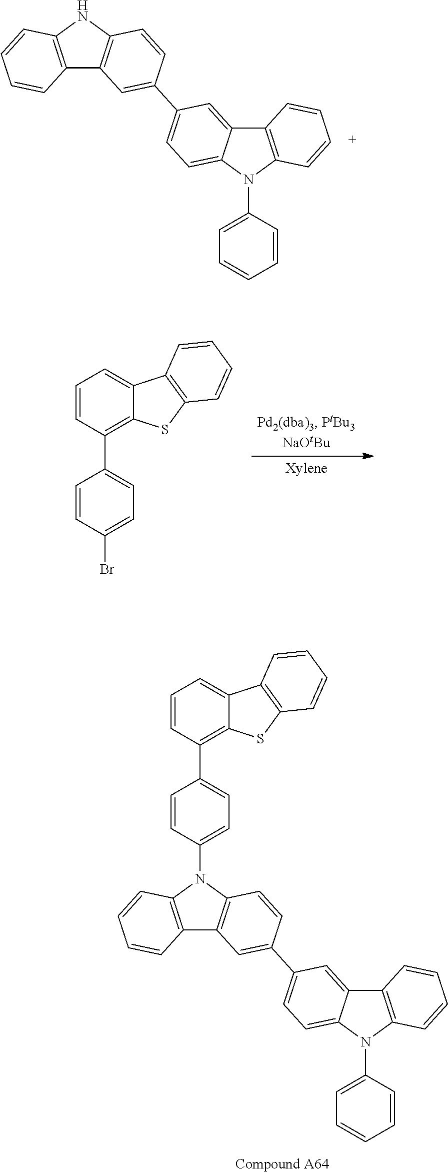 [SMILES: c1ccc(-n2c3ccccc3c3cc(-c4ccc5[nH]c6ccccc6c5c4)ccc32)cc1 ; Brc1ccc(-c2cccc3c2sc2ccccc23)cc1 ; c1ccc(-n2c3ccccc3c3cc(-c4ccc5c(c4)c4ccccc4n5-c4ccc(-c5cccc6c5sc5ccccc56)cc4)ccc32)cc1]

6.1 g (15.0 mmol) of N-phenyl-3,3-bicarbazole, 5.1 g (15.0 mmol) of 4-(4-bromophenyl)dibenzo[b,d]thiophene, 2.9 g (30.0 mmol) of sodium t-butoxide, 0.9 g (1.5 mmol) of tris(dibenzylideneacetone)dipalladium and 1.2 mL of tri t-butylphosphine (50% in toluene) were mixed with 100 mL of xylene in a 250 mL round flask and then, heated under reflux in a nitrogen atmosphere for 15 hours. The obtained mixture was added to 300 mL of methanol to crystallize a solid, and the solid was filtered, dissolved in dichlorobenzene, and filtered with silica gel/Celite filter and then, recrystallized with methanol after removing an appropriate amount of an organic solvent, obtaining the intermediate A64 (6.7 g, Yield: 67%).

calcd. C48H30N2S: C, 86.46; H, 4.53; N, 4.20; S, 4.81. found: C, 86.41; H, 4.51; N, 4.18; S, 4.80.

### Synthesis Example 9

### Synthesis of Compound B2

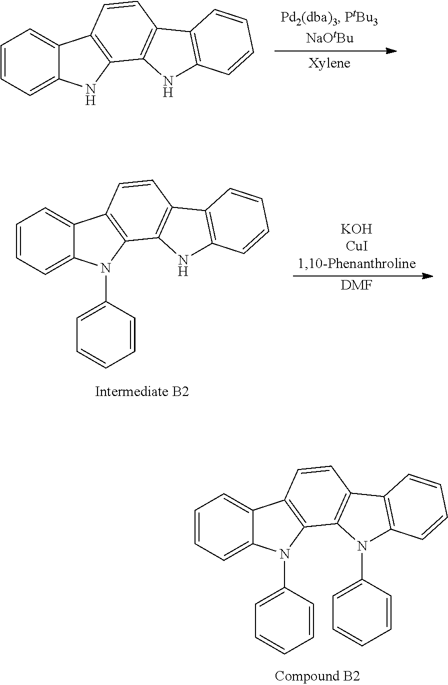

### Synthesis of Intermediate B2

39.99 g (156.01 mmol) of indolocarbazole, 26.94 g (171.61 mmol) of bromobenzene, 22.49 g (234.01 mmol) of sodium t-butoxide, 4.28 g (4.68 mmol) of tris(dibenzylideneacetone)dipalladium, and 2.9 mL of tri-t-butylphosphine (50% in toluene) were added to 500 mL of xylene in a 1000-mL round-bottom flask, and mixed and heated under reflux in a nitrogen atmosphere for about 15 hours. The resulting mixture was added to 1000 mL of methanol to obtain crystalline solid powder by filtering. The resulting product was dissolved in dichlorobenzene and filtered using Silica gel/Celite, followed by removing an appropriate amount of the organic solvent and recrystallization with methanol to obtain Intermediate B2 (23.01 g, Yield: 44%).

calcd. C₂₄H₁₆N₂: C, 86.72; H, 4.85; N, 8.43. found: C, 86.72; H, 4.85; N, 8.43.

### Synthesis of Compound B2

22.93 g (69.03 mmol) of Intermediate B2, 11.38 g (72.49 mmol) of bromobenzene, 4.26 g (75.94 mmol) of potassium hydroxide, 13.14 g (69.03 mmol) of cupper iodide, and 6.22 g (34.52 mmol) of 1,10-phenanthroline were added to 230 mL of dimethylformamide (DMF) in a 500-mL round-bottom flask, and heated under reflux in a nitrogen atmosphere for about 15 hours. The resulting mixture was added to 1000 mL of methanol to obtain crystalline solid powder by filtering. The resulting product was dissolved in dichlorobenzene and filtered using Silica gel/Celite, followed by removing an appropriate amount of the organic solvent and recrystallization with methanol to obtain Compound B2 (12.04 g, Yield: 43%).

calcd. C₃₀H₂₀N₂: C, 88.21; H, 4.93; N, 6.86. found: C, 88.21; H, 4.93; N, 6.86.

### Evaluation Example 1

### Evaluation of HOMO, LUMO, and Triplet (T1) Energy Levels of Synthesized Compounds

The highest occupied molecular orbital (HOMO) energy levels, lowest unoccupied molecular orbital (LUMO) energy levels, and T1 energy levels of the synthesized compounds were evaluated according to the methods described in Table 2 below. The results are shown in Table 1 and 3.

TABLE 2

| | |
|---|---|
| HOMO | Each of the compounds was diluted in CHCl₃ to a |
| energy | concentration of 1 × 10⁻⁵M, and then UV absorption |
| level | spectra thereof were measured at room temperature |
| evaluation | using a spectrometer (Shimadzu UV-350 |
| method | Spectrometer). A HOMO energy level of the |
| | compound was calculated based on the optical |
| | band gap (Eg) of the absorption spectrum edge. |
| LUMO | A potential (V)-current (A) plot of each of the |
| energy | compounds was obtained using cyclic voltammetry |
| level | (CV) (Electrolyte: 0.1M Bu₄NClO₄/Solvent: CH₂Cl₂/ |
| evaluation | Electrode: 3-electrode system (working electrode: GC, |
| method | reference electrode: Ag/AgCl, auxiliary electrode: Pt)), |
| | and a LUMO energy of the compound was calculated |
| | based on the reduction onset potential in the potential- |
| | current plot. |
| T1 energy | A mixture of each of the compounds and toluene |
| level | (prepared by dissolving 1 mg of the compound in 3 cc |
| evaluation | of toluene) was put in a quartz cell, which was then |
| method | placed in liquid nitrogen (77K) for photoluminescence |
| | spectroscopy. Photoluminescence spectra of the |
| | compounds were measured using a photoluminescence |
| | spectrometer, and then compared with those at room |
| | temperature to analyze only peaks appearing at low |
| | temperature. A T1 energy level of each of the compounds |
| | was calculated based on the low-temperature peaks. |

TABLE 3

| | HOMO (eV) | LUMO (eV) | T1 energy |
|---|---|---|---|
| Compound No. | (found) | (found) | level (eV) |
| 30 | −5.531 | −1.739 | 2.713 |
| 29 | −5.402 | −1.746 | 2.734 |
| 27 | −5.548 | −1.753 | 2.698 |

Referring to Table 1 and 3, the synthesized compounds were found to have electrical characteristics suitable for use as materials for organic light-emitting devices.

### Evaluation Example 2

### Thermal Characteristics Evaluation of Compounds

Thermal analysis of each of the synthesized compounds was performed using thermo gravimetric analysis (TGA) and differential scanning calorimetry (DSC) (N₂ atmosphere, temperature range: room temperature to 800° C. (10° C./min)-TGA, room temperature to 400° C.-DSC, Pan Type: Pt Pan in disposable Al Pan (TGA), disposable Al pan (DSC)). The results are shown in Table 4. Referring to Table 4, the synthesized compounds were found to have good thermal stabilities.

TABLE 4

| Compound No. | Tg | Tc | Tm |
|---|---|---|---|
| 30 | 128 | 246 | 261 |
| 29 | 116 | 185 | 250 |
| 27 | 129 | 223 | 267 |

### Example ad-1

An glass substrate with an ITO electrode was cut to a size of 50 mm×50 mm×0.5 mm, washed by sonication in acetone isopropyl alcohol and then in pure water each for 15 minutes, and washed with UV ozone for 30 minutes.

m-MTDATA was vacuum-deposited on the ITO electrode on the glass substrate at a deposition rate of 1 Å/sec to form an HIL having a thickness of 600 Å, and then α-NPB was vacuum-deposited on the HIL at a deposition rate of 1 Å/sec to form a HTL having a thickness of 300 Å. Subsequently, Ir(ppy)₃ (dopant) and Compound b-41 (host) were co-deposited on the HTL at a deposition rate of about 0.1 Å/sec and about 1 Å/sec, respectively, to form an EML having a thickness of about 400 Å. BAlq was vacuum-deposited on the EML at a deposition rate of about 1 Å/sec to form an hole blocking layer (HBL) having a thickness of 50 Å, and then Alq₃ was vacuum-deposited on the HBL to form a HTL having a thickness of 300 Å. LiF and A1 were sequentially vacuum-deposited on the ETL to form an EIL having a thickness of about 10 Å and a cathode having a thickness of 2000 Å, respectively, thereby manufacturing an organic light-emitting device.

### Example ad-2

An organic light-emitting device was manufactured in the same manner as in Example ad-1, except that Compound b-71, instead of Compound b-41, was used as a host to form the EML.

### Example 1

An organic light-emitting device was manufactured in the same manner as in Example ad-1, except that Compound 29, instead of Compound b-41, was used as a host to form the EML.

### Example 2

An organic light-emitting device was manufactured in the same manner as in Example ad-1, except that Compound 30, instead of Compound b-41, was used as a host to form the EML.

### Example ad-3

An organic light-emitting device was manufactured in the same manner as in Example ad-1, except that Compound 27, instead of Compound b-41, was used as a host to form the EML.

### Example ad-4

An organic light-emitting device was manufactured in the same manner as in Example ad-1, except that Compound a-30, instead of Compound b-41, was used as a host to form the EML.

### Example ad-5

An organic light-emitting device was manufactured in the same manner as in Example ad-1, except that Compound a-40, instead of Compound b-41, was used as a host to form the EML.

### Example ad-6

An organic light-emitting device was manufactured in the same manner as in Example ad-1, except that Compound a-41, instead of Compound b-41, was used as a host to form the EML.

### Example ad-7

An organic light-emitting device was manufactured in the same manner as in Example ad-1, except that Compound a-42, instead of Compound b-41, was used as a host to form the EML.

### Example ad-8

An organic light-emitting device was manufactured in the same manner as in Example ad-1, except that Compound a-46, instead of Compound b-41, was used as a host to form the EML.

### Example ad-9

An organic light-emitting device was manufactured in the same manner as in Example ad-1, except that Compound a-56, instead of Compound b-41, was used as a host to form the EML.

### Example ad-10

An organic light-emitting device was manufactured in the same manner as in Example ad-1, except that Compound a-70, instead of Compound b-41, was used as a host to form the EML.

### Example ad-11

An organic light-emitting device was manufactured in the same manner as in Example ad-1, except that Compound a-71, instead of Compound b-41, was used as a host to form the EML.

### Example ad-12

An organic light-emitting device was manufactured in the same manner as in Example ad-1, except that Compound a-74, instead of Compound b-41, was used as a host to form the EML.

### Example ad-13

An organic light-emitting device was manufactured in the same manner as in Example ad-1, except that Compound a-75, instead of Compound b-41, was used as a host to form the EML.

### Example ad-14

An organic light-emitting device was manufactured in the same manner as in Example ad-1, except that Compound a-82, instead of Compound b-41, was used as a host to form the EML.

### Example ad-15

An organic light-emitting device was manufactured in the same manner as in Example ad-1, except that Compound a-84, instead of Compound b-41, was used as a host to form the EML.

### Example ad-16

An organic light-emitting device was manufactured in the same manner as in Example ad-1, except that Compound a-114, instead of Compound b-41, was used as a host to form the EML.

### Example ad-17

An organic light-emitting device was manufactured in the same manner as in Example ad-1, except that Compound a-110, instead of Compound b-41, was used as a host to form the EML.

### Example ad-18

An organic light-emitting device was manufactured in the same manner as in Example ad-1, except that Compound a-112, instead of Compound b-41, was used as a host to form the EML.

### Example ad-19

An organic light-emitting device was manufactured in the same manner as in Example ad-1, except that Compound c-40, instead of Compound b-41, was used as a host to form the EML.

### Example ad-20

An organic light-emitting device was manufactured in the same manner as in Example ad-1, except that Compound c-50, instead of Compound b-41, was used as a host to form the EML.

### Example ad-21

An organic light-emitting device was manufactured in the same manner as in Example ad-1, except that Compound d-119, instead of Compound b-41, was used as a host to form the EML.

### Example ad-22

An organic light-emitting device was manufactured in the same manner as in Example ad-1, except that Compound e-70, instead of Compound b-41, was used as a host to form the EML.

### Example ad-23

An organic light-emitting device was manufactured in the same manner as in Example ad-1, except that Compound f-70, instead of Compound b-41, was used as a host to form the EML.

### Example ad-24

An organic light-emitting device was manufactured in the same manner as in Example ad-1, except that Compound e-71, instead of Compound b-41, was used as a host to form the EML.

### Example ad-25

An organic light-emitting device was manufactured in the same manner as in Example ad-1, except that Compound e-74, instead of Compound b-41, was used as a host to form the EML.

### Example ad-26

An organic light-emitting device was manufactured in the same manner as in Example ad-1, except that Compound e-75, instead of Compound b-41, was used as a host to form the EML.

### Example ad-27

An organic light-emitting device was manufactured in the same manner as in Example ad-1, except that Compound e-82, instead of Compound b-41, was used as a host to form the EML.

### Example ad-28

An organic light-emitting device was manufactured in the same manner as in Example ad-1, except that Compound e-84, instead of Compound b-41, was used as a host to form the EML.

### Example ad-29

An organic light-emitting device was manufactured in the same manner as in Example ad-1, except that Compound e-88, instead of Compound b-41, was used as a host to form the EML.

### Example ad-30

An organic light-emitting device was manufactured in the same manner as in Example ad-1, except that Compound e-114, instead of Compound b-41, was used as a host to form the EML.

### Example ad-31

An organic light-emitting device was manufactured in the same manner as in Example ad-1, except that Compound f-71, instead of Compound b-41, was used as a host to form the EML.

### Example ad-32

An organic light-emitting device was manufactured in the same manner as in Example ad-1, except that Compound f-74, instead of Compound b-41, was used as a host to form the EML.

### Example ad-33

An organic light-emitting device was manufactured in the same manner as in Example ad-1, except that Compound f-75, instead of Compound b-41, was used as a host to form the EML.

### Example ad-34

An organic light-emitting device was manufactured in the same manner as in Example ad-1, except that Compound f-82, instead of Compound b-41, was used as a host to form the EML.

### Example ad-35

An organic light-emitting device was manufactured in the same manner as in Example ad-1, except that Compound f-84, instead of Compound b-41, was used as a host to form the EML.

### Example ad-36

An organic light-emitting device was manufactured in the same manner as in Example ad-1, except that Compound f-88, instead of Compound b-41, was used as a host to form the EML.

### Example ad-37

An organic light-emitting device was manufactured in the same manner as in Example ad-1, except that Compound f-114, instead of Compound b-41, was used as a host to form the EML.

### Fabrication of the Organic Light-Emitting Device (the Emission Layer of the Device-Mixed Host)

### Example ad-38

An organic light-emitting device was manufactured in the same manner as in Example ad-1, except that Ir(ppy)₃ (dopant), Compound a-70 (first host), and Compound A1 (second host) were co-deposited in a weight ratio of about 10:45:45 on the HTL to form the EML having a thickness of about 400 Å.

### Example ad-39

An organic light-emitting device was manufactured in the same manner as in Example ad-38, except that Compound A2, instead of Compound A1, was used to form the EML.

### Example ad-40

An organic light-emitting device was manufactured in the same manner as in Example ad-38, except that Compound A5, instead of Compound A1, was used to form the EML.

### Example ad-41

An organic light-emitting device was manufactured in the same manner as in Example ad-38, except that Compound A15, instead of Compound A1, was used to form the EML.

### Example ad-42

An organic light-emitting device was manufactured in the same manner as in Example ad-38, except that Compound A17, instead of Compound A1, was used to form the EML.

### Example ad-43

An organic light-emitting device was manufactured in the same manner as in Example ad-38, except that Compound A63, instead of Compound A1, was used to form the EML.

### Example ad-44

An organic light-emitting device was manufactured in the same manner as in Example ad-38, except that Compound A64, instead of Compound A1, was used to form the EML.

### Example ad-45

An organic light-emitting device was manufactured in the same manner as in Example ad-38, except that Compound B2, instead of Compound A1, was used to form the EML.

### Example ad-46

An organic light-emitting device was manufactured in the same manner as in Example ad-38, except that Ir(ppy)₃ (dopant), Compound a-40 (first host), and Compound A17 (second host) were co-deposited in a weight ratio of about 10:45:45 on the HTL to form the EML having a thickness of about 400 Å.

### Example ad-47

An organic light-emitting device was manufactured in the same manner as in Example ad-46, except that Compound a-71, instead of Compound a-40, was used to form the EML.

### Example ad-48

An organic light-emitting device was manufactured in the same manner as in Example ad-46, except that Compound a-74, instead of Compound a-40, was used to form the EML.

### Example ad-49

An organic light-emitting device was manufactured in the same manner as in Example ad-46, except that Compound a-75, instead of Compound a-40, was used to form the EML.

### Example ad-50

An organic light-emitting device was manufactured in the same manner as in Example ad-46, except that Compound a-82, instead of Compound a-40, was used to form the EML.

### Example ad-51

An organic light-emitting device was manufactured in the same manner as in Example ad-46, except that Compound a-84, instead of Compound a-40, was used to form the EML.

### Example ad-52

An organic light-emitting device was manufactured in the same manner as in Example ad-38, except that Ir(ppy)₃ (dopant), Compound a-75 (first host), and Compound A63 (second host) were co-deposited in a weight ratio of about 10:45:45 on the HTL to form the EML having a thickness of about 400 Å.

### Example ad-53

An organic light-emitting device was manufactured in the same manner as in Example ad-52, except that Compound A64, instead of Compound A63, was used to form the EML.

### Example ad-54

An organic light-emitting device was manufactured in the same manner as in Example ad-46, except that Compound e-75, instead of Compound a-40, was used to form the EML.

### Example ad-55

An organic light-emitting device was manufactured in the same manner as in Example ad-46, except that Compound e-114, instead of Compound a-40, was used to form the EML.

### Example ad-56

An organic light-emitting device was manufactured in the same manner as in Example ad-46, except that Compound f-75, instead of Compound a-40, was used to form the EML.

### Example ad-57

An organic light-emitting device was manufactured in the same manner as in Example ad-46, except that Compound f-114, instead of Compound a-40, was used to form the EML.

### Example ad-58

An organic light-emitting device was manufactured in the same manner as in Example ad-54, except that Compound A64, instead of Compound A17, was used to form the EML.

### Example ad-59

An organic light-emitting device was manufactured in the same manner as in Example ad-55, except that Compound A64, instead of Compound A17, was used to form the EML.

### Example ad-60

An organic light-emitting device was manufactured in the same manner as in Example ad-56, except that Compound A64, instead of Compound A17, was used to form the EML.

### Example ad-61

An organic light-emitting device was manufactured in the same manner as in Example ad-57, except that Compound A64, instead of Compound A17, was used to form the EML.

### Comparative Example 1

An organic light-emitting device was manufactured in the same manner as in Example ad-1, except that Compound A, instead of Compound b-41, was used as a host to form the EML.

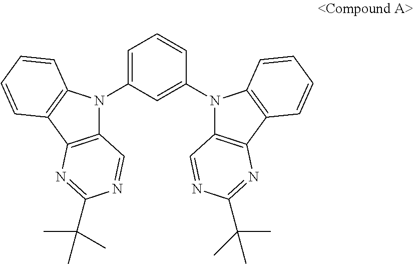 [SMILES: CC(C)(C)c1ncc2c(n1)c1ccccc1n2-c1cccc(-n2c3ccccc3c3nc(C(C)(C)C)ncc32)c1]

### Comparative Example 2

An organic light-emitting device was manufactured in the same manner as in Example ad-1, except that Compound B, instead of Compound b-41, was used as a host to form the EML.

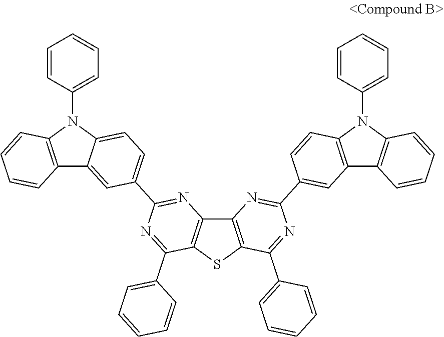 [SMILES: c1ccc(-c2nc(-c3ccc4c(c3)c3ccccc3n4-c3ccccc3)nc3c2sc2c(-c4ccccc4)nc(-c4ccc5c(c4)c4ccccc4n5-c4ccccc4)nc23)cc1]

### Comparative Example 3

An organic light-emitting device was manufactured in the same manner as in Example ad-1, except that Compound C, instead of Compound b-41, was used as a host to form the EML.

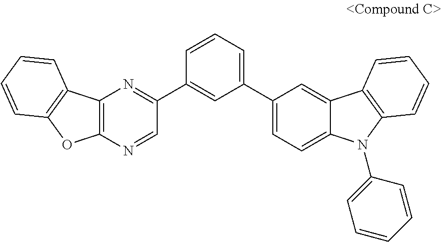 [SMILES: c1ccc(-n2c3ccccc3c3cc(-c4cccc(-c5cnc6oc7ccccc7c6n5)c4)ccc32)cc1]

### Comparative Example 4

An organic light-emitting device was manufactured in the same manner as in Example ad-1, except that Compound D, instead of Compound b-41, was used as a host to form the EML.

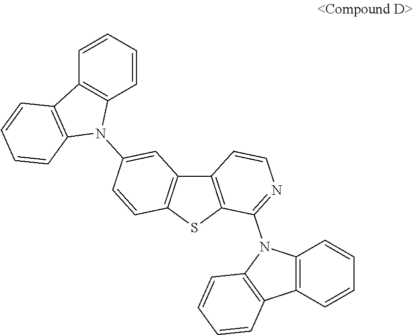 [SMILES: c1ccc2c(c1)c1ccccc1n2-c1ccc2sc3c(-n4c5ccccc5c5ccccc54)nccc3c2c1]

### Example ad-62

### Emission Layer Device (2)-Single Host

An organic light-emitting device was manufactured by using b-116 according to Synthesis Example ad-20 as a host and (piq)₂Ir(acac) as a dopant.

As for an anode, a 1000 Å-thick ITO was used, and as for a cathode, a 1000 Å-thick aluminum (Al) was used. Specifically, a method of manufacturing the organic light-emitting device used a anode obtained by cutting an ITO glass substrate having sheet resistance of 15 Ω/cm² into a size of 50 mm □ 50 mm □ 0.7 mm, ultrasonic wave-cleaning it with acetone, isopropyl alcohol and pure water for 15 minutes respectively and UV ozone-cleaning it for 30 minutes.

On the substrate, a 800 Å-thick hole transport layer (HTL) was formed by depositing N4,N4′-di(naphthalen-1-yl)-N4,N4′-diphenylbiphenyl-4,4′-diamine (NPB) (80 nm) with a vacuum degree of 650×10⁻⁷ Pa at a deposition rate of 0.1 to 0.3 nm/s. Subsequently, a 300 Å-thick emission layer was formed thereon by using b-116 of Synthesis Example ad-20 under the same deposition condition, and herein, (piq)₂Ir(acac) as a phosphorescent dopant was simultaneously deposited therewith.

Herein, 3 wt % of the phosphorescent dopant based on 100 wt % of the emission layer was deposited by adjusting its deposition rate.

Then, a 50 Å-thick hole blocking layer was formed by using bis(2-methyl-8-quinolinolate)-4-(phenylphenolato)aluminum (BAlq) on the emission layer under the same vacuum deposition condition. Subsequently, a 200 Å-thick electron transport layer was formed thereon by depositing Alq3 under the same vacuum deposition condition. On the electron transport layer (ETL), a cathode was formed by sequentially depositing LiF and A1, manufacturing an organic optoelectronic device.

The organic optoelectronic device has a structure of ITO/NPB (80 nm)/EML (b-116 (97 wt %)+(piq)₂1 r(acac) (3 wt %), 30 nm)/Balq (5 nm)/Alq 320 nm/LiF (1 nm)/Al 100 nm.

### Example ad-63

An organic light-emitting device was manufactured according to the same method as Example ad-62 except for using the compound a-108 of Synthesis Example ad-14 instead of the compound b-116 of Synthesis Example ad-20.

### Comparative Example ad-1

An organic light-emitting device was manufactured according to the same method as Example ad-62 except for using CBP having the following structure instead of the compound b-116 of Example ad-62.

NPB, BAlq, CBP and (piq)₂Ir(acac) used to manufacture the organic light-emitting device have a structure as follows.

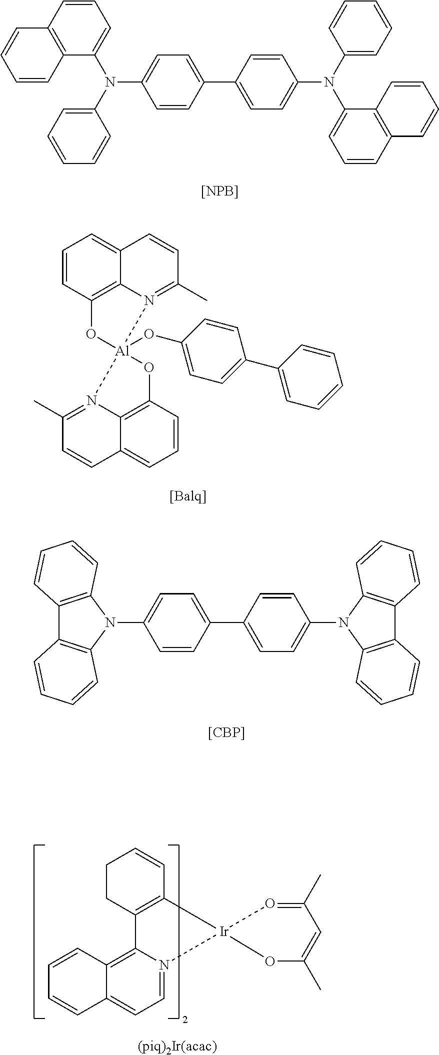

### Evaluation Example 3

### Characteristics Evaluation of Organic Light-Emitting Devices (I)

Driving voltages, current efficiencies, and luminances of the organic light-emitting devices of Examples 1, 2, ad-1 to ad-17, and ad-21 to ad-63 and Comparative Examples 1 to 4 and ad-1 were measured using a PR650 (Spectroscan) Source Measurement Unit (available from Photo Research, Inc.) while supplying power using a Keithley Source-Measure Unit (SMU 236). The specific measurements are described below, and the results are shown in Tables 5 to 7 below.

(1) Measurement of Current Density Change Depending on Voltage Change

Current of each organic light-emitting device was measured by increasing a voltage from 0 V to 10 V by using a current-voltage meter (Keithley 2400), and the measured current value was divided by an area to provide the results.

(2) Measurement of Luminance Change Depending on Voltage Change

Luminance of each organic light-emitting device was measured by increasing a voltage from 0 V to 10 V by using a luminance meter (Minolta Cs-1000A).

(3) Measurement of Luminous Efficiency

The luminance and current density obtained from the above (1) and (2) and a voltage were used to calculate current efficiency (cd/A) at the same current density (10 mA/cm²).

(4) Life-Span

The life-span was obtained by measuring how long the current efficiency (cd/A) decreased by 90% while the luminance (cd/m²) was maintained at 5000 cd/m².

TABLE 5

| | | | Driving | Current | |
|---|---|---|---|---|---|
| | | | voltage | efficiency | Luminance |
| Example | Host | Dopant | (V) | (cd/A) | (cd/m²) |
| Example 1 | Compound | Ir(ppy)₃ | 4.3 | 35 | 6000 |
| | 29 | | | | |
| Example 2 | Compound | Ir(ppy)₃ | 4.5 | 39 | 6000 |
| | 30 | | | | |
| Example ad- | Compound b- | Ir(ppy)3 | 4.6 | 46 | 6000 |
| 1 | 41 | | | | |
| Example ad- | Compound b- | Ir(ppy)3 | 4.5 | 48 | 6000 |
| 2 | 71 | | | | |
| Example ad- | Compound | Ir(ppy)3 | 4.8 | 39 | 6000 |
| 3 | 27 | | | | |
| Example ad- | Compound a- | Ir(ppy)3 | 4.7 | 41 | 6000 |
| 4 | 30 | | | | |
| Example ad- | Compound a- | Ir(ppy)3 | 4.3 | 51 | 6000 |
| 5 | 40 | | | | |
| Example ad- | Compound a- | Ir(ppy)3 | 4.4 | 50 | 6000 |
| 6 | 41 | | | | |
| Example ad- | Compound a- | Ir(ppy)3 | 4.5 | 49 | 6000 |
| 7 | 42 | | | | |
| Example ad- | Compound a- | Ir(ppy)3 | 4.5 | 47 | 6000 |
| 8 | 46 | | | | |
| Example ad- | Compound a- | Ir(ppy)3 | 4.6 | 50 | 6000 |
| 9 | 56 | | | | |
| Example ad- | Compound a- | Ir(ppy)3 | 4.4 | 49 | 6000 |
| 10 | 70 | | | | |
| Example ad- | Compound a- | Ir(ppy)3 | 4.4 | 52 | 6000 |
| 11 | 71 | | | | |
| Example ad- | Compound a- | Ir(ppy)3 | 4.3 | 51 | 6000 |
| 12 | 74 | | | | |
| Example ad- | Compound a- | Ir(ppy)3 | 4.2 | 53 | 6000 |
| 13 | 75 | | | | |
| Example ad- | Compound a- | Ir(ppy)3 | 4.3 | 53 | 6000 |
| 14 | 82 | | | | |
| Example ad- | Compound a- | Ir(ppy)3 | 4.5 | 51 | 6000 |
| 15 | 84 | | | | |
| Example ad- | Compound | Ir(ppy)3 | 4.4 | 50 | 6000 |
| 16 | a-114 | | | | |
| Example ad- | Compound a- | Ir(ppy)3 | 4.5 | 47 | 6000 |
| 17 | 110 | | | | |
| Example ad- | Compound d- | Ir(ppy)3 | 4.6 | 41 | 6000 |
| 21 | 119 | | | | |
| Example ad- | Compound e- | Ir(ppy)3 | 4.5 | 46 | 6000 |
| 22 | 70 | | | | |
| Example ad- | Compound f- | Ir(ppy)3 | 4.4 | 47 | 6000 |
| 23 | 70 | | | | |
| Example ad- | Compound e- | Ir(ppy)3 | 4.4 | 49 | 6000 |
| 24 | 71 | | | | |
| Example ad- | Compound e- | Ir(ppy)3 | 4.5 | 49 | 6000 |
| 25 | 74 | | | | |
| Example ad- | Compound e- | Ir(ppy)3 | 4.4 | 50 | 6000 |
| 26 | 75 | | | | |
| Example ad- | Compound e- | Ir(ppy)3 | 4.4 | 47 | 6000 |
| 27 | 82 | | | | |
| Example ad- | Compound e- | Ir(ppy)3 | 4.5 | 48 | 6000 |
| 28 | 84 | | | | |
| Example ad- | Compound e- | Ir(ppy)3 | 4.3 | 48 | 6000 |
| 29 | 88 | | | | |
| Example ad- | Compound e- | Ir(ppy)3 | 4.3 | 51 | 6000 |
| 30 | 114 | | | | |
| Example ad- | Compound f- | Ir(ppy)3 | 4.3 | 50 | 6000 |
| 31 | 71 | | | | |
| Example ad- | Compound f- | Ir(ppy)3 | 4.4 | 49 | 6000 |
| 32 | 74 | | | | |
| Example ad- | Compound f- | Ir(ppy)3 | 4.5 | 50 | 6000 |
| 33 | 75 | | | | |
| Example ad- | Compound f- | Ir(ppy)3 | 4.6 | 48 | 6000 |
| 34 | 82 | | | | |
| Example ad- | Compound f- | Ir(ppy)3 | 4.4 | 49 | 6000 |
| 35 | 84 | | | | |
| Example ad- | Compound | Ir(ppy)3 | 4.4 | 52 | 6000 |
| 36 | [symbol] f-88 | | | | |
| Example ad- | Compound f- | Ir(ppy)3 | 4.3 | 50 | 6000 |
| 37 | 114 | | | | |
| Comparative | Compound A | Ir(ppy)₃ | 5.0 | 38 | 6000 |
| Example 1 | | | | | |
| Comparative | Compound B | Ir(ppy)₃ | 5.1 | 29 | 6000 |
| Example 2 | | | | | |
| Comparative | Compound C | Ir(ppy)₃ | 4.8 | 34 | 6000 |
| Example 3 | | | | | |
| Comparative | Compound D | Ir(ppy)₃ | 4.8 | 31 | 6000 |
| Example 4 | | | | | |

Referring to Table 5, the organic light-emitting devices of Examples 1, 2, ad-1 to ad-17, and ad-21 to ad-37 were found to have lower driving voltages and higher current efficiencies, as compared to those of the organic light-emitting devices of Comparative Examples 1 to 4.

TABLE 6

| | | | | | | | T95 |
|---|---|---|---|---|---|---|---|
| | | | | Driving | Current | | Life |
| | The first | The second | | voltage | efficiency | Luminance | span |
| | host | host | Dopant | (V) | (cd/A) | (cd/m²) | (hr) |
| Example | Compound | Compound | Ir(ppy)3 | 4.2 | 54 | 6000 | 81 |
| ad-38 | a-70 | A1 | | | | | |
| Example | Compound | Compound | Ir(ppy)3 | 4.3 | 52 | 6000 | 82 |
| ad-39 | a-70 | A2 | | | | | |
| Example | Compound | Compound | Ir(ppy)3 | 4.4 | 53 | 6000 | 80 |
| ad-40 | a-70 | A5 | | | | | |
| Example | Compound | Compound | Ir(ppy)3 | 4.3 | 51 | 6000 | 83 |
| ad-41 | a-70 | A15 | | | | | |
| Example | Compound | Compound | Ir(ppy)3 | 4.0 | 55 | 6000 | 85 |
| ad-42 | a-70 | A17 | | | | | |
| Example | Compound | Compound | Ir(ppy)3 | 4.3 | 54 | 6000 | 84 |
| ad-43 | a-70 | A63 | | | | | |
| Example | Compound | Compound | Ir(ppy)3 | 4.2 | 55 | 6000 | 85 |
| ad-44 | a-70 | A64 | | | | | |
| Example | Compound | Compound | Ir(ppy)3 | 4.4 | 52 | 6000 | 80 |
| ad-45 | a-70 | B2 | | | | | |
| Example | Compound | Compound | Ir(ppy)3 | 4.1 | 56 | 6000 | 84 |
| ad-46 | a-40 | A17 | | | | | |
| Example | Compound | Compound | Ir(ppy)3 | 4.0 | 55 | 6000 | 84 |
| ad-47 | a-71 | A17 | | | | | |
| Example | Compound | Compound | Ir(ppy)3 | 4.1 | 53 | 6000 | 80 |
| ad-48 | a-74 | A17 | | | | | |
| Example | Compound | Compound | Ir(ppy)3 | 4.2 | 56 | 6000 | 85 |
| ad-49 | a-75 | A17 | | | | | |
| Example | Compound | Compound | Ir(ppy)3 | 4.3 | 55 | 6000 | 84 |
| ad-50 | a-82 | A17 | | | | | |
| Example | Compound | Compound | Ir(ppy)3 | 4.2 | 55 | 6000 | 81 |
| ad-51 | a-84 | A17 | | | | | |
| Example | Compound | Compound | Ir(ppy)3 | 4.1 | 53 | 6000 | 83 |
| ad-52 | a-75 | A63 | | | | | |
| Example | Compound | Compound | Ir(ppy)3 | 4.0 | 57 | 6000 | 88 |
| ad-53 | a-75 | A64 | | | | | |
| Example | Compound | Compound | Ir(ppy)3 | 4.2 | 55 | 6000 | 85 |
| ad-54 | e-75 | A17 | | | | | |
| Example | Compound | Compound | Ir(ppy)3 | 4.0 | 54 | 6000 | 84 |
| ad-55 | e-114 | A17 | | | | | |
| Example | Compound | Compound | Ir(ppy)3 | 4.1 | 56 | 6000 | 87 |
| ad-56 | f-75 | A17 | | | | | |
| Example | Compound | Compound | Ir(ppy)3 | 4.1 | 55 | 6000 | 85 |
| ad-57 | f-114 | A17 | | | | | |
| Example | Compound | Compound | Ir(ppy)3 | 4.0 | 55 | 6000 | 86 |
| ad-58 | e-75 | A64 | | | | | |
| Example | Compound | Compound | Ir(ppy)3 | 4.1 | 55 | 6000 | 85 |
| ad-59 | e-114 | A64 | | | | | |
| Example | Compound | Compound | Ir(ppy)3 | 4.1 | 57 | 6000 | 88 |
| ad-60 | f-75 | A64 | | | | | |
| Example | Compound | Compound | Ir(ppy)3 | 3.9 | 54 | 6000 | 86 |
| ad-61 | f-114 | A64 | | | | | |

Referring to the Table 6, the organic light-emitting devices of Example ad-38 to ad-61 showed a low driving voltage, high efficiency and a long life-span compared with the organic light-emitting devices of Comparative Examples 1 to 4.

TABLE 7

| | | | | | 90% Life |
|---|---|---|---|---|---|
| | | | | Current | span (h) |
| | Emitting | Driving | | efficiency | At 5000 |
| No. | Layer | voltage (V) | EL color | (cd/A) | cd/m² |
| Comparative | CBP | 6.5 | red | 5.8 | 20 |
| Example | | | | | |
| ad−1 | | | | | |
| Example | b-116 | 5.0 | red | 12.7 | 60 |
| ad-62 | | | | | |
| Example | a-108 | 5.1 | red | 13.4 | 75 |
| ad-63 | | | | | |

Referring to the Table 7, the organic light-emitting devices of Example ad-62 and ad-63 showed improved characteristics in terms of driving voltage, luminous efficiency and/or power efficiency compared with the organic light-emitting device of Comparative Example ad-1.

### Manufacture of Organic Light-Emitting Device (ETB Device)

### Example ad-64

A glass substrate coated with a 1500 Å-thick ITO (Indium tin oxide) thin film was washed with distilled water/ultrasonic wave. The washed glass substrate was ultrasonic wave-washed with a solvent such as isopropyl alcohol, acetone, methanol and the like, dried, delivered to a plasma cleaner, cleaned by using an oxygen plasma therein, cleaned it for 10 minutes, and delivered to a vacuum depositor. This obtained ITO transparent electrode was used as an anode, and a 1400 Å-thick hole injection and transport layer was formed thereon by depositing HT13. Subsequently, on the hole transport layer (HTL), a 200 Å-thick emission layer was formed by doping BH113 and BD370 made by SFC Co. Ltd. as a blue florescent light-emitting host and dopant in an amount of 5 wt %. Then, on the emission layer, a 50 Å-thick electron transport auxiliary layer was formed by depositing the compound b-41 of Synthesis Example ad-18. On the electron transport auxiliary layer, a 310 Å-thick electron transport layer (ETL) was formed by vacuum-depositing tris(8-hydroxyquinoline) aluminum (Alq3), and a cathode was formed by sequentially vacuum-depositing 15 Å-thick Liq and 1200 Å-thick Al on the electron transport layer (ETL), manufacturing an organic light-emitting device.

The organic light-emitting device had a five organic thin film-layered structure, specifically

ITO/HT13 1400 Å//EML[BH113:BD370=95:5 wt %] 200 Å/compound b-4 150 Å/A1q3 310 Å/Liq15 Å/Al 1200 Å.

### Example ad-65

An organic light-emitting device was manufactured according to the same method as Example ad-64 except for using the compound b-71 of Synthesis Example ad-19 instead of the compound b-41 of Example ad-42.

### Example ad-66

An organic light-emitting device was manufactured according to the same method as Example ad-64 except for using the compound a-40 of Synthesis Example ad-2 instead of the compound b-41 of Example ad-42.

### Example ad-67

An organic light-emitting device was manufactured according to the same method as Example ad-64 except for using the compound a-70 of Synthesis Example ad-7 instead of the compound b-41 of Example ad-42.

### Example ad-68

An organic light-emitting device was manufactured according to the same method as Example ad-64 except for using the compound a-71 of Synthesis Example ad-8 instead of the compound b-41 of Example ad-42.

### Example ad-69

An organic light-emitting device was manufactured according to the same method as Example ad-64 except for using the compound a-74 of Synthesis Example ad-9 instead of the compound b-41 of Example ad-42.

### Example ad-70

An organic light-emitting device was manufactured according to the same method as Example ad-64 except for using the compound a-75 of Synthesis Example ad-10 instead of the compound b-41 of Example ad-42.

### Example ad-71

An organic light-emitting device was manufactured according to the same method as Example ad-64 except for using the compound a-82 of Synthesis Example ad-11 instead of the compound b-41 of Example ad-42.

### Example ad-72

An organic light-emitting device was manufactured according to the same method as Example ad-64 except for using the compound a-84 of Synthesis Example ad-12 instead of the compound b-41 of Example ad-42.

### Example ad-73

An organic light-emitting device was manufactured according to the same method as Example ad-64 except for using the compound a-74 of Synthesis Example ad-28 instead of the compound b-41 of Example ad-42.

### Example ad-74

An organic light-emitting device was manufactured according to the same method as Example ad-64 except for using the compound a-75 of Synthesis Example ad-29 instead of the compound b-41 of Example ad-42.

### Example ad-75

An organic light-emitting device was manufactured according to the same method as Example ad-64 except for using the compound a-114 of Synthesis Example ad-33 instead of the compound b-41 of Example ad-42.

### Example ad-76

An organic light-emitting device was manufactured according to the same method as Example ad-64 except for using the compound f-74 of Synthesis Example ad-36 instead of the compound b-41 of Example ad-42.

### Example ad-77

An organic light-emitting device was manufactured according to the same method as Example ad-64 except for using the compound f-75 of Synthesis Example ad-37 instead of the compound b-41 of Example ad-42.

### Example ad-78

An organic light-emitting device was manufactured according to the same method as Example ad-64 except for using the compound f-114 of Synthesis Example ad-41 instead of the compound b-41 of Example ad-42.

### Comparative Example ad-2

An organic light-emitting device was manufactured according to the same method as Example ad-64 except for using no electron transport auxiliary layer.

### Example ad-79

An organic light-emitting device was manufactured according to the same method as Example ad-64 except for forming an emission layer by forming a 1350 Å-thick hole injection and transport layer instead of the 1400 Å-thick hole injection and transport layer and a 50 Å-thick hole transport auxiliary layer by vacuum-depositing a compound P-5 on the hole transport layer (HTL) and then, a 50 Å-thick electron transport auxiliary layer by vacuum-depositing the compound a-46 of Synthesis Example ad-5 on the emission layer.

The organic light-emitting device has a six organic thin film-layered structure, specifically

a structure of ITO/HT13 1350 Å/P-5 50 Å/EML[BH113:BD370=95:5 wt %] 200 Å/compound a-46 50 Å/A1q3 310 Å/Liq 15 Å/Al 1200 Å.

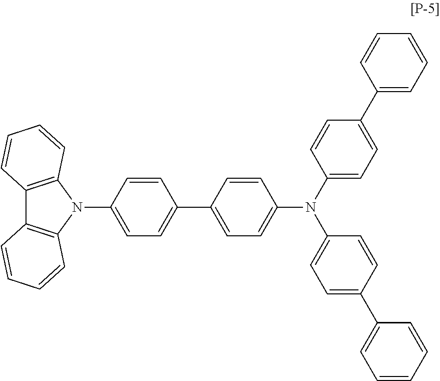 [SMILES: c1ccc(-c2ccc(N(c3ccc(-c4ccccc4)cc3)c3ccc(-c4ccc(-n5c6ccccc6c6ccccc65)cc4)cc3)cc2)cc1]

### Example ad-80

An organic light-emitting device was manufactured according to the same method as Example ad-79 except for using the compound of Synthesis Example ad-19 instead of the compound a-46 of Example ad-79.

### Comparative Example ad-3

An organic light-emitting device was manufactured according to the same method as Example ad-79 except for using no electron transport auxiliary layer.

### Evaluation Example 4

### Characteristics (II) of Organic Light-Emitting Device

Current density and luminance changes depending on a voltage, luminous efficiency and life-span of the organic light-emitting devices according to Examples ad-64 to ad-80, and Comparative Examples ad-2 and ad-3 were measured, and the results are provided in the following Tables 8 and 9.

A method of measuring (1) Current Density Change Depending on Voltage Change, (2) Luminance Change Depending on Voltage Change and (3) Luminous Efficiency are follows as the Evaluation Example 3.

Specifically, a life-span was measured as follows.

Life-Span

T97 life-spans of the organic light-emitting devices of Examples ad-64 to ad-80 and Comparative Examples ad-2 and ad-3 were measured as a time when their luminance decreased down to 97% relative to the initial luminance after emitting light with 750 cd/m² as the initial luminance (cd/m²) and measuring their luminance decrease depending on time with a Polanonix life-span measurement system.

TABLE 8

| | Electron | Color | T97 Life |
|---|---|---|---|
| | transport | Coordination | span(h) |
| Device | auxiliary layer | (x, y) | @750 nit |
| Example | Compound b- | (0.133, | 163 |
| ad-64 | 41 | 0.148) | |
| Example | Compound b- | (0.132, | 170 |
| ad-65 | 71 | 0.149) | |
| Example | Compound a- | (0.132, | 175 |
| ad-66 | 40 | 0.148) | |
| Example | Compound a- | (0.133, | 190 |
| ad-67 | 70 | 0.147) | |
| Example | Compound a- | (0.133, | 195 |
| ad-68 | 71 | 0.148) | |
| Example | Compound a- | (0.132, | 180 |
| ad-69 | 74 | 0.149) | |
| Example | Compound a- | (0.132, | 197 |
| ad-70 | 75 | 0.148) | |
| Example | Compound a- | (0.133, | 190 |
| ad-71 | 82 | 0.149) | |
| Example | Compound a- | (0.133, | 183 |
| ad-72 | 84 | 0.149) | |
| Example | Compound e- | (0.133, | 184 |
| ad-73 | 74 | 0.148) | |
| Example | Compound e- | (0.133, | 189 |
| ad-74 | 75 | 0.149) | |
| Example | Compound e- | (0.133, | 187 |
| ad-75 | 114 | 0.148) | |
| Example | Compound f- | (0.133, | 185 |
| ad-76 | 74 | 0.148) | |
| Example | Compound f- | (0.133, | 191 |
| ad-77 | 75 | 0.148) | |
| Example | Compound f- | (0.133, | 188 |
| ad-78 | 114 | 0.149) | |
| Comparative | Not used | (0.133, | 120 |
| Example | | 0.146) | |
| ad-2 | | | |

Referring to Table 8, the organic light-emitting devices according to Examples ad-64 to ad-78 showed an increased life-span compared with the organic light-emitting devices according to Comparative Example ad-2. Accordingly, the electron transport auxiliary layer turned out to improve life-span characteristics of the organic light-emitting device.

TABLE 9

| | Hole | Electron | | | | T97 Life |
|---|---|---|---|---|---|---|
| | transport | transport | Driving | Current | Color | span |
| | auxiliary | auxiliary | Voltage | efficiency | Coordination | (h)@750 |
| Device | layer | layer | (V) | (cd/A) | (x, y) | nit |
| Example | Compound | Compound | 4.28 | 7.4 | (0.136, 0.144) | 198 |
| ad-79 | P-5 | a-46 | | | | |
| Example | Compound | Compound | 4.32 | 7.2 | (0.135, 0.147) | 196 |
| ad-80 | P-5 | b-71 | | | | |
| Comparative | Compound | Not used | 5.02 | 6.8 | (0.133, 0.146) | 120 |
| Example | P-5 | | | | | |
| ad-3 | | | | | | |

Referring to Table 9, the organic light-emitting devices of Examples ad-79 and ad-80 showed excellent driving voltage, luminous efficiency and life-span characteristics compared with the organic light-emitting device of Comparative Example ad-3.

1. A condensed cyclic compound represented by Formula 1: 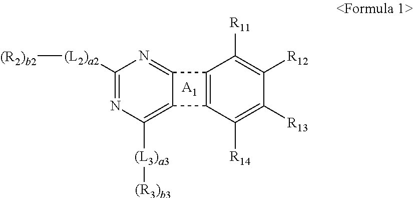
wherein, in Formula 1, ring A₁ is represented by Formula 1A, where X₁ is N-[(L₁)ₐ₁-(R₁)_{b1}], S, O, or Si(R₄)(R₅); 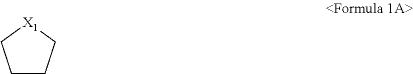
L₁ to L₃ are each independently selected from a substituted or unsubstituted C₆-C₆₀ arylene group,
a1 to a3 are each independently an integer selected from 0 to 5,
R₁ to R₅ are each independently selected from a hydrogen, a deuterium, a fluoro group (—F), a chloro group (—Cl), a bromo group (—Br), an iodo group (—I), a hydroxyl group, a substituted or unsubstituted C₁-C₆₀ alkyl group, a substituted or unsubstituted C₁-C₆₀ alkoxy group, a substituted or unsubstituted C₃-C₁₀ cycloalkyl group, a substituted or unsubstituted C₆-C₆₀ aryl group, a substituted or unsubstituted C₆-C₆₀ aryloxy group, a substituted or unsubstituted C₆-C₆₀ arylthio group, and a substituted or unsubstituted monovalent non-aromatic condensed polycyclic group,
wherein at least one of R₂ and R₃ is selected from a substituted or unsubstituted C₆-C₆₀ aryl group, and a substituted or unsubstituted monovalent non-aromatic condensed polycyclic group,
R₁₁ to R₁₄ are each independently selected from a hydrogen, a deuterium, —F, —Cl, —Br, —I, a hydroxyl group, a substituted or unsubstituted C₁-C₆₀ alkyl group, a substituted or unsubstituted C₁-C₆₀ alkoxy group, a C₃-C₁₀ cycloalkyl group, a C₆-C₆₀ aryl group, a C₆-C₆₀ aryloxy group, a C₆-C₆₀ arylthio group, and a monovalent non-aromatic condensed polycyclic group, and
b1 to b3 are each independently an integer selected from 1 to 3,
wherein R₃ is not a substituted or unsubstituted morpholinyl group;
when R₂ is a substituted or unsubstituted phenyl group, R₃ is selected from a hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted fluoranthenyl group, a substituted or unsubstituted triphenylenyl group, a substituted or unsubstituted pyrenyl group, a substituted or unsubstituted phenanthrenyl group, a substituted or unsubstituted fluorenyl group, and a substituted or unsubstituted chrysenyl group.

2. The condensed cyclic compound of claim 1, wherein the condensed cyclic compound is represented by one of Formulae 1-1 and 1-2: 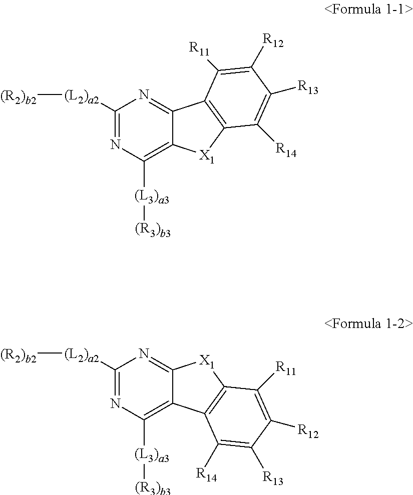
wherein, in Formulae 1-1 to 1-2, X₁, L₂, L₃, a2, a3, R₂, R₃, R₁₁ to R₁₄, b2 and b3 are the same as those defined in claim 1.

3. The condensed cyclic compound of claim 1, wherein X₁ is S or O.

4. The condensed cyclic compound of claim 1, wherein L₁ to L₃ are each independently represented by one of Formulae 2-1 to 2-15: 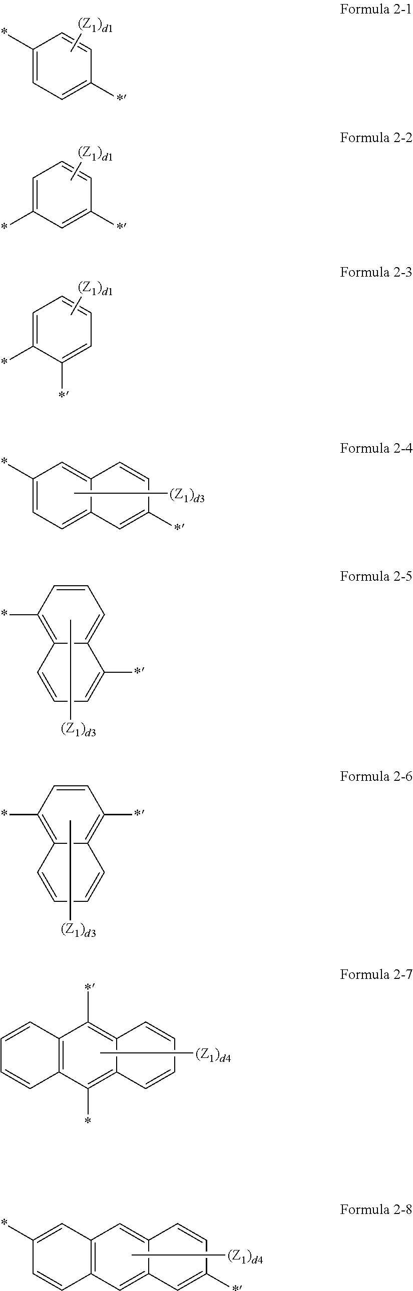 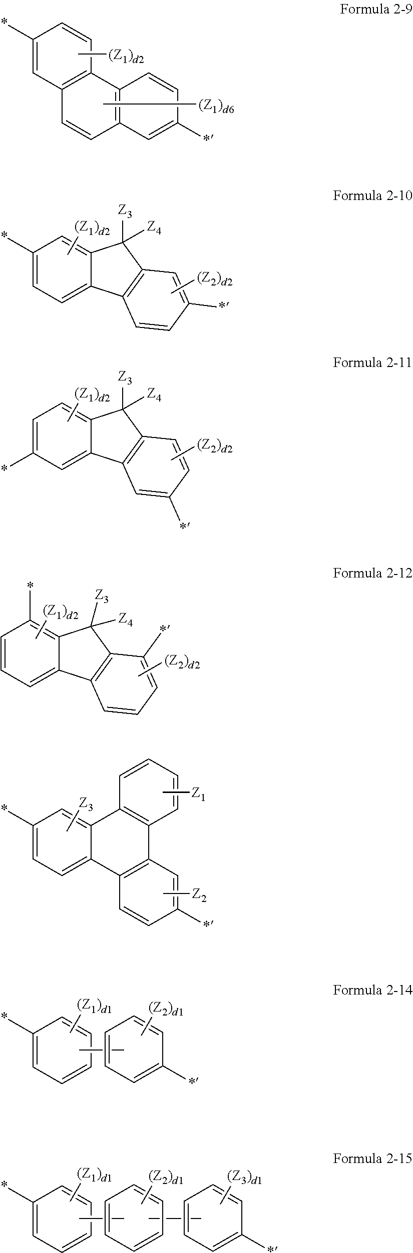
wherein, in Formulae 2-1 to 2-15,
Z₁ to Z₄ are each independently selected from a hydrogen, a deuterium, —F, —Cl, —Br, —I, a hydroxyl group, a C₁-C₂₀ alkyl group, a C₁-C₂₀ alkoxy group, a phenyl group, a biphenyl group, a terphenyl group, a quaterphenyl group, a naphthyl group, an anthracenyl group, a triphenylenyl group, a pyrenyl group, a phenanthrenyl group, a fluorenyl group, and a chrysenyl group;
d1 is an integer selected from 1 to 4;
d2 is an integer selected from 1 to 3;
d3 is an integer selected from 1 to 6;
d4 is an integer selected from 1 to 8;
d6 is an integer selected from 1 to 5; and
* and *′ are each independently a binding site with an adjacent atom.

5. The condensed cyclic compound of claim 1, wherein L₁ to L₃ are each independently represented by one of Formulae 3-1 to 3-37: 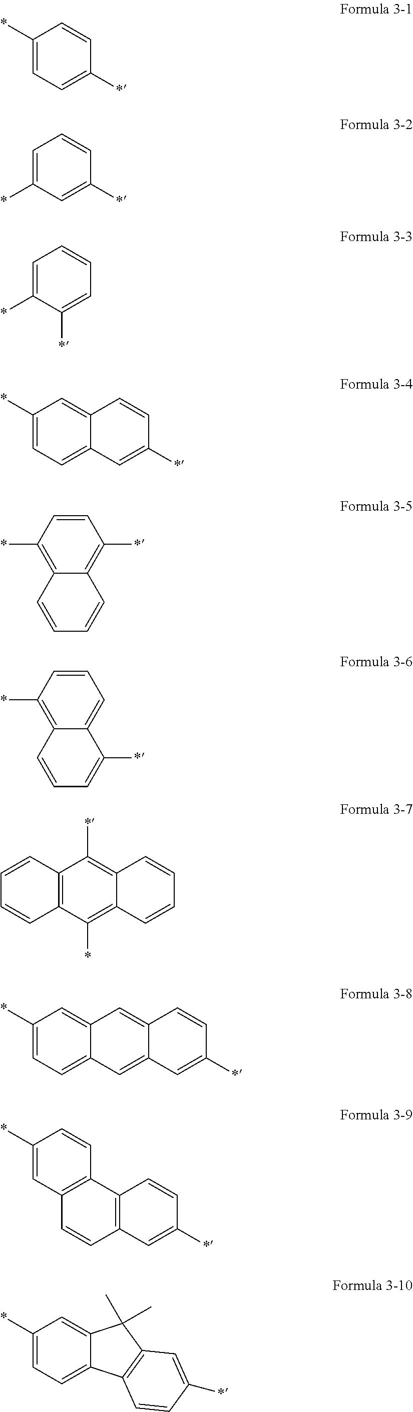 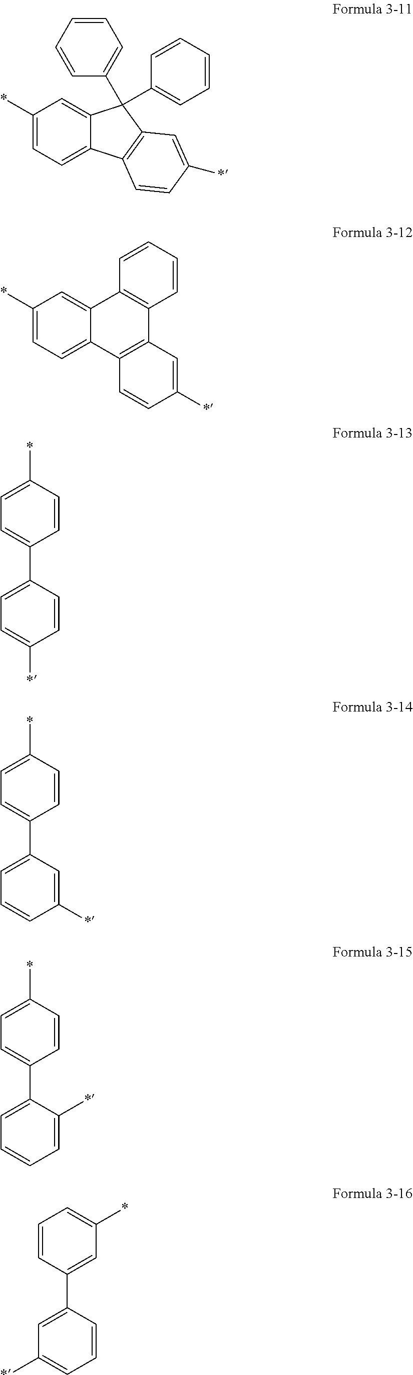 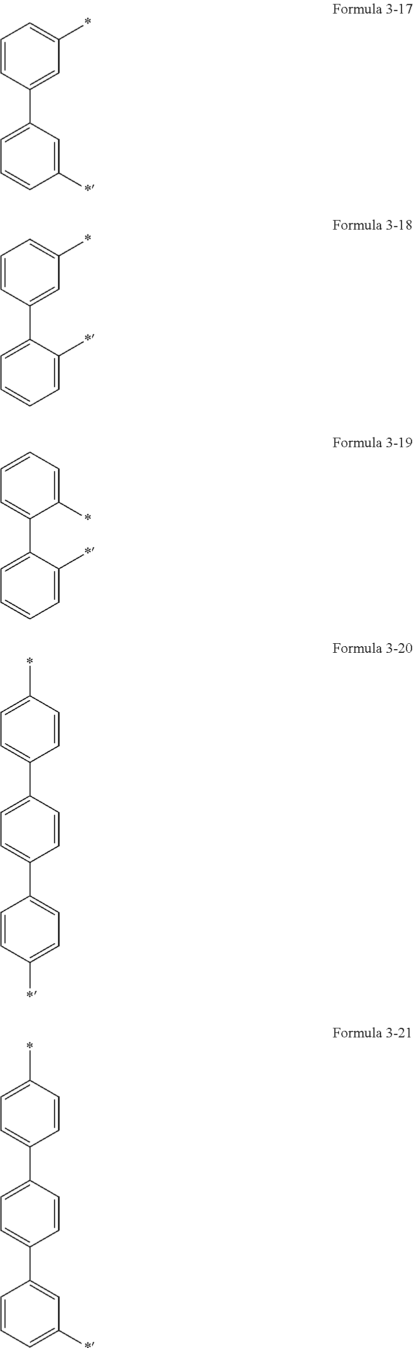 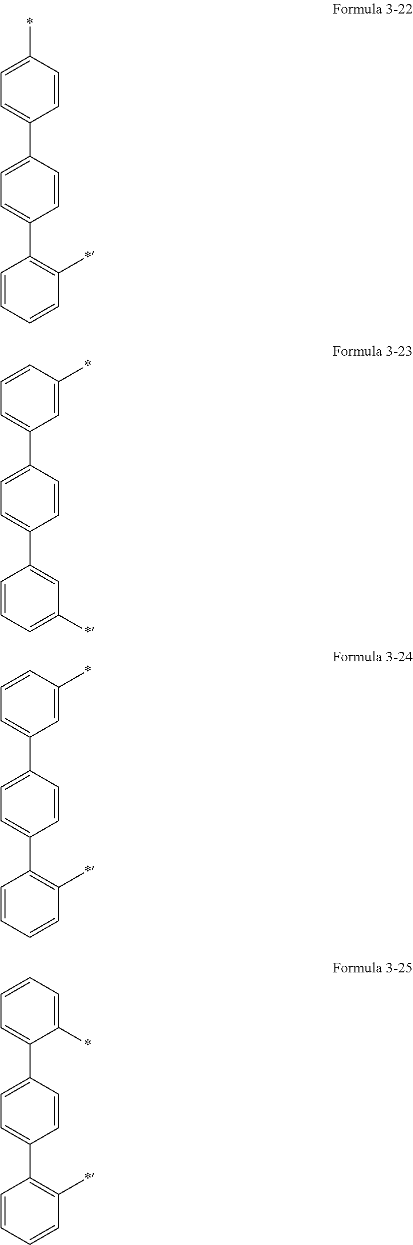 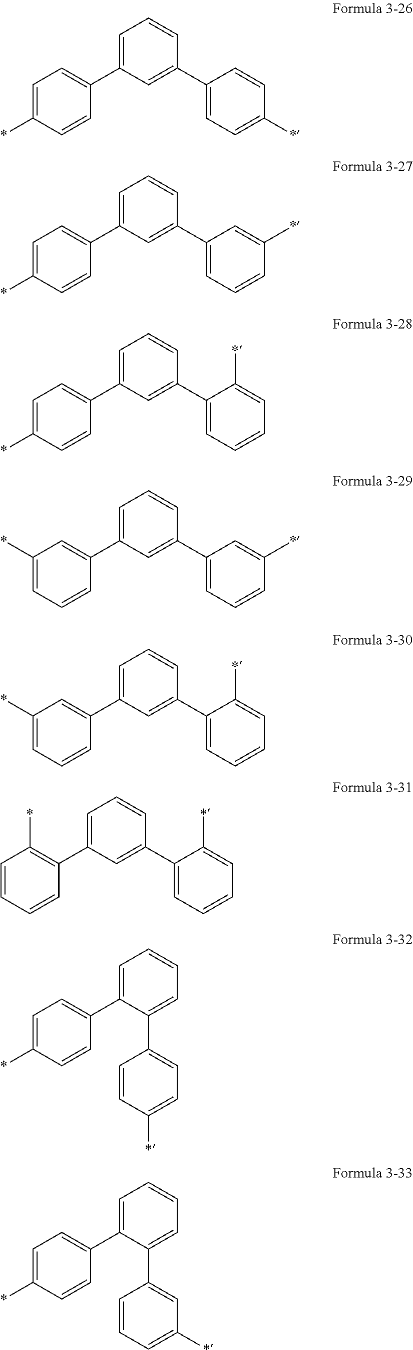 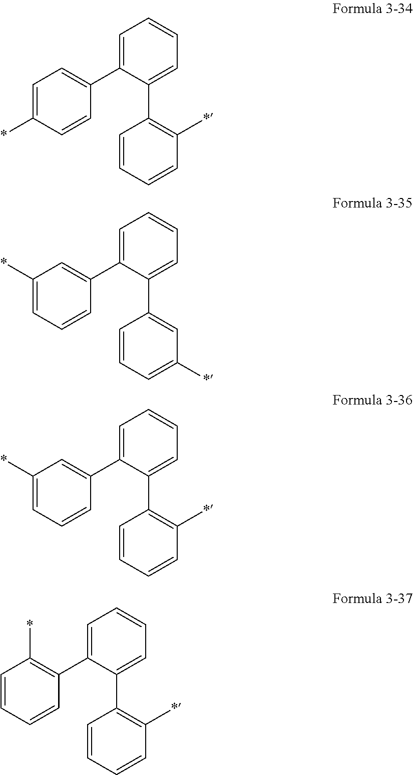
wherein, in Formulae 3-1 to 3-37,
* and *′ are each independently a binding site with an adjacent atom.

6. The condensed cyclic compound of claim 1, wherein R₁ to R₅ are each independently selected from
a hydrogen, a deuterium, —F, —Cl, —Br, —I, a hydroxyl group, a C₁-C₂₀ alkyl group, and a C₁-C₂₀ alkoxy group,
a C₁-C₂₀ alkyl group and a C₁-C₂₀ alkoxy group, each substituted with at least one of a deuterium atom, —F, —Cl, —Br, —I, and a hydroxyl group, and
a group represented by one of Formulae 4-1 to 4-5, and 4-34 to 4-37; and
i) at least one of R₂ and R₃, and ii) R₁ are each independently selected from a group represented by one of Formulae 4-1 to 4-5, and 4-34 to 4-37: 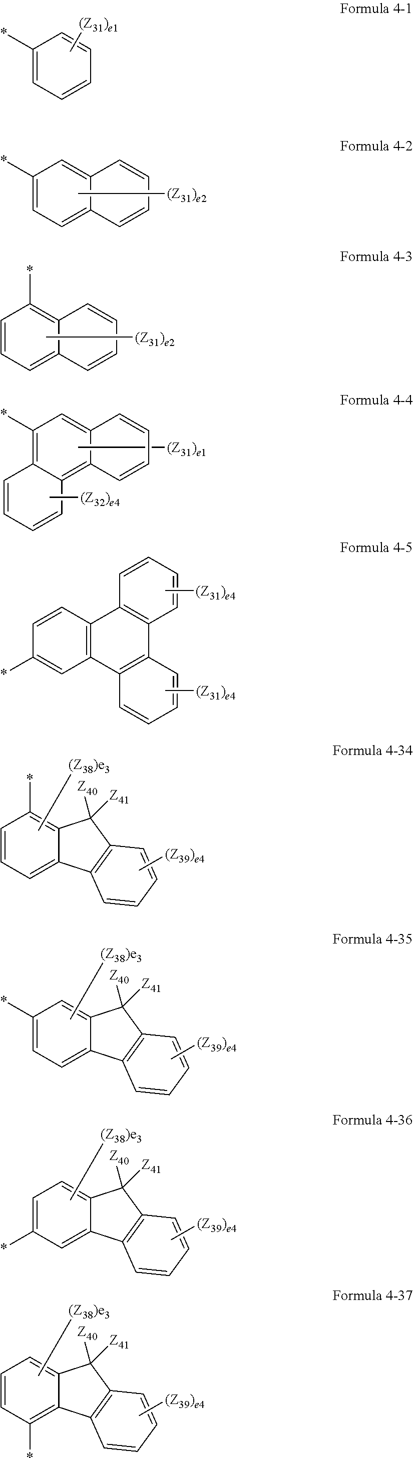
wherein, in Formulae 4-1 to 4-5, and 4-34 to 4-37,
Z₃₁ and Z₃₈ to Z₄₁ are each independently selected from a hydrogen, a deuterium, —F, —Cl, —Br, —I, a hydroxyl group, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a chrysenyl group, a biphenyl group, a terphenyl group, and a quaterphenyl group,
e1 is an integer selected from 1 to 5,
e2 is an integer selected from 1 to 7,
e3 is an integer selected from 1 to 3,
e4 is an integer selected from 1 to 4, and
* is a binding site with an adjacent atom.

7. The condensed cyclic compound of claim 1,
wherein X₁ is S or O,
R₁ to R₅ are each independently selected from
a hydrogen, a deuterium, —F, —Cl, —Br, —I, a hydroxyl group, a C₁-C₂₀ alkyl group, and a C₁-C₂₀ alkoxy group,
a C₁-C₂₀ alkyl group and a C₁-C₂₀ alkoxy group, each substituted with at least one of a deuterium atom, —F, —Cl, —Br, —I, and a hydroxyl group, and
a group represented by one of Formulae 4-1 to 4-5, and 4-34 to 4-37; and
at least one of R₂ and R₃ are each independently selected from a group represented by one of Formulae 4-1 to 4-5, and 4-34 to 4-37: 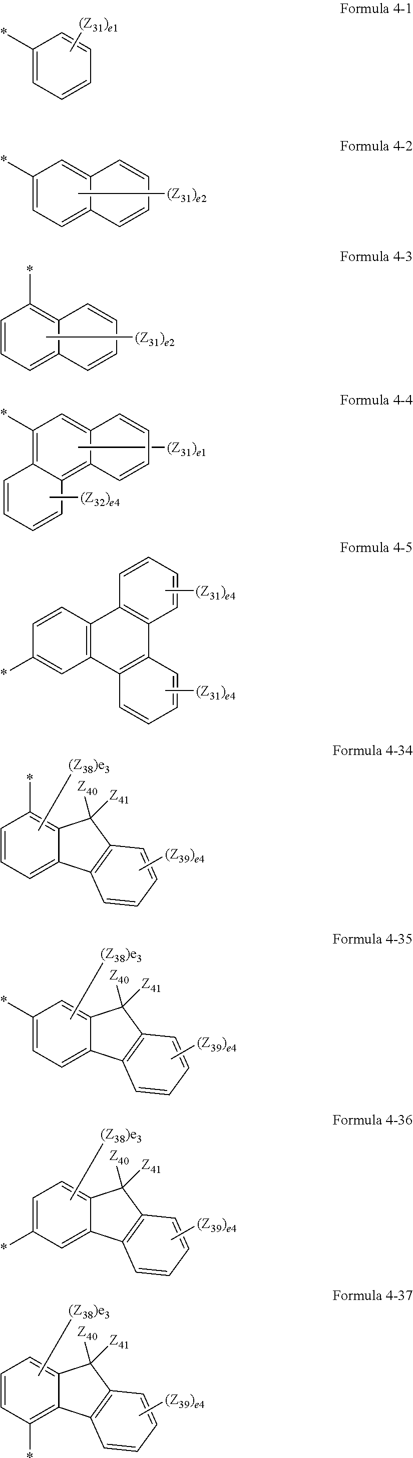
wherein, in Formulae 4-1 to 4-5, and 4-34 to 4-37,
Z₃₁ and Z₃₈ to Z₄₁ are each independently selected from a hydrogen, a deuterium, —F, —Cl, —Br, —I, a hydroxyl group, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a chrysenyl group, a biphenyl group, a terphenyl group, and a quaterphenyl group,
e1 is an integer selected from 1 to 5,
e2 is an integer selected from 1 to 7,
e3 is an integer selected from 1 to 3,
e4 is an integer selected from 1 to 4, and
* is a binding site with an adjacent atom.

8. The condensed cyclic compound of claim 1, wherein at least one of R₂ and R₃ is selected from
a phenyl group, a biphenyl group, a terphenyl group, a quaterphenyl group, a naphthyl group, phenanthrenyl group, an anthracenyl group, a fluoranthenyl group, and a triphenylenyl group, and
a biphenyl group, a terphenyl group, a quaterphenyl group, a naphthyl group, phenanthrenyl group, an anthracenyl group, a fluoranthenyl group, and a triphenylenyl group, each substituted with at least one selected from a deuterium, —F, —Cl, —Br, —I, a hydroxyl group, a C₁-C₂₀ alkyl group, a C₁-C₂₀ alkoxy group, a phenyl group, a naphthyl group, a phenalenyl group, a phenanthrenyl group, an anthracenyl group, a fluorantenyl group, a triphenylenyl group, a pyrenyl group, and a chrysenyl group.

9. The condensed cyclic compound of claim 1, wherein R₁₁ to R₁₄ are each independently selected from
a hydrogen, a deuterium, —F, —Cl, —Br, —I, a hydroxyl group, a C₁-C₂₀ alkyl group, and a C₁-C₂₀ alkoxy group, and
a phenyl group, a biphenyl group, a terphenyl group, a quaterphenyl group, a naphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, and a chrysenyl group.

10. The condensed cyclic compound of claim 1, wherein R₁ to R₅ are each independently selected from
a hydrogen, a deuterium, —F, —Cl, —Br, —I, a hydroxyl group, a C₁-C₂₀ alkyl group, and a C₁-C₂₀ alkoxy group,
a C₁-C₂₀ alkyl group and a C₁-C₂₀ alkoxy group, each substituted with at least one of a deuterium atom, —F, —Cl, —Br, —I, and a hydroxyl group,
a group represented by one of Formulae 5-1 to 5-9, 5-18 to 5-21, and 5-45 to 5-66, and wherein R₁₁ to R₁₄ are each independently selected from
a hydrogen, a deuterium, —F, —Cl, —Br, —I, a hydroxyl group, a C₁-C₂₀ alkyl group, and a C₁-C₂₀ alkoxy group, and
a group represented by one of Formulae 5-1 to 5-9, 5-18 to 5-21, and 5-45 to 5-66: 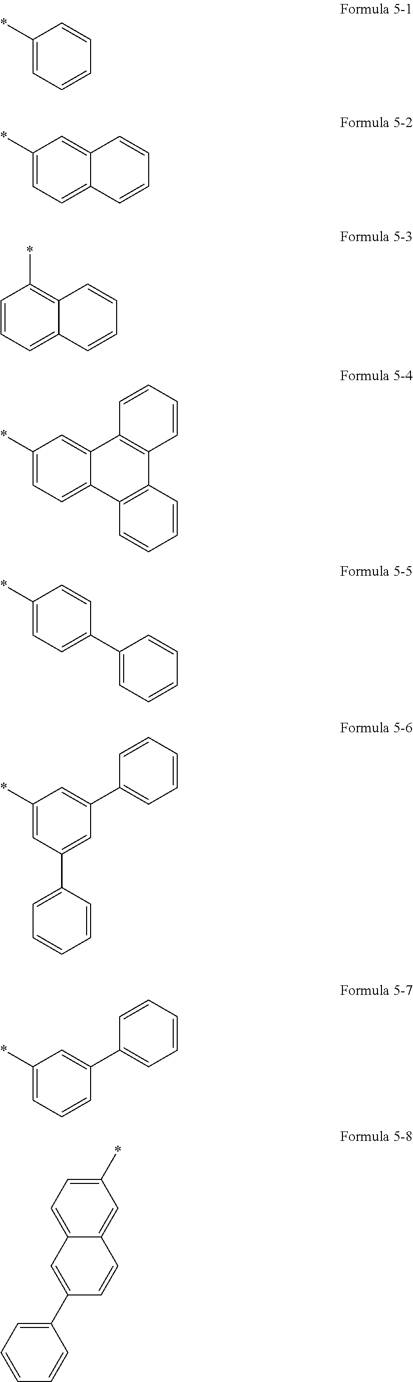 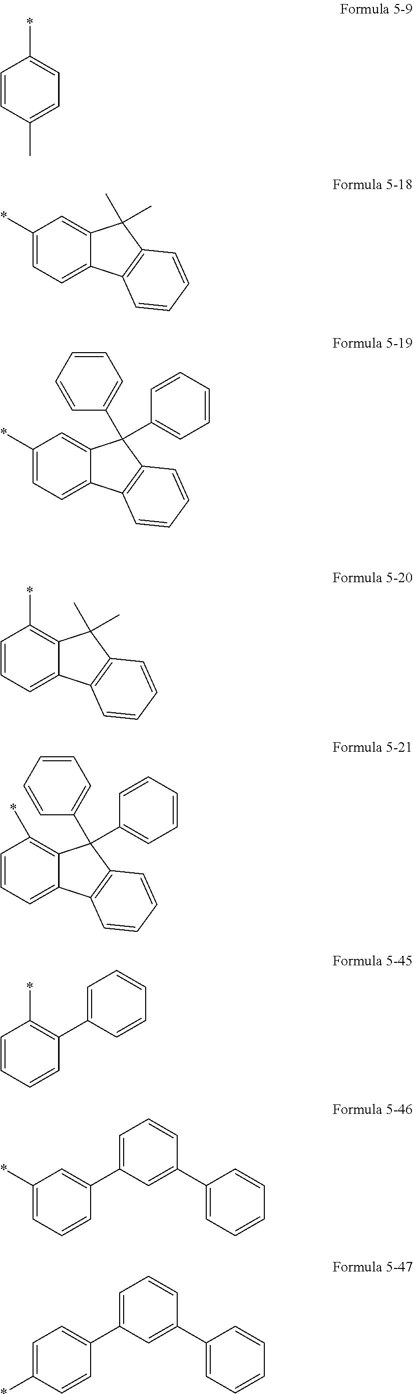 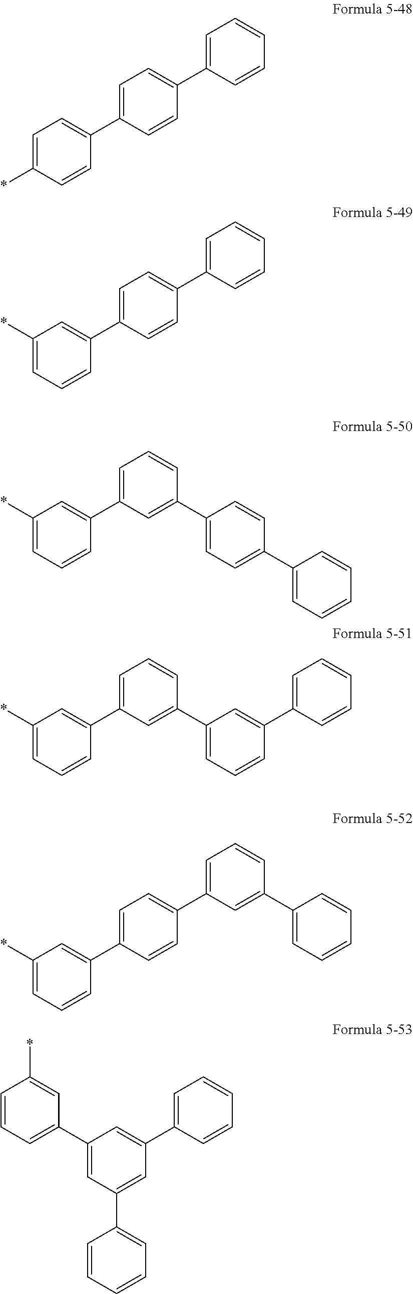 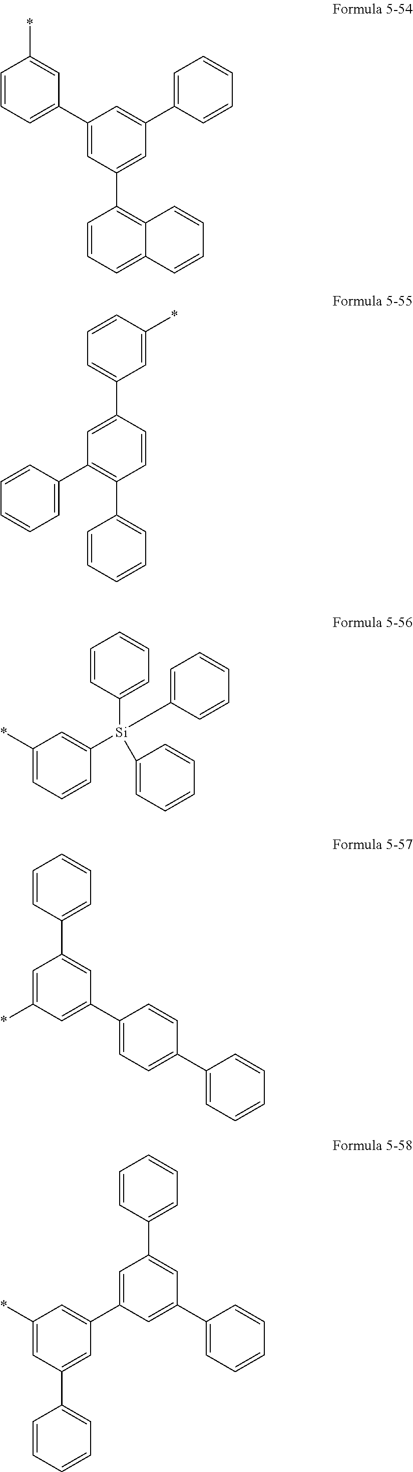 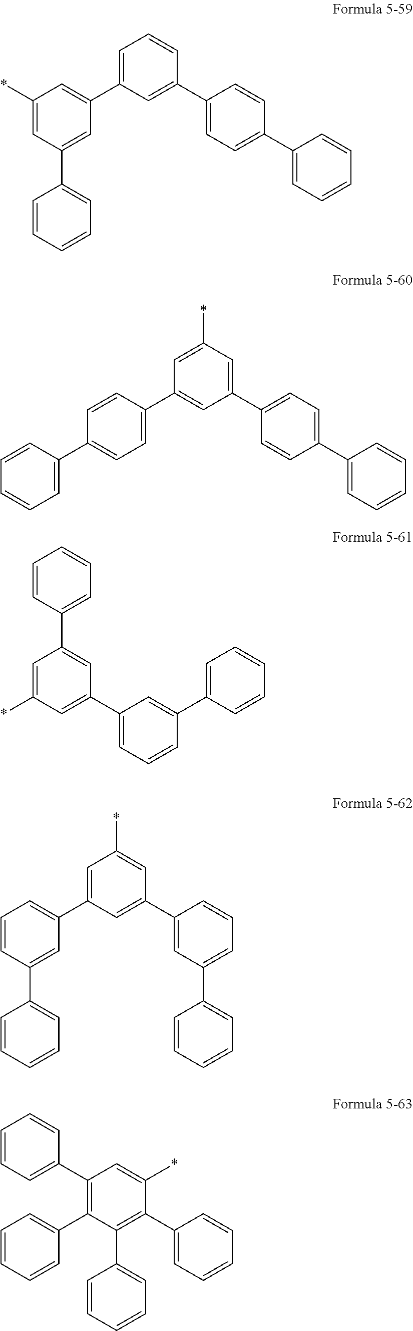 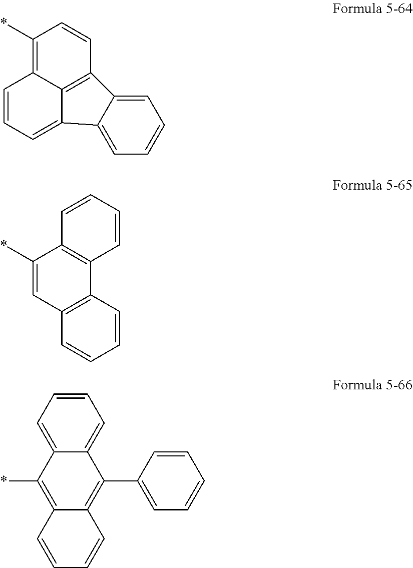
wherein, in Formulae 5-1 to 5-9, 5-18 to 5-21, and 5-45 to 5-66,
* is a binding site with an adjacent atom.

11. The condensed cyclic compound of claim 1,
wherein X₁ is S or O,
R₁ to R₅ are each independently selected from
a hydrogen, a deuterium, —F, —Cl, —Br, —I, a hydroxyl group, a C₁-C₂₀ alkyl group, and a C₁-C₂₀ alkoxy group,
a C₁-C₂₀ alkyl group and a C₁-C₂₀ alkoxy group, each substituted with at least one of a deuterium atom, —F, —Cl, —Br, —I, and a hydroxyl group, and
a group represented by one of Formulae 5-1 to 5-9, 5-18 to 5-21, and 5-45 to 5-66; and
at least one of R₂ and R₃ are each independently selected from a group represented by one of Formulae 5-1 to 5-9, 5-18 to 5-21, and 5-45 to 5-66,
wherein R₁₁ to R₁₄ are each independently selected from
a hydrogen, a deuterium, —F, —Cl, —Br, —I, a hydroxyl group, a C₁-C₂₀ alkyl group, and a C₁-C₂₀ alkoxy group, and
a group represented by one of Formulae 5-1 to 5-9, 5-18 to 5-21, and 5-45 to 5-66:

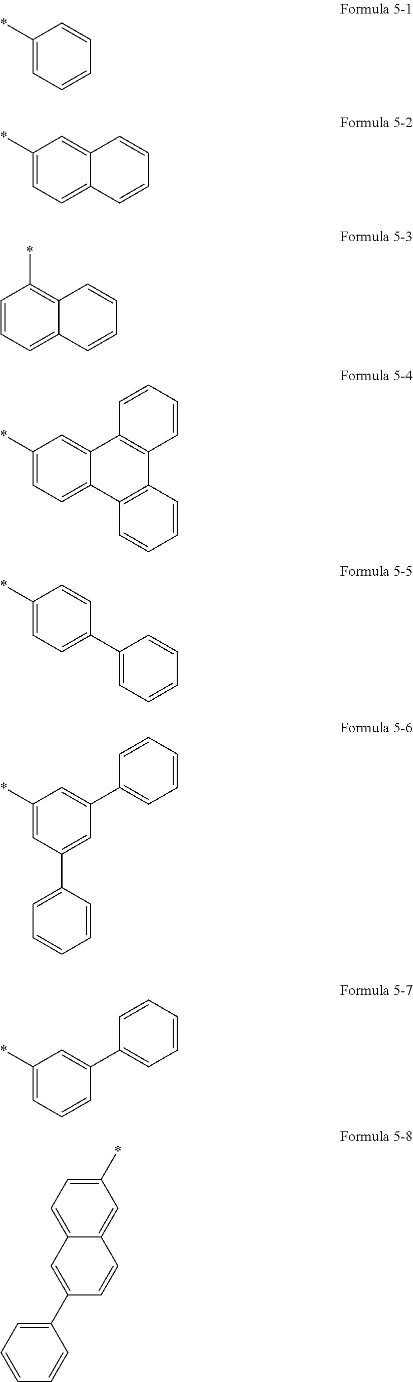

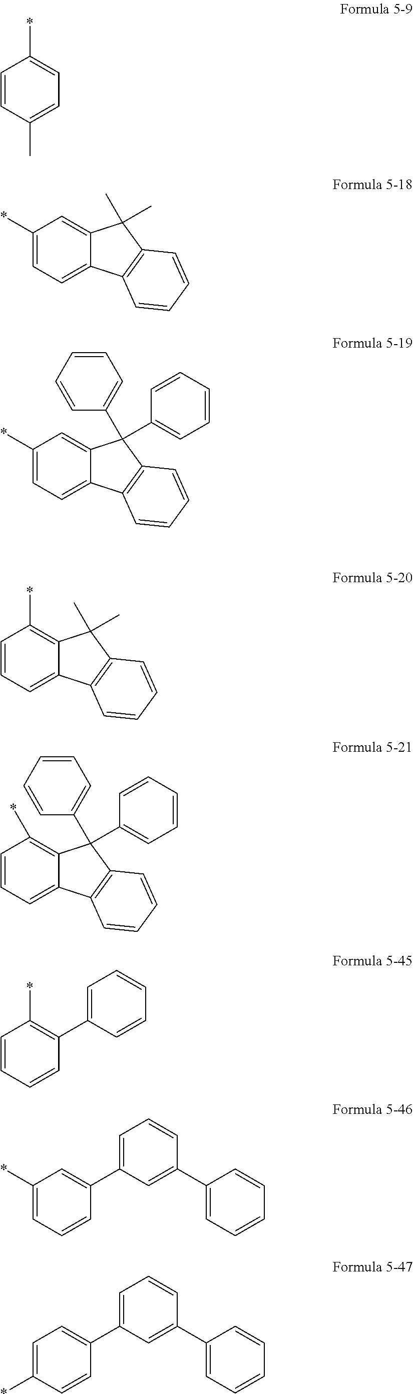

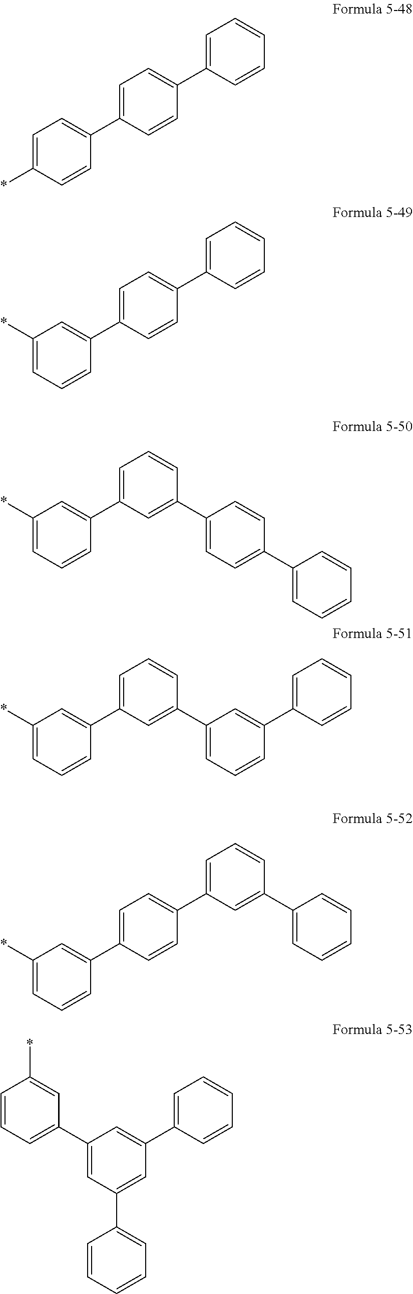

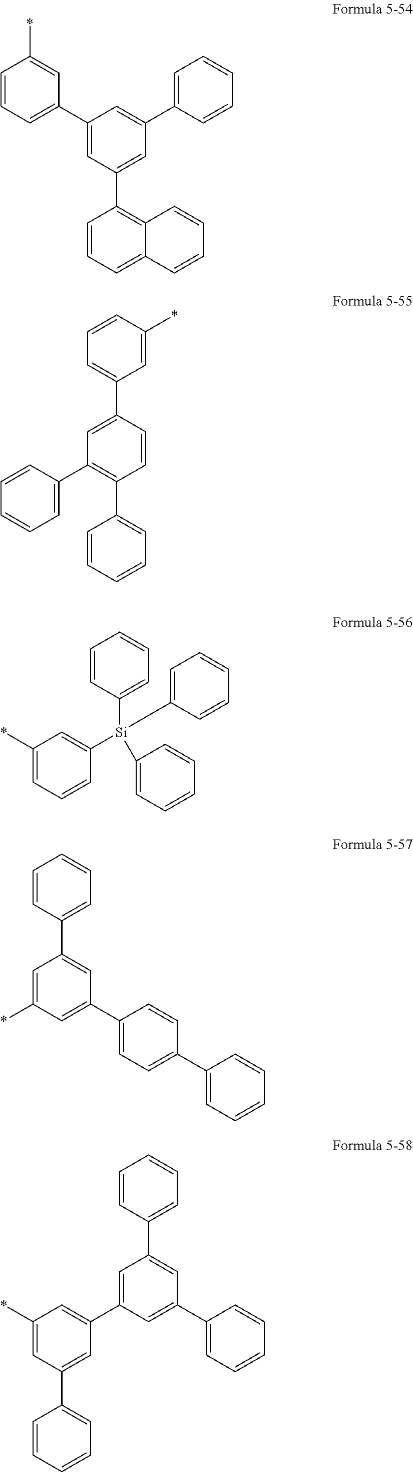

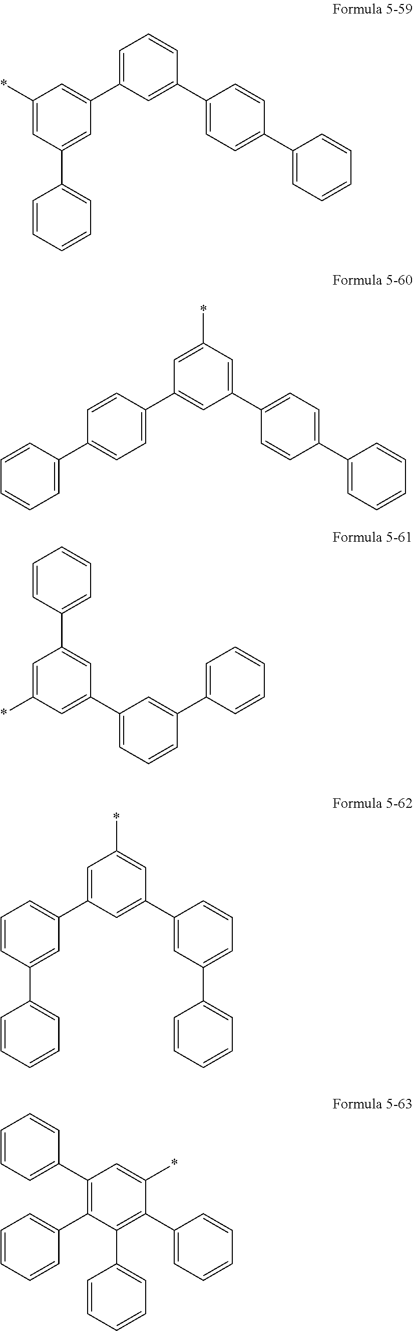

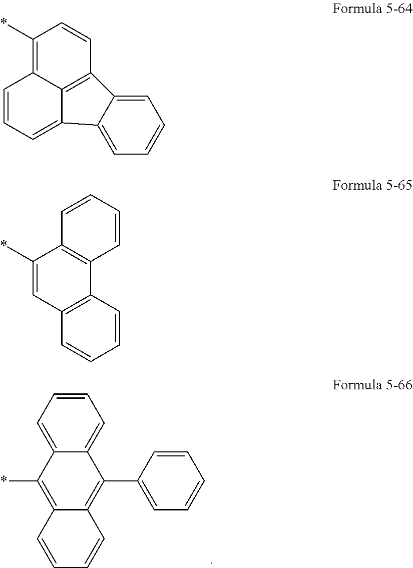

12. The condensed cyclic compound of claim 1, wherein the condensed cyclic compound of Formula 1 is one of Compounds listed following Group 1:

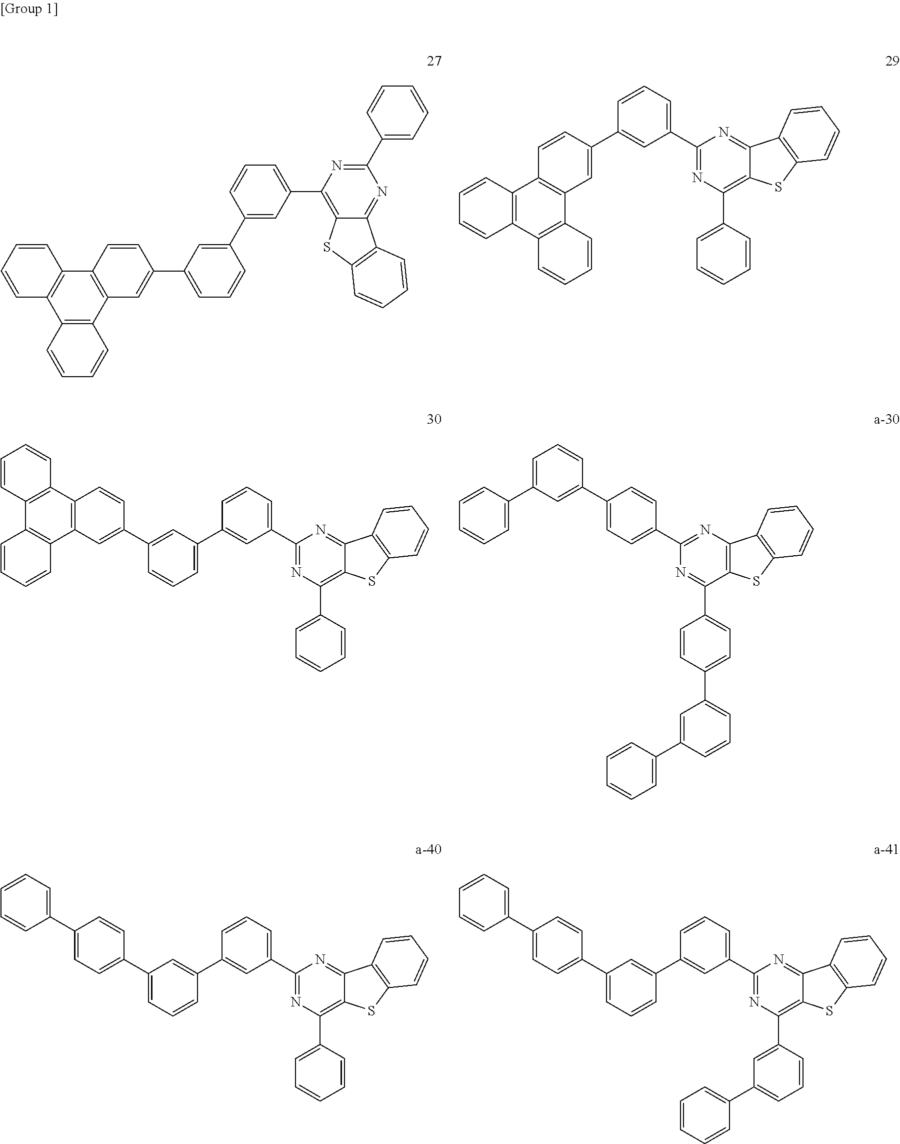

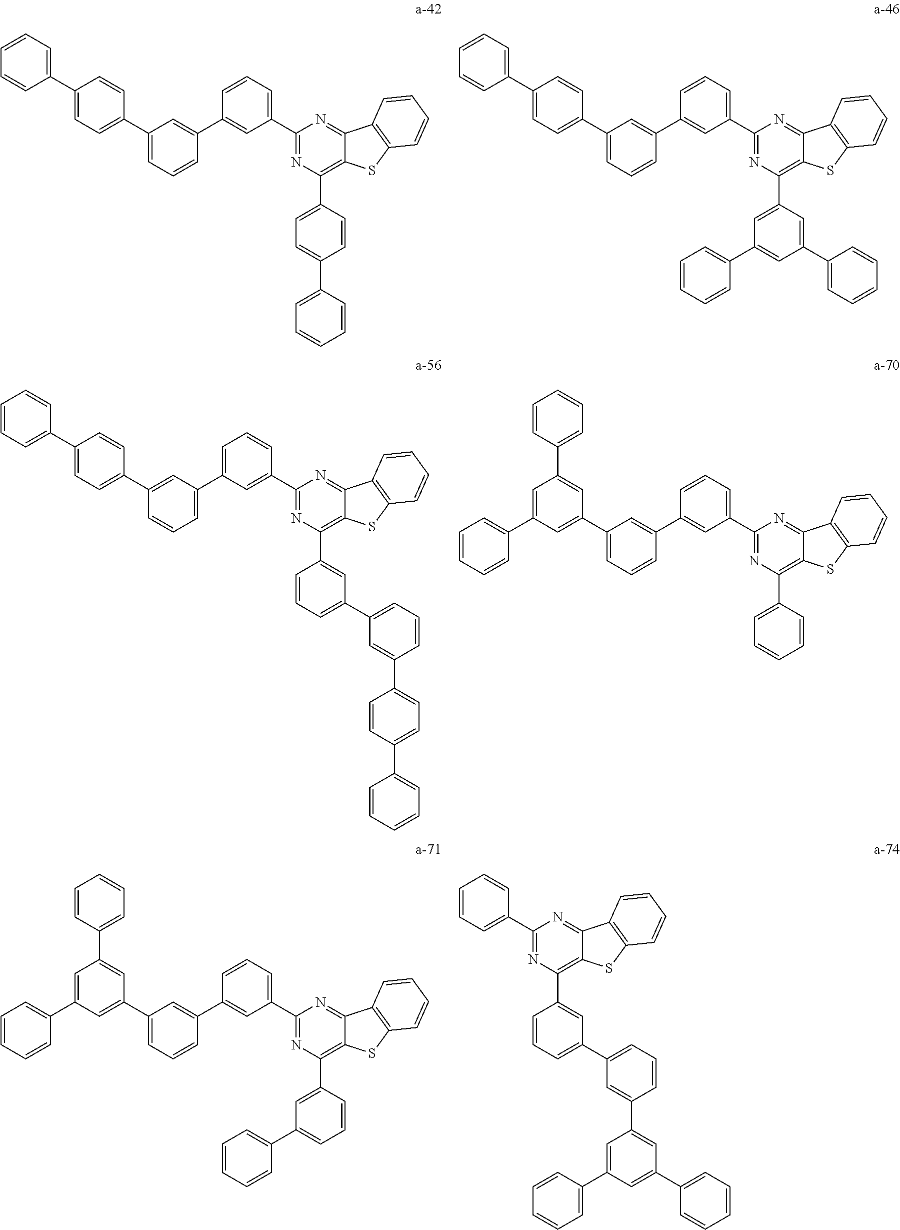

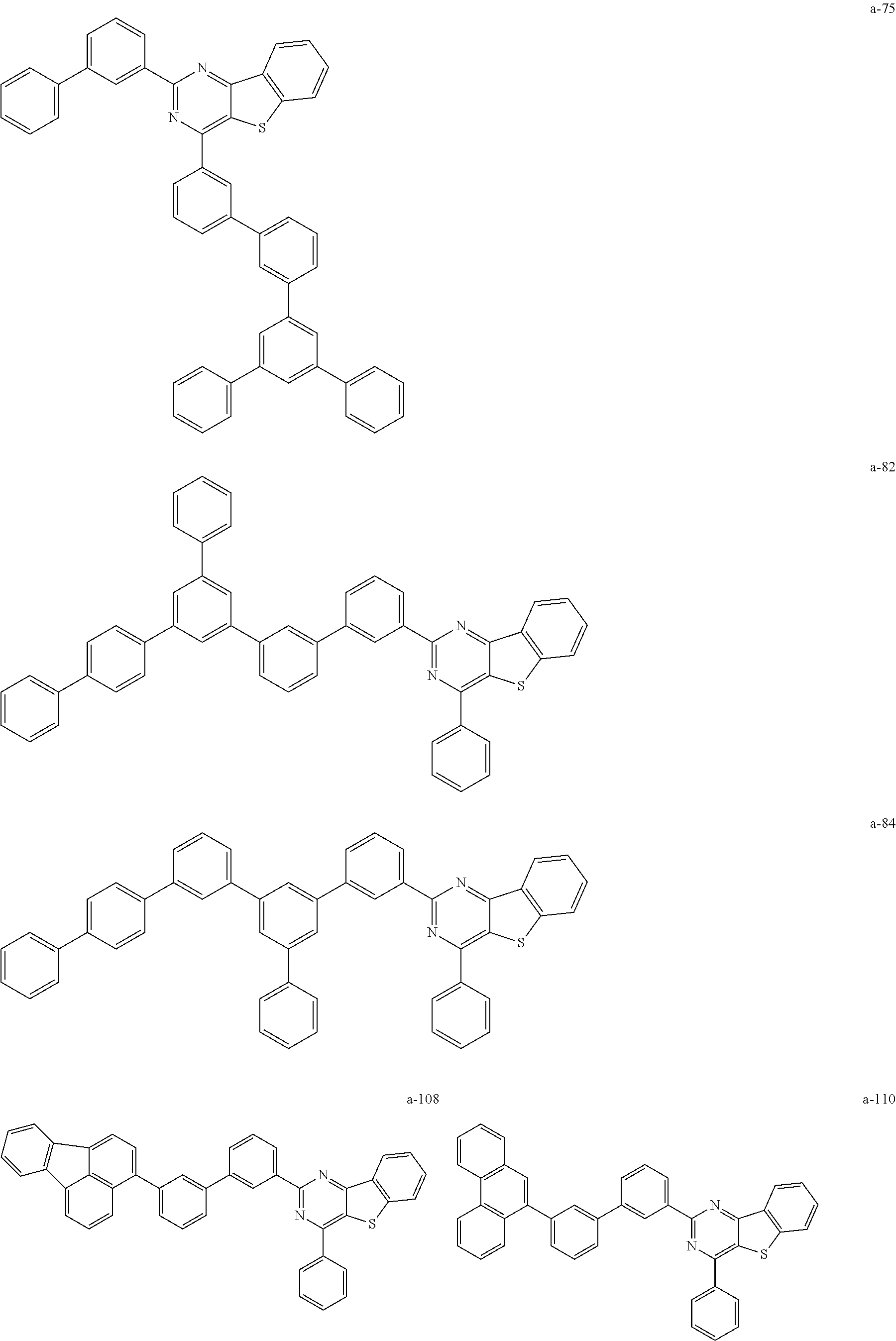

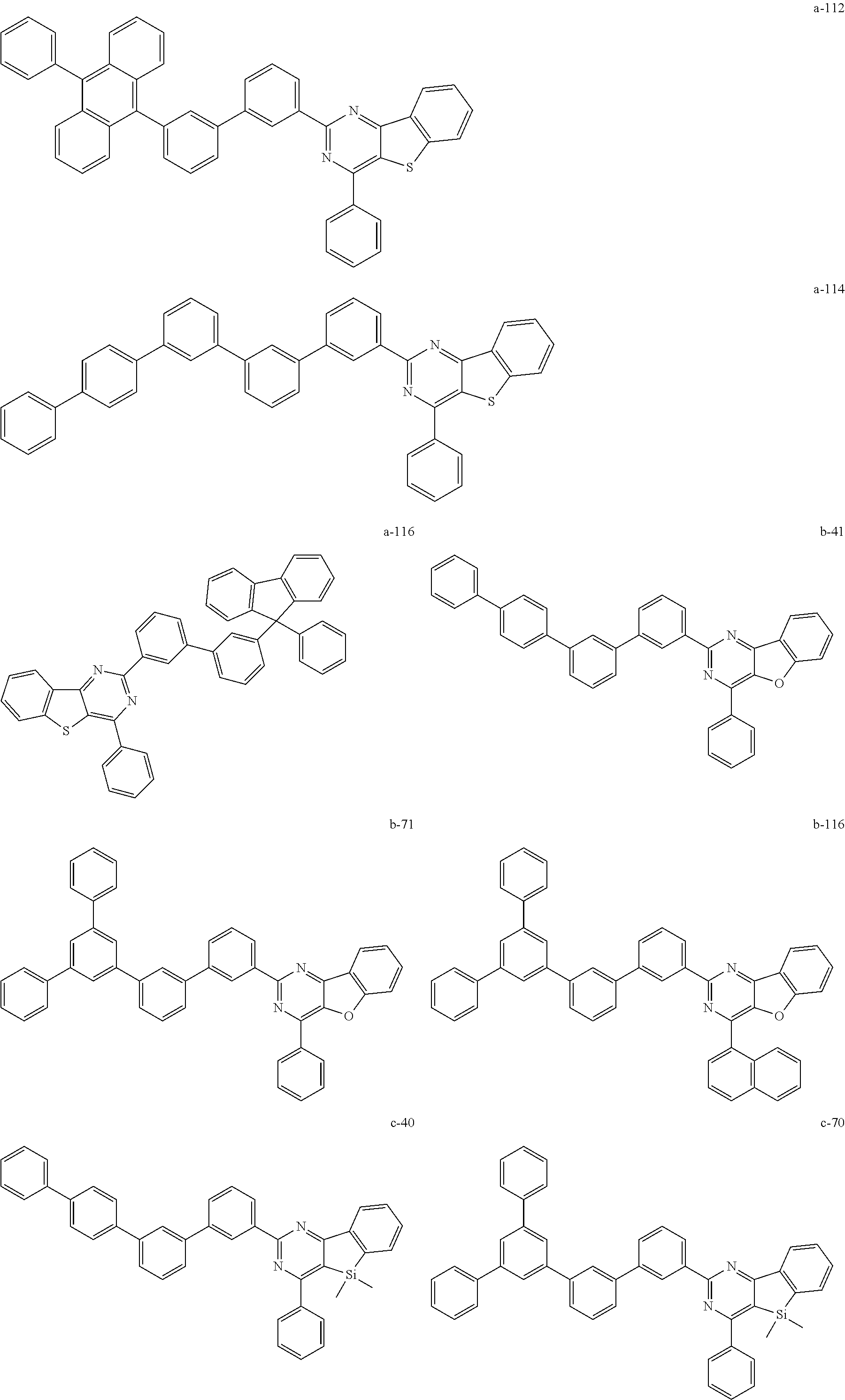

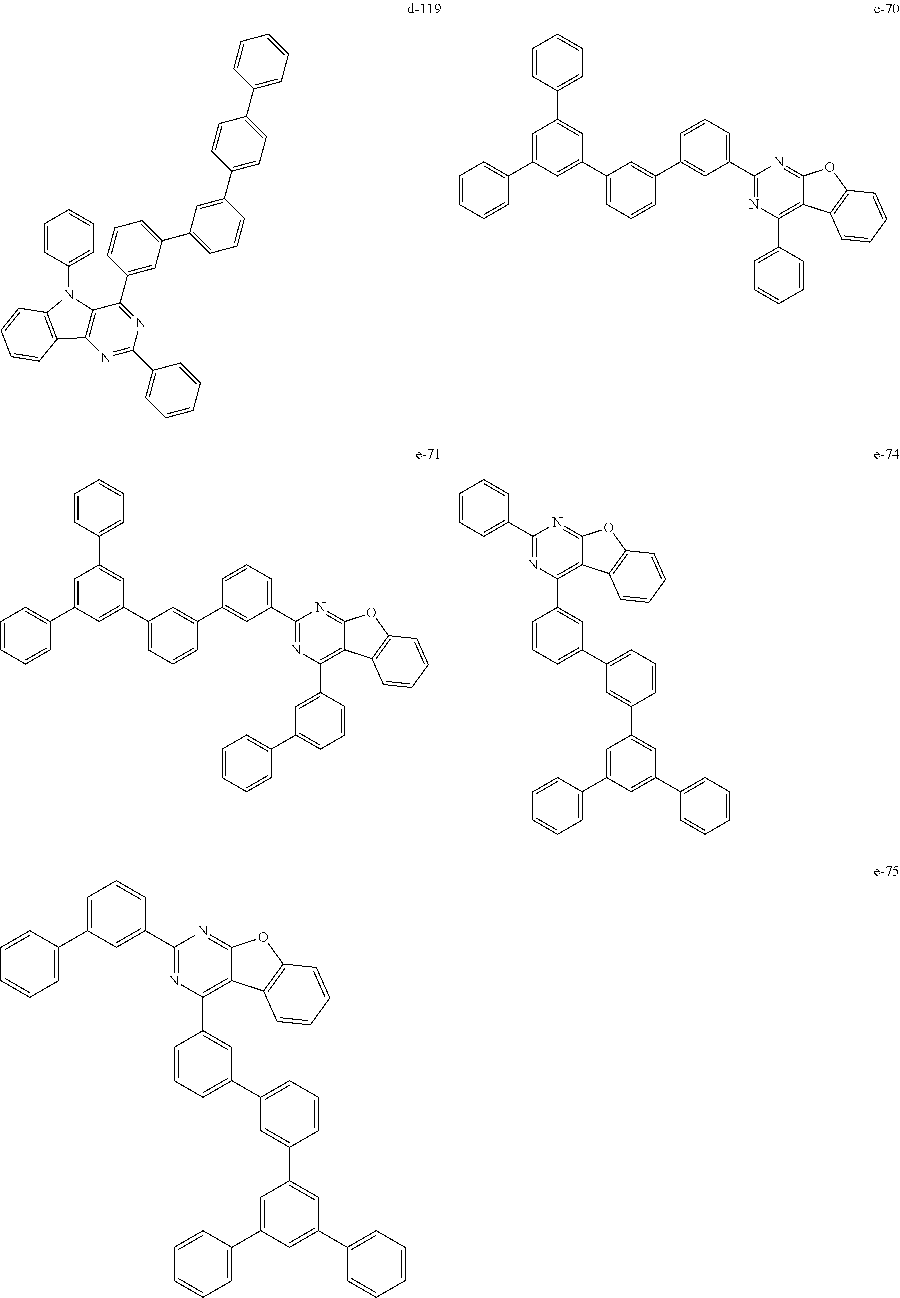 [SMILES: c1ccc(-c2ccc(-c3cccc(-c4cccc(-c5nc(-c6ccccc6)nc6c7ccccc7n(-c7ccccc7)c56)c4)c3)cc2)cc1 ; c1ccc(-c2cc(-c3ccccc3)cc(-c3cccc(-c4cccc(-c5nc(-c6ccccc6)c6c(n5)oc5ccccc56)c4)c3)c2)cc1 ; c1ccc(-c2cc(-c3ccccc3)cc(-c3cccc(-c4cccc(-c5nc(-c6cccc(-c7ccccc7)c6)c6c(n5)oc5ccccc56)c4)c3)c2)cc1 ; c1ccc(-c2cc(-c3ccccc3)cc(-c3cccc(-c4cccc(-c5nc(-c6ccccc6)nc6oc7ccccc7c56)c4)c3)c2)cc1 ; c1ccc(-c2cc(-c3ccccc3)cc(-c3cccc(-c4cccc(-c5nc(-c6cccc(-c7ccccc7)c6)nc6oc7ccccc7c56)c4)c3)c2)cc1]

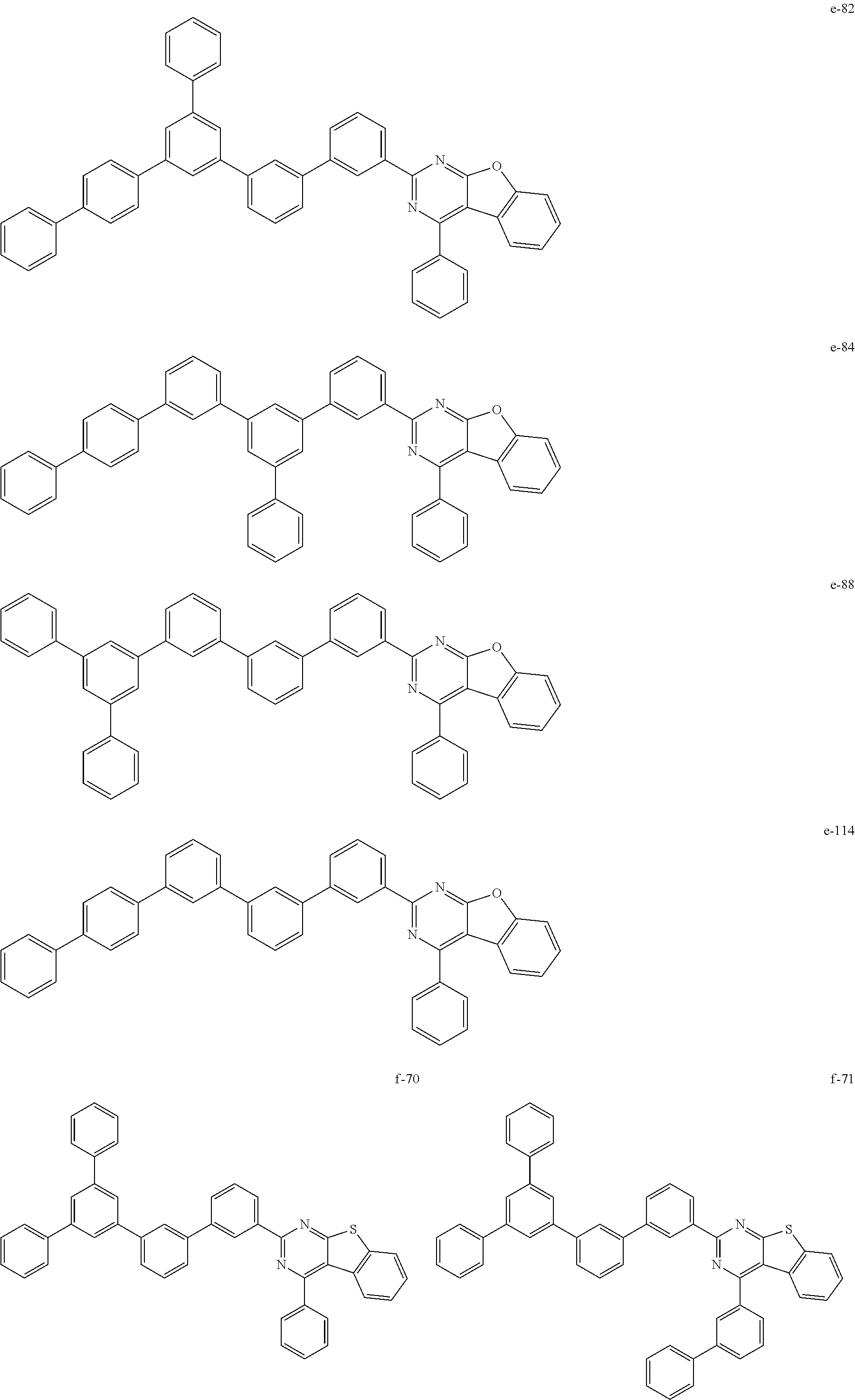

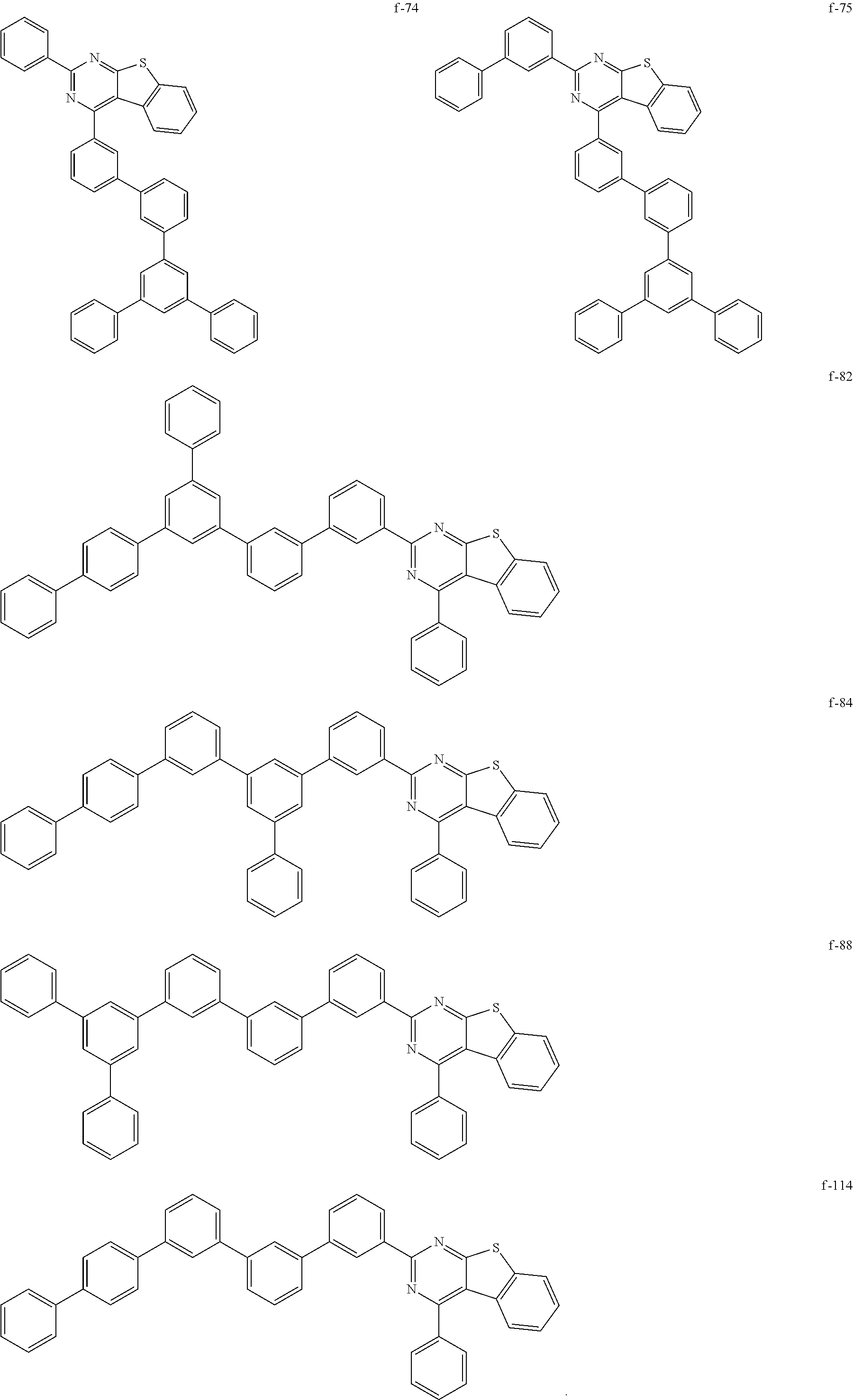

13. An organic light-emitting device comprising: a first electrode; a second electrode; and an organic layer disposed between the first electrode and the second electrode and comprising the condensed cyclic compound of claim 1.

14. The organic light-emitting device of claim 13, wherein the condensed cyclic compound of Formula 1 is included as a host in an emission layer of the organic layer or in an electron transport auxiliary layer.

15. The organic light-emitting device of claim 14, wherein the host of the emission layer further comprises at least one of a first compound represented by Formula 41 and a second compound represented by Formula 61: 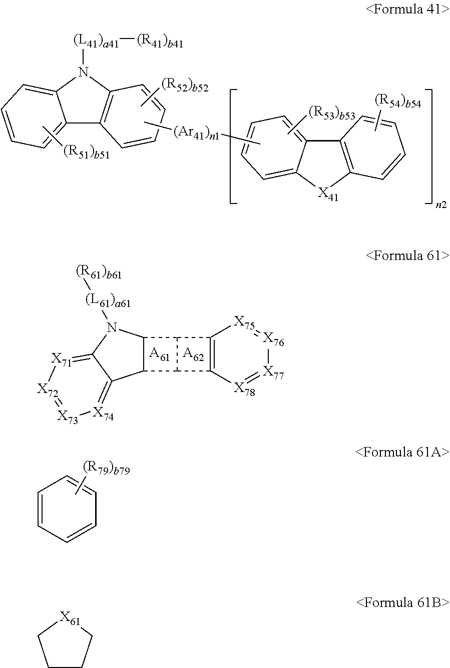
wherein, in Formulae 41 and 61,
X₄₁ is N-[(L₄₂)ₐ₄₂-(R₄₂)_{b42}], S, O, S(═O), S(═O)₂, a C(═O), a C(R₄₃)(R₄₄), Si(R₄₃)(R₄₄), P(R₄₃), P(═O)(R₄₃), or C═N(R₄₃);
Ring A₆₁ in Formula 61 is represented by Formula 61A;
Ring A₆₂ in Formula 61 is represented by Formula 61B;
X₆₁ is N-[((L₆₂)ₐ₆₂-(R₆₂)_{b62}], S, O, S(═O), S(═O)₂, a C(═O), a C(R₆₃)(R₆₄), Si(R₆₃)(R₆₄), P(R₆₃), P(═O)(R₆₃), or C═N(R₆₃);
X₇₁ is C(R₇₁) or N; X₇₂ is C(R₇₂) or N; X₇₃ is C(R₇₃) or N; X₇₄ is C(R₇₄) or N; X₇₅ is C(R₇₅) or N; X₇₆ is C(R₇₆) or N; X₇₇ is C(R₇₇) or N; X₇₈ is C(R₇₈) or N;
Ar₄₁, L₄₁, L₄₂, L₆₁, and L₆₂ are each independently selected from a substituted or unsubstituted C₃-C₁₀ cycloalkylene group, a substituted or unsubstituted heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;
n1 and n2 are each independently an integer selected from 0 to 3;
R₄₁ to R₄₄, R₅₁ to R₅₄, R₆₁ to R₆₄, and R₇₁ to R₇₉ are each independently selected from a hydrogen, a deuterium a fluoro group (—F), a chloro group (—Cl), a bromo group (—Br), an iodo group (—I), a hydroxyl group, a cyano group, an amino group, an amidino group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), and —B(Q₆)(Q₇);
a41, a42, a61, and a62 are each independently an integer selected from 0 to 3; and
b41, b42, b51 to b54, b61, b62, and b79 are each independently an integer selected from 1 to 3.

16. The organic light-emitting device of claim 15 wherein the emission layer comprises a first host, a second host, and a dopant,
wherein the first host, and the second host are differ from each other,
the first host comprises the at least one of the condensed cyclic compounds of Formula 1, and
the second host comprises at least one of a first compound represented by Formula 41 and a second compound represented by Formula 61.

17. The organic light-emitting device of claim 15,
L₆₁, and L₆₂ are each independently selected from a substituted or unsubstituted C₆-C₆₀ arylene group, a substituted or unsubstituted C₂-C₆₀ heteroarylene group, and a substituted or unsubstituted divalent non-aromatic condensed polycyclic group;
R₅₁ to R₅₄, R₆₁ to R₆₄, and R₇₁ to R₇₉ are each independently selected from
a hydrogen, a deuterium, —F, —Cl, —Br, —I, a hydroxyl group, a cyano group, an amino group, an amidino group, a substituted or unsubstituted C₁-C₂₀ alkyl group, a substituted or unsubstituted C₁-C₂₀ alkoxy group, a substituted or unsubstituted C₃-C₁₀ cycloalkyl group, a substituted or unsubstituted C₃-C₁₀ cycloalkenyl group, a substituted or unsubstituted C₆-C₂₀ aryl group, and a substituted or unsubstituted monovalent non-aromatic heterocondensed polycyclic group.

18. The organic light-emitting device of claim 15, wherein the first compound is represented by one of Formulae 41-1 to 41-12, and the second compound is represented by one of Formulae 61-1 to 61-6: 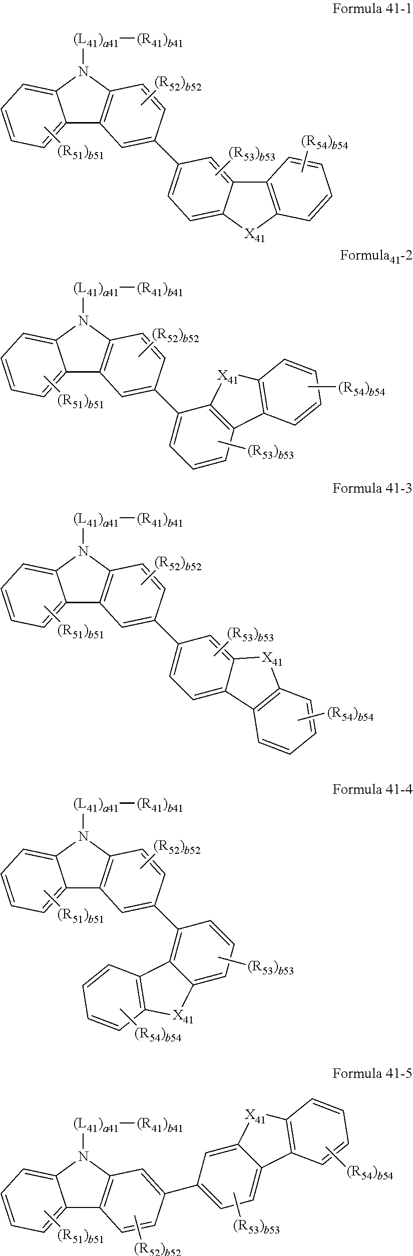 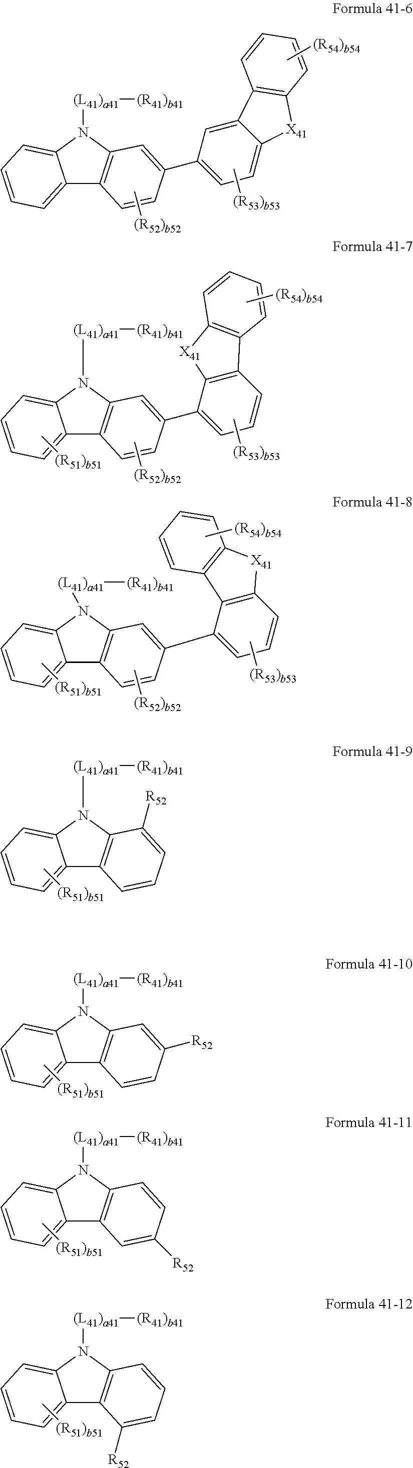 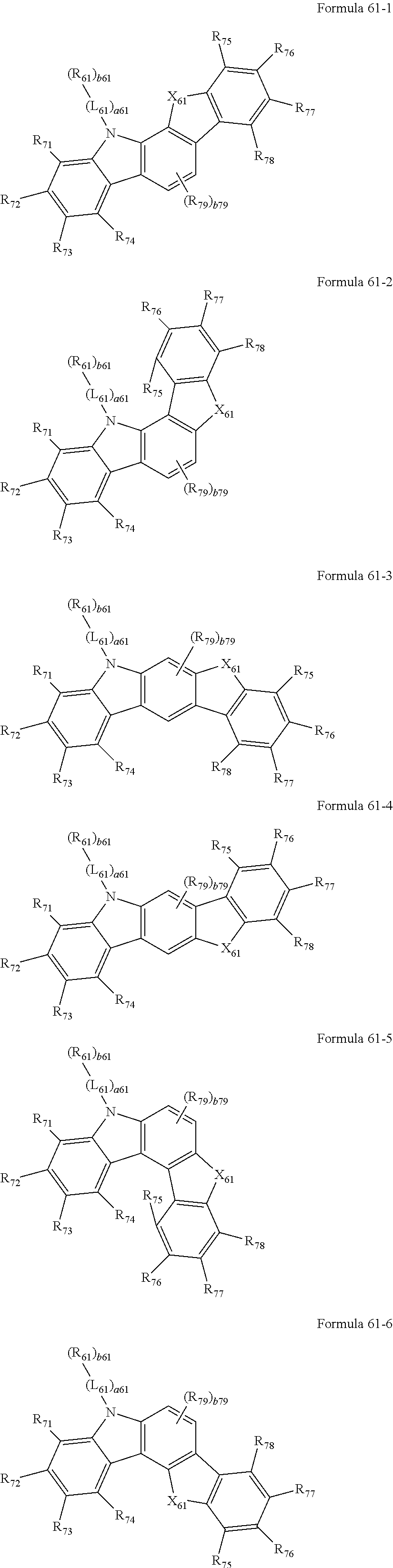
wherein, in Formulae 41-1 to 41-12 and Formulae 61-1 to 61-6, X₄₁, X₆₁, L₄₁, a41, L₆₁, a61, R₄₁, b41, b42, R₆₁, R₅₁ to R₅₄, b51 to b54, b61, b62, R₇₁ to R₇₉, and b79 are the same as those defined in claim 15.

19. The organic light-emitting device of claim 15,
wherein the condensed cyclic compound is one of Compounds listed following Group 1, and
wherein the first compound and the second compound is one of Compounds listed following Group 2:

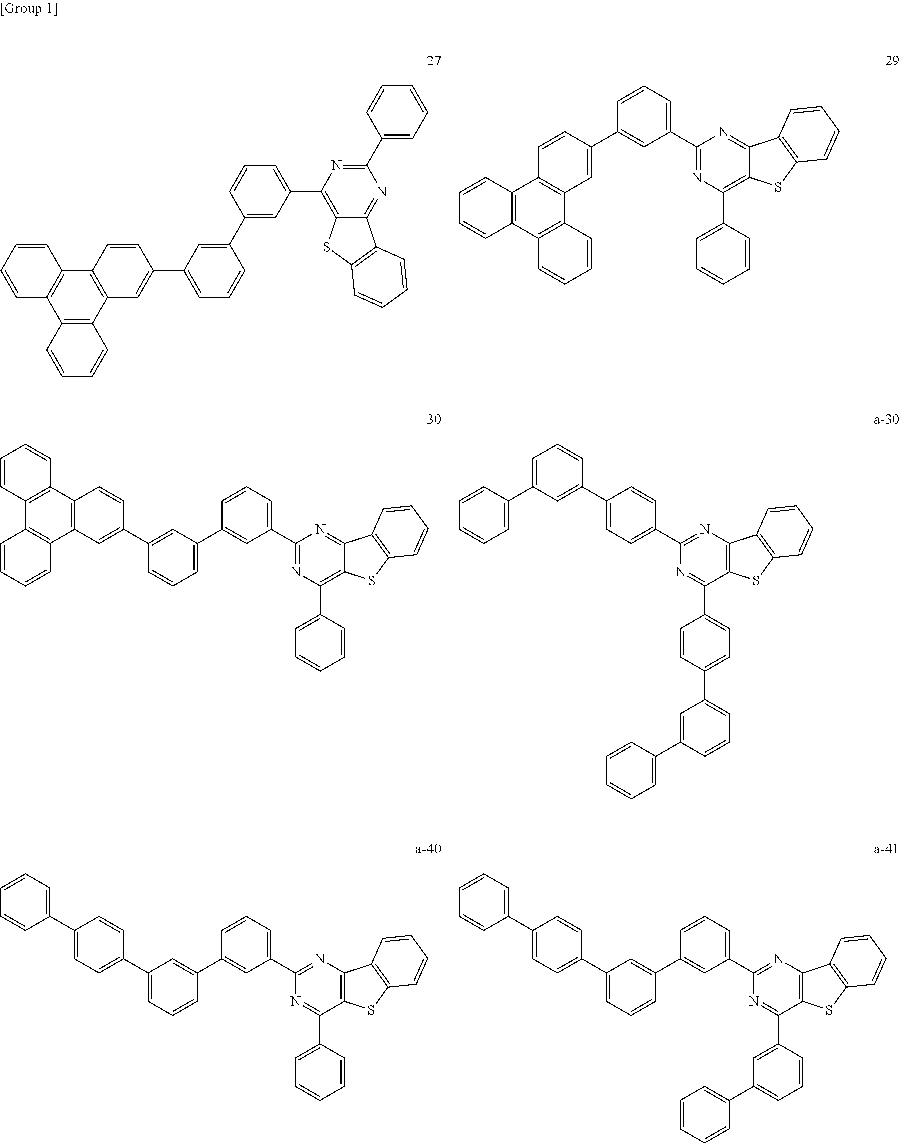

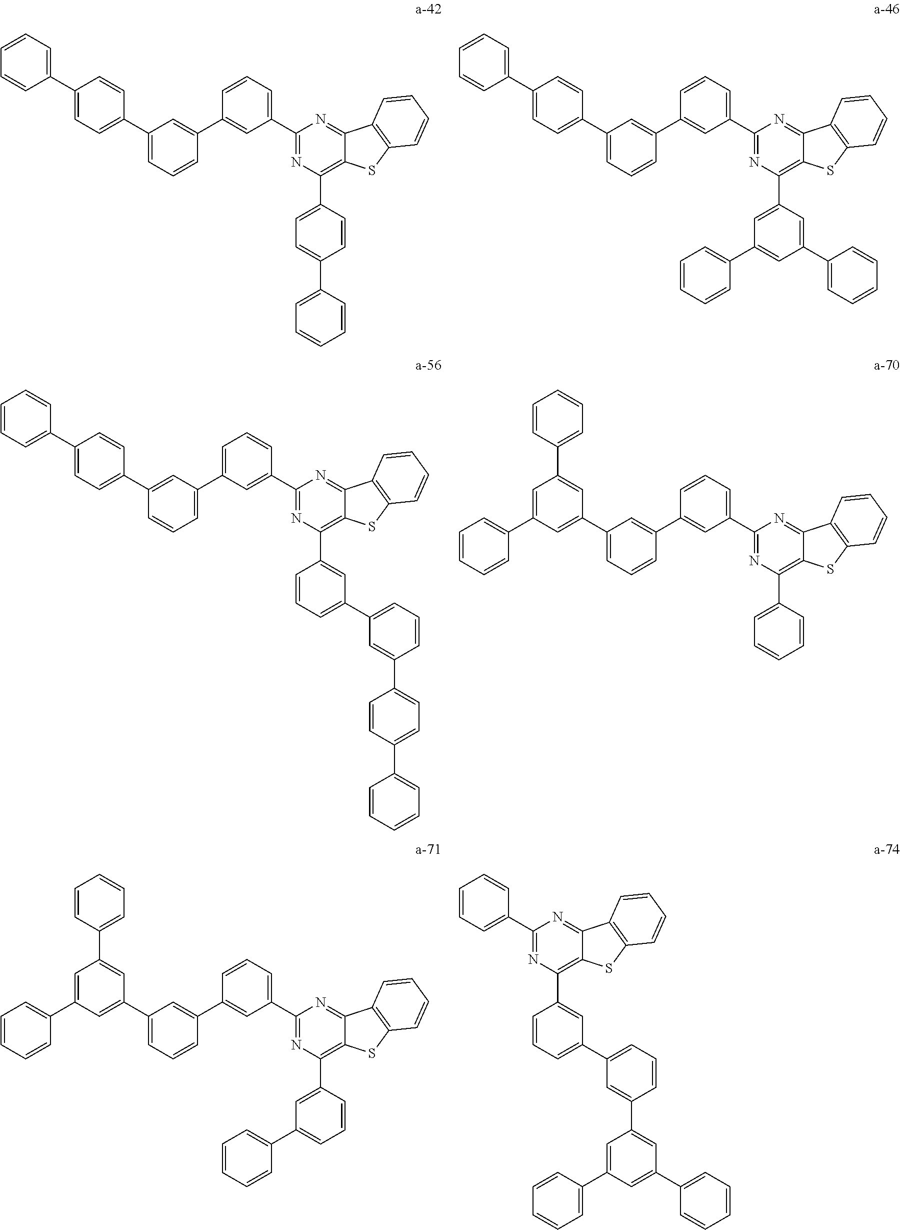

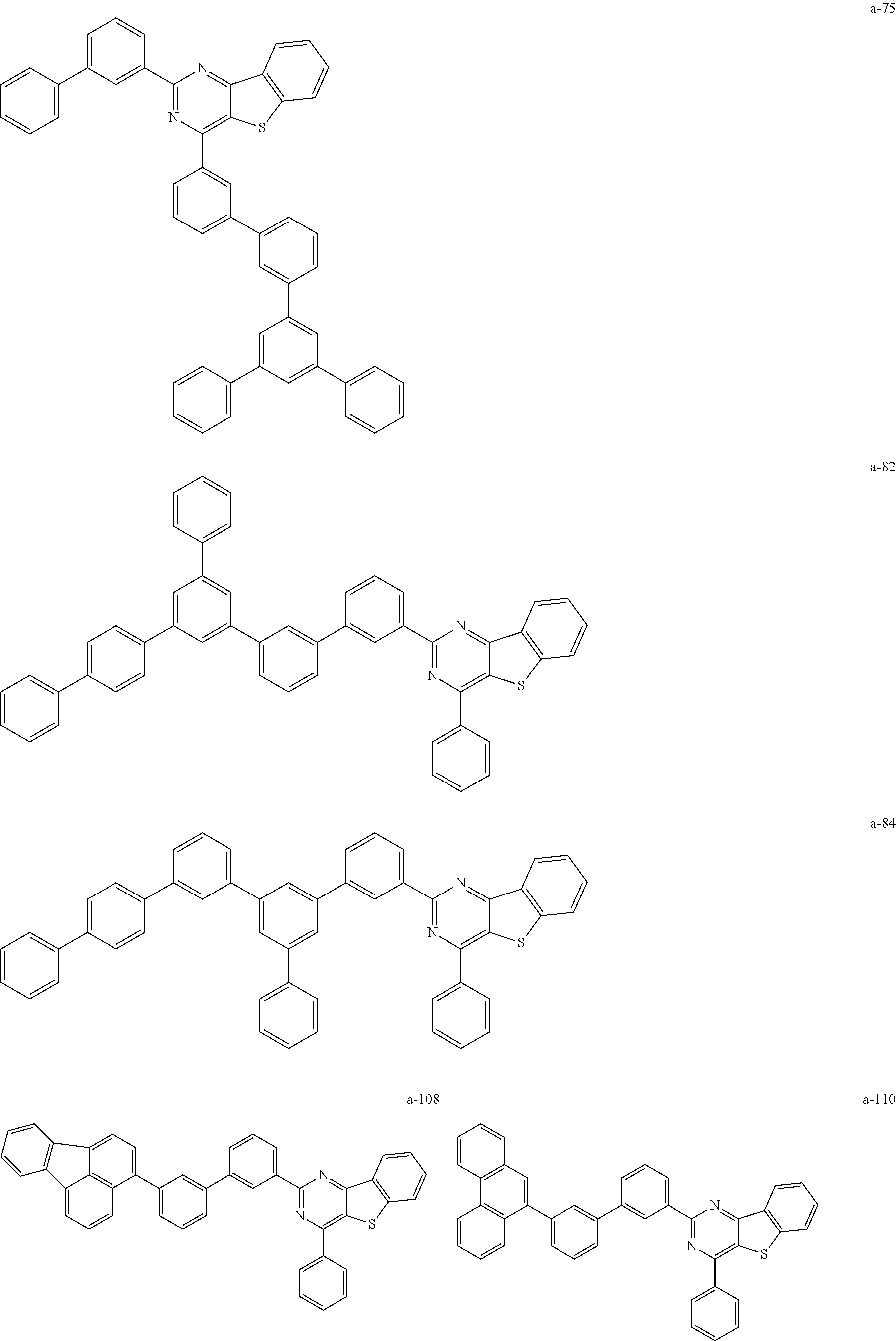

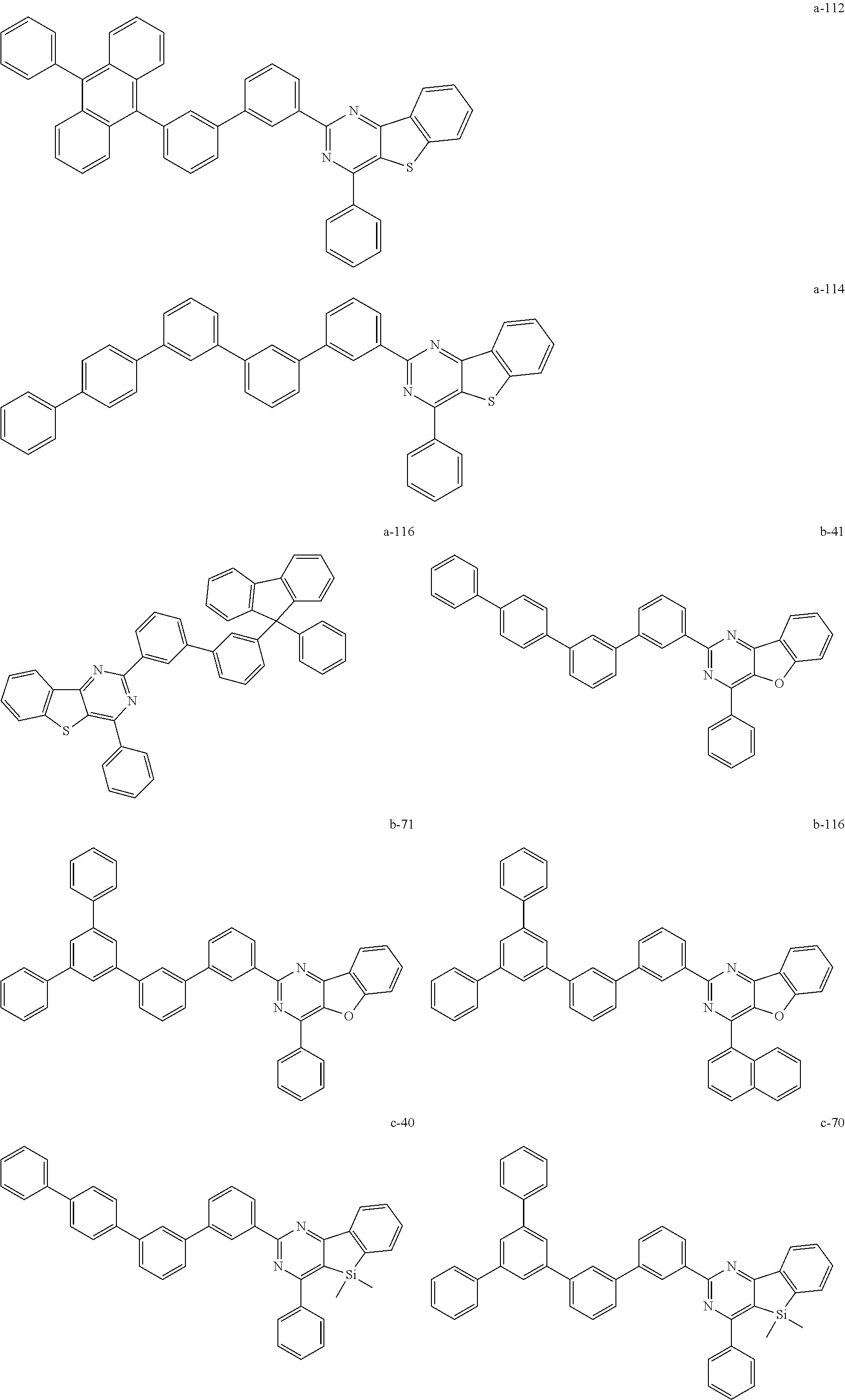

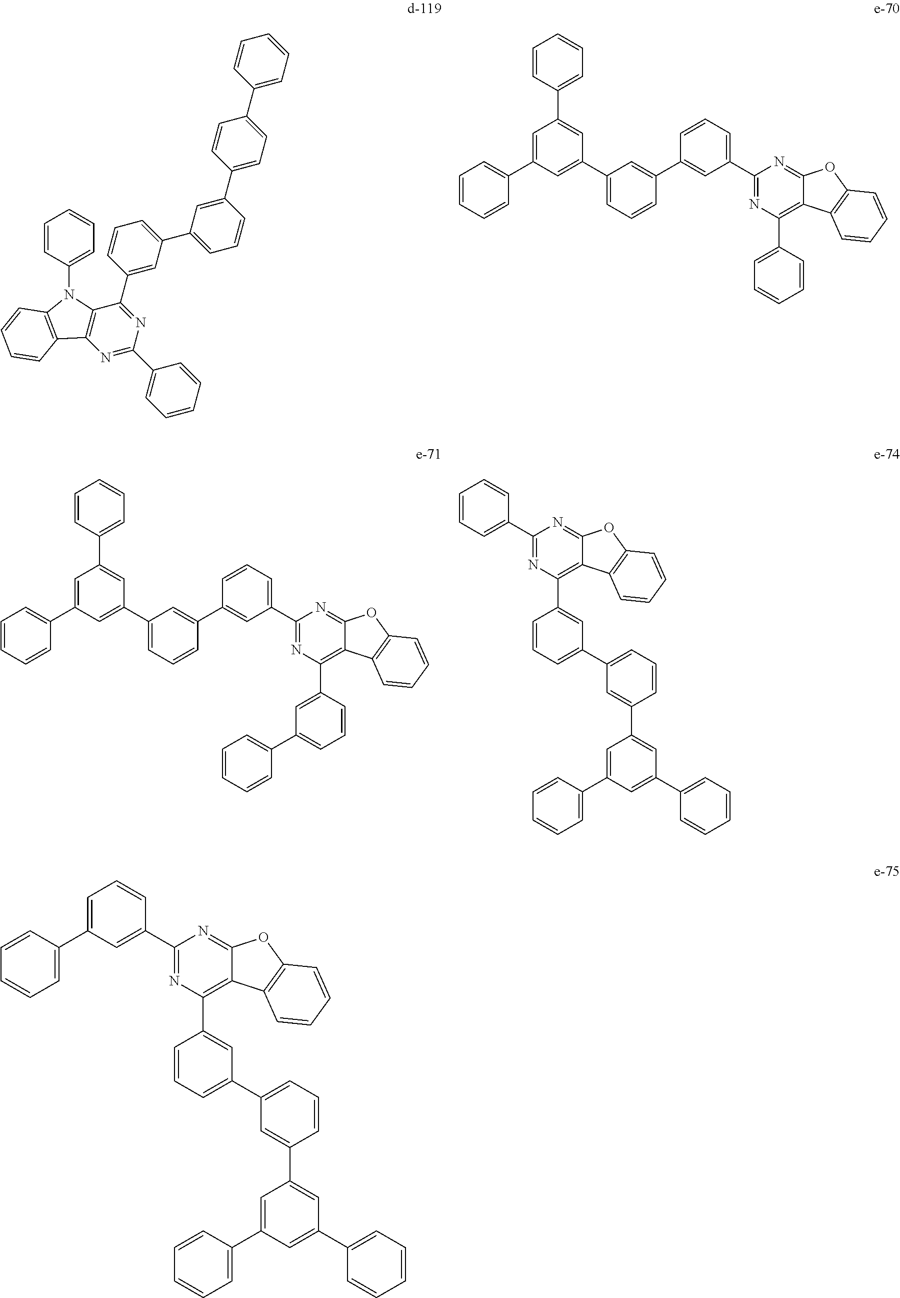 [SMILES: c1ccc(-c2ccc(-c3cccc(-c4cccc(-c5nc(-c6ccccc6)nc6c7ccccc7n(-c7ccccc7)c56)c4)c3)cc2)cc1 ; c1ccc(-c2cc(-c3ccccc3)cc(-c3cccc(-c4cccc(-c5nc(-c6ccccc6)c6c(n5)oc5ccccc56)c4)c3)c2)cc1 ; c1ccc(-c2cc(-c3ccccc3)cc(-c3cccc(-c4cccc(-c5nc(-c6cccc(-c7ccccc7)c6)c6c(n5)oc5ccccc56)c4)c3)c2)cc1 ; c1ccc(-c2cc(-c3ccccc3)cc(-c3cccc(-c4cccc(-c5nc(-c6ccccc6)nc6oc7ccccc7c56)c4)c3)c2)cc1 ; c1ccc(-c2cc(-c3ccccc3)cc(-c3cccc(-c4cccc(-c5nc(-c6cccc(-c7ccccc7)c6)nc6oc7ccccc7c56)c4)c3)c2)cc1]

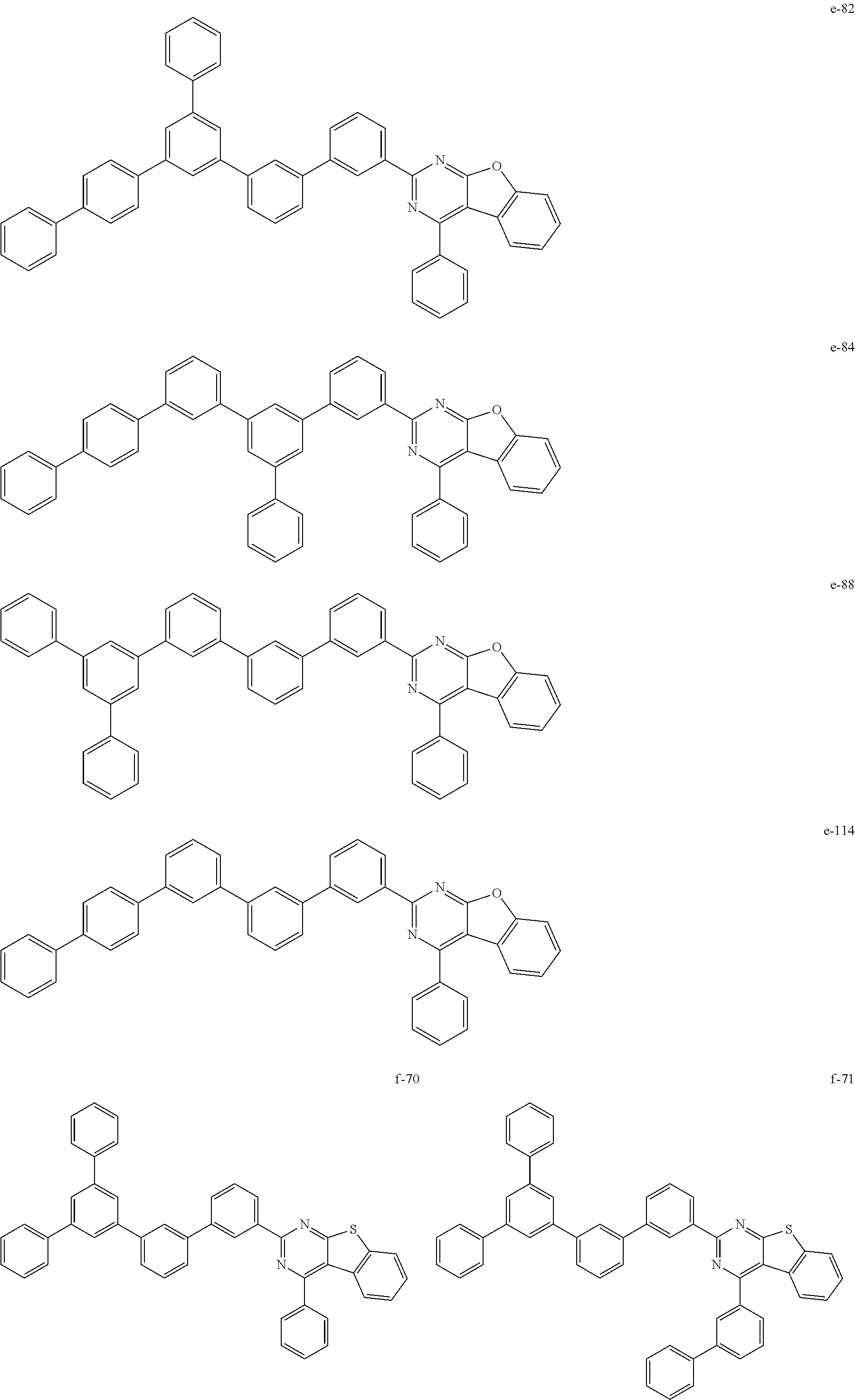

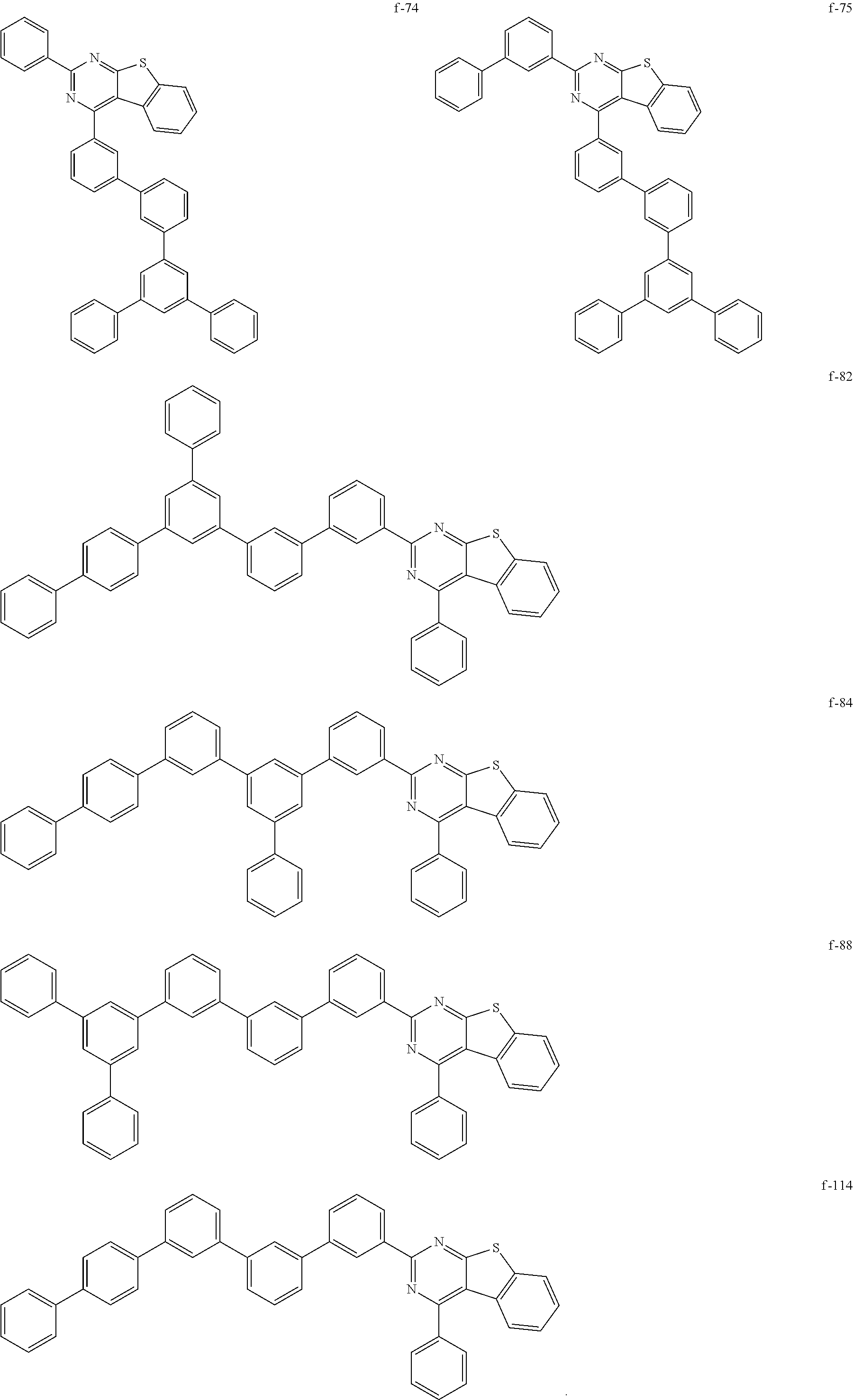

20. The organic light-emitting device of claim 14, wherein
the condensed cyclic compound is included in an electron transport auxiliary layer of the organic layer, and
the organic light-emitting device further includes a compound represented by the following Formula 2: 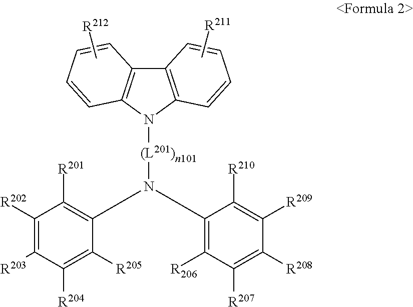
wherein, in Formula 2,
L²⁰¹ is a substituted or unsubstituted C6 to C30 arylene group, or a substituted or unsubstituted C2 to C30 heteroarylene group,
n101 is an integer of 1 to 5,
R²⁰¹ to R²¹² are each independently hydrogen, a deuterium, a substituted or unsubstituted C1 to C20 alkyl group, a substituted or unsubstituted C6 to C50 aryl group, a substituted or unsubstituted C2 to C50 heteroaryl group or a combination thereof, and
R²⁰¹ to R²¹² are each independently present or are fused to each other to form a ring.
